(19) United States
(12) Patent Application Publication
(10) Pub. No.: US 2023/0000842 A1
(43) Pub. Date: Jan. 5, 2023

(54) METHODS OF TREATING CARTILAGE DISORDERS THROUGH INHIBITION OF CLK AND DYRK

(71) Applicant: SAMUMED, LLC, San Diego, CA (US)

(72) Inventors: Vishal Deshmukh, San Diego, CA (US); Alyssa-Lauren O'Green, San Diego, CA (US); Carine Bossard, San Diego, CA (US); Charlene F. Barroga, San Diego, CA (US); John Hood, Del Mar, CA (US)

(21) Appl. No.: 17/423,761

(22) PCT Filed: Jan. 17, 2020
(86) PCT No.: PCT/US2020/013995

§ 371 Date: Jul. 16, 2021

Related U.S. Application Data
(60) Provisional application No. 62/793,855, filed on Jan. 17, 2019.

(51) Int. Cl. A61K 31/438; A61K 31/444; A61K 31/4439; A61K 31/496; A61K 31/497; A61K 31/501; A61K 31/55; A61K 31/541; A61K 31/5386; A61K 31/416; A61K 31/427; A61K 31/4985; A61K 31/437; G01N 33/68

(52) U.S. Cl. CPC A61K 31/438; A61K 31/444; A61K 31/4439; A61K 31/496; A61K 31/497; A61K 31/501; A61K 31/55; A61K 31/541; A61K 31/5386; A61K 31/416; A61K 31/427; A61K 31/4985; A61K 31/437; G01N 33/6893; G01N 2800/105

(57) ABSTRACT

Provided herein are methods of treating cartilage disorders in a subject using a dual CLK/DYRK inhibitor, or a pharmaceutically acceptable salt of solvate thereof, or a combination of a CLK inhibitor, or a pharmaceutically acceptable salt of solvate thereof, and DYRK inhibitor or, pharmaceutically acceptable salt or solvate thereof.

### RELATED APPLICATION

This application claims the benefit of U.S. Provisional Application No. 62/793,855, filed Jan. 17, 2019, which is incorporated herein by reference in its entirety.

### BACKGROUND

### Technical Field

This present disclosure relates to the fields of molecular biology, and more specifically, to methods of treating cartilage disorders using a dual CDC-like kinase (CLK)/Dual specificity tyrosine-phosphorylation-regulated kinase (DYRK) inhibitor or a combination of a CLK inhibitor and a DYRK inhibitor.

### Background

Chondrogenesis is the process that results in the formation of the cartilage intermediate, or anlagen, and leads to endochondral ossification during skeletal development. Chondrogenesis is the earliest phase of skeletal development, involving mesenchymal cell recruitment and migration, condensation of progenitors, and chondrocyte differentiation, and maturation and resulting in the formation of cartilage and bone during endochondral ossification. This process is controlled exquisitely by cellular interactions with the surrounding matrix, growth and differentiation factors, and other environmental factors that initiate or suppress cellular signaling pathways and transcription of specific genes in a temporal-spatial manner [Annual Review of Cell and Developmental Biology (2000), 16, 191-220].

Cartilage is a tough, flexible and elastic connective tissue, which has numerous functions. It is mainly composed of an abundant collagen and proteoglycan-rich extracellular matrix (ECM) in which the primary cell type of cartilage, the chondrocyte, resides. This composition gives rise to a highly hydrated tissue, which allows effective completion of its primary functions; to disperse forces on the joints during movement and to act as a template for bone formation and longitudinal bone growth [Cell Biochemistry and Function (2012), 30(8), 633-642]. More specifically, articular cartilage functions to reduce friction and to withstand the mechanical stress placed upon the ends of the long bones during joint movement. For this reason, articular cartilage is structurally adapted to fit this need. Like in the growth plate, articular cartilage is organized in a strict hierarchy, the organization, and thus, the mechanical efficiency of which increases with maturity. Articular cartilage is hypocellular, avascular, aneural and alymphatic. Chondrocytes constitute less than 5% of articular cartilage with their vast ECM comprising the rest and as such, their viability is critical. The homeostatic equilibrium of ECM synthesis and degradation is also crucial in maintaining healthy and fully functioning articular cartilage.

Wnt signaling is an evolutionary conserved pathway which plays an important role in embryonic development, cell viability, and regeneration (Cell (2012), 149(6), 1192-1205; Cell (2006), 127(3), 469-80). Signaling is activated upon Wnt ligand binding to a Frizzled family cell receptor and is transmitted via canonical (β-catenin dependent) or non-canonical (β-catenin-independent) pathways (Cell (2006), 127(3), 469-80). Activation of canonical Wnt signaling releases β-catenin from the protein complex of GSK3-β, AXIN, and adenomatous polyposis coli (APC), and promotes the proteosomal degradation of the freed β-catenin (The EMBO Journal (2012), 31(12), 2670-84). Upon subsequent translocation into the nucleus, β-catenin interacts with TCF/LEF transcription factors to activate expression of target genes important not only in cell fate, but in cell proliferation and survival (Nature Reviews Genetics (2004), 5, 691-701). The Wnt signaling pathway plays a crucial role in the development and homeostasis of a variety of adult tissues and, as such, is emerging as an important therapeutic target for numerous diseases. Factors involved in the Wnt pathway are expressed throughout limb development and chondrogenesis and have been shown to be critical in joint homeostasis and endochondral ossification.

Several Wnt genes, including Wnt4, Wnt14, and Wnt16, were expressed in overlapping and complementary patterns in the developing synovial joints, where β-catenin protein levels and transcription activity were up-regulated. Removal of β-catenin early in mesenchymal progenitor cells promoted chondrocyte differentiation and blocked the activity of Wnt14 in joint formation. Ectopic expression of an activated form of β-catenin or Wnt14 in early differentiating chondrocytes induced ectopic joint formation both morphologically and molecularly. In contrast, genetic removal of β-catenin in chondrocytes led to joint fusion. These results demonstrate that the Wnt/β-catenin signaling pathway is necessary and sufficient to induce early steps of synovial joint formation. Wnt4, Wnt14, and Wnt16 may play redundant roles in synovial joint induction by signaling through the β-catenin-mediated canonical Wnt pathway [Genes & Development (2004), 18(19), 2404-2417. Wnt signaling appears critical for not only the formation of the joint but also its maintenance as indicated by the numerous transgenic mouse models which invariably display postnatal phenotypes. Any dysregulation in the integrity of the articular cartilage can lead to its degradation, as is commonly seen in osteoarthritis [Bone (2009), 44(4), 522-527].

The repair of joint surface defects remains a clinical challenge, as articular cartilage has a limited healing response. Despite this, articular cartilage does have the capacity to grow and remodel extensively during pre- and post-natal development. As such, the elucidation of developmental mechanisms, particularly those in post-natal animals, may shed valuable light on processes that could be harnessed to develop novel approaches for articular cartilage tissue engineering and/or regeneration to treat injuries or degeneration in adult joints. In addition, osteoarthritis affected chondrocytes are frequently reported to upregulate genes normally detected during embryonic limb formation [The Journal of rheumatology (2005), 32(5), 876-886].

### SUMMARY

The present disclosure is based on the discovery that dual CLK/DYRK inhibitors can decrease the level of Wnt/β-catenin signaling activity in a mammalian cell and can modulate the process of chondrogenesis in a mammalian cell. In view of these discoveries, provided herein are methods of treating cartilage disorders in a subject, methods of selecting a treatment for a subject, methods of selecting a subject for treatment, and methods of selecting a subject for participation in a clinical trial, that can each include identifying a subject having a cartilage disorder (e.g., any of the types of cartilage disorders described herein) that has an elevated level of Wnt pathway activity as compared to a reference level.

The present disclosure provides methods of treating a disease in a subject in need thereof, the method comprising administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. In some embodiments, the disease is selected from axial spondyloarthritis, costochondritis, degenerative disc disease, degenerative spondylolisthesis, elbow dysplasia, gout, juvenile idiopathic arthritis, osteoarthritis, osteochondritis dissecans, Panner disease, reactive arthritis, relapsing polychondritis, rheumatoid arthritis, sacroiliac joint dysfunction, septic arthritis, Still's disease, Tietze syndrome, psoriasis, reactive arthritis, Ehlers-Danlos syndrome, haemochromatosis, hepatitis, Lyme disease, Sjogren's disease, Hashimoto's thyroiditis, Celiac disease, non-celiac gluten sensitivity, inflammatory bowel disease, Henoch-Schonlein purpura, hyperimmunoglobulinemia D with recurrent fever, sarcoidosis, Whipple's disease, TNF receptor associated periodic syndrome, granulomatosis with polyangiitis, familial Mediterranean fever, and systemic lupus erythematosus.

Also provided herein are methods of treating osteoarthritis in a subject in need thereof that include administering to the subject a first compound, wherein the first compound is a CLK2 and/or CLK3 inhibitor or a pharmaceutically acceptable salt or solvate thereof and a second compound, wherein the second compound is a DYRKA1 inhibitor or a pharmaceutically acceptable salt or solvate thereof.

Also provided herein are methods of inducing chondrogenesis in a subject in need thereof, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of inducing chondrocyte differentiation in a subject in need thereof, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of increasing chondrocyte function in a subject in need thereof, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of preventing cartilage breakdown in a subject in need thereof, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. For example, methods of decreasing and/or inhibiting cartilage breakdown in a subject in need thereof, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of preventing chondrocytic catabolic effects in a subject in need thereof, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. For example, methods of decreasing and/or inhibiting chondrocytic catabolic effects in a subject in need thereof, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject in need thereof, the method comprising: (a) identifying a subject, wherein the subject is identified by localized joint pain associated with at least one of inflammation and effusion; and (b) administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject in need thereof, the method comprising: (a) identifying a subject, wherein the subject is identified by at least one of NRS pain scale, WOMAC, and Kellgren-Lawrence; and (b) administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof, to a subject identified as having an elevated level of a biomarker associated with inflammation.

Also provided herein are methods of selecting a subject for treatment, the method comprising selecting a subject identified as having an elevated level of a biomarker associated with inflammation for treatment with a therapeutically effective amount of a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject, the method comprising administering a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof, to a subject identified as having an elevated level of a biomarker associated with inflammation.

Also provided herein are methods of selecting a subject for treatment, the method comprising: (a) performing a diagnostic test on the subject to confirm osteoarthritis; and (b) selecting the subject for treatment, wherein the treatment includes administration of a single compound, wherein the single compound is a dual DYRK1A CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

Also provided herein are methods of selecting a subject for treatment, the method comprising selecting a subject identified as having an elevated level of a biomarker associated with inflammation for treatment with a therapeutically effective amount of a single compound, wherein the single compound is a dual DYRK1A/CLK2 and/or CLK3 inhibitor, which inhibits DYRK1A at an IC₅₀ value of less than 100 nM and CLK2 and/or CLK3 at an IC₅₀ value of less than 100 nM, or a pharmaceutically acceptable salt or solvate thereof.

Also provided herein are methods of treating osteoarthritis in a subject in need thereof, the method comprising: (a) detecting an elevated level of Wnt pathway activity in a sample from the subject, as compared to a reference level; and (b) administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of modifying the progression of osteoarthritis in a subject in need thereof, the method comprising: (a) detecting an elevated level of Wnt pathway activity in a sample from the subject, as compared to a reference level; and (b) administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject in need thereof, the method comprising: (a) identifying a subject having an elevated level of Wnt pathway activity in a sample from the subject, as compared to a reference level; and (b) administering to the subject a therapeutically effective amount of a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject, the method comprising administering a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof, to the subject, wherein the subject is identified as having an elevated level of Wnt pathway activity in a sample from the subject as compared to a reference level.

Also provided herein are methods of selecting a subject for treatment, the method comprising: (a) detecting an elevated level of Wnt pathway activity in a sample from the subject, as compared to a reference level; and (b) selecting the subject for treatment, wherein the treatment includes administration of a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of selecting a subject for treatment, the method comprising selecting a subject identified as having an elevated level of Wnt pathway activity in a sample from a subject as compared to a reference level, for treatment with a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject in need thereof, the method comprising: (a) detecting an elevated level of a biomarker associated with inflammation in a sample of the subject; and (b) administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject in need thereof, the method comprising: (a) identifying a subject, wherein the subject is identified when a sample of the subject has an elevated level of a biomarker associated with inflammation; and (b) administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of selecting a subject for treatment, the method comprising: (a) detecting an elevated level of a biomarker associated with inflammation in a sample of the subject; and (b) selecting the subject for treatment, wherein the treatment includes administration of a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof.

Also provided herein are methods of treating osteoarthritis in a subject in need thereof, the method comprising: (a) detecting an elevated level of Wnt pathway activity in a sample from the subject, as compared to a reference level; and (b) administering to the subject a therapeutically effective amount of a single compound, wherein the single compound is a dual DYRK1A/CLK2 and/or CLK3 inhibitor, which inhibits DYRK1A at an IC₅₀ value of less than 100 nM and CLK2 and/or CLK3 at an IC₅₀ value of less than 100 nM, or a pharmaceutically acceptable salt or solvate thereof.

Also provided herein are methods of treating osteoarthritis in a subject in need thereof, the method comprising: (a) detecting an elevated level of Wnt pathway activity in a sample from the subject, as compared to a reference level; and (b) administering to the subject a therapeutically effective amount of a single compound, wherein the single compound is a dual DYRK1A/CLK2 and/or CLK3 inhibitor, which inhibits DYRK1A at an IC₅₀ value of less than 100 nM and CLK2 and/or CLK3 at an IC₅₀ value of less than 100 nM, or a pharmaceutically acceptable salt or solvate thereof.

Also provided herein are methods of treating osteoarthritis in a subject in need thereof, the method comprising: (a) identifying a subject, wherein the subject has an elevated level of Wnt pathway activity in a sample from the subject, as compared to a reference level; and (b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

Also provided herein are methods of modifying the progression of osteoarthritis in a subject in need thereof, the method comprising: (a) identifying a subject, wherein the subject has an elevated level of Wnt pathway activity in a sample from the subject, as compared to a reference level; and (b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

Also provided herein are methods of treating osteoarthritis in a subject, the method comprising administering a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof, to a subject identified as having an elevated level of Wnt pathway activity in a sample from the subject, as compared to a reference level.

Also provided herein are methods of selecting a subject for treatment, the method comprising: (a) detecting an elevated level of Wnt pathway activity in a sample from the subject, as compared to a reference level; and (b) selecting the subject for treatment, wherein the treatment includes administration of a single compound, wherein the single compound inhibits CLK2 and/or CLK3 at an IC₅₀ value of less than 100 nM and DYRK1A at an IC₅₀ value of less than 100 nM, or a pharmaceutically acceptable salt or solvate thereof.

Also provided herein are methods of selecting a subject for treatment, the method comprising selecting a subject identified as having an elevated level of Wnt pathway activity in a sample from the subject as compared to a reference level, and treating the subject with a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

Also provided herein are methods of inducing chondrogenesis in a subject in need thereof, the method comprising: (a) detecting an elevated level of Wnt pathway activity in a sample from the subject, as compared to a reference level; and (b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof, wherein the subject exhibits chondrogenesis.

Also provided herein are methods of treating osteoarthritis in a subject in need thereof, the method comprising: (a) detecting an elevated level of a biomarker associated with inflammation in a sample of the subject; and (b) administering to the subject a therapeutically effective amount of a single compound, wherein the single compound is a dual DYRK1A/CLK2 and/or CLK3 inhibitor, which inhibits CLK2 at an IC₅₀ value of less than 100 nM and DYRK1A at an IC₅₀ value of less than 100 nM, or a pharmaceutically acceptable salt or solvate thereof.

Also provided herein are methods of treating osteoarthritis in a subject in need thereof, the method comprising: (a) identifying a subject, wherein the subject is identified when a sample of the subject has an elevated level of a biomarker associated with inflammation; and (b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

Also provided herein are methods of selecting a subject for treatment, the method comprising: (a) detecting an elevated level of a biomarker associated with inflammation in a sample of the subject; and (b) selecting the subject for treatment, wherein the treatment includes administration of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

In some embodiments of any of the methods described herein, the dual CLK/DYRK inhibitor, CLK inhibitor, or DYRK inhibitor is a compound of any one of Formulas (I)-(VIII) or a pharmaceutically acceptable salt or solvate thereof.

In some embodiments of any of the methods described herein, the dual CLK/DYRK inhibitor, CLK inhibitor, or DYRK inhibitor is a compound of Formula (I)

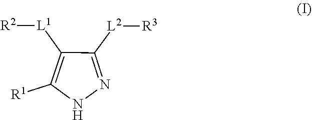

or a pharmaceutically acceptable salt or solvate thereof, wherein:

R¹ is selected from the group consisting of H, halide, and unsubstituted —(C₁₋₃ alkyl);

R² is selected from the group consisting of unsubstituted —(C₁₋₉ alkyl), unsubstituted —(C₂₋₉ alkenyl), unsubstituted —(C₁₋₉ haloalkyl), —(C₁₋₂ alkylene)ₚ(C₃₋₆ carbocyclyl) optionally substituted with 1-12 R⁴, -monocyclic heterocyclyl optionally substituted with 1-10 R⁵, -phenyl optionally substituted with 1-5 R⁶, -heteroaryl optionally substituted with 1-4 R⁷, —CO₂R⁸, —OR⁹, and —(C═O)R¹⁰; wherein —(C₁₋₄ alkylene) is optionally substituted with one or more substituents as defined anywhere herein;

R³ is selected from the group consisting of -heterocyclyl optionally substituted with 1-10 R¹¹, —(C₁₋₄ alkylene)ₚphenyl optionally substituted with 1-5 R¹², -heteroaryl optionally substituted with 1-4 R¹³, and —(C₁₋₄ alkylene)OR¹⁴; wherein each —(C₁₋₄ alkylene) is, independently, optionally substituted with one or more substituents as defined anywhere herein;

each R⁴ is halide;

each R⁵ is independently selected from the group consisting of halide and unsubstituted —(C₁₋₉ alkyl);

each R⁶ is independently selected from the group consisting of unsubstituted —(C₁₋₉ alkyl), unsubstituted —(C₁₋₉ haloalkyl), —OR¹⁵, and —(C₁₋₄ alkylene)ₚN(R¹⁶)₂; wherein —(C₁₋₄ alkylene) is optionally substituted with one or more substituents as defined anywhere herein;

each R⁷ is independently selected from the group consisting of halide, unsubstituted —(C₁₋₉ alkyl), unsubstituted —(C₁₋₉ haloalkyl), —OR¹⁵, —CO₂R¹⁷, —NR¹⁸(C═O)R¹⁹, —(C₁₋₄ alkylene)ₚheterocyclyl optionally substituted with 1-10 R²⁰, and —(C₁₋₄ alkylene)ₚN(R¹⁶)₂; wherein each —(C₁₋₄ alkylene) is, independently, optionally substituted with one or more substituents as defined anywhere herein;

R⁸ is unsubstituted —(C₁₋₉ alkyl);

R⁹ is unsubstituted —(C₁₋₉ alkyl);

R¹⁰ is -aryl optionally substituted with 1-5 R²¹;

each R¹¹ is independently selected from the group consisting of halide and unsubstituted —(C₁₋₉ alkyl);

each R¹² is independently selected from the group consisting of —(C₁₋₄ alkylene)ₚheterocyclyl optionally substituted with 1-10 R²⁰, -aryl optionally substituted with 1-5 R²², —(C₁₋₄ alkylene)N(R¹⁶)₂, and —OR²³; wherein each —(C₁₋₄ alkylene) is, independently, optionally substituted with one or more substituents as defined anywhere herein;

each R¹³ is independently selected from the group consisting of halide, unsubstituted —(C₁₋₉ alkyl), unsubstituted —(C₁₋₉ haloalkyl), —(C₁₋₄ alkylene)ₚN(R¹⁶)₂, —OR²³, —(C₁₋₄ alkylene)ₚheterocyclyl optionally substituted with 1-10 R²⁰, -aryl optionally substituted with 1-5 R²², and -heteroaryl optionally substituted with 1-4 R²⁴; wherein —(C₁₋₄ alkylene) is optionally substituted with one or more substituents as defined anywhere herein;

R¹⁴ is selected from the group consisting of unsubstituted —(C₁₋₄ alkyl) and -aryl optionally substituted with 1-5 R²²;

each R¹⁵ is independently selected from the group consisting of unsubstituted —(C₁₋₉ alkyl) and -heterocyclyl optionally substituted with 1-10 R²⁰;

each R¹⁶ is independently selected from the group consisting of H and unsubstituted —(C₁-9 alkyl);

each R¹⁷ is unsubstituted —(C₁₋₉ alkyl);

each R¹⁸ is independently selected from the group consisting of H and unsubstituted —(C₁₋₉ alkyl);

each R¹⁸ is unsubstituted —(C₁₋₉ alkyl);

each R²⁰ is independently selected from the group consisting of halide and unsubstituted —(C₁₋₉ alkyl);

each R²¹ is independently selected from the group consisting of halide and unsubstituted —(C₁₋₉ alkyl);

each R²² is independently selected from the group consisting of halide and unsubstituted —(C₁₋₉ alkyl);

each R²³ is independently selected from the group consisting of unsubstituted —(C₁₋₉ alkyl), —(C₁₋₄ alkylene)OR²⁵, and —(C₁₋₄ alkylene)ₚheterocyclyl optionally substituted with 1-10 R²⁰; wherein each —(C₁₋₄ alkylene) is, independently, optionally substituted with one or more substituents as defined anywhere herein;

each R²⁴ is independently selected from the group consisting of halide and unsubstituted —(C₁₋₉ alkyl);

each R²⁵ is independently selected from the group consisting of H and unsubstituted —(C₁₋₉ alkyl);

L¹ is selected from the group consisting of a bond, —CH═CH—, —CH≡CH—, —(CH₂)ₚNR¹⁸(C═O)—, —(C═O)NR¹⁸(CH₂)ₚ—, —NR¹⁸(C═O)NR¹⁸—, —NH(CH₂)ₚ—, and —(CH₂)ₚNH—;

L² is selected from the group consisting of a bond, —(C═O)NR¹⁸-, —NR¹⁸(C═O)—, —NHCH₂—, and —CH₂NH—; and

each p is independently an integer of 0 or 1.

In some embodiments of any of the methods described herein, the dual CLK/DYRK inhibitor, CLK inhibitor, or DYRK inhibitor is a compound of Formula (II):

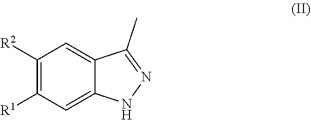

or a pharmaceutically acceptable salt or solvate thereof, wherein:

R¹ is selected from the group consisting of H and halide;

R² is a 6-membered -heteroaryl optionally substituted with 1-4 R³;

each R³ is selected from the group consisting of —OR⁴, —NHR⁵, and —(C₁₋₄ alkylene)ₚheterocyclyl optionally substituted with 1-10 R⁶; wherein —(C₁₋₄ alkylene) is optionally substituted with one or more substituents as defined anywhere herein;

each R⁴ is independently selected from the group consisting of -heterocyclyl optionally substituted with 1-10 R⁷ and —CH₂CH(R⁸)NH₂;

each R⁵ is independently selected from the group consisting of —(C₁₋₄ alkylene)ₚheterocyclyl optionally substituted with 1-10 R⁹ and -carbocyclyl optionally substituted with 1-12 R¹⁰; wherein —(C₁₋₄ alkylene) is optionally substituted with one or more substituents as defined anywhere herein;

each R⁶ is independently selected from the group consisting of halide, —NH₂, —OH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁷ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁸ is independently selected from the group consisting of —(C₁₋₄ alkylene)aryl optionally substituted with 1-5 R¹¹ and —(C₁₋₄ alkylene)heteroaryl optionally substituted with 1-4 R¹²; wherein each —(C₁₋₄ alkylene) is, independently, optionally substituted with one or more substituents as defined anywhere herein;

each R⁹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹⁰ is independently selected from the group consisting of halide, —OH, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹² is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); and

each p is independently 0 or 1.

In some embodiments of any of the methods described herein, the dual CLK/DYRK inhibitor, CLK inhibitor, or DYRK inhibitor is a compound of Formula (III):

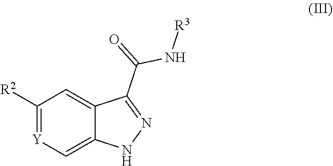

or a pharmaceutically acceptable salt or solvate thereof, wherein:

R¹ is selected from the group consisting of H, halide, and methyl;

R² is a -heteroaryl optionally substituted with 1-4 R⁴;

R³ is selected from the group consisting of H, -aryl optionally substituted with 1-5 R⁵, -heteroaryl optionally substituted with 1-4 R⁶, —C₁₋₆ alkyl optionally substituted with (i) phenyl optionally substituted with 1-5 R¹¹ or (ii) —OR¹⁵, and -carbocyclyl optionally substituted with phenyl;

each R⁴ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚN(R⁷)(R⁸), —NHC(═O)R⁹, —(C₁₋₄ alkylene)ₚOR¹⁰, unsubstituted -carbocyclyl, —(C₁₋₄ alkylene)ₚheterocyclyl optionally substituted with 1-10 R¹⁴, —(C₁₋₄ alkylene)ₚaryl optionally substituted with 1-5 R¹¹, and —(C₁₋₄ alkylene)ₚheteroaryl optionally substituted with 1-4 R¹²; wherein each —(C₁₋₄ alkylene) is, independently, optionally substituted with one or more substituents as defined anywhere herein;

each R⁵ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;

each R⁶ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;

each R⁷ is independently selected from the group consisting of H, unsubstituted —(C₁₋₆ alkyl), unsubstituted —(C₂₋₆ alkenyl), and unsubstituted —(C₂₋₆ alkynyl);

each R⁸ is independently selected from the group consisting of H, unsubstituted —(C₁₋₆ alkyl), unsubstituted —(C₂₋₆ alkenyl), unsubstituted —(C₂₋₆ alkynyl), and -heterocyclyl optionally substituted with 1-10 R²¹;

alternatively, R⁷ and R⁸ are taken together to form a -heterocyclyl ring optionally substituted with 1-10 R²¹;

each R⁹ is independently selected from the group consisting of —N(R²²)₂, -carbocyclyl optionally substituted with 1-12 R²³, -heterocyclyl optionally substituted with 1-10 R²¹, and -aryl optionally substituted with 1-5 R²⁴;

each R¹⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R²¹;

each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹² is independently selected from the group consisting of halide, —(C₁₋₄ alkylene)ₚOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;

each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹⁴ is independently selected from the group consisting of halide, —(C₁₋₄ alkylene)ₚOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;

each R¹⁵ is independently selected from the group consisting of H, unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;

alternatively, two adjacent R¹⁵ are taken together to form a -heterocyclyl ring optionally substituted with 1-10 R²¹;

each R¹⁶ is independently selected from the group consisting of unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;

each R¹⁷ is independently selected from the group consisting of H, unsubstituted —(C₁₋₆ alkyl), unsubstituted —(C₂₋₆ alkenyl), and unsubstituted —(C₂₋₆ alkynyl);

each R¹⁸ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)NMe₂, and -heterocyclyl ring optionally substituted with 1-10 R²¹; wherein —(C₁₋₄ alkylene) is optionally substituted with one or more substituents as defined anywhere herein;

each R¹⁹ is independently selected from the group consisting of unsubstituted —(C₁₋₆ alkyl), unsubstituted —(C₂₋₆ alkenyl), and unsubstituted —(C₂₋₆ alkynyl).

each R²⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —CH(CH₂OH)₂, —(C₁₋₄ alkylene)ₚheterocyclyl ring optionally substituted with 1-10 R²¹, and -aryl optionally substituted with 1-5 R²⁴; wherein —(C₁₋₄ alkylene) is optionally substituted with one or more substituents as defined anywhere herein;

each R²¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²² is independently selected from the group consisting of unsubstituted —(C₁₋₆ alkyl), unsubstituted —(C₂₋₆ alkenyl), and unsubstituted —(C₂₋₆ alkynyl);

each R²³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²⁴ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

Y is selected from the group consisting of —C(R¹)═ and —N═;

each p is independently 0 or 1.

In some embodiments of any of the methods described herein, the dual CLK/DYRK inhibitor, CLK inhibitor, or DYRK inhibitor is a compound of Formula (IV):

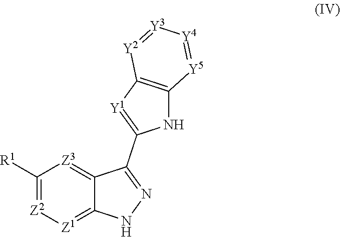

or a pharmaceutically acceptable salt or solvate thereof, wherein:

R¹ is a -heteroaryl optionally substituted with 1-2 R³;

R² is selected from the group consisting of H, halide, -aryl optionally substituted with 1-5 R⁴-heteroaryl optionally substituted with 1-4 R⁵, and -heterocyclyl ring optionally substituted with 1-10 R⁶;

each R³ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R⁷, —C(═O)N(R⁸)₂, —NHC(═O)R⁹, —(C₁₋₄ alkylene)ₚN(R¹⁰)(R¹¹), —(C₁₋₄ alkylene)ₚOR¹², and -carbocyclyl optionally substituted with 1-12 R¹³; wherein each —(C₁₋₄ alkylene) is, independently, optionally substituted with one or more substituents as defined anywhere herein;

each R⁴ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚNHSO₂R¹⁴, —NR¹⁵(C₁₋₄ alkylene)NR¹⁵R¹⁶, —(C₁₋₄ alkylene)ₚNR¹⁵R¹⁶, —OR¹⁷, and -heterocyclyl optionally substituted with 1-10 R¹⁹; wherein each —(C₁₋₄ alkylene) is, independently, optionally substituted with one or more substituents as defined anywhere herein;

each R⁵ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and —C(═O)R¹⁸;

each R⁶ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁷ is independently selected from the group consisting of halide, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁸ is independently selected from the group consisting of H, unsubstituted —(C₁₋₆ alkyl), unsubstituted —(C₂₋₆ alkenyl), unsubstituted —(C₂₋₆ alkynyl), -heterocyclyl optionally substituted with 1-10 R¹⁹, —(C₁₋₄ alkylene)ₚcarbocyclyl optionally substituted with 1-12 R²⁰; wherein —(C₁₋₄ alkylene) is optionally substituted with one or more substituents as defined anywhere herein;

each R⁹ is independently selected from the group consisting of unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²²)₂; wherein each —(C₁₋₄ alkylene) is, independently, optionally substituted with one or more substituents as defined anywhere herein;

each R¹⁰ is independently selected from the group consisting of H, unsubstituted —(C₁₋₆ alkyl), unsubstituted —(C₂₋₆ alkenyl), and unsubstituted —(C₂₋₆ alkynyl);

each R¹¹ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)ₚcarbocyclyl optionally substituted with 1-12 R²⁰; and —(C₁₋₄ alkylene)ₚaryl optionally substituted with 1-5 R²¹; wherein each —(C₁₋₄ alkylene) is, independently, optionally substituted with one or more substituents as defined anywhere herein;

each R¹² is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²²)₂; wherein each —(C₁₋₄ alkylene) is, independently, optionally substituted with one or more substituents as defined anywhere herein;

each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹⁴ is independently selected from the group consisting of unsubstituted —(C₁₋₆ alkyl), unsubstituted —(C₂₋₆ alkenyl), and unsubstituted —(C₂₋₆ alkynyl);

each R¹⁵ is independently selected from the group consisting of H, unsubstituted —(C₁₋₆ alkyl), unsubstituted —(C₂₋₆ alkenyl), and unsubstituted —(C₂₋₆ alkynyl);

each R¹⁶ is independently selected from the group consisting of H, unsubstituted —(C₁₋₆ alkyl), unsubstituted —(C₂₋₆ alkenyl), and unsubstituted —(C₂₋₆ alkynyl);

each R¹⁷ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, and, —(C₁₋₄ alkylene)ₚN(R²²)₂; wherein each —(C₁₋₄ alkylene) is, independently, optionally substituted with one or more substituents as defined anywhere herein;

each R¹⁸ is independently selected from the group consisting of unsubstituted —(C₁₋₆ alkyl), unsubstituted —(C₂₋₆ alkenyl), and unsubstituted —(C₂₋₆ alkynyl);

each R¹⁹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²⁰ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²² is independently selected from the group consisting of H, unsubstituted —(C₁₋₆ alkyl), unsubstituted —(C₂₋₆ alkenyl), and unsubstituted —(C₂₋₆ alkynyl);

each R²³ is independently selected from the group consisting of H and halide;

R²⁴ is selected from the group consisting of H, halide, and —OR¹⁷;

Y¹ is selected from the group consisting of —CH═ and —N═;

Y² is selected from the group consisting of —C(R²)═ and —N═;

with the proviso that when Y¹ is —N═ then Y² is —C(R²)═;

Y³ is selected from the group consisting of —C(R²⁴)═ and —N═;

Y⁴ and Y⁵ are independently selected from the group consisting of —C(R²³)═ and —N═;

Z¹, Z², and Z³ are independently selected from the group consisting of —C(R²³)═ and —N═;

if Y² is nitrogen then Y³, Y⁴, and Y⁵ are carbon, and R² is absent;

if Y³ is nitrogen then Y⁴ and Y⁵ are carbon;

if Y⁴ is nitrogen then Y³ and Y⁵ are carbon;

if Y⁵ is nitrogen then Y³ and Y⁴ are carbon;

if Z¹ is nitrogen then Z² and Z³ are carbon;

if Z² is nitrogen then Z¹ and Z³ are carbon;

if Z³ is nitrogen then Z¹ and Z² are carbon; and

each p is independently 0 or 1.

In some embodiments of any of the methods described herein, the dual CLK/DYRK inhibitor, CLK inhibitor, or DYRK inhibitor is a compound of Formula (V):

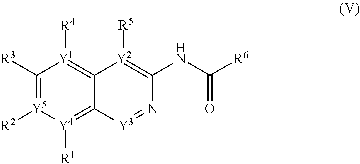

or a pharmaceutically acceptable salt or solvate thereof, wherein:

R¹, R², R⁴, and R⁵ are independently absent or selected from the group consisting of H, halide, unsubstituted —(C₁₋₃ haloalkyl), and unsubstituted —(C₁₋₃ alkyl);

R³ is selected from the group consisting of -aryl optionally substituted with 1-5 R⁷ and -heteroaryl optionally substituted with 1-4 R⁸;

R⁶ is selected from the group consisting of —(C₁₋₄ alkylene)ₚaryl optionally substituted with 1-5 R⁹, —(C₂₋₄ alkenylene)ₚaryl optionally substituted with 1-5 R⁹, —(C₁₋₄ alkylene)ₚheteroaryl optionally substituted with 1-6 R¹⁰; —(C₁₋₄ alkylene)ₚheterocyclyl optionally substituted with 1-10 R¹¹, —(C₁₋₄ alkylene)ₚcarbocyclyl optionally substituted with 1-12 R¹², —(C₁₋₄ alkylene)N(R¹³)(R¹⁴), —N(R¹⁵)(R¹⁶), —CF(C₁₋₉ alkyl)₂, —(C₁₋₄ alkylene)ₚO(C₃₋₉ alkyl), and —(C₂₋₉ alkynyl) optionally substituted with one or more halides; wherein each alkyl of —CF(C₁₋₉ alkyl)₂ is, independently, optionally substituted with one or more halides; wherein each —(C₁₋₄ alkylene) is, independently, optionally substituted with one or more substituents as defined anywhere herein; wherein —(C₁₋₄ alkenylene) is, optionally substituted with one or more substituents as defined anywhere herein;

R⁷ is selected from the group consisting of halide and —N(R¹⁷)₂;

each R⁸ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —N(R¹⁵)(R¹⁸), —(C₁₋₄ alkylene)ₚXR¹⁹, —C(═O)N(R¹⁵)₂, —(C₁₋₄ alkylene)ₚheterocyclyl optionally substituted with 1-10 R²⁰, and -carbocyclyl optionally substituted with 1-12 R²¹; wherein each —(C₁₋₄ alkylene) is, independently, optionally substituted with one or more substituents as defined anywhere herein;

alternatively, two adjacent R⁸ are taken together to form a ring which is selected from the group consisting of -heterocyclyl optionally substituted with 1-10 R²² and -carbocyclyl optionally substituted with 1-12 R²¹;

each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —XR²³, —C(═O)N(R¹⁵)₂, —(C₁₋₄ alkylene)ₚN(R²⁴)₂, —(C₁₋₄ alkylene)ₚheterocyclyl optionally substituted with 1-10 R²², and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

each R¹⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —XR²³, —C(═O)N(R¹⁵)₂, —(C₁₋₄ alkylene)ₚN(R²⁴)₂, —(C₁₋₄ alkylene)ₚheterocyclyl optionally substituted with 1-10 R²², and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚOR¹⁹, —(C₁₋₄ alkylene)ₚheterocyclyl optionally substituted with 1-10 R²², —(C₁₋₄ alkylene)ₚcarbocyclyl optionally substituted with 1-12 R²¹, —N(R¹⁵)(R²⁵), —C(═O)(R²⁶), —(C₁₋₄ alkylene)C(═O)OR²⁷, —(C₁₋₄ alkylene)aryl optionally substituted with one or more halides, —(C₁₋₄ alkylene)ₚheteroaryl optionally substituted with one or more halides, and —SO₂(R²⁸); wherein each —(C₁₋₄ alkylene) is, independently, optionally substituted with one or more substituents as defined anywhere herein;

alternatively, two R¹¹ attached to the same carbon atom can together represent ═O to form a carbonyl group;

each R¹² is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚOR¹⁹, —N(R¹⁵)(R²⁹), —C(═O)(R²⁶), —C(═O)OR²⁷, —(C₁₋₄ alkylene)ₚheterocyclyl optionally substituted with 1-10 R²², and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

R¹³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚheterocyclyl optionally substituted with 1-10 R²⁰, and -carbocyclyl optionally substituted with 1-12 R²¹; wherein —(C₁₋₄ alkylene) is, optionally substituted with one or more substituents as defined anywhere herein;

R¹⁴ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚheterocyclyl optionally substituted with 1-10 R²⁰, and -carbocyclyl optionally substituted with 1-12 R²¹; wherein —(C₁₋₄ alkylene) is, optionally substituted with one or more substituents as defined anywhere herein;

each R¹⁵ is selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

R¹⁶ is selected from the group consisting of —(C₁₋₄ alkylene)ₚheterocyclyl optionally substituted with 1-10 R²⁰, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

R¹⁷ is independently selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

alternatively, two adjacent R¹⁷ are taken together to form a -heterocyclyl ring optionally substituted with 1-10 R²²;

R¹⁸ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C═O)R¹⁵, and —(C₁₋₄ alkylene)ₚheterocyclyl optionally substituted with one or more halides or one or more unsubstituted —(C₁₋₅ alkyl); wherein —(C₁₋₄ alkylene) is, independently, optionally substituted with one or more substituents as defined anywhere herein;

each R¹⁹ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

each R²⁰ independently is selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —N(R¹⁵)₂, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

each R²¹ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN;

each R²² is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —N(R¹⁵)₂, —C(═O)R³⁴, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

each R²³ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)N(R¹⁵)₂, —(C₁₋₄ alkylene)ₚaryl optionally substituted with 1-10 R³⁰, —(C₁₋₄ alkylene)ₚheterocyclyl optionally substituted with 1-12 R³¹, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

each R²⁴ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)N(R¹⁵)₂; wherein each —(C₁₋₄ alkylene) is, independently, optionally substituted with one or more substituents as defined anywhere herein;

each R²⁵ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with 1-10 R³², —(C₁₋₄ alkylene)ₚcarbocyclyl optionally substituted with 1-12 R²¹, —(C₁₋₄ alkylene)OR³³; wherein each —(C₁₋₄ alkylene) is, independently, optionally substituted with one or more substituents as defined anywhere herein;

R²⁶ is selected from the group consisting of H, unsubstituted —(C₃₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or unsubstituted —(C₁₋₅ alkyl), —(C₁₋₄ alkylene)ₚheteroaryl optionally substituted with one or more halides or one or more unsubstituted —(C₁₋₅ alkyl), and —(C₁₋₄ alkylene)ₚheterocyclyl optionally substituted with one or more halides or one or more unsubstituted —(C₁₋₅ alkyl); wherein each —(C₁₋₄ alkylene) is, independently, optionally substituted with one or more substituents as defined anywhere herein;

R²⁷ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or one or more unsubstituted —(C₁₋₅ alkyl), —(C₁₋₄ alkylene)ₚheteroaryl optionally substituted with one or more halides or unsubstituted —(C₁₋₅ alkyl), and —(C₁₋₄ alkylene)ₚheterocyclyl optionally substituted with one or more halides or one or more unsubstituted —(C₁₋₅ alkyl); wherein each —(C₁₋₄ alkylene) is, independently, optionally substituted with one or more substituents as defined anywhere herein;

R²⁸ is selected from the group consisting of unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or one or more unsubstituted —(C₁₋₅ alkyl), —(C₁₋₄ alkylene)ₚheteroaryl optionally substituted with one or more halides or one or more unsubstituted —(C₁₋₅ alkyl), and —(C₁₋₄ alkylene)ₚheterocyclyl optionally substituted with one or more halides or one or more unsubstituted —(C₁₋₅ alkyl); wherein —(C₁₋₄ alkylene) is, optionally substituted with one or more substituents as defined anywhere herein;

each R²⁹ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with 1-10 R³², —(C₁₋₄ alkylene)ₚcarbocyclyl optionally substituted with 1-12 R²¹, —(C₁₋₄ alkylene)OR³³, and —C(═O)O(C₁₋₅ alkyl); wherein each —(C₁₋₄ alkylene) is, independently, optionally substituted with one or more substituents as defined anywhere herein;

each R³⁰ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN;

each R³¹ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —C(═O)R³⁴, —N(R²⁴)₂, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;

each R³² is independently selected from the group consisting of halide and unsubstituted —(C₁₋₅ alkyl);

each R³³ is independently selected from the group consisting of H and unsubstituted —(C₁₋₅ alkyl);

each R³⁴ is independently selected from the group consisting of —O(C₁₋₅ alkyl) and a heteroaryl optionally substituted with 1-6 R³⁵;

each R³⁵ is a -heterocyclyl optionally substituted with one or more halides or one or more unsubstituted —(C₁₋₅ alkyl);

each X is selected from the group consisting of O and S;

Y³ is CH or nitrogen;

Y¹, Y², Y⁴, and Y⁵ are independently selected from the group consisting of CH and nitrogen; wherein

if Y¹ is nitrogen then Y², Y⁴, and Y⁵ are carbon, Y³ is CH, and R⁴ is absent;

if Y² is nitrogen then Y¹, Y⁴, and Y⁵ are carbon, Y³ is CH, and R⁵ is absent;

if Y³ is nitrogen then Y¹, Y², Y⁴, and Y⁵ are carbon;

if Y⁴ is nitrogen then Y¹, Y², and Y⁵ are carbon, Y³ is CH, and R¹ is absent;

if Y⁵ is nitrogen then Y¹, Y², and Y⁴ are carbon, Y³ is CH, and R² is absent; and

each p is independently 0 or 1.

In some embodiments of any of the methods described herein, the dual CLK/DYRK inhibitor, CLK inhibitor, or DYRK inhibitor is a compound of Formula (VI):

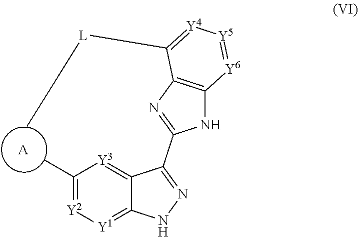

or a pharmaceutically acceptable salt or solvate thereof, wherein:

Ring A is a 5-6-membered heteroaryl optionally substituted with 1-4 R¹;

L is -L¹-L²-L³-L⁴-;

L¹ is selected from the group consisting of unsubstituted —(C₁₋₃ alkylene)-, —NR²—, —NR³(C═O)—, —(C═O)NR³—, and —O—;

L² is selected from the group consisting of unsubstituted —(C₁₋₆ alkylene)- and —NR²—;

L³ is selected from the group consisting of unsubstituted —(C₁₋₆ alkylene)-, —O—, and -carbocyclylene- optionally substituted with one or more halides;

L⁴ is selected from the group consisting of unsubstituted —(C₁₋₆ alkylene)-, —O—, —NR²—, —NR³(C═O)—, —(C═O)NR³—, -arylene- substituted with 1-5 R⁴, and -heteroarylene- optionally substituted with 1-4 R⁵;

with the proviso that —NR²— and —O— are not adjacent to each other;

with the proviso that two —NR²— and/or two —O— are not adjacent to each other;

with the proviso that two —NR³(C═O)— and/or —(C═O)NR³—, are not adjacent to each other;

each R¹ is selected from the group consisting of halide, unsubstituted —(C₁₋₃ alkyl), unsubstituted —(C₁₋₃ haloalkyl), and —CN;

each R² is selected from the group consisting of H and unsubstituted —(C₁₋₆ alkyl);

each R³ is selected from the group consisting of H and unsubstituted —(C₁₋₆ alkyl);

each R⁴ is selected from the group consisting of halide, unsubstituted —(C₁₋₆ alkyl), unsubstituted —(C₁₋₆ haloalkyl), and —CN;

each R⁵ is selected from the group consisting of halide, unsubstituted —(C₁₋₆ alkyl), unsubstituted —(C₁₋₆ haloalkyl), and —CN;

Y¹, Y², Y³, Y⁴, Y⁵, and Y⁶ are independently selected from the group consisting of CH and nitrogen; wherein

if Y¹ is nitrogen then Y² and Y³ are CH;

if Y² is nitrogen then Y¹ and Y³ are CH;

if Y³ is nitrogen then Y¹ and Y² are CH;

if Y⁴ is nitrogen then Y⁵ and Y⁶ are CH;

if Y⁵ is nitrogen then Y⁴ and Y⁶ are CH; and

if Y⁶ is nitrogen then Y⁴ and Y⁵ are CH.

In some embodiments of any of the methods described herein, the dual CLK/DYRK inhibitor, CLK inhibitor, or DYRK inhibitor is a compound of Formula (VII):

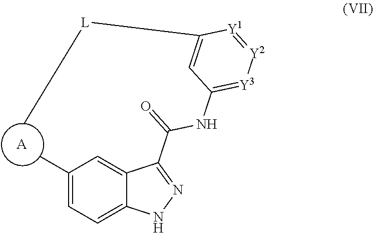

or a pharmaceutically acceptable salt or solvate thereof, wherein:

Ring A is a 5-6-membered heteroaryl optionally substituted with 1-3 R¹;

L is -L¹-L²-L³-L⁴-

L¹ is selected from the group consisting of unsubstituted —(C₁₋₃ alkylene)-, —NR²— —NR³(C═O)—, —(C═O)NR³—, and —O—;

L² is selected from the group consisting of unsubstituted —(C₁₋₆ alkylene)-, —NR²—, —NR³(C═O)—, and —(C═O)NR³—;

L³ is selected from the group consisting of unsubstituted —(C₁₋₆ alkylene)-, —O—, and carbocyclylene optionally substituted with one or more halides;

L⁴ is selected from the group consisting of unsubstituted —(C₁₋₆ alkylene)-, —O—, —NR²—, —NR³(C═O)—, —(C═O)NR³—, -arylene substituted with 1-5 R⁴, and -heteroarylene optionally substituted with 1-4 R⁵;

with the proviso that —NR²— and —O— are not adjacent to each other;

with the proviso that two —NR²— and/or two —O— are not adjacent to each other;

with the proviso that two —NR³(C═O)— and/or —(C═O)NR³—, are not adjacent to each other;

each R¹ is selected from the group consisting of halide, unsubstituted —(C₁₋₃ alkyl), unsubstituted —(C₁₋₃ haloalkyl), and —CN;

each R² is selected from the group consisting of H and unsubstituted —(C₁₋₆ alkyl);

each R³ is selected from the group consisting of H and unsubstituted —(C₁₋₆ alkyl);

each R⁴ is selected from the group consisting of halide, unsubstituted —(C₁₋₆ alkyl), unsubstituted —(C₁₋₆ haloalkyl), and —CN;

each R⁵ is selected from the group consisting of halide, unsubstituted —(C₁₋₆ alkyl), unsubstituted —(C₁₋₆ haloalkyl), and —CN;

Y¹, Y², and Y³ are independently selected from the group consisting of CH and nitrogen; wherein

if Y¹ is nitrogen then Y² and Y³ are CH;

if Y² is nitrogen then Y¹ and Y³ are CH; and

if Y³ is nitrogen then Y¹ and Y² are CH.

In some embodiments of any of the methods described herein, the dual CLK/DYRK inhibitor, CLK inhibitor, or DYRK inhibitor is a compound of Formula (VIII):

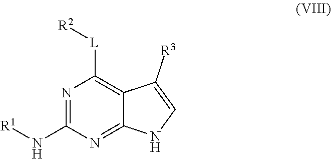

or a pharmaceutically acceptable salt or solvate thereof, wherein:

R¹ is selected from the group consisting of H, unsubstituted —(C₁₋₆ alkyl), unsubstituted —(C₂₋₆ alkenyl), unsubstituted —(C₂₋₆ alkynyl), and -heteroaryl optionally substituted with 1-4 R⁴, -aryl optionally substituted with 1-5 R⁵;

R² is selected from the group consisting of H, —(C₁₋₄ alkylene)ₚheteroaryl optionally substituted with 1-4 R⁶, —(C₁₋₄ alkylene)ₚheterocyclyl optionally substituted with 1-10 R⁷, and —(C₁₋₄ alkylene)ₚcarbocyclyl optionally substituted with 1-12 R⁸; wherein each —(C₁₋₄ alkylene) is, independently, optionally substituted with one or more substituents as defined anywhere herein;

R³ is selected from the group consisting of -heteroaryl optionally substituted with 1-4 R⁹ and -aryl optionally substituted with 1-5 R¹⁰;

each R⁴ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R¹⁴, and —(C₁₋₄ alkylene)ₚcarbocyclyl optionally substituted with 1-12 R¹⁵; wherein each —(C₁₋₄ alkylene) is, independently, optionally substituted with one or more substituents as defined anywhere herein;

each R⁵ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R⁴, and —(C₁₋₄ alkylene)ₚcarbocyclyl optionally substituted with 1-12 R⁵; wherein each —(C₁₋₄ alkylene) is, independently, optionally substituted with one or more substituents as defined anywhere herein;

each R⁶ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;

each R⁷ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁸ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁹ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;

each R¹⁰ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;

each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹² is independently selected from the group consisting of halide, unsubstituted —(C₁₋₆ alkyl), unsubstituted —(C₂₋₆ alkenyl), and unsubstituted —(C₂₋₆ alkynyl);

each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹⁴ is independently selected from the group consisting of halide, unsubstituted —(C₁₋₆ alkyl), unsubstituted —(C₂₋₆ alkenyl), and unsubstituted —(C₂₋₆ alkynyl);

each R¹⁵ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

L is selected from the group consisting of a bond, —O—, and —NH—; and

each p is independently 0 or 1.

Other features and advantages of the invention will be apparent from the following detailed description and figures, and from the claims.

### DESCRIPTION OF DRAWINGS

FIG. 1A depicts a dose-response curve or in vitro biochemical inhibition of CLK2, CLK3, and DYRK1A by compound 123 (n=4).

FIG. 1B depicts a Western Blot of phospho-SRSF in human mesenchymal stem cells (hMSCs) following treatment with compound 123 or DMSO for 1 hr.

FIG. 1C depicts a Western blot of phospho-SRSF in human chondrocytes following treatment with compound 123 or DMSO for 1 hr. Thus, compound 123 dose-dependently inhibited the CLK mediated phosphorylation of SRSF4, 5, 6 in hMSCs and human chondrocytes (FIGS. 1B and 1C).

FIG. 1D depicts representative immunofluorescence images of hMSCs treated with compound 123 or DMSO for 6 hrs. Cells were stained with phospho-SC35 antibody (green) or DAPI nuclear stain (blue). Spliceosome modulation by compound 123 (30 nM, 100 nM) was shown by enlargement of nuclear speckles in hMSCs and chondrocytes compared to DMSO.

FIG. 1E depicts a Western blot of phospho- and total FOXO1 in human chondrocytes following treatment with IL1-β and compound 123 or DMSO for 72 hrs.

FIG. 1F depicts representative immunofluorescence images of human chondrocytes treated with IL1-β and compound 123 or DMSO for 72 hrs. Cells were stained with anti-FOXO1 antibody (red) and DAPI nuclear stain (blue). Thus, compound 123 dose-dependently inhibited the phosphorylation of FOXO1 in hMSCs and chondrocytes in the presence of OA-related inflammatory cytokine IL-1β, with corresponding increases in total FOXO1 protein levels and nuclear localization, compared to DMSO.

FIG. 1G depicts a Western blot of phospho-SIRT1 (Ser27 and Ser47) and total SIRT1 in hMSCs following treatment with IL1-β and compound 123 or DMSO for 24 hrs. Compound 123 dose-dependently and more potently inhibited phosphorylation of SIRT1 (pSer27, pSer47) in hMSCs and chondrocytes in the presence of IL-1β, compared to DMSO.

All scale bars in FIG. 1 are 10 μm, and β-actin serves as a loading control for Western blots.

FIG. 2A depicts gene expression of Wnt pathway markers AXIN2, TCF7, TCF4 and CTNNB1 in hMSCs at 72 hrs following treatment with either non-targeted control siRNA or siRNA specific to CLK2. Fold change relative to siRNA control is shown (n=3, Mean±95% CI, *p<0.05, **p<0.01, ***p<0.01, t-test).

FIG. 2B depicts gene expression of Wnt pathway markers AXIN2, TCF7, TCF4 and CTNNB1 in hMSCs at 72 hrs following treatment with either non-targeted control siRNA or siRNA specific to DYRK1A. Fold change relative to siRNA control is shown (n=3, Mean±95% CI, *p<0.05, **p<0.01, ***p<0.01, t-test). In hMSCs, CLK2 or DYRK1A knockdowns inhibited Wnt pathway gene expression (AXIN2, TCF7 and TCF4) while small increases were observed in β-catenin (CTNNB1) expression, compared to non-targeted siRNA control.

FIG. 2C depicts the effects of treatment of hMSCs with siRNA specific to CLK2, DYRK1A, CLK2+DYRK1A, non-targeted control siRNA, DMSO, compound 123 on Wnt pathway gene expression at 72 hrs following treatment, measured using the Nanostring nCounter® gene array.

FIGS. 2D and 2E depict gene expression changes of selected genes from FIG. 2C by qRT-PCR (n=3, Mean±95% CI, *p<0.05, **p<0.01, ***p<0.001, one-way ANOVA). Knockdowns of CLK2, DYRK1A and CLK2+DYRK1A decreased the expression of 20, 22, and 18 genes and upregulated 9, 10, and 11 genes, respectively (Nanostring's nCounter® Vantage 3D™ Wnt Pathways panel; >2-fold change, p<0.05, FDR corrected) compared to siCtrl, while compound 123 downregulated 19 genes and upregulated 9 genes. Downregulation of AXIN2, TCF7, LRP5, BAMBI, NKD1, PAI-1, LRP6, FZD6, FZD7, PITX2, ERBB2, and CTGF gene expression and increased levels of Wnt pathway inhibitors SFRP2 and DACT1 were determined by qPCR.

FIGS. 3A, 3B, and 3C depict gene expression of chondrocyte markers COMP, SOX9 and RUNX1 and osteogenic marker RUNX2 in hMSCs following treatment with either non-targeted control siRNA or siRNA specific to (3A) β-catenin, (3B) LEF1 or TCF4, (3C) TCF7 for 72 hrs measured by qRT-PCR. Fold change relative to siRNA control is shown (n=3, Mean±95% CI, **p<0.01, t-test).

FIG. 3D depicts a Western blot of COMP, SOX9 and RUNX1 in hMSCs following treatment with either non-targeted control siRNA, siRNA specific to TCF7, compound 123, or DMSO for 72 hrs, with β-actin shown as a loading control. CLK1 and CLK4 knockdowns induced small, but significantly increased (4-6-fold, p<0.05) COMP expression. CLK1 knockdown induced 1.5-fold increased SOX9 expression while CLK3 or DYRK1A knockdowns had no significant effects on COMP, SOX9 and RUNX1 expression compared to siCtrl (see FIG. 4F).

FIG. 3E depicts representative immunofluorescence images of chondrocytes treated with either non-targeted control siRNA or siRNA specific to β-catenin, LEF1, TCF4, TCF7 or DMSO, compound 123 (10 nM) and stained with Rhodamine B at 7 days. TGFβ3 acts as a positive control for chondrocyte differentiation. The scale bars are 10 μm.

FIG. 3F depicts the quantification of the chondrocytes from FIG. 3E (n=3, Mean±95% CI, ***p<0.001, one-way ANOVA).

FIGS. 4A-4F depict gene expression of chondrocyte markers (A and D) COMP, (B and E) SOX9, and (C and F) RUNX1 in hMSCs at 72 hrs following treatment with either siRNA specific to CLK2 or non-targeted control siRNA measured by qRT-PCR and Western blot. compound 123 and TGFβ3 serve as positive controls for chondrocyte differentiation. Fold change relative to siRNA control is shown (n=3, Mean±95% CI, *p<0.05, **p<0.01, ***p<0.001, one-way ANOVA), β-actin serves as loading control. CLK2 knockdown in hMSCs increased early chondrocyte differentiation, with 50-fold increased COMP, and 3-4-fold increased SOX9 and RUNX1 expression compared with siCtrl, and these changes were similar to TGFβ3 or treatment with compound 123.

FIG. 4G depicts the effects of compound 123 on chondrocyte gene expression in hMSCs at 21 days following treatment with either siRNA specific to CLK2, DYRK1A, CLK2+DYRK1A or non-targeted control siRNA, measured using the Nanostring nCounter® gene array. compound 123 serves as a positive control for chondrocyte differentiation.

FIG. 4H depicts the gene expression changes of selected genes from FIG. 4G by qRT-PCR (n=3, Mean±95% CI, *p<0.05, **p<0.01, ***p<0.001, one-way ANOVA). CLK2 knockdown induced expression of 10 chondrogenic genes including ACAN, CD44, COL2A1, DOTL1, and COMP, while DYRK1A knockdown alone induced upregulation of only GDF5 (>2-fold, p<0.05; Nanostring's nCounter® Vantage 3D™ chondrocyte panel) on day 21. CLK2+DYRK1A knockdown upregulated 15 genes including FGFR2, FOXO1, MAPK8, PRG4, and TGFβ3, in addition to ACAN, CD44, COL2A1, DOTL1 and COMP. Expression of CCNG2, CD44, COL2A1, COMP, DOTL1, TGFβ1 and TGFβ3 was significantly increased with CLK2+DYRK1A knockdown, compared to CLK2 knockdown alone, indicating a role for DYRK1A inhibition in enhancing the effects of CLK2 inhibition, or for maintenance of chondrocyte function.

FIG. 4I depicts gene expression of matrix metalloproteinases (MMP-1, MMP-3, MMP-13) in chondrocytes stimulated with IL-1β and treated with DMSO, compound 123, Harmine, or CC-671 measured by qRT-PCR. Fold change relative to unstimulated control is shown (n=3, Mean±95% CI, *p<0.05, **p<0.01, ***p<0.001, one-way ANOVA).

FIG. 5A depicts expression of phospho- and total NF-κB and STAT3 in synovial fibroblasts stimulated with LPS and treated with DMSO or compound 123 for 20 hrs measured by Western blot, with β-actin as a loading control.

FIG. 5B depicts expression of phospho- and total NF-κB and STAT3 in PBMCs stimulated with LPS and treated with DMSO or compound 123 for 20 hrs measured by Western blot, with β-actin as a loading control.

FIG. 5C depicts inhibition of IL-6 and TNF-α production in synovial fibroblasts stimulated with IL1-β and treated with DMSO or compound 123 for 24 hrs measured by HTRF and ELISA (n=3, Mean±SEM).

FIG. 5D depicts inhibition of IL-6 and TNF-α production in synovial fibroblasts stimulated with LPS and treated with DMSO or compound 123 for 24 hrs measured by HTRF and ELISA (n=3, Mean±SEM).

FIG. 5E depicts the production of pro-inflammatory cytokines in synovial fibroblasts stimulated with LPS and treated with DMSO or compound 123 for 72 hrs measured by MSD-based ELISA (n=3, Mean±95% CI, *p<0.05, **p<0.01, ***p<0.001, t-test).

FIG. 5F depicts the production of pro-inflammatory cytokines in PBMCs stimulated with LPS and treated with DMSO or compound 123 for 72 hrs measured by MSD-based ELISA (n=3, Mean±95% CI, *p<0.05, **p<0.01, ***p<0.001, t-test).

FIG. 5G depicts a Western blot of phospho- and total SRSF in synovial fibroblasts following treatment with compound 123 or DMSO for 1 hr, with β-actin as a loading control.

FIG. 5H depicts a Western blot of phospho- and total SRSF in (PBMCs following treatment with compound 123 or DMSO for 1 hr, with β-actin as a loading control.

FIG. 6A depicts gene expression of IL-6, TNF-α, IL-8 and IL1-β in BEAS-2B cells treated with siRNA specific to CLK2, DYRK1A, CLK2+DYRK1A, or compound 123 or non-targeted control siRNA and stimulated with LPS for 6 hrs measured by qRT-PCR. Fold change relative to unstimulated siRNA control is shown (n=3, Mean±95% CI, *p<0.05, **p<0.01, ***p<0.001, one-way ANOVA).

FIG. 6B depicts IL-6 and IL-8 protein production in BEAS-2B cells treated with siRNA specific to CLK2, DYRK1A, CLK2+DYRK1A, or compound 123 or non-targeted control siRNA and stimulated with LPS for 6 hrs measured by HTRF-based assay (n=3, Mean±95% CI, *p<0.05, **p<0.01, ***p<0.001, one-way ANOVA). Combined CLK2 and DYRK1A knockdown significantly decreased IL-6, IL-8, TNF-α and IL-1β gene expression and IL-6 and IL-8 protein compared to siCtrl or CLK2 knockdown

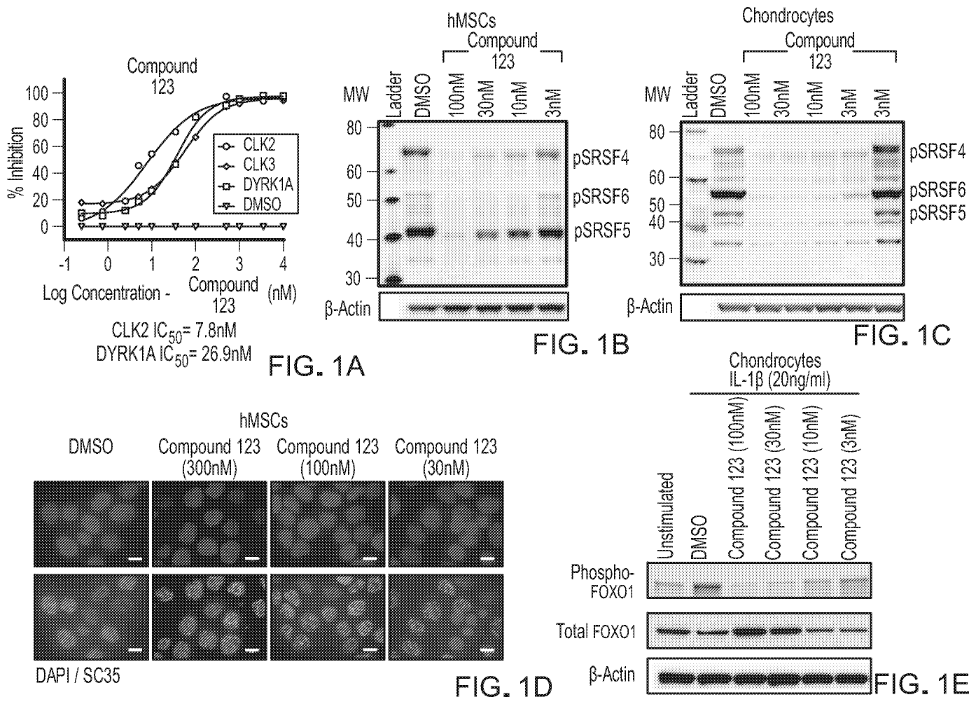

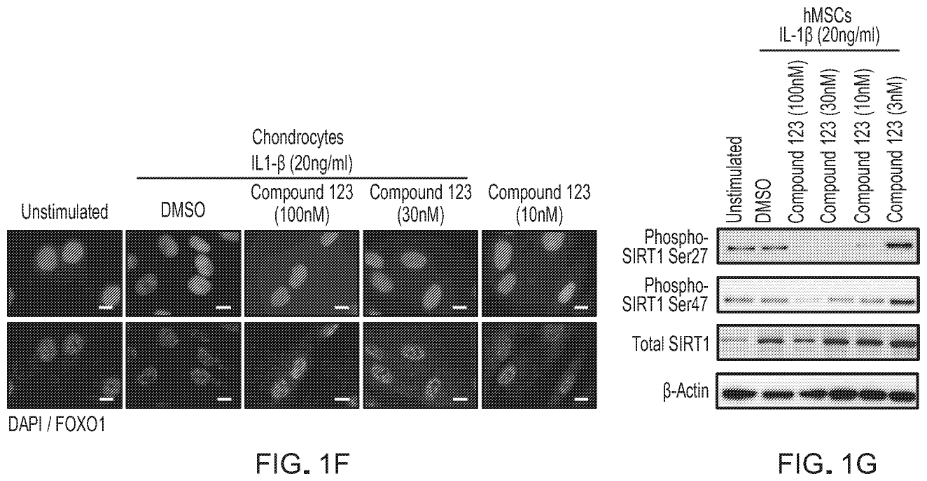

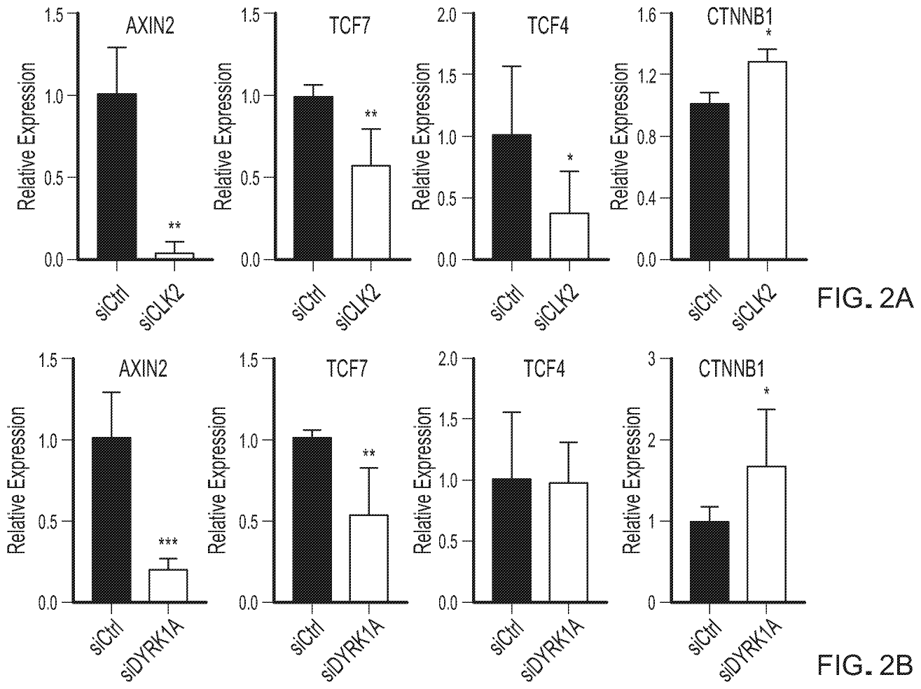

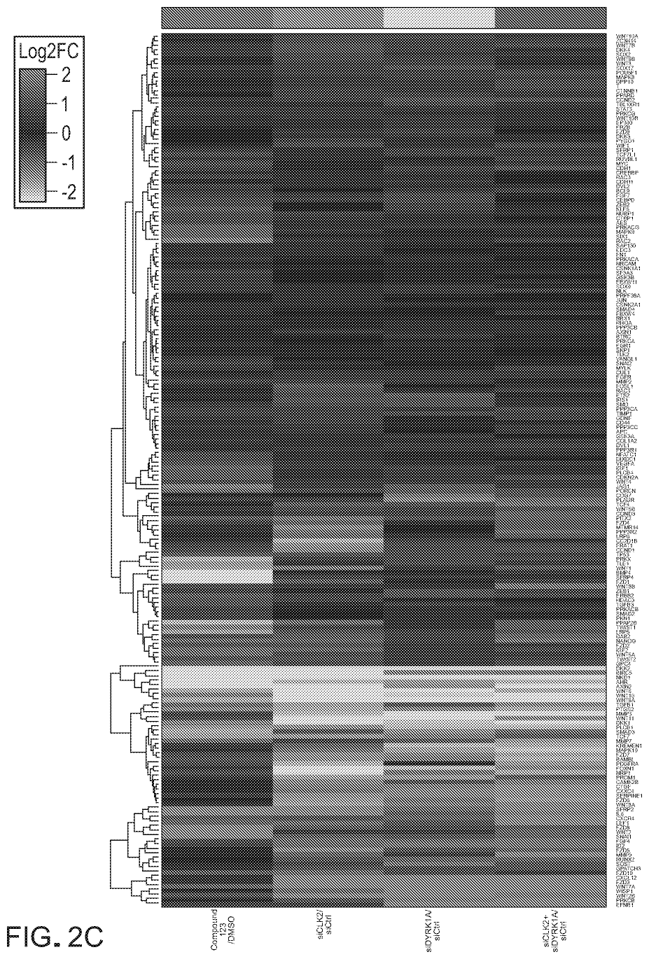

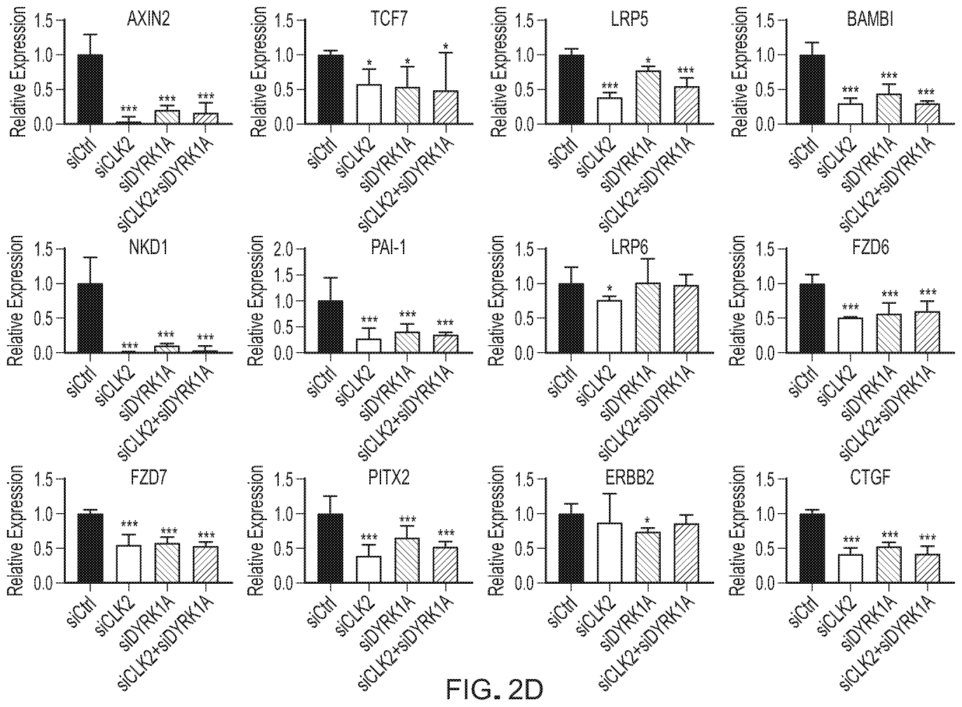

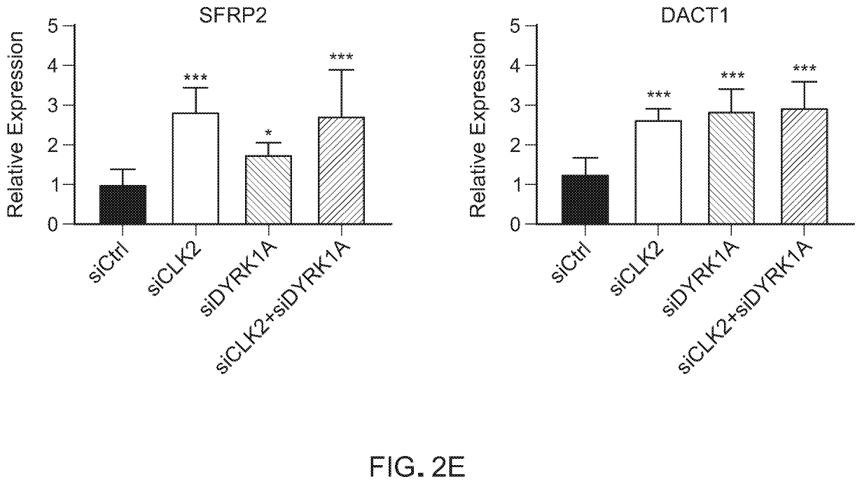

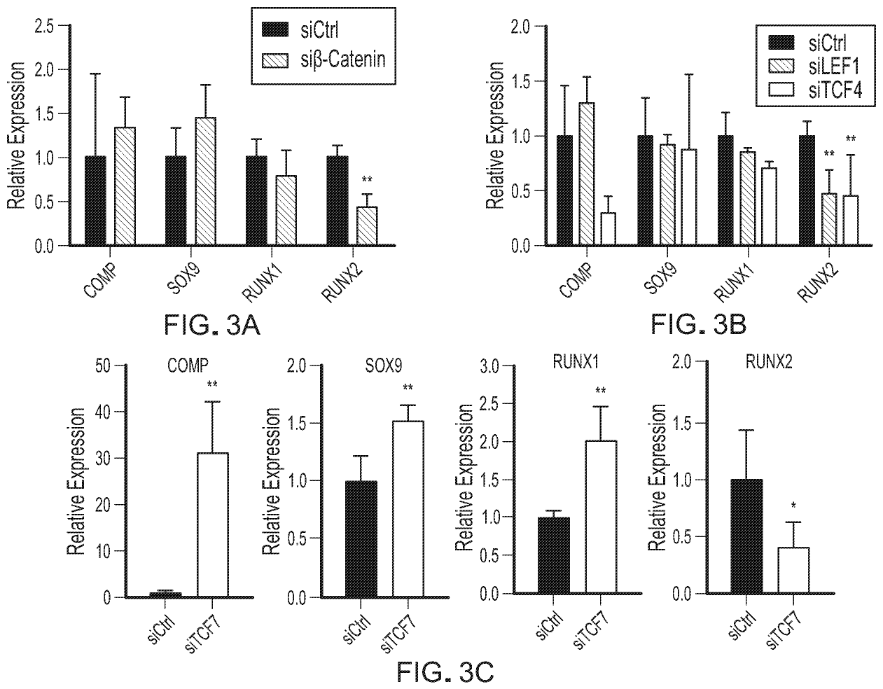

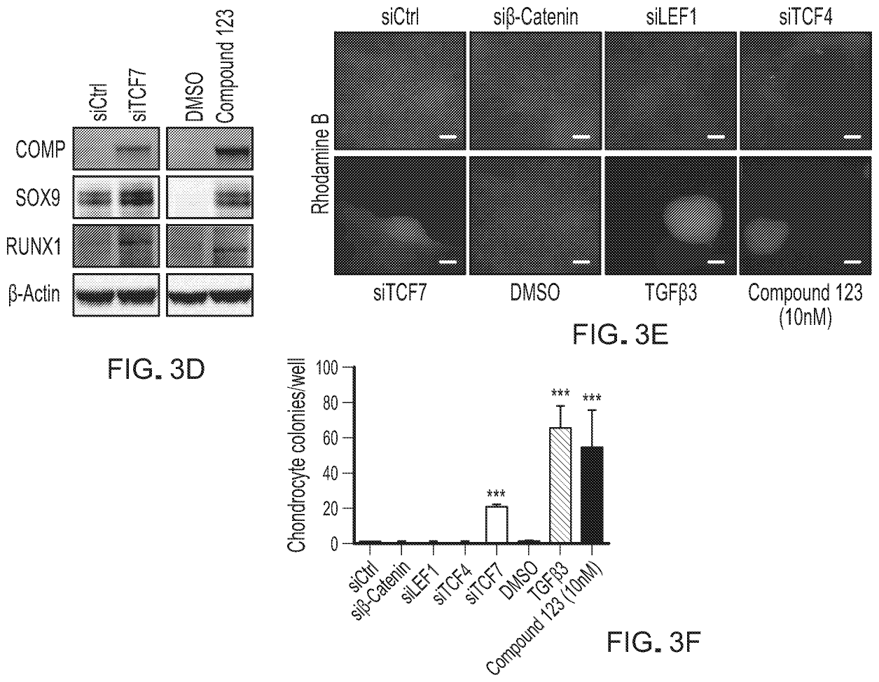

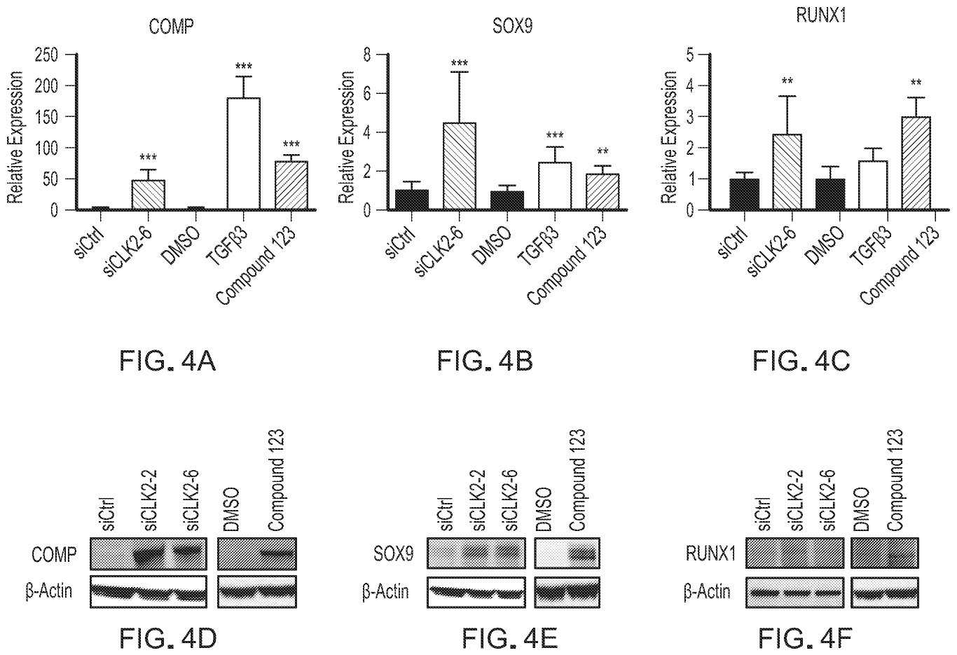

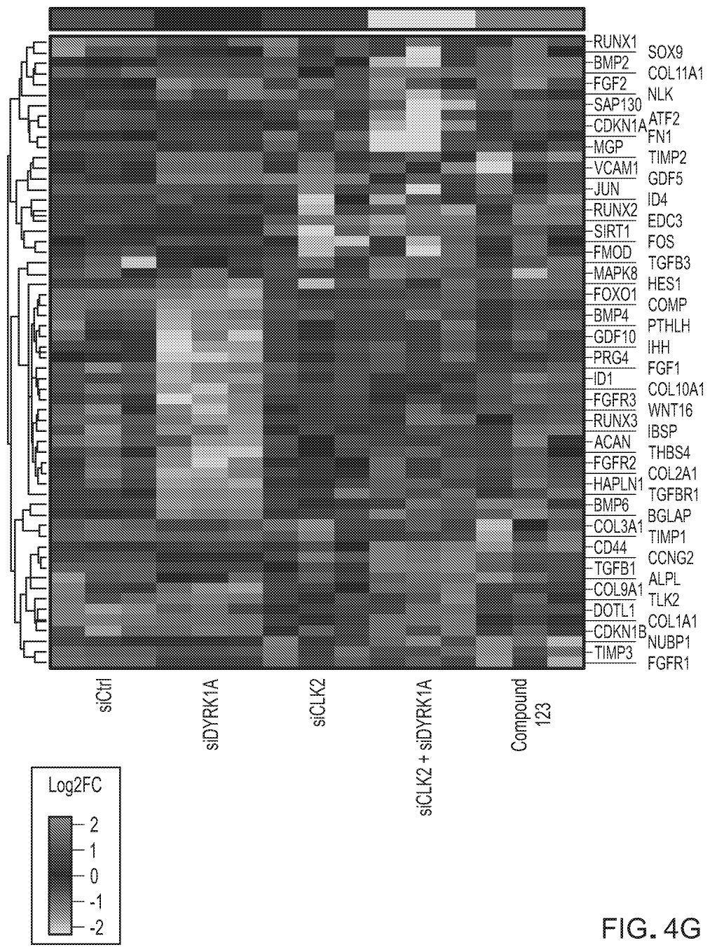

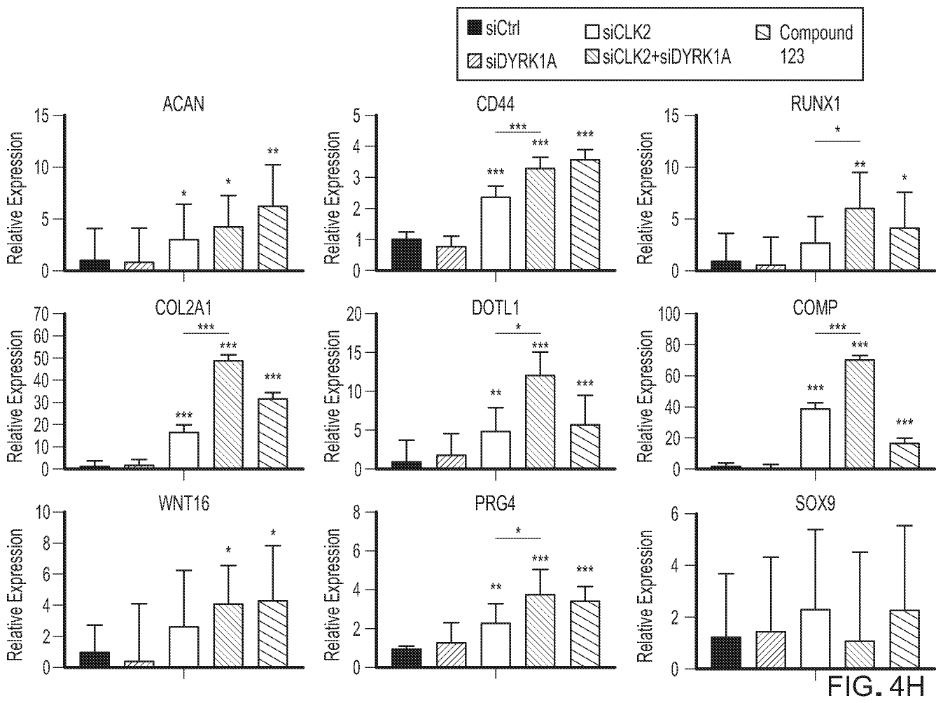

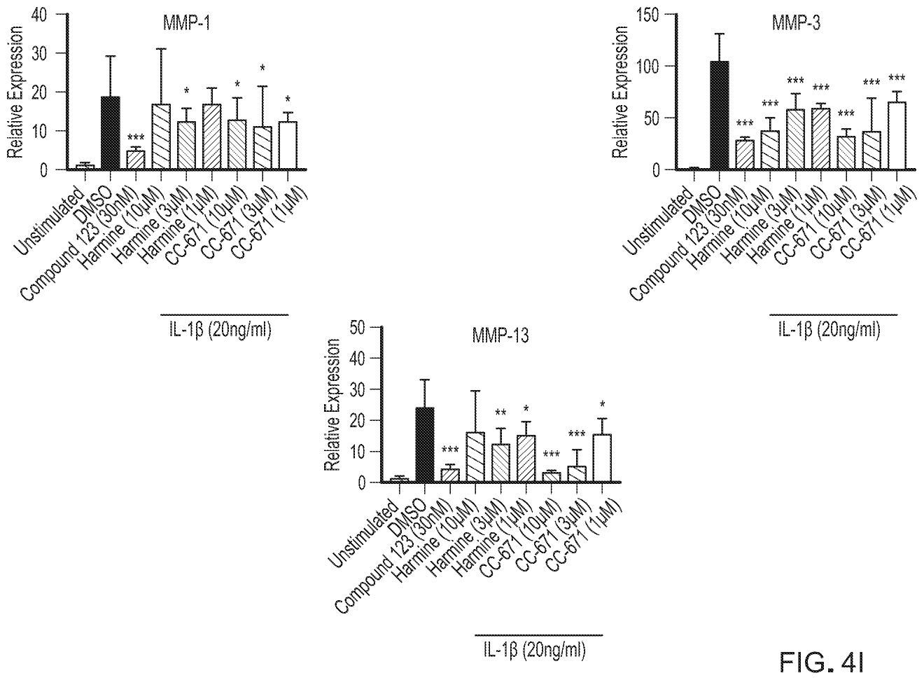

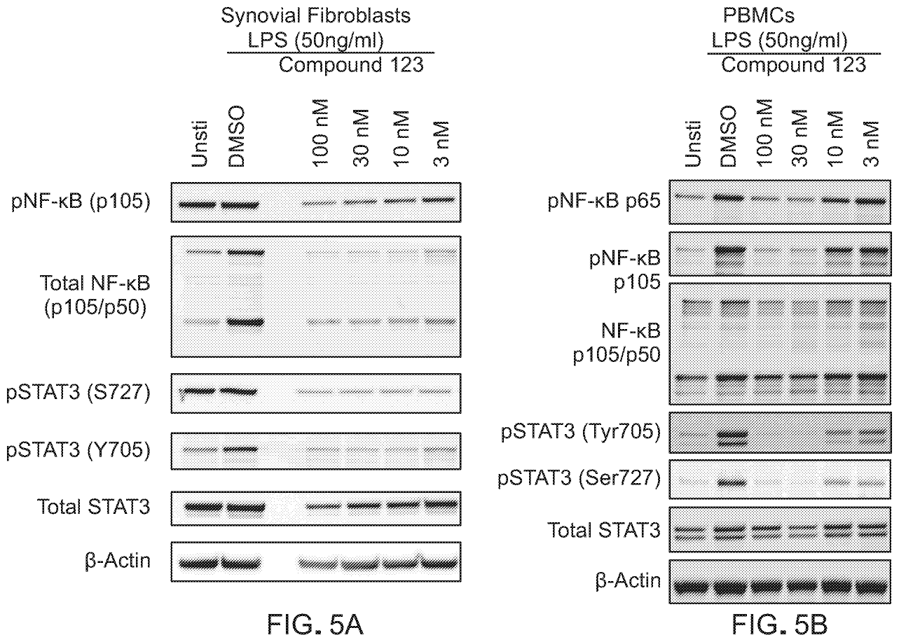

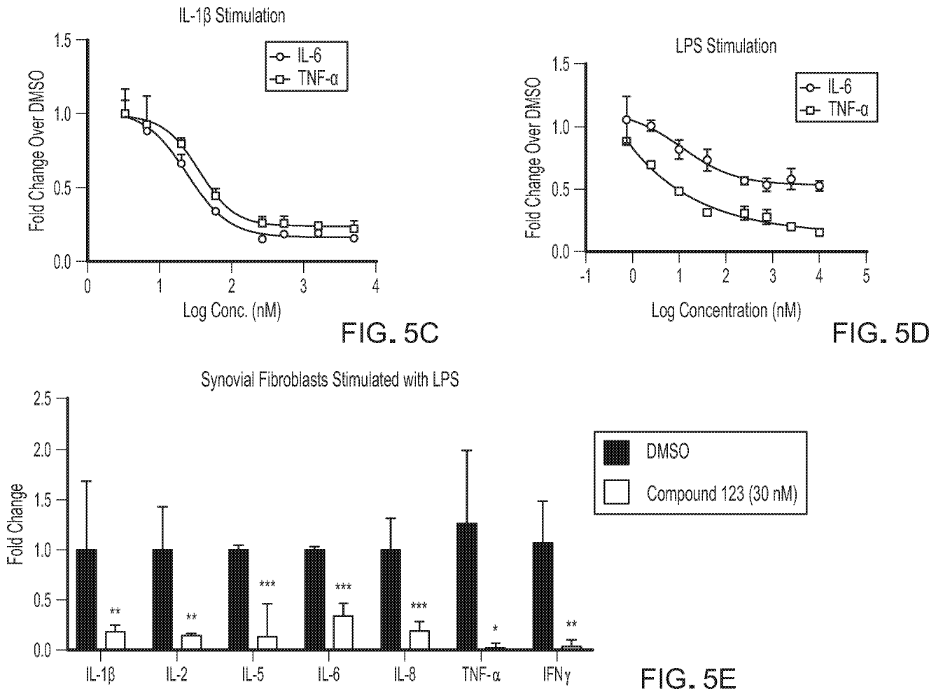

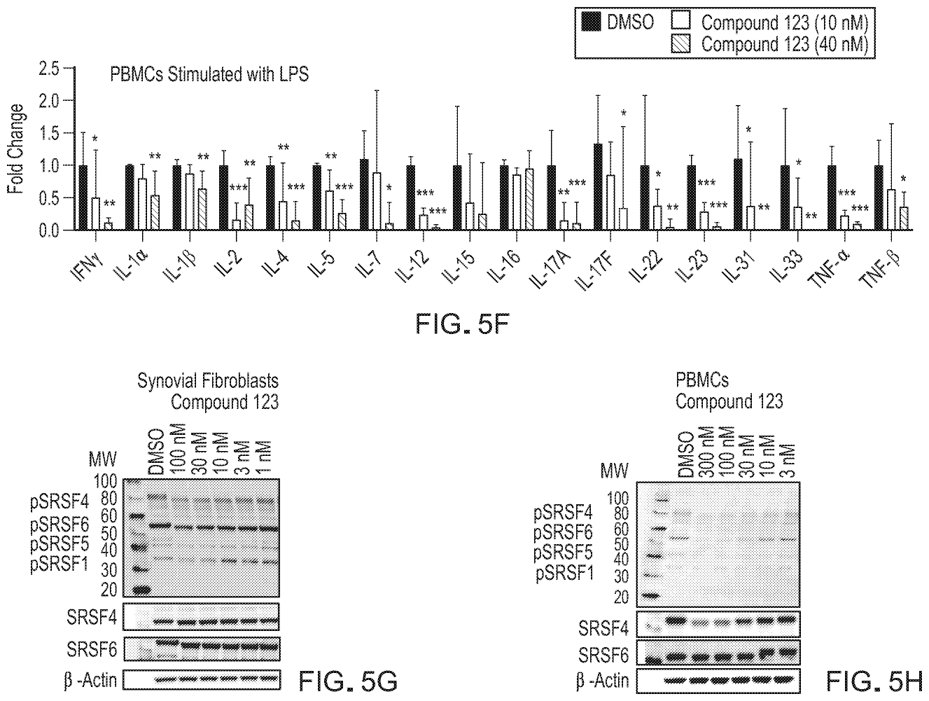

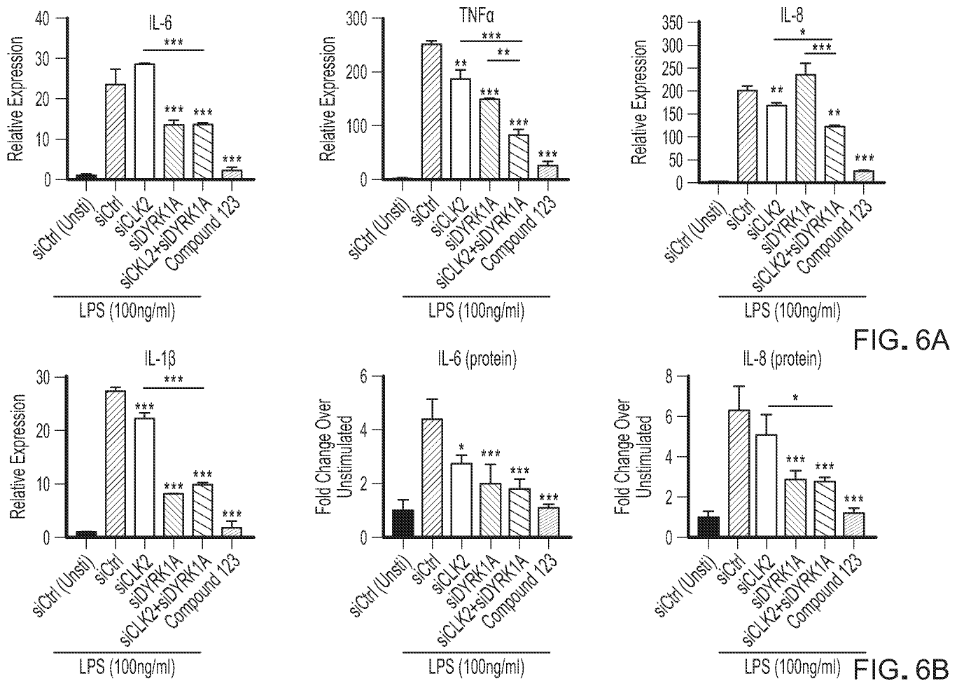

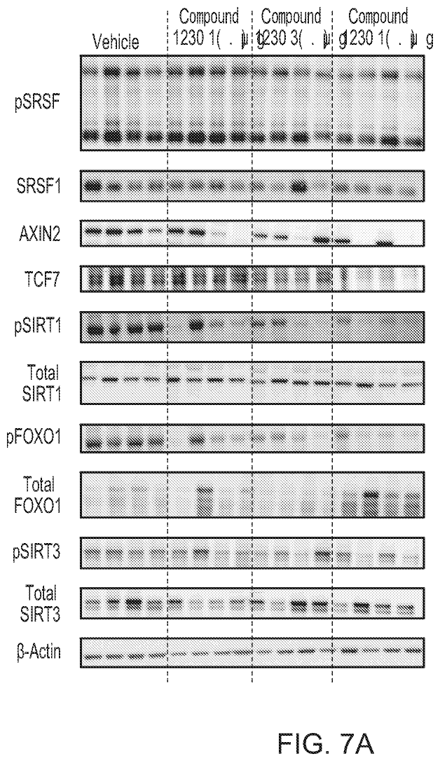

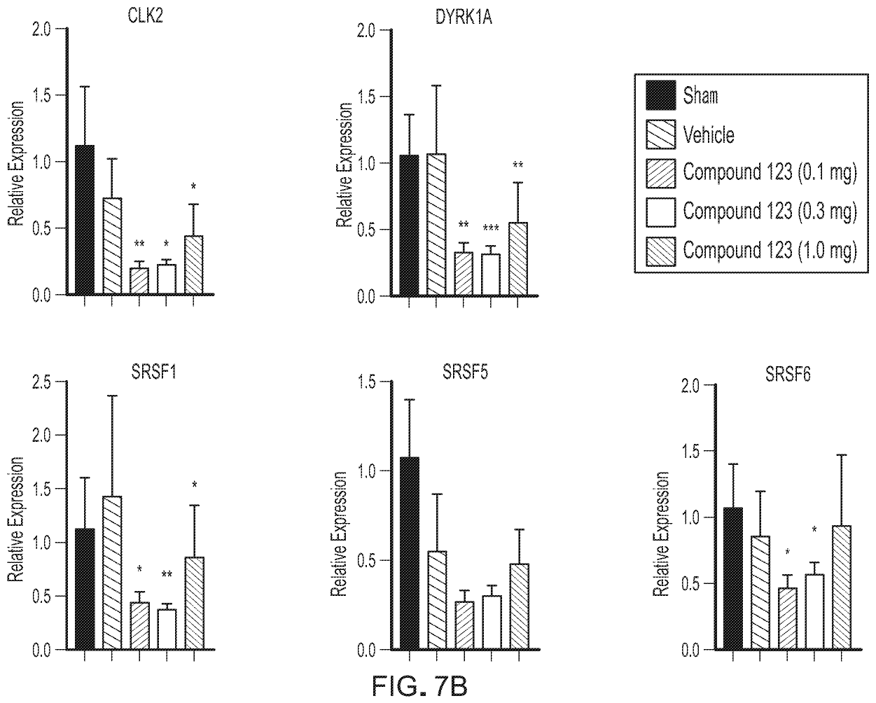

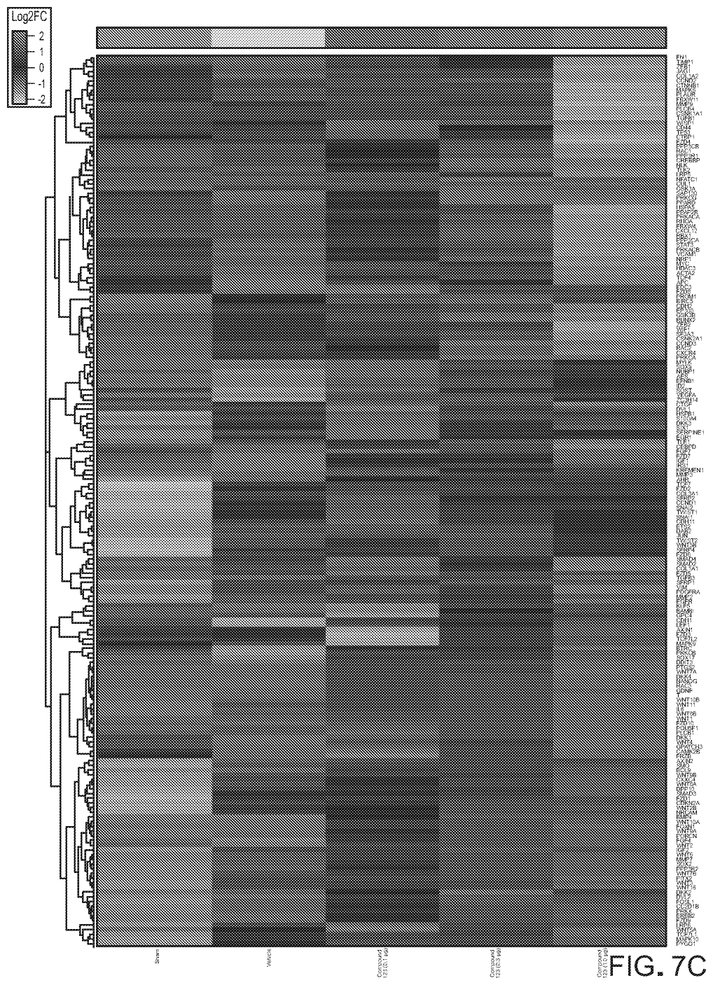

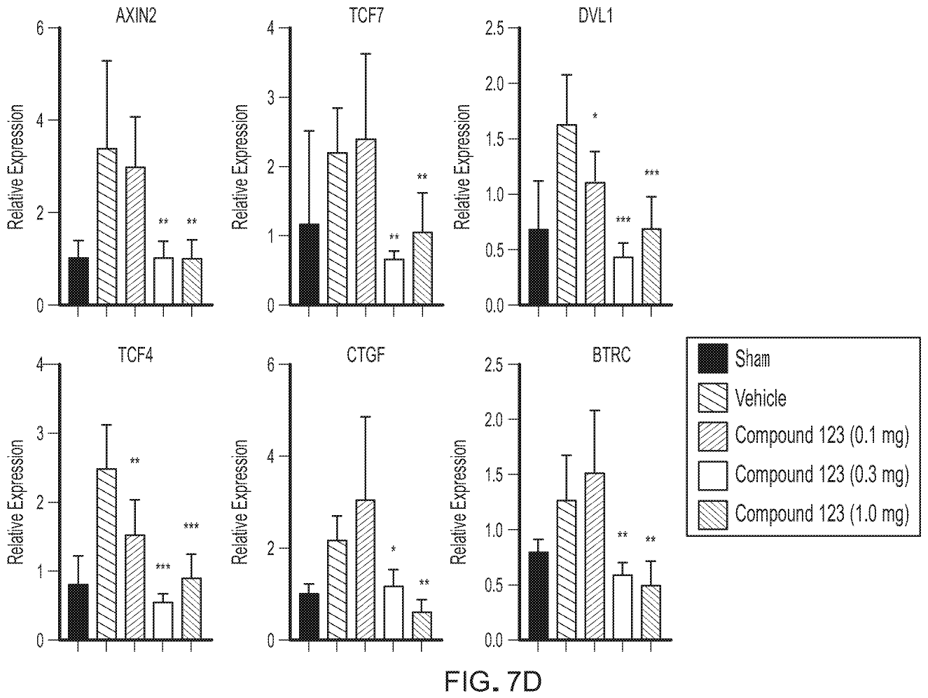

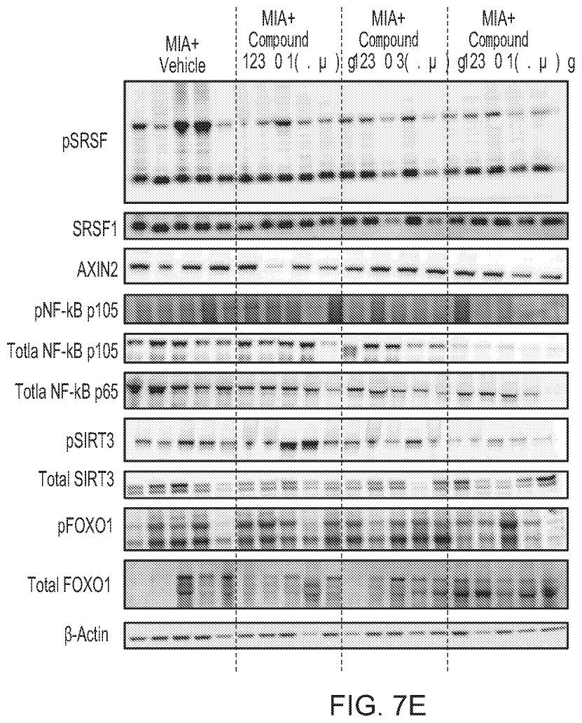

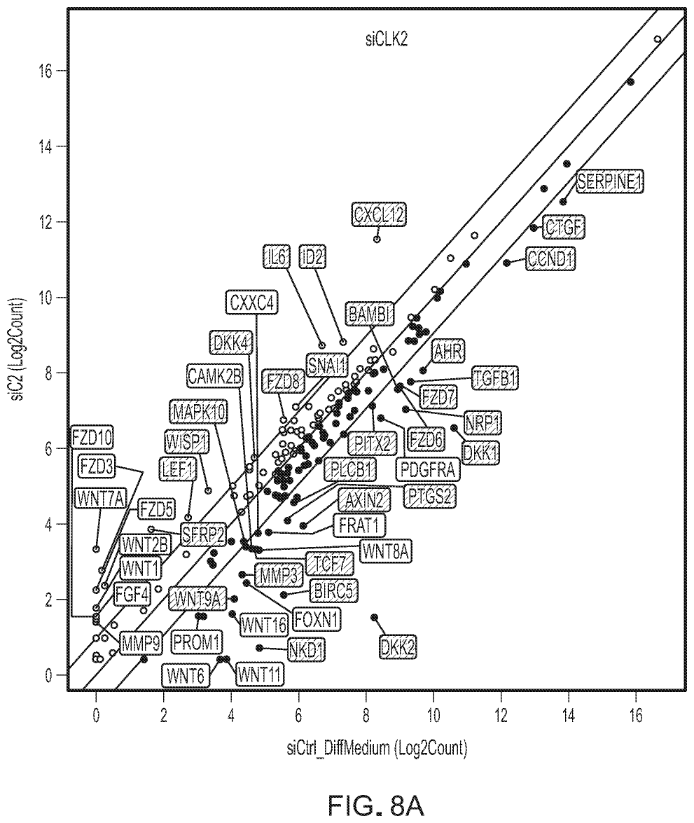

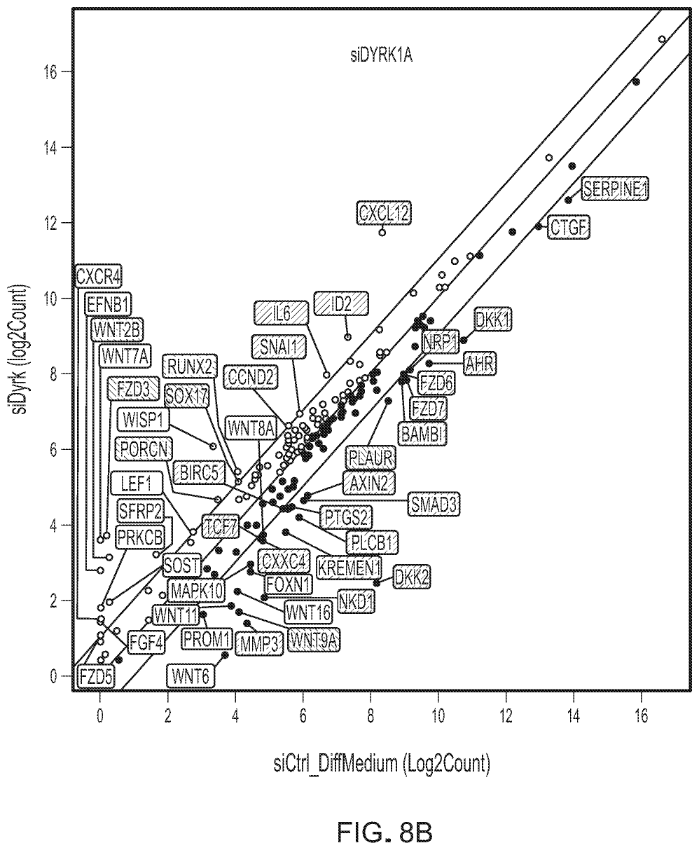

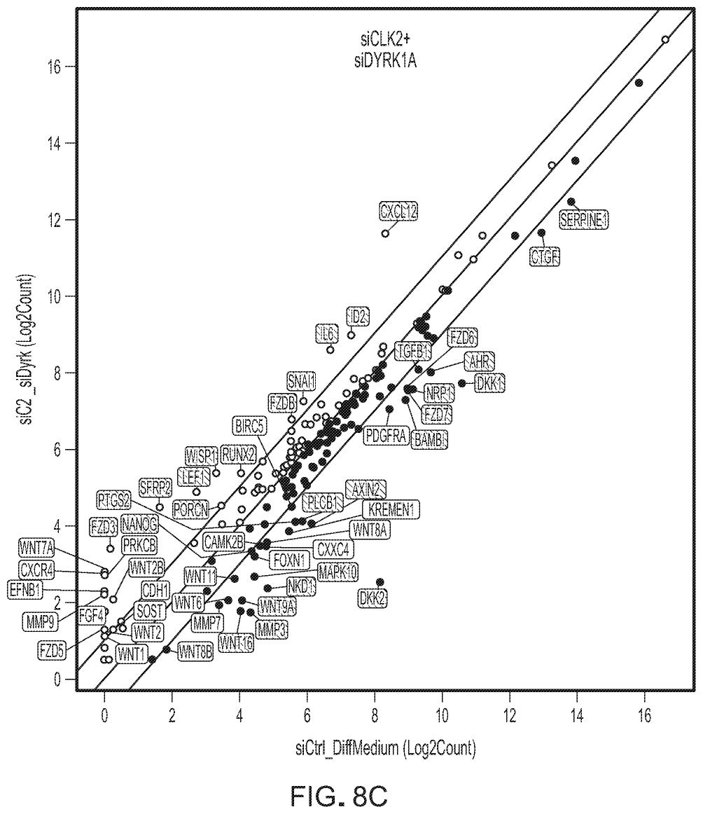

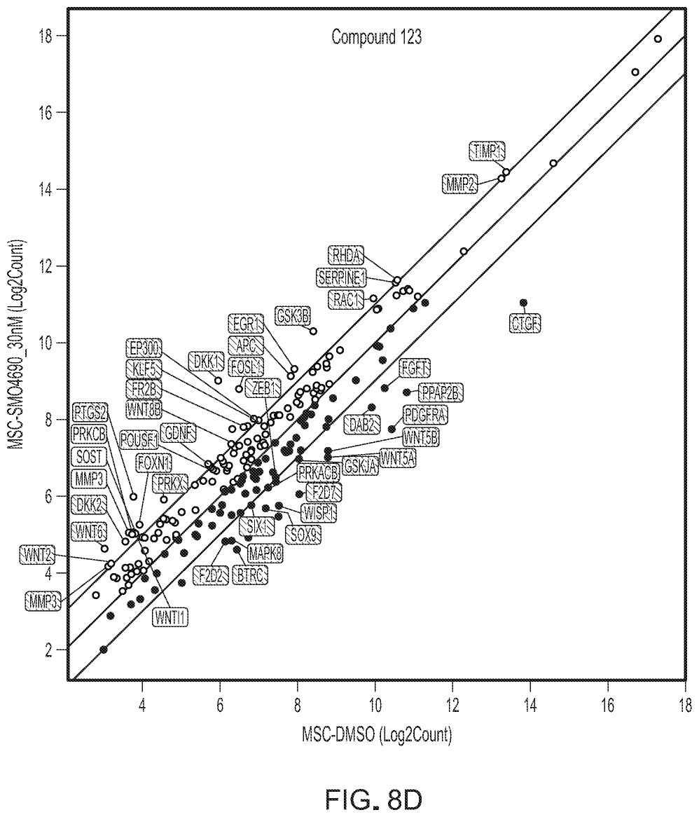

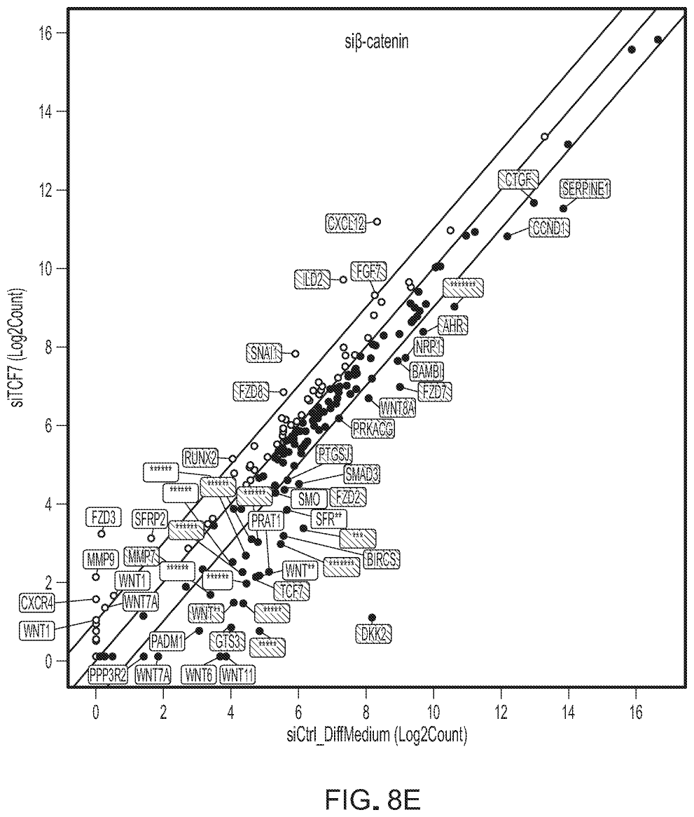

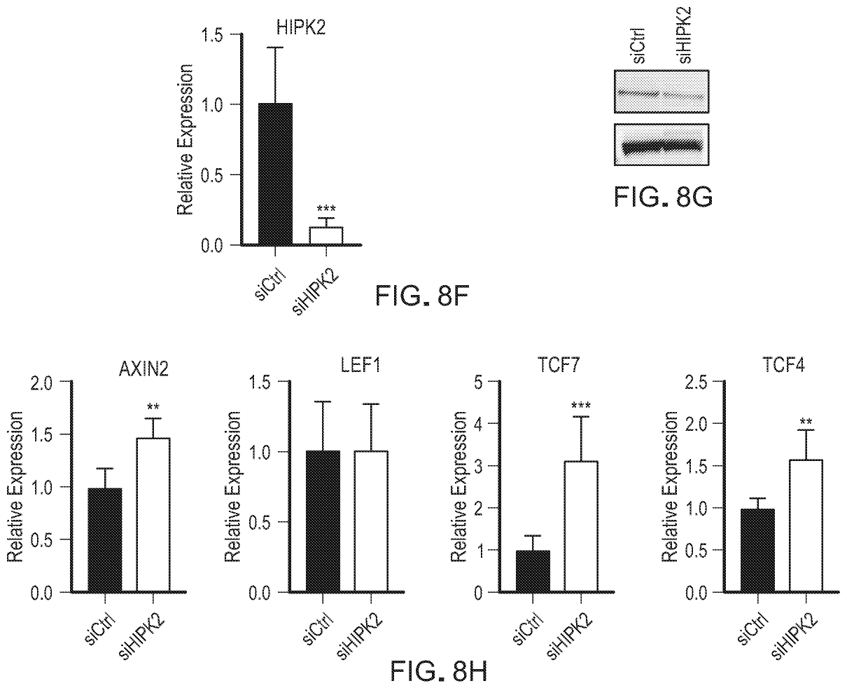

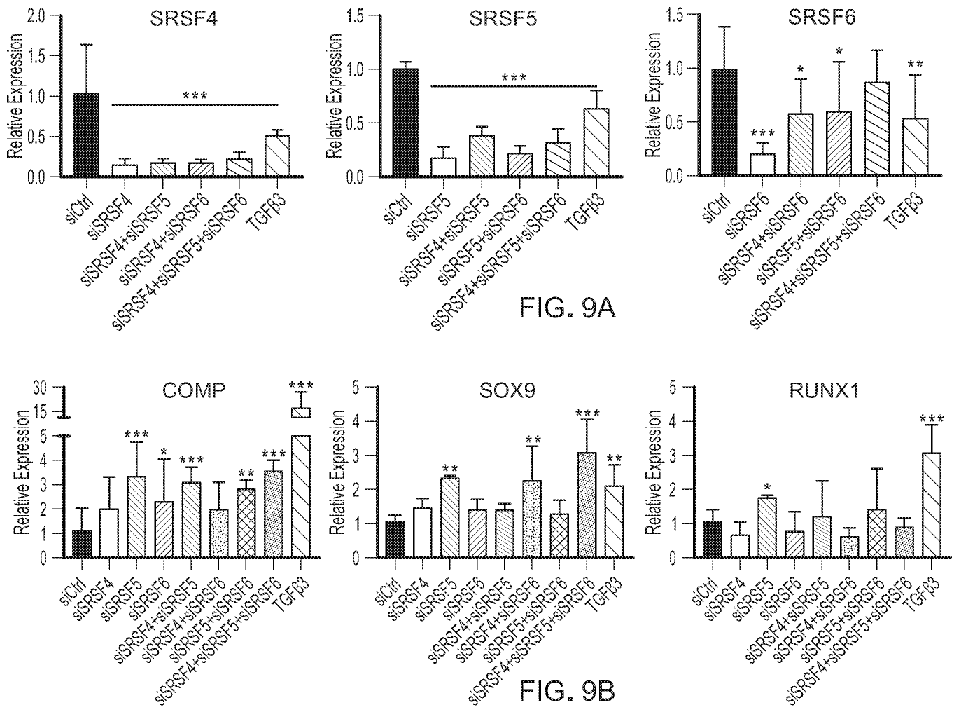

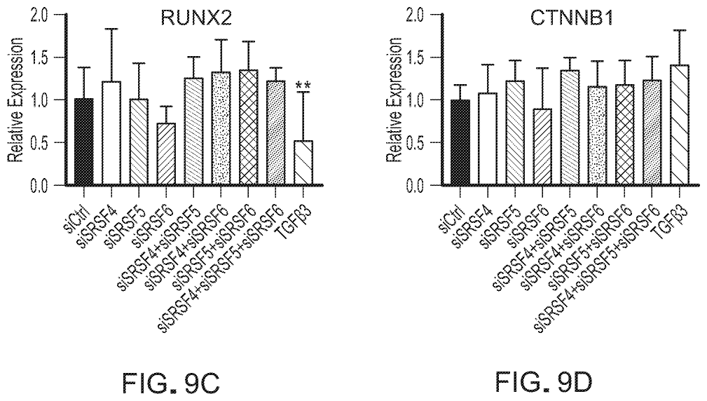

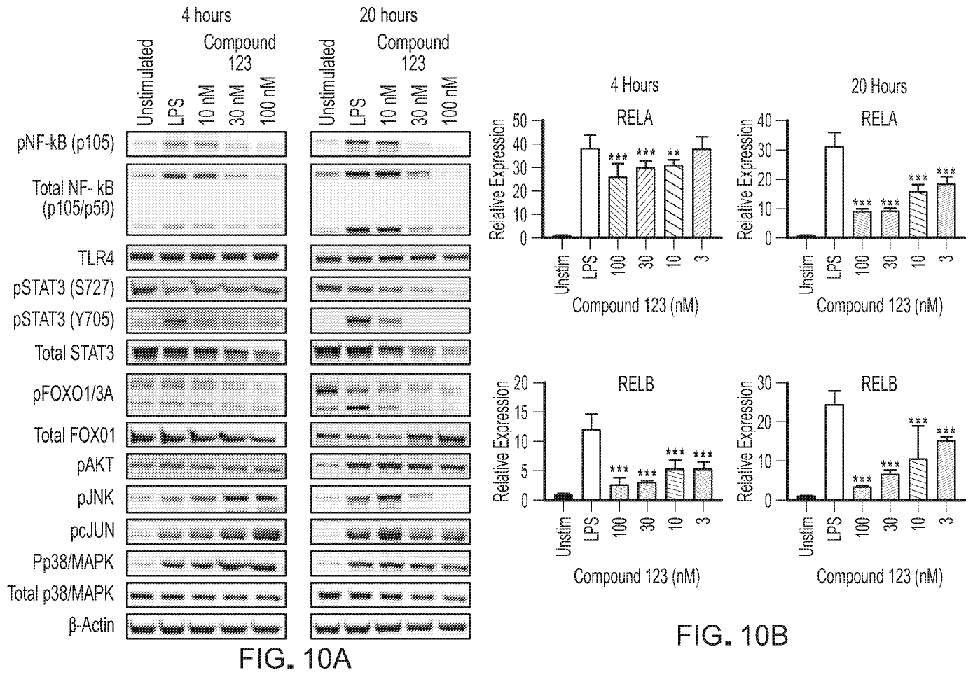

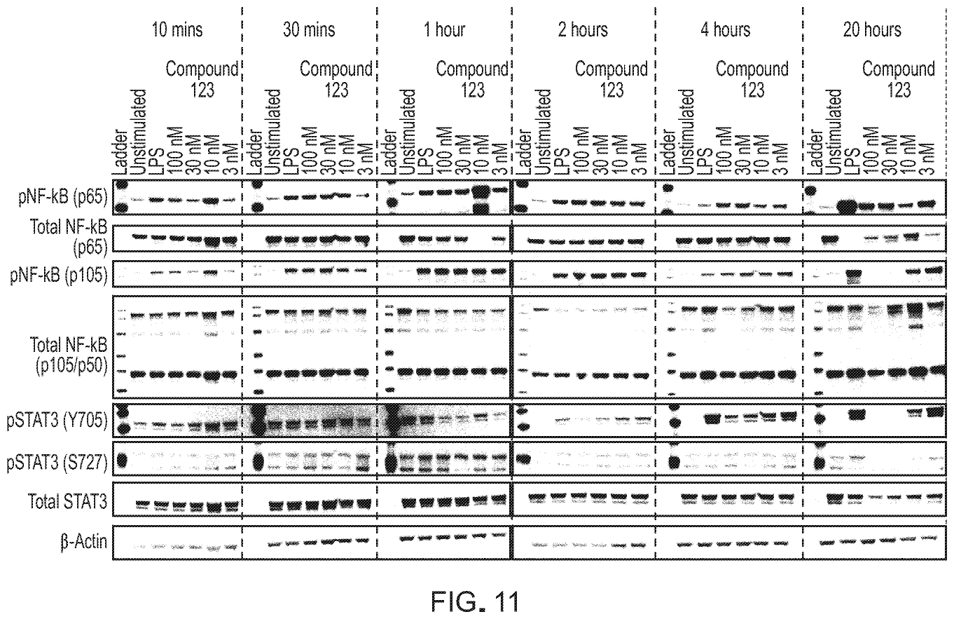

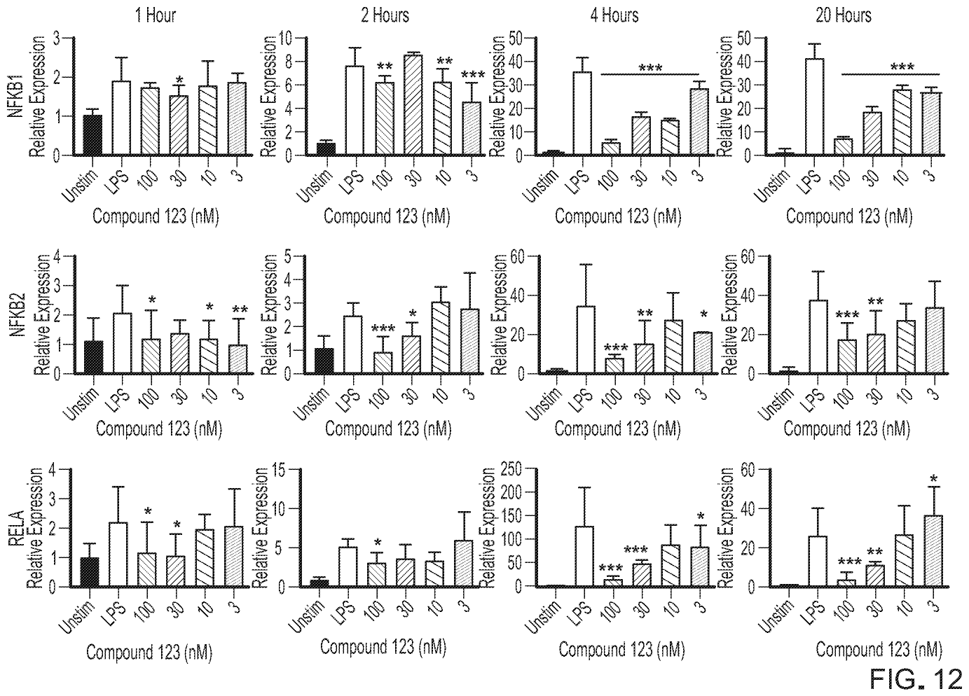

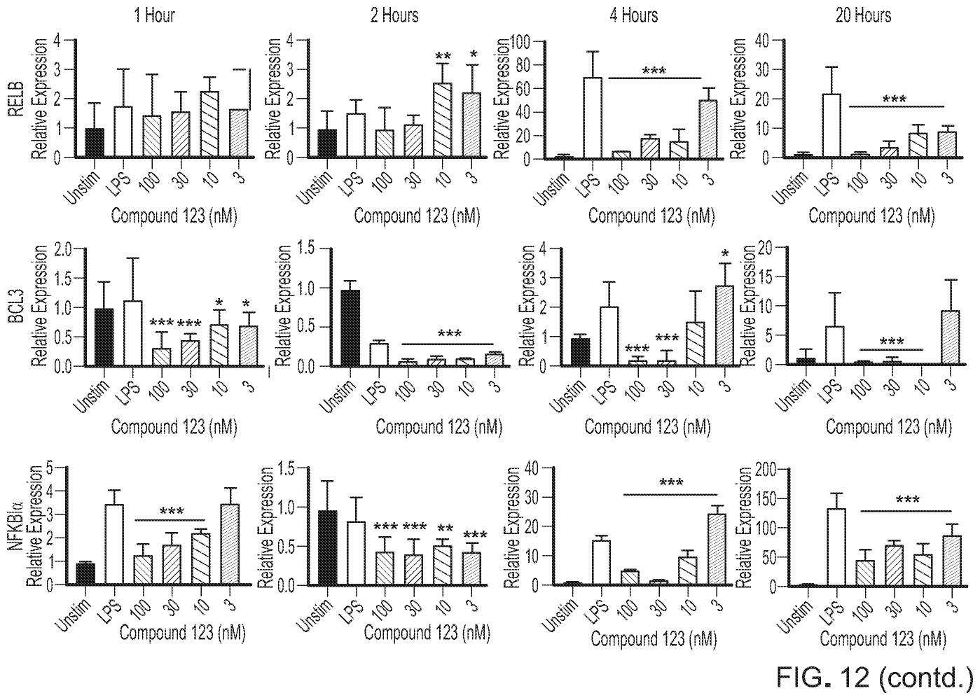

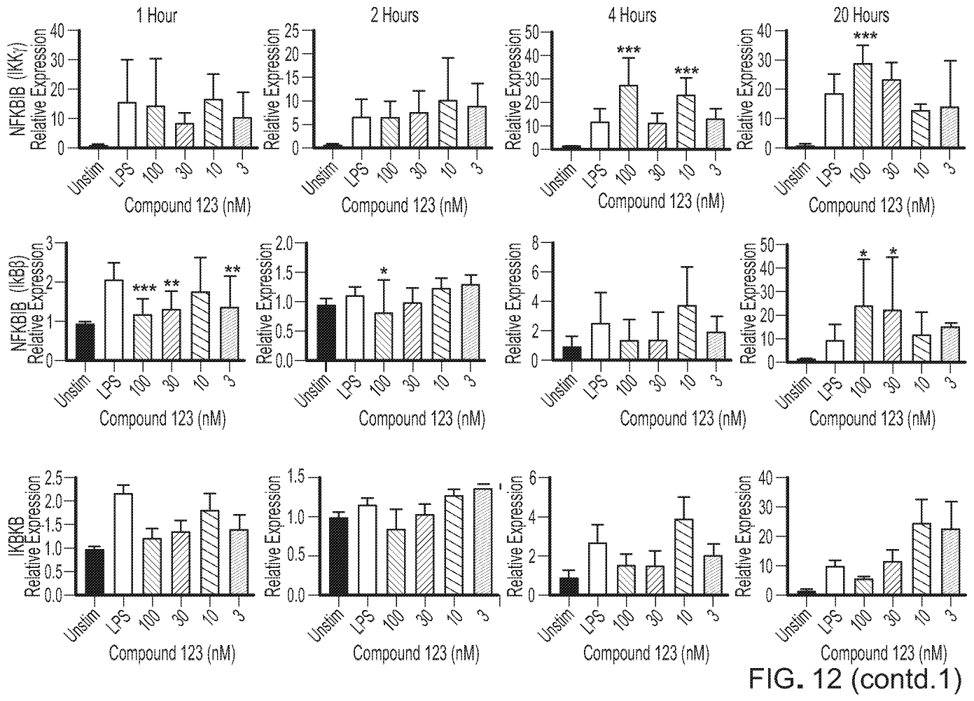

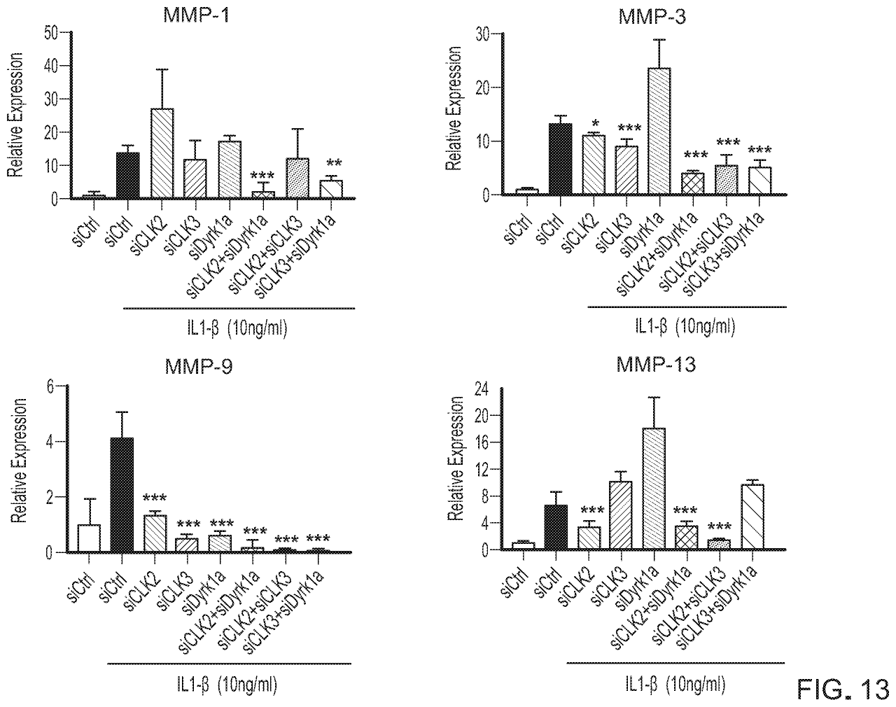

In FIG. 7A-7E, ACLT+pMMx rats were treated with an intra-articular injection of either vehicle or compound 123 (0.1 μg, 0.3 μg, 1.0 μg) 1 week after surgery and the cartilage was isolated at day 35. In the ACLT+pMMx and MIA models of OA, compound 123 (single IA injection—0.1 μg, 0.3 μg, 1 μg) one-week post ACLT+pMMx decreased phospho-SRSF, SRSF1, AXIN2, TCF7, phospho-SIRT1, phospho-FOXO1, and phospho-STAT3 compared with vehicle at day 35, and decreased CLK2, DYRK1A, SRSF1, SRSF5, and SRSF6 expression compared with vehicle. Compound 123 decreased expression of 19 genes and increased expression of at least 8 genes (including AXIN2, TCF7, DVL1, TCF4, CTGF and BTRC).

FIG. 7A depicts Western blots for phospho-SRSF, SRSF1, AXIN2, TCF7, phospho-SIRT1, total SIRT1, phospho-FOXO1, total FOXO1, phospho-STAT3 and total STAT3 in the cartilage, with β-actin as a loading control.

FIG. 7B depicts eene expression of CLK2, DYRK1A, SRSF1, 5 and 6 in cartilage measured by qRT-PCR (n=8, Mean±95% CI, *p<0.05, **p<0.01, ***p<0.001, one-way ANOVA).

FIG. 7C depicts Wnt pathway gene expression in cartilage, measured using the Nanostring nCounter® gene array.

FIG. 7D depicts gene expression changes of selected genes from FIG. 7C by qRT-PCR (n=8, Mean±95% CI, *p<0.05, **p<0.01, ***p<0.001, one-way ANOVA).

FIG. 7E depicts monoiodoacetate (MIA) injected rats treated with intra-articular injection of either vehicle or compound 123 (0.1 μg, 0.3 μg, 1.0 μg) and cartilage isolated at day 11. Western blots for phospho-SRSF, SRSF1, AXIN2, phospho-NF-κB, total NFκB, phospho-STAT3, total STAT3, phospho-FOXO1 and total FOXO1 in the cartilage, with β-actin as a loading control. In the MIA model, 4 days after single IA injection, compound 123 decreased phosphorylation of SRSF, NF-κB, STAT3, and FOXO1 compared to vehicle. While no changes in the total protein levels of SRSF1 and STAT3 were observed, compound 123 decreased protein levels of total NF-κB and increased protein levels of total FOXO1.

FIGS. 8A-8E depict the effects of treatment of hMSCs with siRNA specific to (FIG. 8A) CLK2 (72 hrs), (FIG. 8B) DYRK1A (72 hrs), eCLK2+DYRK1A (72 hrs), (FIG. 8D) compound 123 (24 hrs) or DMSO (24 hrs) or (FIG. 8E) β-catenin (72 hrs) or non-targeted control siRNA (72 hrs) on Wnt pathway gene expression, measured using the Nanostring nCounter® gene array (n=3, genes with >2-fold expression change, *p<0.05, FDR-adjusted are highlighted in green). Compared to DMSO, compound 123 (4 hrs) dose-dependently decreased phospho-STAT3 (S727, Y705), phospho- and total NF-κB (p105/p50), and phospho-FOXO1/3a, while AKT, JNK1, cJUN, p38/MAPK and TLR4 were not inhibited. Compound 123 (20 hrs) robustly inhibited NF-κB, STAT3, JNK1 and FOXO1/3a phosphorylation, while AKT, cJUN, p38/MAPK, and TLR4 remained unchanged (see also, FIG. 11).

FIGS. 8F and 8G depict the expression of HIPK2 in hMSCs at 72 hrs following treatment with either siRNA specific to HIPK2 or non-targeted control siRNA measured by qRT-PCR and Western blot. Fold change relative to siRNA control is shown (n=3, Mean±95% CI, ***p<0.001, t-test).

FIG. 8H depicts the gene expression of Wnt pathway genes AXIN2, LEF1, TCF7 and TCF4 in hMSCs at 72 hrs following treatment with either siRNA specific to HIPK2 or non-targeted control siRNA. Fold change relative to siRNA control is shown (n=3, Mean±95% CI, **p<0.01, ***p<0.001, t-test).

FIG. 9A depicts gene expression of SRSF4, 5, 6 in hMSCs at 72 hrs following treatment with either siRNA specific to SRSF4, SRSF5, SRSF6, combinations of siRNA, or non-targeted control siRNA measured by qRT-PCR. Fold change relative to siRNA control is shown (n=3, Mean±95% CI, *p<0.05, **p<0.01, ***p<0.001, one-way ANOVA).

FIG. 9B-9D depict gene expression of selected chondrocyte markers: COMP, SOX9, and RUNX1 (FIG. 9B), osteoblast marker RUNX2 (FIG. 9C), and CTNNB1 in hMSCs (FIG. 9D) at 72 hrs in cells from FIG. 9A. Fold change relative to siRNA control is shown (n=3, Mean±95% CI, *p<0.05, **p<0.01, ***p<0.001, one-way ANOVA).

FIG. 10A depicts expression of phospho- and total proteins in THP1 cells stimulated with LPS and treated with DMSO or compound 123 for 4 hrs or 20 hrs measured by Western blot, with β-actin as a loading control.

FIG. 10B depicts gene expression of RELA and RELB in THP1 cells stimulated with LPS and treated with DMSO or compound 123 for 4 hrs or 20 hrs measured by qRT-PCR. Fold change relative to unstimulated control is shown (n=3, Mean±95% CI, *p<0.05, **p<0.01, ***p<0.001, one-way ANOVA).

FIG. 11 depicts expression of phospho- and total proteins in THP1 cells stimulated with LPS and treated with DMSO or compound 123 for 10 mins, 30 mins, 1 hr, 2 hrs, 4 hrs, or 20 hrs measured by Western blot, with β-actin as a loading control. Compound 123 treatment showed no effects on phospho- or total NF-κB (p105/p50) at 10 mins, 30 mins, 1 hr or 2 hrs, but decreased both phospho- and total NF-κB at 4 hrs and 20 hrs, while phospho-STAT3 (Y705) was inhibited at 1 hr, 2 hrs, 4 hrs and 20 hrs, with no effects on total STAT3 at any timepoint.

FIG. 12 depicts gene expression of NF-κB pathway components in THP1 cells stimulated with LPS and treated with DMSO or compound 123 for 1 hr, 2 hrs, 4 hrs, or 20 hrs measured by qRT-PCR. Fold change relative to unstimulated control is shown (n=3, Mean±95% CI, *p<0.05, **p<0.01, ***p<0.001, one-way ANOVA). Compound 123 inhibited LPS-stimulated gene expression of NF-κB components (RELA, RELB), and inhibited the expression of NF-κB pathway genes (NFKB1, NFKB2, RELA, RELB and BCL3) without effects on NF-κB inhibitors (NFKBIA, NFKBIB, IKKβ, IKKγ).

FIG. 13 depicts expression of MMP-1, MMP-3, MMP-9 and MMP-13 in chondrocytes at 72 hrs following treatment with either siRNA specific to CLK2, CLK3 or DYRK1A and combinations of siRNA, or non-targeted control siRNA measured by qRT-PCR. Fold change relative to siRNA control is shown (n=3, Mean±95% CI, *p<0.05, **p<0.01, ***p<0.001, one-way ANOVA). Knockdowns of CLK2, CLK3 and DYRK1A either alone or in combination inhibited IL1-f induced expression of MMP-1, MMP-3, MMP-9 and MMP-13.

Like reference symbols in the various drawings indicate like elements.

### DETAILED DESCRIPTION

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “Wnt pathway activity” is an art-known term and generally refers to one or more direct Wnt/β-catenin activities in a mammalian cell and/or one or more indirect activities of Wnt/β-catenin (downstream activities resulting from Wnt/β-catenin activity) in a mammalian cell. Non-limiting examples of Wnt pathway activities include the level of expression of one or more Wnt-upregulated genes (e.g., one or more of any of the exemplary Wnt-upregulated genes described herein) in a mammalian cell, the level of β-catenin present in a nucleus of a mammalian cell, the level of expression of one or more of CLK1, CLK2, CLK3, CLK4, and β-catenin in a mammalian cell, detection of a gain-of-function mutation in a β-catenin gene, and detection of one or more of a loss-of-function mutation in one or more of a AXIN gene, a AXIN2 gene, a APC gene, a CTNNβ1 gene, a Tsc1 gene, a Tsc2 gene, a GSK3β gene, a SFRP3 gene, a Wnt7b gene, a WISP1 gene, a DKK1 gene, a DOTL1 gene, a FZDB gene, a LRP5 gene, and a LRP6 gene. Methods for detecting a level of each of these exemplary types of Wnt pathway activity are described herein. Additional examples of Wnt pathway activities are known in the art, as well as methods for detecting a level of the same.

As used herein, “gain-of-function mutation” means one or more nucleotide substitutions, deletions, and/or insertions in a gene that results in: an increase in the level of expression of the encoded protein as compared to the level of the expression by the corresponding wildtype gene, and/or the expression of a protein encoded by the gene that has one or more increased activities in a mammalian cell as compared to the version of the protein encoded by the corresponding wildtype gene.

As used herein, “loss-of-function mutation” means one or more nucleotide substitutions, deletions, and/or insertions in a gene that results in: a decrease in the level of expression of the encoded protein as compared to the level of the expression by the corresponding wildtype gene, and/or the expression of a protein encoded by the gene that has one or more decreased activities in a mammalian cell as compared to the version of the protein encoded by the corresponding wildtype gene.

As used herein, “Wnt-upregulated gene” means a gene that exhibits an increased level of transcription when the Wnt/β-catenin signaling pathway is active in a mammalian cell. Non-limiting examples of Wnt-upregulated genes are described herein. Additional examples of Wnt-upregulated genes are known in the art. Exemplary methods of detecting the level of expression of Wnt-upregulated genes are described herein. Additional methods of detecting the level of expression of Wnt-upregulated genes are known in the art. In some embodiments, Wnt-upregulated genes can be selected from one or more of the following: AES, AHR, APC, AXIN1, AXIN2, BAMBI, BCL9, BIRC5, BMP4, BTRC, CAMK2B, CCND1, CCND2, CCND3, CD44, CDH1, CDH11, CDKN2A, CEBPD, COL1A2, CREBBP, CSNK1A1, CSNK2A1, CTBP1, CTGF, CTNNB1, CUL1, CXCL12, CXCR4, CXXC4, DAB2, DIXDC1, DKK1, DKK2, DKK3, DKK4, DPP10, DVL1, DVL2, EFNB1, EGFR, EGR1, EP300, ERBB2, ETS2, FBXW11, FBXW4, FGF4, FGF7, FN1, FOSL1, FOXN1, FRAT1, FRZB, FZD1, FZD10, FZD2, FZD3, FZD4, FZD5, FZD6, FZD7, FZD8, FZD9, GDNF, GPC4, GSK3A, GSK3B, ID2, IGF1, IGF2, IL6, IRS1, JAG1, JUN, KLF5, KREMEN1, LEF1, LRP5, LRP6, MAPK10, MAPK8, MAPK9, MMP2, MMP3, MMP7, MMP9, MYC, MYLK, NANOG, NFATC1, NKD1, NLK, NRCAM, NRP1, PDGFRA, PITX2, PKN1, PLAUR, PLCB1, PLCB4, PORCN, POU5F1, PPAP2B, PPARD, PPP3CA, PPP3CB, PPP3CC, PPP3R1, PPP3R2, PRKACA, PRKACB, PRKACG, PRKCA, PRKCB, PRKCG, PRKX, PROM1, PTGS2, PYGO1, RAC1, RAC2, RAC3, RBX, RHOA, RUNX2, RUVBL, SERPINE1, SFRP1, SFRP2, SFRP4, SIX1, SKP1, SMAD2, SMAD3, SMAD4, SMO, SNAI1, SNAI2, SOST, SOX17, SOX2, SOX9, STAT3, TBL1XR1, TCF4, TCF7, TCF7L1, TGFB1, TGFB3, TIMP1, TLE1, TP53, TWIST1, TWIST2, VANGL1, VEGFA, WIF1, WISP1, WNT1, WNT10A, WNT10B, WNT11, WNT16, WNT2, WNT2B, WNT3, WNT4, WNT5A, WNT5B, WNT6, WNT7A, WNT7B, WNT8A, WNT8B, WNT9A, WNT9B, ZEB1, and ZEB2. In some embodiments, Wnt-upregulated genes can be selected from one or more of the following: CCND1, CXCL12, LRP5, AMP7, AMP9, LEF1, AXIN2, MYC, TCF7L2, TCF7, LRP6, DVL2, BIRC, ERRB2, MAPK8, PKN1, ABCB1, ADAM10, ALEX, ASCL2, BAMBI, BCL2L2, BIRC5, BMI, BMP4, CCND, CD44, CDKN2A, CDX, CEBPD, CLDN1, COX2, DNMT1, EDN1, EFNB1, ENC1, EPHB2, EPHB3, FGF18, FGFBP, FRA1, FSCN1, FZD7, FZD8, GAST, HEF1, HES1, ID2, ITF2, JAG1, JUN, L1CAM, LAMC2, LGR5, MENA, MET, MMP14, MYB, MYCBP, NOS2, NOTCH2, NRCAM, PLAU, PLAUR, PPARD, S100A4, S100A6, SGK1, SMC3, SOX9, SP5, SRSF3, SUZ12, TCF1, TIAM1, TIMP-1, TN-C, VEGF, WNT-5a, WNT-5b, WNT11, and YAP.

As used herein, “CLK inhibitor” refers to an agent (e.g., compound) that decreases the catalytic activity of one or more of CLK1, CLK2, CLK3, and CLK4 with an IC₅₀ of about 100 pM to about 10 μM (or any of the subranges of this range described herein) (e.g., determined using the exemplary in vitro assays for determining CLK1, CLK2, CLK3, and CLK4 activities described in the Examples).

As used herein, “a multi-isoform CLK inhibitor” refers to an agent (e.g., a compound that decreases the catalytic activity of two or more of CLK1, CLK2, CLK3, and CLK4 with an IC₅₀ of about 100 pM to about 10 μM (or any of the subranges of this range described herein) (e.g., determined using the exemplary in vitro assays for determining CLK1, CLK2, CLK3, and CLK4 activities described in the Examples).

As used herein, “DYRK inhibitor” refers to an agent (e.g., compound) that decreases the catalytic activity of one or more of DYRK1A, DYRK1B, DYRK2, DYRK3, and DYRK4 with an IC₅₀ of about 100 pM to about 10 μM (or any of the subranges of this range described herein) (e.g., determined using the exemplary in vitro assays for determining DYRK1A, DYRK1B, DYRK2, DYRK3, and DYRK4 activities described in the Examples).

As used herein, “a multi-isoform DYRK inhibitor” refers to an agent (e.g., a compound that decreases the catalytic activity of two or more of DYRK1A, DYRK1B, DYRK2, DYRK3, and DYRK4 with an IC₅₀ of about 100 pM to about 10 μM (or any of the subranges of this range described herein) (e.g., determined using the exemplary in vitro assays for determining DYRK1A, DYRK1B, DYRK2, DYRK3, and DYRK4 activities described in the Examples). As used herein, “altering mRNA splicing” means (i) changing the relative expression levels of two or more different isoforms of a protein in a mammalian cell that are encoded by the same gene, wherein the different isoforms of the protein result from mRNA splicing in the mammalian cell; and/or (ii) changing the level of activity, phosphorylation, and/or expression of one or more splicing factors in a mammalian cell. For example, altering mRNA splicing includes intron retention, exon skipping, premature stop codons, alternate 5′ splice site, alternate 3′ splice site, mutually exclusive exons, cassette exons, alternate promoters, and alternate polyadelynation sites.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched, or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched, or straight chain chemical group containing only carbon and hydrogen and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched, or straight chain chemical group containing only carbon and hydrogen and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “arylene” means a bivalent moiety obtained by removing two hydrogen atoms of an aryl ring, as defined above.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents as defined anywhere herein. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “heteroarylene” means a bivalent moiety obtained by removing two hydrogen atoms of a heteroaryl ring, as defined above.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched, alkyl, alkenyl or alkynyl substituted with one or more chloro, bromo, fluoro, and/or iodo atom(s). In some embodiments, a haloalkyl is a fluoroalkyls, wherein one or more of the hydrogen atoms have been substituted by fluoro. In some embodiments, haloalkyls are of 1 to about 3 carbons in length (e.g., 1 to about 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused and/or bridged rings. Heterocyclyls may be substituted or unsubstituted with one or more substituents as defined anywhere herein. In some embodiments, heterocycles have 3-11 members. In six membered monocyclic heterocycles, the heteroatom(s) are selected from one to three of O, N or S, and wherein when the heterocycle is five membered, it can have one or two heteroatoms selected from O, N, or S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents as defined anywhere herein. In some embodiments, heterocycles have 3-7 members. In six membered monocyclic heterocycles, the heteroatom(s) are selected from one to three of O, N or S, and wherein when the heterocycle is five membered, it can have one or two heteroatoms selected from O, N, or S. Examples of heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “bicyclic heterocyclyl” means a nonaromatic bicyclic ring system comprising at least one heteroatom in the ring system backbone. Bicyclic heterocyclyls may be substituted or unsubstituted with one or more substituents as defined anywhere herein, and may include multiple fused and/or bridged rings. In some embodiments, bicyclic heterocycles have 4-11 members with the heteroatom(s) being selected from one to five of O, N or S. Examples of bicyclic heterocyclyls include 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, and the like.

As used herein, “spirocyclic heterocyclyl” means a nonaromatic bicyclic ring system comprising at least one heteroatom in the ring system backbone and with the rings connected through just one atom (the “spiroatom”). Spirocyclic heterocyclyls may be substituted or unsubstituted with one or more substituents as defined anywhere herein. In some embodiments, spirocyclic heterocycles have 5-11 members with the heteroatom(s) being selected from one to five of O, N or S. Examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 2,5-diazaspiro[3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R″; —NRR′; —C(O)NRR′; —C(NR)NR′ R″; —C(NR′)R″; a cyano; a nitro; an azido; —SH; —S—R; —OSO₂(OR); a sulfonate [such as —SO₂(OH) and —SO₂(OR)]; —SO₂NR′ R″; and —SO₂R; in which each occurrence of R, R′ and R″ are independently selected from H; —(C₁₋₉ alkyl); C₆₋₁₀ aryl optionally substituted with from 1-3R′″; 5-10 membered heteroaryl having from 1-4 heteroatoms independently selected from N, O, and S and optionally substituted with from 1-3 R′″; C₃₋₇ carbocyclyl optionally substituted with from 1-3 R′″; and 3-8 membered heterocyclyl having from 1-4 heteroatoms independently selected from N, O, and S and optionally substituted with from 1-3 R′″; wherein each R′″ is independently selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically favored, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formulas (I)-(VIII) wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

The term “polymorph,” as used herein, refers to crystals of the same molecule having different physical properties as a result of the order of the molecules in the crystal lattice. Polymorphs of a single compound have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bio-availability), solubility, melting point, chemical stability, physical stability, powder flowability, water sorption, compaction, and particle morphology. Differences in stability can result from changes in chemical reactivity (e.g. differential oxidation, such that a dosage form discolors more rapidly when comprised of one polymorph than when comprised of another polymorph) or mechanical changes (e.g., crystal changes on storage as a kinetically favored polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph” does not include amorphous forms of the compound. As used herein, “amorphous” refers to a noncrystalline form of a compound which may be a solid state form of the compound or a solubilized form of the compound. For example, “amorphous” refers to a compound without a regularly repeating arrangement of molecules or external face planes.

The term “anhydrous,” as used herein, refers to a crystal form of the compound of Formulas (I)-(VIII), or a pharmaceutically acceptable salt thereof, that has 1% or less by weight water. For example, 0.5% or less, 0.25% or less, or 0.1% or less by weight water.

The term “solvate” as used herein refers to a crystalline form of a compound of Formulas (I)-(VIII), or a pharmaceutically acceptable salt thereof, such as a polymorph form of the compound, where the crystal lattice comprises one or more solvents of crystallization.

The term “non-stoichiometric hydrate” refers to a crystalline form of a compound of Formulas (I)-(VIII) or a pharmaceutically acceptable salt thereof, that comprises water, but wherein variations in the water content do not cause significant changes to the crystal structure. In some embodiments, a non-stoichiometric hydrate can refer to a crystalline form of a compound of Formulas (I)-(VIII) that has channels or networks throughout the crystal structure into which water molecules can diffuse. During drying of non-stoichiometric hydrates, a considerable proportion of water can be removed without significantly disturbing the crystal network, and the crystals can subsequently rehydrate to give the initial non-stoichiometric hydrated crystalline form. Unlike stoichiometric hydrates, the dehydration and rehydration of non-stoichiometric hydrates is not accompanied by a phase transition, and thus all hydration states of a non-stoichiometric hydrate represent the same crystal form. In some embodiments, a non-stoichiometric hydrate can have up to about 20% by weight water, such as, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or greater than 1% water by weight. In some embodiments, a non-stoichiometric hydrate can have between 1% and about 20% by weight water, such as between 1% and about 5%, 1% and about 10%, 1% and about 15%, about 2% and about 5%, about 2% and about 10%, about 2% and about 15%, about 2% and about 20%, about 5% and about 10%, about 5% and about 15%, about 5% and about 20%, about 10% and about 15%, about 10% and about 20%, or about 15% and about 20% by weight water.

In some embodiments the % water by weight in a crystal form, such as a non-stoichiometric hydrate, is determined by the Karl Fischer titration method. In some embodiments, the crystal form is dried prior to Karl Fischer titration. In some embodiments, one or more of the first compound, the second compound, and the dual DYRK1A/CLK2 and/or CLK3 inhibitor can each independently be substantially present as a non-stoichiometric hydrate.

“Purity,” when used in reference to a composition including a polymorph of a compound of Formulas (I)-(VIII), or a pharmaceutically acceptable salt thereof, refers to the percentage of one specific polymorph form relative to another polymorph form or an amorphous form of a compound of Formulas (I)-(VIII), or a pharmaceutically acceptable salt thereof, in the referenced composition. For example, a composition comprising a polymorph of Formulas (I)-(VIII) having a purity of 90% would comprise 90 weight parts Form 1 and 10 weight parts of other polymorph and/or amorphous forms of the corresponding compound of Formulas (I)-(VIII).

As used herein, a compound or composition is “substantially free of” one or more other components if the compound or composition contains no significant amount of such other components. Such components can include starting materials, residual solvents, or any other impurities that can result from the preparation of and/or isolation of the compounds and compositions provided herein. In some embodiments, a polymorph form provided herein is substantially free of other polymorph forms. In some embodiments, a particular polymorph of the compound of Formulas (I)-(VIII), or a pharmaceutically acceptable salt thereof, is “substantially free” of other polymorphs if the particular polymorph constitutes at least about 95% by weight of the compound of Formulas (I)-(VIII), or a pharmaceutically acceptable salt thereof, present. In some embodiments, a particular polymorph of the compound of Formulas (I)-(VIII), or a pharmaceutically acceptable salt thereof, is “substantially free” of other polymorphs if the particular polymorph constitutes at least about 97%, about 98%, about 99%, or about 99.5% by weight of the compound of Formulas (I)-(VIII), or a pharmaceutically acceptable salt thereof, present. In certain embodiments, a particular polymorph of the compound of Formulas (I)-(VIII), or a pharmaceutically acceptable salt thereof, is “substantially free” of water if the amount of water constitutes no more than about 2%, about 1%, or about 0.5% by weight of the polymorph.

As used herein, a compound is “substantially present” as a given polymorph if at least about 50% by weight of the compound is in the form of that polymorph. In some embodiments, at least about 60% by weight of the compound is in the form of that polymorph. In some embodiments, at least about 70% by weight of the compound is in the form of that polymorph. In some embodiments, at least about 80% by weight of the compound is in the form of that polymorph. In some embodiments, at least about 90% by weight of the compound is in the form of that polymorph. In some embodiments, at least about 95% by weight of the compound is in the form of that polymorph. In some embodiments, at least about 96% by weight of the compound is in the form of that polymorph. In some embodiments, at least about 97% by weight of the compound is in the form of that polymorph. In some embodiments, at least about 98% by weight of the compound is in the form of that polymorph. In some embodiments, at least about 99% by weight of the compound is in the form of that polymorph. In some embodiments, at least about 99.5% by weight of the compound is in the form of that polymorph.

“Room temperature” or “RT” refers to the ambient temperature of a typical laboratory, which is typically around 25° C.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” or “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Brunton et al. (Eds.) (2017); Goodman and Gilman's: The Pharmacological Basis of Therapeutics, 13th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formulas (I)-(VIII), or a pharmaceutically acceptable salt or solvate thereof, in combination with one or more other agents that are effective to treat the diseases and/or conditions described herein. When referring to combinations of compounds, the combination may be “therapeutically effective” even when one or more of the compounds in the combination is administered at a dose that would be sub-therapeutic when the compound is administered alone. Indeed, the combination of compounds, or pharmaceutically acceptable salts or solvates of the foregoing, can be an additive combination, or can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon other art-recognized factors, for example, the patient's height, weight, sex, age and medical history.

The term “combination therapy” as used herein refers to a dosing regimen of two different therapeutically active agents (i.e., the components or combination partners of the combination) (e.g., a first compound and a second compound) during a period of time, wherein the therapeutically active agents are administered together or separately in a manner prescribed by a medical care provider or according to a regulatory agency (e.g., the U.S. Food and Drug Administration, the European Medicines Agency, etc.). In some embodiments, a combination therapy consists essentially of a combination of a first compound (e.g., a compound of Formulas (I)-(VIII)), or a pharmaceutically acceptable salt or solvate thereof, and a second compound (e.g., a compound of Formulas (I)-(VIII)), or a pharmaceutically acceptable salt or solvate thereof.

As can be appreciated in the art, a combination therapy can be administered to a patient for a period of time. In some embodiments, the period of time occurs following the administration of a different therapeutic treatment/agent or a different combination of therapeutic treatments/agents to the subject, as described herein (e.g., non-steroidal anti-inflammatory therapy, physical therapy, etc.). In some embodiments, the period of time occurs before the administration of a different therapeutic treatment/agent or a different combination of therapeutic treatments/agents to the subject, as described herein. In some embodiments, administration of the first compound, or a pharmaceutically acceptable salt or solvate thereof, and administration of the second compound, or a pharmaceutically acceptable salt or solvate thereof, occurs at substantially the same time. In other embodiments, administration of the first compound, or a pharmaceutically acceptable salt or solvate thereof, and administration of the second compound, or a pharmaceutically acceptable salt or solvate thereof, occurs sequentially, in either order (e.g., the first compound, or a pharmaceutically acceptable salt or solvate thereof, may be administered prior to, or subsequent to, the second compound, or a pharmaceutically acceptable salt or solvate thereof). A therapeutic effect refers to the treatment of a disease or condition, as described herein.

Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating one or more existing symptoms, ameliorating the underlying metabolic causes of symptoms, postponing or preventing the further development of a disorder, and/or reducing the severity of one or more symptoms that will or are expected to develop. “Treat,” “treatment,” and “treating,” do not necessarily result in completely curing an underlying disease or condition.

The phrase “an elevated” or “an increased level” as used herein can be an increase of at least 1% (e.g., at least 2%, at least 4%, at least 6%, at least 8%, at least 10%, at least 12%, at least 14%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 120%, at least 130%, at least 140%, at least 150%, at least 160%, at least 170%, at least 180%, at least 190%, at least 200%, at least 250%, at least 300%, at least 350%, at least 400%, at least 450%, at least 500%, between 1% and 500%, between 1% and 450%, between 1% and 400%, between 1% and 350%, between 1% and 300%, between 1% and 250%, between 1% and 200%, between 1% and 180%, between 1% and 160%, between 1% and 140%, between 1% and 120%, between 1% and 100%, between 1% and 95%, between 1% and 90%, between 1% and 85%, between 1% and 80%, between 1% and 75%, between 1% and 70%, between 1% and 65%, between 1% and 60%, between 1% and 55%, between 1% and 50%, between 1% and 45%, between 1% and 40%, between 1% and 35%, between 1% and 30%, between 1% and 25%, between 1% and 20%, between 1% and 15%, between 1% and 10%, between 1% and 5%, between 5% and 500%, between 5% and 450%, between 5% and 400%, between 5% and 350%, between 5% and 300%, between 5% and 250%, between 5% and 200%, between 5% and 180%, between 5% and 160%, between 5% and 140%, between 5% and 120%, between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 500%, between 10% and 450%, between 10% and 400%, between 10% and 350%, between 10% and 300%, between 10% and 250%, between 10% and 200%, between 10% and 180%, between 10% and 160%, between 10% and 140%, between 10% and 120%, between 10% and 100%, between 10% and 95%, between 10% and 90%, between 10% and 85%, between 10% and 80%, between 10% and 75%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 20% and 500%, between 20% and 450%, between 20% and 400%, between 20% and 350%, between 20% and 300%, between 20% and 250%, between 20% and 200%, between 20% and 180%, between 20% and 160%, between 20% and 140%, between 20% and 120%, between 20% and 100%, between 20% and 95%, between 20% and 90%, between 20% and 85%, between 20% and 80%, between 20% and 75%, between 20% and 70%, between 20% and 65%, between 20% and 60%, between 20% and 55%, between 20% and 50%, between 20% and 45%, between 20% and 40%, between 20% and 35%, between 20% and 30%, between 20% and 25%, between 30% and 500%, between 30% and 450%, between 30% and 400%, between 30% and 350%, between 30% and 300%, between 30% and 250%, between 30% and 200%, between 30% and 180%, between 30% and 160%, between 30% and 140%, between 30% and 120%, between 30% and 100%, between 30% and 95%, between 30% and 90%, between 30% and 85%, between 30% and 80%, between 30% and 75%, between 30% and 70%, between 30% and 65%, between 30% and 60%, between 30% and 55%, between 30% and 50%, between 30% and 45%, between 30% and 40%, between 30% and 35%, between 40% and 500%, between 40% and 450%, between 40% and 400%, between 40% and 350%, between 40% and 300%, between 40% and 250%, between 40% and 200%, between 40% and 180%, between 40% and 160%, between 40% and 140%, between 40% and 120%, between 40% and 100%, between 40% and 95%, between 40% and 90%, between 40% and 85%, between 40% and 80%, between 40% and 75%, between 40% and 70%, between 40% and 65%, between 40% and 60%, between 40% and 55%, between 40% and 50%, between 40% and 45%, between 50% and 500%, between 50% and 450%, between 50% and 400%, between 50% and 350%, between 50% and 300%, between 50% and 250%, between 50% and 200%, between 50% and 180%, between 50% and 160%, between 50% and 140%, between 50% and 120%, between 50% and 100%, between 50% and 95%, between 50% and 90%, between 50% and 85%, between 50% and 80%, between 50% and 75%, between 50% and 70%, between 50% and 65%, between 50% and 60%, between 50% and 55%, between 60% and 500%, between 60% and 450%, between 60% and 400%, between 60% and 350%, between 60% and 300%, between 60% and 250%, between 60% and 200%, between 60% and 180%, between 60% and 160%, between 60% and 140%, between 60% and 120%, between 60% and 100%, between 60% and 95%, between 60% and 90%, between 60% and 85%, between 60% and 80%, between 60% and 75%, between 60% and 70%, between 60% and 65%, between 70% and 500%, between 70% and 450%, between 70% and 400%, between 70% and 350%, between 70% and 300%, between 70% and 250%, between 70% and 200%, between 70% and 180%, between 70% and 160%, between 70% and 140%, between 70% and 120%, between 70% and 100%, between 70% and 95%, between 70% and 90%, between 70% and 85%, between 70% and 80%, between 70% and 75%, between 80% and 500%, between 80% and 450%, between 80% and 400%, between 80% and 350%, between 80% and 300%, between 80% and 250%, between 80% and 200%, between 80% and 180%, between 80% and 160%, between 80% and 140%, between 80% and 120%, between 80% and 100%, between 80% and 95%, between 80% and 90%, between 80% and 85%, between 90% and 500%, between 90% and 450%, between 90% and 400%, between 90% and 350%, between 90% and 300%, between 90% and 250%, between 90% and 200%, between 90% and 180%, between 90% and 160%, between 90% and 140%, between 90% and 120%, between 90% and 100%, between 90% and 95%, between 100% and 500%, between 100% and 450%, between 100% and 400%, between 100% and 350%, between 100% and 300%, between 100% and 250%, between 100% and 200%, between 100% and 180%, between 100% and 160%, between 100% and 140%, between 100% and 120%, between 120% and 500%, between 120% and 450%, between 120% and 400%, between 120% and 350%, between 120% and 300%, between 120% and 250%, between 120% and 200%, between 120% and 180%, between 120% and 160%, between 120% and 140%, between 140% and 500%, between 140% and 450%, between 140% and 400%, between 140% and 350%, between 140% and 300%, between 140% and 250%, between 140% and 200%, between 140% and 180%, between 140% and 160%, between 160% and 500%, between 160% and 450%, between 160% and 400%, between 160% and 350%, between 160% and 300%, between 160% and 250%, between 160% and 200%, between 160% and 180%, between 180% and 500%, between 180% and 450%, between 180% and 400%, between 180% and 350%, between 180% and 300%, between 180% and 250%, between 180% and 200%, between 200% and 500%, between 200% and 450%, between 200% and 400%, between 200% and 350%, between 200% and 300%, between 200% and 250%, between 250% and 500%, between 250% and 450%, between 250% and 400%, between 250% and 350%, between 250% and 300%, between 300% and 500%, between 300% and 450%, between 300% and 400%, between 300% and 350%, between 350% and 500%, between 350% and 450%, between 350% and 400%, between 400% and 500%, between 400% and 450%, or about 450% to about 500%), e.g., as compared to a reference level (e.g., any of the exemplary reference levels described herein).

As used herein, a “first time point” can, e.g., refer to a designated time point, which can, e.g., be used to refer to chronologically later time points (e.g., a second time point). In some examples, a subject may not have yet received a treatment at a first time point (e.g., may not have yet received a dose of a dual CLK/DYRK inhibitor, or a combination of a CLK inhibitor and a DYRK inhibitor (e.g., any of the CLK or DYRK inhibitors described herein) at a first time point). In some examples, a subject may have already received a treatment that does not include a dual CLK/DYRK inhibitor, or a combination of a CLK inhibitor and a DYRK inhibitor at the first time point. In some examples, the previous treatment that does not include a dual CLK/DYRK inhibitor, or a combination of a CLK inhibitor and a DYRK inhibitor was identified as being ineffective prior to the first time point. In some examples, a subject has previously been identified or diagnosed as having a cartilage disorders (e.g., any of the types of cancer described herein or known in the art) at the first time point. In some examples, a subject has previously been suspected of having a cartilage disorders (e.g., any of the types of cartilage disorders described herein or known in the art) at the first time point. In other examples, a first time point can be a time point when a subject has developed at least one (e.g., 1, 2, 3, 4, 5, 6, 7, 8, 9, or 10) symptom(s) associated with a cartilage disorders and has not yet received any treatment for cartilage disorders.

As used herein, a “second time point” refers to a time point that occurs chronologically after a first designated time point. In some examples, a subject (e.g., any of the subjects described herein) can receive or has received at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) doses of a treatment (e.g., a dual CLK/DYRK inhibitor or a combination of a CLK inhibitor and a DYRK inhibitor (e.g., any of the CLK or DYRK inhibitors described herein)) between the first and the second time points. In some examples, the time difference between a first and a second time point can be, e.g., 1 day to about 12 months, 1 day to about 11 months, 1 day to about 10 months, 1 day to about 9 months, 1 day to about 8 months, 1 day to about 7 months, 1 day to about 6 months, 1 day to about 22 weeks, 1 day to about 20 weeks, 1 day to about 18 weeks, 1 day to about 16 weeks, 1 day to about 14 weeks, 1 day to about 12 weeks, 1 day to about 10 weeks, 1 day to about 8 weeks, 1 day to about 6 weeks, 1 day to about 4 weeks, 1 day to about 3 weeks, 1 day to about 2 weeks, 1 day to about 1 week, about 2 days to about 12 months, about 2 days to about 11 months, about 2 days to about 10 months, about 2 days to about 9 months, about 2 days to about 8 months, about 2 days to about 7 months, about 2 days to about 6 months, about 2 days to about 22 weeks, about 2 days to about 20 weeks, about 2 days to about 18 weeks, about 2 days to about 16 weeks, about 2 days to about 14 weeks, about 2 days to about 12 weeks, about 2 days to about 10 weeks, about 2 days to about 8 weeks, about 2 days to about 6 weeks, about 2 days to about 4 weeks, about 2 days to about 3 weeks, about 2 days to about 2 weeks, about 2 days to about 1 week, about 4 days to about 12 months, about 4 days to about 11 months, about 4 days to about 10 months, about 4 days to about 9 months, about 4 days to about 8 months, about 4 days to about 7 months, about 4 days to about 6 months, about 4 days to about 22 weeks, about 4 days to about 20 weeks, about 4 days to about 18 weeks, about 4 days to about 16 weeks, about 4 days to about 14 weeks, about 4 days to about 12 weeks, about 4 days to about 10 weeks, about 4 days to about 8 weeks, about 4 days to about 6 weeks, about 4 days to about 4 weeks, about 4 days to about 3 weeks, about 4 days to about 2 weeks, about 4 days to about 1 week, about 1 week to about 12 months, about 1 week to about 11 months, about 1 week to about 10 months, about 1 week to about 9 months, about 1 week to about 8 months, about 1 week to about 7 months, about 1 week to about 6 months, about 1 week to about 22 weeks, about 1 week to about 20 weeks, about 1 week to about 18 weeks, about 1 week to about 16 weeks, about 1 week to about 14 weeks, about 1 week to about 12 weeks, about 1 week to about 10 weeks, about 1 week to about 8 weeks, about 1 week to about 6 weeks, about 1 week to about 4 weeks, about 1 week to about 3 weeks, about 1 week to about 2 weeks, about 2 weeks to about 12 months, about 2 weeks to about 11 months, about 2 weeks to about 10 months, about 2 weeks to about 9 months, about 2 weeks to about 8 months, about 2 weeks to about 7 months, about 2 weeks to about 6 months, about 2 weeks to about 22 weeks, about 2 weeks to about 20 weeks, about 2 weeks to about 18 weeks, about 2 weeks to about 16 weeks, about 2 weeks to about 14 weeks, about 2 weeks to about 12 weeks, about 2 weeks to about 10 weeks, about 2 weeks to about 8 weeks, about 2 weeks to about 6 weeks, about 2 weeks to about 4 weeks, about 2 weeks to about 3 weeks, about 3 weeks to about 12 months, about 3 weeks to about 11 months, about 3 weeks to about 10 months, about 3 weeks to about 9 months, about 3 weeks to about 8 months, about 3 weeks to about 7 months, about 3 weeks to about 6 months, about 3 weeks to about 22 weeks, about 3 weeks to about 20 weeks, about 3 weeks to about 18 weeks, about 3 weeks to about 16 weeks, about 3 weeks to about 14 weeks, about 3 weeks to about 12 weeks, about 3 weeks to about 10 weeks, about 3 weeks to about 8 weeks, about 3 weeks to about 6 weeks, about 3 weeks to about 4 weeks, about 4 weeks to about 12 months, about 4 weeks to about 11 months, about 4 weeks to about 10 months, about 4 weeks to about 9 months, about 4 weeks to about 8 months, about 4 weeks to about 7 months, about 4 weeks to about 6 months, about 4 weeks to about 22 weeks, about 4 weeks to about 20 weeks, about 4 weeks to about 18 weeks, about 4 weeks to about 16 weeks, about 4 weeks to about 14 weeks, about 4 weeks to about 12 weeks, about 4 weeks to about 10 weeks, about 4 weeks to about 8 weeks, about 4 weeks to about 6 weeks, about 6 weeks to about 12 months, about 6 weeks to about 11 months, about 6 weeks to about 10 months, about 6 weeks to about 9 months, about 6 weeks to about 8 months, about 6 weeks to about 7 months, about 6 weeks to about 6 months, about 6 weeks to about 22 weeks, about 6 weeks to about 20 weeks, about 6 weeks to about 18 weeks, about 6 weeks to about 16 weeks, about 6 weeks to about 14 weeks, about 6 weeks to about 12 weeks, about 6 weeks to about 10 weeks, about 6 weeks to about 8 weeks, about 8 weeks to about 12 months, about 8 weeks to about 11 months, about 8 weeks to about 10 months, about 8 weeks to about 9 months, about 8 weeks to about 8 months, about 8 weeks to about 7 months, about 8 weeks to about 6 months, about 8 weeks to about 22 weeks, about 8 weeks to about 20 weeks, about 8 weeks to about 18 weeks, about 8 weeks to about 16 weeks, about 8 weeks to about 14 weeks, about 8 weeks to about 12 weeks, about 8 weeks to about 10 weeks, about 10 weeks to about 12 months, about 10 weeks to about 11 months, about 10 weeks to about 10 months, about 10 weeks to about 9 months, about 10 weeks to about 8 months, about 10 weeks to about 7 months, about 10 weeks to about 6 months, about 10 weeks to about 22 weeks, about 10 weeks to about 20 weeks, about 10 weeks to about 18 weeks, about 10 weeks to about 16 weeks, about 10 weeks to about 14 weeks, about 10 weeks to about 12 weeks, about 12 weeks to about 12 months, about 12 weeks to about 11 months, about 12 weeks to about 10 months, about 12 weeks to about 9 months, about 12 weeks to about 8 months, about 12 weeks to about 7 months, about 12 weeks to about 6 months, about 12 weeks to about 22 weeks, about 12 weeks to about 20 weeks, about 12 weeks to about 18 weeks, about 12 weeks to about 16 weeks, about 12 weeks to about 14 weeks, about 14 weeks to about 12 months, about 14 weeks to about 11 months, about 14 weeks to about 10 months, about 14 weeks to about 9 months, about 14 weeks to about 8 months, about 14 weeks to about 7 months, about 14 weeks to about 6 months, about 14 weeks to about 22 weeks, about 14 weeks to about 20 weeks, about 14 weeks to about 18 weeks, about 14 weeks to about 16 weeks, about 16 weeks to about 12 months, about 16 weeks to about 11 months, about 16 weeks to about 10 months, about 16 weeks to about 9 months, about 16 weeks to about 8 months, about 16 weeks to about 7 months, about 16 weeks to about 6 months, about 16 weeks to about 22 weeks, about 16 weeks to about 20 weeks, about 16 weeks to about 18 weeks, about 18 weeks to about 12 months, about 18 weeks to about 11 months, about 18 weeks to about 10 months, about 18 weeks to about 9 months, about 18 weeks to about 8 months, about 18 weeks to about 7 months, about 18 weeks to about 6 months, about 18 weeks to about 22 weeks, about 18 weeks to about 20 weeks, about 20 weeks to about 12 months, about 20 weeks to about 11 months, about 20 weeks to about 10 months, about 20 weeks to about 9 months, about 20 weeks to about 8 months, about 20 weeks to about 7 months, about 20 weeks to about 6 months, about 20 weeks to about 22 weeks, about 22 weeks to about 12 months, about 22 weeks to about 11 months, about 22 weeks to about 10 months, about 22 weeks to about 9 months, about 22 weeks to about 8 months, about 22 weeks to about 7 months, about 22 weeks to about 6 months, about 24 weeks to about 12 months, about 24 weeks to about 11 months, about 24 weeks to about 10 months, about 24 weeks to about 9 months, about 24 weeks to about 8 months, about 24 weeks to about 7 months, about 7 months to about 12 months, about 7 months to about 11 months, about 7 months to about 10 months, about 7 months to about 9 months, about 7 months to about 8 months, about 8 months to about 12 months, about 8 months to about 11 months, about 8 months to about 10 months, about 8 months to about 9 months, about 9 months to about 12 months, about 9 months to about 11 months, about 9 months to about 10 months, about 10 months to about 12 months, about 10 months to about 11 months, or about 11 months to about 12 months.

### Methods of Treatment

The present disclosure is based on the surprising discovery that DYRK and CLK signaling is important in chondrogenesis. Specifically, the present application describes how inhibition of DYRK (e.g., DYKR1A) and CLK (e.g., CLK2 and/or CLK3) signaling results in unexpectedly superior treatment of a variety of disorders, for example, osteoarthritis and degenerative disc disease (DDD), amongst many others. These surprising and unexpected effects can be accomplished through administration of a CLK inhibitor and a DYRK inhibitor or a single agent capable of inhibiting both targets. While such treatment may be generally beneficial to patients suffering from, or at risk of suffering from, a variety of disorders, some patients may also be specifically selected for such treatment. For example, patients having an elevated level of Wnt pathway activity as compared to a reference level and/or possessing certain biomarkers for one or more cartilage-related disorders.

Some embodiments provide methods of treating osteoarthritis in a subject, methods of selecting a treatment for a subject, methods of selecting a subject for treatment, that each include identifying a subject having an elevated level of Wnt pathway activity as compared to a reference level. Also provided herein are methods of determining the efficacy of a DYRK inhibitor and a CLK inhibitor in a subject that include detecting a level of Wnt/β-catenin signaling activity in a sample obtained from the subject. Also provided are methods of decreasing the activity of DYRK and CLK that include the use of any of the inhibitors or pharmaceutically acceptable salts or solvates thereof described herein. Also provided herein are methods of treating osteoarthritis using a DYRK inhibitor and a CLK inhibitor, methods of selecting a subject for treatment with a DYRK inhibitor and a CLK inhibitor, methods of increasing chondrogenesis using a DYRK inhibitor and a CLK inhibitor, methods of modifying the progression of osteoarthritis using a DYRK inhibitor and a CLK inhibitor, that each include the use of a DYRK inhibitor and a CLK inhibitor, that include a step of identifying a subject having elevated Wnt pathway activity. In some embodiments, the DYRK inhibitor and the CLK inhibitor are separate compounds (e.g., a first compound and a second compound). In some embodiments, the DRYK inhibitor and the CLK inhibitor are the same compound (i.e., a dual CLK/DYRK inhibitor).

### Exemplary Conditions

Non-limiting examples of diseases which can be treated with a dual CLK/DYRK inhibitor or a combination of a CLK inhibitor and DYRK inhibitor, or a pharmaceutically acceptable salt or solvate of any of the foregoing, are axial spondyloarthritis (including ankylosing spondylitis), costochondritis, degenerative disc disease, degenerative spondylolisthesis (also called degenerative anterolisthesis), elbow dysplasia, gout, juvenile idiopathic arthritis, osteoarthritis, osteochondritis dissecans, Panner disease, reactive arthritis, relapsing polychondritis, rheumatoid arthritis (RA), sacroiliac joint dysfunction, septic arthritis, Still's disease, Tietze syndrome (also called chondropathia tuberosa or costochondral junction syndrome).

Non-limiting examples of diseases where joint pain, join inflammation, and cartilage damage can also be a symptom and can be treated with a dual CLK/DYRK inhibitor or a combination of a CLK inhibitor and DYRK inhibitor, or a pharmaceutically acceptable salt or solvate of any of the foregoing, are psoriasis (psoriatic arthritis), reactive arthritis, Ehlers-Danlos syndrome, haemochromatosis, hepatitis, Lyme disease, Sjogren's disease, Hashimoto's thyroiditis, Celiac disease, non-celiac gluten sensitivity, inflammatory bowel disease (including Crohn's disease and ulcerative colitis), Henoch-Schonlein purpura, hyperimmunoglobulinemia D with recurrent fever, sarcoidosis, Whipple's disease, TNF receptor associated periodic syndrome, granulomatosis with polyangiitis (and many other vasculitis syndromes), familial Mediterranean fever, and systemic lupus erythematosus.

Cartilage that can be treated with a dual CLK/DYRK inhibitor or a combination of a CLK inhibitor and DYRK inhibitor, or a pharmaceutically acceptable salt or solvate of any of the foregoing, can be located anywhere in the body, i.e. joints between bones (e.g. the elbows, knees and ankles), pubic symphysis (which is the position at which the hip bones join at the front of the body), ends of the ribs, menisci (cartilage pads of the knee joint), intervertebral discs, auditory (eustachian) tubes, auricle (external ears), bronchi, bronchial tubes, costal cartilages, larynx (voice-box), nose, trachea, epiglottis (the lid on the top of the larynx). The cartilage can be of any type, i.e. hyaline cartilage, elastic cartilage, fibrocartilage, or articular cartilage.

As used herein, “chondrogenesis” refers to the process by which cartilage is formed from condensed mesenchyme tissue, which differentiates into chondrocytes and begins secreting the molecules that form the extracellular matrix. Additionally, chondrogenesis is sometimes referred to as chondrification. Specifically, chondrogenesis occurs as a result of condensation of mesenchymal cells, which express collagens I, III, and V and chondroprogenitor cell differentiation with expression of cartilage-specific collagens II, IX, and XI. Additional molecular players involved in chondrogenesis include, but are not limited to aggrecan (Agc1), Sonic Hedgehog (Shh), Patched-1 and 2 (Ptch1,2), Smoothened (Smo), Gli, Sox5, Sox6, Sox9, Nkx3-2, CREB, NFAT4, FGFs, HIF-1α, TGF-β, BMP, PKA, PKC, PP2A, PP2B, ERK1/2, p38, JNK, N-cam, N-cadherin, Integrin α5β1, and Wnt. It is the interplay between stimulatory and inhibitory factors that controls the rate and progression of chondrogenesis. In adults, chondrocytes are the single cellular component of articular cartilage. Articular cartilage has a limited capacity for healing and repair, due to the low turnover equilibrium of chondrocytes.

In some embodiments, the methods described herein induce chondrogenesis by administering a first compound, wherein the first compound is a CLK2 and/or CLK3 inhibitor or a pharmaceutically acceptable salt or solvate thereof, and a second compound, wherein the second compound is a DYRK1A inhibitor, or a pharmaceutically acceptable salt or solvate thereof. In some embodiments, the methods described herein induce chondrogenesis by administering a therapeutically effective amount of a single compound, or a pharmaceutically acceptable salt or solvate thereof, wherein the single compound is a dual DYRA1A/CLK2 and/or CLK3 inhibitor, which inhibits both DYRK1A and CLK2 and/or CLK3. In some embodiments, the methods activate chondrogenic mesenchymal cells to become differentiated chondrocytes. In some embodiments, administration of one or more compounds discussed herein generates differentiated chondrocytes. In some embodiments, administration of one or more compounds discussed herein produces chondrogenic nodules or differentiated chondrocytes. In some embodiments, administration of one or more compounds discussed herein promotes increased cartilage growth.

In some embodiments, the CLK inhibitor, the DYRK inhibitor, and the dual DYRK/CLK inhibitor, are each independently selected from compounds of Formulas (I)-(VIII), or a pharmaceutically acceptable salt or solvate thereof.

### Spondylosis

Spondylosis is the degeneration of the spinal column from any cause. In the more narrow sense it refers to spinal osteoarthritis, the age-related wear and tear of the spinal column, which is the most common cause of spondylosis. The degenerative process in osteoarthritis chiefly affects the vertebral bodies, the neural foramina and the facet joints (facet syndrome). If severe, it may cause pressure on the spinal cord or nerve roots with subsequent sensory or motor disturbances, such as pain, paresthesia, imbalance, and muscle weakness in the limbs.

In some embodiments, the methods disclosed herein can be used to treat spondylosis. In some embodiments, the methods disclosed herein can be used to prevent further cartilage breakdown after a spondylosis diagnosis. In some embodiments, the methods disclosed herein can be used to induce chondrocyte differentiation a spondylosis diagnosis. Additional methods disclosed herein can be used to facilitate recovery after a spondylosis diagnosis. In some embodiments, provided herein are methods of modifying the progression of spondylosis.

Provided herein are methods for treating spondylosis in a subject comprising administering to the subject a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof. Additionally, methods for treating degenerative disc disease are provided comprising administering to the subject a single compound or a pharmaceutically acceptable salt or solvate thereof, wherein the single compound is a dual DYRK/CLK inhibitor, which inhibits CLK and DYRK. In some embodiments, the methods for treating spondylosis in a subject comprise administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. Additionally, methods for treating spondylosis are provided comprising administering to the subject a single compound or a pharmaceutically acceptable salt or solvate thereof, wherein the single compound is a dual DYRK1A/CLK2 and/or CLK3 inhibitor, which inhibits DYRK1A and CLK2 and/or CLK3.

### Degenerative Disc Disease

Degenerative Disc Disease is a spinal condition caused by the breakdown of the intervertebral discs. As one ages, the spine begins to show signs of wear and tear because the discs dry out and shrink. These age-related changes can lead to arthritis, disc herniation, or spinal stenosis.

When the space between two adjacent vertebrae narrows, compression of a nerve root emerging from the spinal cord may result in radiculopathy (sensory and motor disturbances, such as severe pain in the neck, shoulder, arm, back, or leg, accompanied by muscle weakness). Less commonly, direct pressure on the spinal cord (typically in the cervical spine) may result in myelopathy, characterized by global weakness, gait dysfunction, loss of balance, and loss of bowel or bladder control. The patient may experience shocks (paresthesia) in hands and legs because of nerve compression and lack of blood flow. If vertebrae of the neck are involved it is labelled cervical spondylosis. Lower back spondylosis is labeled lumbar spondylosis.

In some embodiments, the methods disclosed herein can be used to treat degenerative disc disease. In some embodiments, the methods disclosed herein can be used to prevent further cartilage breakdown after a degenerative disc disease diagnosis. In some embodiments, the methods disclosed herein can be used to induce chondrocyte differentiation a degenerative disc disease diagnosis. Additional methods disclosed herein can be used to facilitate recovery after a degenerative disc disease diagnosis. In some embodiments, provided herein are methods of modifying the progression of degenerative disc disease.

Provided herein are methods for treating degenerative disc disease in a subject comprising administering to the subject a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof. Additionally, methods for treating degenerative disc disease are provided comprising administering to the subject a single compound or a pharmaceutically acceptable salt or solvate thereof, wherein the single compound is a dual DYRK/CLK inhibitor, which inhibits CLK and DYRK. In some embodiments, the methods for treating degenerative disk disease in a subject comprise administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. Additional methods for treating degenerative disc disease include comprising administering to the subject a single compound or a pharmaceutically acceptable salt or solvate thereof, wherein the single compound is a dual DYRK1A/CLK2 and/or CLK3 inhibitor, which inhibits DYRK1A and CLK2 and/or CLK3.

In some embodiments, the first compound, the second compound, and the dual DYRK/CLK inhibitor, are each independently selected from compounds of Formulas (I)-(VIII), or a pharmaceutically acceptable salt or solvate thereof.

### Meniscus Injury

The meniscus is a piece of cartilage in the knee that cushions and stabilizes the joint. Meniscus injury can occur as a result of participation in sports that require jumping, twisting, or changing direction suddenly while running. Meniscus injury may be more likely to occur in older people, because the meniscus weakens with age. In some embodiments, the methods disclosed herein can be used to treat meniscus injury. In some embodiments, the methods disclosed herein can be used to prevent further cartilage breakdown after a meniscus injury. In some embodiments, the methods disclosed herein can be used to induce chondrocyte differentiation after meniscus injury. Additional methods disclosed herein can be used to facilitate recovery after a meniscus injury.

Provided herein are methods for treating meniscus injury in a subject comprising administering to the subject a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof. Additionally, methods for treating meniscus injury are provided comprising administering to the subject a single compound or a pharmaceutically acceptable salt or solvate thereof, wherein the single compound is a dual DYRK/CLK inhibitor, which inhibits CLK and DYRK. In some embodiments, the methods for treating meniscus injury in a subject comprise administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. Additional methods for treating meniscus injury include comprising administering to the subject a single compound or a pharmaceutically acceptable salt or solvate thereof, wherein the single compound is a dual DYRK1A/CLK2 and/or CLK3 inhibitor, which inhibits DYRK1A and CLK2 and/or CLK3.

In some embodiments, the first compound, the second compound, and the dual DYRK/CLK inhibitor, are each independently selected from compounds of Formulas (I)-(VIII), or a pharmaceutically acceptable salt or solvate thereof.

### Relapsing Polychondritis

Relapsing polychondritis is a multi-systemic condition characterized by repeated episodes of inflammation and deterioration of cartilage. The often painful disease can cause joint deformity and be life-threatening if the respiratory tract, heart valves, or blood vessels are affected. In some embodiments, the methods disclosed herein can be used to treat relapsing polychondritis. In some embodiments, the methods disclosed herein can be used to prevent further cartilage breakdown after a relapsing polychondritis diagnosis. In some embodiments, the methods disclosed herein can be used to induce chondrocyte differentiation after a relapsing polychondritis diagnosis. Additional methods disclosed herein can be used to facilitate recovery after a relapsing polychondritis diagnosis. In some embodiments, provided herein are methods of modifying the progression of relapsing polychondritis.

Provided herein are methods for treating relapsing polychondritis in a subject comprising administering to the subject a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof. Additionally, methods for treating relapsing polychondritis are provided comprising administering to the subject a single compound or a pharmaceutically acceptable salt or solvate thereof, wherein the single compound is a dual DYRK/CLK inhibitor, which inhibits CLK and DYRK. In some embodiments, the methods for treating relapsing polychondritis in a subject comprise administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. Additional methods for treating relapsing polychondritis include comprising administering to the subject a single compound or a pharmaceutically acceptable salt or solvate thereof, wherein the single compound is a dual DYRK1A/CLK2 and/or CLK3 inhibitor, which inhibits DYRK1A and CLK2 and/or CLK3.

In some embodiments, the first compound, the second compound, and the dual DYRK/CLK inhibitor, are each independently selected from compounds of Formulas (I)-(VIII), or a pharmaceutically acceptable salt or solvate thereof.

### Rheumatoid Arthritis (RA)

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body.

Arthritis of joints involves inflammation of the synovial membrane. Joints become swollen, tender and warm, and stiffness limits their movement. With time, multiple joints are affected (polyarthritis). Most commonly involved are the small joints of the hands, feet and cervical spine, but larger joints like the shoulder and knee can also be involved. Synovitis can lead to tethering of tissue with loss of movement and erosion of the joint surface causing deformity and loss of function [The American Journal of Medicine (2007), 120(11), 936-939]

RA typically manifests with signs of inflammation, with the affected joints being swollen, warm, painful and stiff, particularly early in the morning on waking or following prolonged inactivity. Increased stiffness early in the morning is often a prominent feature of the disease and typically lasts for more than an hour. Gentle movements may relieve symptoms in early stages of the disease. These signs help distinguish rheumatoid from non-inflammatory problems of the joints, such as osteoarthritis. In arthritis of non-inflammatory causes, signs of inflammation and early morning stiffness are less prominent. The pain associated with RA is induced at the site of inflammation and classified as nociceptive as opposed to neuropathic. The joints are often affected in a fairly symmetrical fashion, although this is not specific, and the initial presentation may be asymmetrical.

As the pathology progresses the inflammatory activity leads to tendon tethering and erosion and destruction of the joint surface, which impairs range of movement and leads to deformity. The fingers may suffer from almost any deformity depending on which joints are most involved. Specific deformities, which also occur in osteoarthritis, include ulnar deviation, boutonniere deformity (also “buttonhole deformity”, flexion of proximal interphalangeal joint and extension of distal interphalangeal joint of the hand), swan neck deformity (hyperextension at proximal interphalangeal joint and flexion at distal interphalangeal joint) and “Z-thumb.” “Z-thumb” or “Z-deformity” consists of hyperextension of the interphalangeal joint, fixed flexion and subluxation of the metacarpophalangeal joint and gives a “Z” appearance to the thumb. The hammer toe deformity may be seen. In the worst case, joints are known as arthritis mutilans due to the mutilating nature of the deformities

In some embodiments, the methods disclosed herein can be used to treat rheumatoid arthritis. In some embodiments, the methods disclosed herein can be used to prevent further cartilage breakdown after rheumatoid arthritis diagnosis. In some embodiments, the methods disclosed herein can be used to induce chondrocyte differentiation after a rheumatoid arthritis diagnosis. Additional methods disclosed herein can be used to facilitate recovery after a rheumatoid arthritis diagnosis. In some embodiments, provided herein are methods of modifying the progression of rheumatoid arthritis.

Provided herein are methods for treating rheumatoid arthritis in a subject comprising administering to the subject a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof. Additionally, methods for treating rheumatoid arthritis are provided comprising administering to the subject a single compound or a pharmaceutically acceptable salt or solvate thereof, wherein the single compound inhibits is a dual DYRK/CLK inhibitor, which CLK and DYRK. In some embodiments, the methods for treating rheumatoid arthritis in a subject comprises administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. Additional methods for treating rheumatoid arthritis include administering to the subject a single compound or a pharmaceutically acceptable salt or solvate thereof, wherein the single compound is a dual DYRK1A/CLK2 and/or CLK3 inhibitor, which inhibits DYRK1A and CLK2 and/or CLK3.

In some embodiments, the first compound, the second compound, and the dual DYRK/CLK inhibitor, are each independently selected from compounds of Formulas (I)-(VIII), or a pharmaceutically acceptable salt or solvate thereof.

### Osteoarthritis

Osteoarthritis is a chronic degenerative joint disease in which cartilage and bone are primarily affected and for which acceptable long-term therapy does not yet exist. Osteoarthritis is especially common among people over 65 years of age, and usually affects a joint on one side of the body. In osteoarthritis, the cartilage breaks down and wears away, causing pain, swelling, and loss of motion of the joint. Osteoarthritis can affect any joint in the body, including one or more of the hands, feet, spine, shoulders, elbows, ankles, wrists, and the large weight bearing joints, such as the hips and knees. To date, clinical efforts aimed at treating osteoarthritis have been primarily directed toward symptomatic relief of pain and inflammation.

Provided herein are methods for treating osteoarthritis in a subject comprising administering to the subject a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof. Additionally, methods for treating osteoarthritis are provided comprising administering to the subject a single compound or a pharmaceutically acceptable salt or solvate thereof, wherein the single compound inhibits is a dual DYRK/CLK inhibitor, which CLK and DYRK. In some embodiments, the methods for treating osteoarthritis in a subject comprises administering to the subject a first compound, wherein the first compound is a CLK2 and/or CLK3 inhibitor, or a pharmaceutically acceptable salt or solvate thereof, and a second compound, wherein the second compound is a DYRK1A inhibitor, or a pharmaceutically acceptable salt or solvate thereof. Additional methods for treating osteoarthritis include administering to the subject a single compound or a pharmaceutically acceptable salt or solvate thereof, wherein the single compound is a dual DYRK1A/CLK2 and/or CLK3 inhibitor, which inhibits DYRK1A and CLK2 and/or CLK3. In some embodiments, the methods disclosed herein can be used to treat osteoarthritis. In some embodiments, the methods disclosed herein can be used to prevent further cartilage breakdown after rheumatoid osteoarthritis. In some embodiments, the methods disclosed herein can be used to induce chondrocyte differentiation after an osteoarthritis diagnosis. Additional methods disclosed herein can be used to facilitate recovery after an osteoarthritis diagnosis.

In some embodiments, the method comprises selecting a subject by detecting an elevated level of Wnt pathway activity in a sample from a subject, as compared to a reference level. In some embodiments, the method comprises modifying the progression of osteoarthritis by administering a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof. In some embodiments, the method comprises modifying the progression of osteoarthritis by administering a therapeutically effective amount of a single compound, wherein the single compound is a dual DYRK/CLK inhibitor, which inhibits CLK and DYRK. Accordingly, provided herein are methods of modifying the progression of osteoarthritis.

In some embodiments, the first compound, the second compound, and the dual DYRK/CLK inhibitor, are each independently selected from compounds of Formulas (I)-(VIII), or a pharmaceutically acceptable salt or solvate thereof.

### Disease Modifying Methods

In some embodiments, the methods provided herein can result in disease modification. Disease modification can refer to treatments or interventions that affect the underlying pathophysiology of the disease and have a beneficial outcome on the course or progression of the disease, for example, RA or osteoarthritis. Disease modification can also refer to interventions that modify or change the course of the disease, such as RA or osteoarthritis. Disease modification can also refer to interventions that slow down or reduce disease progression, for example, reducing or slowing down RA or osteoarthritis progression. Disease modification can also refer to interventions that stabilize disease progression, such as RA or osteoarthritis progression. In some embodiments, disease modification is stabilization in a particular stage of the disease, for example, stabilization of a subject at a particular RA or osteoarthritic stage. In some embodiments, disease modification is increased time in progression to a more severe stage of the disease, such as a more severe RA or osteoarthritic stage. In some embodiments, disease modification is stabilization in one or more subject reported symptoms. In some embodiments, disease modification is improvement in one or more subject reported symptoms. In some embodiments, disease modification is stabilization in one or more objective physical findings (e.g. physician monitored range of motion, or width, thickness, or volume of the cartilage, measurements of the space between bones, and levels of synovial fluid, etc.). In some embodiments, disease modification is improvement in one or more objective physical findings. In some embodiments, disease modification is stabilization in one or more inflammatory biomarkers discussed herein. In some embodiments, disease modification is a decrease in one or more inflammatory biomarkers discussed herein. In some embodiments, disease modification is decreasing Wnt pathway activation, such that the level of Wnt pathway activation is not elevated compared to a reference level.

Additionally, provided herein are methods of treating a subject that include first assessing the severity of the disease in the subject and then administering to the subject a first compound, or a pharmaceutically acceptable salt or solvate thereof, and a second compound, or a pharmaceutically acceptable salt or solvate thereof, based on the assessment. For example, administering a first compound and a second compound each independently selected from compounds of Formulas (I)-(VIII), or a pharmaceutically acceptable salt or solvate thereof. Also provided herein are methods of treating a subject that include first assessing the severity of the disease in the subject and then administering to the subject a single compound, or a pharmaceutically acceptable salt or solvate thereof, based on the assessment. For example, administering a compound of Formulas (I)-(VIII), or a pharmaceutically acceptable salt or solvate thereof. In some embodiments, the severity of the disorder is determined at one or more locations within a subject's body. For example, the severity of the disorder is determined at or near the target site of administration. In some embodiments, the osteoarthritis is present and assessed in one or more of the hands, feet, spine, shoulders, elbows, ankles, wrists, and the large weight bearing joints, such as the hips and knees.

The severity of a subject's osteoarthritis can be determined using a variety of methods. For example, radiological criteria (e.g., X-rays, CT scans, MRI, ultrasonography, and bone scanning), clinical criteria, pain assessments (e.g., visual analog scale (VAS) and Western Ontario and McMaster Universities Arthritis Index (WOMAC), Numeric Rating Scale (NRS), or Numeric Pain Rating Scale (NPRS) scores), mobility assessments (e.g., physician global assessments), thickness of cartilage (e.g., at the target site of administration), total volume of cartilage (e.g., at the target site of administration), levels of anabolic or catabolic biomarkers indicative of cartilage synthesis or degradation (e.g., cartilage oligomeric matrix protein [COMP], N-terminal propeptides of procollagen type I [PINP], and β-C-terminal telopeptide [β-CTX]), ARG8, COMP, PIANP, 5-ARGS, plasma levels of cytokines related to inflammation (interleukin [IL] 1b, IL6, IL8, tumor necrosis factor (TNF), and interferon-alpha [IFNα]), levels of bone marrow edema (e.g., by MRI scans of the target site of administration), levels of synovial fluid, clarity of synovial fluid (e.g., levels of crystals present in the fluid when viewed under a polarized microscope), levels of metalloproteinases (e.g., collagenase, stromelysin, MMPs, ADAMTS, etc.), levels of free radicals (e.g., nitric oxide), and measurements of the space between bones. In some embodiments, one or more methods of assessing the severity of a subject's osteoarthritis or disease state can be used.

Assessments of a joint can be made at one or more locations at, around, or near the joint. For example, multiple measurements of the width, thickness, or volume of the cartilage can be made. In some embodiments, the results of multiple measurements can be combined into a composite score which can be used to assess the severity of the disorder. Various methods of assessing the joint can also be considered together to determine the severity of the disorder. For example, subjective measurements such as pain and mobility determinations can be combined with objective measurements in one or more locations of the joint such as width, thickness, or volume of the cartilage, measurements of the space between bones, and levels of synovial fluid.

In some embodiments, the severity of the disease is determined based on the stage of the disorder. For example, osteoarthritis (OA) of the knee can be divided into five stages: 0 is assigned to a normal, healthy knee. The highest stage, 4, is assigned to severe OA. Exemplary diagnosis criteria and typical symptoms of the various stages are provided below in Table 1.

TABLE 1

| Stage | Symptoms |
|---|---|
| 0 | Stage 0 OA is classified as “normal” knee health. The knee joint shows no signs of |
| | OA, and the joint functions without any impairment or pain. |
| 1 | A person with stage 1 OA is showing very minor bone spur growth (bone spurs are |
| | boney growths that often develop where bones meet each other in the joint). Likely, |
| | a person with stage 1 OA is not experiencing any pain or discomfort as a result of |
| | the very minor wear on the components of the joint. |
| 2 | Stage 2 OA of the knee is considered a “mild” stage of the condition. X-rays of knee |
| | joints in this stage will likely reveal greater bone spur growth, but the cartilage likely |
| | remains at a healthy size - the space between the bones is normal, and the bones |
| | are not rubbing or scraping one another. Synovial fluid is also typically still present |
| | at sufficient levels for normal joint motion. However, this is the stage where people |
| | may first begin experiencing symptoms - pain after a long day of walking or |
| | running, greater stiffness in the joint when it's not used for several hours, tenderness |
| | when kneeling or bending. |
| 3 | Stage 3 OA is classified as “moderate” OA. The cartilage between bones is showing |
| | obvious damage, and the space between the bones is narrowing. People with stage |
| | 3 OA of the knee are likely experiencing frequent pain when walking, running, |
| | bending, or kneeling. They also may experience joint stiffness after sitting for long |
| | periods of time or when waking up in the morning. Joint swelling may be present |
| | after extended periods of motion, too. |
| 4 | Stage 4 OA is considered “severe.” People in stage 4 OA of the knee experience |
| | great pain and discomfort when walking or moving the joint. The joint space |
| | between bones is dramatically reduced - the cartilage is almost completely gone, |
| | leaving the joint stiff and possibly immobile. The synovial fluid is decreased |
| | dramatically, and it no longer helps reduce the friction among the moving parts of a |
| | joint. |

Similarly, the stages of hip osteoarthritis can divided into five stages according to the severity observed in various images. Exemplary diagnosis criteria and typical symptoms of the various stages are provided below in Table 2.

TABLE 2

| Stage | Plain film grading | MRI grading |
|---|---|---|
| 0 | Normal | Normal |
| 1 | Possible joint space narrowing and | Inhomogeneous high signal intensity in |
| | subtle osteophytes | cartilage (T2WI) |
| 2 | Definite joint space narrowing, | Inhomogeneity with areas of high signal |
| | defined osteophytes and some | intensity in articular cartilage (T2WI); |
| | sclerosis, especially in acetabular | indistinct trabeculae or signal intensity |
| | region | loss in femoral head & neck (T1WI) |
| 3 | Marked joint space narrowing, small | Criteria of Stage 1 & 2 plus indistinct |
| | osteophytes, some sclerosis and cyst | zone between femoral head & |
| | formation and deformity of femoral | acetabulum; subchondral signal loss due |
| | head and acetabulum | to bone sclerosis |
| 4 | Gross loss of joint space with above | Above criteria plus femoral head |
| | features plus large osteophytes and | deformity |
| | increased deformity of the femoral | |
| | head and acetabulum | |

In some embodiments, a first compound, wherein the first compound is a CLK inhibitor, or a pharmaceutically acceptable salt or solvate thereof, and a second compound, wherein the second compound is a DYRK inhibitor, or a pharmaceutically acceptable salt or solvate thereof, can be used to treat osteoarthritis in combination with any of the following compounds and/or methods: (a) Nonsteroidal anti-inflammatory drugs (NSAIDs) such as ibuprofen, naproxen, aspirin and acetaminophen; (b) physical therapy; (c) injections of corticosteroid medications; (d) injections of hyaluronic acid derivatives (e.g. Hyalgan, Synvisc); (e) narcotics, like codeine; (f) braces and/or shoe inserts or any device that can immobilize or support your joint to help you keep pressure off it (e.g., splints, braces, shoe inserts or other medical devices); (g) realigning bones (osteotomy); (h) joint replacement (arthroplasty); and (i) a course for dealing with chronic pain.

In some embodiments, a single compound, wherein the single compound, or a pharmaceutically acceptable salt or solvate thereof, inhibits DYRK and CLK can be used to treat osteoarthritis in combination with any of the following compounds and/or methods: (a) Nonsteroidal anti-inflammatory drugs (NSAIDs) such as ibuprofen, naproxen, aspirin and acetaminophen; (b) physical therapy; (c) injections of corticosteroid medications; (d) injections of hyaluronic acid derivatives (e.g. Hyalgan, Synvisc); (e) narcotics, like codeine; (f) braces and/or shoe inserts or any device that can immobilize or support your joint to help you keep pressure off it (e.g., splints, braces, shoe inserts or other medical devices); (g) realigning bones (osteotomy); (h) joint replacement (arthroplasty); and (i) a course for dealing with chronic pain. In other embodiments, a single compound can be used to treat osteoarthritis in combination with any of the following methods: (a) Nonsteroidal anti-inflammatory drugs (NSAIDs) such as ibuprofen, naproxen, aspirin and acetaminophen; (b) physical therapy; (c) injections of corticosteroid medications; (d) injections of hyaluronic acid derivatives (e.g. Hyalgan, Synvisc); (e) narcotics, like codeine; (f) braces and/or shoe inserts or any device that can immobilize or support your joint to help you keep pressure off it (e.g., splints, braces, shoe inserts or other medical devices); (g) realigning bones (osteotomy); (h) joint replacement (arthroplasty); and (i) in combination with a chronic pain class.

### Cartilage Growth

In some embodiments, administration of one or more compounds and methods provided herein promote increased cartilage growth. Assessments of a joint can be made at one or more locations at, around, or near the joint. In some embodiments, cartilage growth is measured by cartilage thickness. In some embodiments, cartilage growth is measured by cartilage width. In some embodiments, cartilage growth is measured by volume of the cartilage. In some embodiments, the results of multiple measurements can be combined into a composite score which can be used to assess the severity of the disorder. Various methods of assessing the joint can also be considered together to determine the severity of the disorder. For example, subjective measurements such as pain and mobility determinations can be combined with objective measurements in one or more locations of the joint such as width, thickness, or volume of the cartilage, measurements of the space between bones, and levels of synovial fluid.

### Selecting a Subject, Assessing Joint Damage, and Assessing Method Efficacy

As used herein, methods described herein comprise selecting a subject. In some embodiments, a subject is selected using a variety of techniques. For example, radiological criteria (e.g., X-rays, CT scans, MRI, ultrasonography, and bone scanning), clinical criteria, pain assessments (e.g., visual analog scale (VAS) and Western Ontario and McMaster Universities Arthritis Index (WOMAC), Numeric Rating Scale (NRS), or Numeric Pain Rating Scale (NPRS) scores), mobility assessments (e.g., physician global assessments), thickness of cartilage (e.g., at the target site of administration), total volume of cartilage (e.g., at the target site of administration), levels of anabolic or catabolic biomarkers indicative of cartilage synthesis or degradation (e.g., cartilage oligomeric matrix protein [COMP], N-terminal propeptides of procollagen type I [PINP], and beta-C-terminal telopeptide [β-CTX]), ARG8, COMP, PIANP, 5-ARGS, plasma levels of cytokines related to inflammation (interleukin [IL] 1β, IL6, IL8, tumor necrosis factor (TNF), and interferon-alpha [IFN-α]), levels of bone marrow edema (e.g., by MRI scans of the target site of administration), levels of synovial fluid, clarity of synovial fluid (e.g., levels of crystals present in the fluid when viewed under a polarized microscope), levels of metalloproteinases (e.g., collagenase, stromelysin, MMPs, ADAMTS, etc.), ARGS, ADAMTS5, levels of free radicals (e.g., nitric oxide), and measurements of the space between bones. In some embodiments, a subject is selected based on joint inflammation. In some embodiments, a subject is selected based on the extent of joint effusion. In some embodiments, the subject is selected based on radiological criteria in combination with pain scores. In some embodiments, the subject is selected based on biomarker levels and pain scores. In some embodiments, the subject is selected based on clinical criteria and biomarker levels. In some embodiments, one or more techniques of assessing the severity of a subject's disease or condition can be used. In some embodiments, the methods require 2 to 4 mm of baseline cartilage.

Additionally, provided herein are methods of treating a subject that include first assessing the severity of the disease in the subject and then administering to the subject a first compound and a second compound based on the assessment. Also provided herein are methods of treating a subject that include first assessing the severity of the disease in the subject and then administering to the subject a single compound based on the assessment. In some embodiments, the severity of the disorder is determined at one or more locations within a subject's body. For example, the severity of the disorder is determined at or near the target site of administration. Assessments of a joint can be made at one or more locations at, around, or near the joint. For example, multiple measurements of the width, thickness, or volume of the cartilage can be made. In some embodiments, the results of multiple measurements can be combined into a composite score which can be used to assess the severity of the disorder. Various methods of assessing the joint can also be considered together to determine the severity of the disorder. For example, subjective measurements such as pain and mobility determinations can be combined with objective measurements in one or more locations of the joint such as width, thickness, or volume of the cartilage, measurements of the space between bones, and levels of synovial fluid.

In some embodiments, one or more techniques of assessing the methods' efficacy can be used. The one or more techniques of assessing the method's efficacy can be the same as the techniques used to select a subject for treatment. The one or more methods of assessing the method's efficacy can be different from the techniques used to select a subject for treatment. The one or more techniques of assessing the method's efficacy can be the same as the techniques used to assess the severity of a subject's disease or condition. The one or more methods of assessing the method's efficacy can be different from the techniques used to assess the severity of a subject's disease or condition.

### CLK Family

The CLK family kinases are an evolutionarily conserved group of dual specificity kinases, capable of phosphorylating protein substrates on serine, threonine, and tyrosine residues. The CLK family contains four members (CLK1, CLK2, CLK3 and CLK4). CLKs are proposed to exert their function by directly phosphorylating serine and arginine rich splicing factor (SRSF) proteins. SRSFs are reported to play an important role in spliceosome assembly and regulation of alternative splicing and gene expression.

Exemplary human CLK1, CLK2, CLK3, and CLK4 protein sequences are SEQ ID NO: 1, 3, 5, 7, 9, 11, 13, 15, and 17. Exemplary cDNA sequences that encode CLK1, CLK2, CLK3, and CLK4 are SEQ ID NO: 2, 4, 6, 8, 10, 12, 14, 16, and 18.

Human CLK1 protein isoform 1
(SEQ ID NO: 1)
MRHSKRTYCPDWDDKDWDYGKWRSSSSHKRRKRSHSSAQENKRCKY
NHSKMCDSHYLESRSINEKDYHSRRYIDEYRNDYTQGCEPGHRQRD
HESRYQNHSSKSSGRSGRSSYKSKHRIHHSTSHRRSHGKSHRRK
RTRSVEDDEEGHLICQSGDVLSARYEIVDTLGEGAFGKVVECID
HKAGGRHVAVKIVKNVDRYCEAARSEIQVLEHLNTTDPNSTFRC
VQMLEWFEHHGHICIVFELLGLSTYDFIKENGFLPFRLDHIRKM
AYQICKSVNFLHSNKLTHTDLKPENILFVQSDYTEAYNPKIKRD
ERTLINPDIKVVDFGSATYDDEHHSTLVSTRHYRAPEVILALGW
SQPCDVWSIGCILIEYYLGFTVFPTHDSKEHLAMMERILGPLPK
HMIQKTRKRKYFHHDRLDWDEHSSAGRYVSRRCKPLKEFMLSQD
VEHERLFDLIQKMLEYDPAKRITLREALKHPFFDLLKKSI
Human CLK1 cDNA isoform 1
(SEQ ID NO: 2)
atgagacac tcaaagagaa cttactgtcc tgattgggat
gacaaggatt gggattatgg aaaatggagg
agcagcagca gtcataaaag aaggaagaga tcacatagca
gtgcccagga gaacaagcgc tgcaaataca atcactctaa
aatgtgtgat agccattatt tggaaagcag gtctataaat
gagaaagatt atcatagtcg acgctacatt gatgagtaca
gaaatgacta cactcaagga tgtgaacctg gacatcgcca
aagagaccat gaaagccggt atcagaacca tagtagcaag
tcttctggta gaagtggaag aagtagttat aaaagcaaac
acaggattca ccacagtact tcacatcgtc gttcacatgg
gaagagtcac cgaaggaaaa gaaccaggag tgtagaggat
gatgaggagg gtcacctgat ctgtcagagt ggagacgtac
taagtgcaag atatgaaatt gttgatactt taggtgaagg
agcttttgga aaagttgtgg agtgcatcga tcataaagcg
ggaggtagac atgtagcagt aaaaatagtt aaaaatgtgg
atagatactg tgaagctgct cgctcagaaa tacaagttct
ggaacatctg aatacaacag accccaacag tactttccgc
tgtgtccaga tgttggaatg gtttgagcat catggtcaca
tttgcattgt ttttgaacta ttgggactta gtacttacga
cttcattaaa gaaaatggtt ttctaccatt tcgactggat
catatcagaa agatggcata tcagatatgc aagtctgtga
attttttgca cagtaataag ttgactcaca cagacttaaa
gcctgaaaac atcttatttg tgcagtctga ctacacagag
gcgtataatc ccaaaataaa acgtgatgaa cgcaccttaa
taaatccaga tattaaagtt gtagactttg gtagtgcaac
atatgatgac gaacatcaca gtacattggt atctacaaga
cattatagag cacctgaagt tattttagcc ctagggtggt
cccaaccatg tgatgtctgg agcataggat gcattcttat
tgaatactat cttgggttta ccgtatttcc aacacacgat
agtaaggagc atttagcaat gatggaaagg attcttggac
ctctaccaaa acatatgata cagaaaacca ggaaacgtaa
atattttcac cacgatcgat tagactggga tgaacacagt
tctgccggca gatatgtttc aagacgctgt aaacctctga
aggaatttat gctttctcaa gatgttgaac atgagcgtct
ctttgacctc attcagaaaa tgttggagta tgatccagcc
aaaagaatta ctctcagaga agccttaaag catcctttct
ttgaccttct gaagaaaagt atatag
Human CLK1 protein isoform 2
(SEQ ID NO: 3)
MAAGRRPASALWPERRGSPLRGDLLGFQNVREPSSCGETLSGMR
HSKRTYCPDWDDKDWDYGKWRSSSSHKRRKRSHSSAQENKRCKYN
HSKMCDSHYLESRSINEKDYHSRRYIDEYRNDYTQGCEPGHRQRD
HESRYQNHSSKSSGRSGRSSYKSKHRIHHSTSHRRSHGKSHRRKR
TRSVEDDEEGHLICQSGDVLSARYEIVDTLGEGAFGKVVECIDHK
AGGRHVAVKIVKNVDRYCEAARSEIQVLEHLNTTDPNSTFRCVQ
MLEWFEHHGHICIVFELLGLSTYDFIKENGFLPFRLDHIRKMAY
QICKSVNFLHSNKLTHTDLKPENILFVQSDYTEAYNPKIKRDER
TLINPDIKVVDFGSATYDDEHHSTLVSTRHYRAPEVILALGWSQ
PCDVWSIGCILIEYYLGFTVFPTHDSKEHLAMMERILGPLPKHM
IQKTRKRKYFHHDRLDWDEHSSAGRYVSRRCKPLKEFMLSQDVE
HERLFDLIQKMLEYDPAKRITLREALKHPFFDLLKKSI
Human CLK1 cDNA isoform 2
(SEQ ID NO: 4)
atggc ggctgggcgg aggccggctt cggccctgtg
gccggaaagg cgaggctccc cgttgagggg
ggatttgctg gggttccaga atgtgcgtga gccaagcagc
tgtggggaaa cgttgtctgg aatgagacac tcaaagagaa
cttactgtcc tgattgggat gacaaggatt gggattatgg
aaaatggagg agcagcagca gtcataaaag aaggaagaga
tcacatagca gtgcccagga gaacaagcgc tgcaaataca
atcactctaa aatgtgtgat agccattatt tggaaagcag
gtctataaat gagaaagatt atcatagtcg acgctacatt
gatgagtaca gaaatgacta cactcaagga tgtgaacctg
gacatcgcca aagagaccat gaaagccggt atcagaacca
tagtagcaag tcttctggta gaagtggaag aagtagttat
aaaagcaaac acaggattca ccacagtact tcacatcgtc
gttcacatgg gaagagtcac cgaaggaaaa gaaccaggag
tgtagaggat gatgaggagg gtcacctgat ctgtcagagt
ggagacgtac taagtgcaag atatgaaatt gttgatactt
taggtgaagg agcttttgga aaagttgtgg agtgcatcga
tcataaagcg ggaggtagac atgtagcagt aaaaatagtt
aaaaatgtgg atagatactg tgaagctgct cgctcagaaa
tacaagttct ggaacatctg aatacaacag accccaacag
tactttccgc tgtgtccaga tgttggaatg gtttgagcat
catggtcaca tttgcattgt ttttgaacta ttgggactta
gtacttacga cttcattaaa gaaaatggtt ttctaccatt
tcgactggat catatcagaa agatggcata tcagatatgc
aagtctgtga attttttgca cagtaataag ttgactcaca
cagacttaaa gcctgaaaac atcttatttg tgcagtctga
ctacacagag gcgtataatc ccaaaataaa acgtgatgaa
cgcaccttaa taaatccaga tattaaagtt gtagactttg
gtagtgcaac atatgatgac gaacatcaca gtacattggt
atctacaaga cattatagag cacctgaagt tattttagcc
ctagggtggt cccaaccatg tgatgtctgg agcataggat
gcattcttat tgaatactat cttgggttta ccgtatttcc
aacacacgat agtaaggagc atttagcaat gatggaaagg
attcttggac ctctaccaaa acatatgata cagaaaacca
ggaaacgtaa atattttcac cacgatcgat tagactggga
tgaacacagt tctgccggca gatatgtttc aagacgctgt
aaacctctga aggaatttat gctttctcaa gatgttgaac
atgagcgtct ctttgacctc attcagaaaa tgttggagta
tgatccagcc aaaagaatta ctctcagaga agccttaaag
catcctttct ttgaccttct gaagaaaagt atatag
Human CLK2 protein isoform 1
(SEQ ID NO: 5)
MPHPRRYHSSERGSRGSYREHYRSRKHKRRRSRSWSSSSDRTRR
RRREDSYHVRSRSSYDDRSSDRRVYDRRYCGSYRRNDYSRDRGD
AYYDTDYRHSYEYQRENSSYRSQRSSRRKHRRRRRRSRTFSRSS
SQHSSRRAKSVEDDAEGHLIYHVGDWLQERYEIVSTLGEGTFGR
VVQCVDHRRGGARVALKIIKNVEKYKEAARLEINVLEKINEKDP
DNKNLCVQMFDWFDYHGHMCISFELLGLSTFDFLKDNNYLPYPI
HQVRHMAFQLCQAVKFLHDNKLTHTDLKPENILFVNSDYELTYN
LEKKRDERSVKSTAVRVVDFGSATFDHEHHSTIVSTRHYRAPEV
ILELGWSQPCDVWSIGCIIFEYYVGFTLFQTHDNREHLAMMERI
LGPIPSRMIRKTRKQKYFYRGRLDWDENTSAGRYVRENCKPLRR
YLTSEAEEHHQLFDLIESMLEYEPAKRLTLGEALQHPFFARLRA
EPPNKLWDSSRDISR
Human CLK2 cDNA isoform 1
(SEQ ID NO: 6)
a tgccgcatcc tcgaaggtac cactcctcag
agcgaggcag ccgggggagt taccgtgaac actatcggag
ccgaaagcat aagcgacgaa gaagtcgctc ctggtcaagt
agtagtgacc ggacacgacg gcgtcggcga gaggacagct
accatgtccg ttctcgaagc agttatgatg atcgttcgtc
cgaccggagg gtgtatgacc ggcgatactg tggcagctac
agacgcaacg attatagccg ggatcgggga gatgcctact
atgacacaga ctatcggcat tcctatgaat atcagcggga
gaacagcagt taccgcagcc agcgcagcag ccggaggaag
cacagacggc ggaggaggcg cagccggaca tttagccgct
catcttcgca gcacagcagc cggagagcca agagtgtaga
ggacgacgct gagggccacc tcatctacca cgtcggggac
tggctacaag agcgatatga aatcgttagc accttaggag
aggggacctt cggccgagtt gtacaatgtg ttgaccatcg
caggggtggg gctcgagttg ccctgaagat cattaagaat
gtggagaagt acaaggaagc agctcgactt gagatcaacg
tgctagagaa aatcaatgag aaagaccctg acaacaagaa
cctctgtgtc cagatgtttg actggtttga ctaccatggc
cacatgtgta tctcctttga gcttctgggc cttagcacct
tcgatttcct caaagacaac aactacctgc cctaccccat
ccaccaagtg cgccacatgg ccttccagct gtgccaggct
gtcaagttcc tccatgataa caagctgaca catacagacc
tcaagcctga aaatattctg tttgtgaatt cagactatga
gctcacctac aacctagaga agaagcgaga tgagcgcagt
gtgaagagca cagctgtgcg ggtggtagac tttggcagtg
ccacctttga ccatgagcac catagcacca ttgtctccac
tcgccattac cgagcaccag aagtcatcct tgagttgggc
tggtcacagc cttgtgatgt gtggagtata ggctgcatca
tctttgaata ctatgtggga ttcaccctct tccagaccca
tgacaacaga gagcatctag ccatgatgga aaggatcttg
ggtcctatcc cttcccggat gatccgaaag acaagaaagc
agaaatatttt taccggggt cgcctggatt gggatgagaa
cacatcagct gggcgctatg ttcgtgagaa ctgcaaaccg
ctgcggcggt atctgacctc agaggcagag gaacaccacc
agctcttcga tctgattgaa agcatgctag agtatgaacc
agctaagcgg ctgaccttgg gtgaagccct tcagcatcct
ttcttcgccc gccttcgggc tgagccgccc aacaagttgt
gggactccag tcgggatatc agtcggtga
Human CLK2 protein isoform 2
(SEQ ID NO: 7)
MPHPRRYHSSERGSRGSYREHYRSRKHKRRRSRSWSSSSDRTRR
RRREDSYHVRSRSSYDDRSSDRRVYDRRYCGSYRRNDYSRDRGD
AYYDTDYRHSYEYQRENSSYRSQRSSRRKHRRRRRRSRTFSRSS
SHSSRRAKSVEDDAEGHLIYHVGDWLQERYEIVSTLGEGTFGRV
VQCVDHRRGGARVALKIIKNVEKYKEAARLEINVLEKINEKDPD
NKNLCVQMFDWFDYHGHMCISFELLGLSTFDFLKDNNYLPYPIH
QVRHMAFQLCQAVKFLHDNKLTHTDLKPENILFVNSDYELTYNL
EKKRDERSVKSTAVRVVDFGSATFDHEHHSTIVSTRHYRAPEVI
LELGWSQPCDVWSIGCIIFEYYVGFTLFQTHDNREHLAMMERIL
GPIPSRMIRKTRKQKYFYRGRLDWDENTSAGRYVRENCKPLRRY
LTSEAEEHHQLFDLIESMLEYEPAKRLTLGEALQHPFFARLRAE
PPNKLWDSSRDISR
Human CLK2 cDNA isoform 2
(SEQ ID NO: 8)
a tgccgcatcc tcgaaggtac cactcctcag
agcgaggcag ccgggggagt taccgtgaac actatcggag
ccgaaagcat aagcgacgaa gaagtcgctc ctggtcaagt
agtagtgacc ggacacgacg gcgtcggcga gaggacagct
accatgtccg ttctcgaagc agttatgatg atcgttcgtc
cgaccggagg gtgtatgacc ggcgatactg tggcagctac
agacgcaacg attatagccg ggatcgggga gatgcctact
atgacacaga ctatcggcat tcctatgaat atcagcggga
gaacagcagt taccgcagcc agcgcagcag ccggaggaag
cacagacggc ggaggaggcg cagccggaca tttagccgct
catcttcgca cagcagccgg agagccaaga gtgtagagga
cgacgctgag ggccacctca tctaccacgt cggggactgg
ctacaagagc gatatgaaat cgttagcacc ttaggagagg
ggaccttcgg ccgagttgta caatgtgttg accatcgcag
gggtggggct cgagttgccc tgaagatcat taagaatgtg
gagaagtaca aggaagcagc tcgacttgag atcaacgtgc
tagagaaaat caatgagaaa gaccctgaca acaagaacct
ctgtgtccag atgtttgact ggtttgacta ccatggccac
atgtgtatct cctttgagct tctgggcctt agcaccttcg
atttcctcaa agacaacaac tacctgccct accccatcca
ccaagtgcgc cacatggcct tccagctgtg ccaggctgtc
aagttcctcc atgataacaa gctgacacat acagacctca
agcctgaaaa tattctgttt gtgaattcag actatgagct
cacctacaac ctagagaaga agcgagatga gcgcagtgtg
aagagcacag ctgtgcgggt ggtagacttt ggcagtgcca
cctttgacca tgagcaccat agcaccattg tctccactcg
ccattaccga gcaccagaag tcatccttga gttgggctgg
tcacagcctt gtgatgtgtg gagtataggc tgcatcatct
ttgaatacta tgtgggattc accctcttcc agacccatga
caacagagag catctagcca tgatggaaag gatcttgggt
cctatccctt cccggatgat ccgaaagaca agaaagcaga
aatattttta ccggggtcgc ctggattggg atgagaacac
atcagctggg cgctatgttc gtgagaactg caaaccgctg
cggcggtatc tgacctcaga ggcagaggaa caccaccagc
tcttcgatct gattgaaagc atgctagagt atgaaccagc
taagcggctg accttgggtg aagcccttca gcatcctttc
ttcgcccgcc ttcgggctga gccgcccaac aagttgtggg
actccagtcg ggatatcagt cggtga
Human CLK2 protein isoform 3
(SEQ ID NO: 9)
MFDWFDYHGHMCISFELLGLSTFDFLKDNNYLPYPIHQVRHMAF
QLCQAVKFLHDNKLTHTDLKPENILFVNSDYELTYNLEKKRDER
SVKSTAVRVVDFGSATFDHEHHSTIVSTRHYRAPEVILELGWSQ
PCDVWSIGCIIFEYYVGFTLFQTHDNREHLAMMERILGPIPSRM
IRKTRKQKYFYRGRLDWDENTSAGRYVRENCKPLRRYLTSEAEE
HHQLFDLIESMLEYEPAKRLTLGEALQHPFFARLRAEPPNKLWD
SSRDISR
Human CLK2 cDNA isoform 3
(SEQ ID NO: 10)
atgtt tgactggttt gactaccatg gccacatgtg
tatctccttt gagcttctgg gccttagcac cttcgatttc
ctcaaagaca acaactacct gccctacccc atccaccaag
tgcgccacat ggccttccag ctgtgccagg ctgtcaagtt
cctccatgat aacaagctga cacatacaga cctcaagcct
gaaaatattc tgtttgtgaa ttcagactat gagctcacct
acaacctaga gaagaagcga gatgagcgca gtgtgaagag
cacagctgtg cgggtggtag actttggcag tgccaccttt
gaccatgagc accatagcac cattgtctcc actcgccatt
accgagcacc agaagtcatc cttgagttgg gctggtcaca
gccttgtgat gtgtggagta taggctgcat catctttgaa
tactatgtgg gattcaccct cttccagacc catgacaaca
gagagcatct agccatgatg gaaaggatct tgggtcctat
cccttcccgg atgatccgaa agacaagaaa gcagaaatat
ttttaccggg gtcgcctgga ttgggatgag aacacatcag
ctgggcgcta tgttcgtgag aactgcaaac cgctgcggcg
gtatctgacc tcagaggcag aggaacacca ccagctcttc
gatctgattg aaagcatgct agagtatgaa ccagctaagc
ggctgacctt gggtgaagcc cttcagcatc ctttcttcgc
ccgccttcgg gctgagccgc ccaacaagtt gtgggactcc
agtcgggata tcagtcggtg a
Human CLK2 protein isoform 4
(SEQ ID NO: 11)
MPHPRRYHSSERGSRGSYREHYRSRKHKRRRSRSWSSSSDRTRR
RRREDSYHVRSRSYDDRSSDRRVYDRRYCGSYRRNDYSRDRGDA
YYDTDYRHSYEYQRENSSYRSQRSSRRKHRRRRRRSRTFSRSSS
HSSRRAKSVEDDAEGHLIYHVGDWLQERYEIVSTLGEGTFGRVV
QCVDHRRGGARVALKIIKNVEKYKEAARLEINVLEKINEKDPDN
KNLCVQMFDWFDYHGHMCISFELLGLSTFDFLKDNNYLPYPIHQ
VRHMAFQLCQAVKFLHDNKLTHTDLKPENILFVNSDYELTYNLE
KKRDERSVKSTAVRVVDFGSATFDHEHHSTIVSTRHYRAPEVIL
ELGWSQPCDVWSIGCIIFEYYVGFTLFQTHDNREHLAMMERILG
PIPSRMIRKTRKQKYFYRGRLDWDENTSAGRYVRENCKPLRRYL
TSEAEEHHQLFDLIESMLEYEPAKRLTLGEALQHPFFARLRAEP
PNKLWDSSRDISR
Human CLK2 cDNA isoform 4
(SEQ ID NO: 12)
a tgccgcatcc tcgaaggtac cactcctcag
agcgaggcag ccgggggagt taccgtgaac actatcggag
ccgaaagcat aagcgacgaa gaagtcgctc ctggtcaagt
agtagtgacc ggacacgacg gcgtcggcga gaggacagct
accatgtccg ttctcgaagt tatgatgatc gttcgtccga
ccggagggtg tatgaccggc gatactgtgg cagctacaga
cgcaacgatt atagccggga tcggggagat gcctactatg
acacagacta tcggcattcc tatgaatatc agcgggagaa
cagcagttac cgcagccagc gcagcagccg gaggaagcac
agacggcgga ggaggcgcag ccggacattt agccgctcat
cttcgcacag cagccggaga gccaagagtg tagaggacga
cgctgagggc cacctcatct accacgtcgg ggactggcta
caagagcgat atgaaatcgt tagcacctta ggagagggga
ccttcggccg agttgtacaa tgtgttgacc atcgcagggg
tggggctcga gttgccctga agatcattaa gaatgtggag
aagtacaagg aagcagctcg acttgagatc aacgtgctag
agaaaatcaa tgagaaagac cctgacaaca agaacctctg
tgtccagatg tttgactggt ttgactacca tggccacatg
tgtatctcct ttgagcttct gggccttagc accttcgatt
tcctcaaaga caacaactac ctgccctacc ccatccacca
agtgcgccac atggccttcc agctgtgcca ggctgtcaag
ttcctccatg ataacaagct gacacataca gacctcaagc
ctgaaaatat tctgtttgtg aattcagact atgagctcac
ctacaaccta gagaagaagc gagatgagcg cagtgtgaag
agcacagctg tgcgggtggt agactttggc agtgccacct
ttgaccatga gcaccatagc accattgtct ccactcgcca
ttaccgagca ccagaagtca tccttgagtt gggctggtca
cagccttgtg atgtgtggag tataggctgc atcatctttg
aatactatgt gggattcacc ctcttccaga cccatgacaa
cagagagcat ctagccatga tggaaaggat cttgggtcct
atcccttccc ggatgatccg aaagacaaga aagcagaaat
atttttaccg gggtcgcctg gattgggatg agaacacatc
agctgggcgc tatgttcgtg agaactgcaa accgctgcgg
cggtatctga cctcagaggc agaggaacac caccagctct
tcgatctgat tgaaagcatg ctagagtatg aaccagctaa
gcggctgacc ttgggtgaag cccttcagca tcctttcttc
gcccgccttc gggctgagcc gcccaacaag ttgtgggact
ccagtcggga tatcagtcgg tga
Human CLK3 protein isoform 1
(SEQ ID NO: 13)
MPVLSARRRELADHAGSGRRSGPSPTARSGPHLSALRAQPARAA
HLSGRGTYVRRDTAGGGPGQARPLGPPGTSLLGRGARRSGEGWC
PGAFESGARAARPPSRVEPRLATAASREGAGLPRAEVAAGSGRG
ARSGEWGLAAAGAWETMHHCKRYRSPEPDPYLSYRWKRRRSYSR
EHEGRLRYPSRREPPPRRSRSRSHDRLPYQRRYRERRDSDTYRC
EERSPSFGEDYYGPSRSRHRRRSRERGPYRTRKHAHHCHKRRTR
SCSSASSRSQQSSKRSSRSVEDDKEGHLVCRIGDWLQERYEIVG
NLGEGTFGKVVECLDHARGKSQVALKIIRNVGKYREAARLEINV
LKKIKEKDKENKFLCVLMSDWFNFHGHMCIAFELLGKNTFEFLK
ENNFQPYPLPHVRHMAYQLCHALRFLHENQLTHTDLKPENILFV
NSEFETLYNEHKSCEEKSVKNTSIRVADFGSATFDHEHHTTIVA
TRHYRPPEVILELGWAQPCDVWSIGCILFEYYRGFTLFQTHENR
EHLVMMEKILGPIPSHMIHRTRKQKYFYKGGLVWDENSSDGRYV
KENCKPLKSYMLQDSLEHVQLFDLMRRMLEFDPAQRITLAEALL
HPFFAGLT PEERSFHTSRNPSR
Human CLK3 cDNA isoform 1
(SEQ ID NO: 14)
a atgcccgtc ctctccgcgc gcaggaggga gttggcggac
cacgcggggt cggggcgacg gagcgggccc agccccacgg
ccaggtcggg gccccacctc tcggctctga gagcccagcc
ggcccgggcc gcgcacctgt caggtcgggg gacctacgtg
cgccgcgaca cggcgggagg cgggccgggc caggctcgtc
ccctcggccc tcccggaact agtctcctag gccgcggcgc
ccgccggagc ggagagggct ggtgccccgg agccttcgag
tcgggggcta gagcggccag gcctccgagc cgggtcgagc
cgaggctggc gacggctgcg tcacgcgagg gggcggggct
gccacgggcg gaggtcgcag ccggaagcgg aagaggcgct
cggagcgggg agtggggcct agctgcagcc ggagcctggg
agacgatgca tcactgtaag cgataccgct cccctgaacc
agacccgtac ctgagctacc gatggaagag gaggaggtcc
tacagtcggg aacatgaagg gagactgcga tacccgtccc
gaagggagcc tcccccacga agatctcggt ccagaagcca
tgaccgcctg ccctaccaga ggaggtaccg ggagcgccgt
gacagcgata cataccggtg tgaagagcgg agcccatcct
ttggagagga ctactatgga ccttcacgtt ctcgtcatcg
tcggcgatcg cgggagaggg ggccataccg gacccgcaag
catgcccacc actgccacaa acgccgcacc aggtcttgta
gcagcgcctc ctcgagaagc caacagagca gtaagcgcag
cagccggagt gtggaagatg acaaggaggg tcacctggtg
tgccggatcg gcgattggct ccaagagcga tatgagattg
tggggaacct gggtgaaggc acctttggca aggtggtgga
gtgcttggac catgccagag ggaagtctca ggttgccctg
aagatcatcc gcaacgtggg caagtaccgg gaggctgccc
ggctagaaat caacgtgctc aaaaaaatca aggagaagga
caaagaaaac aagttcctgt gtgtcttgat gtctgactgg
ttcaacttcc acggtcacat gtgcatcgcc tttgagctcc
tgggcaagaa cacctttgag ttcctgaagg agaataactt
ccagccttac cccctaccac atgtccggca catggcctac
cagctctgcc acgcccttag atttctgcat gagaatcagc
tgacccatac agacttgaaa ccagagaaca tcctgtttgt
gaattctgag tttgaaaccc tctacaatga gcacaagagc
tgtgaggaga agtcagtgaa gaacaccagc atccgagtgg
ctgactttgg cagtgccaca tttgaccatg agcaccacac
caccattgtg gccacccgtc actatcgccc gcctgaggtg
atccttgagc tgggctgggc acagccctgt gacgtctgga
gcattggctg cattctcttt gagtactacc ggggcttcac
actcttccag acccacgaaa accgagagca cctggtgatg
atggagaaga tcctagggcc catcccatca cacatgatcc
accgtaccag gaagcagaaa tatttctaca aagggggcct
agtttgggat gagaacagct ctgacggccg gtatgtgaag
gagaactgca aacctctgaa gagttacatg ctccaagact
ccctggagca cgtgcagctg tttgacctga tgaggaggat
gttagaattt gaccctgccc agcgcatcac actggccgag
gccctgctgc accccttctt tgctggcctg acccctgagg
agcggtcctt ccacaccagc cgcaacccaa gcagatga
Human CLK3 protein isoform 2
(SEQ ID NO: 15)
MHHCKRYRSPEPDPYLSYRWKRRRSYSREHEGRLRYPSRREPPP
RRSRSRSHDRLPYQRRYRERRDSDTYRCEERSPSFGEDYYGPSR
SRHRRRSRERGPYRTRKHAHHCHKRRTRSCSSASSRSQQSSKRS
SRSVEDDKEGHLVCRIGDWLQERYEIVGNLGEGTFGKVVECLDH
ARGKSQVALKIIRNVGKYREAARLEINVLKKIKEKDKENKFLCV
LMSDWFNFHGHMCIAFELLGKNTFEFLKENNFQPYPLPHVRHMA
YQLCHALRFLHENQLTHTDLKPENILFVNSEFETLYNEHKSCEE
KSVKNTSIRVADFGSATFDHEHHTTIVATRHYRPPEVILELGWA
QPCDVWSIGCILFEYYRGFTLFQTHENREHLVMMEKILGPIPSH
MIHRTRKQKYFYKGGLVWDENSSDGRYVKENCKPLKSYMLQDSL
EHVQLFDLMRRMLEFDPAQRITLAEALLHPFFAGLTPEERSFHT
SRNPSR
Human CLK3 cDNA isoform 2
(SEQ ID NO: 16)
atgca tcactgtaag cgataccgct cccctgaacc
agacccgtac ctgagctacc gatggaagag gaggaggtcc
tacagtcggg aacatgaagg gagactgcga tacccgtccc
gaagggagcc tcccccacga agatctcggt ccagaagcca
tgaccgcctg ccctaccaga ggaggtaccg ggagcgccgt
gacagcgata cataccggtg tgaagagcgg agcccatcct
ttggagagga ctactatgga ccttcacgtt ctcgtcatcg
tcggcgatcg cgggagaggg ggccataccg gacccgcaag
catgcccacc actgccacaa acgccgcacc aggtcttgta
gcagcgcctc ctcgagaagc caacagagca gtaagcgcag
cagccggagt gtggaagatg acaaggaggg tcacctggtg
tgccggatcg gcgattggct ccaagagcga tatgagattg
tggggaacct gggtgaaggc acctttggca aggtggtgga
gtgcttggac catgccagag ggaagtctca ggttgccctg
aagatcatcc gcaacgtggg caagtaccgg gaggctgccc
ggctagaaat caacgtgctc aaaaaaatca aggagaagga
caaagaaaac aagttcctgt gtgtcttgat gtctgactgg
ttcaacttcc acggtcacat gtgcatcgcc tttgagctcc
tgggcaagaa cacctttgag ttcctgaagg agaataactt
ccagccttac cccctaccac atgtccggca catggcctac
cagctctgcc acgcccttag atttctgcat gagaatcagc
tgacccatac agacttgaaa ccagagaaca tcctgtttgt
gaattctgag tttgaaaccc tctacaatga gcacaagagc
tgtgaggaga agtcagtgaa gaacaccagc atccgagtgg
ctgactttgg cagtgccaca tttgaccatg agcaccacac
caccattgtg gccacccgtc actatcgccc gcctgaggtg
atccttgagc tgggctgggc acagccctgt gacgtctgga
gcattggctg cattctcttt gagtactacc ggggcttcac
actcttccag acccacgaaa accgagagca cctggtgatg
atggagaaga tcctagggcc catcccatca cacatgatcc
accgtaccag gaagcagaaa tatttctaca aagggggcct
agtttgggat gagaacagct ctgacggccg gtatgtgaag
gagaactgca aacctctgaa gagttacatg ctccaagact
ccctggagca cgtgcagctg tttgacctga tgaggaggat
gttagaattt gaccctgccc agcgcatcac actggccgag
gccctgctgc accccttctt tgctggcctg acccctgagg
agcggtcctt ccacaccagc cgcaacccaa gcagatga
Human CLK4 protein
(SEQ ID NO: 17)
MRHSKRTHCPDWDSRESWGHESYRGSHKRKRRSHSSTQENRHCK
PHHQFKESDCHYLEARSLNERDYRDRRYVDEYRNDYCEGYVPRH
YHRDIESGYRIHCSKSSVRSRRS SPKRKRNRHCS SHQSRSKS
HRRKRSRSIEDDEEGHLICQ SGDVLRARYEIVDTLGEGAFGKV
VECIDHGMDGMHVAVKIVKNVGRYREAARSEIQVLEHLNSTDPN
SVFRCVQMLEWFDHHGHVCIVFELLGLSTYDFIKENSFLPFQID
HIRQMAYQICQSINFLHHNKLTHTDLKPENILFVKSDYVVKYNS
KMKRDERTLKNTDIKVVDFGSATYDDEHHSTLVSTRHYRAPEVI
LALGWSQPCDVWSIGCILIEYYLGFTVFQTHDSKEHLAMMERIL
GPIPQHMIQKTRKRKYFHHNQLDWDEHSSAGRYVRRRCKPLKEF
MLCHDEEHEKLFDLVRRMLEYDPTQRITLDEALQHPFFDLLKKK
Human CLK4 cDNA
(SEQ ID NO: 18)
at gcggcattcc aaaagaactc actgtcctga
ttgggatagc agagaaagct ggggacatga aagctatcgt
ggaagtcaca agcggaagag gagatctcat agtagcacac
aagagaacag gcattgtaaa ccacatcacc agtttaaaga
atctgattgt cattatttag aagcaaggtc cttgaatgag
cgagattatc gggaccggag atacgttgac gaatacagga
atgactactg tgaaggatat gttcctagac attatcacag
agacattgaa agcgggtatc gaatccactg cagtaaatct
tcagtccgca gcaggagaag cagtcctaaa aggaagcgca
atagacactg ttcaagtcat cagtcacgtt cgaagagcca
ccgaaggaaa agatccagga gtatagagga tgatgaggag
ggtcacctga tctgtcaaag tggagacgtt ctaagagcaa
gatatgaaat cgtggacact ttgggtgaag gagcctttgg
caaagttgta gagtgcattg atcatggcat ggatggcatg
catgtagcag tgaaaatcgt aaaaaatgta ggccgttacc
gtgaagcagc tcgttcagaa atccaagtat tagagcactt
aaatagtact gatcccaata gtgtcttccg atgtgtccag
atgctagaat ggtttgatca tcatggtcat gtttgtattg
tgtttgaact actgggactt agtacttacg atttcattaa
agaaaacagc tttctgccat ttcaaattga ccacatcagg
cagatggcgt atcagatctg ccagtcaata aattttttac
atcataataa attaacccat acagatctga agcctgaaaa
tattttgttt gtgaagtctg actatgtagt caaatataat
tctaaaatga aacgtgatga acgcacactg aaaaacacag
atatcaaagt tgttgacttt ggaagtgcaa cgtatgatga
tgaacatcac agtactttgg tgtctacccg gcactacaga
gctcccgagg tcattttggc tttaggttgg tctcagcctt
gtgatgtttg gagcataggt tgcattctta ttgaatatta
ccttggtttc acagtctttc agactcatga tagtaaagag
cacctggcaa tgatggaacg aatattagga cccataccac
aacacatgat tcagaaaaca agaaaacgca agtattttca
ccataaccag ctagattggg atgaacacag ttctgctggt
agatatgtta ggagacgctg caaaccgttg aaggaattta
tgctttgtca tgatgaagaa catgagaaac tgtttgacct
ggttcgaaga atgttagaat atgatccaac tcaaagaatt
accttggatg aagcattgca gcatcctttc tttgacttat
taaaaaagaa atga

### Exemplary CLK Inhibitors

In some embodiments, the CLK inhibitor inhibits one or more of the CLK family members CLK1, CLK2, CLK3, and CLK4. In some embodiments, the CLK inhibitor is a broad spectrum CLK inhibitor, inhibiting two or more CLK family members CLK1, CLK2, CLK3, and CLK4. In some embodiments, the CLK inhibitor inhibits CLK2, CLK3, or both CLK2 and CLK3. In some embodiments, the CLK inhibitor has an IC₅₀ of between about 100 pM and about 10 μM (e.g., between about 100 pM and about 9 μM, between about 100 pM and about 8 μM, between about 100 pM and about 7 μM, between about 100 pM and about 6 μM, between about 100 pM and about 5 μM, between about 100 pM and about 4 μM, between about 100 pM and about 3 μM, between about 100 pM and about 2 μM, between about 100 pM and about 1 μM, between about 100 pM and about 950 nM, between about 100 pM and about 900 nM, between about 100 pM and about 850 nM, between about 100 pM and about 800 nM, between about 100 pM and about 750 nM, between about 100 pM and about 700 nM, between about 100 pM and about 650 nM, between about 100 pM and about 600 nM, between about 100 pM and about 550 nM, between about 100 pM and about 500 nM, between about 100 pM and about 450 nM, between about 100 pM and about 400 nM, between about 100 pM and about 350 nM, between about 100 pM and about 300 nM, between about 100 pM and about 250 nM, between about 100 pM and about 200 nM, between about 100 pM and about 150 nM, between about 100 pM and about 100 nM, between about 100 pM and about 95 nM, between about 100 pM and about 90 nM, between about 100 pM and about 85 nM, between about 100 pM and about 80 nM, between about 100 pM and about 75 nM, between about 100 pM and about 70 nM, between about 100 pM and about 65 nM, between about 100 pM and about 60 nM, between about 100 pM and about 55 nM, between about 100 pM and about 50 nM, between about 100 pM and about 45 nM, between about 100 pM and about 40 nM, between about 100 pM and about 35 nM, between about 100 pM and about 30 nM, between about 100 pM and about 25 nM, between about 100 pM and about 20 nM, between about 100 pM and about 15 nM, between about 100 pM and about 10 nM, between about 100 pM and about 5 nM, between about 100 pM and about 4 nM, between about 100 pM and about 3 nM, between about 100 pM and about 2 nM, e.g., between about 1 nM and about 9 μM, between about 1 nM and about 8 μM, between about 1 nM and about 7 μM, between about 1 nM and about 6 μM, between about 1 nM and about 5 μM, between about 1 nM and about 4 μM, between about 1 nM and about 3 μM, between about 1 nM and about 2 μM, between about 1 nM and about 1 μM, between about 1 nM and about 950 nM, between about 1 nM and about 900 nM, between about 1 nM and about 850 nM, between about 1 nM and about 800 nM, between about 1 nM and about 750 nM, between about 1 nM and about 700 nM, between about 1 nM and about 650 nM, between about 1 nM and about 600 nM, between about 1 nM and about 550 nM, between about 1 nM and about 500 nM, between about 1 nM and about 450 nM, between about 1 nM and about 400 nM, between about 1 nM and about 350 nM, between about 1 nM and about 300 nM, between about 1 nM and about 250 nM, between about 1 nM and about 200 nM, between about 1 nM and about 150 nM, between about 1 nM and about 100 nM, between about 1 nM and about 95 nM, between about 1 nM and about 90 nM, between about 1 nM and about 85 nM, between about 1 nM and about 80 nM, between about 1 nM and about 75 nM, between about 1 nM and about 70 nM, between about 1 nM and about 65 nM, between about 1 nM and about 60 nM, between about 1 nM and about 55 nM, between about 1 nM and about 50 nM, between about 1 nM and about 45 nM, between about 1 nM and about 40 nM, between about 1 nM and about 35 nM, between about 1 nM and about 30 nM, between about 1 nM and about 25 nM, between about 1 nM and about 20 nM, between about 1 nM and about 15 nM, between about 1 nM and about 10 nM, between about 1 nM and about 5 nM, between about 1 nM and about 4 nM, between about 1 nM and about 3 nM, between about 1 nM and about 2 nM, between about 2 nM and about 10 μM, between about 2 nM and about 9 μM, between about 2 nM and about 8 μM, between about 2 nM and about 7 μM, between about 2 nM and about 6 μM, between about 2 nM and about 5 μM, between about 2 nM and about 4 μM, between about 2 nM and about 3 μM, between about 2 nM and about 2 μM, between about 2 nM and about 1 μM, between about 2 nM and about 950 nM, between about 2 nM and about 900 nM, between about 2 nM and about 850 nM, between about 2 nM and about 800 nM, between about 2 nM and about 750 nM, between about 2 nM and about 700 nM, between about 2 nM and about 650 nM, between about 2 nM and about 600 nM, between about 2 nM and about 550 nM, between about 2 nM and about 500 nM, between about 2 nM and about 450 nM, between about 2 nM and about 400 nM, between about 2 nM and about 350 nM, between about 2 nM and about 300 nM, between about 2 nM and about 250 nM, between about 2 nM and about 200 nM, between about 2 nM and about 150 nM, between about 2 nM and about 100 nM, between about 2 nM and about 95 nM, between about 2 nM and about 90 nM, between about 2 nM and about 85 nM, between about 2 nM and about 80 nM, between about 2 nM and about 75 nM, between about 2 nM and about 70 nM, between about 2 nM and about 65 nM, between about 2 nM and about 60 nM, between about 2 nM and about 55 nM, between about 2 nM and about 50 nM, between about 2 nM and about 45 nM, between about 2 nM and about 40 nM, between about 2 nM and about 35 nM, between about 2 nM and about 30 nM, between about 2 nM and about 25 nM, between about 2 nM and about 20 nM, between about 2 nM and about 15 nM, between about 2 nM and about 10 nM, between about 2 nM and about 5 nM, between about 2 nM and about 4 nM, between about 2 nM and about 3 nM, between about 5 nM and about 10 μM, between about 5 nM and about 9 μM, between about 5 nM and about 8 μM, between about 5 nM and about 7 μM, between about 5 nM and about 6 μM, between about 5 nM and about 5 μM, between about 5 nM and about 4 μM, between about 5 nM and about 3 μM, between about 5 nM and about 2 μM, between about 5 nM and about 1 μM, between about 5 nM and about 950 nM, between about 5 nM and about 900 nM, between about 5 nM and about 850 nM, between about 5 nM and about 800 nM, between about 5 nM and about 750 nM, between about 5 nM and about 700 nM, between about 5 nM and about 650 nM, between about 5 nM and about 600 nM, between about 5 nM and about 550 nM, between about 5 nM and about 500 nM, between about 5 nM and about 450 nM, between about 5 nM and about 400 nM, between about 5 nM and about 350 nM, between about 5 nM and about 300 nM, between about 5 nM and about 250 nM, between about 5 nM and about 200 nM, between about 5 nM and about 150 nM, between about 5 nM and about 100 nM, between about 5 nM and about 95 nM, between about 5 nM and about 90 nM, between about 5 nM and about 85 nM, between about 5 nM and about 80 nM, between about 5 nM and about 75 nM, between about 5 nM and about 70 nM, between about 5 nM and about 65 nM, between about 5 nM and about 60 nM, between about 5 nM and about 55 nM, between about 5 nM and about 50 nM, between about 5 nM and about 45 nM, between about 5 nM and about 40 nM, between about 5 nM and about 35 nM, between about 5 nM and about 30 nM, between about 5 nM and about 25 nM, between about 5 nM and about 20 nM, between about 5 nM and about 15 nM, between about 5 nM and about 10 nM, between about 10 nM and about 10 μM, between about 10 nM and about 9 μM, between about 10 nM and about 8 μM, between about 10 nM and about 7 μM, between about 10 nM and about 6 μM, between about 10 nM and about 5 μM, between about 10 nM and about 4 μM, between about 10 nM and about 3 μM, between about 10 nM and about 2 μM, between about 10 nM and about 1 μM, between about 10 nM and about 950 nM, between about 10 nM and about 900 nM, between about 10 nM and about 850 nM, between about 10 nM and about 800 nM, between about 10 nM and about 750 nM, between about 10 nM and about 700 nM, between about 10 nM and about 650 nM, between about 10 nM and about 600 nM, between about 10 nM and about 550 nM, between about 10 nM and about 500 nM, between about 10 nM and about 450 nM, between about 10 nM and about 400 nM, between about 10 nM and about 350 nM, between about 10 nM and about 300 nM, between about 10 nM and about 250 nM, between about 10 nM and about 200 nM, between about 10 nM and about 150 nM, between about 10 nM and about 100 nM, between about 10 nM and about 95 nM, between about 10 nM and about 90 nM, between about 10 nM and about 85 nM, between about 10 nM and about 80 nM, between about 10 nM and about 75 nM, between about 10 nM and about 70 nM, between about 10 nM and about 65 nM, between about 10 nM and about 60 nM, between about 10 nM and about 55 nM, between about 10 nM and about 50 nM, between about 10 nM and about 45 nM, between about 10 nM and about 40 nM, between about 10 nM and about 35 nM, between about 10 nM and about 30 nM, between about 10 nM and about 25 nM, between about 10 nM and about 20 nM, between about 10 nM and about 15 nM, between about 50 nM and about 10 μM, between about 50 nM and about 9 μM, between about 50 nM and about 8 μM, between about 50 nM and about 7 μM, between about 50 nM and about 6 μM, between about 50 nM and about 5 μM, between about 50 nM and about 4 μM, between about 50 nM and about 3 μM, between about 50 nM and about 2 μM, between about 50 nM and about 1 μM, between about 50 nM and about 950 nM, between about 50 nM and about 900 nM, between about 50 nM and about 850 nM, between about 50 nM and about 800 nM, between about 50 nM and about 750 nM, between about 50 nM and about 700 nM, between about 50 nM and about 650 nM, between about 50 nM and about 600 nM, between about 50 nM and about 550 nM, between about 50 nM and about 500 nM, between about 50 nM and about 450 nM, between about 50 nM and about 400 nM, between about 50 nM and about 350 nM, between about 50 nM and about 300 nM, between about 50 nM and about 250 nM, between about 50 nM and about 200 nM, between about 50 nM and about 150 nM, between about 50 nM and about 100 nM, between about 50 nM and about 95 nM, between about 50 nM and about 90 nM, between about 50 nM and about 85 nM, between about 50 nM and about 80 nM, between about 50 nM and about 75 nM, between about 50 nM and about 70 nM, between about 50 nM and about 65 nM, between about 50 nM and about 60 nM, between about 50 nM and about 55 nM, between about 100 nM and about 10 μM, between about 100 nM and about 9 μM, between about 100 nM and about 8 μM, between about 100 nM and about 7 μM, between about 100 nM and about 6 μM, between about 100 nM and about 5 μM, between about 100 nM and about 4 μM, between about 100 nM and about 3 μM, between about 100 nM and about 2 μM, between about 100 nM and about 1 μM, between about 100 nM and about 950 nM, between about 100 nM and about 900 nM, between about 100 nM and about 850 nM, between about 100 nM and about 800 nM, between about 100 nM and about 750 nM, between about 100 nM and about 700 nM, between about 100 nM and about 650 nM, between about 100 nM and about 600 nM, between about 100 nM and about 550 nM, between about 100 nM and about 500 nM, between about 100 nM and about 450 nM, between about 100 nM and about 400 nM, between about 100 nM and about 350 nM, between about 100 nM and about 300 nM, between about 100 nM and about 250 nM, between about 100 nM and about 200 nM, between about 100 nM and about 150 nM, between about 200 nM and about 10 μM, between about 200 nM and about 9 μM, between about 200 nM and about 8 μM, between about 200 nM and about 7 μM, between about 200 nM and about 6 μM, between about 200 nM and about 5 μM, between about 200 nM and about 4 μM, between about 200 nM and about 3 μM, between about 200 nM and about 2 μM, between about 200 nM and about 1 μM, between about 200 nM and about 950 nM, between about 200 nM and about 900 nM, between about 200 nM and about 850 nM, between about 200 nM and about 800 nM, between about 200 nM and about 750 nM, between about 200 nM and about 700 nM, between about 200 nM and about 650 nM, between about 200 nM and about 600 nM, between about 200 nM and about 550 nM, between about 200 nM and about 500 nM, between about 200 nM and about 450 nM, between about 200 nM and about 400 nM, between about 200 nM and about 350 nM, between about 200 nM and about 300 nM, between about 200 nM and about 250 nM, between about 250 nM and about 10 μM, between about 250 nM and about 9 μM, between about 250 nM and about 8 μM, between about 250 nM and about 7 μM, between about 250 nM and about 6 μM, between about 250 nM and about 5 μM, between about 250 nM and about 4 μM, between about 250 nM and about 3 μM, between about 250 nM and about 2 μM, between about 250 nM and about 1 μM, between about 250 nM and about 950 nM, between about 250 nM and about 900 nM, between about 250 nM and about 850 nM, between about 250 nM and about 800 nM, between about 250 nM and about 750 nM, between about 250 nM and about 700 nM, between about 250 nM and about 650 nM, between about 250 nM and about 600 nM, between about 250 nM and about 550 nM, between about 250 nM and about 500 nM, between about 250 nM and about 450 nM, between about 250 nM and about 400 nM, between about 250 nM and about 350 nM, between about 250 nM and about 300 nM, between about 500 nM and about 10 μM, between about 500 nM and about 9 μM, between about 500 nM and about 8 μM, between about 500 nM and about 7 μM, between about 500 nM and about 6 μM, between about 500 nM and about 5 μM, between about 500 nM and about 4 μM, between about 500 nM and about 3 μM, between about 500 nM and about 2 μM, between about 500 nM and about 1 μM, between about 500 nM and about 950 nM, between about 500 nM and about 900 nM, between about 500 nM and about 850 nM, between about 500 nM and about 800 nM, between about 500 nM and about 750 nM, between about 500 nM and about 700 nM, between about 500 nM and about 650 nM, between about 500 nM and about 600 nM, between about 500 nM and about 550 nM, between about 750 nM and about 10 μM, between about 750 nM and about 9 μM, between about 750 nM and about 8 μM, between about 750 nM and about 7 μM, between about 750 nM and about 6 μM, between about 750 nM and about 5 μM, between about 750 nM and about 4 μM, between about 750 nM and about 3 μM, between about 750 nM and about 2 μM, between about 750 nM and about 1 μM, between about 750 nM and about 950 nM, between about 750 nM and about 900 nM, between about 750 nM and about 850 nM, between about 750 nM and about 800 nM, between about 950 nM and about 10 μM, between about 950 nM and about 9 μM, between about 950 nM and about 8 μM, between about 950 nM and about 7 μM, between about 950 nM and about 6 μM, between about 950 nM and about 5 μM, between about 950 nM and about 4 μM, between about 950 nM and about 3 μM, between about 950 nM and about 2 μM, between about 950 nM and about 1 μM, between about 1 μM and about 10 μM, between about 1 μM and about 9 μM, between about 1 μM and about 8 μM, between about 1 μM and about 7 μM, between about 1 μM and about 6 μM, between about 1 μM and about 5 μM, between about 1 μM and about 4 μM, between about 1 μM and about 3 μM, between about 1 μM and about 2 μM, between about 2 μM and about 10 μM, between about 2 μM and about 9 μM, between bout 2 μM and about 8 μM, between about 2 μM and about 7 μM, between about 2 μM and about 6 μM, between about 2 μM and about 5 μM, between about 2 μM and about 4 μM, between about 2 μM and about 3 μM, between about 4 μM and about 10 μM, between about 4 μM and about 9 μM, between about 4 μM and about 8 μM, between about 4 μM and about 7 μM, between about 4 μM and about 6 μM, between about 4 μM and about 5 μM, between about 5 μM and about 10 μM, between about 5 μM and about 9 μM, between about 5 μM and about 8 μM, between about 5 μM and about 7 μM, between about 5 μM and about 6 μM, between about 6 μM and about 10 μM, between about 6 μM and about 9 μM, between about 6 μM and about 8 μM, between about 6 μM and about 7 μM; between about 7 μM and about 10 μM, between about 7 μM and about 9 μM, between about 7 μM and about 8 μM, between about 8 μM and about 10 μM, between about 8 μM and about 9 μM, or between about 9 μM and about 10 μM) for one or more of CLK1, CLK2, CLK3, and CLK4.

In some embodiments, the CLK inhibitor has an IC₅₀ of between about 100 pM and about 10 μM (or any of the subranges of this range described herein) for each of CLK3 and CLK4. In some embodiments, the CLK inhibitor has an IC₅₀ of between about 100 pM and about 10 μM (or any of the subranges of this range) for each of CLK1 and CLK3. In some embodiments, the CLK inhibitor has an IC₅₀ of between about 100 pM and about 10 μM (or any of the subranges of this range described herein) for each of CLK1 and CLK2. In some embodiments, the CLK inhibitor has an IC₅₀ of between about 100 pM and about 10 μM (or any of the subranges of this range described herein) for each of CLK1 and CLK4. In some embodiments, the CLK inhibitor has an IC₅₀ of between about 100 pM and about 10 μM (or any of the subranges of this range described herein) for each of CLK2 and CLK4. In some embodiments, the CLK inhibitor has an IC₅₀ of between about 100 pM and about 10 μM (or any of the subranges of this range described herein) for each of CLK1, CLK2, and/or CLK3. In some embodiments, the CLK inhibitor has an IC₅₀ of between about 100 pM and about 10 μM (or any of the subranges of this range described herein) for each of CLK1, CLK2 and CLK4. In some embodiments, the CLK inhibitor has an IC₅₀ of between about 100 pM and about 10 μM (or any of the subranges of this range described herein) for each of CLK2, CLK3 and CLK4. In some embodiments, the CLK inhibitor has an IC₅₀ of between about 100 pM and about 10 μM (or any of the subranges of this range described herein) for each of CLK1, CLK2, CLK3 and CLK4.

In some embodiments, the CLK inhibitor is a compound of Formula (I), or a pharmaceutically acceptable salt or solvate thereof.

In some embodiments, the CLK inhibitor is a compound of Formula (II), or a pharmaceutically acceptable salt or solvate thereof.

In some embodiments, the CLK inhibitor is a compound of Formula (III), or a pharmaceutically acceptable salt or solvate thereof.

In some embodiments, the CLK inhibitor is a compound of Formula (IV), or a pharmaceutically acceptable salt or solvate thereof.

In some embodiments, the CLK inhibitor is a compound of Formula (V), or a pharmaceutically acceptable salt or solvate thereof.

In some embodiments, the CLK inhibitor is a compound of Formula (VI), or a pharmaceutically acceptable salt or solvate thereof.

In some embodiments, the CLK inhibitor is a compound of Formula (VII), or a pharmaceutically acceptable salt or solvate thereof.

In some embodiments, the CLK inhibitor is a compound of Formula (VIII), or a pharmaceutically acceptable salt or solvate thereof.

### DYRK Family

The DYRK (dual-specificity tyrosine phosphorylation-regulated kinases) family of kinases contains five members (DYRK1A, DYRK1B, DYRK2, DYRK3, and DYRK4). DYRKs are proposed to function by directly phosphorylating serine and threonine residues on target proteins, such as, but not limited to: STAT3, Gli1, JNK1, Sirt1, Foxo1/3, dynamin, glycogen synthase, CREB, tau, and Hip-1. They have been proposed to be implicated in, but not limited to: cell survival, proliferation and differentiation, and in the pathology of Down Syndrome, Alzheimer's Disease, Parkinson's Disease, and Huntington's Disease.

Exemplary human DYRK1A protein sequences are SEQ ID NO: 19, 21, 23, 25, and 27. Exemplary human cDNA sequences that encode DYRK1A are SEQ ID NO: 20, 22, 24, 26, and 28. Exemplary human DYRK1B protein sequences are SEQ ID NO: 29, 31, and 33. Exemplary human cDNA sequences that encode DYRK1B are SEQ ID NO: 30, 32, and 34. Exemplary human DYRK2 protein sequences are SEQ ID NO: 35 and 37. Exemplary human cDNA sequences that encode DYRK2 are SEQ ID NO: 36 and 38. Exemplary human DYRK3 protein sequences are SEQ ID NO: 39 and 41. Exemplary human cDNA sequences that encode DYRK3 are SEQ ID NO: 40 and 42. Exemplary human DYRK4 protein sequences are SEQ ID NO: 43, 45, 47, 48, and 49. Exemplary human cDNA sequences that encode DYRK4 are SEQ ID NO: 44 and 46.

Human DYRKIA protein long isoform
(SEQ ID NO: 19)
MHTGGETSACKPSSVRLAPSFSFHAAGLQMAGQMPHSHQYSDRRQ
PNISDQQVSALSYSDQIQQPLTNQVMPDIVMLQRRMPQTFRDPA
TAPLRKLSVDLIKTYKHINEVYYAKKKRRHQQGQGDDSSHKKERK
VYNDGYDDDNYDYIVKNGEKWMDRYEIDSLIGKGSFGQVVKAYDR
VEQEWVAIKIIKNKKAFLNQAQIEVRLLELMNKHDTEMKYYIVHL
KRHFMFRNHLCLVFEMLSYNLYDLLRNTNFRGVSLNLTRKFAQQM
CTALLFLATPELSIIHCDLKPENILLCNPKRSAIKIVDFGSSCQL
GQRIYQYIQSRFYRSPEVLLGMPYDLAIDMWSLGCILVEMHTGEP
LFSGANEVDQMNKIVEVLGIPPAHILDQAPKARKFFEKLPDGTWN
LKKTKDGKREYKPPGTRKLHNILGVETGGPGGRRAGESGHTVADY
LKFKDLILRMLDYDPKTRIQPYYALQHSFFKKTADEGTNTSNSVS
TSPAMEQSQSSGTTSSTSSSSGGSSGTSNSGRARSDPTHQHRHSG
GHFTAAVQAMDCETHSPQVRQQFPAPLGWSGTEAPTQVTVETHPV
QETTFHVAPQQNALHHHHGNSSHHHHHHHHHHHHHGQQALGNRTR
PRVYNSPTNSSSTQDSMEVGHSHHSMTSLSSSTTSSSTSSSSTGN
QGNQAYQNRPVAANTLDFGQNGAMDVNLTVYSNPRQETGIAGHPT
YQFSANTGPAHYMTEGHLTMRQGADREESPMTGVCVQQSPVASS
DYRKIA cDNA long isoform
(SEQ ID NO: 20)
atgcatacaggaggagagacttcagcatgcaaaccttcatctgtt
cggcttgcaccgtcattttcattccatgctgctggccttcagat
ggctggacagatgccccattcacatcagtacagtgaccgtcgcca
gccaaacataagtgaccaacaggtttctgccttatcatattctga
ccagattcagcaacctctaactaaccaggtgatgcctgatattgt
catgttacagaggcggatgccccaaaccttccgtgacccagcaac
tgctcccctgagaaaactttctgttgacttgatcaaaacatacaa
gcatattaatgaggtttactatgcaaaaaagaagcgaagacacca
acagggccagggagacgattctagtcataagaaggaacggaaggt
ttacaatgatggttatgatgatgataactatgattatattgtaaa
aaacggagaaaagtggatggatcgttacgaaattgactccttgat
aggcaaaggttcctttggacaggttgtaaaggcatatgatcgtgt
ggagcaagaatgggttgccattaaaataataaagaacaagaaggc
ttttctgaatcaagcacagatagaagtgcgacttcttgagctcat
gaacaaacatgacactgaaatgaaatactacatagtgcatttgaa
acgccactttatgtttcgaaaccatctctgtttagtttttgaaat
gctgtcctacaacctctatgacttgctgagaaacaccaatttccg
aggggtctctttgaacctaacacgaaagtttgcgcaacagatgtg
cactgcactgcttttccttgcgactccagaacttagtatcattca
ctgtgatctaaaacctgaaaatatccttctttgtaaccccaaacg
cagtgcaatcaagatagttgactttggcagttcttgtcagttggg
gcagaggatataccagtatattcagagtcgcttttatcggtctcc
agaggtgctactgggaatgccttatgaccttgccattgatatgtg
gtccctcgggtgtattttggttgaaatgcacactggagaacctct
gttcagtggtgccaatgaggtagatcagatgaataaaatagtgga
agttctgggtattccacctgctcatattcttgaccaagcaccaaa
agcaagaaagttctttgagaagttgccagatggcacttggaactt
aaagaagaccaaagatggaaaacgggagtacaaaccaccaggaac
ccgtaaacttcataacattcttggagtggaaacaggaggacctgg
tgggcgacgtgctggggagtcaggtcatacggtcgctgactactt
gaagttcaaagacctcattttaaggatgcttgattatgaccccaa
aactcgaattcaaccttattatgctctgcagcacagtttcttcaa
gaaaacagctgatgaaggtacaaatacaagtaatagtgtatctac
aagccccgccatggagcagtctcagtcttcgggcaccacctccag
tacatcgtcaagctcaggtggctcatcggggacaagcaacagtgg
gagagcccggtcggatccgacgcaccagcatcggcacagtggtgg
gcacttcacagctgccgtgcaggccatggactgcgagacacacag
tccccaggtgcgtcagcaatttcctgctcctcttggttggtcagg
cactgaagctcctacacaggtcactgttgaaactcatcctgttca
agaaacaacctttcatgtagcccctcaacagaatgcattgcatca
tcaccatggtaacagttcccatcaccatcaccaccaccaccacca
tcaccaccaccatggacaacaagccttgggtaaccggaccaggcc
aagggtctacaattctccaacgaatagctcctctacccaagattc
tatggaggttggccacagtcaccactccatgacatccctgtcttc
ctcaacgacttcttcctcgacatcttcctcctctactggtaacca
aggcaatcaggcctaccagaatcgcccagtggctgctaatacctt
ggactttggacagaatggagctatggacgttaatttgaccgtcta
ctccaatccccgccaagagactggcatagctggacatccaacata
ccaattttctgctaatacaggtcctgcacattacatgactgaagg
acatctgacaatgaggcaaggggctgatagagaagagtcccccat
gacaggagtttgtgtgcaacagagtcctgtagctagctcgtga
DYRKIA protein isoform 1
(SEQ ID NO: 21)
MHTGGETSACKPSSVRLAPSFSFHAAGLQMAGQMPHSHQYSDRRQ
PNISDQQVSALSYSDQIQQPLTNQRRMPQTFRDPATAPLRKLSV
DLIKTYKHINEVYYAKKKRRHQQGQGDDSSHKKERKVYNDGYDDD
NYDYIVKNGEKWMDRYEIDSLIGKGSFGQVVKAYDRVEQEWVAIK
IIKNKKAFLNQAQIEVRLLELMNKHDTEMKYYIVHLKRHFMFRNH
LCLVFEMLSYNLYDLLRNTNFRGVSLNLTRKFAQQMCTALLFLAT
PELSIIHCDLKPENILLCNPKRSAIKIVDFGSSCQLGQRIYQYIQ
SRFYRSPEVLLGMPYDLAIDMWSLGCILVEMHTGEPLFSGANEVD
QMNKIVEVLGIPPAHILDQAPKARKFFEKLPDGTWNLKKTKDGKR
EYKPPGTRKLHNILGVETGGPGGRRAGESGHTVADYLKFKDLILR
MLDYDPKTRIQPYYALQHSFFKKTADEGTNTSNSVSTSPAMEQSQ
SSGTTSSTSSSSGGSSGTSNSGRARSDPTHQHRHSGGHFTAAVQA
MDCETHSPQVRQQFPAPLGWSGTEAPTQVTVETHPVQETTFHVAP
QQNALHHHHGNSSHHHHHHHHHHHHHGQQALGNRTRPRVYNSPTN
SSSTQDSMEVGHSHHSMTSLSSSTTSSSTSSSSTGNQGNQAYQNR
PVAANTLDFGQNGAMDVNLTVYSNPRQETGIAGHPTYQFSANTGP
AHYMTEGHLTMRQGADREESPMTGVCVQQSPVASS
DYRKIA cDNA isoform 1
(SEQ ID NO: 22)
atgcatacaggaggagagacttcagcatgcaaaccttcatctgtt
cggcttgcaccgtcattttcattccatgctgctggccttcagat
ggctggacagatgccccattcacatcagtacagtgaccgtcgcca
gccaaacataagtgaccaacaggtttctgccttatcatattctga
ccagattcagcaacctctaactaaccagaggcggatgccccaaac
cttccgtgacccagcaactgctcccctgagaaaactttctgttga
cttgatcaaaacatacaagcatattaatgaggtttactatgcaaa
aaagaagcgaagacaccaacagggccagggagacgattctagtca
taagaaggaacggaaggtttacaatgatggttatgatgatgataa
ctatgattatattgtaaaaaacggagaaaagtggatggatcgtta
cgaaattgactccttgataggcaaaggttcctttggacaggttgt
aaaggcatatgatcgtgtggagcaagaatgggttgccattaaaat
aataaagaacaagaaggcttttctgaatcaagcacagatagaagt
gcgacttcttgagctcatgaacaaacatgacactgaaatgaaata
ctacatagtgcatttgaaacgccactttatgtttcgaaaccatct
ctgtttagtttttgaaatgctgtcctacaacctctatgacttgct
gagaaacaccaatttccgaggggtctctttgaacctaacacgaaa
gtttgcgcaacagatgtgcactgcactgcttttccttgcgactcc
agaacttagtatcattcactgtgatctaaaacctgaaaatatcct
tctttgtaaccccaaacgcagtgcaatcaagatagttgactttgg
cagttcttgtcagttggggcagaggatataccagtatattcagag
tcgcttttatcggtctccagaggtgctactgggaatgccttatga
ccttgccattgatatgtggtccctcgggtgtattttggttgaaat
gcacactggagaacctctgttcagtggtgccaatgaggtagatca
gatgaataaaatagtggaagttctgggtattccacctgctcatat
tcttgaccaagcaccaaaagcaagaaagttctttgagaagttgcc
agatggcacttggaacttaaagaagaccaaagatggaaaacggga
gtacaaaccaccaggaacccgtaaacttcataacattcttggagt
ggaaacaggaggacctggtgggcgacgtgctggggagtcaggtca
tacggtcgctgactacttgaagttcaaagacctcattttaaggat
gcttgattatgaccccaaaactcgaattcaaccttattatgctct
gcagcacagtttcttcaagaaaacagctgatgaaggtacaaatac
aagtaatagtgtatctacaagccccgccatggagcagtctcagtc
ttcgggcaccacctccagtacatcgtcaagctcaggtggctcatc
ggggacaagcaacagtgggagagcccggtcggatccgacgcacca
gcatcggcacagtggtgggcacttcacagctgccgtgcaggccat
ggactgcgagacacacagtccccaggtgcgtcagcaatttcctgc
tcctcttggttggtcaggcactgaagctcctacacaggtcactgt
tgaaactcatcctgttcaagaaacaacctttcatgtagcccctca
acagaatgcattgcatcatcaccatggtaacagttcccatcacca
tcaccaccaccaccaccatcaccaccaccatggacaacaagcctt
gggtaaccggaccaggccaagggtctacaattctccaacgaatag
ctcctctacccaagattctatggaggttggccacagtcaccactc
catgacatccctgtcttcctcaacgacttcttcctcgacatcttc
ctcctctactggtaaccaaggcaatcaggcctaccagaatcgccc
agtggctgctaataccttggactttggacagaatggagctatgga
cgttaatttgaccgtctactccaatccccgccaagagactggcat
agctggacatccaacataccaattttctgctaatacaggtcctgc
acattacatgactgaaggacatctgacaatgaggcaaggggctga
tagagaagagtcccccatgacaggagtttgtgtgcaacagagtcc
tgtagctagctcgtga
DYRKIA protein isoform 2
(SEQ ID NO: 23)
MHTGGETSACKPSSVRLAPSFSFHAAGLQMAGQMPHSHQYSDRRQ
PNISDQQVSALSYSDQIQQPLTNQVMPDIVMLQRRMPQTFRDPA
TAPLRKLSVDLIKTYKHINEVYYAKKKRRHQQGQGDDSSHKKERK
VYNDGYDDDNYDYIVKNGEKWMDRYEIDSLIGKGSFGQVVKAYDR
VEQEWVAIKIIKNKKAFLNQAQIEVRLLELMNKHDTEMKYYIVHL
KRHFMFRNHLCLVFEMLSYNLYDLLRNTNFRGVSLNLTRKFAQQM
CTALLFLATPELSIIHCDLKPENILLCNPKRSAIKIVDFGSSCQL
GQRIYQYIQSRFYRSPEVLLGMPYDLAIDMWSLGCILVEMHTGEP
LFSGANEVDQMNKIVEVLGIPPAHILDQAPKARKFFEKLPDGTWN
LKKTKDGKREYKPPGTRKLHNILGVETGGPGGRRAGESGHTVADY
LKFKDLILRMLDYDPKTRIQPYYALQHSFFKKTADEGTNTSNSVS
TSPAMEQSQSSGTTSSTSSSSGASAISCSSWLVRH
DYRKIA cDNA isoform 2
(SEQ ID NO: 24)
atgcatacaggaggagagacttcagcatgcaaaccttcatctgtt
cggcttgcaccgtcattttcattccatgctgctggccttcagat
ggctggacagatgccccattcacatcagtacagtgaccgtcgcca
gccaaacataagtgaccaacaggtttctgccttatcatattctga
ccagattcagcaacctctaactaaccaggtgatgcctgatattgt
catgttacagaggcggatgccccaaaccttccgtgacccagcaac
tgctcccctgagaaaactttctgttgacttgatcaaaacatacaa
gcatattaatgaggtttactatgcaaaaaagaagcgaagacacca
acagggccagggagacgattctagtcataagaaggaacggaaggt
ttacaatgatggttatgatgatgataactatgattatattgtaaa
aaacggagaaaagtggatggatcgttacgaaattgactccttgat
aggcaaaggttcctttggacaggttgtaaaggcatatgatcgtgt
ggagcaagaatgggttgccattaaaataataaagaacaagaaggc
ttttctgaatcaagcacagatagaagtgcgacttcttgagctcat
gaacaaacatgacactgaaatgaaatactacatagtgcatttgaa
acgccactttatgtttcgaaaccatctctgtttagtttttgaaat
gctgtcctacaacctctatgacttgctgagaaacaccaatttccg
aggggtctctttgaacctaacacgaaagtttgcgcaacagatgtg
cactgcactgcttttccttgcgactccagaacttagtatcattca
ctgtgatctaaaacctgaaaatatccttctttgtaaccccaaacg
cagtgcaatcaagatagttgactttggcagttcttgtcagttggg
gcagaggatataccagtatattcagagtcgcttttatcggtctcc
agaggtgctactgggaatgccttatgaccttgccattgatatgtg
gtccctcgggtgtattttggttgaaatgcacactggagaacctct
gttcagtggtgccaatgaggtagatcagatgaataaaatagtgga
agttctgggtattccacctgctcatattcttgaccaagcaccaaa
agcaagaaagttctttgagaagttgccagatggcacttggaactt
aaagaagaccaaagatggaaaacgggagtacaaaccaccaggaac
ccgtaaacttcataacattcttggagtggaaacaggaggacctgg
tgggcgacgtgctggggagtcaggtcatacggtcgctgactactt
gaagttcaaagacctcattttaaggatgcttgattatgaccccaa
aactcgaattcaaccttattatgctctgcagcacagtttcttcaa
gaaaacagctgatgaaggtacaaatacaagtaatagtgtatctac
aagccccgccatggagcagtctcagtcttcgggcaccacctccag
tacatcgtcaagctcaggtgcgtcagcaatttcctgctcctcttg
gttggtcaggcactga
DYRKIA protein isoform 3
(SEQ ID NO: 25)
MHTGGETSACKPSSVRLAPSFSFHAAGLQMAGQMPHSHQYSDRRQ
PNISDQQVSALSYSDQIQQPLTNQVMPDIVMLQRRMPQTFRDPA
TAPLRKLSVDLIKTYKHINEVYYAKKKRRHQQGQGDDSSHKKERK
VYNDGYDDDNYDYIVKNGEKWMDRYEIDSLIGKGSFGQVVKAYDR
VEQEWVAIKIIKNKKAFLNQAQIEVRLLELMNKHDTEMKYYIVHL
KRHFMFRNHLCLVFEMLSYNLYDLLRNTNFRGVSLNLTRKFAQQM
CTALLFLATPELSIIHCDLKPENILLCNPKRSAIKIVDFGSSCQL
GQRIYQYIQSRFYRSPEVLLGMPYDLAIDMWSLGCILVEMHTGEP
LFSGANEVDQMNKIVEVLGIPPAHILDQAPKARKFFEKLPDGTWN
LKKTKDGKREYKPPGTRKLHNILGVETGGPGGRRAGESGHTVADY
LKFKDLILRMLDYDPKTRIQPYYALQHSFFKKTADEGTNTSNSVS
TSPAMEQSQSSGTTSSTSSSSGGAALDARCL
DYRKIA cDNA isoform 3
(SEQ ID NO: 26)
atgcatacaggaggagagacttcagcatgcaaaccttcatctgtt
cggcttgcaccgtcattttcattccatgctgctggccttcagat
ggctggacagatgccccattcacatcagtacagtgaccgtcgcca
gccaaacataagtgaccaacaggtttctgccttatcatattctga
ccagattcagcaacctctaactaaccaggtgatgcctgatattgt
catgttacagaggcggatgccccaaaccttccgtgacccagcaac
tgctcccctgagaaaactttctgttgacttgatcaaaacatacaa
gcatattaatgaggtttactatgcaaaaaagaagcgaagacacca
acagggccagggagacgattctagtcataagaaggaacggaaggt
ttacaatgatggttatgatgatgataactatgattatattgtaaa
aaacggagaaaagtggatggatcgttacgaaattgactccttgat
aggcaaaggttcctttggacaggttgtaaaggcatatgatcgtgt
ggagcaagaatgggttgccattaaaataataaagaacaagaaggc
ttttctgaatcaagcacagatagaagtgcgacttcttgagctcat
gaacaaacatgacactgaaatgaaatactacatagtgcatttgaa
acgccactttatgtttcgaaaccatctctgtttagtttttgaaat
gctgtcctacaacctctatgacttgctgagaaacaccaatttccg
aggggtctctttgaacctaacacgaaagtttgcgcaacagatgtg
cactgcactgcttttccttgcgactccagaacttagtatcattca
ctgtgatctaaaacctgaaaatatccttctttgtaaccccaaacg
cagtgcaatcaagatagttgactttggcagttcttgtcagttggg
gcagaggatataccagtatattcagagtcgcttttatcggtctcc
agaggtgctactgggaatgccttatgaccttgccattgatatgtg
gtccctcgggtgtattttggttgaaatgcacactggagaacctct
gttcagtggtgccaatgaggtagatcagatgaataaaatagtgga
agttctgggtattccacctgctcatattcttgaccaagcaccaaa
agcaagaaagttctttgagaagttgccagatggcacttggaactt
aaagaagaccaaagatggaaaacgggagtacaaaccaccaggaac
ccgtaaacttcataacattcttggagtggaaacaggaggacctgg
tgggcgacgtgctggggagtcaggtcatacggtcgctgactactt
gaagttcaaagacctcattttaaggatgcttgattatgaccccaa
aactcgaattcaaccttattatgctctgcagcacagtttcttcaa
gaaaacagctgatgaaggtacaaatacaagtaatagtgtatctac
aagccccgccatggagcagtctcagtcttcgggcaccacctccag
tacatcgtcaagctcaggtggagcagcactggatgccaggtgcct
ttag
DYRKIA protein isoform 4
(SEQ ID NO: 27)
MHTGGETSACKPSSVRLAPSFSFHAAGLQMAGQMPHSHQYSDRRQ
PNISDQQVSALSYSDQIQQPLTNQVMPDIVMLQRRMPQTFRDPA
TAPLRKLSVDLIKTYKHINEVYYAKKKRRHQQGQGDDSSHKKERK
VYNDGYDDDNYDYIVKNGEKWMDRYEIDSLIGKGSFGQVVKAYDR
VEQEWVAIKIIKNKKAFLNQAQIEVRLLELMNKHDTEMKYYIVHL
KRHFMFRNHLCLVFEMLSYNLYDLLRNTNFRGVSLNLTRKFAQQM
CTALLFLATPELSIIHCDLKPENILLCNPKRSAIKIVDFGSSCQL
GQRIYQYIQSRFYRSPEVLLGMPYDLAIDMWSLGCILVEMHTGEP
LFSGANEVDQMNKIVEVLGIPPAHILDQAPKARKFFEKLPDGTWN
LKKTKDGKREYKPPGTRKLHNILGVETGGPGGRRAGESGHTVADY
LKFKDLILRMLDYDPKTRIQPYYALQHSFFKKTADEGTNTSNSVS
TSPAMEQSQSSGTTSSTSSSSGGSSGTSNSGRARSDPTHQHRHSG
GHFTAAVQAMDCETHSPQVSSHVVHLLVSPAILRWSSTGCQVPLE
DYRKIA cDNA isoform 4
(SEQ ID NO: 28)
atgcatacaggaggagagacttcagcatgcaaaccttcatctgtt
cggcttgcaccgtcattttcattccatgctgctggccttcagat
ggctggacagatgccccattcacatcagtacagtgaccgtcgcca
gccaaacataagtgaccaacaggtttctgccttatcatattctga
ccagattcagcaacctctaactaaccaggtgatgcctgatattgt
catgttacagaggcggatgccccaaaccttccgtgacccagcaac
tgctcccctgagaaaactttctgttgacttgatcaaaacatacaa
gcatattaatgaggtttactatgcaaaaaagaagcgaagacacca
acagggccagggagacgattctagtcataagaaggaacggaaggt
ttacaatgatggttatgatgatgataactatgattatattgtaaa
aaacggagaaaagtggatggatcgttacgaaattgactccttgat
aggcaaaggttcctttggacaggttgtaaaggcatatgatcgtgt
ggagcaagaatgggttgccattaaaataataaagaacaagaaggc
ttttctgaatcaagcacagatagaagtgcgacttcttgagctcat
gaacaaacatgacactgaaatgaaatactacatagtgcatttgaa
acgccactttatgtttcgaaaccatctctgtttagtttttgaaat
gctgtcctacaacctctatgacttgctgagaaacaccaatttccg
aggggtctctttgaacctaacacgaaagtttgcgcaacagatgtg
cactgcactgcttttccttgcgactccagaacttagtatcattca
ctgtgatctaaaacctgaaaatatccttctttgtaaccccaaacg
cagtgcaatcaagatagttgactttggcagttcttgtcagttggg
gcagaggatataccagtatattcagagtcgcttttatcggtctcc
agaggtgctactgggaatgccttatgaccttgccattgatatgtg
gtccctcgggtgtattttggttgaaatgcacactggagaacctct
gttcagtggtgccaatgaggtagatcagatgaataaaatagtgga
agttctgggtattccacctgctcatattcttgaccaagcaccaaa
agcaagaaagttctttgagaagttgccagatggcacttggaactt
aaagaagaccaaagatggaaaacgggagtacaaaccaccaggaac
ccgtaaacttcataacattcttggagtggaaacaggaggacctgg
tgggcgacgtgctggggagtcaggtcatacggtcgctgactactt
gaagttcaaagacctcattttaaggatgcttgattatgaccccaa
aactcgaattcaaccttattatgctctgcagcacagtttcttcaa
gaaaacagctgatgaaggtacaaatacaagtaatagtgtatctac
aagccccgccatggagcagtctcagtcttcgggcaccacctccag
tacatcgtcaagctcaggtggctcatcggggacaagcaacagtgg
gagagcccggtcggatccgacgcaccagcatcggcacagtggtgg
gcacttcacagctgccgtgcaggccatggactgcgagacacacag
tccccaggtgagctcgcacgtggttcatttgcttgtgtcacctgc
cattctcaggtggagcagcactggatgccaggtgcctttagaatg
a
DYRK1B protein isoform 1
(SEQ ID NO: 29)
MAVPPGHGPFSGFPGPQEHTQVLPDVRLLPRRLPLAFRDATSAPL
RKLSVDLIKTYKHINEVYYAKKKRRAQQAPPQDSSNKKEKKVLN
HGYDDDNHDYIVRSGERWLERYEIDSLIGKGSFGQVVKAYDHQTQ
ELVAIKIIKNKKAFLNQAQIELRLLELMNQHDTEMKYYIVHLKRH
FMFRNHLCLVFELLSYNLYDLLRNTHFRGVSLNLTRKLAQQLCTA
LLFLATPELSIIHCDLKPENILLCNPKRSAIKIVDFGSSCQLGQR
IYQYIQSRFYRSPEVLLGTPYDLAIDMWSLGCILVEMHTGEPLFS
GSNEVDQMNRIVEVLGIPPAAMLDQAPKARKYFERLPGGGWTLRR
TKELRKDYQGPGTRRLQEVLGVQTGGPGGRRAGEPGHSPADYLRF
QDLVLRMLEYEPAARISPLGALQHGFFRRTADEATNTGPAGSSAS
TSPAPLDTCPSSSTASSISSSGGSSGSSSDNRTYRYSNRYCGGPG
PPITDCEMNSPQVPPSQPLRPWAGGDVPHKTHQAPASASSLPGTG
AQLPPQPRYLGRPPSPTSPPPPELMDVSLVGGPADCSPPHPAPAP
QHPAASALRTRMTGGRPPLPPPDDPATLGPHLGLRGVPQSTAASS
DYRK1B cDNA isoform 1
(SEQ ID NO: 30)
atggccgtcccaccgggccatggtcccttctctggcttcccaggg
ccccaggagcacacgcaggtattgcctgatgtgcggctactgcc
tcggaggctgcccctggccttccgggatgcaacctcagccccgct
gcgtaagctctctgtggacctcatcaagacctacaagcacatcaa
tgaggtatactatgcgaagaagaagcggcgggcccagcaggcgcc
accccaggattcgagcaacaagaaggagaagaaggtcctgaacca
tggttatgatgacgacaaccatgactacatcgtgcgcagtggcga
gcgctggctggagcgctacgaaattgactcgctcattggcaaagg
ctcctttggccaggtggtgaaagcctatgatcatcagacccagga
gcttgtggccatcaagatcatcaagaacaaaaaggctttcctgaa
ccaggcccagattgagctgcggctgctggagctgatgaaccagca
tgacacggagatgaagtactatatagtacacctgaagcggcactt
catgttccggaaccacctgtgcctggtatttgagctgctgtccta
caacctgtacgacctcctgcgcaacacccacttccgcggcgtctc
gctgaacctgacccggaagctggcgcagcagctctgcacggcact
gctctttctggccacgcctgagctcagcatcattcactgcgacct
caagcccgaaaacatcttgctgtgcaaccccaagcgcagcgccat
caagattgtggacttcggcagctcctgccagcttggccagaggat
ctaccagtatatccagagccgcttctaccgctcacctgaggtgct
cctgggcacaccctacgacctggccattgacatgtggtccctggg
ctgcatccttgtggagatgcacaccggagagcccctcttcagtgg
ctccaatgaggtcgaccagatgaaccgcattgtggaggtgctggg
catcccaccggccgccatgctggaccaggcgcccaaggctcgcaa
gtactttgaacggctgcctgggggtggctggaccctacgaaggac
gaaagaactcaggaaggattaccagggccccgggacacggcggct
gcaggaggtgctgggcgtgcagacgggcgggcccgggggccggcg
ggcgggggagccgggccacagccccgccgactacctccgcttcca
ggacctggtgctgcgcatgctggagtatgagcccgccgcccgcat
cagccccctgggggctctgcagcacggcttcttccgccgcacggc
cgacgaggccaccaacacgggcccggcaggcagcagtgcctccac
ctcgcccgcgcccctcgacacctgcccctcttccagcaccgccag
ctccatctccagttctggaggctccagtggctcctccagtgacaa
ccggacctaccgctacagcaaccgatattgtgggggccctgggcc
ccctatcacagactgtgagatgaacagcccccaggtcccaccctc
ccagccgctgcggccctgggcagggggtgatgtgccccacaagac
acatcaagcccctgcctctgcctcgtcactgcctgggaccggggc
ccagttacccccccagccccgataccttggtcgtcccccatcacc
aacctcaccaccacccccggagctgatggatgtgagcctggtggg
cggccctgctgactgctccccacctcacccagcgcctgcccccca
gcacccggctgcctcagccctccggactcggatgactggaggtcg
tccacccctcccgcctcctgatgaccctgccactctggggcctca
cctgggcctccgtggtgtaccccagagcacagcagccagctcgtg
a
DYRK1B protein isoform 2
(SEQ ID NO: 31)
MAVPPGHGPFSGFPGPQEHTQVLPDVRLLPRRLPLAFRDATSAPL
RKLSVDLIKTYKHINEVYYAKKKRRAQQAPPQDSSNKKEKKVLN
HGYDDDNHDYIVRSGERWLERYEIDSLIGKGSFGQVVKAYDHQTQ
ELVAIKIIKNKKAFLNQAQIELRLLELMNQHDTEMKYYIVHLKRH
FMFRNHLCLVFELLSYNLYDLLRNTHFRGVSLNLTRKLAQQLCTA
LLFLATPELSIIHCDLKPENILLCNPKRSAIKIVDFGSSCQLGQR
IYQYIQSRFYRSPEVLLGTPYDLAIDMWSLGCILVEMHTGEPLFS
GSNEVDQMNRIVEVLGIPPAAMLDQAPKARKYFERLPGGGWTLRR
TKELRKDLVLRMLEYEPAARISPLGALQHGFFRRTADEATNTGPA
GSSASTSPAPLDTCPSSSTASSISSSGGSSGSSSDNRTYRYSNRY
CGGPGPPITDCEMNSPQVPPSQPLRPWAGGDVPHKTHQAPASASS
LPGTGAQLPPQPRYLGRPPSPTSPPPPELMDVSLVGGPADCSPPH
PAPAPQHPAASALRTRMTGGRPPLPPPDDPATLGPHLGLRGVPQS
TAASS
DYRK1B cDNA isoform 2
(SEQ ID NO: 32)
atggccgtcccaccgggccatggtcccttctctggcttcccaggg
ccccaggagcacacgcaggtattgcctgatgtgcggctactgcc
tcggaggctgcccctggccttccgggatgcaacctcagccccgct
gcgtaagctctctgtggacctcatcaagacctacaagcacatcaa
tgaggtatactatgcgaagaagaagcggcgggcccagcaggcgcc
accccaggattcgagcaacaagaaggagaagaaggtcctgaacca
tggttatgatgacgacaaccatgactacatcgtgcgcagtggcga
gcgctggctggagcgctacgaaattgactcgctcattggcaaagg
ctcctttggccaggtggtgaaagcctatgatcatcagacccagga
gcttgtggccatcaagatcatcaagaacaaaaaggctttcctgaa
ccaggcccagattgagctgcggctgctggagctgatgaaccagca
tgacacggagatgaagtactatatagtacacctgaagcggcactt
catgttccggaaccacctgtgcctggtatttgagctgctgtccta
caacctgtacgacctcctgcgcaacacccacttccgcggcgtctc
gctgaacctgacccggaagctggcgcagcagctctgcacggcact
gctctttctggccacgcctgagctcagcatcattcactgcgacct
caagcccgaaaacatcttgctgtgcaaccccaagcgcagcgccat
caagattgtggacttcggcagctcctgccagcttggccagaggat
ctaccagtatatccagagccgcttctaccgctcacctgaggtgct
cctgggcacaccctacgacctggccattgacatgtggtccctggg
ctgcatccttgtggagatgcacaccggagagcccctcttcagtgg
ctccaatgaggtcgaccagatgaaccgcattgtggaggtgctggg
catcccaccggccgccatgctggaccaggcgcccaaggctcgcaa
gtactttgaacggctgcctgggggtggctggaccctacgaaggac
gaaagaactcaggaaggacctggtgctgcgcatgctggagtatga
gcccgccgcccgcatcagccccctgggggctctgcagcacggctt
cttccgccgcacggccgacgaggccaccaacacgggcccggcagg
cagcagtgcctccacctcgcccgcgcccctcgacacctgcccctc
ttccagcaccgccagctccatctccagttctggaggctccagtgg
ctcctccagtgacaaccggacctaccgctacagcaaccgatattg
tgggggccctgggccccctatcacagactgtgagatgaacagccc
ccaggtcccaccctcccagccgctgcggccctgggcagggggtga
tgtgccccacaagacacatcaagcccctgcctctgcctcgtcact
gcctgggaccggggcccagttacccccccagccccgataccttgg
tcgtcccccatcaccaacctcaccaccacccccggagctgatgga
tgtgagcctggtgggcggccctgctgactgctccccacctcaccc
agcgcctgccccccagcacccggctgcctcagccctccggactcg
gatgactggaggtcgtccacccctcccgcctcctgatgaccctgc
cactctggggcctcacctgggcctccgtggtgtaccccagagcac
agcagccagctcgtga
DYRK1B protein isoform 3
(SEQ ID NO: 33)
MAVPPGHGPFSGFPGPQEHTQVLPDVRLLPRRLPLAFRDATSAPL
RKLSVDLIKTYKHINEVYYAKKKRRAQQAPPQDSSNKKEKKVLN
HGYDDDNHDYIVRSGERWLERYEIDSLIGKGSFGQVVKAYDHQTQ
ELVAIKIIKNKKAFLNQAQIELRLLELMNQHDTEMKYYIVHLKRH
FMFRNHLCLVFELLSYNLYDLLRNTHFRGVSLNLTRKLAQQLCTA
LLFLATPELSIIHCDLKPENILLCNPKRSAIKIVDFGSSCQLGQR
IYQYIQSRFYRSPEVLLGTPYDLAIDMWSLGCILVEMHTGEPLFS
GSNEVDQMNRIVEVLGIPPAAMLDQAPKARKYFERLPGGGWTLRR
TKELRKDYQGPGTRRLQEDLVLRMLEYEPAARISPLGALQHGFFR
RTADEATNTGPAGSSASTSPAPLDTCPSSSTASSISSSGGSSGSS
SDNRTYRYSNRYCGGPGPPITDCEMNSPQVPPSQPLRPWAGGDVP
HKTHQAPASASSLPGTGAQLPPQPRYLGRPPSPTSPPPPELMDVS
LVGGPADCSPPHPAPAPQHPAASALRTRMTGGRPPLPPPDDPATL
GPHLGLRGVPQSTAASS
DYRK1B cDNA isoform 3
(SEQ ID NO: 34)
atggccgtcccaccgggccatggtcccttctctggcttcccaggg
ccccaggagcacacgcaggtattgcctgatgtgcggctactgcc
tcggaggctgcccctggccttccgggatgcaacctcagccccgct
gcgtaagctctctgtggacctcatcaagacctacaagcacatcaa
tgaggtatactatgcgaagaagaagcggcgggcccagcaggcgcc
accccaggattcgagcaacaagaaggagaagaaggtcctgaacca
tggttatgatgacgacaaccatgactacatcgtgcgcagtggcga
gcgctggctggagcgctacgaaattgactcgctcattggcaaagg
ctcctttggccaggtggtgaaagcctatgatcatcagacccagga
gcttgtggccatcaagatcatcaagaacaaaaaggctttcctgaa
ccaggcccagattgagctgcggctgctggagctgatgaaccagca
tgacacggagatgaagtactatatagtacacctgaagcggcactt
catgttccggaaccacctgtgcctggtatttgagctgctgtccta
caacctgtacgacctcctgcgcaacacccacttccgcggcgtctc
gctgaacctgacccggaagctggcgcagcagctctgcacggcact
gctctttctggccacgcctgagctcagcatcattcactgcgacct
caagcccgaaaacatcttgctgtgcaaccccaagcgcagcgccat
caagattgtggacttcggcagctcctgccagcttggccagaggat
ctaccagtatatccagagccgcttctaccgctcacctgaggtgct
cctgggcacaccctacgacctggccattgacatgtggtccctggg
ctgcatccttgtggagatgcacaccggagagcccctcttcagtgg
ctccaatgaggtcgaccagatgaaccgcattgtggaggtgctggg
catcccaccggccgccatgctggaccaggcgcccaaggctcgcaa
gtactttgaacggctgcctgggggtggctggaccctacgaaggac
gaaagaactcaggaaggattaccagggccccgggacacggcggct
gcaggaggacctggtgctgcgcatgctggagtatgagcccgccgc
ccgcatcagccccctgggggctctgcagcacggcttcttccgccg
cacggccgacgaggccaccaacacgggcccggcaggcagcagtgc
ctccacctcgcccgcgcccctcgacacctgcccctcttccagcac
cgccagctccatctccagttctggaggctccagtggctcctccag
tgacaaccggacctaccgctacagcaaccgatattgtgggggccc
tgggccccctatcacagactgtgagatgaacagcccccaggtccc
accctcccagccgctgcggccctgggcagggggtgatgtgcccca
caagacacatcaagcccctgcctctgcctcgtcactgcctgggac
cggggcccagttacccccccagccccgataccttggtcgtccccc
atcaccaacctcaccaccacccccggagctgatggatgtgagcct
ggtgggcggccctgctgactgctccccacctcacccagcgcctgc
cccccagcacccggctgcctcagccctccggactcggatgactgg
aggtcgtccacccctcccgcctcctgatgaccctgccactctggg
gcctcacctgggcctccgtggtgtaccccagagcacagcagccag
ctcgtga
DYRK2 protein isoform 1
(SEQ ID NO: 35)
MLTRKPSAAAPAAYPTGRGGDSAVRQLQASPGLGAGATRSGVGTG
PPSPIALPPLRASNAAAAAHTIGGSKHTMNDHLHVGSHAHGQIQ
VQQLFEDNSNKRTVLTTQPNGLTTVGKTGLPVVPERQLDSIHRRQ
GSSTSLKSMEGMGKVKATPMTPEQAMKQYMQKLTAFEHHEIFSYP
EIYFLGLNAKKRQGMTGGPNNGGYDDDQGSYVQVPHDHVAYRYEV
LKVIGKGSFGQVVKAYDHKVHQHVALKMVRNEKRFHRQAAEEIRI
LEHLRKQDKDNTMNVIHMLENFTFRNHICMTFELLSMNLYELIKK
NKFQGFSLPLVRKFAHSILQCLDALHKNRIIHCDLKPENILLKQQ
GRSGIKVIDFGSSCYEHQRVYTYIQSRFYRAPEVILGARYGMPID
MWSLGCILAELLTGYPLLPGEDEGDQLACMIELLGMPSQKLLDAS
KRAKNFVSSKGYPryctvttlsdgsvvlnggrsrrgklrgppesr
ewgnalkgcddplfldflkqclewdpAVRMTPGQALRHPWLRRRL
PKPPTGEKTSVKRITESTGAITSISKLPPPSSSASKLRTNLAQMT
DANGNIQQRTVLPKLVS
DYRK2 cDNA isoform 1
(SEQ ID NO: 36)
atgttaaccaggaaaccttcggccgccgctcccgccgcctacccg
accggccgaggtggggacagcgccgttcgtcagcttcaggcttc
cccggggctcggtgcaggggccacccggagcggagtggggactgg
cccgccctcccccatcgccctgccgcctctccgggccagcaacgc
tgccgccgcagcccacacgattggcggcagtaagcacacaatgaa
tgatcacctgcatgtcggcagccacgctcacggacagatccaggt
tcaacagttgtttgaggataacagtaacaagcggacagtgctcac
gacacaaccaaatgggcttacaacagtgggcaaaacgggcttgcc
agtggtgccagagcggcagctggacagcattcatagacggcaggg
gagctccacctctctaaagtccatggaaggcatggggaaggtgaa
agccacccccatgacacctgaacaagcaatgaagcaatacatgca
aaaactcacagccttcgaacaccatgagattttcagctaccctga
aatatatttcttgggtctaaatgctaagaagcgccagggcatgac
aggtgggcccaacaatggtggctatgatgatgaccagggatcata
tgtgcaggtgccccacgatcacgtggcttacaggtatgaggtcct
caaggtcattgggaaggggagctttgggcaggtggtcaaggccta
cgatcacaaagtccaccagcacgtggccctaaagatggtgcggaa
tgagaagcgcttccaccggcaagcagcggaggagatccgaatcct
ggaacacctgcggaagcaggacaaggataacacaatgaatgtcat
ccatatgctggagaatttcaccttccgcaaccacatctgcatgac
gtttgagctgctgagcatgaacctctatgagctcatcaagaagaa
taaattccagggcttcagtctgcctttggttcgcaagtttgccca
ctcgattctgcagtgcttggatgctttgcacaaaaacagaataat
tcactgtgaccttaagcccgagaacattttgttaaagcagcaggg
tagaagcggtattaaagtaattgattttggctccagttgttacga
gcatcagcgtgtctacacgtacatccagtcgcgtttttaccgggc
tccagaagtgatccttggggccaggtatggcatgcccattgatat
gtggagcctgggctgcattttagcagagctcctgacgggttaccc
cctcttgcctggggaagatgaaggggaccagctggcctgtatgat
tgaactgttgggcatgccctcacagaaactgctggatgcatccaa
acgagccaaaaattttgtgagctccaagggttatccccgttactg
cactgtcacgactctctcagatggctctgtggtcctaaacggagg
ccgttcccggagggggaaactgaggggcccaccggagagcagaga
gtgggggaacgcgctgaaggggtgtgatgatccccttttccttga
cttcttaaaacagtgtttagagtgggatcctgcagtgcgcatgac
cccaggccaggctttgcggcacccctggctgaggaggcggttgcc
aaagcctcccaccggggagaaaacgtcagtgaaaaggataactga
gagcaccggtgctatcacatctatatccaagttacctccaccttc
tagctcagcttccaaactgaggactaatttggcgcagatgacaga
tgccaatgggaatattcagcagaggacagtgttgccaaaacttgt
tagctga
DYRK2 protein isoform 2
(SEQ ID NO: 37)
MNDHLHVGSHAHGQIQVQQLFEDNSNKRTVLTTQPNGLTTVGKTG
LPVVPERQLDSIHRRQGSSTSLKSMEGMGKVKATPMTPEQAMKQ
YMQKLTAFEHHEIFSYPEIYFLGLNAKKRQGMTGGPNNGGYDDDQ
GSYVQVPHDHVAYRYEVLKVIGKGSFGQVVKAYDHKVHQHVALKM
VRNEKRFHRQAAEEIRILEHLRKQDKDNTMNVIHMLENFTFRNHI
CMTFELLSMNLYELIKKNKFQGFSLPLVRKFAHSILQCLDALHKN
RIIHCDLKPENILLKQQGRSGIKVIDFGSSCYEHQRVYTYIQSRF
YRAPEVILGARYGMPIDMWSLGCILAELLTGYPLLPGEDEGDQLA
CMIELLGMPSQKLLDASKRAKNFVSSKGYPRYCTVTTLSDGSVVL
NGGRSRRGKLRGPPESREWGNALKGCDDPLFLDFLKQCLEWDPAV
RMTPGQALRHPWLRRRLPKPPTGEKTSVKRITESTGAITSISKLP
PPSSSASKLRTNLAQMTDANGNIQQRTVLPKLVS
DYRK2 cDNA isoform 2
(SEQ ID NO: 38)
atgaatgatcacctgcatgtcggcagccacgctcacggacagatc
caggttcaacagttgtttgaggataacagtaacaagcggacagt
gctcacgacacaaccaaatgggcttacaacagtgggcaaaacggg
cttgccagtggtgccagagcggcagctggacagcattcatagacg
gcaggggagctccacctctctaaagtccatggaaggcatggggaa
ggtgaaagccacccccatgacacctgaacaagcaatgaagcaata
catgcaaaaactcacagccttcgaacaccatgagattttcagcta
ccctgaaatatatttcttgggtctaaatgctaagaagcgccaggg
catgacaggtgggcccaacaatggtggctatgatgatgaccaggg
atcatatgtgcaggtgccccacgatcacgtggcttacaggtatga
ggtcctcaaggtcattgggaaggggagctttgggcaggtggtcaa
ggcctacgatcacaaagtccaccagcacgtggccctaaagatggt
gcggaatgagaagcgcttccaccggcaagcagcggaggagatccg
aatcctggaacacctgcggaagcaggacaaggataacacaatgaa
tgtcatccatatgctggagatttcaccttccgcaaccacatctgc
atgacgtttgagctgctgagcatgaacctctatgagctcatcaag
aagaataaattccagggcttcagtctgcctttggttcgcaagttt
gcccactcgattctgcagtgcttggatgctttgcacaaaaacaga
ataattcactgtgaccttaagcccgagaacattttgttaaagcag
cagggtagaagcggtattaaagtaattgattttggctccagttgt
tacgagcatcagcgtgtctacacgtacatccagtcgcgtttttac
cgggctccagaagtgatccttggggccaggtatggcatgcccatt
gatatgtggagcctgggctgcattttagcagagctcctgacgggt
taccccctcttgcctggggaagatgaaggggaccagctggcctgt
atgattgaactgttgggcatgccctcacagaaactgctggatgca
tccaaacgagccaaaaattttgtgagctccaagggttatccccgt
tactgcactgtcacgactctctcagatggctctgtggtcctaaac
ggaggccgttcccggagggggaaactgaggggcccaccggagagc
agagagtgggggaacgcgctgaaggggtgtgatgatccccttttc
cttgacttcttaaaacagtgtttagagtgggatcctgcagtgcgc
atgaccccaggccaggctttgcggcacccctggctgaggaggcgg
ttgccaaagcctcccaccggggagaaaacgtcagtgaaaaggata
actgagagcaccggtgctatcacatctatatccaagttacctcca
ccttctagctcagcttccaaactgaggactaatttggcgcagatg
acagatgccaatgggaatattcagcagaggacagtgttgccaaaa
cttgttagctga
DYRK3 protein isoform 1
(SEQ ID NO: 39)
MGGTARGPGRKDAGPPGAGLPPQQRRLGDGVYDTFMMIDETKCPP
CSNVLCNPSEPPPPRRLNMTTEQFTGDHTQHFLDGGEMKVEQLF
QEFGNRKSNTIQSDGISDSEKCSPTVSQGKSSDCLNTVKSNSSSK
APKVVPLTPEQALKQYKHHLTAYEKLEIINYPEIYFVGPNAKKRH
GVIGGPNNGGYDDADGAYIHVPRDHLAYRYEVLKIIGKGSFGQVA
RVYDHKLRQYVALKMVRNEKRFHRQAAEEIRILEHLKKQDKTGSM
NVIHMLESFTFRNHVCMAFELLSIDLYELIKKNKFQGFSVQLVRK
FAQSILQSLDALHKNKIIHCDLKPENILLKHHGRSSTKVIDFGSS
CFEYQKLYTYIQSRFYRAPEIILGSRYSTPIDIWSFGCILAELLT
GQPLFPGEDEGDQLACMMELLGMPPPKLLEQSKRAKYFINSKGIP
RYCSVTTQADGRVVLVGGRSRRGKKRGPPGSKDWGTALKGCDDYL
FIEFLKRCLHWDPSARLTPAQALRHPWISKSVPRPLTTIDKVSGK
RVVNPASAFQGLGSKLPPVVGIANKLKANLMSETNGSIPLCSVLP
KLIS
DYRK3 cDNA isoform 1
(SEQ ID NO: 40)
atgggaggcacagctcgtgggcctgggcggaaggatgcggggccg
cctggggccgggctcccgccccagcagcggaggttgggggatgg
tgtctatgacaccttcatgatgatagatgaaaccaaatgtccccc
ctgttcaaatgtactctgcaatccttctgaaccacctccacccag
aagactaaatatgaccactgagcagtttacaggagatcatactca
gcactttttggatggaggtgagatgaaggtagaacagctgtttca
agaatttggcaacagaaaatccaatactattcagtcagatggcat
cagtgactctgaaaaatgctctcctactgtttctcagggtaaaag
ttcagattgcttgaatacagtaaaatccaacagttcatccaaggc
acccaaagtggtgcctctgactccagaacaagccctgaagcaata
taaacaccacctcactgcctatgagaaactggaaataattaatta
tccagaaatttactttgtaggtccaaatgccaagaaaagacatgg
agttattggtggtcccaataatggagggtatgatgatgcagatgg
ggcctatattcatgtacctcgagaccatctagcttatcgatatga
ggtgctgaaaattattggcaaggggagttttgggcaggtggccag
ggtctatgatcacaaacttcgacagtacgtggccctaaaaatggt
gcgcaatgagaagcgctttcatcgtcaagcagctgaggagatccg
gattttggagcatcttaagaaacaggataaaactggtagtatgaa
cgttatccacatgctggaaagtttcacattccggaaccatgtttg
catggcctttgaattgctgagcatagacctttatgagctgattaa
aaaaaataagtttcagggttttagcgtccagttggtacgcaagtt
tgcccagtccatcttgcaatctttggatgccctccacaaaaataa
gattattcactgcgatctgaagccagaaaacattctcctgaaaca
ccacgggcgcagttcaaccaaggtcattgactttgggtccagctg
tttcgagtaccagaagctctacacatatatccagtctcggttcta
cagagctccagaaatcatcttaggaagccgctacagcacaccaat
tgacatatggagttttggctgcatccttgcagaacttttaacagg
acagcctctcttccctggagaggatgaaggagaccagttggcctg
catgatggagcttctagggatgccaccaccaaaacttctggagca
atccaaacgtgccaagtactttattaattccaagggcataccccg
ctactgctctgtgactacccaggcagatgggagggttgtgcttgt
ggggggtcgctcacgtaggggtaaaaagcggggtcccccaggcag
caaagactgggggacagcactgaaagggtgtgatgactacttgtt
tatagagttcttgaaaaggtgtcttcactgggacccctctgcccg
cttgaccccagctcaagcattaagacacccttggattagcaagtc
tgtccccagacctctcaccaccatagacaaggtgtcagggaaacg
ggtagttaatcctgcaagtgctttccagggattgggttctaagct
gcctccagttgttggaatagccaataagcttaaagctaacttaat
gtcagaaaccaatggtagtatacccctatgcagtgtattgccaaa
actgattagctag
DYRK3 protein isoform 2
(SEQ ID NO: 41)
MKWKEKLGDGVYDTFMMIDETKCPPCSNVLCNPSEPPPPRRLNMT
TEQFTGDHTQHFLDGGEMKVEQLFQEFGNRKSNTIQSDGISDSE
KCSPTVSQGKSSDCLNTVKSNSSSKAPKVVPLTPEQALKQYKHHL
TAYEKLEIINYPEIYFVGPNAKKRHGVIGGPNNGGYDDADGAYIH
VPRDHLAYRYEVLKIIGKGSFGQVARVYDHKLRQYVALKMVRNEK
RFHRQAAEEIRILEHLKKQDKTGSMNVIHMLESFTFRNHVCMAFE
LLSIDLYELIKKNKFQGFSVQLVRKFAQSILQSLDALHKNKIIHC
DLKPENILLKHHGRSSTKVIDFGSSCFEYQKLYTYIQSRFYRAPE
IILGSRYSTPIDIWSFGCILAELLTGQPLFPGEDEGDQLACMMEL
LGMPPPKLLEQSKRAKYFINSKGIPRYCSVTTQADGRVVLVGGRS
RRGKKRGPPGSKDWGTALKGCDDYLFIEFLKRCLHWDPSARLTPA
QALRHPWISKSVPRPLTTIDKVSGKRVVNPASAFQGLGSKLPPVV
GIANKLKANLMSETNGSIPLCSVLPKLIS
DYRK3 cDNA isoform 2
(SEQ ID NO: 42)
atgaagtggaaagagaagttgggggatggtgtctatgacaccttc
atgatgatagatgaaaccaaatgtcccccctgttcaaatgtact
ctgcaatccttctgaaccacctccacccagaagactaaatatgac
cactgagcagtttacaggagatcatactcagcactttttggatgg
aggtgagatgaaggtagaacagctgtttcaagaatttggcaacag
aaaatccaatactattcagtcagatggcatcagtgactctgaaaa
atgctctcctactgtttctcagggtaaaagttcagattgcttgaa
tacagtaaaatccaacagttcatccaaggcacccaaagtggtgcc
tctgactccagaacaagccctgaagcaatataaacaccacctcac
tgcctatgagaaactggaaataattaattatccagaaatttactt
tgtaggtccaaatgccaagaaaagacatggagttattggtggtcc
caataatggagggtatgatgatgcagatggggcctatattcatgt
acctcgagaccatctagcttatcgatatgaggtgctgaaaattat
tggcaaggggagttttgggcaggtggccagggtctatgatcacaa
acttcgacagtacgtggccctaaaaatggtgcgcaatgagaagcg
ctttcatcgtcaagcagctgaggagatccggattttggagcatct
taagaaacaggataaaactggtagtatgaacgttatccacatgct
ggaaagtttcacattccggaaccatgtttgcatggcctttgaatt
gctgagcatagacctttatgagctgattaaaaaaaataagtttca
gggttttagcgtccagttggtacgcaagtttgcccagtccatctt
gcaatctttggatgccctccacaaaaataagattattcactgcga
tctgaagccagaaaacattctcctgaaacaccacgggcgcagttc
aaccaaggtcattgactttgggtccagctgtttcgagtaccagaa
gctctacacatatatccagtctcggttctacagagctccagaaat
catcttaggaagccgctacagcacaccaattgacatatggagttt
tggctgcatccttgcagaacttttaacaggacagcctctcttccc
tggagaggatgaaggagaccagttggcctgcatgatggagcttct
agggatgccaccaccaaaacttctggagcaatccaaacgtgccaa
gtactttattaattccaagggcataccccgctactgctctgtgac
tacccaggcagatgggagggttgtgcttgtggggggtcgctcacg
taggggtaaaaagcggggtcccccaggcagcaaagactgggggac
agcactgaaagggtgtgatgactacttgtttatagagttcttgaa
aaggtgtcttcactgggacccctctgcccgcttgaccccagctca
agcattaagacacccttggattagcaagtctgtccccagacctct
caccaccatagacaaggtgtcagggaaacgggtagttaatcctgc
aagtgctttccagggattgggttctaagctgcctccagttgttgg
aatagccaataagcttaaagctaacttaatgtcagaaaccaatgg
tagtatacccctatgcagtgtattgccaaaactgattagctag
DYRK4 protein isoform 1
(SEQ ID NO: 43)
MPASELKASEIPFHPSIKTQDPKAEEKSPKKQKVTLTAAEALKLF
KNQLSPYEQSEILGYAELWFLGLEAKKLDTAPEKFSKTSFDDEH
GFYLKVLHDHIAYRYEVLETIGKGSFGQVAKCLDHKNNELVALKI
IRNKKRFHQQALMELKILEALRKKDKDNTYNVVHMKDFFYFRNHF
CITFELLGINLYELMKNNNFQGFSLSIVRRFTLSVLKCLQMLSVE
KIIHCDLKPENIVLYQKGQASVKVIDFGSSCYEHQKVYTYIQSRF
YRSPEVILGHPYDVAIDMWSLGCITAELYTGYPLFPGENEVEQLA
CIMEVLGLPPAGFIQTASRRQTFFDSKGFPKNITNNRGKKRYPDS
KDLTMVLKTYDTSFLDFLRRCLVWEPSLRMTPDQALKHAWIHQSR
NLKPQPRPQTLRKSNSFFPSETRKDKVQGCHHSSRKADEITKETT
EKTKDSPTKHVQHSGDQQDCLQHGADTVQLPQLVDAPKKSEAAVG
AEVSMTSPGQSKNFSLKNTNVLPPIV
DYRK4 cDNA isoform 1
(SEQ ID NO: 44)
atgccggcctcagagctcaaggcttcagaaatacctttccaccct
agcattaaaacccaggatcccaaggcagaggagaagtcaccaaa
gaagcaaaaggtgactctgacagcggcagaggccctaaagctttt
taagaaccagctgtctccatatgaacaaagtgaaatcctgggcta
cgcggagctgtggttcctgggtcttgaagccaagaagctcgacac
ggctcctgagaaatttagcaagacgagttttgatgatgagcatgg
cttctatctgaaggtcctgcatgatcacattgcctaccgctatga
agttctggagacaatcgggaaggggtcctttggacaggtggccaa
gtgcttggatcacaaaaacaatgagctggtggccctgaaaatcat
caggaacaagaagaggtttcaccagcaggccctgatggagctgaa
gatcctggaagctctcagaaagaaggacaaagacaacacctacaa
tgtggtgcatatgaaggactttttctactttcgcaatcacttctg
catcacctttgagctcctgggaatcaacttgtatgagttgatgaa
gaataacaactttcaaggcttcagtctgtccatagttcggcgctt
cactctctctgttttgaagtgcttgcagatgctttcggtagagaa
aatcattcactgtgatctcaagcccgaaaatatagtgctatacca
aaagggccaagcctctgttaaagtcattgactttggatcaagctg
ttatgaacaccagaaagtatacacgtacatccaaagccggttcta
ccgatccccagaagtgatcctgggccacccctacgacgtggccat
tgacatgtggagcctgggctgcatcacggcggagttgtacacggg
ctaccccctgttccccggggagaatgaggtggagcagctggcctg
catcatggaggtgctgggtctgccgccagccggcttcattcagac
agcctccaggagacagacattctttgattccaaaggttttcctaa
aaatataaccaacaacagggggaaaaaaagatacccagattccaa
ggacctcacgatggtgctgaaaacctatgacaccagcttcctgga
ctttctcagaaggtgtttggtatgggaaccttctcttcgcatgac
cccggaccaggccctcaagcatgcttggattcatcagtctcggaa
cctcaagccacagcccaggccccagaccctgaggaaatccaattc
ctttttcccctctgagacaaggaaggacaaggttcaaggctgtca
tcactcgagcagaaaagcagatgagatcaccaaagagactacaga
gaaaacaaaagatagccccacgaagcatgttcagcattcaggtga
tcagcaggactgtctccagcacggagctgacactgttcagctgcc
tcaactggtagacgctcccaagaagtcagaggcagctgtcggggc
ggaggtgtccatgacctccccaggacagagcaaaaacttctccct
caagaacacaaacgttttaccccctattgtatga
DYRK4 protein isoform 2
(SEQ ID NO: 45)
MPASELKASEIPFHPSIKTQDPKAEEKSPKKQKVTLTAAEALKLF
KNQLSPYEQSEILGYAELWFLGLEAKKLDTAPEKFSKTSFDDEH
GFYLKVLHDHIAYRYEVLETIGKGSFGQVAKCLDHKNNELVALKI
IRNKKRFHQQALMELKILEALRKKDKDNTYNVVHMKDFFYFRNHF
CITFELLGINLYELMKNNNFQGFSLSIVRRFTLSVLKCLQMLSVE
KIIHCDLKPENIVLYQKGQASVKVIDFGSSCYEHQKVYTYIQSRF
YRSPEVILGHPYDVAIDMWSLGCITAELYTGYPLFPGENEVEQLA
CIMEVLGLPPAGFIQTASRRQTFFDSKGFPKNITNNRGKKRYPDS
KDLTMVLKTYDTSFLDFLRRCLVWEPSLRMTPDQALKHAWIHQSR
NLKPQPRPQTLRKSNSFFPSETRKDKVQGCHHSSRKDEITKETTE
KTKDSPTKHVQHSGDQQDCLQHGADTVQLPQLVDAPKKSEAAVGA
EVSMTSPGQSKNFSLKNTNVLPPIV
DYRK4 cDNA isoform 2
(SEQ ID NO: 46)
atgccggcctcagagctcaaggcttcagaaatacctttccaccct
agcattaaaacccaggatcccaaggcagaggagaagtcaccaaa
gaagcaaaaggtgactctgacagcggcagaggccctaaagctttt
taagaaccagctgtctccatatgaacaaagtgaaatcctgggcta
cgcggagctgtggttcctgggtcttgaagccaagaagctcgacac
ggctcctgagaaatttagcaagacgagttttgatgatgagcatgg
cttctatctgaaggtcctgcatgatcacattgcctaccgctatga
agttctggagacaatcgggaaggggtcctttggacaggtggccaa
gtgcttggatcacaaaaacaatgagctggtggccctgaaaatcat
caggaacaagaagaggtttcaccagcaggccctgatggagctgaa
gatcctggaagctctcagaaagaaggacaaagacaacacctacaa
tgtggtgcatatgaaggactttttctactttcgcaatcacttctg
catcacctttgagctcctgggaatcaacttgtatgagttgatgaa
gaataacaactttcaaggcttcagtctgtccatagttcggcgctt
cactctctctgttttgaagtgcttgcagatgctttcggtagagaa
aatcattcactgtgatctcaagcccgaaaatatagtgctatacca
aaagggccaagcctctgttaaagtcattgactttggatcaagctg
ttatgaacaccagaaagtatacacgtacatccaaagccggttcta
ccgatccccagaagtgatcctgggccacccctacgacgtggccat
tgacatgtggagcctgggctgcatcacggcggagttgtacacggg
ctaccccctgttccccggggagaatgaggtggagcagctggcctg
catcatggaggtgctgggtctgccgccagccggcttcattcagac
agcctccaggagacagacattctttgattccaaaggttttcctaa
aaatataaccaacaacagggggaaaaaaagatacccagattccaa
ggacctcacgatggtgctgaaaacctatgacaccagcttcctgga
ctttctcagaaggtgtttggtatgggaaccttctcttcgcatgac
cccggaccaggccctcaagcatgcttggattcatcagtctcggaa
cctcaagccacagcccaggccccagaccctgaggaaatccaattc
ctttttcccctctgagacaaggaaggacaaggttcaaggctgtca
tcactcgagcagaaaagatgagatcaccaaagagactacagagaa
aacaaaagatagccccacgaagcatgttcagcattcaggtgatca
gcaggactgtctccagcacggagctgacactgttcagctgcctca
actggtagacgctcccaagaagtcagaggcagctgtcggggcgga
ggtgtccatgacctccccaggacagagcaaaaacttctccctcaa
gaacacaaacgttttaccccctattgtatga
DYRK4 protein isoform 3
(SEQ ID NO: 47)
MQLLPPPIRTGTKTQMDAKKPRKCDLTPFLVLKARKKQKFTSAKV
GSKLSVQIQKPPSNIKNSRMTQVFHKNTSVTSLPFVDTKGKKNT
VSFPHISKKVLLKSSLLYQENQAHNQMPASELKASEIPFHPSIKT
QDPKAEEKSPKKQKVTLTAAEALKLFKNQLSPYEQSEILGYAELW
FLGLEAKKLDTAPEKFSKTSFDDEHGFYLKVLHDHIAYRYEVLET
IGKGSFGQVAKCLDHKNNELVALKIIRNKKRFHQQALMELKILEA
LRKKDKDNTYNVVHMKDFFYFRNHFCITFELLGINLYELMKNNNF
QGFSLSIVRRFTLSVLKCLQMLSVEKIIHCDLKPENIVLYQKGQA
SVKVIDFGSSCYEHQKVYTYIQSRFYRSPEVILGHPYDVAIDMWS
LGCITAELYTGYPLFPGENEVEQLACIMEVLGLPPAGFIQTASRR
QTFFDSKGFPKNITNNRGKKRYPDSKDLTMVLKTYDTSFLDFLRR
CLVWEPSLRMTPDQALKHAWIHQSRNLKPQPRPQTLRKSNSFFPS
ETRKDKVQGCHHSSRKADEITKETTEKTKDSPTKHVQHSGDQQDC
LQHGADTVQLPQLVDAPKKSEAAVGAEVSMTSPGQSKNFSLKNTN
VLPPIV
DYRK4 protein isoform 4
(SEQ ID NO: 48)
MQLLPPPIRTGTKTQMDAKKPRKCDLTPFLVLKARKKQKFTSAKG
PTLSEIYMVGSKLSVQIQKPPSNIKNSRMTQVFHKNTSVTSLPF
VDTKGKKNTVSFPHISKKVLLKSSLLYQENQAHNQMPASELKASE
IPFHPSIKTQDPKAEEKSPKKQKVTLTAAEALKLFKNQLSPYEQS
EILGYAELWFLGLEAKKLDTAPEKFSKTSFDDEHGFYLKVLHDHI
AYRYEVLETIGKGSFGQVAKCLDHKNNELVALKIIRNKKRFHQQA
LMELKILEALRKKDKDNTYNVVHMKDFFYFRNHFCITFELLGINL
YELMKNNNFQGFSLSIVRRFTLSVLKCLQMLSVEKIIHCDLKPEN
IVLYQKGQASVKVIDFGSSCYEHQKVYTYIQSRFYRSPEVILGHP
YDVAIDMWSLGCITAELYTGYPLFPGENEVEQLACIMEVLGLPPA
GFIQTASRRQTFFDSKGFPKNITNNRGKKRYPDSKDLTMVLKTYD
TSFLDFLRRCLVWEPSLRMTPDQALKHAWIHQSRNLKPQPRPQTL
RKSNSFFPSETRKDKVQGCHHSSRKADEITKETTEKTKDSPTKHV
QHSGDQQDCLQHGADTVQLPQLVDAPKKSEAAVGAEVSMTSPGQS
KNFSLKNTNVLPPIV
DYRK4 protein isoform 5
(SEQ ID NO: 49)
MPASELKASEIPFHPSIKTQDPKAEEKSPKKQKVTLTAAEALKLF
KNQLSPYEQSEILGYAELWFLGLEAKKLDTAPEKFSKTSFDDEHG
FYLKVLHDHIAYRYEVLETIGKGSFGQVAKCLDHKNNELVALKII
RNKKRFHQQALMELKILEALRKKDKDNTYNVVHMKDFFYFRNHFC
ITFELLGINLYELMKNNNFQGFSLSIVRRFTLSVLKCLQMLSVEK
IIHCDLKPENIVLYQKGQASVKVIDFGSSCYEHQKVYTYIQSRFY
RSPEVILGHPYDVAIDMWSLGCITAELYTGYPLFPGENEVEQLAC
IMEVLGLPPAGFIQTASRRQTFFDSKGFPKNITNNRGKKRYPDSK
DLTMVLKTYDTSFLDFLRRWEPSLRMTPDQALKHAWIHQSRNLKP
QPRPQTLRKSNSFFPSETRKDKVQGCHHSSRKADEITKETTEKTK
DSPTKHVQHSGDQQDCLQHGADTVQLPQLVDAPKKSEAAVGAEVS
MTSPGQSKNFSLKNTNVLPPIV

### Exemplary DYRK Inhibitors

In some embodiments, the DYRK inhibitor inhibits one or more of the DYRK family members DYRK1A, DYRK1B, DYRK2, DYRK3, and DYRK4. In some embodiments, the DYRK inhibitor is a broad spectrum inhibitor, inhibiting two or more of the DYRK family members DYRK1A, DYRK1B, DYRK2, DYRK3, and DYRK4. In some embodiments, the DYRK inhibitor has an IC₅₀ of between about 100 pM and about 10 μM (e.g., between about 100 pM and about 9 μM, between about 100 pM and about 8 μM, between about 100 pM and about 7 μM, between about 100 pM and about 6 μM, between about 100 pM and about 5 μM, between about 100 pM and about 4 μM, between about 100 pM and about 3 μM, between about 100 pM and about 2 μM, between about 100 pM and about 1 μM, between about 100 pM and about 950 nM, between about 100 pM and about 900 nM, between about 100 pM and about 850 nM, between about 100 pM and about 800 nM, between about 100 pM and about 750 nM, between about 100 pM and about 700 nM, between about 100 pM and about 650 nM, between about 100 pM and about 600 nM, between about 100 pM and about 550 nM, between about 100 pM and about 500 nM, between about 100 pM and about 450 nM, between about 100 pM and about 400 nM, between about 100 pM and about 350 nM, between about 100 pM and about 300 nM, between about 100 pM and about 250 nM, between about 100 pM and about 200 nM, between about 100 pM and about 150 nM, between about 100 pM and about 100 nM, between about 100 pM and about 95 nM, between about 100 pM and about 90 nM, between about 100 pM and about 85 nM, between about 100 pM and about 80 nM, between about 100 pM and about 75 nM, between about 100 pM and about 70 nM, between about 100 pM and about 65 nM, between about 100 pM and about 60 nM, between about 100 pM and about 55 nM, between about 100 pM and about 50 nM, between about 100 pM and about 45 nM, between about 100 pM and about 40 nM, between about 100 pM and about 35 nM, between about 100 pM and about 30 nM, between about 100 pM and about 25 nM, between about 100 pM and about 20 nM, between about 100 pM and about 15 nM, between about 100 pM and about 10 nM, between about 100 pM and about 5 nM, between about 100 pM and about 4 nM, between about 100 pM and about 3 nM, between about 100 pM and about 2 nM, e.g., between about 1 nM and about 9 μM, between about 1 nM and about 8 μM, between about 1 nM and about 7 μM, between about 1 nM and about 6 μM, between about 1 nM and about 5 μM, between about 1 nM and about 4 μM, between about 1 nM and about 3 μM, between about 1 nM and about 2 μM, between about 1 nM and about 1 μM, between about 1 nM and about 950 nM, between about 1 nM and about 900 nM, between about 1 nM and about 850 nM, between about 1 nM and about 800 nM, between about 1 nM and about 750 nM, between about 1 nM and about 700 nM, between about 1 nM and about 650 nM, between about 1 nM and about 600 nM, between about 1 nM and about 550 nM, between about 1 nM and about 500 nM, between about 1 nM and about 450 nM, between about 1 nM and about 400 nM, between about 1 nM and about 350 nM, between about 1 nM and about 300 nM, between about 1 nM and about 250 nM, between about 1 nM and about 200 nM, between about 1 nM and about 150 nM, between about 1 nM and about 100 nM, between about 1 nM and about 95 nM, between about 1 nM and about 90 nM, between about 1 nM and about 85 nM, between about 1 nM and about 80 nM, between about 1 nM and about 75 nM, between about 1 nM and about 70 nM, between about 1 nM and about 65 nM, between about 1 nM and about 60 nM, between about 1 nM and about 55 nM, between about 1 nM and about 50 nM, between about 1 nM and about 45 nM, between about 1 nM and about 40 nM, between about 1 nM and about 35 nM, between about 1 nM and about 30 nM, between about 1 nM and about 25 nM, between about 1 nM and about 20 nM, between about 1 nM and about 15 nM, between about 1 nM and about 10 nM, between about 1 nM and about 5 nM, between about 1 nM and about 4 nM, between about 1 nM and about 3 nM, between about 1 nM and about 2 nM, between about 2 nM and about 10 μM, between about 2 nM and about 9 μM, between about 2 nM and about 8 μM, between about 2 nM and about 7 μM, between about 2 nM and about 6 μM, between about 2 nM and about 5 μM, between about 2 nM and about 4 μM, between about 2 nM and about 3 μM, between about 2 nM and about 2 μM, between about 2 nM and about 1 μM, between about 2 nM and about 950 nM, between about 2 nM and about 900 nM, between about 2 nM and about 850 nM, between about 2 nM and about 800 nM, between about 2 nM and about 750 nM, between about 2 nM and about 700 nM, between about 2 nM and about 650 nM, between about 2 nM and about 600 nM, between about 2 nM and about 550 nM, between about 2 nM and about 500 nM, between about 2 nM and about 450 nM, between about 2 nM and about 400 nM, between about 2 nM and about 350 nM, between about 2 nM and about 300 nM, between about 2 nM and about 250 nM, between about 2 nM and about 200 nM, between about 2 nM and about 150 nM, between about 2 nM and about 100 nM, between about 2 nM and about 95 nM, between about 2 nM and about 90 nM, between about 2 nM and about 85 nM, between about 2 nM and about 80 nM, between about 2 nM and about 75 nM, between about 2 nM and about 70 nM, between about 2 nM and about 65 nM, between about 2 nM and about 60 nM, between about 2 nM and about 55 nM, between about 2 nM and about 50 nM, between about 2 nM and about 45 nM, between about 2 nM and about 40 nM, between about 2 nM and about 35 nM, between about 2 nM and about 30 nM, between about 2 nM and about 25 nM, between about 2 nM and about 20 nM, between about 2 nM and about 15 nM, between about 2 nM and about 10 nM, between about 2 nM and about 5 nM, between about 2 nM and about 4 nM, between about 2 nM and about 3 nM, between about 5 nM and about 10 μM, between about 5 nM and about 9 μM, between about 5 nM and about 8 μM, between about 5 nM and about 7 μM, between about 5 nM and about 6 μM, between about 5 nM and about 5 μM, between about 5 nM and about 4 μM, between about 5 nM and about 3 μM, between about 5 nM and about 2 μM, between about 5 nM and about 1 μM, between about 5 nM and about 950 nM, between about 5 nM and about 900 nM, between about 5 nM and about 850 nM, between about 5 nM and about 800 nM, between about 5 nM and about 750 nM, between about 5 nM and about 700 nM, between about 5 nM and about 650 nM, between about 5 nM and about 600 nM, between about 5 nM and about 550 nM, between about 5 nM and about 500 nM, between about 5 nM and about 450 nM, between about 5 nM and about 400 nM, between about 5 nM and about 350 nM, between about 5 nM and about 300 nM, between about 5 nM and about 250 nM, between about 5 nM and about 200 nM, between about 5 nM and about 150 nM, between about 5 nM and about 100 nM, between about 5 nM and about 95 nM, between about 5 nM and about 90 nM, between about 5 nM and about 85 nM, between about 5 nM and about 80 nM, between about 5 nM and about 75 nM, between about 5 nM and about 70 nM, between about 5 nM and about 65 nM, between about 5 nM and about 60 nM, between about 5 nM and about 55 nM, between about 5 nM and about 50 nM, between about 5 nM and about 45 nM, between about 5 nM and about 40 nM, between about 5 nM and about 35 nM, between about 5 nM and about 30 nM, between about 5 nM and about 25 nM, between about 5 nM and about 20 nM, between about 5 nM and about 15 nM, between about 5 nM and about 10 nM, between about 10 nM and about 10 μM, between about 10 nM and about 9 μM, between about 10 nM and about 8 μM, between about 10 nM and about 7 μM, between about 10 nM and about 6 μM, between about 10 nM and about 5 μM, between about 10 nM and about 4 μM, between about 10 nM and about 3 μM, between about 10 nM and about 2 μM, between about 10 nM and about 1 μM, between about 10 nM and about 950 nM, between about 10 nM and about 900 nM, between about 10 nM and about 850 nM, between about 10 nM and about 800 nM, between about 10 nM and about 750 nM, between about 10 nM and about 700 nM, between about 10 nM and about 650 nM, between about 10 nM and about 600 nM, between about 10 nM and about 550 nM, between about 10 nM and about 500 nM, between about 10 nM and about 450 nM, between about 10 nM and about 400 nM, between about 10 nM and about 350 nM, between about 10 nM and about 300 nM, between about 10 nM and about 250 nM, between about 10 nM and about 200 nM, between about 10 nM and about 150 nM, between about 10 nM and about 100 nM, between about 10 nM and about 95 nM, between about 10 nM and about 90 nM, between about 10 nM and about 85 nM, between about 10 nM and about 80 nM, between about 10 nM and about 75 nM, between about 10 nM and about 70 nM, between about 10 nM and about 65 nM, between about 10 nM and about 60 nM, between about 10 nM and about 55 nM, between about 10 nM and about 50 nM, between about 10 nM and about 45 nM, between about 10 nM and about 40 nM, between about 10 nM and about 35 nM, between about 10 nM and about 30 nM, between about 10 nM and about 25 nM, between about 10 nM and about 20 nM, between about 10 nM and about 15 nM, between about 50 nM and about 10 μM, between about 50 nM and about 9 μM, between about 50 nM and about 8 μM, between about 50 nM and about 7 μM, between about 50 nM and about 6 μM, between about 50 nM and about 5 μM, between about 50 nM and about 4 μM, between about 50 nM and about 3 μM, between about 50 nM and about 2 μM, between about 50 nM and about 1 μM, between about 50 nM and about 950 nM, between about 50 nM and about 900 nM, between about 50 nM and about 850 nM, between about 50 nM and about 800 nM, between about 50 nM and about 750 nM, between about 50 nM and about 700 nM, between about 50 nM and about 650 nM, between about 50 nM and about 600 nM, between about 50 nM and about 550 nM, between about 50 nM and about 500 nM, between about 50 nM and about 450 nM, between about 50 nM and about 400 nM, between about 50 nM and about 350 nM, between about 50 nM and about 300 nM, between about 50 nM and about 250 nM, between about 50 nM and about 200 nM, between about 50 nM and about 150 nM, between about 50 nM and about 100 nM, between about 50 nM and about 95 nM, between about 50 nM and about 90 nM, between about 50 nM and about 85 nM, between about 50 nM and about 80 nM, between about 50 nM and about 75 nM, between about 50 nM and about 70 nM, between about 50 nM and about 65 nM, between about 50 nM and about 60 nM, between about 50 nM and about 55 nM, between about 100 nM and about 10 μM, between about 100 nM and about 9 μM, between about 100 nM and about 8 μM, between about 100 nM and about 7 μM, between about 100 nM and about 6 μM, between about 100 nM and about 5 μM, between about 100 nM and about 4 μM, between about 100 nM and about 3 μM, between about 100 nM and about 2 μM, between about 100 nM and about 1 μM, between about 100 nM and about 950 nM, between about 100 nM and about 900 nM, between about 100 nM and about 850 nM, between about 100 nM and about 800 nM, between about 100 nM and about 750 nM, between about 100 nM and about 700 nM, between about 100 nM and about 650 nM, between about 100 nM and about 600 nM, between about 100 nM and about 550 nM, between about 100 nM and about 500 nM, between about 100 nM and about 450 nM, between about 100 nM and about 400 nM, between about 100 nM and about 350 nM, between about 100 nM and about 300 nM, between about 100 nM and about 250 nM, between about 100 nM and about 200 nM, between about 100 nM and about 150 nM, between about 200 nM and about 10 μM, between about 200 nM and about 9 μM, between about 200 nM and about 8 μM, between about 200 nM and about 7 μM, between about 200 nM and about 6 μM, between about 200 nM and about 5 μM, between about 200 nM and about 4 μM, between about 200 nM and about 3 μM, between about 200 nM and about 2 μM, between about 200 nM and about 1 μM, between about 200 nM and about 950 nM, between about 200 nM and about 900 nM, between about 200 nM and about 850 nM, between about 200 nM and about 800 nM, between about 200 nM and about 750 nM, between about 200 nM and about 700 nM, between about 200 nM and about 650 nM, between about 200 nM and about 600 nM, between about 200 nM and about 550 nM, between about 200 nM and about 500 nM, between about 200 nM and about 450 nM, between about 200 nM and about 400 nM, between about 200 nM and about 350 nM, between about 200 nM and about 300 nM, between about 200 nM and about 250 nM, between about 250 nM and about 10 μM, between about 250 nM and about 9 μM, between about 250 nM and about 8 μM, between about 250 nM and about 7 μM, between about 250 nM and about 6 μM, between about 250 nM and about 5 μM, between about 250 nM and about 4 μM, between about 250 nM and about 3 μM, between about 250 nM and about 2 μM, between about 250 nM and about 1 μM, between about 250 nM and about 950 nM, between about 250 nM and about 900 nM, between about 250 nM and about 850 nM, between about 250 nM and about 800 nM, between about 250 nM and about 750 nM, between about 250 nM and about 700 nM, between about 250 nM and about 650 nM, between about 250 nM and about 600 nM, between about 250 nM and about 550 nM, between about 250 nM and about 500 nM, between about 250 nM and about 450 nM, between about 250 nM and about 400 nM, between about 250 nM and about 350 nM, between about 250 nM and about 300 nM, between about 500 nM and about 10 μM, between about 500 nM and about 9 μM, between about 500 nM and about 8 μM, between about 500 nM and about 7 μM, between about 500 nM and about 6 μM, between about 500 nM and about 5 μM, between about 500 nM and about 4 μM, between about 500 nM and about 3 μM, between about 500 nM and about 2 μM, between about 500 nM and about 1 μM, between about 500 nM and about 950 nM, between about 500 nM and about 900 nM, between about 500 nM and about 850 nM, between about 500 nM and about 800 nM, between about 500 nM and about 750 nM, between about 500 nM and about 700 nM, between about 500 nM and about 650 nM, between about 500 nM and about 600 nM, between about 500 nM and about 550 nM, between about 750 nM and about 10 μM, between about 750 nM and about 9 μM, between about 750 nM and about 8 μM, between about 750 nM and about 7 μM, between about 750 nM and about 6 μM, between about 750 nM and about 5 μM, between about 750 nM and about 4 μM, between about 750 nM and about 3 μM, between about 750 nM and about 2 μM, between about 750 nM and about 1 μM, between about 750 nM and about 950 nM, between about 750 nM and about 900 nM, between about 750 nM and about 850 nM, between about 750 nM and about 800 nM, between about 950 nM and about 10 μM, between about 950 nM and about 9 μM, between about 950 nM and about 8 μM, between about 950 nM and about 7 μM, between about 950 nM and about 6 μM, between about 950 nM and about 5 μM, between about 950 nM and about 4 μM, between about 950 nM and about 3 μM, between about 950 nM and about 2 μM, between about 950 nM and about 1 μM, between about 1 μM and about 10 μM, between about 1 μM and about 9 μM, between about 1 μM and about 8 μM, between about 1 μM and about 7 μM, between about 1 μM and about 6 μM, between about 1 μM and about 5 μM, between about 1 μM and about 4 μM, between about 1 μM and about 3 μM, between about 1 μM and about 2 μM, between about 2 μM and about 10 μM, between about 2 μM and about 9 μM, between bout 2 μM and about 8 μM, between about 2 μM and about 7 μM, between about 2 μM and about 6 μM, between about 2 μM and about 5 μM, between about 2 μM and about 4 μM, between about 2 μM and about 3 μM, between about 4 μM and about 10 μM, between about 4 μM and about 9 μM, between about 4 μM and about 8 μM, between about 4 μM and about 7 μM, between about 4 μM and about 6 μM, between about 4 μM and about 5 μM, between about 5 μM and about 10 μM, between about 5 μM and about 9 μM, between about 5 μM and about 8 μM, between about 5 μM and about 7 μM, between about 5 μM and about 6 μM, between about 6 μM and about 10 μM, between about 6 μM and about 9 μM, between about 6 μM and about 8 μM, between about 6 μM and about 7 μM; between about 7 μM and about 10 μM, between about 7 μM and about 9 μM, between about 7 μM and about 8 μM, between about 8 μM and about 10 μM, between about 8 μM and about 9 μM, or between about 9 μM and about 10 μM) for one or more of DYRK1A, DYRK1B, DYRK2, DYRK3, and DYRK 4. In some embodiments, the DYRK inhibitor has an IC₅₀ of between about 100 pM and about 10 μM (or any of the subranges of this range described herein) for each of DYRK1A, DYRK1B, DYRK2, DYRK3 and DYRK4. In some embodiments, the DYRK inhibitor has an IC₅₀ of between about 100 pM and about 10 μM (or any of the subranges of this range) for each of DYRK1A and DYRK1B. In some embodiments, the DYRK inhibitor has an IC₅₀ of between about 100 pM and about 10 μM (or any of the subranges of this range described herein) for each of DYRK1A and DYRK2. In some embodiments, the DYRK inhibitor has an IC₅₀ of between about 100 pM and about 10 μM (or any of the subranges of this range described herein) for each of DYRK1A and DYRK3. In some embodiments, the DYRK inhibitor has an IC₅₀ of between about 100 pM and about 10 μM (or any of the subranges of this range described herein) for each of DYRK1A and DYRK4. In some embodiments, the DYRK inhibitor has an IC₅₀ of between about 100 pM and about 10 μM (or any of the subranges of this range described herein) for each of DYRK1B and DYRK2. In some embodiments, the DYRK inhibitor has an IC₅₀ of between about 100 pM and about 10 μM (or any of the subranges of this range described herein) for each of DYRK1B and DYRK3. In some embodiments, the DYRK inhibitor has an IC₅₀ of between about 100 pM and about 10 μM (or any of the subranges of this range described herein) for each of DYRK1B and DYRK4. In some embodiments, the DYRK inhibitor has an IC₅₀ of between about 100 pM and about 10 μM (or any of the subranges of this range described herein) for each of DYRK2 and DYRK3. In some embodiments, the DYRK inhibitor has an IC₅₀ of between about 100 pM and about 10 μM (or any of the subranges of this range described herein) for each of DYRK2 and DYRK4. In some embodiments, the DYRK inhibitor has an IC₅₀ of between about 100 pM and about 10 μM (or any of the subranges of this range described herein) for each of DYRK3 and DYRK4. In some embodiments, the DYRK inhibitor has an IC₅₀ of between about 100 pM and about 10 μM (or any of the subranges of this range described herein) for each of DYRK1A, DYRK2, DYRK3, and DYRK4. In some embodiments, the DYRK inhibitor has an IC₅₀ of between about 100 pM and about 10 μM (or any of the subranges of this range described herein) for each of DYRK1A, DYRK1B, DYRK3, and DYRK4. In some embodiments, the DYRK inhibitor has an IC₅₀ of between about 100 pM and about 10 μM (or any of the subranges of this range described herein) for each of DYRK1A, DYRK1B, DYRK2, and DYRK4. In some embodiments, the DYRK inhibitor has an IC₅₀ of between about 100 pM and about 10 μM (or any of the subranges of this range described herein) for each of DYRK1A, DYRK1B, DYRK2, and DYRK3. In some embodiments, the DYRK inhibitor has an IC₅₀ of between about 100 pM and about 10 μM (or any of the subranges of this range described herein) for each of DYRK1B, DYRK2, DYRK3, and DYRK4. In some embodiments, the DYRK inhibitor has an IC₅₀ of between about 100 pM and about 10 μM (or any of the subranges of this range described herein) for each of DYRK1A, DYRK3, and DYRK4. In some embodiments, the DYRK inhibitor has an IC₅₀ of between about 100 pM and about 10 μM (or any of the subranges of this range described herein) for each of DYRK1A, DYRK2, and DYRK4. In some embodiments, the DYRK inhibitor has an IC₅₀ of between about 100 pM and about 10 μM (or any of the subranges of this range described herein) for each of DYRK1A, DYRK1B, and DYRK4. In some embodiments, the DYRK inhibitor has an IC₅₀ of between about 100 pM and about 10 μM (or any of the subranges of this range described herein) for each of DYRK1B, DYRK2, and DYRK3. In some embodiments, the DYRK inhibitor has an IC₅₀ of between about 100 pM and about 10 μM (or any of the subranges of this range described herein) for each of DYRK1B, DYRK2, and DYRK4. In some embodiments, the DYRK inhibitor has an IC₅₀ of between about 100 pM and about 10 μM (or any of the subranges of this range described herein) for each of DYRK1A, DYRK3, and DYRK4. In some embodiments, the DYRK inhibitor has an IC₅₀ of between about 100 pM and about 10 μM (or any of the subranges of this range described herein) for each of DYRK2, DYRK3, and DYRK4. In some embodiments, the DYRK inhibitor has an IC₅₀ of between about 100 pM and about 10 μM (or any of the subranges of this range described herein) for each of DYRK1A and DYRK1B. In some embodiments, the DYRK inhibitor has an IC₅₀ of between about 100 pM and about 10 μM (or any of the subranges of this range described herein) for each of DYRK1A and DYRK2. In some embodiments, the DYRK inhibitor has an IC₅₀ of between about 100 pM and about 10 μM (or any of the subranges of this range described herein) for each of DYRK1A and DYRK3. In some embodiments, the DYRK inhibitor has an IC₅₀ of between about 100 pM and about 10 μM (or any of the subranges of this range described herein) for each of DYRK1A and DYRK4. In some embodiments, the DYRK inhibitor has an IC₅₀ of between about 100 pM and about 10 μM (or any of the subranges of this range described herein) for each of DYRK1B and DYRK2. In some embodiments, the DYRK inhibitor has an IC₅₀ of between about 100 pM and about 10 μM (or any of the subranges of this range described herein) for each of DYRK1B and DYRK3. In some embodiments, the DYRK inhibitor has an IC₅₀ of between about 100 pM and about 10 μM (or any of the subranges of this range described herein) for each of DYRK1B, and DYRK4. In some embodiments, the DYRK inhibitor has an IC₅₀ of between about 100 pM and about 10 μM (or any of the subranges of this range described herein) for each of DYRK2, and DYRK3. In some embodiments, the DYRK inhibitor has an IC₅₀ of between about 100 pM and about 10 μM (or any of the subranges of this range described herein) for each of DYRK2, and DYRK4. In some embodiments, the DYRK inhibitor has an IC₅₀ of between about 100 pM and about 10 μM (or any of the subranges of this range described herein) for each of DYRK3, and DYRK4.

In some embodiments, the DYRK inhibitor is a compound of Formula (I), or a pharmaceutically acceptable salt or solvate thereof.

In some embodiments, the DYRK inhibitor is a compound of Formula (II), or a pharmaceutically acceptable salt or solvate thereof.

In some embodiments, the DYRK inhibitor is a compound of Formula (III), or a pharmaceutically acceptable salt or solvate thereof.

In some embodiments, the DYRK inhibitor is a compound of Formula (IV), or a pharmaceutically acceptable salt or solvate thereof.

In some embodiments, the DYRK inhibitor is a compound of Formula (V), or a pharmaceutically acceptable salt or solvate thereof.

In some embodiments, the DYRK inhibitor is a compound of Formula (VI), or a pharmaceutically acceptable salt or solvate thereof.

In some embodiments, the DYRK inhibitor is a compound of Formula (VII), or a pharmaceutically acceptable salt or solvate thereof.

In some embodiments, the DYRK inhibitor is a compound of Formula (VIII), or a pharmaceutically acceptable salt or solvate thereof.

### Exemplary Dual CLK/DYRK Inhibitors,

In some embodiments, the methods provided herein include a single inhibitor, wherein the single inhibitor is a dual CLK/DYRK inhibitor. In some embodiments, the methods provided herein include a single inhibitor, wherein the single inhibitor is a dual DYRK1A/CLK2 and/or CLK3 inhibitor. In some embodiments, the CLK inhibitor also acts as a DYRK inhibitor. In some embodiments, the DYRK inhibitor also acts as a CLK inhibitor.

In some embodiments, the dual CLK/DYRK inhibitor inhibits one or more of the DYRK family members (DYRK1A, DYRK1B, DYRK2, DYRK3, and DYRK4) and one or more of the CLK family members (CLK1, CLK2, CLK3, and CLK4). In some embodiments, the dual CLK/DYRK inhibitor is a broad spectrum inhibitor, inhibiting two or more of the DYRK family members DYRK1A, DYRK1B, DYRK2, DYRK3, and DYRK4; and one or more of the CLK family members. In other embodiments, the dual CLK/DYRK inhibitor is a broad spectrum inhibitor, inhibiting two or more of the CLK family members CLK1, CLK2, CLK3, and CLK4; and one or more of the DYRK family members. In still other embodiments, the dual CLK/DYRK inhibitor is a broad spectrum inhibitor, inhibiting two or more of the CLK family members; and two or more of the DYRK family members.

In some embodiments, the dual CLK/DYRK inhibitor is a dual DYRK1A/CLK2 and/or CLK3 inhibitor. In some embodiments, the dual CLK/DYRK inhibitor inhibits DYKR1A and CLK2. In other embodiments, the dual CLK/DYRK inhibitor inhibits DYKR1A and CLK3. In still other embodiments, the dual CLK/DYRK inhibitor inhibits DYKR1A and both CLK2 and CLK3.

In some embodiments, a single inhibitor has an IC₅₀ of between about 100 pM and about 10 μM (or any of the subranges of this range described herein) for any combination of CLK and DYRK family members. In non-limiting examples, the single inhibitor has an IC₅₀ of between about 100 pM and about 10 μM (or any of the subranges of this range described herein) for CLK1 with one or more CLK or DYRK family members (e.g. CLK2, CLK3, CLK4, DYRK1A, DYRK1B, DYRK2, DYRK3, DYRK4). In non-limiting examples, the single inhibitor has an IC₅₀ of between about 100 pM and about 10 μM (or any of the subranges of this range described herein) for CLK2 with one or more CLK or DYRK family members (e.g. CLK1, CLK3, CLK4, DYRK1A, DYRK1B, DYRK2, DYRK3, DYRK4). In non-limiting examples, the single inhibitor has an IC₅₀ of between about 100 pM and about 10 μM (or any of the subranges of this range described herein) for CLK3 with one or more CLK or DYRK family members (e.g. CLK1, CLK2, CLK4, DYRK1A, DYR1B, DYRK2, DYRK3, DYRK4). In non-limiting examples, the single inhibitor has an IC₅₀ of between about 100 pM and about 10 μM (or any of the subranges of this range described herein) for CLK4 with one or more CLK or DYRK family members (e.g. CLK1, CLK2, CLK3, DYRK1A, DYR1B, DYRK2, DYRK3, DYRK4). In non-limiting examples, the single inhibitor has an IC₅₀ of between about 100 pM and about 10 μM (or any of the subranges of this range described herein) for DYRK1A with one or more CLK or DYRK family members (e.g. CLK1, CLK2, CLK3, CLK4, DYR1B, DYRK2, DYRK3, DYRK4). In non-limiting examples, the single inhibitor has an IC₅₀ of between about 100 pM and about 10 μM (or any of the subranges of this range described herein) for DYRK1B with one or more CLK or DYRK family members (e.g. CLK1, CLK2, CLK3, CLK4, DYR1A, DYRK2, DYRK3, DYRK4). In non-limiting examples, the single inhibitor has an IC₅₀ of between about 100 pM and about 10 μM (or any of the subranges of this range described herein) for DYRK2 with one or more CLK or DYRK family members (e.g. CLK1, CLK2, CLK3, CLK4, DYR1A, DYRK1B, DYRK3, DYRK4). In non-limiting examples, the single inhibitor has an IC₅₀ of between about 100 pM and about 10 μM (or any of the subranges of this range described herein) for DYRK3 with one or more CLK or DYRK family members (e.g. CLK1, CLK2, CLK3, CLK4, DYR1A, DYRK1B, DYRK2, DYRK4). In non-limiting examples, the single inhibitor has an IC₅₀ of between about 100 pM and about 10 μM (or any of the subranges of this range described herein) for DYRK4 with one or more CLK or DYRK family members (e.g. CLK1, CLK2, CLK3, CLK4, DYR1A, DYRK1B, DYRK2, DYRK3).

In some embodiments, the dual CLK/DYRK inhibitor is a compound of Formula (I), or a pharmaceutically acceptable salt or solvate thereof.

In some embodiments, the dual CLK/DYRK inhibitor is a compound of Formula (II), or a pharmaceutically acceptable salt or solvate thereof.

In some embodiments, the dual CLK/DYRK inhibitor is a compound of Formula (III), or a pharmaceutically acceptable salt or solvate thereof.

In some embodiments, the dual CLK/DYRK inhibitor is a compound of Formula (IV), or a pharmaceutically acceptable salt or solvate thereof.

In some embodiments, the dual CLK/DYRK inhibitor is a compound of Formula (V), or a pharmaceutically acceptable salt or solvate thereof.

In some embodiments, the dual CLK/DYRK inhibitor is a compound of Formula (VI), or a pharmaceutically acceptable salt or solvate thereof.

In some embodiments, the dual CLK/DYRK inhibitor is a compound of Formula (VII), or a pharmaceutically acceptable salt or solvate thereof.

In some embodiments, the dual CLK/DYRK inhibitor is a compound of Formula (VIII), or a pharmaceutically acceptable salt or solvate thereof.

### Exemplary Compounds of Formulas (I)-(VIII)

U.S. Provisional Application No. 62/793,428 describes compounds having Formula (I) and is hereby incorporated by reference in its entirety.

Some embodiments of the present disclosure include compounds of Formula (I):

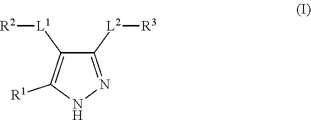

or pharmaceutically acceptable salts or solvates thereof.

In some embodiments of Formula (I), R¹ is selected from the group consisting of H, halide (e.g., F, Cl, Br, I), and unsubstituted —(C₁₋₃ alkyl).

In some embodiments of Formula (I), R² is selected from the group consisting of unsubstituted —(C₁₋₉ alkyl), unsubstituted —(C₂₋₉ alkenyl), unsubstituted —(C₁₋₉ haloalkyl), —(C₁₋₂ alkylene)ₚ(C₃₋₆ carbocyclyl) optionally substituted with 1-12 (e.g., 1-11, 1-10, 1-9, 1-8, 1-7, 1-6, 1-5, 1-4, 1-3, 1-2, 1) R⁴, -monocyclic heterocyclyl optionally substituted with 1-10 (e.g., 1-9, 1-8, 1-7, 1-6, 1-5, 1-4, 1-3, 1-2, 1) R⁵, -phenyl optionally substituted with 1-5 (e.g., 1-4, 1-3, 1-2, 1) R⁶, -heteroaryl optionally substituted with 1-4 (e.g., 1-3, 1-2, 1) R⁷, —CO₂R⁸, —OR⁹, and —(C═O)R¹⁰; wherein —(C₁₋₄ alkylene) is optionally substituted with one or more substituents as defined anywhere herein.

In some embodiments Formula (I), when R² is -heteroaryl optionally substituted with 1-4 R⁷, the heteroaryl is selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein

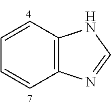

is only substituted at positions 4 and 7.

In some embodiments of Formula (I), R³ is selected from the group consisting of -heterocyclyl optionally substituted with 1-10 (e.g., 1-9, 1-8, 1-7, 1-6, 1-5, 1-4, 1-3, 1-2, 1) R¹, —(C₁₋₄ alkylene)ₚphenyl optionally substituted with 1-5 (e.g., 1-4, 1-3, 1-2, 1) R¹², -heteroaryl optionally substituted with 1-4 (e.g., 1-3, 1-2, 1) R¹³, and —(C₁₋₄ alkylene)OR¹⁴; wherein each —(C₁₋₄ alkylene) is, independently, optionally substituted with one or more substituents as defined anywhere herein.

In some embodiments Formula (I), when L² is a bond; R³ is selected from -heteroaryl optionally substituted with 1-4 R¹³; wherein heteroaryl selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein

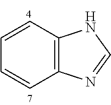

is only substituted at positions 4 and 7.

In some embodiments of Formula (I), each R⁴ is halide.

In some embodiments of Formula (I), each R⁵ is independently selected from the group consisting of halide and unsubstituted —(C₁₋₉ alkyl).

In some embodiments of Formula (I), each R⁶ is independently selected from the group consisting of unsubstituted —(C₁₋₉ alkyl), unsubstituted —(C₁₋₉ haloalkyl), —OR⁵, and —(C₁₋₄ alkylene)ₚN(R¹⁶)₂; wherein —(C₁₋₄ alkylene) is optionally substituted with one or more substituents as defined anywhere herein.

In some embodiments of Formula (I), each R⁷ is independently selected from the group consisting of halide (e.g., F, Cl, Br, I), unsubstituted —(C₁₋₉ alkyl), unsubstituted —(C₁₋₉ haloalkyl), —OR⁵, —CO₂R⁷, —NR¹⁸(C═O)R¹⁹, —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰, and —(C₁₋₄ alkylene)ₚN(R¹⁶)₂; wherein each —(C₁₋₄ alkylene) is, independently, optionally substituted with one or more substituents as defined anywhere herein.

In some embodiments of Formula (I), R⁸ is unsubstituted —(C₁₋₉ alkyl).

In some embodiments of Formula (I), R⁹ is unsubstituted —(C₁₋₉ alkyl).

In some embodiments of Formula (I), R¹⁰ is -aryl optionally substituted with 1-5 (e.g., 1-4, 1-3, 1-2, 1) R²¹.

In some embodiments of Formula (I), each R¹¹ is independently selected from the group consisting of halide and unsubstituted —(C₁₋₉ alkyl).

In some embodiments of Formula (I), each R¹² is independently selected from the group consisting of —(C₁₋₄alkylene)ₚheterocyclyl optionally substituted with 1-10 (e.g., 1-9, 1-8, 1-7, 1-6, 1-5, 1-4, 1-3, 1-2, 1) R²⁰, -aryl optionally substituted with 1-5 (e.g., 1-4, 1-3, 1-2, 1) R²², —(C₁₋₄ alkylene)N(R¹⁶)₂, and —OR²³; wherein each —(C₁₋₄ alkylene) is, independently, optionally substituted with one or more substituents as defined anywhere herein.

In some embodiments of Formula (I), each R¹³ is independently selected from the group consisting of halide (e.g., F, Cl, Br, I), unsubstituted —(C₁₋₉ alkyl), unsubstituted —(C₁₋₉ haloalkyl), —(C₁₋₄ alkylene)ₚN(R¹⁶)₂, —OR²³, —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰, -aryl optionally substituted with 1-5 (e.g., 1-4, 1-3, 1-2, 1) R²², and -heteroaryl optionally substituted with 1-4 (e.g., 1-3, 1-2, 1) R²⁴; wherein —(C₁₋₄ alkylene) is optionally substituted with one or more substituents as defined anywhere herein.

In some embodiments of Formula (I), R¹⁴ is selected from the group consisting of unsubstituted —(C₁₋₄ alkyl) and -aryl optionally substituted with 1-5 (e.g., 1-4, 1-3, 1-2, 1) R²².

In some embodiments of Formula (I), each R¹⁵ is independently selected from the group consisting of unsubstituted —(C₁₋₉ alkyl) and -heterocyclyl optionally substituted with 1-10 (e.g., 1-9, 1-8, 1-7, 1-6, 1-5, 1-4, 1-3, 1-2, 1) R²⁰.

In some embodiments of Formula (I), each R¹⁶ is independently selected from the group consisting of H and unsubstituted —(C₁₋₉ alkyl).

In some embodiments of Formula (I), each R¹⁷ is unsubstituted —(C₁₋₉ alkyl).

In some embodiments of Formula (I), each R¹⁸ is independently selected from the group consisting of H and unsubstituted —(C₁₋₉ alkyl).

In some embodiments of Formula (I), each R¹⁹ is unsubstituted —(C₁₋₉ alkyl).

In some embodiments of Formula (I), each R²⁰ is independently selected from the group consisting of halide and unsubstituted —(C₁₋₉ alkyl).

In some embodiments of Formula (I), each R²¹ is independently selected from the group consisting of halide and unsubstituted —(C₁₋₉ alkyl).

In some embodiments of Formula (I), each R²² is independently selected from the group consisting of halide and unsubstituted —(C₁₋₉ alkyl).

In some embodiments of Formula (I), each R²³ is independently selected from the group consisting of unsubstituted —(C₁₋₉ alkyl), —(C₁₋₄ alkylene)OR²⁵, and —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰; wherein each —(C₁₋₄ alkylene) is, independently, optionally substituted with one or more substituents as defined anywhere herein.

In some embodiments of Formula (I), each R²⁴ is independently selected from the group consisting of halide and unsubstituted —(C₁₋₉ alkyl).

In some embodiments of Formula (I), each R²⁵ is independently selected from the group consisting of H and unsubstituted —(C₁₋₉ alkyl).

In some embodiments of Formula (I), L¹ is selected from the group consisting of a bond, —CH═CH—, —C≡C—, —(CH₂)ₚNR¹⁸(C═O)—, —(C═O)NR¹⁸(CH₂)ₚ—, —NR¹⁸(C═O)NR¹⁸—, —NH(CH₂)ₚ—, and —(CH₂)ₚNH—.

In some embodiments of Formula (I), L² is selected from the group consisting of a bond, —(C═O)NR¹⁸—, —NR¹⁸(C═O)—, —NHCH₂—, and —CH₂NH—.

In some embodiments of Formula (I), each p is independently an integer of 0 or 1.

In some embodiments of Formula (I), or pharmaceutically acceptable salts or solvates thereof:

R¹ is H or methyl;

R² is -monocyclic heterocyclyl optionally substituted with 1-2 R⁵; or pyridinyl, pyrimidinyl, pyrazinyl, benzimidazolyl, each optionally substituted with 1-2 R⁷;

R³ is phenyl optionally substituted with 1-2 R¹²; or

R³ is pyridinyl, pyrimidinyl, pyrazinyl, pyridazinyl, imidazo[4,5-b]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, or imidazo[4,5-c]pyridinyl, each optionally substituted with 1-2 R¹³;

each R⁵ is independently selected from the group consisting of F, methyl, and ethyl;

each R⁷ is independently selected from the group consisting of F, methyl, —CH₂F, —CHF₂, —CF₃, and —OR¹⁵;

each R¹² is independently —(C₁ alkylene)ₚheterocyclyl optionally substituted with 1-2 R²⁰, phenyl optionally substituted with 1-2 R²², and —OR²³; wherein each heterocyclyl selected from the group consisting of azetidinyl, pyrrolidinyl, piperidinyl, and piperazinyl;

each R¹³ is independently F, methyl, —CH₂F, —CHF₂, —CF₃, —OR²³, -heterocyclyl optionally substituted with 1-2 R²⁰, and -phenyl optionally substituted with 1-2 R²²;

each R¹⁵ is independently selected from the group consisting of methyl and -unsubstituted monocyclic heterocyclyl;

each R²⁰ is F or methyl;
each R²² is F or methyl;

each R²³ is independently —(C₁₋₄ alkylene)ₚheterocyclyl optionally substituted with 1-2 F;

L¹ is CH═CH—, —C≡C—, or —NH(C═O)—; and

L² is —(C═O)NH—.

Bioorganic & Medicinal Chemistry Letters (2006), 16(14), 3740-3744 and U.S. application Ser. Nos. 10/295,833 and 10/317,914 describe compounds having Formula (II) and are each hereby incorporated by reference in their entirety.

Some embodiments of the present disclosure include compounds of Formula (II):

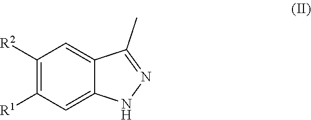

or pharmaceutically acceptable salts or solvates thereof.

In some embodiments of Formula (II), R¹ is selected from the group consisting of H and halide.

In some embodiments of Formula (II), R² is a 6-membered -heteroaryl optionally substituted with 1-4 (e.g., 1-3, 1-2, 1) R³.

In some embodiments of Formula (II), each R³ is selected from the group consisting of —OR⁴, —NHR⁵, and —(C₁₋₄ alkylene)ₚheterocyclyl optionally substituted with 1-10 (e.g., 1-9, 1-8, 1-7, 1-6, 1-5, 1-4, 1-3, 1-2, 1) R⁶; wherein —(C₁₋₄ alkylene) is optionally substituted with one or more substituents as defined anywhere herein.

In some embodiments of Formula (II), each R⁴ is independently selected from the group consisting of -heterocyclyl optionally substituted with 1-10 (e.g., 1-9, 1-8, 1-7, 1-6, 1-5, 1-4, 1-3, 1-2, 1) R⁷ and —CH₂CH(R⁸)NH₂.

In some embodiments of Formula (II), each R⁵ is independently selected from the group consisting of —(C₁₋₄alkylene)ₚheterocyclyl optionally substituted with 1-10 (e.g., 1-9, 1-8, 1-7, 1-6, 1-5, 1-4, 1-3, 1-2, 1) R⁹ and -carbocyclyl optionally substituted with 1-12 (e.g., 1-11, 1-10, 1-9, 1-8, 1-7, 1-6, 1-5, 1-4, 1-3, 1-2, 1) R¹⁰; wherein —(C₁₋₄ alkylene) is optionally substituted with one or more substituents as defined anywhere herein.

In some embodiments of Formula (II), each R⁶ is independently selected from the group consisting of halide (e.g., F, Cl, Br, I), —NH₂, —OH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (II), each R⁷ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (II), each R⁸ is independently selected from the group consisting of —(C₁₋₄ alkylene)aryl optionally substituted with 1-5 (e.g., 1-4, 1-3, 1-2, 1) R¹¹ and —(C₁₋₄ alkylene)heteroaryl optionally substituted with 1-4 (e.g., 1-3, 1-2, 1) R¹²; wherein each —(C₁₋₄ alkylene) is, independently, optionally substituted with one or more substituents as defined anywhere herein.

In some embodiments of Formula (II), each R⁹ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (II), each R¹⁰ is independently selected from the group consisting of halide (e.g., F, Cl, Br, I), —OH, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (II), each R¹¹ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (II), each R¹² is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (II), each p is independently 0 or 1.

In some embodiments of compounds of Formula (II):

R¹ is H or F;

R² is selected from the group consisting of pyridinyl, pyrimidinyl, pyrazinyl, and pyridazinyl, each optionally substituted with R³;

R³ is selected from the group consisting of —OR⁴, —NHR⁵, and —(C₁ alkylene)heterocyclyl optionally substituted with 1-2 R⁶, and —(C₁ alkylene)heterocyclyl optionally substituted with 1-2 R⁶;

each R⁴ is independently selected from the group consisting of -heterocyclyl optionally substituted with 1-2 R⁷ and —CH₂CH(R⁸)NH₂;

each R⁵ is independently selected from the group consisting of -heterocyclyl optionally substituted with 1-2 R⁹;

each R⁶ is independently selected from the group consisting of F, —NH₂, —OH, and methyl;

each R⁷ is independently selected from the group consisting of F, methyl and ethyl;

each R⁸ is benzyl optionally substituted with 1-2 R¹¹;

each R⁹ is independently selected from the group consisting of F, methyl, and ethyl; and

each R¹¹ is independently selected from the group consisting of F, methyl, and —CF₃.

U.S. Provisional Application No. 62/634,656 and U.S. Pat. Nos. 9,221,793 and 9,745,271 describe compounds having Formula (III) and are each hereby incorporated by reference in their entirety.

Some embodiments of the present disclosure include compounds of Formula (III):

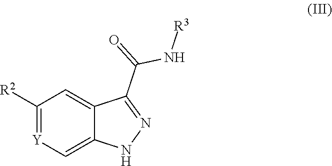

or pharmaceutically acceptable salts or solvates thereof.

In some embodiments of Formula (III), R¹ is selected from the group consisting of H, halide (e.g., F, Cl, Br, I), and methyl.

In some embodiments of Formula (III), R² is a -heteroaryl optionally substituted with 1-4 (e.g., 1-3, 1-2, 1) R⁴.

In some embodiments of Formula (III), R³ is selected from the group consisting of H, -aryl optionally substituted with 1-5 (e.g., 1-4, 1-3, 1-2, 1) R⁵; -heteroaryl optionally substituted with 1-4 (e.g., 1-3, 1-2, 1) R⁶, —C₁₋₆ alkyl optionally substituted with (i) phenyl optionally substituted with 1-5 (e.g., 1-4, 1-3, 1-2, 1) R¹¹ or (ii) —OR¹⁵, and carbocyclyl optionally substituted with phenyl.

In some embodiments of Formula (III), each R⁴ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚN(R⁷)(R⁸), —NHC(═O)R⁹, —(C₁₋₄ alkylene)ₚOR¹⁰, unsubstituted -carbocyclyl, —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁴, —(C₁₋₄ alkylene)ₚaryl optionally substituted with 1-5 (e.g., 1-4, 1-3, 1-2, 1) R¹¹, and —(C₁₋₄ alkylene)ₚheteroaryl optionally substituted with 1-4 (e.g., 1-3, 1-2, 1) R¹²; wherein each —(C₁₋₄ alkylene) is, independently, optionally substituted with one or more substituents as defined anywhere herein.

In some embodiments of Formula (III), each R⁵ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 (e.g., 1-4, 1-3, 1-2, 1) R¹³, —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein.

In some embodiments of Formula (III), each R⁶ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 (e.g., 1-4, 1-3, 1-2, 1) R¹³, —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein.

In some embodiments of Formula (III), each R⁷ is independently selected from the group consisting of H, unsubstituted —(C₁₋₆ alkyl), unsubstituted —(C₂₋₆ alkenyl), and unsubstituted —(C₂₋₆ alkynyl).

In some embodiments of Formula (III), each R⁸ is independently selected from the group consisting of H, unsubstituted —(C₁₋₆ alkyl), unsubstituted —(C₂₋₆ alkenyl), unsubstituted —(C₂₋₆ alkynyl), and -heterocyclyl optionally substituted with 1-10 (e.g., 1-9, 1-8, 1-7, 1-6, 1-5, 1-4, 1-3, 1-2, 1) R²¹.

In some embodiments of Formula (III), R⁷ and R⁸ are taken together to form a -heterocyclyl ring optionally substituted with 1-10 (e.g., 1-9, 1-8, 1-7, 1-6, 1-5, 1-4, 1-3, 1-2, 1) R²¹.

In some embodiments of Formula (III), each R⁹ is independently selected from the group consisting of —N(R²²)₂, -carbocyclyl optionally substituted with 1-12 (e.g., 1-11, 1-10, 1-9, 1-8, 1-7, 1-6, 1-5, 1-4, 1-3, 1-2, 1) R²³, -heterocyclyl optionally substituted with 1-10 (e.g., 1-9, 1-8, 1-7, 1-6, 1-5, 1-4, 1-3, 1-2, 1) R²¹, and -aryl optionally substituted with 1-5 (e.g., 1-4, 1-3, 1-2, 1) R²⁴.

In some embodiments of Formula (III), each R¹⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 (e.g., 1-9, 1-8, 1-7, 1-6, 1-5, 1-4, 1-3, 1-2, 1) R²¹.

In some embodiments of Formula (III), each R¹¹ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (III), each R¹² is independently selected from the group consisting of halide (e.g., F, Cl, Br, I), —(C₁₋₄ alkylene)ₚOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein.

In some embodiments of Formula (III), each R¹³ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (III), each R¹⁴ is independently selected from the group consisting of halide (e.g., F, Cl, Br, I), —(C₁₋₄ alkylene)ₚOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein.

In some embodiments of Formula (III), each R¹⁵ is independently selected from the group consisting of H, unsubstituted —(C₁₋₆ alkyl), unsubstituted —(C₂₋₆ alkenyl), unsubstituted —(C₂₋₆ alkynyl), and -carbocyclyl optionally substituted with 1-12 (e.g., 1-11, 1-10, 1-9, 1-8, 1-7, 1-6, 1-5, 1-4, 1-3, 1-2, 1) R²³.

In some embodiments of Formula (III), two adjacent R¹⁵ are taken together to form a -heterocyclyl ring optionally substituted with 1-10 (e.g., 1-9, 1-8, 1-7, 1-6, 1-5, 1-4, 1-3, 1-2, 1) R²¹.

In some embodiments of Formula (III), each R¹⁶ is independently selected from the group consisting of unsubstituted —(C₁₋₆ alkyl), unsubstituted —(C₂₋₆ alkenyl), unsubstituted —(C₂₋₆ alkynyl), and -carbocyclyl optionally substituted with 1-12 (e.g., 1-11, 1-10, 1-9, 1-8, 1-7, 1-6, 1-5, 1-4, 1-3, 1-2, 1) R²³.

In some embodiments of Formula (III), each R¹⁷ is independently selected from the group consisting of H, unsubstituted —(C₁₋₆ alkyl), unsubstituted —(C₂₋₆ alkenyl), and unsubstituted —(C₂₋₆ alkynyl).

In some embodiments of Formula (III), each R¹⁸ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)NMe₂, and -heterocyclyl ring optionally substituted with 1-10 (e.g., 1-9, 1-8, 1-7, 1-6, 1-5, 1-4, 1-3, 1-2, 1) R²¹; wherein —(C₁₋₄ alkylene) is optionally substituted with one or more substituents as defined anywhere herein.

In some embodiments of Formula (III), each R¹⁹ is independently selected from the group consisting of unsubstituted —(C₁₋₆ alkyl), unsubstituted —(C₂₋₆ alkenyl), and unsubstituted —(C₂₋₆ alkynyl).

In some embodiments of Formula (III), each R²⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —CH(CH₂OH)₂, —(C₁₋₄ alkylene)ₚheterocyclyl ring optionally substituted with 1-10 (e.g., 1-9, 1-8, 1-7, 1-6, 1-5, 1-4, 1-3, 1-2, 1) R²¹, and -aryl optionally substituted with 1-5 (e.g., 1-4, 1-3, 1-2, 1) R²⁴; wherein —(C₁₋₄ alkylene) is optionally substituted with one or more substituents as defined anywhere herein.

In some embodiments of Formula (III), each R²¹ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (III), each R²² is independently selected from the group consisting of unsubstituted —(C₁₋₆ alkyl), unsubstituted —(C₂₋₆ alkenyl), and unsubstituted —(C₂₋₆ alkynyl).

In some embodiments of Formula (III), each R²³ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (III), each R²⁴ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (III), Y is selected from the group consisting of —C(R¹)═ and —N═.

In some embodiments of Formula (III), each p is independently 0 or 1.

In some embodiments of the compounds of Formula (III), or pharmaceutically acceptable salts or solvates thereof:

Y is —C(R¹)═, and R¹ is H;

R² is pyridinyl, pyrimidinyl, pyrazinyl, or pyrazolinyl, each optionally substituted with 1-2 R⁴;

R³ is selected from the group consisting of -phenyl optionally substituted with 1-2 R⁵ and -monocyclic heteroaryl optionally substituted with 1-2 R⁶;

each R⁴ is independently selected from the group consisting of F, methyl, —NH₂, —(C₁₋₄ alkylene)ₚOH, —NHC(═O)R⁹, -aryl optionally substituted with 1-2 R¹¹, -heteroaryl optionally substituted with 1-2 R¹², and unsubstituted -carbocyclyl;

each R⁵ is independently selected from the group consisting of F, unsubstituted —(C₁₋₆ alkyl), unsubstituted —(C₁₋₆ haloalkyl), —(C₁₋₄ alkylene)ₚaryl optionally substituted with 1-2 R¹³, —(C₁₋₄ alkylene)ₚheterocyclyl optionally substituted with 1-2 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) unsubstituted;

each R⁶ is independently selected from the group consisting of F, unsubstituted —(C₁₋₆ alkyl), unsubstituted —(C₁₋₆ haloalkyl), —(C₁₋₄ alkylene)ₚaryl optionally substituted with 1-2 R¹³, —(C₁₋₄ alkylene)ₚheterocyclyl optionally substituted with 1-2 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is unsubstituted;

each R⁹ is independently selected from the group consisting of —N(R²²)₂, -carbocyclyl optionally substituted with 1-2 R²³, -heterocyclyl optionally substituted with 1-2 R²¹, and -aryl optionally substituted with 1-2 R²⁴;

each R¹¹ is independently selected from the group consisting of F, unsubstituted —(C₁₋₆ alkyl), and unsubstituted —(C₁₋₆ haloalkyl);

each R¹² is independently selected from the group consisting of F, —(C₁₋₄ alkylene)ₚOH, unsubstituted —(C₁₋₆ alkyl), and unsubstituted —(C₁₋₆ haloalkyl); wherein —(C₁₋₄ alkylene) is unsubstituted;

each R¹³ is independently selected from the group consisting of F, unsubstituted —(C₁₋₆ alkyl), and unsubstituted —(C₁₋₆ haloalkyl);

each R¹⁴ is independently selected from the group consisting of F, —(C₁₋₄ alkylene)ₚOH, unsubstituted —(C₁₋₆ alkyl), and unsubstituted —(C₁₋₆ haloalkyl); wherein —(C₁₋₄ alkylene) is unsubstituted;

each R¹⁵ is independently selected from the group consisting of H, unsubstituted —(C₁₋₆ alkyl), and -carbocyclyl optionally substituted with 1-2 R²³;

each R¹⁶ is independently selected from the group consisting of unsubstituted —(C₁₋₆ alkyl) and -carbocyclyl optionally substituted with 1-2 R²³;

each R¹⁷ is independently selected from the group consisting of H, unsubstituted —(C₁₋₆ alkyl), and unsubstituted —(C₂₋₆ alkynyl);

each R¹⁸ is independently selected from the group consisting of H, unsubstituted —(C₁₋₆ alkyl), —(C₁₋₄ alkylene)N(CH₃)₂, and -heterocyclyl ring optionally substituted with 1-2 R²¹; wherein —(C₁₋₄ alkylene) is unsubstituted;

each R¹⁹ is independently unsubstituted —(C₁₋₆ alkyl);

each R²⁰ is independently selected from the group consisting of H, unsubstituted —(C₁₋₆ alkyl), unsubstituted —(C₁₋₆haloalkyl), —CH(CH₂OH)₂, —(C₁₋₄ alkylene)ₚheterocyclyl ring optionally substituted with 1-2 R²¹, and -phenyl optionally substituted with 1-2 R²⁴; wherein —(C₁₋₄ alkylene) is unsubstituted;

each R²¹ is independently selected from the group consisting of F, unsubstituted —(C₁₋₆ alkyl), and unsubstituted —(C₁₋₆ haloalkyl);

each R²² is independently unsubstituted —(C₁₋₆ alkyl);

each R²³ is independently selected from the group consisting of F, unsubstituted —(C₁₋₆ alkyl), and unsubstituted —(C₁₋₆ haloalkyl);

each R²⁴ is independently selected from the group consisting of F, unsubstituted —(C₁₋₆ alkyl), and unsubstituted —(C₁₋₆ haloalkyl); and

each p is independently 0 or 1.

U.S. application Ser. Nos. 15/749,910, 15/749,922, 15/749,923, 15/749,929, and 15/773,951 and U.S. Pat. Nos. 8,252,812, 8,450,340, 8,673,936, 8,883,822, 9,908,867, 9,475,807, 9,475,825, 9,493,487, 9,540,398, 9,546,185, 9,657,016, 9,738,638, and 9,758,531 describe compounds having Formula (IV) and are each hereby incorporated by reference in their entirety.

Some embodiments of the present disclosure include compounds of Formula (IV):

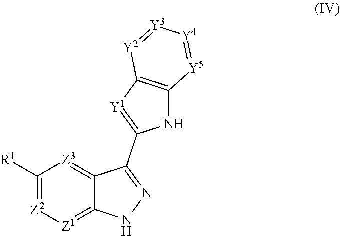

or pharmaceutically acceptable salts or solvates thereof.

In some embodiments of Formula (IV), R¹ is a -heteroaryl optionally substituted with 1-2 R³.

In some embodiments of Formula (IV), R² is selected from the group consisting of H, halide (e.g., F, Cl, Br, I), -aryl optionally substituted with 1-5 (e.g., 1-4, 1-3, 1-2, 1) R⁴-heteroaryl optionally substituted with 1-4 R⁵, and -heterocyclyl ring optionally substituted with 1-10 (e.g., 1-9, 1-8, 1-7, 1-6, 1-5, 1-4, 1-3, 1-2, 1) R⁶.

In some embodiments of Formula (IV), each R³ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R⁷, —C(═O)N(R⁸)₂, —NHC(═O)R⁹, —(C₁₋₄ alkylene)ₚN(R¹⁰)(R¹¹), —(C₁₋₄ alkylene)ₚOR¹², and -carbocyclyl optionally substituted with 1-12 (e.g., 1-11, 1-10, 1-9, 1-8, 1-7, 1-6, 1-5, 1-4, 1-3, 1-2, 1) R¹³; wherein each —(C₁₋₄ alkylene) is, independently, optionally substituted with one or more substituents as defined anywhere herein.

In some embodiments of Formula (IV), each R⁴ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚNHSO₂R¹⁴, —NR¹⁵(C₁₋₄ alkylene)NR¹⁵R¹⁶, —(C₁₋₄ alkylene)ₚNR¹⁵R¹⁶, —OR¹⁷, and -heterocyclyl optionally substituted with 1-10 (e.g., 1-9, 1-8, 1-7, 1-6, 1-5, 1-4, 1-3, 1-2, 1) R¹⁹; wherein each —(C₁₋₄ alkylene) is, independently, optionally substituted with one or more substituents as defined anywhere herein.

In some embodiments of Formula (IV), each R⁵ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), unsubstituted —(C₁₋₆ haloalkyl), and —C(═O)R¹⁸.

In some embodiments of Formula (IV), each R⁶ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (IV), each R⁷ is independently selected from the group consisting of halide (e.g., F, Cl, Br, I), —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (IV), each R⁸ is independently selected from the group consisting of H, unsubstituted —(C₁₋₆ alkyl), unsubstituted —(C₂₋₆ alkenyl), unsubstituted —(C₂₋₆ alkynyl), -heterocyclyl optionally substituted with 1-10 (e.g., 1-9, 1-8, 1-7, 1-6, 1-5, 1-4, 1-3, 1-2, 1) R¹⁹, —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; wherein —(C₁₋₄ alkylene) is optionally substituted with one or more substituents as defined anywhere herein.

In some embodiments of Formula (IV), each R⁹ is independently selected from the group consisting of unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁹, —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; —(C₁₋₄ alkylene)ₚaryl optionally substituted with 1-5 (e.g., 1-4, 1-3, 1-2, 1) R²¹, —(C₁₋₄ alkylene)ₚN(R²²)₂; wherein each —(C₁₋₄ alkylene) is, independently, optionally substituted with one or more substituents as defined anywhere herein.

In some embodiments of Formula (IV), each R¹⁰ is independently selected from the group consisting of H, unsubstituted —(C₁₋₆ alkyl), unsubstituted —(C₂₋₆ alkenyl), and unsubstituted —(C₂₋₆ alkynyl).

In some embodiments of Formula (IV), each R¹¹ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; and —(C₁₋₄ alkylene)ₚaryl optionally substituted with 1-5 (e.g., 1-4, 1-3, 1-2, 1) R²¹; wherein each —(C₁₋₄ alkylene) is, independently, optionally substituted with one or more substituents as defined anywhere herein.

In some embodiments of Formula (IV), each R¹² is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁹, —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; —(C₁₋₄ alkylene)ₚaryl optionally substituted with 1-5 (e.g., 1-4, 1-3, 1-2, 1) R²¹, —(C₁₋₄ alkylene)ₚN(R²²)₂; wherein each —(C₁₋₄ alkylene) is, independently, optionally substituted with one or more substituents as defined anywhere herein.

In some embodiments of Formula (IV), each R¹³ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (IV), each R¹⁴ is independently selected from the group consisting of unsubstituted —(C₁₋₆ alkyl), unsubstituted —(C₂₋₆ alkenyl), and unsubstituted —(C₂₋₆ alkynyl).

In some embodiments of Formula (IV), each R¹⁵ is independently selected from the group consisting of H, unsubstituted —(C₁₋₆ alkyl), unsubstituted —(C₂₋₆ alkenyl), and unsubstituted —(C₂₋₆ alkynyl).

In some embodiments of Formula (IV), each R¹⁶ is independently selected from the group consisting of H, unsubstituted —(C₁₋₆ alkyl), unsubstituted —(C₂₋₆ alkenyl), and unsubstituted —(C₂₋₆ alkynyl).

In some embodiments of Formula (IV), each R¹⁷ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁹, and, —(C₁₋₄ alkylene)ₚN(R²²)₂; wherein each —(C₁₋₄ alkylene) is, independently, optionally substituted with one or more substituents as defined anywhere herein.

In some embodiments of Formula (IV), each R¹⁸ is independently selected from the group consisting of unsubstituted —(C₁₋₆ alkyl), unsubstituted —(C₂₋₆ alkenyl), and unsubstituted —(C₂₋₆ alkynyl).

In some embodiments of Formula (IV), each R¹⁹ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (IV), each R²⁰ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (IV), each R²¹ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (IV), each R²² is independently selected from the group consisting of H, unsubstituted —(C₁₋₆ alkyl), unsubstituted —(C₂₋₆ alkenyl), and unsubstituted —(C₂₋₆ alkynyl).

In some embodiments of Formula (IV), each R²³ is independently selected from the group consisting of H and halide.

In some embodiments of Formula (IV), R²⁴ is selected from the group consisting of H, halide (e.g., F, Cl, Br, I), and —OR¹⁷.

In some embodiments of Formula (IV), Y¹ is selected from the group consisting of —CH═ and —N═.

In some embodiments of Formula (IV), Y² is selected from the group consisting of —C(R²)═ and —N═.

In some embodiments of Formula (IV), there is the proviso that when Y¹ is —N=then Y² is —C(R²)═.

In some embodiments of Formula (IV), Y³ is selected from the group consisting of —C(R²⁴)═ and —N═.

In some embodiments of Formula (IV), Y⁴ and Y⁵ are independently selected from the group consisting of —C(R²³)═ and —N═.

In some embodiments of Formula (IV), Z¹, Z², and Z³ are independently selected from the group consisting of —C(R²³)═ and —N═.

In some embodiments of Formula (IV), if Y² is nitrogen then Y³, Y⁴, and Y⁵ are carbon, and R² is absent.

In some embodiments of Formula (IV), if Y³ is nitrogen then Y⁴ and Y⁵ are carbon.

In some embodiments of Formula (IV), if Y⁴ is nitrogen then Y³ and Y⁵ are carbon.

In some embodiments of Formula (IV), if Y⁵ is nitrogen then Y³ and Y⁴ are carbon.

In some embodiments of Formula (IV), if Z¹ is nitrogen then Z² and Z³ are carbon.

In some embodiments of Formula (IV), if Z² is nitrogen then Z¹ and Z³ are carbon.

In some embodiments of Formula (IV), if Z³ is nitrogen then Z¹ and Z² are carbon.

In some embodiments of Formula (IV), each p is independently 0 or 1.

In some embodiments of the compounds of Formula (IV), or pharmaceutically acceptable salts or solvates thereof:

Y¹ is —N═;

Y² is —C(R²)═;

Y³, Y⁴, and Y⁵ are each —C(H)═;

Z¹, Z², and Z³ are independently selected from the group consisting of —C(H)═ and —N═;

R¹ is selected from the group consisting of pyridinyl, pyrimidinyl, pyridazinyl, pyrazinyl, pyrazolyl, and imidazolyl, each optionally substituted with 1 R³;

R² is selected from the group consisting of H, F, -phenyl optionally substituted with 1 R⁴-heteroaryl optionally substituted with 1 R⁵, and -heterocyclyl ring optionally substituted with 1 R⁶;

each R³ is independently selected from the group consisting of F, unsubstituted —(C₁₋₆ alkyl), unsubstituted —(C₁₋₆ haloalkyl), —(C₁₋₄ alkylene)ₚheterocyclyl optionally substituted with 1-2 R⁷, —C(═O)N(R⁸)₂, —NHC(═O)R⁹, —(C₁₋₄ alkylene)ₚN(R¹⁰)(R¹¹), —(C₁₋₄ alkylene)ₚOR¹², and -carbocyclyl optionally substituted with 1-2 R¹³; wherein each —(C₁₋₄ alkylene) is unsubstituted;

each R⁴ is independently selected from the group consisting of F, methyl, —CF₃, and —OR¹⁷;

each R⁵ is independently selected from the group consisting of F, methyl, and —CF₃;

each R⁶ is independently selected from the group consisting of F, methyl, and —CF₃;

each R⁷ is independently selected from the group consisting of F, —NH₂, and unsubstituted —(C₁₋₆ alkyl);

each R⁸ is independently selected from the group consisting of H, unsubstituted —(C₁₋₆ alkyl), -heterocyclyl optionally substituted with 1-2 R¹⁹, —(C₁₋₄ alkylene)ₚcarbocyclyl optionally substituted with 1-2 R²⁰; wherein —(C₁₋₄ alkylene) is unsubstituted;

each R⁹ is independently selected from the group consisting of unsubstituted —(C₁₋₆ alkyl), —(C₁₋₄ alkylene)ₚheterocyclyl optionally substituted with 1-2 R¹⁹, —(C₁₋₄ alkylene)ₚcarbocyclyl optionally substituted with 1-2 R²⁰; —(C₁₋₄ alkylene)ₚphenyl optionally substituted with 1-2 R²¹, —(C₁₋₄ alkylene)ₚN(R²²)₂; wherein each —(C₁₋₄ alkylene) is unsubstituted;

each R¹⁰ is independently selected from the group consisting of H, unsubstituted —(C₁₋₆ alkyl), unsubstituted —(C₂₋₆ alkenyl), and unsubstituted —(C₂₋₆ alkynyl);

each R¹¹ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)ₚcarbocyclyl optionally substituted with 1-12 R²⁰; and —(C₁₋₄ alkylene)ₚaryl optionally substituted with 1-5 (e.g., 1-4, 1-3, 1-2, 1) R²¹; wherein each —(C₁₋₄ alkylene) is unsubstituted;

each R¹² is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 (e.g., 1-4, 1-3, 1-2, 1) R²¹, —(C₁₋₄ alkylene)ₚN(R²²)₂; wherein each —(C₁₋₄ alkylene) is unsubstituted;

each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹⁷ is independently selected from the group consisting of H, unsubstituted —(C₁₋₆ alkyl), and unsubstituted —(C₁₋₄ alkylene)ₚheterocyclyl; wherein each —(C₁₋₄ alkylene) is unsubstituted;

each R¹⁹ is independently selected from the group consisting of F, unsubstituted —(C₁₋₆ alkyl), and unsubstituted —(C₁₋₆ haloalkyl);

each R²⁰ is independently selected from the group consisting of F, unsubstituted —(C₁₋₆ alkyl), and unsubstituted —(C₁₋₆ haloalkyl);

each R²¹ is independently selected from the group consisting of F, unsubstituted —(C₁₋₆ alkyl), and unsubstituted —(C₁₋₆ haloalkyl);

each R²² is independently selected from the group consisting of H and unsubstituted —(C₁₋₆ alkyl); and

and each p is independently 0 or 1.

U.S. Provisional Application Nos. 62/577,818, 62/578,370, 62/578,691, and 62/579,883, U.S. application Ser. Nos. 15/498,990 and 15/499,013, and U.S. Pat. No. 9,951,048 describe compounds having Formula (V) and are each hereby incorporated by reference in their entirety.

Some embodiments of the present disclosure include compounds of Formula (V):

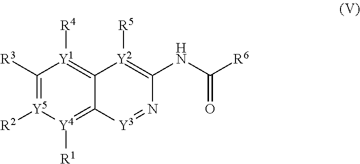

or pharmaceutically acceptable salts or solvates thereof.

In some embodiments of Formula (V), R¹, R², R⁴, and R⁵ are independently absent or selected from the group consisting of H, halide (e.g., F, Cl, Br, I), unsubstituted —(C₁₋₃ haloalkyl), and unsubstituted —(C₁₋₃ alkyl).

In some embodiments of Formula (V), R³ is selected from the group consisting of -aryl optionally substituted with 1-5 (e.g., 1-4, 1-3, 1-2, 1) R⁷ and -heteroaryl optionally substituted with 1-4 (e.g., 1-3, 1-2, 1) R⁸.

In some embodiments of Formula (V), R⁶ is selected from the group consisting of —(C₁₋₄ alkylene)ₚaryl optionally substituted with 1-5 (e.g., 1-4, 1-3, 1-2, 1) R⁹, —(C₂₋₄ alkenylene)ₚaryl optionally substituted with 1-5 (e.g., 1-4, 1-3, 1-2, 1) R⁹, —(C₁₋₄ alkylene)ₚheteroaryl optionally substituted with 1-6 R¹⁰; —(C₁₋₄ alkylene)ₚheterocyclyl optionally substituted with 1-10 (e.g., 1-9, 1-8, 1-7, 1-6, 1-5, 1-4, 1-3, 1-2, 1) R¹², —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R¹², —(C₁₋₄ alkylene)N(R¹³)(R¹⁴), —N(R¹⁵)(R¹⁶), —CF(C₁₋₉ alkyl)₂, —(C₁₋₄ alkylene)ₚO(C₃₋₉ alkyl), and —(C₂₋₉ alkynyl) optionally substituted with one or more halides; wherein each alkyl of —CF(C₁₋₉ alkyl)₂ is, independently, optionally substituted with one or more halides; wherein each —(C₁₋₄ alkylene) is, independently, optionally substituted with one or more substituents as defined anywhere herein; wherein —(C₁₋₄ alkenylene) is, optionally substituted with one or more substituents as defined anywhere herein.

In some embodiments of Formula (V), R⁷ is selected from the group consisting of halide and —N(R¹⁷)₂.

In some embodiments of Formula (V), each R⁸ is independently selected from the group consisting of H, halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —N(R¹⁵)(R¹⁸), —(C₁₋₄ alkylene)ₚXR¹⁹, —C(═O)N(R¹⁵)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰, and -carbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), two adjacent R⁸ are taken together to form a ring which is selected from the group consisting of -heterocyclyl optionally substituted with 1-10 (e.g., 1-9, 1-8, 1-7, 1-6, 1-5, 1-4, 1-3, 1-2, 1) R²² and -carbocyclyl optionally substituted with 1-12 (e.g., 1-11, 1-10, 1-9, 1-8, 1-7, 1-6, 1-5, 1-4, 1-3, 1-2, 1) R²¹.

In some embodiments of Formula (V), each R⁹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —XR²³, —C(═O)N(R¹⁵)₂, —(C₁₋₄ alkylene)ₚN(R²⁴)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R²², and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), each R¹⁰ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —XR²³, —C(═O)N(R¹⁵)₂, —(C₁₋₄ alkylene)ₚN(R²⁴)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R²², and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), each R¹¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚOR¹⁹, —(C₁₋₄ alkylene)ₚheterocyclyl optionally substituted with 1-10 (e.g., 1-9, 1-8, 1-7, 1-6, 1-5, 1-4, 1-3, 1-2, 1) R²², —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹, —N(R¹⁵)(R²⁵), —C(═O)(R²⁶), —(C₁₋₄ alkylene)C(═O)OR²⁷, —(C₁₋₄ alkylene)aryl optionally substituted with one or more halides, —(C₁₋₄ alkylene)ₚheteroaryl optionally substituted with one or more halides, and —SO₂(R²⁸); wherein each —(C₁₋₄ alkylene) is, independently, optionally substituted with one or more substituents as defined anywhere herein.

In some embodiments of Formula (V), two R¹¹ attached to the same carbon atom can together represent ═O to form a carbonyl group.

In some embodiments of Formula (V), each R¹² is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚOR¹⁹, —N(R¹⁵)(R²⁹), —C(═O)(R²⁶), —C(═O)OR²⁷, —(C₁₋₄ alkylene)ₚheterocyclyl optionally substituted with 1-10 (e.g., 1-9, 1-8, 1-7, 1-6, 1-5, 1-4, 1-3, 1-2, 1) R²², and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), R¹³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰, and -carbocyclyl optionally substituted with 1-12 (e.g., 1-11, 1-10, 1-9, 1-8, 1-7, 1-6, 1-5, 1-4, 1-3, 1-2, 1) R²¹; wherein —(C₁₋₄ alkylene) is, optionally substituted with one or more substituents as defined anywhere herein.

In some embodiments of Formula (V), R¹⁴ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰, and -carbocyclyl optionally substituted with 1-12 (e.g., 1-11, 1-10, 1-9, 1-8, 1-7, 1-6, 1-5, 1-4, 1-3, 1-2, 1) R²¹; wherein —(C₁₋₄ alkylene) is, optionally substituted with one or more substituents as defined anywhere herein.

In some embodiments of Formula (V), each R¹⁵ is selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl).

In some embodiments of Formula (V), R¹⁶ is selected from the group consisting of —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), R¹⁷ is independently selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl).

In some embodiments of Formula (V), two adjacent R¹⁷ are taken together to form a -heterocyclyl ring optionally substituted with 1-10 (e.g., 1-9, 1-8, 1-7, 1-6, 1-5, 1-4, 1-3, 1-2, 1) R²².

In some embodiments of Formula (V), R¹⁸ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C═O)R¹⁵, and —(C₁₋₄ alkylene)ₚheterocyclyl optionally substituted with one or more halides or one or more unsubstituted —(C₁₋₅ alkyl); wherein —(C₁₋₄ alkylene) is, independently, optionally substituted with one or more substituents as defined anywhere herein.

In some embodiments of Formula (V), each R¹⁹ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), each R²⁰ independently is selected from the group consisting of halide (e.g., F, Cl, Br, I), unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —N(R¹⁵)₂, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), each R²¹ is independently selected from the group consisting of halide (e.g., F, Cl, Br, I), unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN.

In some embodiments of Formula (V), each R²² is independently selected from the group consisting of halide (e.g., F, Cl, Br, I), unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —N(R¹⁵)₂, —C(═O)R³⁴, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), each R²³ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)N(R¹⁵)₂, —(C₁₋₄ alkylene)ₚaryl optionally substituted with 1-10 (e.g., 1-9, 1-8, 1-7, 1-6, 1-5, 1-4, 1-3, 1-2, 1) R³⁰, —(C₁₋₄ alkylene)ₚheterocyclyl optionally substituted with 1-12 (e.g., 1-11, 1-10, 1-9, 1-8, 1-7, 1-6, 1-5, 1-4, 1-3, 1-2, 1) R³¹, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), each R²⁴ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)N(R¹⁵)₂; wherein each —(C₁₋₄ alkylene) is, independently, optionally substituted with one or more substituents as defined anywhere herein.

In some embodiments of Formula (V), each R²⁵ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R³², —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹, —(C₁₋₄ alkylene)OR³³; wherein each —(C₁₋₄ alkylene) is, independently, optionally substituted with one or more substituents as defined anywhere herein.

In some embodiments of Formula (V), R²⁶ is selected from the group consisting of H, unsubstituted —(C₃₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or unsubstituted —(C₁₋₅ alkyl), —(C₁₋₄ alkylene)ₚheteroaryl optionally substituted with one or more halides or one or more unsubstituted —(C₁₋₅ alkyl), and —(C₁₋₄ alkylene)ₚheterocyclyl optionally substituted with one or more halides or one or more unsubstituted —(C₁₋₅ alkyl); wherein each —(C₁₋₄ alkylene) is, independently, optionally substituted with one or more substituents as defined anywhere herein.

In some embodiments of Formula (V), R²⁷ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or one or more unsubstituted —(C₁₋₅ alkyl), —(C₁₋₄ alkylene)ₚheteroaryl optionally substituted with one or more halides or unsubstituted —(C₁₋₅ alkyl), and —(C₁₋₄ alkylene)ₚheterocyclyl optionally substituted with one or more halides or one or more unsubstituted —(C₁₋₅ alkyl); wherein each —(C₁₋₄ alkylene) is, independently, optionally substituted with one or more substituents as defined anywhere herein.

In some embodiments of Formula (V), R²⁸ is selected from the group consisting of unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or one or more unsubstituted —(C₁₋₅ alkyl), —(C₁₋₄ alkylene)ₚheteroaryl optionally substituted with one or more halides or one or more unsubstituted —(C₁₋₅ alkyl), and —(C₁₋₄ alkylene)ₚheterocyclyl optionally substituted with one or more halides or one or more unsubstituted —(C₁₋₅ alkyl); wherein —(C₁₋₄ alkylene) is, optionally substituted with one or more substituents as defined anywhere herein.

In some embodiments of Formula (V), each R²⁹ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with 1-10 (e.g., 1-9, 1-8, 1-7, 1-6, 1-5, 1-4, 1-3, 1-2, 1) R³², —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹, —(C₁₋₄ alkylene)OR³³, and —C(═O)O(C₁₋₅ alkyl); wherein each —(C₁₋₄ alkylene) is, independently, optionally substituted with one or more substituents as defined anywhere herein.

In some embodiments of Formula (V), each R³⁰ is independently selected from the group consisting of halide (e.g., F, Cl, Br, I), unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN.

In some embodiments of Formula (V), each R³¹ is independently selected from the group consisting of halide (e.g., F, Cl, Br, I), unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —C(═O)R³⁴, —N(R²⁴)₂, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein.

In some embodiments of Formula (V), each R³² is independently selected from the group consisting of halide and unsubstituted —(C₁₋₅ alkyl).

In some embodiments of Formula (V), each R³³ is independently selected from the group consisting of H and unsubstituted —(C₁₋₅ alkyl).

In some embodiments of Formula (V), each R³⁴ is independently selected from the group consisting of —O(C₁₋₅ alkyl) and a heteroaryl optionally substituted with 1-6 R³⁵.

In some embodiments of Formula (V), each R³⁵ is a -heterocyclyl optionally substituted with one or more halides or one or more unsubstituted —(C₁₋₅ alkyl).

In some embodiments of Formula (V), each X is selected from the group consisting of O and S.

In some embodiments of Formula (V), Y³ is CH or nitrogen.

In some embodiments of Formula (V), Y¹, Y², Y⁴, and Y⁵ are independently selected from the group consisting of CH and nitrogen.

In some embodiments of Formula (V), if Y¹ is nitrogen then Y², Y⁴, and Y⁵ are carbon, Y³ is CH, and R⁴ is absent.

In some embodiments of Formula (V), if Y² is nitrogen then Y¹, Y⁴, and Y⁵ are carbon, Y³ is CH, and R⁵ is absent.

In some embodiments of Formula (V), if Y³ is nitrogen then Y¹, Y², Y⁴, and Y⁵ are carbon.

In some embodiments of Formula (V), if Y⁴ is nitrogen then Y¹, Y², and Y⁵ are carbon, Y³ is CH, and R¹ is absent.

In some embodiments of Formula (V), if Y⁵ is nitrogen then Y¹, Y², and Y⁴ are carbon, Y³ is CH, and R² is absent.

In some embodiments of Formula (V), each p is independently 0 or 1.

In some embodiments of the compounds of Formula (V), or pharmaceutically acceptable salts or solvates thereof:

Y¹, Y², Y⁴, and Y⁵ are each CH;

R³ is monocyclic -heteroaryl optionally substituted with 1-2 R⁸;

R⁶ is selected from the group consisting of -phenyl optionally substituted with 1-2 R⁹ and -heteroaryl optionally substituted with 1-2 R¹⁰;

each R⁸ is independently selected from the group consisting of H, F, methyl, —CF₃, —NH₂, —(C₁₋₄ alkylene)ₚXR¹⁹, —(C₁₋₄ alkylene)ₚheterocyclyl optionally substituted with 1-2 R²⁰, and unsubstituted -carbocyclyl; wherein each —(C₁₋₄ alkylene) is unsubstituted;

each R⁹ is independently selected from the group consisting of F, unsubstituted —(C₁₋₉ alkyl), —XR²³, —C(═O)N(R¹⁵)₂, —(C₁₋₄ alkylene)ₚN(R²⁴)₂, —(C₁₋₄ alkylene)ₚheterocyclyl optionally substituted with 1-2 R²², and —(C₁₋₄ alkylene)ₚcarbocyclyl optionally substituted with 1-2 R²¹; wherein each —(C₁₋₄ alkylene) is unsubstituted;

each R¹⁰ is independently selected from the group consisting of F, unsubstituted —(C₁₋₉ alkyl), —XR²³, —C(═O)N(R¹⁵)₂, —(C₁₋₄ alkylene)ₚN(R²⁴)₂, —(C₁₋₄ alkylene)ₚheterocyclyl optionally substituted with 1-2 R²², and —(C₁₋₄ alkylene)ₚcarbocyclyl optionally substituted with 1-2 R²¹; wherein each —(C₁₋₄ alkylene) is unsubstituted;

each R¹⁵ is selected from the group consisting of H and unsubstituted —(C₁₋₅ alkyl);

each R¹⁹ is independently selected from the group consisting of H and unsubstituted —(C₁₋₅ alkyl);

each R²⁰ independently is selected from the group consisting of F, unsubstituted —(C₁₋₅ alkyl), unsubstituted —(C₁₋₅ haloalkyl), —OH, —N(R¹⁵)₂;

each R²¹ is independently selected from the group consisting of F, unsubstituted —(C₁₋₅ alkyl), and unsubstituted —(C₁₋₅ haloalkyl);

each R²² is independently selected from the group consisting of F, unsubstituted —(C₁₋₅ alkyl), unsubstituted —(C₁₋₅ haloalkyl), —OH, —N(R¹⁵)₂;

each R²³ is independently selected from the group consisting of H, unsubstituted —(C₁₋₅ alkyl), unsubstituted —(C₁₋₅ haloalkyl), —(C₁₋₄ alkylene)N(R¹⁵)₂;

each R²⁴ is independently selected from the group consisting of H, unsubstituted —(C₁₋₅ alkyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)N(R¹⁵)₂; wherein each —(C₁₋₄ alkylene) is unsubstituted;

each X is selected from the group consisting of O and S; and

each p is independently 0 or 1.

U.S. Provisional Application No. 62/685,764 describes compounds having Formula (VI) and is hereby incorporated by reference in its entirety.

Some embodiments of the present disclosure include compounds of Formula (VI):

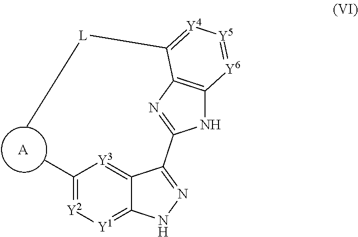

or pharmaceutically acceptable salts or solvates thereof.

In some embodiments of Formula (VI), Ring A is a 5-6-membered heteroaryl optionally substituted with 1-4 (e.g., 1-3, 1-2, 1) R¹.

In some embodiments of Formula (VI), L is -L¹-L²-L³-L⁴-.

In some embodiments of Formula (VI), L¹ is selected from the group consisting of unsubstituted —(C₁₋₃ alkylene)-, —NR²—, —NR³(C═O)—, —(C═O)NR³—, and —O—.

In some embodiments of Formula (VI), L² is selected from the group consisting of unsubstituted —(C₁₋₆ alkylene)- and —NR²—.

In some embodiments of Formula (VI), L³ is selected from the group consisting of unsubstituted —(C₁₋₆ alkylene)-, —O—, and -carbocyclylene- optionally substituted with one or more halides.

In some embodiments of Formula (VI), L⁴ is selected from the group consisting of unsubstituted —(C₁₋₆ alkylene)-, —O—, —NR²—, —NR³(C═O)—, —(C═O)NR³—, -arylene- substituted with 1-5 R⁴, and -heteroarylene- optionally substituted with 1-4 (e.g., 1-3, 1-2, 1) R⁵.

In some embodiments of Formula (VI), there is the proviso that —NR²— and —O— are not adjacent to each other.

In some embodiments of Formula (VI), there is the proviso that two —NR²— and/or two —O— are not adjacent to each other.

In some embodiments of Formula (VI), there is the proviso that two —NR³(C═O)— and/or —(C═O)NR³—, are not adjacent to each other.

In some embodiments of Formula (VI), each R¹ is selected from the group consisting of halide (e.g., F, Cl, Br, I), unsubstituted —(C₁₋₃ alkyl), unsubstituted —(C₁₋₃ haloalkyl), and —CN.

In some embodiments of Formula (VI), each R² is selected from the group consisting of H and unsubstituted —(C₁₋₆ alkyl).

In some embodiments of Formula (VI), each R³ is selected from the group consisting of H and unsubstituted —(C₁₋₆ alkyl).

In some embodiments of Formula (VI), each R⁴ is selected from the group consisting of halide (e.g., F, Cl, Br, I), unsubstituted —(C₁₋₆ alkyl), unsubstituted —(C₁₋₆ haloalkyl), and —CN.

In some embodiments of Formula (VI), each R⁵ is selected from the group consisting of halide (e.g., F, Cl, Br, I), unsubstituted —(C₁₋₆ alkyl), unsubstituted —(C₁₋₆ haloalkyl), and —CN.

In some embodiments of Formula (VI), Y¹, Y², Y³, Y⁴, Y⁵, and Y⁶ are independently selected from the group consisting of CH and nitrogen.

In some embodiments of Formula (VI), if Y¹ is nitrogen then Y² and Y³ are CH.

In some embodiments of Formula (VI), if Y² is nitrogen then Y¹ and Y³ are CH.

In some embodiments of Formula (VI), if Y³ is nitrogen then Y¹ and Y² are CH.

In some embodiments of Formula (VI), if Y⁴ is nitrogen then Y⁵ and Y⁶ are CH.

In some embodiments of Formula (VI), if Y⁵ is nitrogen then Y⁴ and Y⁶ are CH.

In some embodiments of Formula (VI), if Y⁶ is nitrogen then Y⁴ and Y⁵ are CH.

In some embodiments of the compounds of Formula (VI), or pharmaceutically acceptable salts or solvates thereof:

Y¹, Y², and Y³ are CH;

Y⁴, Y⁵, and Y⁶ are independently selected from the group consisting of CH and nitrogen;

Ring A is selected from the group consisting of pyridinyl, pyrimidinyl, pyrazinyl, and pyridazinyl, each optionally substituted with 1-2 R¹;

L is -L¹-L²-L³-L⁴-;

L¹ is selected from the group consisting of unsubstituted —(C₁₋₃ alkylene)-, —NR²—, —NR³(C═O)—, —(C═O)NR³—, and —O—;

L² is selected from the group consisting of unsubstituted —(C₁₋₆ alkylene)- and —NR²—;

L³ is selected from the group consisting of unsubstituted —(C₁₋₆ alkylene)-, —O—, and unsubstituted -carbocyclylene-;

L⁴ is selected from the group consisting of unsubstituted —(C₁₋₆ alkylene)-, —O—, —NR²—, —NR³(C═O)—, —(C═O)NR³—, unsubstituted -arylene-, and unsubstituted -heteroarylene;

with the proviso that —NR²— and —O— are not adjacent to each other;

with the proviso that two —NR²— and/or two —O— are not adjacent to each other;

with the proviso that two —NR³(C═O)— and/or —(C═O)NR³—, are not adjacent to each other;

each R¹ is selected from the group consisting of F, unsubstituted —(C₁₋₃ alkyl), unsubstituted —(C₁₋₃haloalkyl), and —CN;

each R² is selected from the group consisting of H and methyl; and

each R³ is selected from the group consisting of H and methyl.

U.S. Provisional Application No. 62/685,764 describes compounds having Formula (VII) and is hereby incorporated by reference in its entirety.

Some embodiments of the present disclosure include compounds of Formula (VII):

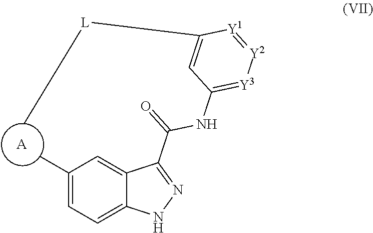

or pharmaceutically acceptable salts or solvates thereof.

In some embodiments of Formula (VII), Ring A is a 5-6-membered heteroaryl optionally substituted with 1-3 R¹.

In some embodiments of Formula (VII), L is -L¹-L²-L³-L⁴-.

In some embodiments of Formula (VII), L¹ is selected from the group consisting of unsubstituted —(C₁₋₃ alkylene)-, —NR²—, —NR³(C═O)—, —(C═O)NR³—, and —O—.

In some embodiments of Formula (VII), L² is selected from the group consisting of unsubstituted —(C₁₋₆ alkylene)-, —NR²—, —NR³(C═O)—, and —(C═O)NR³—.

In some embodiments of Formula (VII), L³ is selected from the group consisting of unsubstituted —(C₁₋₆ alkylene)-, —O—, and carbocyclylene optionally substituted with one or more halides.

In some embodiments of Formula (VII), L⁴ is selected from the group consisting of unsubstituted —(C₁₋₆ alkylene)-, —O—, —NR²—, —NR³(C═O)—, —(C═O)NR³—, -arylene substituted with 1-5 (e.g., 1-4, 1-3, 1-2, 1) R⁴, and -heteroarylene optionally substituted with 1-4 (e.g., 1-3, 1-2, 1) R⁵.

In some embodiments of Formula (VII), there is the proviso that —NR²— and —O— are not adjacent to each other.

In some embodiments of Formula (VII), there is the proviso that two —NR²— and/or two —O— are not adjacent to each other.

In some embodiments of Formula (VII), there is the proviso that two —NR³(C═O)— and/or —(C═O)NR³—, are not adjacent to each other.

In some embodiments of Formula (VII), each R¹ is selected from the group consisting of halide (e.g., F, Cl, Br, I), unsubstituted —(C₁₋₃ alkyl), unsubstituted —(C₁₋₃ haloalkyl), and —CN.

In some embodiments of Formula (VII), each R² is selected from the group consisting of H and unsubstituted —(C₁₋₆ alkyl).

In some embodiments of Formula (VII), each R³ is selected from the group consisting of H and unsubstituted —(C₁₋₆ alkyl).

In some embodiments of Formula (VII), each R⁴ is selected from the group consisting of halide (e.g., F, Cl, Br, I), unsubstituted —(C₁₋₆ alkyl), unsubstituted —(C₁₋₆ haloalkyl), and —CN.

In some embodiments of Formula (VII), each R⁵ is selected from the group consisting of halide (e.g., F, Cl, Br, I), unsubstituted —(C₁₋₆ alkyl), unsubstituted —(C₁₋₆ haloalkyl), and —CN.

In some embodiments of Formula (VII), Y¹, Y², and Y³ are independently selected from the group consisting of CH and nitrogen.

In some embodiments of Formula (VII), if Y¹ is nitrogen then Y² and Y³ are CH.

In some embodiments of Formula (VII), if Y² is nitrogen then Y¹ and Y³ are CH.

In some embodiments of Formula (VII), if Y³ is nitrogen then Y¹ and Y² are CH.

In some embodiments of the compounds of Formula (VII), or pharmaceutically acceptable salts or solvates thereof:

Ring A is pyridine optionally substituted with 1-2 R¹;

L is -L¹-L²-L³-L⁴-;

L¹ is selected from the group consisting of unsubstituted —(C₁₋₃ alkylene)-, —NR²— —NR³(C═O)—, —(C═O)NR³—, and —O—;

L² is selected from the group consisting of unsubstituted —(C₁₋₆ alkylene)-, —NR²—, —NR³(C═O)—, and —(C═O)NR³—;

L³ is selected from the group consisting of unsubstituted —(C₁₋₆ alkylene)-, and —O—;

L⁴ is selected from the group consisting of unsubstituted -arylene substituted and unsubstituted -heteroarylene;

with the proviso that —NR²— and —O— are not adjacent to each other;

with the proviso that two —NR²— and/or two —O— are not adjacent to each other;

with the proviso that two —NR³(C═O)— and/or —(C═O)NR³—, are not adjacent to each other;

each R¹ is selected from the group consisting of F, unsubstituted —(C₁₋₃ alkyl), unsubstituted —(C₁₋₃haloalkyl), and —CN;

each R² is selected from the group consisting of H and methyl;

each R³ is selected from the group consisting of H and methyl; and

Y¹, Y², and Y³ are independently selected from the group consisting of CH and nitrogen; wherein if Y¹ is nitrogen then Y² and Y³ are CH.

EMBO Molecular Medicine (2018), 10(6), e8289, Journal of Medicinal Chemistry (2017), 60(21), 8989-9002, and U.S. Pat. Nos. 9,346,812 and 9,428,509 describe compounds having Formula (VIII) and are each hereby incorporated by reference in their entirety.

Some embodiments of the present disclosure include compounds of Formula (VIII):

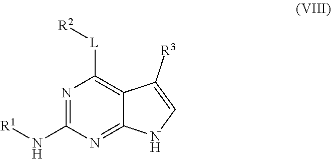

or pharmaceutically acceptable salts or solvates thereof.

In some embodiments of Formula (VIII), R¹ is selected from the group consisting of H, unsubstituted —(C₁₋₆ alkyl), unsubstituted —(C₂₋₆ alkenyl), unsubstituted —(C₂₋₆ alkynyl), and -heteroaryl optionally substituted with 1-4 (e.g., 1-3, 1-2, 1) R⁴, -aryl optionally substituted with 1-5 (e.g., 1-4, 1-3, 1-2, 1) R⁵.

In some embodiments of Formula (VIII), R² is selected from the group consisting of H, —(C₁₋₄ alkylene)ₚheteroaryl optionally substituted with 1-4 (e.g., 1-3, 1-2, 1) R⁶, —(C₁₋₄ alkylene)ₚheterocyclyl optionally substituted with 1-10 (e.g., 1-9, 1-8, 1-7, 1-6, 1-5, 1-4, 1-3, 1-2, 1) R⁷, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R⁸; wherein each —(C₁₋₄ alkylene) is, independently, optionally substituted with one or more substituents as defined anywhere herein.

In some embodiments of Formula (VIII), R³ is selected from the group consisting of -heteroaryl optionally substituted with 1-4 (e.g., 1-3, 1-2, 1) R⁹ and -aryl optionally substituted with 1-5 (e.g., 1-4, 1-3, 1-2, 1) R¹.

In some embodiments of Formula (VIII), each R⁴ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R¹³, —SO₂R¹⁴, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R¹⁵; wherein each —(C₁₋₄ alkylene) is, independently, optionally substituted with one or more substituents as defined anywhere herein.

In some embodiments of Formula (VIII), each R⁵ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆haloalkyl), —OR¹¹, —C(═O)N(R¹²)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R¹³, —SO₂R¹⁴, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R¹⁵; wherein each —(C₁₋₄ alkylene) is, independently, optionally substituted with one or more substituents as defined anywhere herein.

In some embodiments of Formula (VIII), each R⁶ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴.

In some embodiments of Formula (VIII), each R⁷ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (VIII), each R⁸ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (VIII), each R⁹ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴.

In some embodiments of Formula (VIII), each R¹⁰ is independently selected from the group consisting of halide (e.g., F, Cl, Br, I), —CN, unsubstituted —(C₁₋₆ alkyl), unsubstituted —(C₂₋₆ alkenyl), unsubstituted —(C₂₋₆ alkynyl), unsubstituted —(C₁₋₆haloalkyl), —OR¹¹, —C(═O)N(R¹²)₂, and —SO₂R¹⁴.

In some embodiments of Formula (VIII), each R¹¹ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (VIII), each R¹² is independently selected from the group consisting of halide (e.g., F, Cl, Br, I), unsubstituted —(C₁₋₆ alkyl), unsubstituted —(C₂₋₆ alkenyl), and unsubstituted —(C₂₋₆ alkynyl).

In some embodiments of Formula (VIII), each R¹³ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (VIII), each R¹⁴ is independently selected from the group consisting of halide (e.g., F, Cl, Br, I), unsubstituted —(C₁₋₆ alkyl), unsubstituted —(C₂₋₆ alkenyl), and unsubstituted —(C₂₋₆ alkynyl).

In some embodiments of Formula (VIII), each R¹⁵ is independently selected from the group consisting of halide (e.g., F, Cl, Br, I), unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula (VIII), L is selected from the group consisting of a bond, —O—, and —NH—.

In some embodiments of Formula (VIII), each p is independently 0 or 1.

In some embodiments of the compounds of Formula (VIII), or pharmaceutically acceptable salts or solvates thereof:

R¹ is selected from the group consisting of H, methyl, monocyclic -heteroaryl optionally substituted with 1 R⁴, and -phenyl optionally substituted with 1 R⁵;

L is a bond or —NH—;

R² is H or —(C₁₋₂ alkylene)heteroaryl optionally substituted with 1-4 R⁵;

R³ is bicyclic -heteroaryl optionally substituted with 1-2 R⁹;

each R⁴ is independently F or —(C₁₋₄ alkylene)ₚheterocyclyl optionally substituted with 1-2 R¹³; wherein each —(C₁₋₄ alkylene) is unsubstituted;

each R⁵ is independently F or —(C₁₋₄ alkylene)ₚheterocyclyl optionally substituted with 1-2 R¹³; wherein each —(C₁₋₄ alkylene) is unsubstituted;

R⁶ is independently selected from the group consisting of F, —CN, methyl, —CF₃, and —OR¹¹.

each R⁹ is independently selected from the group consisting of F, —CN, unsubstituted —(C₁₋₆ alkyl), unsubstituted —(C₁₋₆ haloalkyl), and —OR¹¹;

each R¹¹ is independently selected from the group consisting of unsubstituted —(C₁₋₆ alkyl) and unsubstituted —(C₁₋₆haloalkyl);

each R¹³ is independently selected from the group consisting of F, unsubstituted —(C₁₋₆ alkyl), and unsubstituted —(C₁₋₆ haloalkyl); and

each p is independently 0 or 1.

In some embodiments of Formulas (I)-(VIII), each p is 0 or 1; in some embodiments of Formulas (I)-(VIII), p is 0; in some embodiments of Formulas (I)-(VIII), p is 1.

In some embodiments of Formulas (I)-(VIII), each —(C₁₋₄ alkylene) is —(C₁₋₃ alkylene).

In some embodiments of Formulas (I)-(VIII), each —(C₁₋₄ alkylene) is —(C₁₋₂ alkylene).

In some embodiments of Formulas (I)-(VIII), each —(C₁₋₄ alkylene) is —(C₁ alkylene).

In some embodiments of Formulas (I)-(VIII), each —(C₁₋₄ alkylene) is —CH₂—.

In some embodiments of Formulas (I)-(VIII), each —(C₁₋₄ alkylene) is optionally substituted with halide (e.g., F, Cl, Br, I).

In some embodiments of Formulas (I)-(VIII), each —(C₁₋₄ alkylene) is optionally substituted with F.

Illustrative compounds of Formulas (I)-(VIII) are shown in Table 3.

TABLE 3

| | |
|---|---|
| 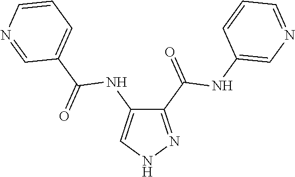 [SMILES: O=C(Nc1c[nH]nc1C(=O)Nc1cccnc1)c1cccnc1] | 1 |
| 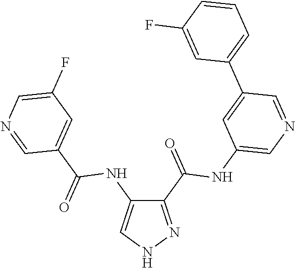 [SMILES: O=C(Nc1c[nH]nc1C(=O)Nc1cncc(-c2cccc(F)c2)c1)c1cncc(F)c1] | 2 |
| 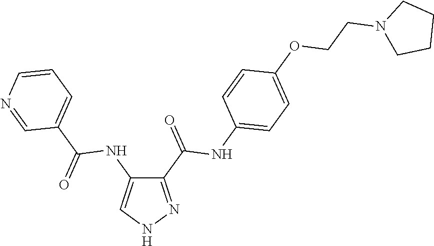 [SMILES: O=C(Nc1c[nH]nc1C(=O)Nc1ccc(OCCN2CCCC2)cc1)c1cccnc1] | 3 |
| 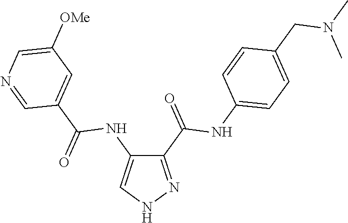 [SMILES: COc1cncc(C(=O)Nc2c[nH]nc2C(=O)Nc2ccc(CN(C)C)cc2)c1] | 4 |
| 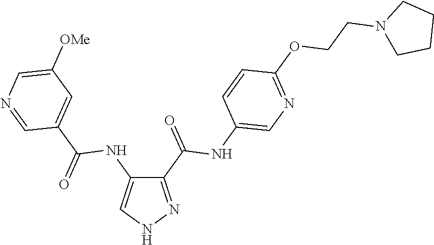 [SMILES: COc1cncc(C(=O)Nc2c[nH]nc2C(=O)Nc2ccc(OCCN3CCCC3)nc2)c1] | 5 |
| 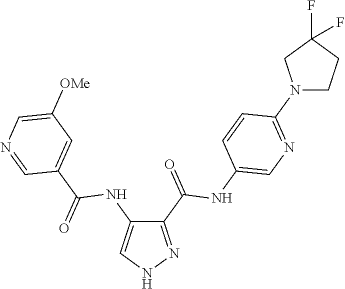 [SMILES: COc1cncc(C(=O)Nc2c[nH]nc2C(=O)Nc2ccc(N3CCC(F)(F)C3)nc2)c1] | 6 |
| 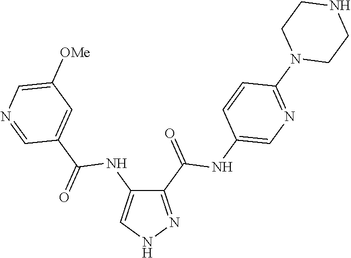 [SMILES: COc1cncc(C(=O)Nc2c[nH]nc2C(=O)Nc2ccc(N3CCNCC3)nc2)c1] | 7 |
| 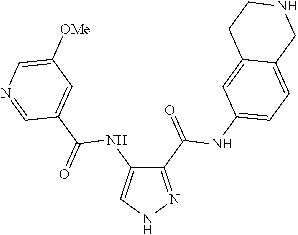 [SMILES: COc1cncc(C(=O)Nc2c[nH]nc2C(=O)Nc2ccc3c(c2)CCNC3)c1] | 8 |
| 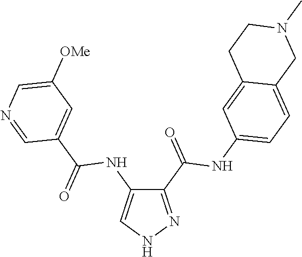 [SMILES: COc1cncc(C(=O)Nc2c[nH]nc2C(=O)Nc2ccc3c(c2)CCN(C)C3)c1] | 9 |
| 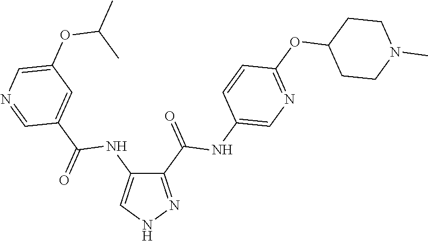 [SMILES: CC(C)Oc1cncc(C(=O)Nc2c[nH]nc2C(=O)Nc2ccc(OC3CCN(C)CC3)nc2)c1] | 10 |
| 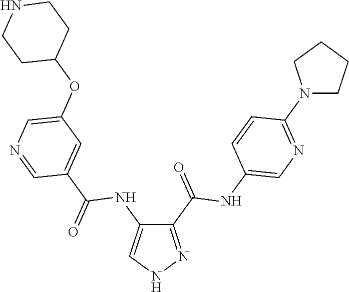 [SMILES: O=C(Nc1c[nH]nc1C(=O)Nc1ccc(N2CCCC2)nc1)c1cncc(OC2CCNCC2)c1] | 11 |
| 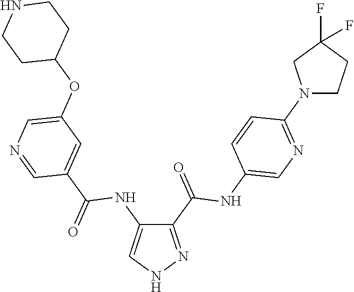 [SMILES: O=C(Nc1c[nH]nc1C(=O)Nc1ccc(N2CCC(F)(F)C2)nc1)c1cncc(OC2CCNCC2)c1] | 12 |
| 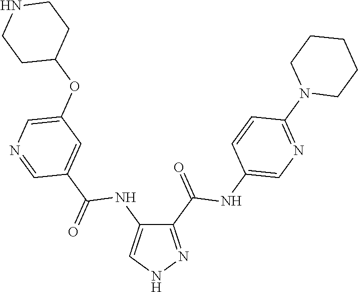 [SMILES: O=C(Nc1c[nH]nc1C(=O)Nc1ccc(N2CCCCC2)nc1)c1cncc(OC2CCNCC2)c1] | 13 |
| 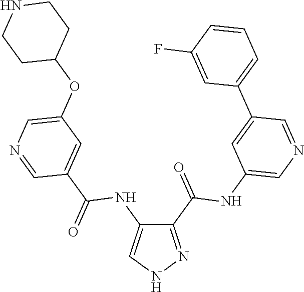 [SMILES: O=C(Nc1c[nH]nc1C(=O)Nc1cncc(-c2cccc(F)c2)c1)c1cncc(OC2CCNCC2)c1] | 14 |
| 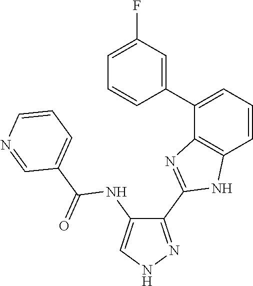 [SMILES: O=C(Nc1c[nH]nc1-c1nc2c(-c3cccc(F)c3)cccc2[nH]1)c1cccnc1] | 15 |
| 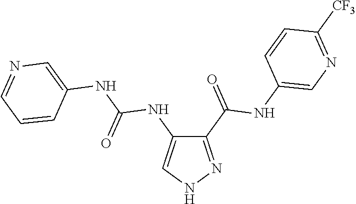 [SMILES: O=C(Nc1cccnc1)Nc1c[nH]nc1C(=O)Nc1ccc(C(F)(F)F)nc1] | 16 |
| 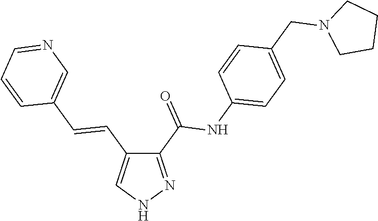 [SMILES: O=C(Nc1ccc(CN2CCCC2)cc1)c1n[nH]cc1/C=C/c1cccnc1] | 17 |
| 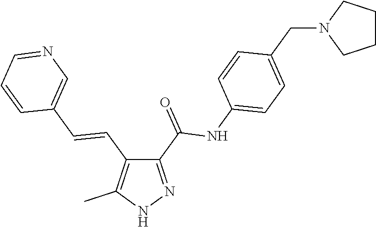 [SMILES: Cc1[nH]nc(C(=O)Nc2ccc(CN3CCCC3)cc2)c1/C=C/c1cccnc1] | 18 |
| 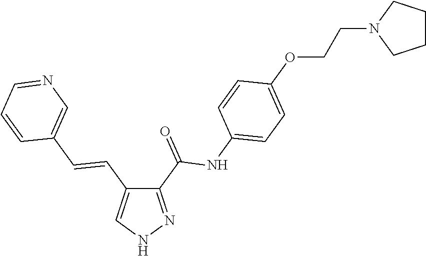 [SMILES: O=C(Nc1ccc(OCCN2CCCC2)cc1)c1n[nH]cc1/C=C/c1cccnc1] | 19 |
| 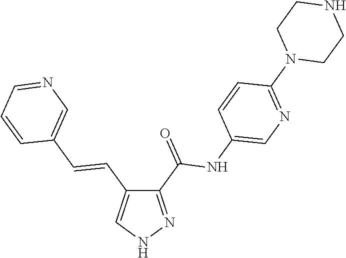 [SMILES: O=C(Nc1ccc(N2CCNCC2)nc1)c1n[nH]cc1/C=C/c1cccnc1] | 20 |
| 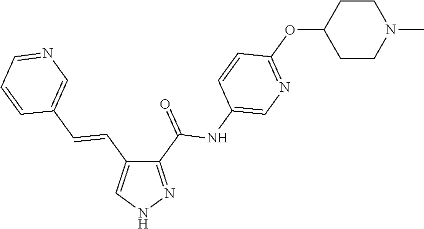 [SMILES: CN1CCC(Oc2ccc(NC(=O)c3n[nH]cc3/C=C/c3cccnc3)cn2)CC1] | 21 |
| 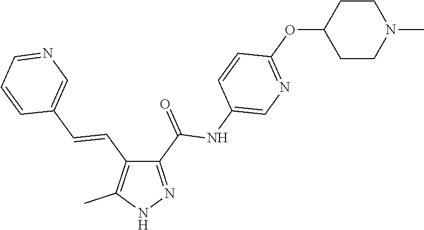 [SMILES: Cc1[nH]nc(C(=O)Nc2ccc(OC3CCN(C)CC3)nc2)c1/C=C/c1cccnc1] | 22 |
| 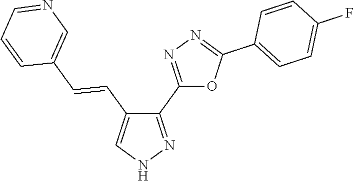 [SMILES: Fc1ccc(-c2nnc(-c3n[nH]cc3/C=C/c3cccnc3)o2)cc1] | 23 |
| 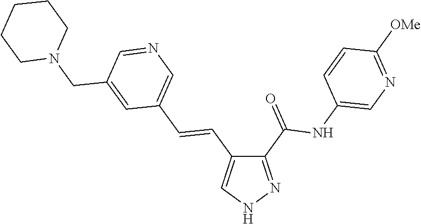 [SMILES: COc1ccc(NC(=O)c2n[nH]cc2/C=C/c2cncc(CN3CCCCC3)c2)cn1] | 24 |
| 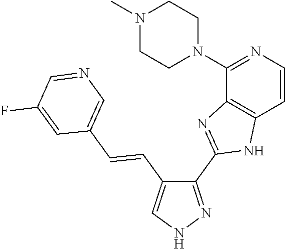 [SMILES: CN1CCN(c2nccc3[nH]c(-c4n[nH]cc4/C=C/c4cncc(F)c4)nc23)CC1] | 25 |
| 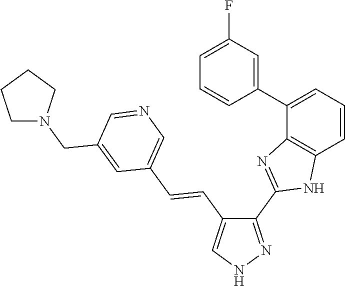 [SMILES: Fc1cccc(-c2cccc3[nH]c(-c4n[nH]cc4/C=C/c4cncc(CN5CCCC5)c4)nc23)c1] | 26 |
| 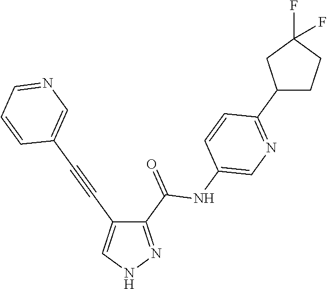 [SMILES: O=C(Nc1ccc(C2CCC(F)(F)C2)nc1)c1n[nH]cc1C#Cc1cccnc1] | 27 |
| 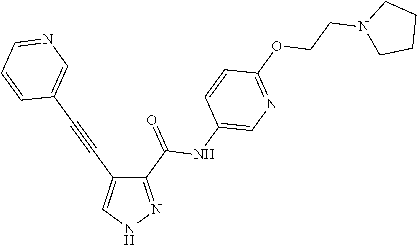 [SMILES: O=C(Nc1ccc(OCCN2CCCC2)nc1)c1n[nH]cc1C#Cc1cccnc1] | 28 |
| 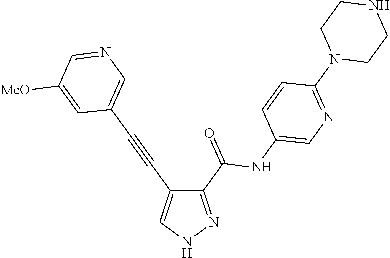 [SMILES: COc1cncc(C#Cc2c[nH]nc2C(=O)Nc2ccc(N3CCNCC3)nc2)c1] | 29 |
| 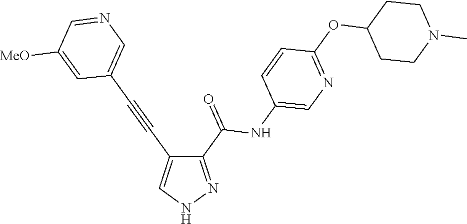 [SMILES: COc1cncc(C#Cc2c[nH]nc2C(=O)Nc2ccc(OC3CCN(C)CC3)nc2)c1] | 30 |
| 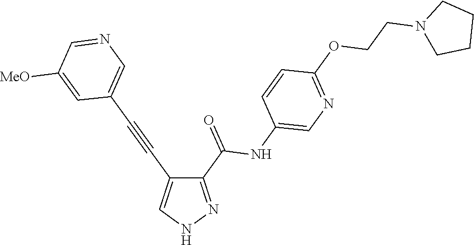 [SMILES: COc1cncc(C#Cc2c[nH]nc2C(=O)Nc2ccc(OCCN3CCCC3)nc2)c1] | 31 |
| 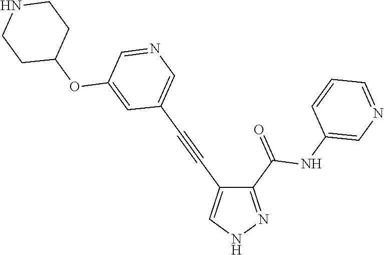 [SMILES: O=C(Nc1cccnc1)c1n[nH]cc1C#Cc1cncc(OC2CCNCC2)c1] | 32 |
| 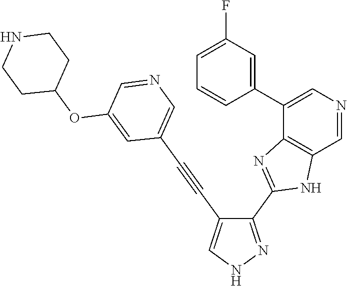 [SMILES: Fc1cccc(-c2cncc3[nH]c(-c4n[nH]cc4C#Cc4cncc(OC5CCNCC5)c4)nc23)c1] | 33 |
| 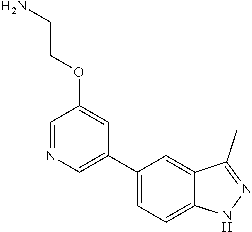 [SMILES: Cc1n[nH]c2ccc(-c3cncc(OCCN)c3)cc12] | 34 |
| 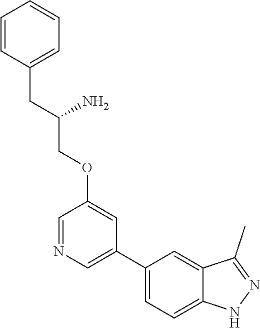 [SMILES: Cc1n[nH]c2ccc(-c3cncc(OC[C@@H](N)Cc4ccccc4)c3)cc12] | 35 |
| 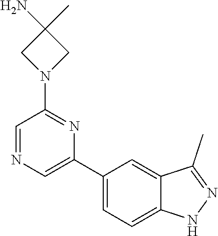 [SMILES: Cc1n[nH]c2ccc(-c3cncc(N4CC(C)(N)C4)n3)cc12] | 36 |
| 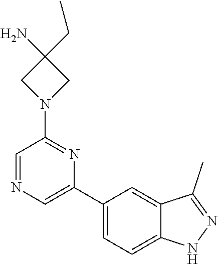 [SMILES: CCC1(N)CN(c2cncc(-c3ccc4[nH]nc(C)c4c3)n2)C1] | 37 |
| 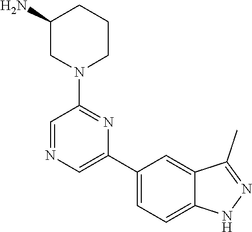 [SMILES: Cc1n[nH]c2ccc(-c3cncc(N4CCC[C@H](N)C4)n3)cc12] | 38 |
| 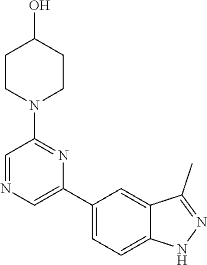 [SMILES: Cc1n[nH]c2ccc(-c3cncc(N4CCC(O)CC4)n3)cc12] | 39 |
| 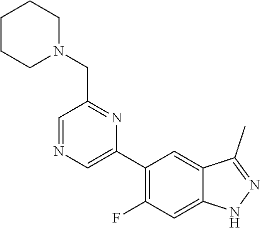 [SMILES: Cc1n[nH]c2cc(F)c(-c3cncc(CN4CCCCC4)n3)cc12] | 40 |
| 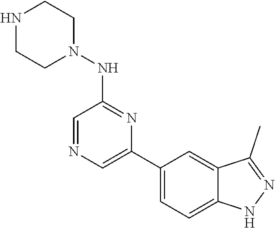 [SMILES: Cc1n[nH]c2ccc(-c3cncc(NN4CCNCC4)n3)cc12] | 41 |
| 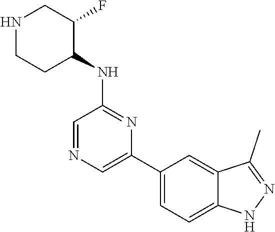 | 42 |
| 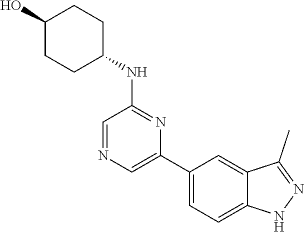 [SMILES: Cc1n[nH]c2ccc(-c3cncc(N[C@H]4CC[C@H](O)CC4)n3)cc12] | 43 |
| 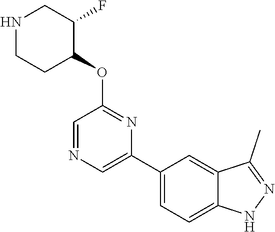 | 44 |
| 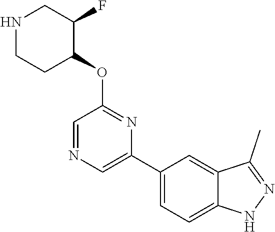 | 45 |
| 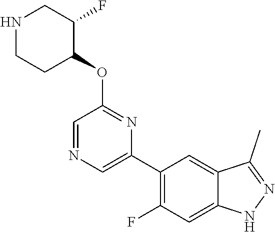 | 46 |
| 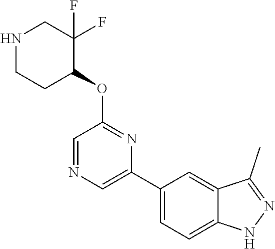 | 47 |
| 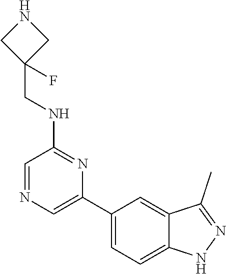 [SMILES: Cc1n[nH]c2ccc(-c3cncc(NCC4(F)CNC4)n3)cc12] | 48 |
| 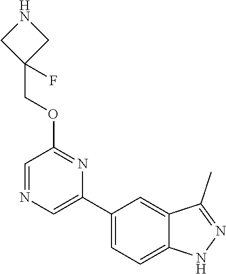 [SMILES: Cc1n[nH]c2ccc(-c3cncc(OCC4(F)CNC4)n3)cc12] | 49 |
| 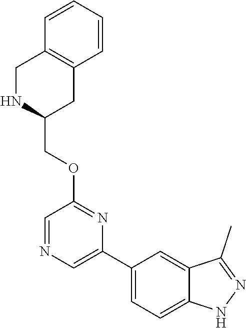 [SMILES: Cc1n[nH]c2ccc(-c3cncc(OC[C@@H]4Cc5ccccc5CN4)n3)cc12] | 50 |
| 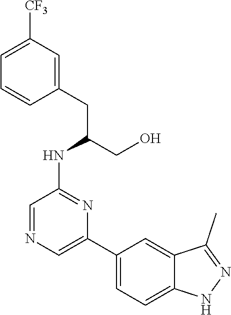 | 51 |
| 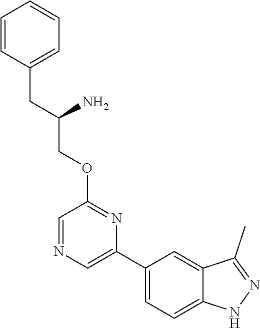 [SMILES: Cc1n[nH]c2ccc(-c3cncc(OC[C@H](N)Cc4ccccc4)n3)cc12] | 52 |
| 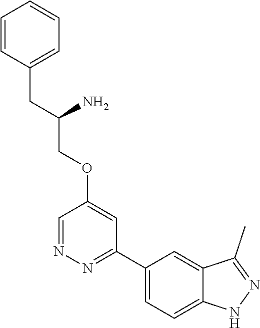 [SMILES: Cc1n[nH]c2ccc(-c3cc(OC[C@H](N)Cc4ccccc4)cnn3)cc12] | 53 |
| 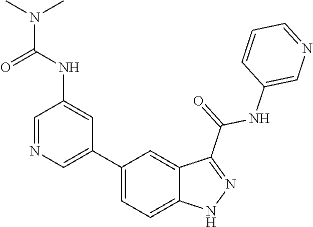 [SMILES: CN(C)C(=O)Nc1cncc(-c2ccc3[nH]nc(C(=O)Nc4cccnc4)c3c2)c1] | 54 |
| 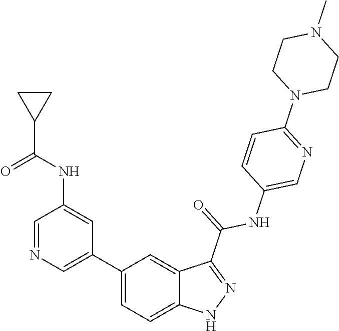 [SMILES: CN1CCN(c2ccc(NC(=O)c3n[nH]c4ccc(-c5cncc(NC(=O)C6CC6)c5)cc34)cn2)CC1] | 55 |
| 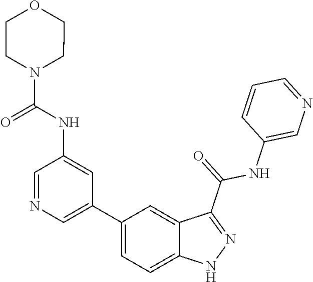 [SMILES: O=C(Nc1cccnc1)c1n[nH]c2ccc(-c3cncc(NC(=O)N4CCOCC4)c3)cc12] | 56 |
| 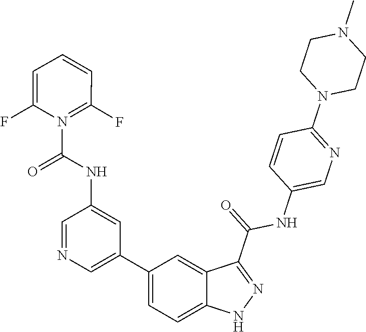 | 57 |
| 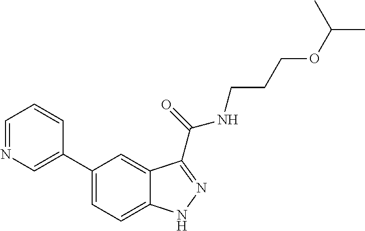 [SMILES: CC(C)OCCCNC(=O)c1n[nH]c2ccc(-c3cccnc3)cc12] | 58 |
| 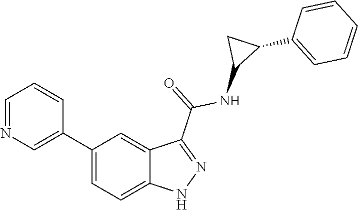 | 59 |
| 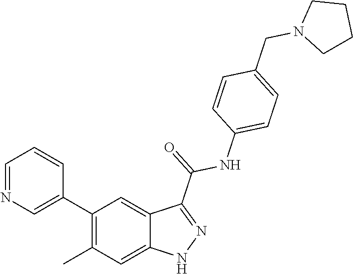 [SMILES: Cc1cc2[nH]nc(C(=O)Nc3ccc(CN4CCCC4)cc3)c2cc1-c1cccnc1] | 60 |
| 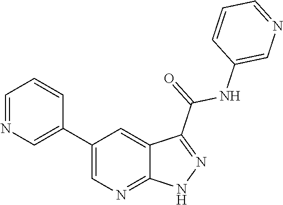 [SMILES: O=C(Nc1cccnc1)c1n[nH]c2ncc(-c3cccnc3)cc12] | 61 |
| 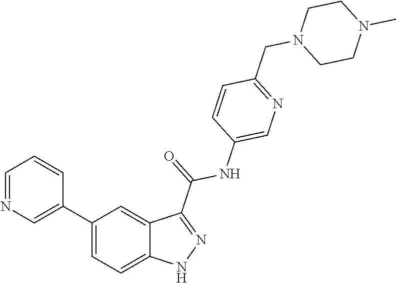 [SMILES: CN1CCN(Cc2ccc(NC(=O)c3n[nH]c4ccc(-c5cccnc5)cc34)cn2)CC1] | 62 |
| 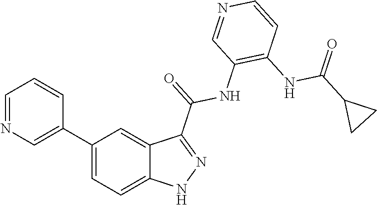 [SMILES: O=C(Nc1cnccc1NC(=O)C1CC1)c1n[nH]c2ccc(-c3cccnc3)cc12] | 63 |
| 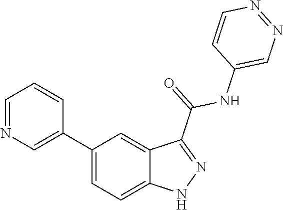 [SMILES: O=C(Nc1ccnnc1)c1n[nH]c2ccc(-c3cccnc3)cc12] | 64 |
| 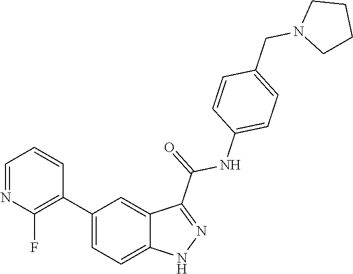 [SMILES: O=C(Nc1ccc(CN2CCCC2)cc1)c1n[nH]c2ccc(-c3cccnc3F)cc12] | 65 |
| 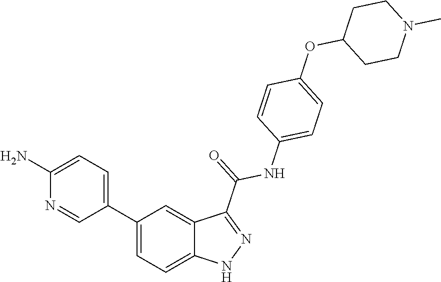 [SMILES: CN1CCC(Oc2ccc(NC(=O)c3n[nH]c4ccc(-c5ccc(N)nc5)cc34)cc2)CC1] | 66 |
| 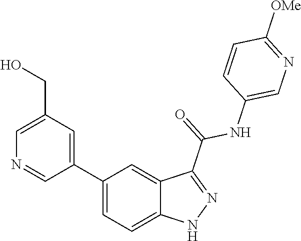 [SMILES: COc1ccc(NC(=O)c2n[nH]c3ccc(-c4cncc(CO)c4)cc23)cn1] | 67 |
| 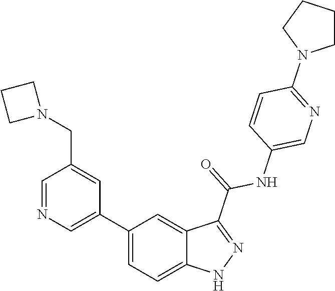 [SMILES: O=C(Nc1ccc(N2CCCC2)nc1)c1n[nH]c2ccc(-c3cncc(CN4CCC4)c3)cc12] | 68 |
| 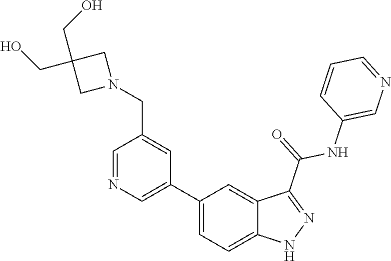 [SMILES: O=C(Nc1cccnc1)c1n[nH]c2ccc(-c3cncc(CN4CC(CO)(CO)C4)c3)cc12] | 69 |
| 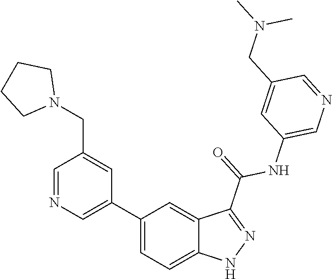 [SMILES: CN(C)Cc1cncc(NC(=O)c2n[nH]c3ccc(-c4cncc(CN5CCCC5)c4)cc23)c1] | 70 |
| 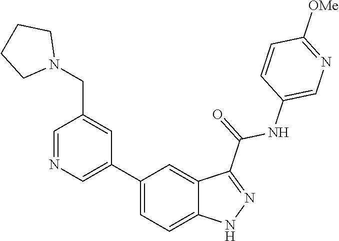 [SMILES: COc1ccc(NC(=O)c2n[nH]c3ccc(-c4cncc(CN5CCCC5)c4)cc23)cn1] | 71 |
| 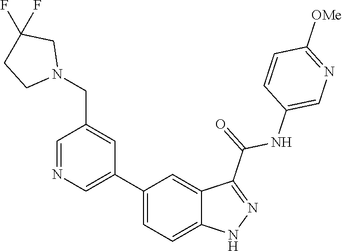 [SMILES: COc1ccc(NC(=O)c2n[nH]c3ccc(-c4cncc(CN5CCC(F)(F)C5)c4)cc23)cn1] | 72 |
| 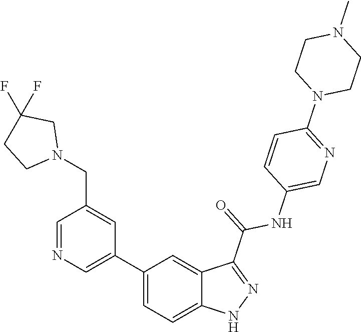 [SMILES: CN1CCN(c2ccc(NC(=O)c3n[nH]c4ccc(-c5cncc(CN6CCC(F)(F)C6)c5)cc34)cn2)CC1] | 73 |
| 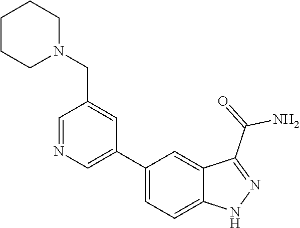 [SMILES: NC(=O)c1n[nH]c2ccc(-c3cncc(CN4CCCCC4)c3)cc12] | 74 |
| 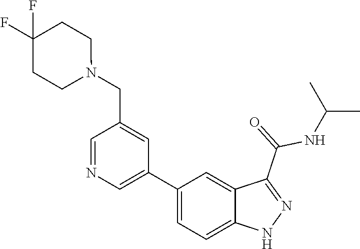 [SMILES: CC(C)NC(=O)c1n[nH]c2ccc(-c3cncc(CN4CCC(F)(F)CC4)c3)cc12] | 75 |
| 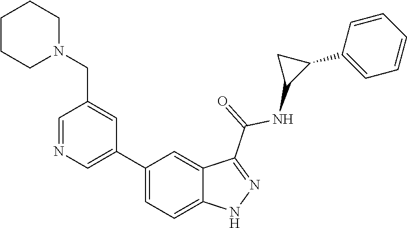 | 76 |
| 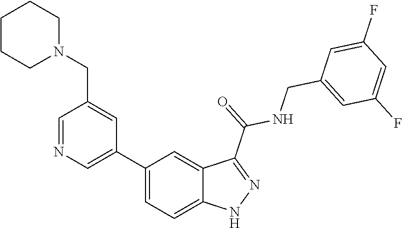 [SMILES: O=C(NCc1cc(F)cc(F)c1)c1n[nH]c2ccc(-c3cncc(CN4CCCCC4)c3)cc12] | 77 |
| 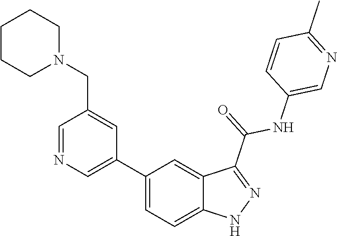 [SMILES: Cc1ccc(NC(=O)c2n[nH]c3ccc(-c4cncc(CN5CCCCC5)c4)cc23)cn1] | 78 |
| 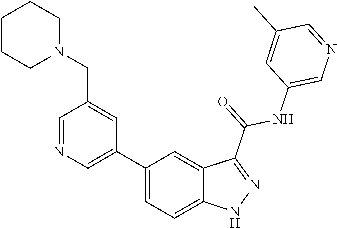 [SMILES: Cc1cncc(NC(=O)c2n[nH]c3ccc(-c4cncc(CN5CCCCC5)c4)cc23)c1] | 79 |
| 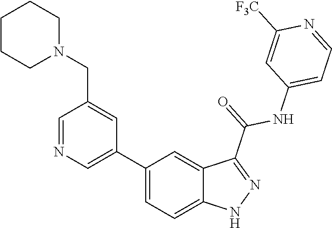 [SMILES: O=C(Nc1ccnc(C(F)(F)F)c1)c1n[nH]c2ccc(-c3cncc(CN4CCCCC4)c3)cc12] | 80 |
| 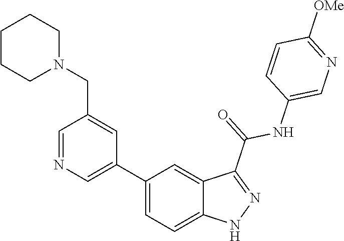 [SMILES: COc1ccc(NC(=O)c2n[nH]c3ccc(-c4cncc(CN5CCCCC5)c4)cc23)cn1] | 81 |
| 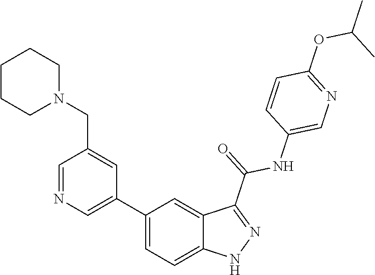 [SMILES: CC(C)Oc1ccc(NC(=O)c2n[nH]c3ccc(-c4cncc(CN5CCCCC5)c4)cc23)cn1] | 82 |
| 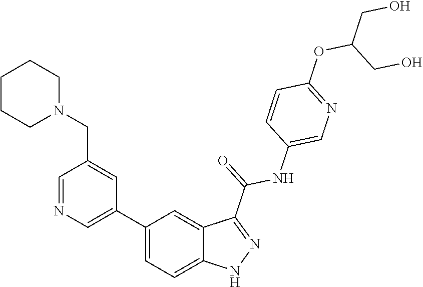 [SMILES: O=C(Nc1ccc(OC(CO)CO)nc1)c1n[nH]c2ccc(-c3cncc(CN4CCCCC4)c3)cc12] | 83 |
| 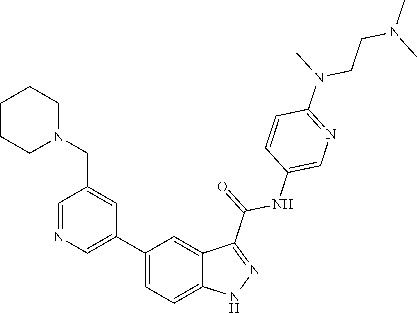 [SMILES: CN(C)CCN(C)c1ccc(NC(=O)c2n[nH]c3ccc(-c4cncc(CN5CCCCC5)c4)cc23)cn1] | 84 |
| 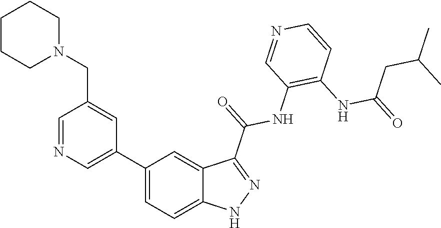 [SMILES: CC(C)CC(=O)Nc1ccncc1NC(=O)c1n[nH]c2ccc(-c3cncc(CN4CCCCC4)c3)cc12] | 85 |
| 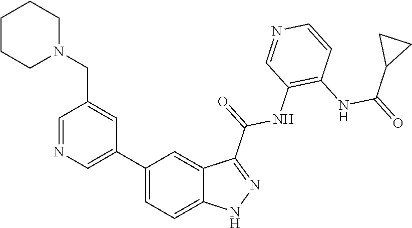 [SMILES: O=C(Nc1cnccc1NC(=O)C1CC1)c1n[nH]c2ccc(-c3cncc(CN4CCCCC4)c3)cc12] | 86 |
| 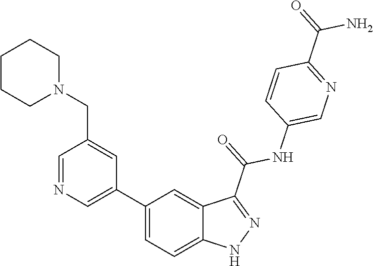 [SMILES: NC(=O)c1ccc(NC(=O)c2n[nH]c3ccc(-c4cncc(CN5CCCCC5)c4)cc23)cn1] | 87 |
| 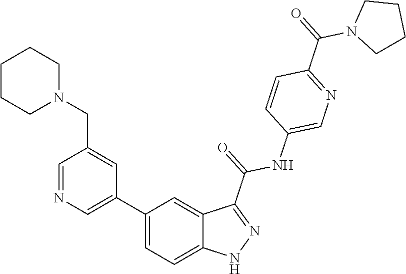 [SMILES: O=C(Nc1ccc(C(=O)N2CCCC2)nc1)c1n[nH]c2ccc(-c3cncc(CN4CCCCC4)c3)cc12] | 88 |
| 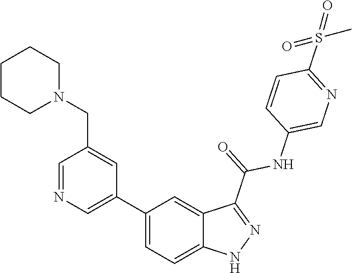 [SMILES: CS(=O)(=O)c1ccc(NC(=O)c2n[nH]c3ccc(-c4cncc(CN5CCCCC5)c4)cc23)cn1] | 89 |
| 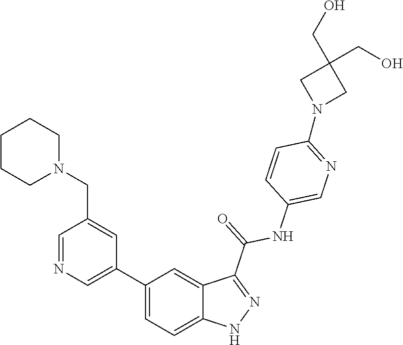 [SMILES: O=C(Nc1ccc(N2CC(CO)(CO)C2)nc1)c1n[nH]c2ccc(-c3cncc(CN4CCCCC4)c3)cc12] | 90 |
| 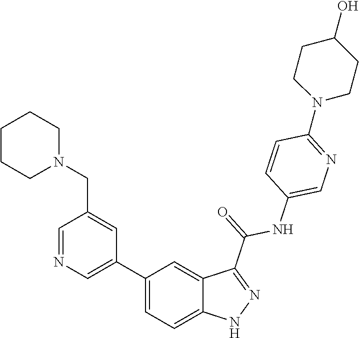 [SMILES: O=C(Nc1ccc(N2CCC(O)CC2)nc1)c1n[nH]c2ccc(-c3cncc(CN4CCCCC4)c3)cc12] | 91 |
| 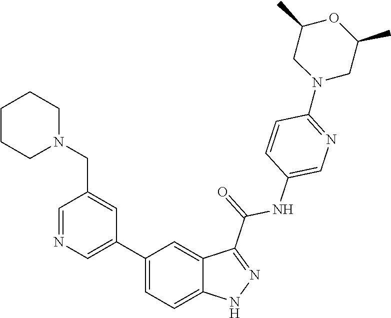 [SMILES: C[C@@H]1CN(c2ccc(NC(=O)c3n[nH]c4ccc(-c5cncc(CN6CCCCC6)c5)cc34)cn2)C[C@H](C)O1] | 92 |
| 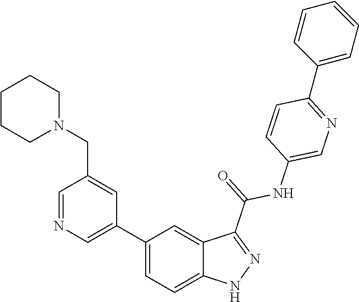 [SMILES: O=C(Nc1ccc(-c2ccccc2)nc1)c1n[nH]c2ccc(-c3cncc(CN4CCCCC4)c3)cc12] | 93 |
| 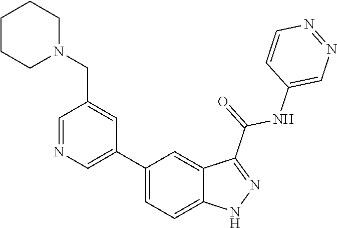 [SMILES: O=C(Nc1ccnnc1)c1n[nH]c2ccc(-c3cncc(CN4CCCCC4)c3)cc12] | 94 |
| 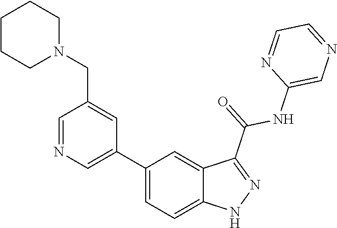 [SMILES: O=C(Nc1cnccn1)c1n[nH]c2ccc(-c3cncc(CN4CCCCC4)c3)cc12] | 95 |
| 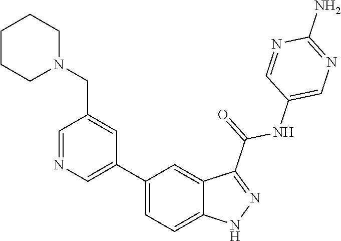 [SMILES: Nc1ncc(NC(=O)c2n[nH]c3ccc(-c4cncc(CN5CCCCC5)c4)cc23)cn1] | 96 |
| 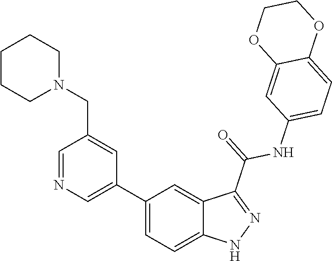 [SMILES: O=C(Nc1ccc2c(c1)OCCO2)c1n[nH]c2ccc(-c3cncc(CN4CCCCC4)c3)cc12] | 97 |
| 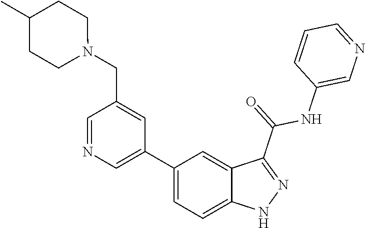 [SMILES: CC1CCN(Cc2cncc(-c3ccc4[nH]nc(C(=O)Nc5cccnc5)c4c3)c2)CC1] | 98 |
| 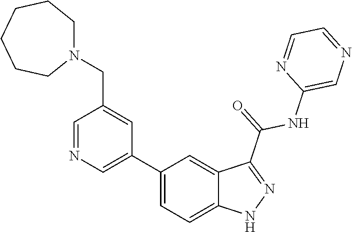 [SMILES: O=C(Nc1cnccn1)c1n[nH]c2ccc(-c3cncc(CN4CCCCCC4)c3)cc12] | 99 |
| 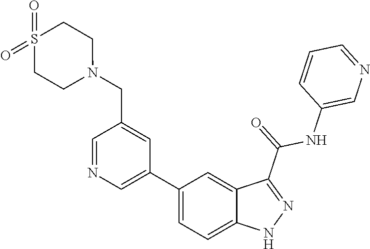 [SMILES: O=C(Nc1cccnc1)c1n[nH]c2ccc(-c3cncc(CN4CCS(=O)(=O)CC4)c3)cc12] | 100 |
| 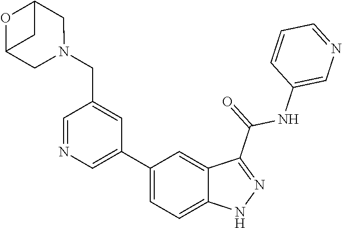 [SMILES: O=C(Nc1cccnc1)c1n[nH]c2ccc(-c3cncc(CN4CC5CC(C4)O5)c3)cc12] | 101 |
| 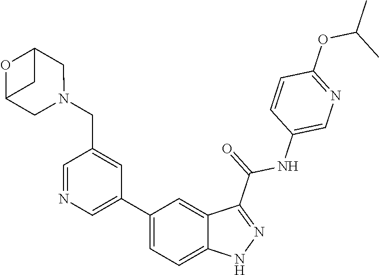 [SMILES: CC(C)Oc1ccc(NC(=O)c2n[nH]c3ccc(-c4cncc(CN5CC6CC(C5)O6)c4)cc23)cn1] | 102 |
| 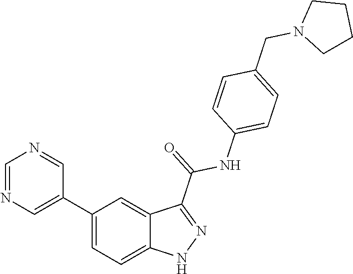 [SMILES: O=C(Nc1ccc(CN2CCCC2)cc1)c1n[nH]c2ccc(-c3cncnc3)cc12] | 103 |
| 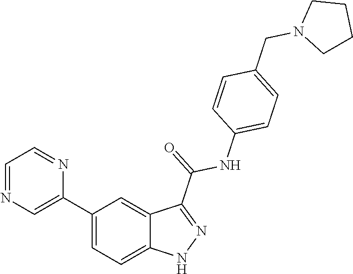 [SMILES: O=C(Nc1ccc(CN2CCCC2)cc1)c1n[nH]c2ccc(-c3cnccn3)cc12] | 104 |
| 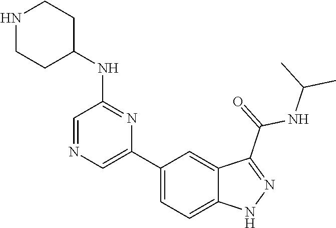 [SMILES: CC(C)NC(=O)c1n[nH]c2ccc(-c3cncc(NC4CCNCC4)n3)cc12] | 105 |
| 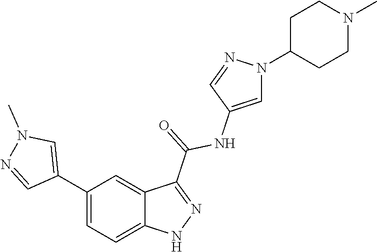 [SMILES: CN1CCC(n2cc(NC(=O)c3n[nH]c4ccc(-c5cnn(C)c5)cc34)cn2)CC1] | 106 |
| 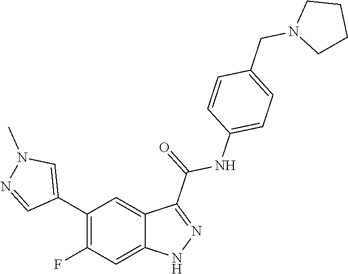 [SMILES: Cn1cc(-c2cc3c(C(=O)Nc4ccc(CN5CCCC5)cc4)n[nH]c3cc2F)cn1] | 107 |
| 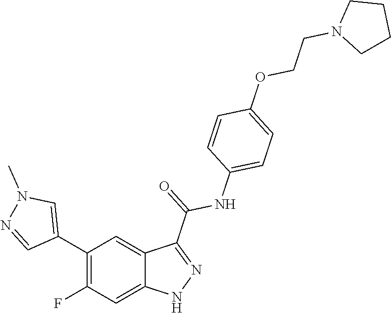 [SMILES: Cn1cc(-c2cc3c(C(=O)Nc4ccc(OCCN5CCCC5)cc4)n[nH]c3cc2F)cn1] | 108 |
| 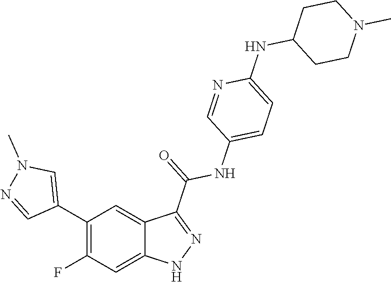 [SMILES: CN1CCC(Nc2ccc(NC(=O)c3n[nH]c4cc(F)c(-c5cnn(C)c5)cc34)cn2)CC1] | 109 |
| 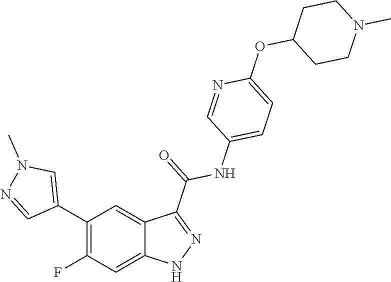 [SMILES: CN1CCC(Oc2ccc(NC(=O)c3n[nH]c4cc(F)c(-c5cnn(C)c5)cc34)cn2)CC1] | 110 |
| 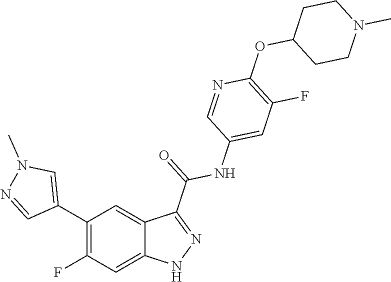 [SMILES: CN1CCC(Oc2ncc(NC(=O)c3n[nH]c4cc(F)c(-c5cnn(C)c5)cc34)cc2F)CC1] | 111 |
| 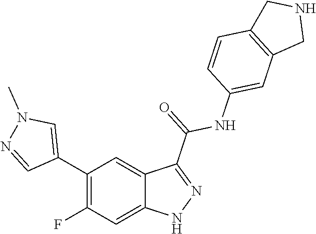 [SMILES: Cn1cc(-c2cc3c(C(=O)Nc4ccc5c(c4)CNC5)n[nH]c3cc2F)cn1] | 112 |
| 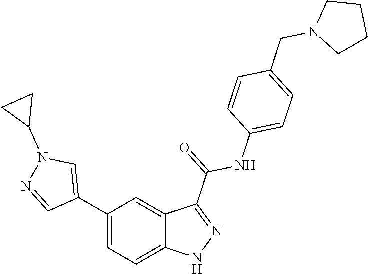 [SMILES: O=C(Nc1ccc(CN2CCCC2)cc1)c1n[nH]c2ccc(-c3cnn(C4CC4)c3)cc12] | 113 |
| 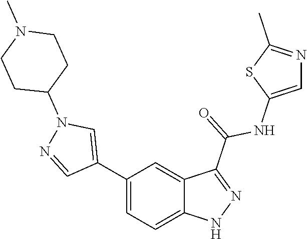 [SMILES: Cc1ncc(NC(=O)c2n[nH]c3ccc(-c4cnn(C5CCN(C)CC5)c4)cc23)s1] | 114 |
| 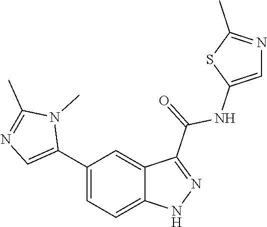 [SMILES: Cc1ncc(NC(=O)c2n[nH]c3ccc(-c4cnc(C)n4C)cc23)s1] | 115 |
| 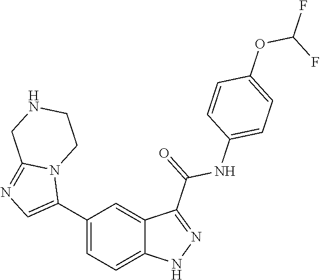 [SMILES: O=C(Nc1ccc(OC(F)F)cc1)c1n[nH]c2ccc(-c3cnc4n3CCNC4)cc12] | 116 |
| 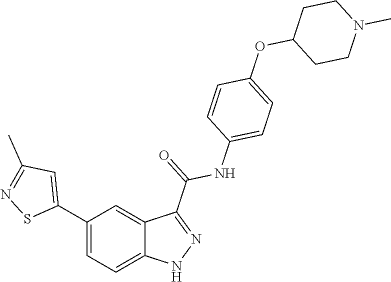 [SMILES: Cc1cc(-c2ccc3[nH]nc(C(=O)Nc4ccc(OC5CCN(C)CC5)cc4)c3c2)sn1] | 117 |
| 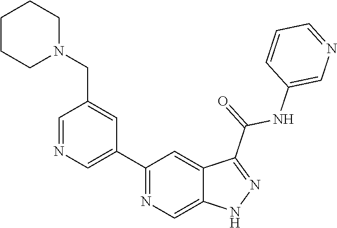 [SMILES: O=C(Nc1cccnc1)c1n[nH]c2cnc(-c3cncc(CN4CCCCC4)c3)cc12] | 118 |
| 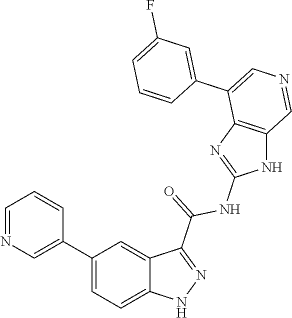 [SMILES: O=C(Nc1nc2c(-c3cccc(F)c3)cncc2[nH]1)c1n[nH]c2ccc(-c3cccnc3)cc12] | 119 |
| 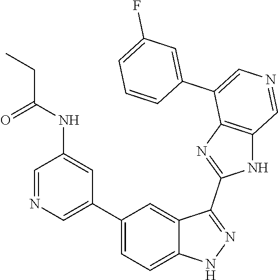 [SMILES: CCC(=O)Nc1cncc(-c2ccc3[nH]nc(-c4nc5c(-c6cccc(F)c6)cncc5[nH]4)c3c2)c1] | 120 |
| 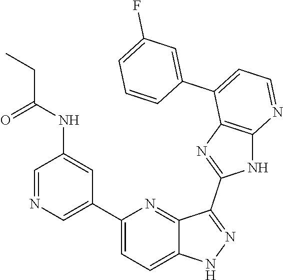 [SMILES: CCC(=O)Nc1cncc(-c2ccc3[nH]nc(-c4nc5c(-c6cccc(F)c6)ccnc5[nH]4)c3n2)c1] | 121 |
| 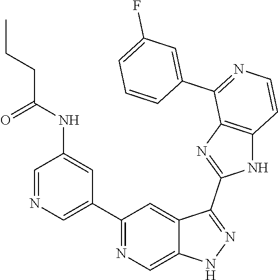 [SMILES: CCCC(=O)Nc1cncc(-c2cc3c(-c4nc5c(-c6cccc(F)c6)nccc5[nH]4)n[nH]c3cn2)c1] | 122 |
| 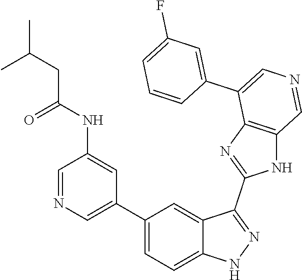 [SMILES: CC(C)CC(=O)Nc1cncc(-c2ccc3[nH]nc(-c4nc5c(-c6cccc(F)c6)cncc5[nH]4)c3c2)c1] | 123 |
| 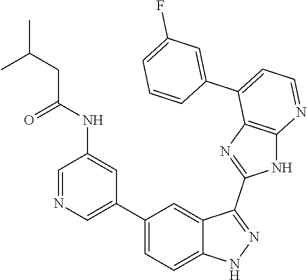 [SMILES: CC(C)CC(=O)Nc1cncc(-c2ccc3[nH]nc(-c4nc5c(-c6cccc(F)c6)ccnc5[nH]4)c3c2)c1] | 124 |
| 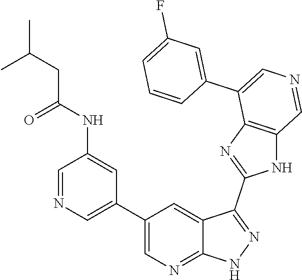 [SMILES: CC(C)CC(=O)Nc1cncc(-c2cnc3[nH]nc(-c4nc5c(-c6cccc(F)c6)cncc5[nH]4)c3c2)c1] | 125 |
| 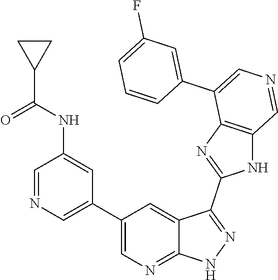 [SMILES: O=C(Nc1cncc(-c2cnc3[nH]nc(-c4nc5c(-c6cccc(F)c6)cncc5[nH]4)c3c2)c1)C1CC1] | 126 |
| 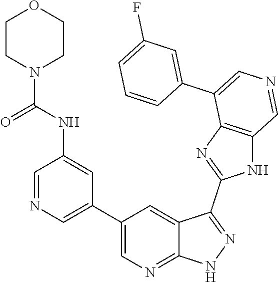 [SMILES: O=C(Nc1cncc(-c2cnc3[nH]nc(-c4nc5c(-c6cccc(F)c6)cncc5[nH]4)c3c2)c1)N1CCOCC1] | 127 |
| 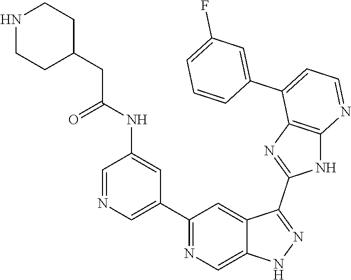 [SMILES: O=C(CC1CCNCC1)Nc1cncc(-c2cc3c(-c4nc5c(-c6cccc(F)c6)ccnc5[nH]4)n[nH]c3cn2)c1] | 128 |
| 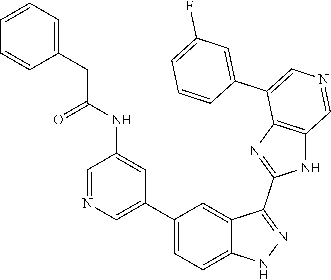 [SMILES: O=C(Cc1ccccc1)Nc1cncc(-c2ccc3[nH]nc(-c4nc5c(-c6cccc(F)c6)cncc5[nH]4)c3c2)c1] | 129 |
| 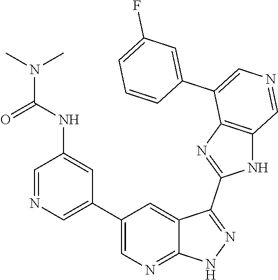 [SMILES: CN(C)C(=O)Nc1cncc(-c2cnc3[nH]nc(-c4nc5c(-c6cccc(F)c6)cncc5[nH]4)c3c2)c1] | 130 |
| 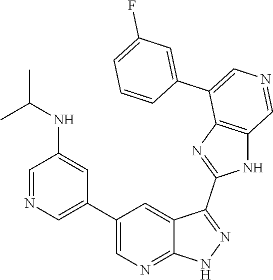 [SMILES: CC(C)Nc1cncc(-c2cnc3[nH]nc(-c4nc5c(-c6cccc(F)c6)cncc5[nH]4)c3c2)c1] | 131 |
| 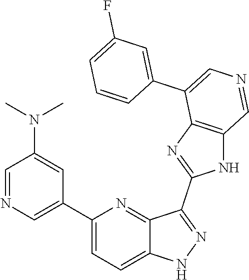 [SMILES: CN(C)c1cncc(-c2ccc3[nH]nc(-c4nc5c(-c6cccc(F)c6)cncc5[nH]4)c3n2)c1] | 132 |
| 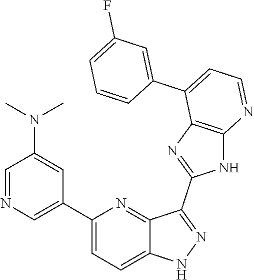 [SMILES: CN(C)c1cncc(-c2ccc3[nH]nc(-c4nc5c(-c6cccc(F)c6)ccnc5[nH]4)c3n2)c1] | 133 |
| 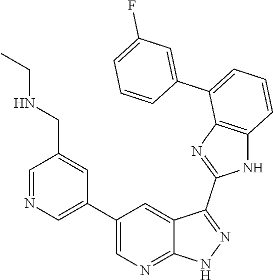 [SMILES: CCNCc1cncc(-c2cnc3[nH]nc(-c4nc5c(-c6cccc(F)c6)cccc5[nH]4)c3c2)c1] | 134 |
| 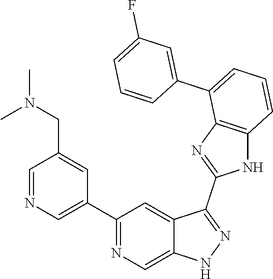 [SMILES: CN(C)Cc1cncc(-c2cc3c(-c4nc5c(-c6cccc(F)c6)cccc5[nH]4)n[nH]c3cn2)c1] | 135 |
| 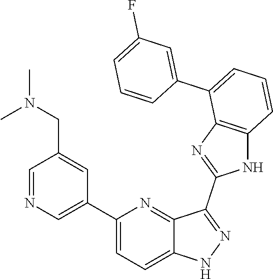 [SMILES: CN(C)Cc1cncc(-c2ccc3[nH]nc(-c4nc5c(-c6cccc(F)c6)cccc5[nH]4)c3n2)c1] | 136 |
| 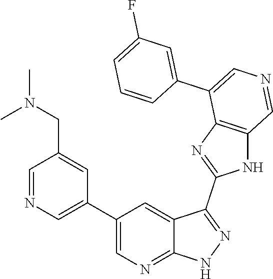 [SMILES: CN(C)Cc1cncc(-c2cnc3[nH]nc(-c4nc5c(-c6cccc(F)c6)cncc5[nH]4)c3c2)c1] | 137 |
| 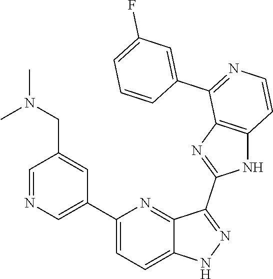 [SMILES: CN(C)Cc1cncc(-c2ccc3[nH]nc(-c4nc5c(-c6cccc(F)c6)nccc5[nH]4)c3n2)c1] | 138 |
| 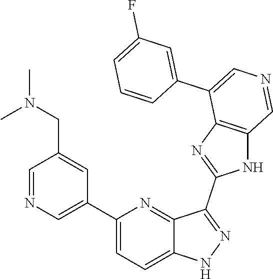 [SMILES: CN(C)Cc1cncc(-c2ccc3[nH]nc(-c4nc5c(-c6cccc(F)c6)cncc5[nH]4)c3n2)c1] | 139 |
| 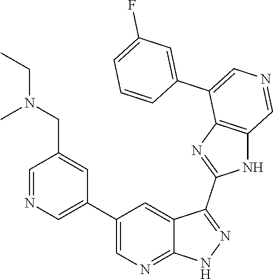 [SMILES: CCN(C)Cc1cncc(-c2cnc3[nH]nc(-c4nc5c(-c6cccc(F)c6)cncc5[nH]4)c3c2)c1] | 140 |
| 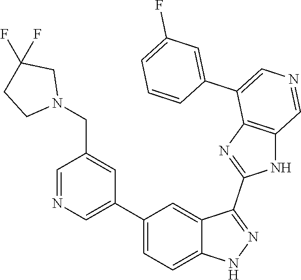 [SMILES: Fc1cccc(-c2cncc3[nH]c(-c4n[nH]c5ccc(-c6cncc(CN7CCC(F)(F)C7)c6)cc45)nc23)c1] | 141 |
| 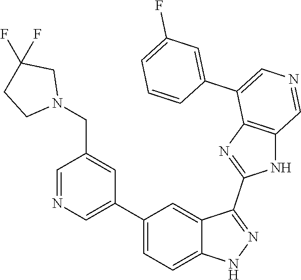 [SMILES: Fc1cccc(-c2cncc3[nH]c(-c4n[nH]c5ccc(-c6cncc(CN7CCC(F)(F)C7)c6)cc45)nc23)c1] | 142 |
| 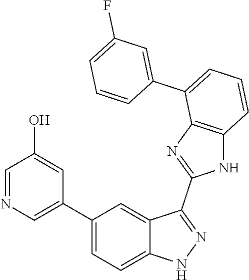 [SMILES: Oc1cncc(-c2ccc3[nH]nc(-c4nc5c(-c6cccc(F)c6)cccc5[nH]4)c3c2)c1] | 143 |
| 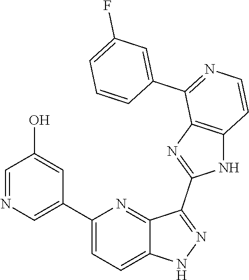 [SMILES: Oc1cncc(-c2ccc3[nH]nc(-c4nc5c(-c6cccc(F)c6)nccc5[nH]4)c3n2)c1] | 144 |
| 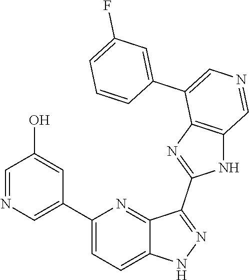 [SMILES: Oc1cncc(-c2ccc3[nH]nc(-c4nc5c(-c6cccc(F)c6)cncc5[nH]4)c3n2)c1] | 145 |
| 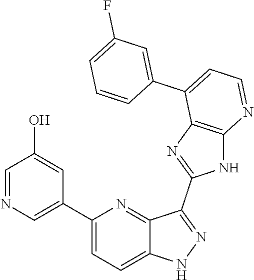 [SMILES: Oc1cncc(-c2ccc3[nH]nc(-c4nc5c(-c6cccc(F)c6)ccnc5[nH]4)c3n2)c1] | 146 |
| 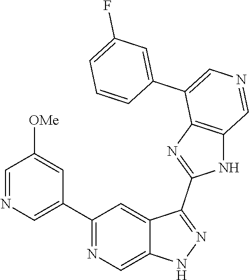 [SMILES: COc1cncc(-c2cc3c(-c4nc5c(-c6cccc(F)c6)cncc5[nH]4)n[nH]c3cn2)c1] | 147 |
| 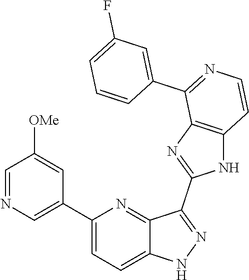 [SMILES: COc1cncc(-c2ccc3[nH]nc(-c4nc5c(-c6cccc(F)c6)nccc5[nH]4)c3n2)c1] | 148 |
| 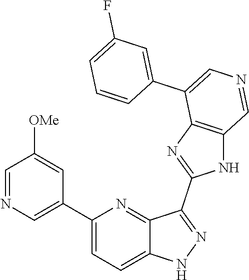 [SMILES: COc1cncc(-c2ccc3[nH]nc(-c4nc5c(-c6cccc(F)c6)cncc5[nH]4)c3n2)c1] | 149 |
| 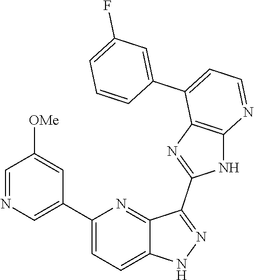 [SMILES: COc1cncc(-c2ccc3[nH]nc(-c4nc5c(-c6cccc(F)c6)ccnc5[nH]4)c3n2)c1] | 150 |
| 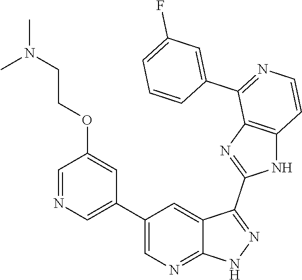 [SMILES: CN(C)CCOc1cncc(-c2cnc3[nH]nc(-c4nc5c(-c6cccc(F)c6)nccc5[nH]4)c3c2)c1] | 151 |
| 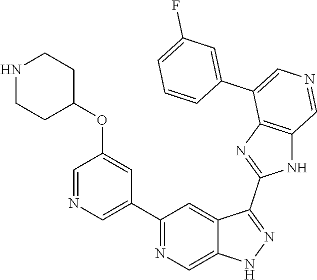 [SMILES: Fc1cccc(-c2cncc3[nH]c(-c4n[nH]c5cnc(-c6cncc(OC7CCNCC7)c6)cc45)nc23)c1] | 152 |
| 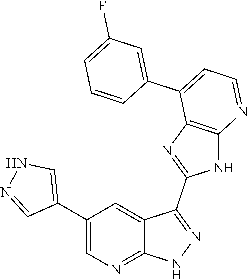 [SMILES: Fc1cccc(-c2ccnc3[nH]c(-c4n[nH]c5ncc(-c6cn[nH]c6)cc45)nc23)c1] | 153 |
| 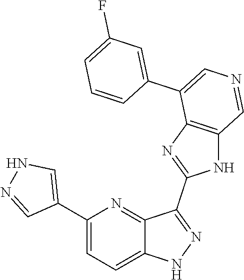 [SMILES: Fc1cccc(-c2cncc3[nH]c(-c4n[nH]c5ccc(-c6cn[nH]c6)nc45)nc23)c1] | 154 |
| 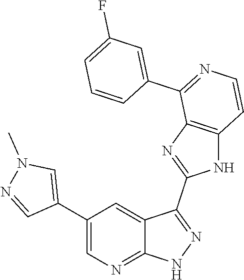 [SMILES: Cn1cc(-c2cnc3[nH]nc(-c4nc5c(-c6cccc(F)c6)nccc5[nH]4)c3c2)cn1] | 155 |
| 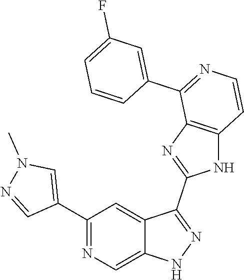 [SMILES: Cn1cc(-c2cc3c(-c4nc5c(-c6cccc(F)c6)nccc5[nH]4)n[nH]c3cn2)cn1] | 156 |
| 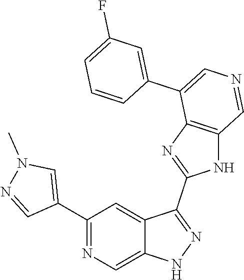 [SMILES: Cn1cc(-c2cc3c(-c4nc5c(-c6cccc(F)c6)cncc5[nH]4)n[nH]c3cn2)cn1] | 157 |
| 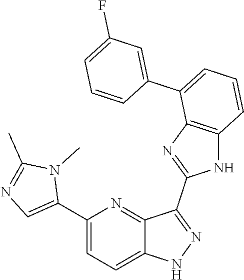 [SMILES: Cc1ncc(-c2ccc3[nH]nc(-c4nc5c(-c6cccc(F)c6)cccc5[nH]4)c3n2)n1C] | 158 |
| 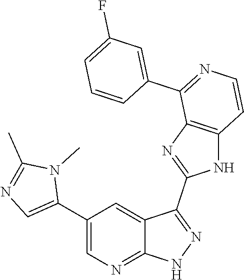 [SMILES: Cc1ncc(-c2cnc3[nH]nc(-c4nc5c(-c6cccc(F)c6)nccc5[nH]4)c3c2)n1C] | 159 |
| 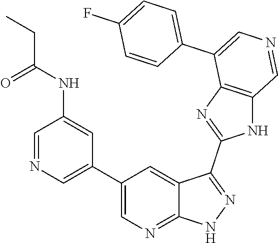 [SMILES: CCC(=O)Nc1cncc(-c2cnc3[nH]nc(-c4nc5c(-c6ccc(F)cc6)cncc5[nH]4)c3c2)c1] | 160 |
| 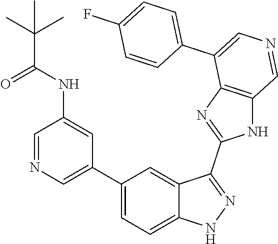 [SMILES: CC(C)(C)C(=O)Nc1cncc(-c2ccc3[nH]nc(-c4nc5c(-c6ccc(F)cc6)cncc5[nH]4)c3c2)c1] | 161 |
| 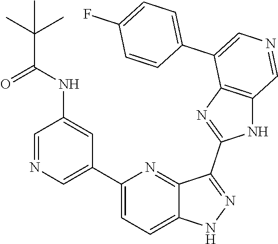 [SMILES: CC(C)(C)C(=O)Nc1cncc(-c2ccc3[nH]nc(-c4nc5c(-c6ccc(F)cc6)cncc5[nH]4)c3n2)c1] | 162 |
| 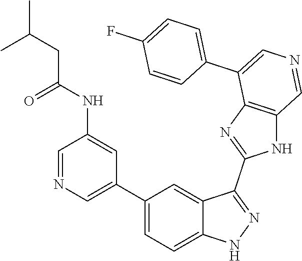 [SMILES: CC(C)CC(=O)Nc1cncc(-c2ccc3[nH]nc(-c4nc5c(-c6ccc(F)cc6)cncc5[nH]4)c3c2)c1] | 163 |
| 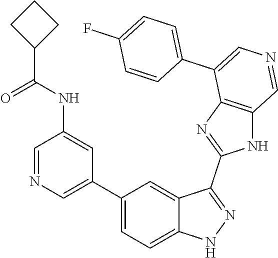 [SMILES: O=C(Nc1cncc(-c2ccc3[nH]nc(-c4nc5c(-c6ccc(F)cc6)cncc5[nH]4)c3c2)c1)C1CCC1] | 164 |
| 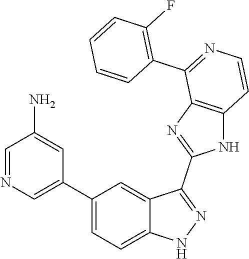 [SMILES: Nc1cncc(-c2ccc3[nH]nc(-c4nc5c(-c6ccccc6F)nccc5[nH]4)c3c2)c1] | 165 |
| 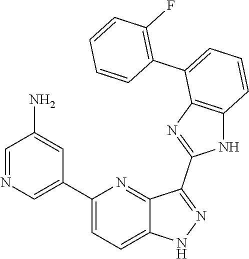 [SMILES: Nc1cncc(-c2ccc3[nH]nc(-c4nc5c(-c6ccccc6F)cccc5[nH]4)c3n2)c1] | 166 |
| 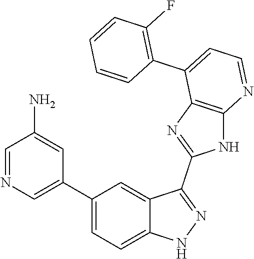 [SMILES: Nc1cncc(-c2ccc3[nH]nc(-c4nc5c(-c6ccccc6F)ccnc5[nH]4)c3c2)c1] | 167 |
| 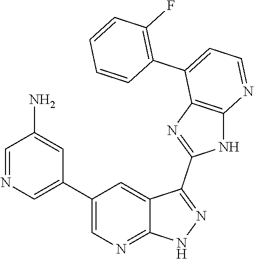 [SMILES: Nc1cncc(-c2cnc3[nH]nc(-c4nc5c(-c6ccccc6F)ccnc5[nH]4)c3c2)c1] | 168 |
| 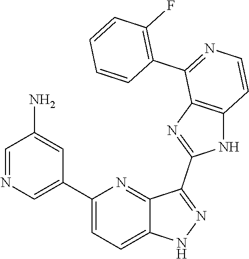 [SMILES: Nc1cncc(-c2ccc3[nH]nc(-c4nc5c(-c6ccccc6F)nccc5[nH]4)c3n2)c1] | 169 |
| 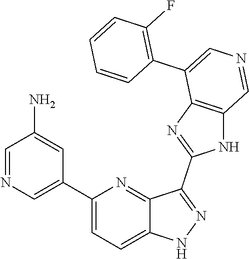 [SMILES: Nc1cncc(-c2ccc3[nH]nc(-c4nc5c(-c6ccccc6F)cncc5[nH]4)c3n2)c1] | 170 |
| 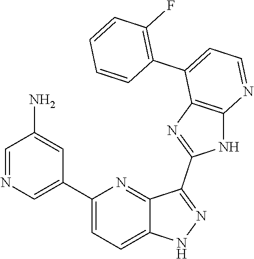 [SMILES: Nc1cncc(-c2ccc3[nH]nc(-c4nc5c(-c6ccccc6F)ccnc5[nH]4)c3n2)c1] | 171 |
| 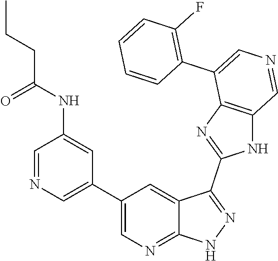 [SMILES: CCCC(=O)Nc1cncc(-c2cnc3[nH]nc(-c4nc5c(-c6ccccc6F)cncc5[nH]4)c3c2)c1] | 172 |
| 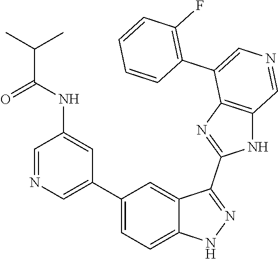 [SMILES: CC(C)C(=O)Nc1cncc(-c2ccc3[nH]nc(-c4nc5c(-c6ccccc6F)cncc5[nH]4)c3c2)c1] | 173 |
| 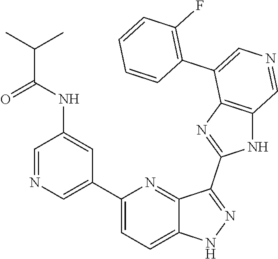 [SMILES: CC(C)C(=O)Nc1cncc(-c2ccc3[nH]nc(-c4nc5c(-c6ccccc6F)cncc5[nH]4)c3n2)c1] | 174 |
| 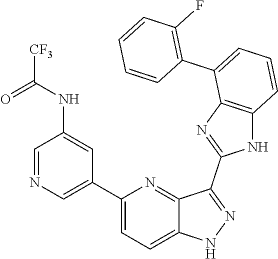 [SMILES: O=C(Nc1cncc(-c2ccc3[nH]nc(-c4nc5c(-c6ccccc6F)cccc5[nH]4)c3n2)c1)C(F)(F)F] | 175 |
| 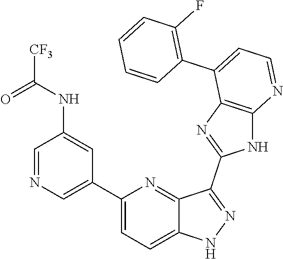 [SMILES: O=C(Nc1cncc(-c2ccc3[nH]nc(-c4nc5c(-c6ccccc6F)ccnc5[nH]4)c3n2)c1)C(F)(F)F] | 176 |
| 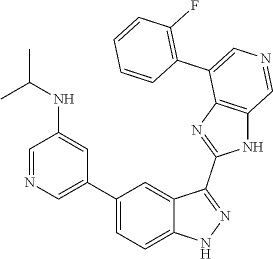 [SMILES: CC(C)Nc1cncc(-c2ccc3[nH]nc(-c4nc5c(-c6ccccc6F)cncc5[nH]4)c3c2)c1] | 177 |
| 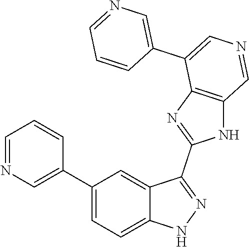 [SMILES: c1cncc(-c2ccc3[nH]nc(-c4nc5c(-c6cccnc6)cncc5[nH]4)c3c2)c1] | 178 |
| 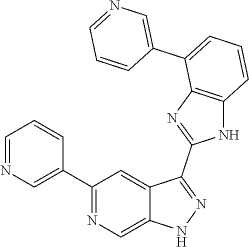 [SMILES: c1cncc(-c2cc3c(-c4nc5c(-c6cccnc6)cccc5[nH]4)n[nH]c3cn2)c1] | 179 |
| 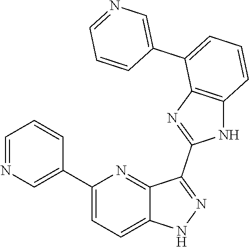 [SMILES: c1cncc(-c2ccc3[nH]nc(-c4nc5c(-c6cccnc6)cccc5[nH]4)c3n2)c1] | 180 |
| 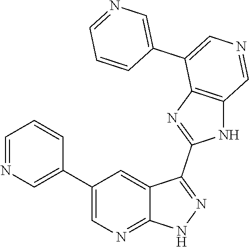 [SMILES: c1cncc(-c2cnc3[nH]nc(-c4nc5c(-c6cccnc6)cncc5[nH]4)c3c2)c1] | 181 |
| 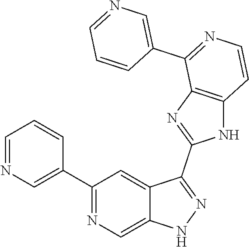 [SMILES: c1cncc(-c2cc3c(-c4nc5c(-c6cccnc6)nccc5[nH]4)n[nH]c3cn2)c1] | 182 |
| 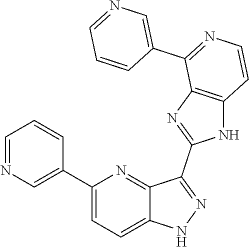 [SMILES: c1cncc(-c2ccc3[nH]nc(-c4nc5c(-c6cccnc6)nccc5[nH]4)c3n2)c1] | 183 |
| 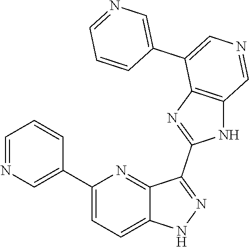 [SMILES: c1cncc(-c2ccc3[nH]nc(-c4nc5c(-c6cccnc6)cncc5[nH]4)c3n2)c1] | 184 |
| 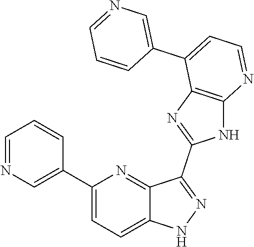 [SMILES: c1cncc(-c2ccc3[nH]nc(-c4nc5c(-c6cccnc6)ccnc5[nH]4)c3n2)c1] | 185 |
| 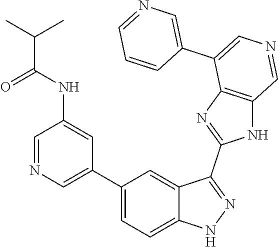 [SMILES: CC(C)C(=O)Nc1cncc(-c2ccc3[nH]nc(-c4nc5c(-c6cccnc6)cncc5[nH]4)c3c2)c1] | 186 |
| 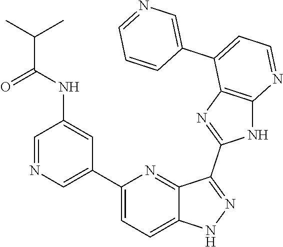 [SMILES: CC(C)C(=O)Nc1cncc(-c2ccc3[nH]nc(-c4nc5c(-c6cccnc6)ccnc5[nH]4)c3n2)c1] | 187 |
| 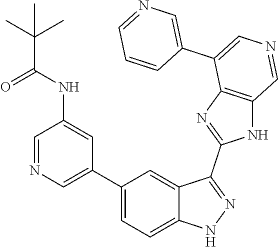 [SMILES: CC(C)(C)C(=O)Nc1cncc(-c2ccc3[nH]nc(-c4nc5c(-c6cccnc6)cncc5[nH]4)c3c2)c1] | 188 |
| 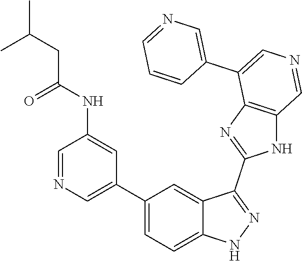 [SMILES: CC(C)CC(=O)Nc1cncc(-c2ccc3[nH]nc(-c4nc5c(-c6cccnc6)cncc5[nH]4)c3c2)c1] | 189 |
| 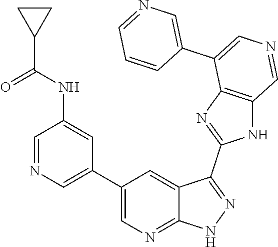 [SMILES: O=C(Nc1cncc(-c2cnc3[nH]nc(-c4nc5c(-c6cccnc6)cncc5[nH]4)c3c2)c1)C1CC1] | 190 |
| 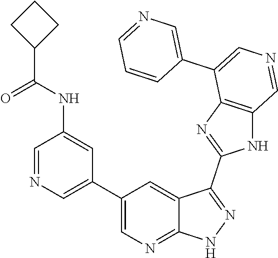 [SMILES: O=C(Nc1cncc(-c2cnc3[nH]nc(-c4nc5c(-c6cccnc6)cncc5[nH]4)c3c2)c1)C1CCC1] | 191 |
| 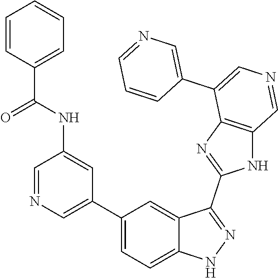 [SMILES: O=C(Nc1cncc(-c2ccc3[nH]nc(-c4nc5c(-c6cccnc6)cncc5[nH]4)c3c2)c1)c1ccccc1] | 192 |
| 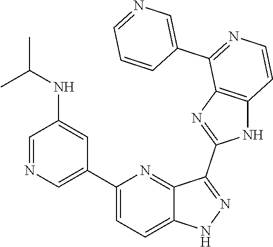 [SMILES: CC(C)Nc1cncc(-c2ccc3[nH]nc(-c4nc5c(-c6cccnc6)nccc5[nH]4)c3n2)c1] | 193 |
| 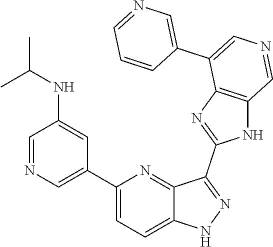 [SMILES: CC(C)Nc1cncc(-c2ccc3[nH]nc(-c4nc5c(-c6cccnc6)cncc5[nH]4)c3n2)c1] | 194 |
| 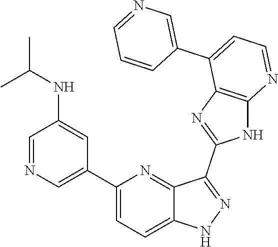 [SMILES: CC(C)Nc1cncc(-c2ccc3[nH]nc(-c4nc5c(-c6cccnc6)ccnc5[nH]4)c3n2)c1] | 195 |
| 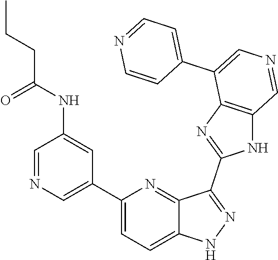 [SMILES: CCCC(=O)Nc1cncc(-c2ccc3[nH]nc(-c4nc5c(-c6ccncc6)cncc5[nH]4)c3n2)c1] | 196 |
| 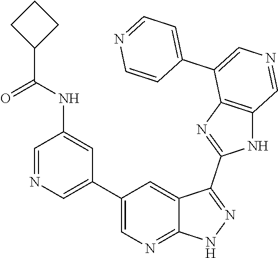 [SMILES: O=C(Nc1cncc(-c2cnc3[nH]nc(-c4nc5c(-c6ccncc6)cncc5[nH]4)c3c2)c1)C1CCC1] | 197 |
| 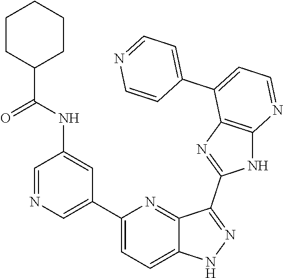 [SMILES: O=C(Nc1cncc(-c2ccc3[nH]nc(-c4nc5c(-c6ccncc6)ccnc5[nH]4)c3n2)c1)C1CCCCC1] | 198 |
| 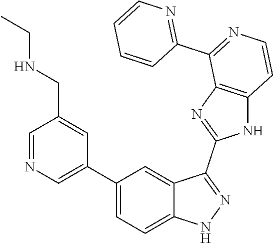 [SMILES: CCNCc1cncc(-c2ccc3[nH]nc(-c4nc5c(-c6ccccn6)nccc5[nH]4)c3c2)c1] | 199 |
| 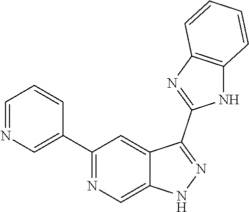 [SMILES: c1cncc(-c2cc3c(-c4nc5ccccc5[nH]4)n[nH]c3cn2)c1] | 200 |
| 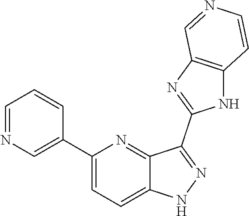 [SMILES: c1cncc(-c2ccc3[nH]nc(-c4nc5cnccc5[nH]4)c3n2)c1] | 201 |
| 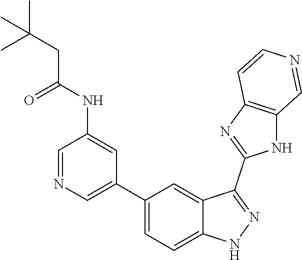 [SMILES: CC(C)(C)CC(=O)Nc1cncc(-c2ccc3[nH]nc(-c4nc5ccncc5[nH]4)c3c2)c1] | 202 |
| 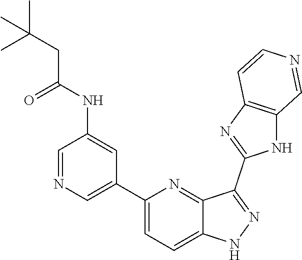 [SMILES: CC(C)(C)CC(=O)Nc1cncc(-c2ccc3[nH]nc(-c4nc5ccncc5[nH]4)c3n2)c1] | 203 |
| 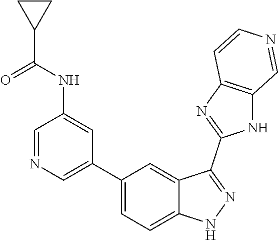 [SMILES: O=C(Nc1cncc(-c2ccc3[nH]nc(-c4nc5ccncc5[nH]4)c3c2)c1)C1CC1] | 204 |
| 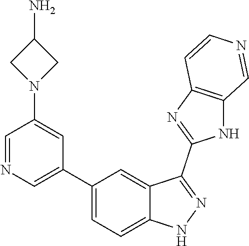 [SMILES: NC1CN(c2cncc(-c3ccc4[nH]nc(-c5nc6ccncc6[nH]5)c4c3)c2)C1] | 205 |
| 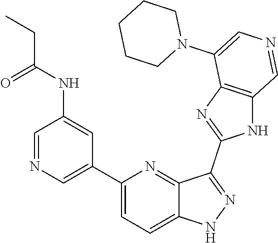 [SMILES: CCC(=O)Nc1cncc(-c2ccc3[nH]nc(-c4nc5c(N6CCCCC6)cncc5[nH]4)c3n2)c1] | 206 |
| 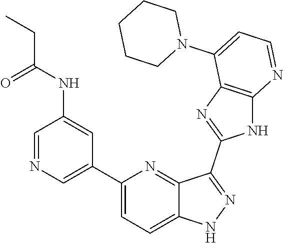 [SMILES: CCC(=O)Nc1cncc(-c2ccc3[nH]nc(-c4nc5c(N6CCCCC6)ccnc5[nH]4)c3n2)c1] | 207 |
| 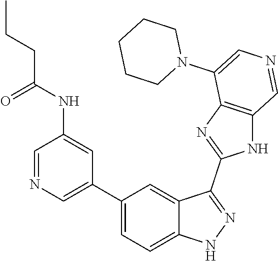 [SMILES: CCCC(=O)Nc1cncc(-c2ccc3[nH]nc(-c4nc5c(N6CCCCC6)cncc5[nH]4)c3c2)c1] | 208 |
| 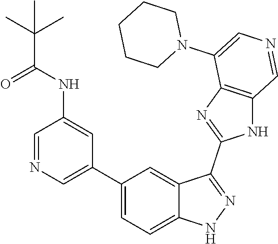 [SMILES: CC(C)(C)C(=O)Nc1cncc(-c2ccc3[nH]nc(-c4nc5c(N6CCCCC6)cncc5[nH]4)c3c2)c1] | 209 |
| 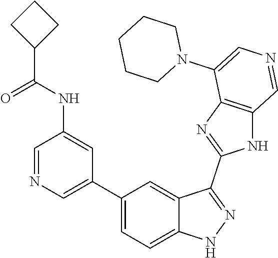 [SMILES: O=C(Nc1cncc(-c2ccc3[nH]nc(-c4nc5c(N6CCCCC6)cncc5[nH]4)c3c2)c1)C1CCC1] | 210 |
| 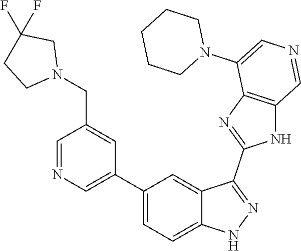 [SMILES: FC1(F)CCN(Cc2cncc(-c3ccc4[nH]nc(-c5nc6c(N7CCCCC7)cncc6[nH]5)c4c3)c2)C1] | 211 |
| 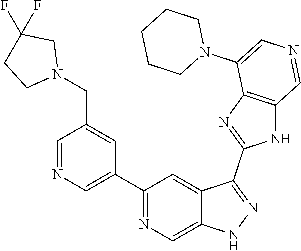 [SMILES: FC1(F)CCN(Cc2cncc(-c3cc4c(-c5nc6c(N7CCCCC7)cncc6[nH]5)n[nH]c4cn3)c2)C1] | 212 |
| 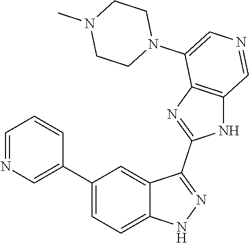 [SMILES: CN1CCN(c2cncc3[nH]c(-c4n[nH]c5ccc(-c6cccnc6)cc45)nc23)CC1] | 213 |
| 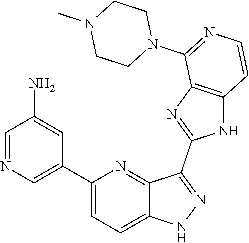 [SMILES: CN1CCN(c2nccc3[nH]c(-c4n[nH]c5ccc(-c6cncc(N)c6)nc45)nc23)CC1] | 214 |
| 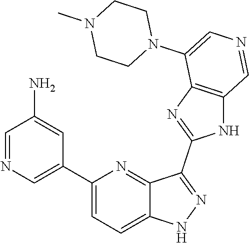 [SMILES: CN1CCN(c2cncc3[nH]c(-c4n[nH]c5ccc(-c6cncc(N)c6)nc45)nc23)CC1] | 215 |
| 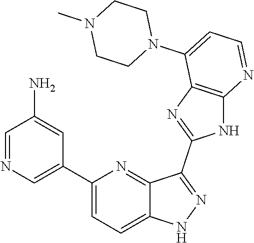 [SMILES: CN1CCN(c2ccnc3[nH]c(-c4n[nH]c5ccc(-c6cncc(N)c6)nc45)nc23)CC1] | 216 |
| 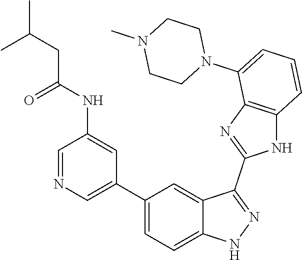 [SMILES: CC(C)CC(=O)Nc1cncc(-c2ccc3[nH]nc(-c4nc5c(N6CCN(C)CC6)cccc5[nH]4)c3c2)c1] | 217 |
| 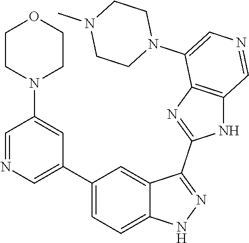 [SMILES: CN1CCN(c2cncc3[nH]c(-c4n[nH]c5ccc(-c6cncc(N7CCOCC7)c6)cc45)nc23)CC1] | 218 |
| 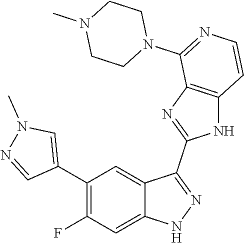 [SMILES: CN1CCN(c2nccc3[nH]c(-c4n[nH]c5cc(F)c(-c6cnn(C)c6)cc45)nc23)CC1] | 219 |
| 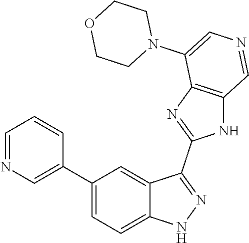 [SMILES: c1cncc(-c2ccc3[nH]nc(-c4nc5c(N6CCOCC6)cncc5[nH]4)c3c2)c1] | 220 |
| 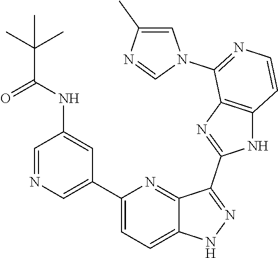 [SMILES: Cc1cn(-c2nccc3[nH]c(-c4n[nH]c5ccc(-c6cncc(NC(=O)C(C)(C)C)c6)nc45)nc23)cn1] | 221 |
| 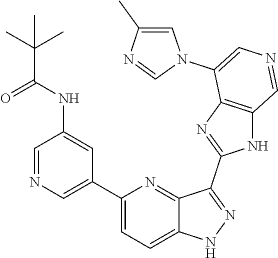 [SMILES: Cc1cn(-c2cncc3[nH]c(-c4n[nH]c5ccc(-c6cncc(NC(=O)C(C)(C)C)c6)nc45)nc23)cn1] | 222 |
| 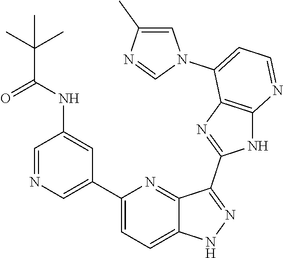 [SMILES: Cc1cn(-c2ccnc3[nH]c(-c4n[nH]c5ccc(-c6cncc(NC(=O)C(C)(C)C)c6)nc45)nc23)cn1] | 223 |
| 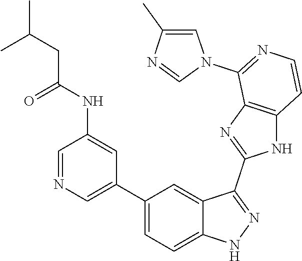 [SMILES: Cc1cn(-c2nccc3[nH]c(-c4n[nH]c5ccc(-c6cncc(NC(=O)CC(C)C)c6)cc45)nc23)cn1] | 224 |
| 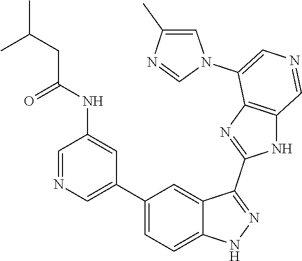 [SMILES: Cc1cn(-c2cncc3[nH]c(-c4n[nH]c5ccc(-c6cncc(NC(=O)CC(C)C)c6)cc45)nc23)cn1] | 225 |
| 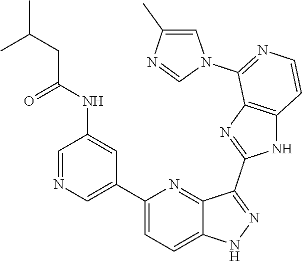 [SMILES: Cc1cn(-c2nccc3[nH]c(-c4n[nH]c5ccc(-c6cncc(NC(=O)CC(C)C)c6)nc45)nc23)cn1] | 226 |
| 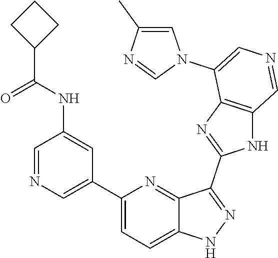 [SMILES: Cc1cn(-c2cncc3[nH]c(-c4n[nH]c5ccc(-c6cncc(NC(=O)C7CCC7)c6)nc45)nc23)cn1] | 227 |
| 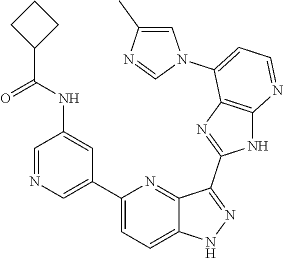 [SMILES: Cc1cn(-c2ccnc3[nH]c(-c4n[nH]c5ccc(-c6cncc(NC(=O)C7CCC7)c6)nc45)nc23)cn1] | 228 |
| 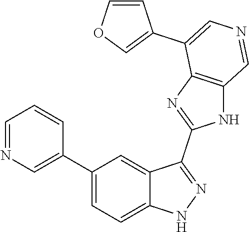 [SMILES: c1cncc(-c2ccc3[nH]nc(-c4nc5c(-c6ccoc6)cncc5[nH]4)c3c2)c1] | 229 |
| 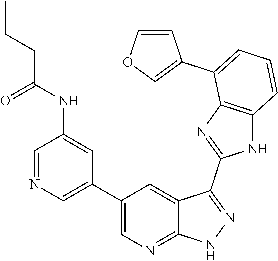 [SMILES: CCCC(=O)Nc1cncc(-c2cnc3[nH]nc(-c4nc5c(-c6ccoc6)cccc5[nH]4)c3c2)c1] | 230 |
| 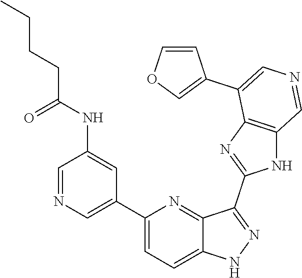 [SMILES: CCCCC(=O)Nc1cncc(-c2ccc3[nH]nc(-c4nc5c(-c6ccoc6)cncc5[nH]4)c3n2)c1] | 231 |
| 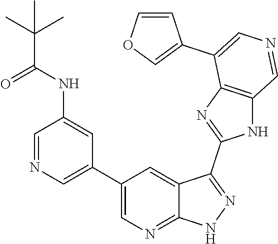 [SMILES: CC(C)(C)C(=O)Nc1cncc(-c2cnc3[nH]nc(-c4nc5c(-c6ccoc6)cncc5[nH]4)c3c2)c1] | 232 |
| 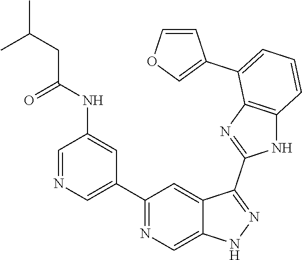 [SMILES: CC(C)CC(=O)Nc1cncc(-c2cc3c(-c4nc5c(-c6ccoc6)cccc5[nH]4)n[nH]c3cn2)c1] | 233 |
| 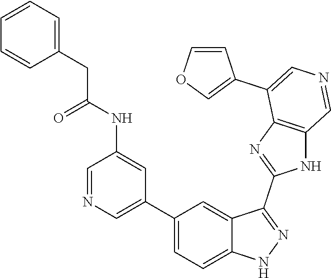 [SMILES: O=C(Cc1ccccc1)Nc1cncc(-c2ccc3[nH]nc(-c4nc5c(-c6ccoc6)cncc5[nH]4)c3c2)c1] | 234 |
| 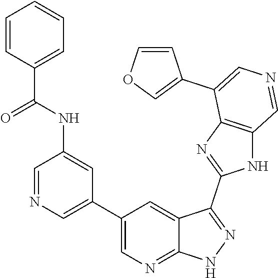 [SMILES: O=C(Nc1cncc(-c2cnc3[nH]nc(-c4nc5c(-c6ccoc6)cncc5[nH]4)c3c2)c1)c1ccccc1] | 235 |
| 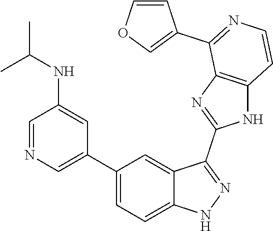 [SMILES: CC(C)Nc1cncc(-c2ccc3[nH]nc(-c4nc5c(-c6ccoc6)nccc5[nH]4)c3c2)c1] | 236 |
| 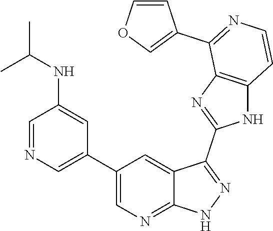 [SMILES: CC(C)Nc1cncc(-c2cnc3[nH]nc(-c4nc5c(-c6ccoc6)nccc5[nH]4)c3c2)c1] | 237 |
| 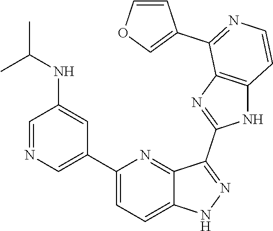 [SMILES: CC(C)Nc1cncc(-c2ccc3[nH]nc(-c4nc5c(-c6ccoc6)nccc5[nH]4)c3n2)c1] | 238 |
| 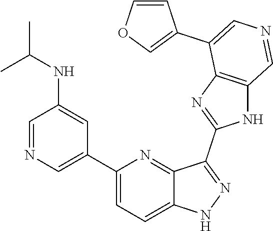 [SMILES: CC(C)Nc1cncc(-c2ccc3[nH]nc(-c4nc5c(-c6ccoc6)cncc5[nH]4)c3n2)c1] | 239 |
| 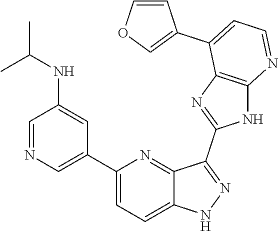 [SMILES: CC(C)Nc1cncc(-c2ccc3[nH]nc(-c4nc5c(-c6ccoc6)ccnc5[nH]4)c3n2)c1] | 240 |
| 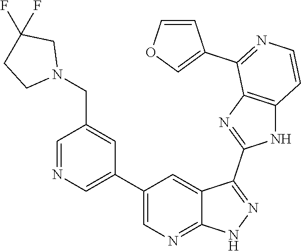 [SMILES: FC1(F)CCN(Cc2cncc(-c3cnc4[nH]nc(-c5nc6c(-c7ccoc7)nccc6[nH]5)c4c3)c2)C1] | 241 |
| 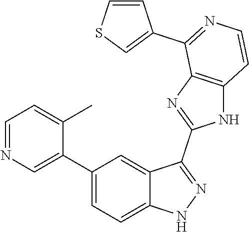 [SMILES: Cc1ccncc1-c1ccc2[nH]nc(-c3nc4c(-c5ccsc5)nccc4[nH]3)c2c1] | 242 |
| 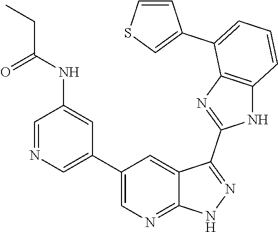 [SMILES: CCC(=O)Nc1cncc(-c2cnc3[nH]nc(-c4nc5c(-c6ccsc6)cccc5[nH]4)c3c2)c1] | 243 |
| 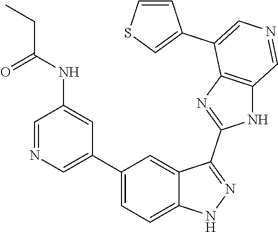 [SMILES: CCC(=O)Nc1cncc(-c2ccc3[nH]nc(-c4nc5c(-c6ccsc6)cncc5[nH]4)c3c2)c1] | 244 |
| 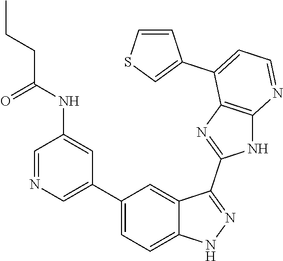 [SMILES: CCCC(=O)Nc1cncc(-c2ccc3[nH]nc(-c4nc5c(-c6ccsc6)ccnc5[nH]4)c3c2)c1] | 245 |
| 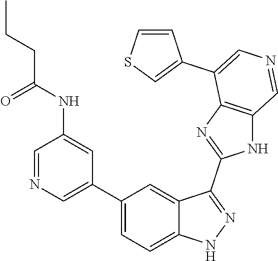 [SMILES: CCCC(=O)Nc1cncc(-c2ccc3[nH]nc(-c4nc5c(-c6ccsc6)cncc5[nH]4)c3c2)c1] | 246 |
| 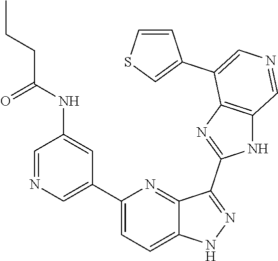 [SMILES: CCCC(=O)Nc1cncc(-c2ccc3[nH]nc(-c4nc5c(-c6ccsc6)cncc5[nH]4)c3n2)c1] | 247 |
| 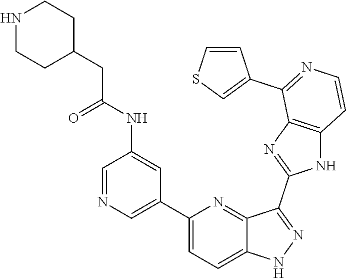 [SMILES: O=C(CC1CCNCC1)Nc1cncc(-c2ccc3[nH]nc(-c4nc5c(-c6ccsc6)nccc5[nH]4)c3n2)c1] | 248 |
| 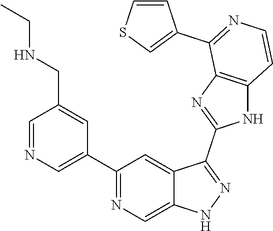 [SMILES: CCNCc1cncc(-c2cc3c(-c4nc5c(-c6ccsc6)nccc5[nH]4)n[nH]c3cn2)c1] | 249 |
| 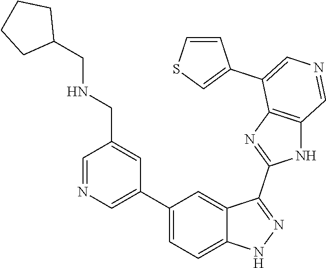 [SMILES: c1cc(-c2cncc3[nH]c(-c4n[nH]c5ccc(-c6cncc(CNCC7CCCC7)c6)cc45)nc23)cs1] | 250 |
| 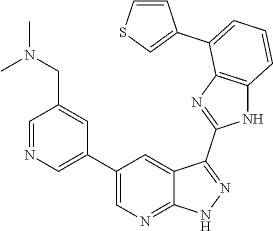 [SMILES: CN(C)Cc1cncc(-c2cnc3[nH]nc(-c4nc5c(-c6ccsc6)cccc5[nH]4)c3c2)c1] | 251 |
| 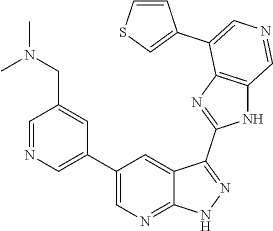 [SMILES: CN(C)Cc1cncc(-c2cnc3[nH]nc(-c4nc5c(-c6ccsc6)cncc5[nH]4)c3c2)c1] | 252 |
| 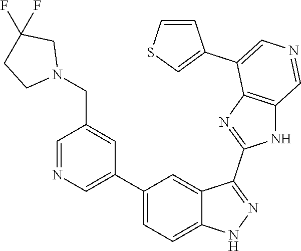 [SMILES: FC1(F)CCN(Cc2cncc(-c3ccc4[nH]nc(-c5nc6c(-c7ccsc7)cncc6[nH]5)c4c3)c2)C1] | 253 |
| 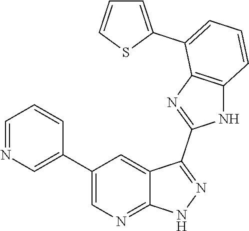 [SMILES: c1cncc(-c2cnc3[nH]nc(-c4nc5c(-c6cccs6)cccc5[nH]4)c3c2)c1] | 254 |
| 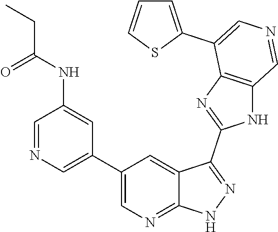 [SMILES: CCC(=O)Nc1cncc(-c2cnc3[nH]nc(-c4nc5c(-c6cccs6)cncc5[nH]4)c3c2)c1] | 255 |
| 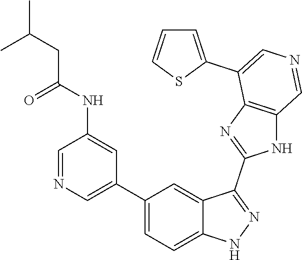 [SMILES: CC(C)CC(=O)Nc1cncc(-c2ccc3[nH]nc(-c4nc5c(-c6cccs6)cncc5[nH]4)c3c2)c1] | 256 |
| 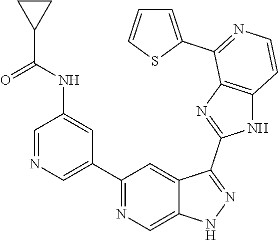 [SMILES: O=C(Nc1cncc(-c2cc3c(-c4nc5c(-c6cccs6)nccc5[nH]4)n[nH]c3cn2)c1)C1CC1] | 257 |
| 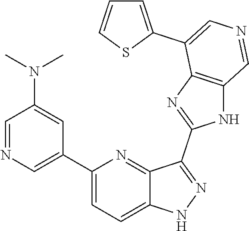 [SMILES: CN(C)c1cncc(-c2ccc3[nH]nc(-c4nc5c(-c6cccs6)cncc5[nH]4)c3n2)c1] | 258 |
| 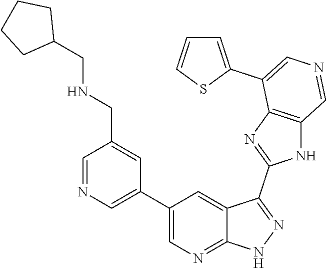 [SMILES: c1csc(-c2cncc3[nH]c(-c4n[nH]c5ncc(-c6cncc(CNCC7CCCC7)c6)cc45)nc23)c1] | 259 |
| 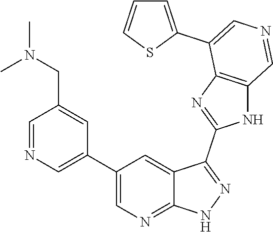 [SMILES: CN(C)Cc1cncc(-c2cnc3[nH]nc(-c4nc5c(-c6cccs6)cncc5[nH]4)c3c2)c1] | 260 |
| 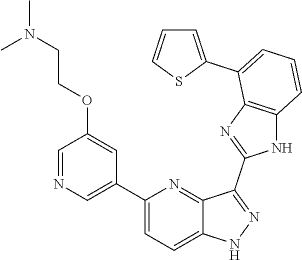 [SMILES: CN(C)CCOc1cncc(-c2ccc3[nH]nc(-c4nc5c(-c6cccs6)cccc5[nH]4)c3n2)c1] | 261 |
| 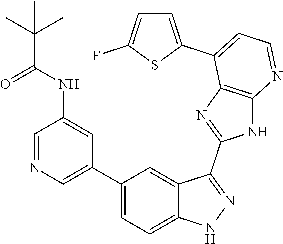 [SMILES: CC(C)(C)C(=O)Nc1cncc(-c2ccc3[nH]nc(-c4nc5c(-c6ccc(F)s6)ccnc5[nH]4)c3c2)c1] | 262 |
| 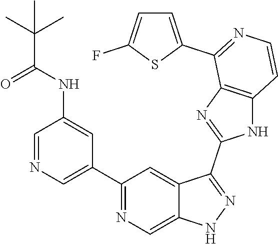 [SMILES: CC(C)(C)C(=O)Nc1cncc(-c2cc3c(-c4nc5c(-c6ccc(F)s6)nccc5[nH]4)n[nH]c3cn2)c1] | 263 |
| 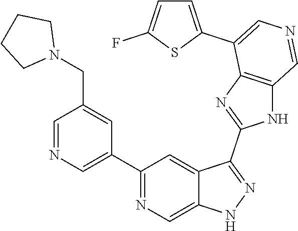 [SMILES: Fc1ccc(-c2cncc3[nH]c(-c4n[nH]c5cnc(-c6cncc(CN7CCCC7)c6)cc45)nc23)s1] | 264 |
| 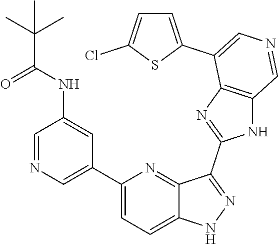 [SMILES: CC(C)(C)C(=O)Nc1cncc(-c2ccc3[nH]nc(-c4nc5c(-c6ccc(Cl)s6)cncc5[nH]4)c3n2)c1] | 265 |
| 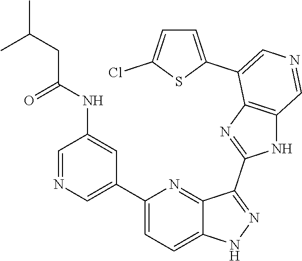 [SMILES: CC(C)CC(=O)Nc1cncc(-c2ccc3[nH]nc(-c4nc5c(-c6ccc(Cl)s6)cncc5[nH]4)c3n2)c1] | 266 |
| 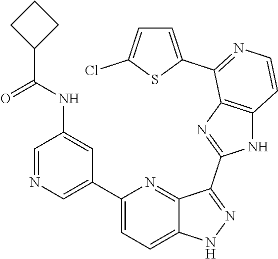 [SMILES: O=C(Nc1cncc(-c2ccc3[nH]nc(-c4nc5c(-c6ccc(Cl)s6)nccc5[nH]4)c3n2)c1)C1CCC1] | 267 |
| 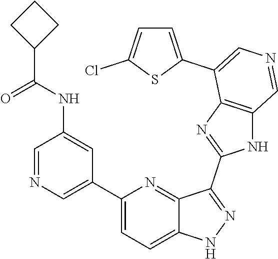 [SMILES: O=C(Nc1cncc(-c2ccc3[nH]nc(-c4nc5c(-c6ccc(Cl)s6)cncc5[nH]4)c3n2)c1)C1CCC1] | 268 |
| 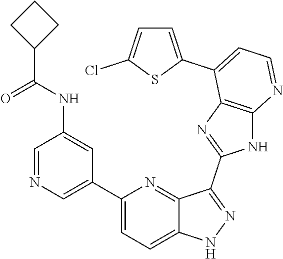 [SMILES: O=C(Nc1cncc(-c2ccc3[nH]nc(-c4nc5c(-c6ccc(Cl)s6)ccnc5[nH]4)c3n2)c1)C1CCC1] | 269 |
| 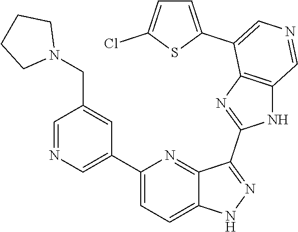 [SMILES: Clc1ccc(-c2cncc3[nH]c(-c4n[nH]c5ccc(-c6cncc(CN7CCCC7)c6)nc45)nc23)s1] | 270 |
| 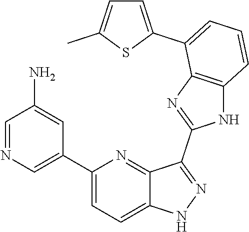 [SMILES: Cc1ccc(-c2cccc3[nH]c(-c4n[nH]c5ccc(-c6cncc(N)c6)nc45)nc23)s1] | 271 |
| 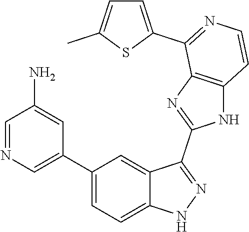 [SMILES: Cc1ccc(-c2nccc3[nH]c(-c4n[nH]c5ccc(-c6cncc(N)c6)cc45)nc23)s1] | 272 |
| 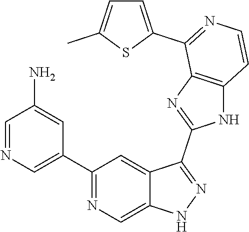 [SMILES: Cc1ccc(-c2nccc3[nH]c(-c4n[nH]c5cnc(-c6cncc(N)c6)cc45)nc23)s1] | 273 |
| 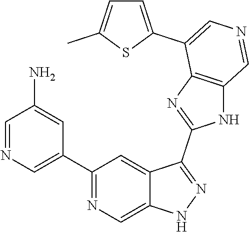 [SMILES: Cc1ccc(-c2cncc3[nH]c(-c4n[nH]c5cnc(-c6cncc(N)c6)cc45)nc23)s1] | 274 |
| 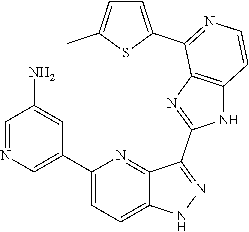 [SMILES: Cc1ccc(-c2nccc3[nH]c(-c4n[nH]c5ccc(-c6cncc(N)c6)nc45)nc23)s1] | 275 |
| 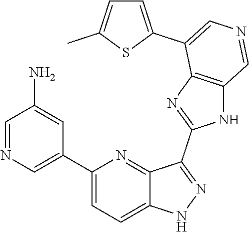 [SMILES: Cc1ccc(-c2cncc3[nH]c(-c4n[nH]c5ccc(-c6cncc(N)c6)nc45)nc23)s1] | 276 |
| 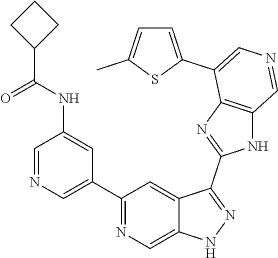 [SMILES: Cc1ccc(-c2cncc3[nH]c(-c4n[nH]c5cnc(-c6cncc(NC(=O)C7CCC7)c6)cc45)nc23)s1] | 277 |
| 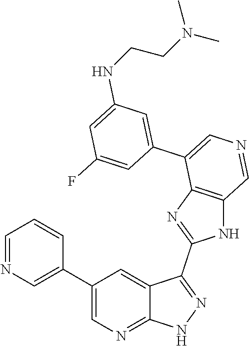 [SMILES: CN(C)CCNc1cc(F)cc(-c2cncc3[nH]c(-c4n[nH]c5ncc(-c6cccnc6)cc45)nc23)c1] | 278 |
| 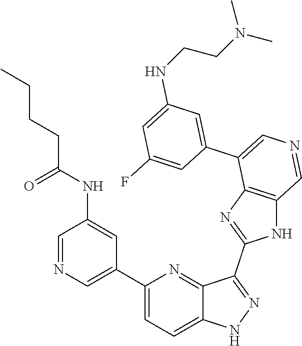 [SMILES: CCCCC(=O)Nc1cncc(-c2ccc3[nH]nc(-c4nc5c(-c6cc(F)cc(NCCN(C)C)c6)cncc5[nH]4)c3n2)c1] | 279 |
| 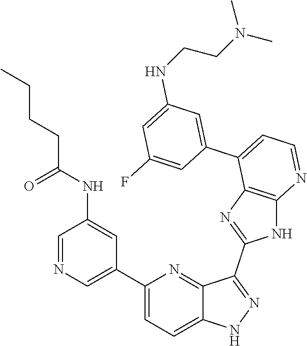 [SMILES: CCCCC(=O)Nc1cncc(-c2ccc3[nH]nc(-c4nc5c(-c6cc(F)cc(NCCN(C)C)c6)ccnc5[nH]4)c3n2)c1] | 280 |
| 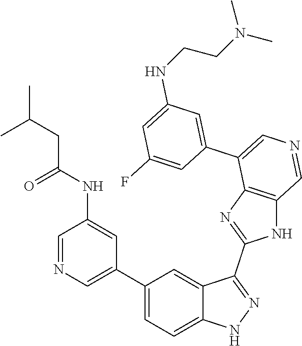 [SMILES: CC(C)CC(=O)Nc1cncc(-c2ccc3[nH]nc(-c4nc5c(-c6cc(F)cc(NCCN(C)C)c6)cncc5[nH]4)c3c2)c1] | 281 |
| 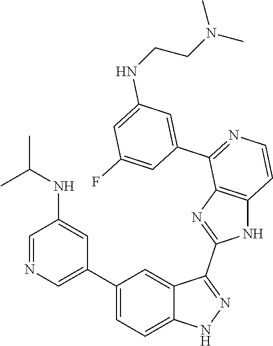 [SMILES: CC(C)Nc1cncc(-c2ccc3[nH]nc(-c4nc5c(-c6cc(F)cc(NCCN(C)C)c6)nccc5[nH]4)c3c2)c1] | 282 |
| 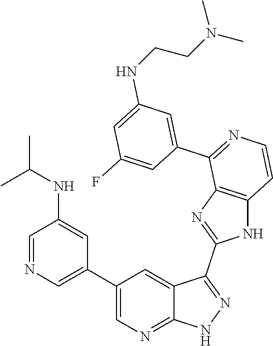 [SMILES: CC(C)Nc1cncc(-c2cnc3[nH]nc(-c4nc5c(-c6cc(F)cc(NCCN(C)C)c6)nccc5[nH]4)c3c2)c1] | 283 |
| 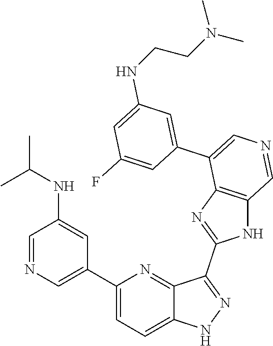 [SMILES: CC(C)Nc1cncc(-c2ccc3[nH]nc(-c4nc5c(-c6cc(F)cc(NCCN(C)C)c6)cncc5[nH]4)c3n2)c1] | 274 |
| 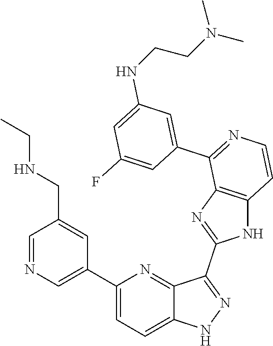 [SMILES: CCNCc1cncc(-c2ccc3[nH]nc(-c4nc5c(-c6cc(F)cc(NCCN(C)C)c6)nccc5[nH]4)c3n2)c1] | 285 |
| 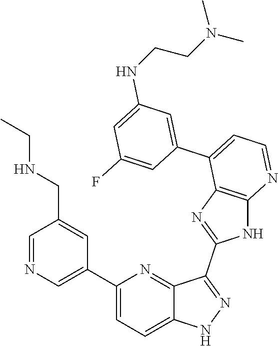 [SMILES: CCNCc1cncc(-c2ccc3[nH]nc(-c4nc5c(-c6cc(F)cc(NCCN(C)C)c6)ccnc5[nH]4)c3n2)c1] | 286 |
| 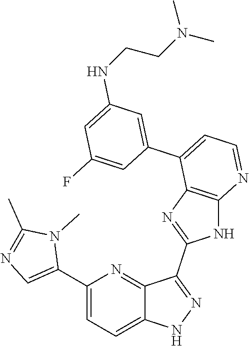 [SMILES: Cc1ncc(-c2ccc3[nH]nc(-c4nc5c(-c6cc(F)cc(NCCN(C)C)c6)ccnc5[nH]4)c3n2)n1C] | 287 |
| 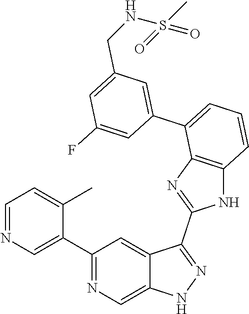 [SMILES: Cc1ccncc1-c1cc2c(-c3nc4c(-c5cc(F)cc(CNS(C)(=O)=O)c5)cccc4[nH]3)n[nH]c2cn1] | 288 |
| 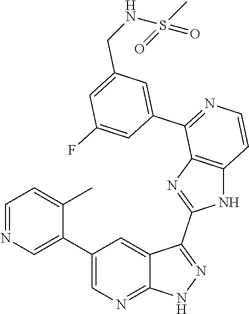 [SMILES: Cc1ccncc1-c1cnc2[nH]nc(-c3nc4c(-c5cc(F)cc(CNS(C)(=O)=O)c5)nccc4[nH]3)c2c1] | 289 |
| 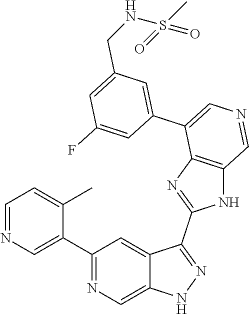 [SMILES: Cc1ccncc1-c1cc2c(-c3nc4c(-c5cc(F)cc(CNS(C)(=O)=O)c5)cncc4[nH]3)n[nH]c2cn1] | 290 |
| 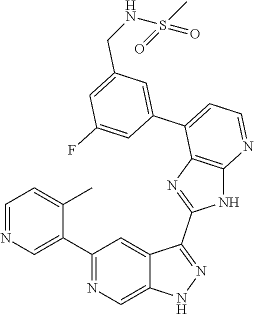 [SMILES: Cc1ccncc1-c1cc2c(-c3nc4c(-c5cc(F)cc(CNS(C)(=O)=O)c5)ccnc4[nH]3)n[nH]c2cn1] | 291 |
| 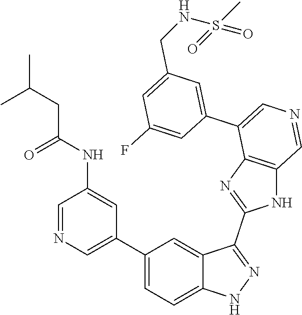 [SMILES: CC(C)CC(=O)Nc1cncc(-c2ccc3[nH]nc(-c4nc5c(-c6cc(F)cc(CNS(C)(=O)=O)c6)cncc5[nH]4)c3c2)c1] | 292 |
| 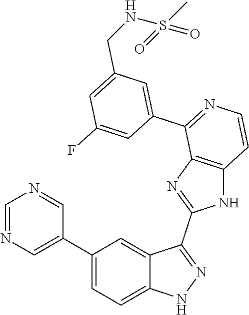 [SMILES: CS(=O)(=O)NCc1cc(F)cc(-c2nccc3[nH]c(-c4n[nH]c5ccc(-c6cncnc6)cc45)nc23)c1] | 293 |
| 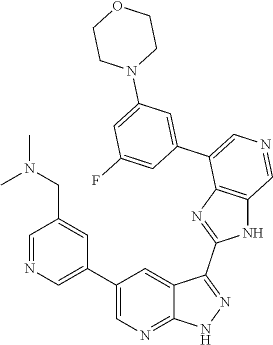 [SMILES: CN(C)Cc1cncc(-c2cnc3[nH]nc(-c4nc5c(-c6cc(F)cc(N7CCOCC7)c6)cncc5[nH]4)c3c2)c1] | 294 |
| 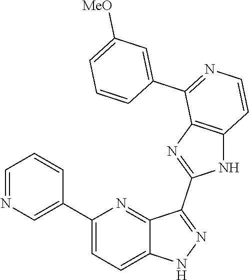 [SMILES: COc1cccc(-c2nccc3[nH]c(-c4n[nH]c5ccc(-c6cccnc6)nc45)nc23)c1] | 295 |
| 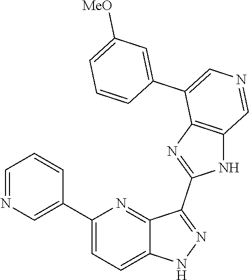 [SMILES: COc1cccc(-c2cncc3[nH]c(-c4n[nH]c5ccc(-c6cccnc6)nc45)nc23)c1] | 296 |
| 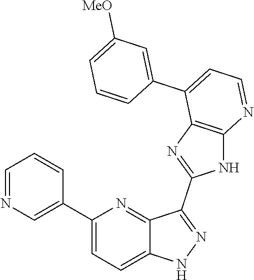 [SMILES: COc1cccc(-c2ccnc3[nH]c(-c4n[nH]c5ccc(-c6cccnc6)nc45)nc23)c1] | 297 |
| 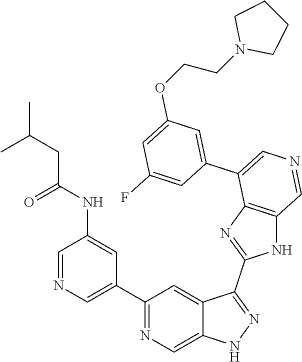 [SMILES: CC(C)CC(=O)Nc1cncc(-c2cc3c(-c4nc5c(-c6cc(F)cc(OCCN7CCCC7)c6)cncc5[nH]4)n[nH]c3cn2)c1] | 298 |
| 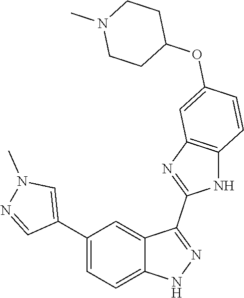 [SMILES: CN1CCC(Oc2ccc3[nH]c(-c4n[nH]c5ccc(-c6cnn(C)c6)cc45)nc3c2)CC1] | 299 |
| 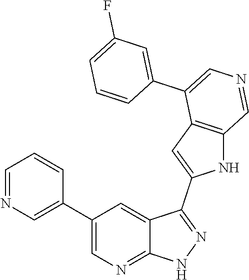 [SMILES: Fc1cccc(-c2cncc3[nH]c(-c4n[nH]c5ncc(-c6cccnc6)cc45)cc23)c1] | 300 |
| 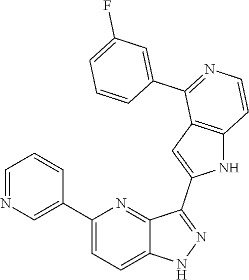 [SMILES: Fc1cccc(-c2nccc3[nH]c(-c4n[nH]c5ccc(-c6cccnc6)nc45)cc23)c1] | 301 |
| 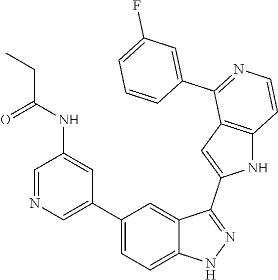 [SMILES: CCC(=O)Nc1cncc(-c2ccc3[nH]nc(-c4cc5c(-c6cccc(F)c6)nccc5[nH]4)c3c2)c1] | 302 |
| 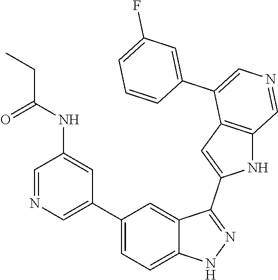 [SMILES: CCC(=O)Nc1cncc(-c2ccc3[nH]nc(-c4cc5c(-c6cccc(F)c6)cncc5[nH]4)c3c2)c1] | 303 |
| 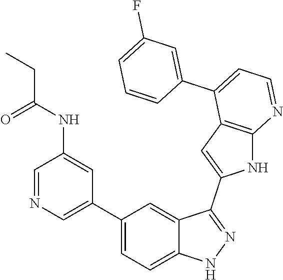 [SMILES: CCC(=O)Nc1cncc(-c2ccc3[nH]nc(-c4cc5c(-c6cccc(F)c6)ccnc5[nH]4)c3c2)c1] | 304 |
| 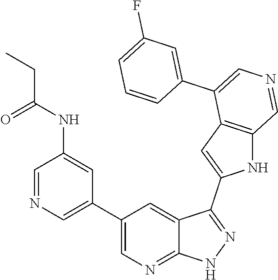 [SMILES: CCC(=O)Nc1cncc(-c2cnc3[nH]nc(-c4cc5c(-c6cccc(F)c6)cncc5[nH]4)c3c2)c1] | 305 |
| 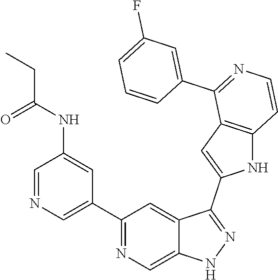 [SMILES: CCC(=O)Nc1cncc(-c2cc3c(-c4cc5c(-c6cccc(F)c6)nccc5[nH]4)n[nH]c3cn2)c1] | 306 |
| 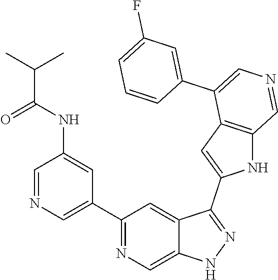 [SMILES: CC(C)C(=O)Nc1cncc(-c2cc3c(-c4cc5c(-c6cccc(F)c6)cncc5[nH]4)n[nH]c3cn2)c1] | 307 |
| 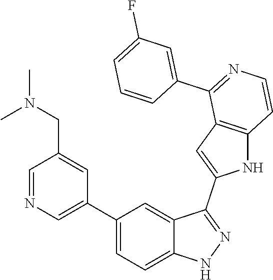 [SMILES: CN(C)Cc1cncc(-c2ccc3[nH]nc(-c4cc5c(-c6cccc(F)c6)nccc5[nH]4)c3c2)c1] | 308 |
| 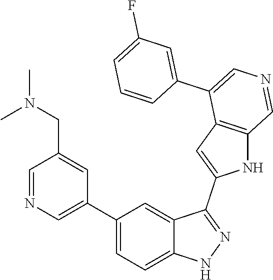 [SMILES: CN(C)Cc1cncc(-c2ccc3[nH]nc(-c4cc5c(-c6cccc(F)c6)cncc5[nH]4)c3c2)c1] | 309 |
| 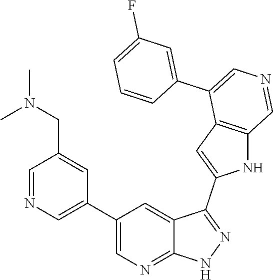 [SMILES: CN(C)Cc1cncc(-c2cnc3[nH]nc(-c4cc5c(-c6cccc(F)c6)cncc5[nH]4)c3c2)c1] | 310 |
| 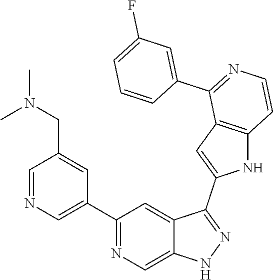 [SMILES: CN(C)Cc1cncc(-c2cc3c(-c4cc5c(-c6cccc(F)c6)nccc5[nH]4)n[nH]c3cn2)c1] | 311 |
| 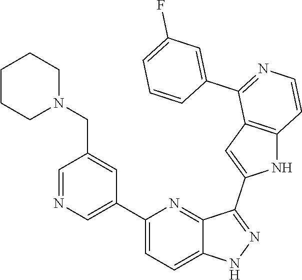 [SMILES: Fc1cccc(-c2nccc3[nH]c(-c4n[nH]c5ccc(-c6cncc(CN7CCCCC7)c6)nc45)cc23)c1] | 312 |
| 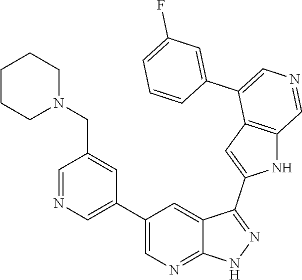 [SMILES: Fc1cccc(-c2cncc3[nH]c(-c4n[nH]c5ncc(-c6cncc(CN7CCCCC7)c6)cc45)cc23)c1] | 313 |
| 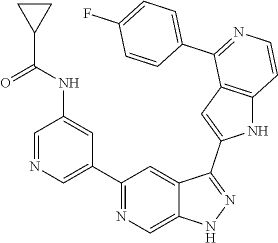 [SMILES: O=C(Nc1cncc(-c2cc3c(-c4cc5c(-c6ccc(F)cc6)nccc5[nH]4)n[nH]c3cn2)c1)C1CC1] | 314 |
| 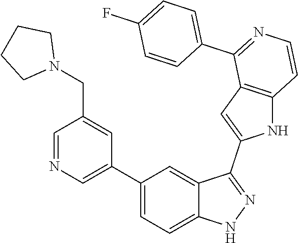 [SMILES: Fc1ccc(-c2nccc3[nH]c(-c4n[nH]c5ccc(-c6cncc(CN7CCCC7)c6)cc45)cc23)cc1] | 315 |
| 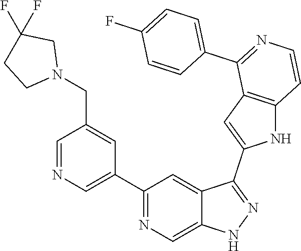 [SMILES: Fc1ccc(-c2nccc3[nH]c(-c4n[nH]c5cnc(-c6cncc(CN7CCC(F)(F)C7)c6)cc45)cc23)cc1] | 316 |
| 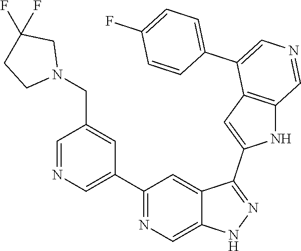 [SMILES: Fc1ccc(-c2cncc3[nH]c(-c4n[nH]c5cnc(-c6cncc(CN7CCC(F)(F)C7)c6)cc45)cc23)cc1] | 317 |
| 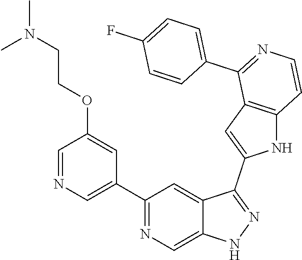 [SMILES: CN(C)CCOc1cncc(-c2cc3c(-c4cc5c(-c6ccc(F)cc6)nccc5[nH]4)n[nH]c3cn2)c1] | 318 |
| 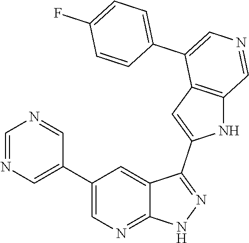 [SMILES: Fc1ccc(-c2cncc3[nH]c(-c4n[nH]c5ncc(-c6cncnc6)cc45)cc23)cc1] | 319 |
| 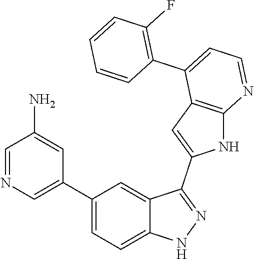 [SMILES: Nc1cncc(-c2ccc3[nH]nc(-c4cc5c(-c6ccccc6F)ccnc5[nH]4)c3c2)c1] | 320 |
| 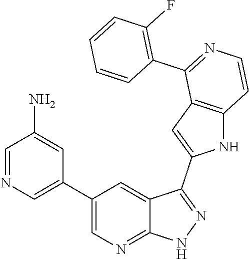 [SMILES: Nc1cncc(-c2cnc3[nH]nc(-c4cc5c(-c6ccccc6F)nccc5[nH]4)c3c2)c1] | 321 |
| 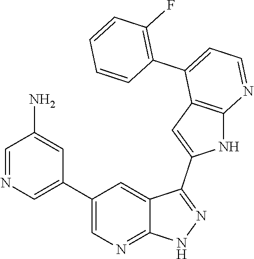 [SMILES: Nc1cncc(-c2cnc3[nH]nc(-c4cc5c(-c6ccccc6F)ccnc5[nH]4)c3c2)c1] | 322 |
| 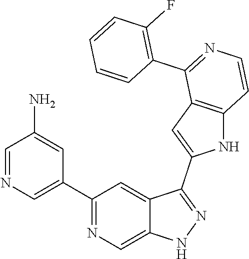 [SMILES: Nc1cncc(-c2cc3c(-c4cc5c(-c6ccccc6F)nccc5[nH]4)n[nH]c3cn2)c1] | 323 |
| 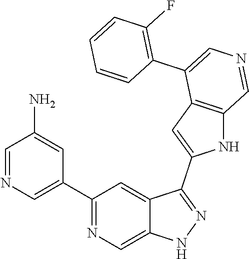 [SMILES: Nc1cncc(-c2cc3c(-c4cc5c(-c6ccccc6F)cncc5[nH]4)n[nH]c3cn2)c1] | 324 |
| 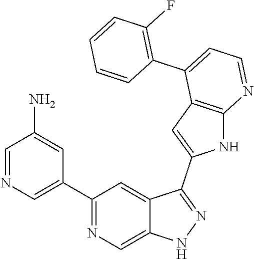 [SMILES: Nc1cncc(-c2cc3c(-c4cc5c(-c6ccccc6F)ccnc5[nH]4)n[nH]c3cn2)c1] | 325 |
| 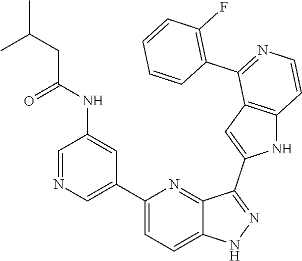 [SMILES: CC(C)CC(=O)Nc1cncc(-c2ccc3[nH]nc(-c4cc5c(-c6ccccc6F)nccc5[nH]4)c3n2)c1] | 326 |
| 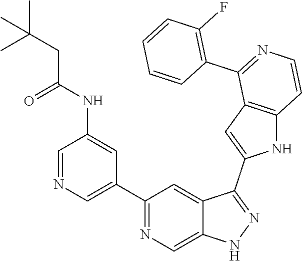 [SMILES: CC(C)(C)CC(=O)Nc1cncc(-c2cc3c(-c4cc5c(-c6ccccc6F)nccc5[nH]4)n[nH]c3cn2)c1] | 327 |
| 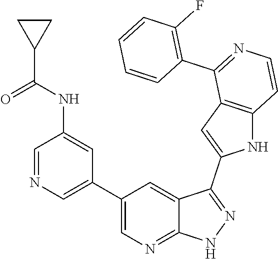 [SMILES: O=C(Nc1cncc(-c2cnc3[nH]nc(-c4cc5c(-c6ccccc6F)nccc5[nH]4)c3c2)c1)C1CC1] | 328 |
| 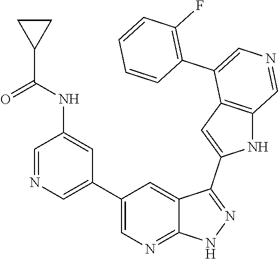 [SMILES: O=C(Nc1cncc(-c2cnc3[nH]nc(-c4cc5c(-c6ccccc6F)cncc5[nH]4)c3c2)c1)C1CC1] | 329 |
| 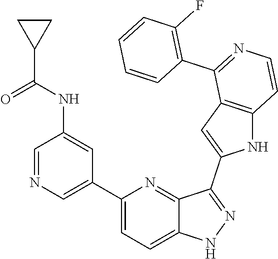 [SMILES: O=C(Nc1cncc(-c2ccc3[nH]nc(-c4cc5c(-c6ccccc6F)nccc5[nH]4)c3n2)c1)C1CC1] | 330 |
| 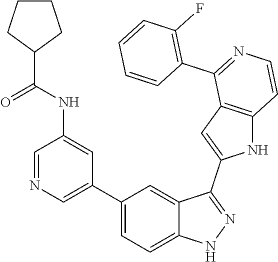 [SMILES: O=C(Nc1cncc(-c2ccc3[nH]nc(-c4cc5c(-c6ccccc6F)nccc5[nH]4)c3c2)c1)C1CCCC1] | 331 |
| 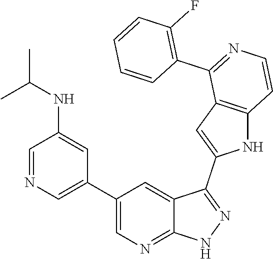 [SMILES: CC(C)Nc1cncc(-c2cnc3[nH]nc(-c4cc5c(-c6ccccc6F)nccc5[nH]4)c3c2)c1] | 332 |
| 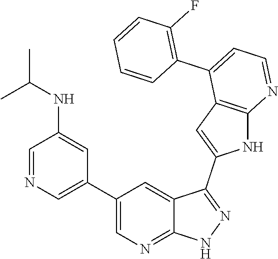 [SMILES: CC(C)Nc1cncc(-c2cnc3[nH]nc(-c4cc5c(-c6ccccc6F)ccnc5[nH]4)c3c2)c1] | 333 |
| 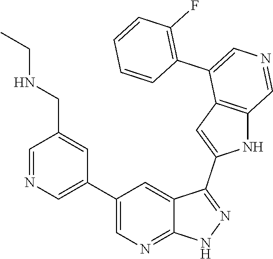 [SMILES: CCNCc1cncc(-c2cnc3[nH]nc(-c4cc5c(-c6ccccc6F)cncc5[nH]4)c3c2)c1] | 334 |
| 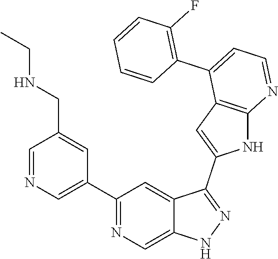 [SMILES: CCNCc1cncc(-c2cc3c(-c4cc5c(-c6ccccc6F)ccnc5[nH]4)n[nH]c3cn2)c1] | 335 |
| 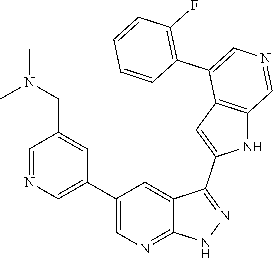 [SMILES: CN(C)Cc1cncc(-c2cnc3[nH]nc(-c4cc5c(-c6ccccc6F)cncc5[nH]4)c3c2)c1] | 336 |
| 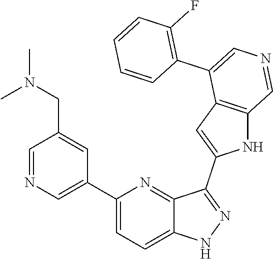 [SMILES: CN(C)Cc1cncc(-c2ccc3[nH]nc(-c4cc5c(-c6ccccc6F)cncc5[nH]4)c3n2)c1] | 337 |
| 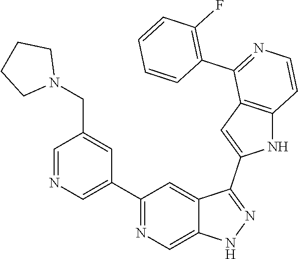 [SMILES: Fc1ccccc1-c1nccc2[nH]c(-c3n[nH]c4cnc(-c5cncc(CN6CCCC6)c5)cc34)cc12] | 338 |
| 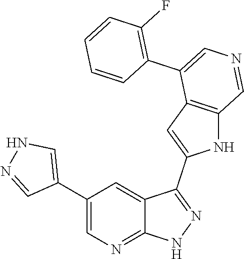 [SMILES: Fc1ccccc1-c1cncc2[nH]c(-c3n[nH]c4ncc(-c5cn[nH]c5)cc34)cc12] | 339 |
| 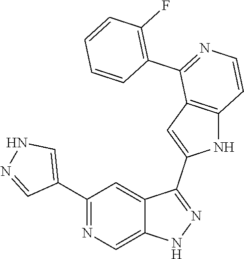 [SMILES: Fc1ccccc1-c1nccc2[nH]c(-c3n[nH]c4cnc(-c5cn[nH]c5)cc34)cc12] | 340 |
| 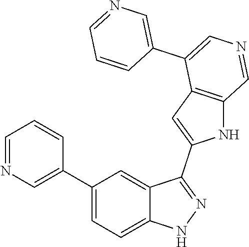 [SMILES: c1cncc(-c2ccc3[nH]nc(-c4cc5c(-c6cccnc6)cncc5[nH]4)c3c2)c1] | 341 |
| 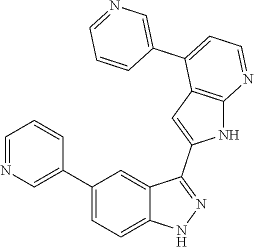 [SMILES: c1cncc(-c2ccc3[nH]nc(-c4cc5c(-c6cccnc6)ccnc5[nH]4)c3c2)c1] | 342 |
| 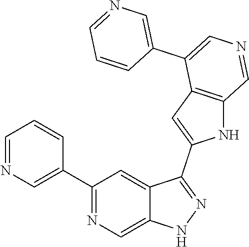 [SMILES: c1cncc(-c2cc3c(-c4cc5c(-c6cccnc6)cncc5[nH]4)n[nH]c3cn2)c1] | 343 |
| 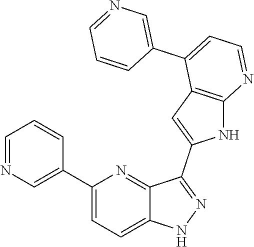 [SMILES: c1cncc(-c2ccc3[nH]nc(-c4cc5c(-c6cccnc6)ccnc5[nH]4)c3n2)c1] | 344 |
| 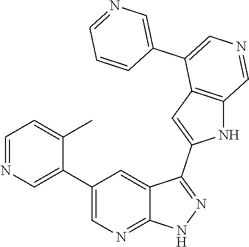 [SMILES: Cc1ccncc1-c1cnc2[nH]nc(-c3cc4c(-c5cccnc5)cncc4[nH]3)c2c1] | 345 |
| 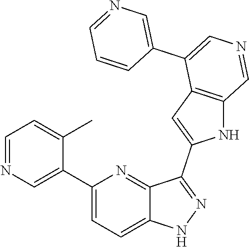 [SMILES: Cc1ccncc1-c1ccc2[nH]nc(-c3cc4c(-c5cccnc5)cncc4[nH]3)c2n1] | 346 |
| 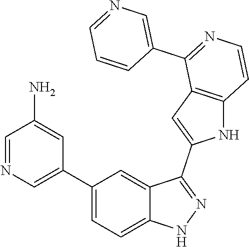 [SMILES: Nc1cncc(-c2ccc3[nH]nc(-c4cc5c(-c6cccnc6)nccc5[nH]4)c3c2)c1] | 347 |
| 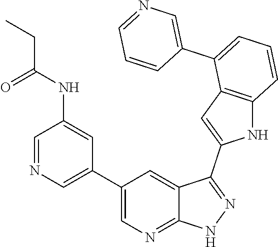 [SMILES: CCC(=O)Nc1cncc(-c2cnc3[nH]nc(-c4cc5c(-c6cccnc6)cccc5[nH]4)c3c2)c1] | 348 |
| 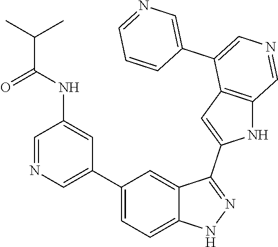 [SMILES: CC(C)C(=O)Nc1cncc(-c2ccc3[nH]nc(-c4cc5c(-c6cccnc6)cncc5[nH]4)c3c2)c1] | 349 |
| 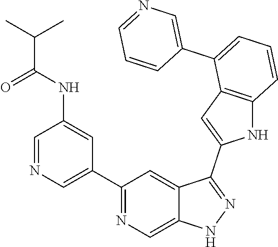 [SMILES: CC(C)C(=O)Nc1cncc(-c2cc3c(-c4cc5c(-c6cccnc6)cccc5[nH]4)n[nH]c3cn2)c1] | 350 |
| 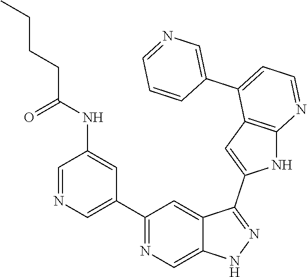 [SMILES: CCCCC(=O)Nc1cncc(-c2cc3c(-c4cc5c(-c6cccnc6)ccnc5[nH]4)n[nH]c3cn2)c1] | 351 |
| 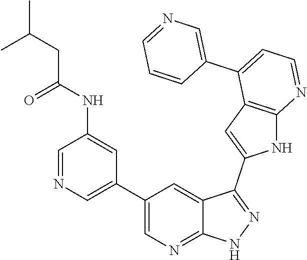 [SMILES: CC(C)CC(=O)Nc1cncc(-c2cnc3[nH]nc(-c4cc5c(-c6cccnc6)ccnc5[nH]4)c3c2)c1] | 352 |
| 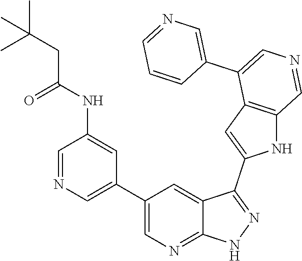 [SMILES: CC(C)(C)CC(=O)Nc1cncc(-c2cnc3[nH]nc(-c4cc5c(-c6cccnc6)cncc5[nH]4)c3c2)c1] | 353 |
| 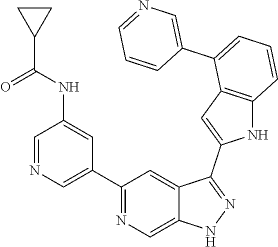 [SMILES: O=C(Nc1cncc(-c2cc3c(-c4cc5c(-c6cccnc6)cccc5[nH]4)n[nH]c3cn2)c1)C1CC1] | 354 |
| 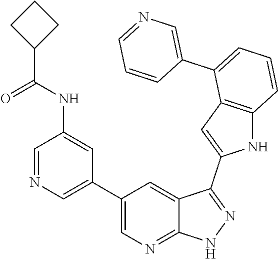 [SMILES: O=C(Nc1cncc(-c2cnc3[nH]nc(-c4cc5c(-c6cccnc6)cccc5[nH]4)c3c2)c1)C1CCC1] | 355 |
| 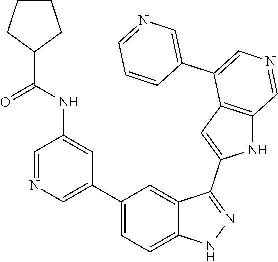 [SMILES: O=C(Nc1cncc(-c2ccc3[nH]nc(-c4cc5c(-c6cccnc6)cncc5[nH]4)c3c2)c1)C1CCCC1] | 356 |
| 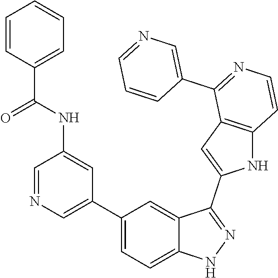 [SMILES: O=C(Nc1cncc(-c2ccc3[nH]nc(-c4cc5c(-c6cccnc6)nccc5[nH]4)c3c2)c1)c1ccccc1] | 357 |
| 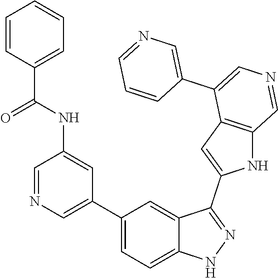 [SMILES: O=C(Nc1cncc(-c2ccc3[nH]nc(-c4cc5c(-c6cccnc6)cncc5[nH]4)c3c2)c1)c1ccccc1] | 358 |
| 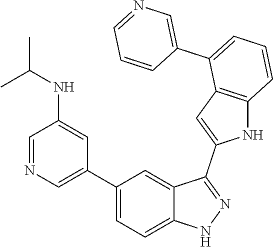 [SMILES: CC(C)Nc1cncc(-c2ccc3[nH]nc(-c4cc5c(-c6cccnc6)cccc5[nH]4)c3c2)c1] | 359 |
| 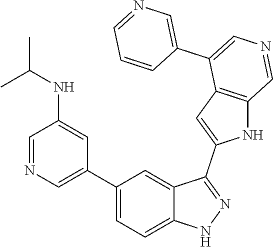 [SMILES: CC(C)Nc1cncc(-c2ccc3[nH]nc(-c4cc5c(-c6cccnc6)cncc5[nH]4)c3c2)c1] | 360 |
| 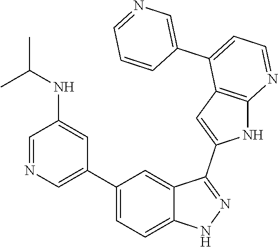 [SMILES: CC(C)Nc1cncc(-c2ccc3[nH]nc(-c4cc5c(-c6cccnc6)ccnc5[nH]4)c3c2)c1] | 361 |
| 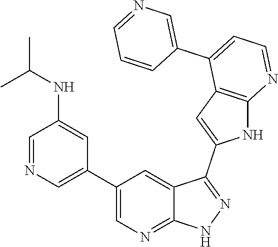 [SMILES: CC(C)Nc1cncc(-c2cnc3[nH]nc(-c4cc5c(-c6cccnc6)ccnc5[nH]4)c3c2)c1] | 362 |
| 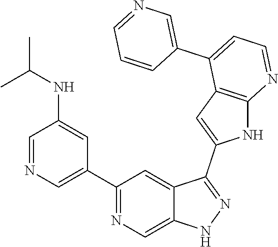 [SMILES: CC(C)Nc1cncc(-c2cc3c(-c4cc5c(-c6cccnc6)ccnc5[nH]4)n[nH]c3cn2)c1] | 363 |
| 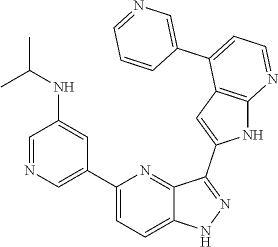 [SMILES: CC(C)Nc1cncc(-c2ccc3[nH]nc(-c4cc5c(-c6cccnc6)ccnc5[nH]4)c3n2)c1] | 364 |
| 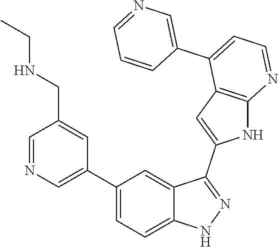 [SMILES: CCNCc1cncc(-c2ccc3[nH]nc(-c4cc5c(-c6cccnc6)ccnc5[nH]4)c3c2)c1] | 365 |
| 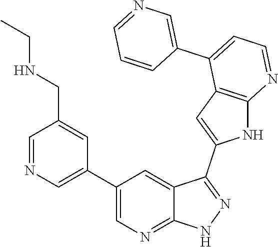 [SMILES: CCNCc1cncc(-c2cnc3[nH]nc(-c4cc5c(-c6cccnc6)ccnc5[nH]4)c3c2)c1] | 366 |
| 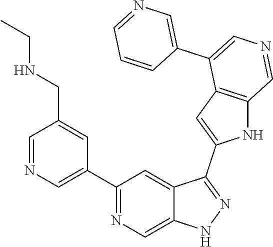 [SMILES: CCNCc1cncc(-c2cc3c(-c4cc5c(-c6cccnc6)cncc5[nH]4)n[nH]c3cn2)c1] | 367 |
| 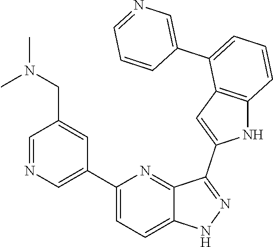 [SMILES: CN(C)Cc1cncc(-c2ccc3[nH]nc(-c4cc5c(-c6cccnc6)cccc5[nH]4)c3n2)c1] | 368 |
| 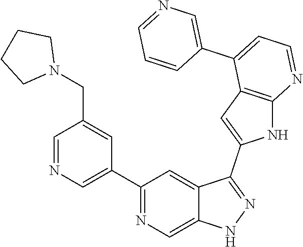 [SMILES: c1cncc(-c2ccnc3[nH]c(-c4n[nH]c5cnc(-c6cncc(CN7CCCC7)c6)cc45)cc23)c1] | 369 |
| 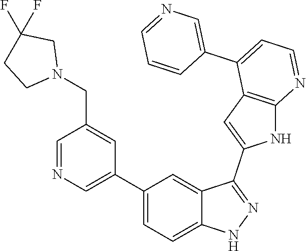 [SMILES: FC1(F)CCN(Cc2cncc(-c3ccc4[nH]nc(-c5cc6c(-c7cccnc7)ccnc6[nH]5)c4c3)c2)C1] | 370 |
| 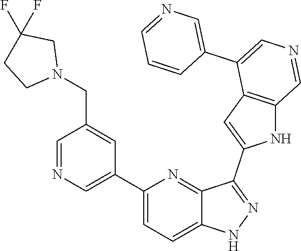 [SMILES: FC1(F)CCN(Cc2cncc(-c3ccc4[nH]nc(-c5cc6c(-c7cccnc7)cncc6[nH]5)c4n3)c2)C1] | 371 |
| 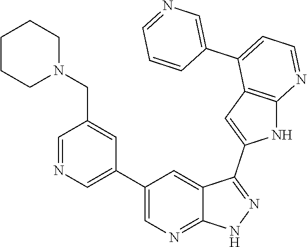 [SMILES: c1cncc(-c2ccnc3[nH]c(-c4n[nH]c5ncc(-c6cncc(CN7CCCCC7)c6)cc45)cc23)c1] | 372 |
| 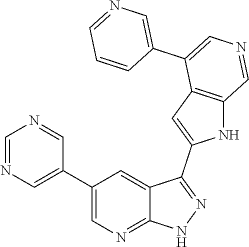 [SMILES: c1cncc(-c2cncc3[nH]c(-c4n[nH]c5ncc(-c6cncnc6)cc45)cc23)c1] | 373 |
| 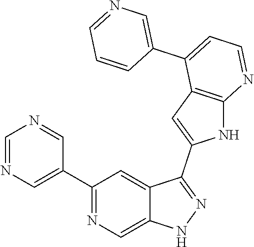 [SMILES: c1cncc(-c2ccnc3[nH]c(-c4n[nH]c5cnc(-c6cncnc6)cc45)cc23)c1] | 374 |
| 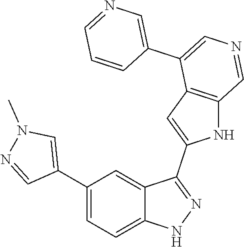 [SMILES: Cn1cc(-c2ccc3[nH]nc(-c4cc5c(-c6cccnc6)cncc5[nH]4)c3c2)cn1] | 375 |
| 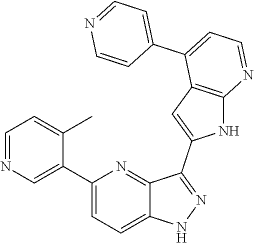 [SMILES: Cc1ccncc1-c1ccc2[nH]nc(-c3cc4c(-c5ccncc5)ccnc4[nH]3)c2n1] | 376 |
| 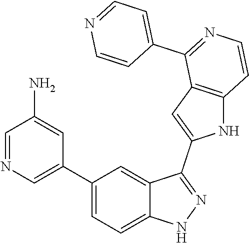 [SMILES: Nc1cncc(-c2ccc3[nH]nc(-c4cc5c(-c6ccncc6)nccc5[nH]4)c3c2)c1] | 377 |
| 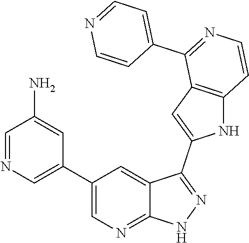 [SMILES: Nc1cncc(-c2cnc3[nH]nc(-c4cc5c(-c6ccncc6)nccc5[nH]4)c3c2)c1] | 378 |
| 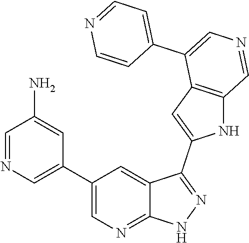 [SMILES: Nc1cncc(-c2cnc3[nH]nc(-c4cc5c(-c6ccncc6)cncc5[nH]4)c3c2)c1] | 379 |
| 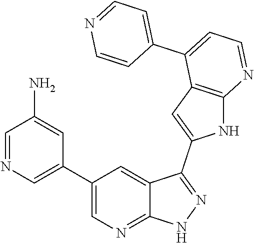 [SMILES: Nc1cncc(-c2cnc3[nH]nc(-c4cc5c(-c6ccncc6)ccnc5[nH]4)c3c2)c1] | 380 |
| 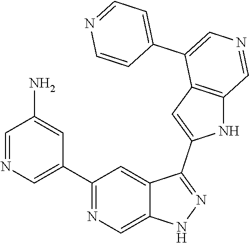 [SMILES: Nc1cncc(-c2cc3c(-c4cc5c(-c6ccncc6)cncc5[nH]4)n[nH]c3cn2)c1] | 381 |
| 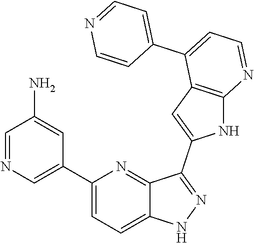 [SMILES: Nc1cncc(-c2ccc3[nH]nc(-c4cc5c(-c6ccncc6)ccnc5[nH]4)c3n2)c1] | 382 |
| 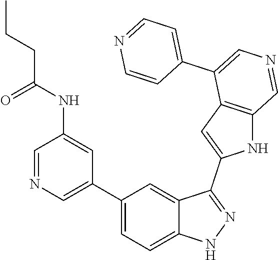 [SMILES: CCCC(=O)Nc1cncc(-c2ccc3[nH]nc(-c4cc5c(-c6ccncc6)cncc5[nH]4)c3c2)c1] | 383 |
| 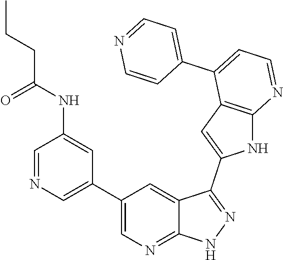 [SMILES: CCCC(=O)Nc1cncc(-c2cnc3[nH]nc(-c4cc5c(-c6ccncc6)ccnc5[nH]4)c3c2)c1] | 384 |
| 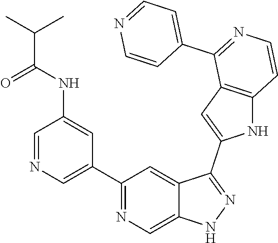 [SMILES: CC(C)C(=O)Nc1cncc(-c2cc3c(-c4cc5c(-c6ccncc6)nccc5[nH]4)n[nH]c3cn2)c1] | 385 |
| 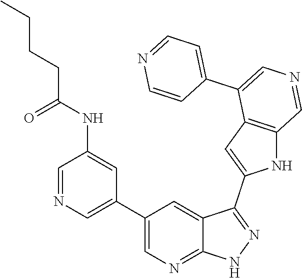 [SMILES: CCCCC(=O)Nc1cncc(-c2cnc3[nH]nc(-c4cc5c(-c6ccncc6)cncc5[nH]4)c3c2)c1] | 386 |
| 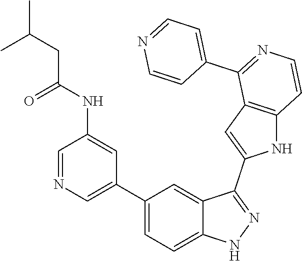 [SMILES: CC(C)CC(=O)Nc1cncc(-c2ccc3[nH]nc(-c4cc5c(-c6ccncc6)nccc5[nH]4)c3c2)c1] | 387 |
| 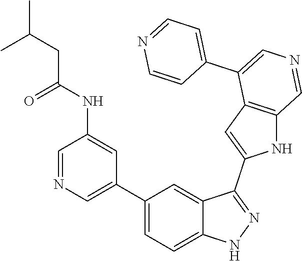 [SMILES: CC(C)CC(=O)Nc1cncc(-c2ccc3[nH]nc(-c4cc5c(-c6ccncc6)cncc5[nH]4)c3c2)c1] | 388 |
| 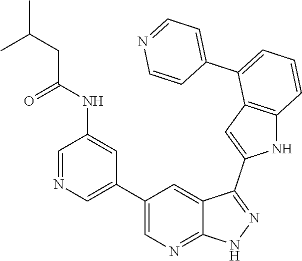 [SMILES: CC(C)CC(=O)Nc1cncc(-c2cnc3[nH]nc(-c4cc5c(-c6ccncc6)cccc5[nH]4)c3c2)c1] | 389 |
| 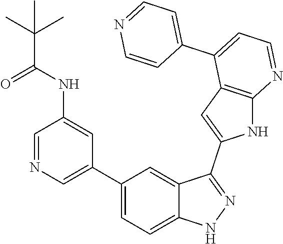 [SMILES: CC(C)(C)C(=O)Nc1cncc(-c2ccc3[nH]nc(-c4cc5c(-c6ccncc6)ccnc5[nH]4)c3c2)c1] | 390 |
| 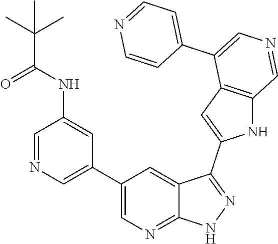 [SMILES: CC(C)(C)C(=O)Nc1cncc(-c2cnc3[nH]nc(-c4cc5c(-c6ccncc6)cncc5[nH]4)c3c2)c1] | 391 |
| 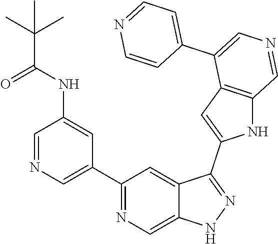 [SMILES: CC(C)(C)C(=O)Nc1cncc(-c2cc3c(-c4cc5c(-c6ccncc6)cncc5[nH]4)n[nH]c3cn2)c1] | 392 |
| 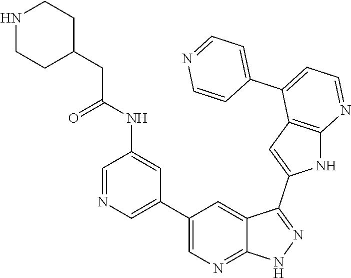 [SMILES: O=C(CC1CCNCC1)Nc1cncc(-c2cnc3[nH]nc(-c4cc5c(-c6ccncc6)ccnc5[nH]4)c3c2)c1] | 393 |
| 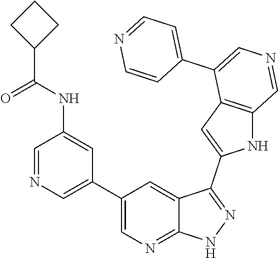 [SMILES: O=C(Nc1cncc(-c2cnc3[nH]nc(-c4cc5c(-c6ccncc6)cncc5[nH]4)c3c2)c1)C1CCC1] | 394 |
| 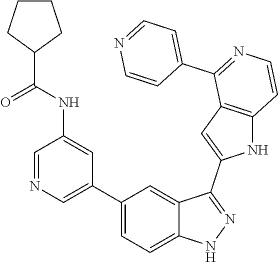 [SMILES: O=C(Nc1cncc(-c2ccc3[nH]nc(-c4cc5c(-c6ccncc6)nccc5[nH]4)c3c2)c1)C1CCCC1] | 395 |
| 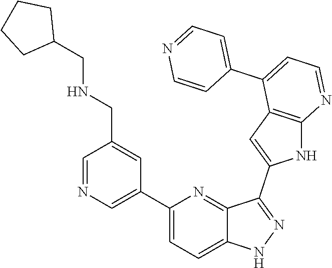 [SMILES: c1cc(-c2ccnc3[nH]c(-c4n[nH]c5ccc(-c6cncc(CNCC7CCCC7)c6)nc45)cc23)ccn1] | 396 |
| 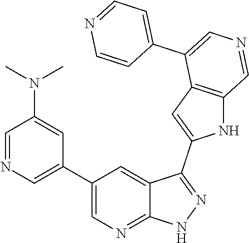 [SMILES: CN(C)c1cncc(-c2cnc3[nH]nc(-c4cc5c(-c6ccncc6)cncc5[nH]4)c3c2)c1] | 397 |
| 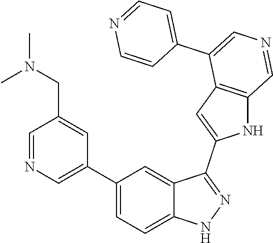 [SMILES: CN(C)Cc1cncc(-c2ccc3[nH]nc(-c4cc5c(-c6ccncc6)cncc5[nH]4)c3c2)c1] | 398 |
| 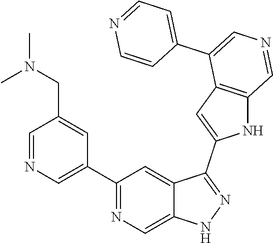 [SMILES: CN(C)Cc1cncc(-c2cc3c(-c4cc5c(-c6ccncc6)cncc5[nH]4)n[nH]c3cn2)c1] | 399 |
| 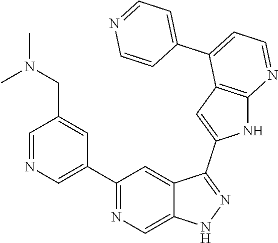 [SMILES: CN(C)Cc1cncc(-c2cc3c(-c4cc5c(-c6ccncc6)ccnc5[nH]4)n[nH]c3cn2)c1] | 400 |
| 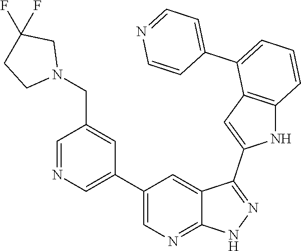 [SMILES: FC1(F)CCN(Cc2cncc(-c3cnc4[nH]nc(-c5cc6c(-c7ccncc7)cccc6[nH]5)c4c3)c2)C1] | 401 |
| 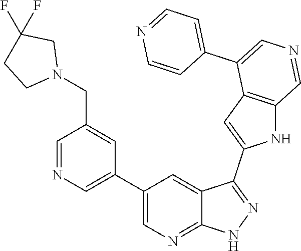 [SMILES: FC1(F)CCN(Cc2cncc(-c3cnc4[nH]nc(-c5cc6c(-c7ccncc7)cncc6[nH]5)c4c3)c2)C1] | 402 |
| 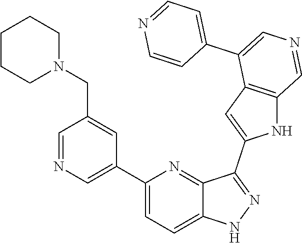 [SMILES: c1cc(-c2cncc3[nH]c(-c4n[nH]c5ccc(-c6cncc(CN7CCCCC7)c6)nc45)cc23)ccn1] | 403 |
| 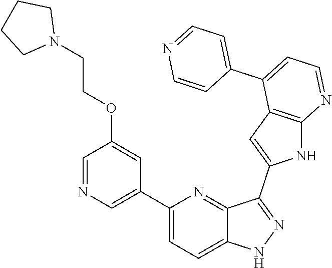 [SMILES: c1cc(-c2ccnc3[nH]c(-c4n[nH]c5ccc(-c6cncc(OCCN7CCCC7)c6)nc45)cc23)ccn1] | 404 |
| 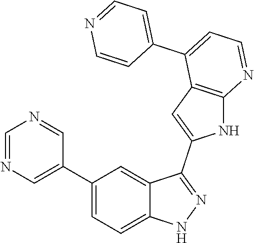 [SMILES: c1cc(-c2ccnc3[nH]c(-c4n[nH]c5ccc(-c6cncnc6)cc45)cc23)ccn1] | 405 |
| 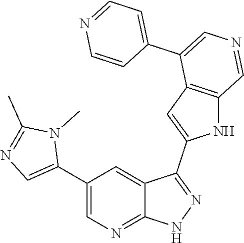 [SMILES: Cc1ncc(-c2cnc3[nH]nc(-c4cc5c(-c6ccncc6)cncc5[nH]4)c3c2)n1C] | 406 |
| 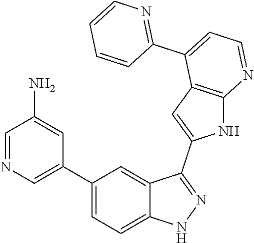 [SMILES: Nc1cncc(-c2ccc3[nH]nc(-c4cc5c(-c6ccccn6)ccnc5[nH]4)c3c2)c1] | 407 |
| 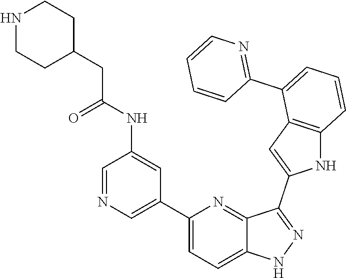 [SMILES: O=C(CC1CCNCC1)Nc1cncc(-c2ccc3[nH]nc(-c4cc5c(-c6ccccn6)cccc5[nH]4)c3n2)c1] | 408 |
| 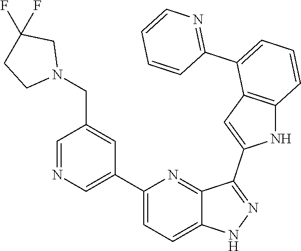 [SMILES: FC1(F)CCN(Cc2cncc(-c3ccc4[nH]nc(-c5cc6c(-c7ccccn7)cccc6[nH]5)c4n3)c2)C1] | 409 |
| 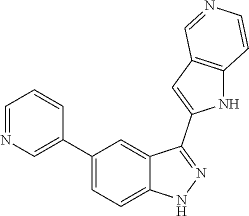 [SMILES: c1cncc(-c2ccc3[nH]nc(-c4cc5cnccc5[nH]4)c3c2)c1] | 410 |
| 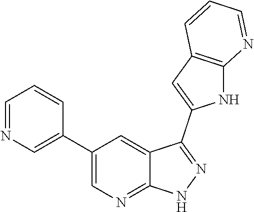 [SMILES: c1cncc(-c2cnc3[nH]nc(-c4cc5cccnc5[nH]4)c3c2)c1] | 411 |
| 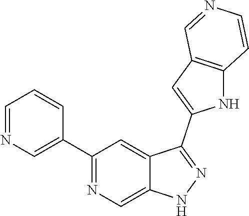 [SMILES: c1cncc(-c2cc3c(-c4cc5cnccc5[nH]4)n[nH]c3cn2)c1] | 412 |
| 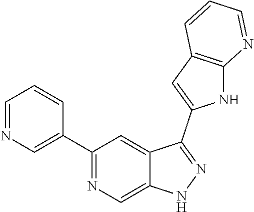 [SMILES: c1cncc(-c2cc3c(-c4cc5cccnc5[nH]4)n[nH]c3cn2)c1] | 413 |
| 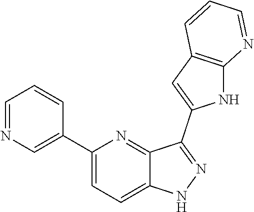 [SMILES: c1cncc(-c2ccc3[nH]nc(-c4cc5cccnc5[nH]4)c3n2)c1] | 414 |
| 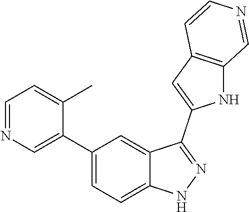 [SMILES: Cc1ccncc1-c1ccc2[nH]nc(-c3cc4ccncc4[nH]3)c2c1] | 415 |
| 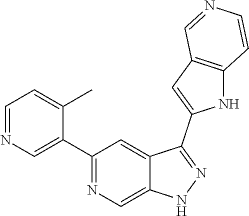 [SMILES: Cc1ccncc1-c1cc2c(-c3cc4cnccc4[nH]3)n[nH]c2cn1] | 416 |
| 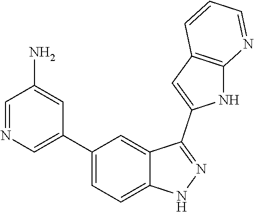 [SMILES: Nc1cncc(-c2ccc3[nH]nc(-c4cc5cccnc5[nH]4)c3c2)c1] | 417 |
| 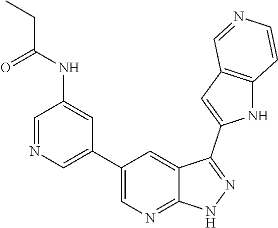 [SMILES: CCC(=O)Nc1cncc(-c2cnc3[nH]nc(-c4cc5cnccc5[nH]4)c3c2)c1] | 418 |
| 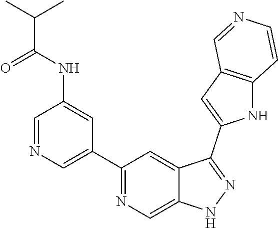 [SMILES: CC(C)C(=O)Nc1cncc(-c2cc3c(-c4cc5cnccc5[nH]4)n[nH]c3cn2)c1] | 419 |
| 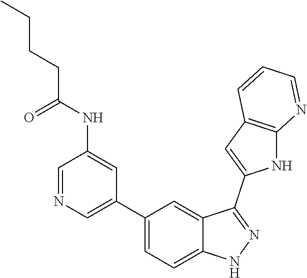 [SMILES: CCCCC(=O)Nc1cncc(-c2ccc3[nH]nc(-c4cc5cccnc5[nH]4)c3c2)c1] | 420 |
| 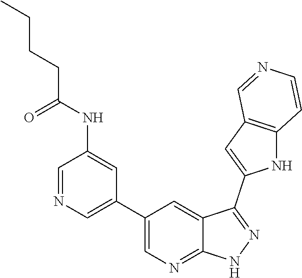 [SMILES: CCCCC(=O)Nc1cncc(-c2cnc3[nH]nc(-c4cc5cnccc5[nH]4)c3c2)c1] | 421 |
| 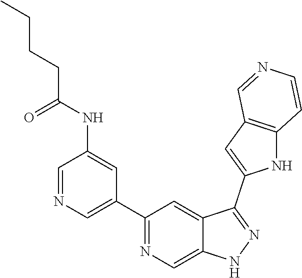 [SMILES: CCCCC(=O)Nc1cncc(-c2cc3c(-c4cc5cnccc5[nH]4)n[nH]c3cn2)c1] | 422 |
| 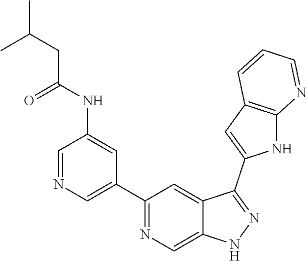 [SMILES: CC(C)CC(=O)Nc1cncc(-c2cc3c(-c4cc5cccnc5[nH]4)n[nH]c3cn2)c1] | 423 |
| 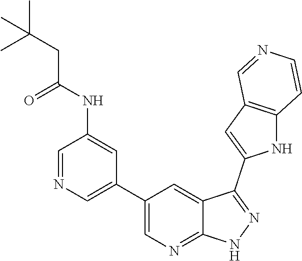 [SMILES: CC(C)(C)CC(=O)Nc1cncc(-c2cnc3[nH]nc(-c4cc5cnccc5[nH]4)c3c2)c1] | 424 |
| 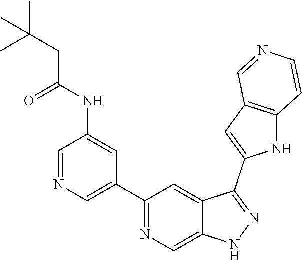 [SMILES: CC(C)(C)CC(=O)Nc1cncc(-c2cc3c(-c4cc5cnccc5[nH]4)n[nH]c3cn2)c1] | 425 |
| 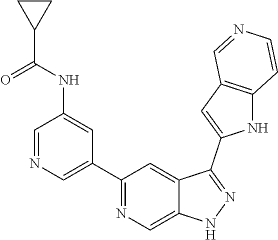 [SMILES: O=C(Nc1cncc(-c2cc3c(-c4cc5cnccc5[nH]4)n[nH]c3cn2)c1)C1CC1] | 426 |
| 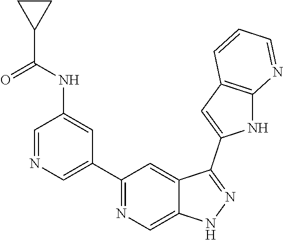 [SMILES: O=C(Nc1cncc(-c2cc3c(-c4cc5cccnc5[nH]4)n[nH]c3cn2)c1)C1CC1] | 427 |
| 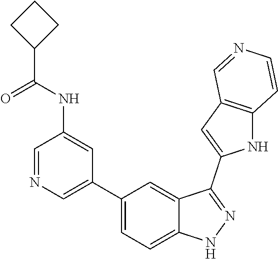 [SMILES: O=C(Nc1cncc(-c2ccc3[nH]nc(-c4cc5cnccc5[nH]4)c3c2)c1)C1CCC1] | 428 |
| 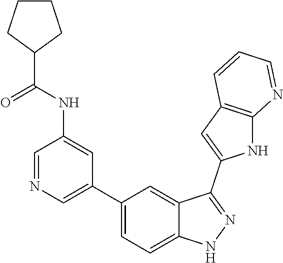 [SMILES: O=C(Nc1cncc(-c2ccc3[nH]nc(-c4cc5cccnc5[nH]4)c3c2)c1)C1CCCC1] | 429 |
| 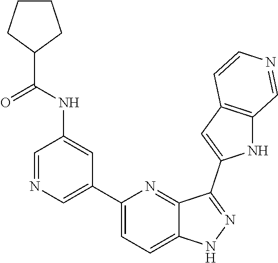 [SMILES: O=C(Nc1cncc(-c2ccc3[nH]nc(-c4cc5ccncc5[nH]4)c3n2)c1)C1CCCC1] | 430 |
| 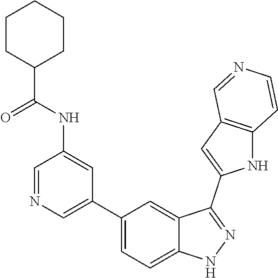 [SMILES: O=C(Nc1cncc(-c2ccc3[nH]nc(-c4cc5cnccc5[nH]4)c3c2)c1)C1CCCCC1] | 431 |
| 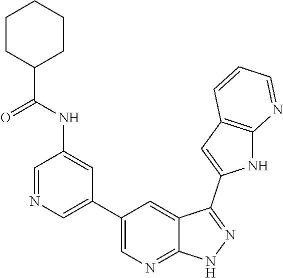 [SMILES: O=C(Nc1cncc(-c2cnc3[nH]nc(-c4cc5cccnc5[nH]4)c3c2)c1)C1CCCCC1] | 432 |
| 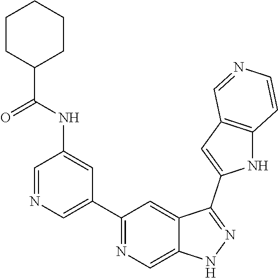 [SMILES: O=C(Nc1cncc(-c2cc3c(-c4cc5cnccc5[nH]4)n[nH]c3cn2)c1)C1CCCCC1] | 433 |
| 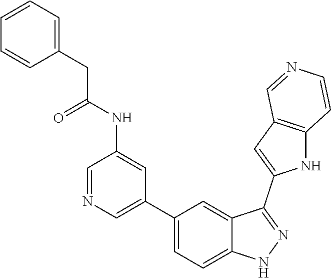 [SMILES: O=C(Cc1ccccc1)Nc1cncc(-c2ccc3[nH]nc(-c4cc5cnccc5[nH]4)c3c2)c1] | 434 |
| 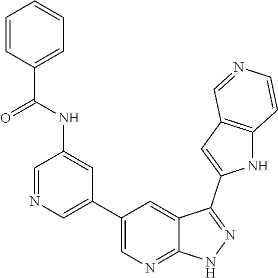 [SMILES: O=C(Nc1cncc(-c2cnc3[nH]nc(-c4cc5cnccc5[nH]4)c3c2)c1)c1ccccc1] | 435 |
| 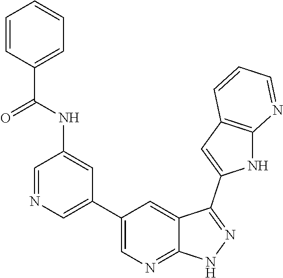 [SMILES: O=C(Nc1cncc(-c2cnc3[nH]nc(-c4cc5cccnc5[nH]4)c3c2)c1)c1ccccc1] | 436 |
| 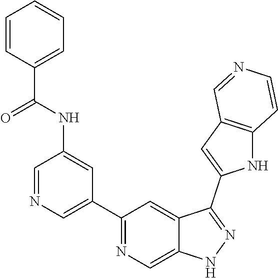 [SMILES: O=C(Nc1cncc(-c2cc3c(-c4cc5cnccc5[nH]4)n[nH]c3cn2)c1)c1ccccc1] | 437 |
| 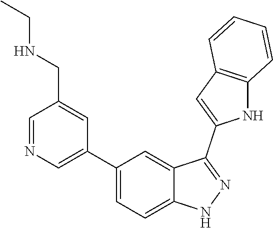 [SMILES: CCNCc1cncc(-c2ccc3[nH]nc(-c4cc5ccccc5[nH]4)c3c2)c1] | 438 |
| 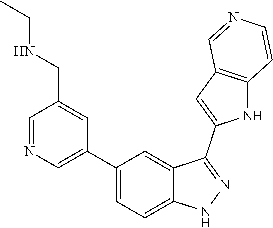 [SMILES: CCNCc1cncc(-c2ccc3[nH]nc(-c4cc5cnccc5[nH]4)c3c2)c1] | 439 |
| 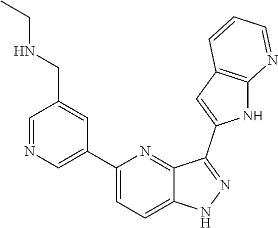 [SMILES: CCNCc1cncc(-c2ccc3[nH]nc(-c4cc5cccnc5[nH]4)c3n2)c1] | 440 |
| 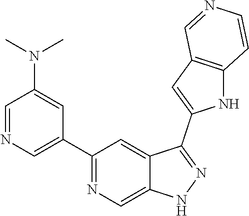 [SMILES: CN(C)c1cncc(-c2cc3c(-c4cc5cnccc5[nH]4)n[nH]c3cn2)c1] | 441 |
| 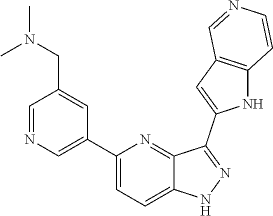 [SMILES: CN(C)Cc1cncc(-c2ccc3[nH]nc(-c4cc5cnccc5[nH]4)c3n2)c1] | 442 |
| 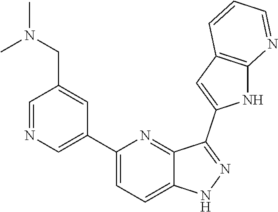 [SMILES: CN(C)Cc1cncc(-c2ccc3[nH]nc(-c4cc5cccnc5[nH]4)c3n2)c1] | 443 |
| 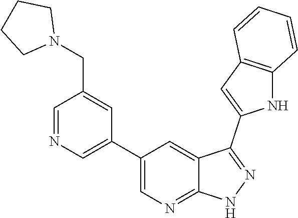 [SMILES: c1ccc2[nH]c(-c3n[nH]c4ncc(-c5cncc(CN6CCCC6)c5)cc34)cc2c1] | 444 |
| 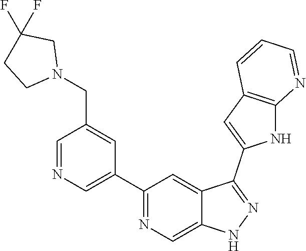 [SMILES: FC1(F)CCN(Cc2cncc(-c3cc4c(-c5cc6cccnc6[nH]5)n[nH]c4cn3)c2)C1] | 445 |
| 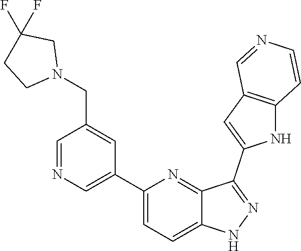 [SMILES: FC1(F)CCN(Cc2cncc(-c3ccc4[nH]nc(-c5cc6cnccc6[nH]5)c4n3)c2)C1] | 446 |
| 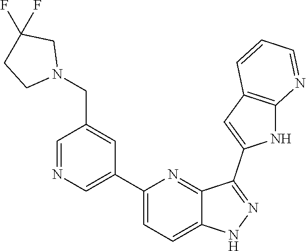 [SMILES: FC1(F)CCN(Cc2cncc(-c3ccc4[nH]nc(-c5cc6cccnc6[nH]5)c4n3)c2)C1] | 447 |
| 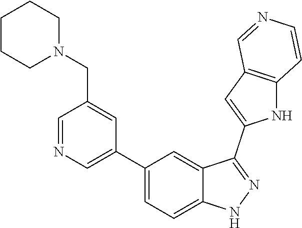 [SMILES: c1cc2[nH]c(-c3n[nH]c4ccc(-c5cncc(CN6CCCCC6)c5)cc34)cc2cn1] | 448 |
| 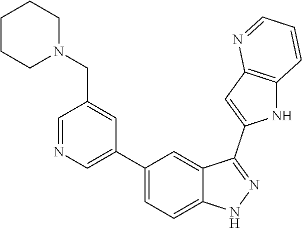 [SMILES: c1cnc2cc(-c3n[nH]c4ccc(-c5cncc(CN6CCCCC6)c5)cc34)[nH]c2c1] | 449 |
| 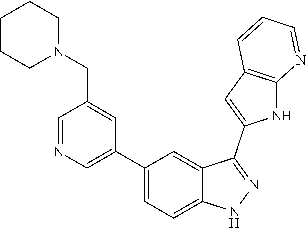 [SMILES: c1cnc2[nH]c(-c3n[nH]c4ccc(-c5cncc(CN6CCCCC6)c5)cc34)cc2c1] | 450 |
| 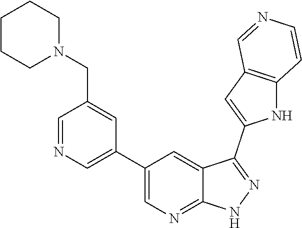 [SMILES: c1cc2[nH]c(-c3n[nH]c4ncc(-c5cncc(CN6CCCCC6)c5)cc34)cc2cn1] | 451 |
| 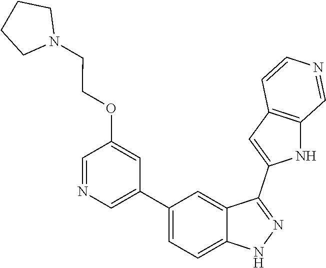 [SMILES: c1cc2cc(-c3n[nH]c4ccc(-c5cncc(OCCN6CCCC6)c5)cc34)[nH]c2cn1] | 452 |
| 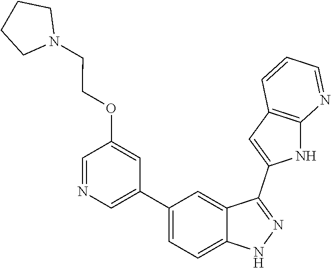 [SMILES: c1cnc2[nH]c(-c3n[nH]c4ccc(-c5cncc(OCCN6CCCC6)c5)cc34)cc2c1] | 453 |
| 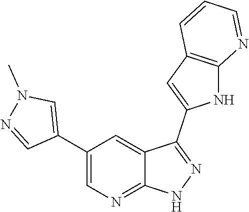 [SMILES: Cn1cc(-c2cnc3[nH]nc(-c4cc5cccnc5[nH]4)c3c2)cn1] | 454 |
| 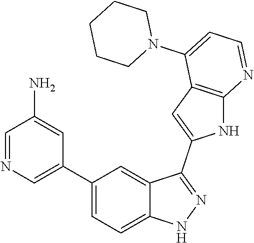 [SMILES: Nc1cncc(-c2ccc3[nH]nc(-c4cc5c(N6CCCCC6)ccnc5[nH]4)c3c2)c1] | 455 |
| 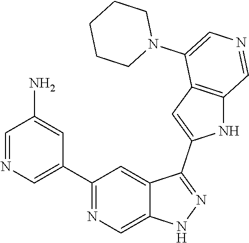 [SMILES: Nc1cncc(-c2cc3c(-c4cc5c(N6CCCCC6)cncc5[nH]4)n[nH]c3cn2)c1] | 456 |
| 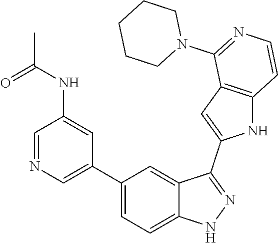 [SMILES: CC(=O)Nc1cncc(-c2ccc3[nH]nc(-c4cc5c(N6CCCCC6)nccc5[nH]4)c3c2)c1] | 457 |
| 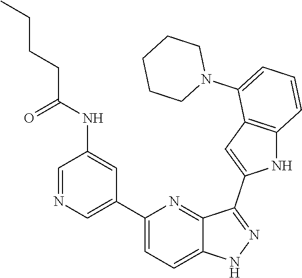 [SMILES: CCCCC(=O)Nc1cncc(-c2ccc3[nH]nc(-c4cc5c(N6CCCCC6)cccc5[nH]4)c3n2)c1] | 458 |
| 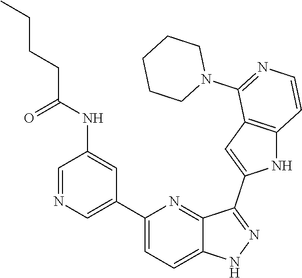 [SMILES: CCCCC(=O)Nc1cncc(-c2ccc3[nH]nc(-c4cc5c(N6CCCCC6)nccc5[nH]4)c3n2)c1] | 459 |
| 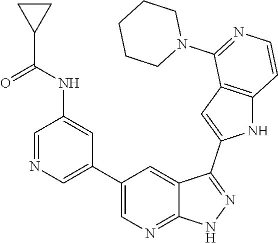 [SMILES: O=C(Nc1cncc(-c2cnc3[nH]nc(-c4cc5c(N6CCCCC6)nccc5[nH]4)c3c2)c1)C1CC1] | 460 |
| 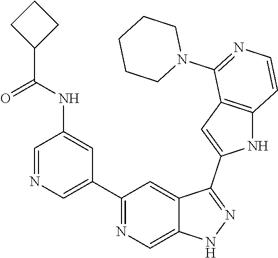 [SMILES: O=C(Nc1cncc(-c2cc3c(-c4cc5c(N6CCCCC6)nccc5[nH]4)n[nH]c3cn2)c1)C1CCC1] | 461 |
| 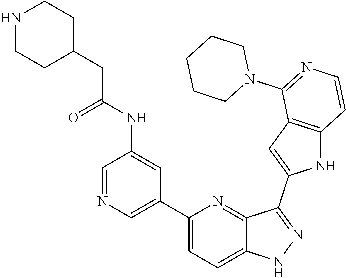 [SMILES: O=C(CC1CCNCC1)Nc1cncc(-c2ccc3[nH]nc(-c4cc5c(N6CCCCC6)nccc5[nH]4)c3n2)c1] | 462 |
| 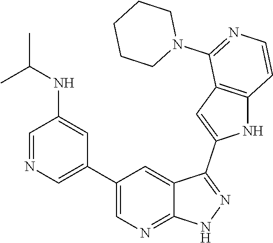 [SMILES: CC(C)Nc1cncc(-c2cnc3[nH]nc(-c4cc5c(N6CCCCC6)nccc5[nH]4)c3c2)c1] | 463 |
| 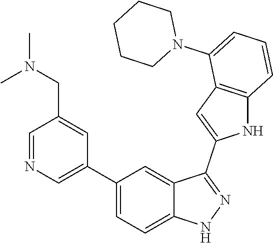 [SMILES: CN(C)Cc1cncc(-c2ccc3[nH]nc(-c4cc5c(N6CCCCC6)cccc5[nH]4)c3c2)c1] | 464 |
| 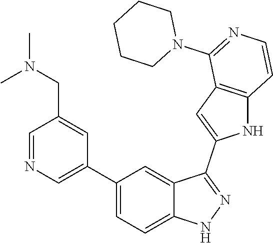 [SMILES: CN(C)Cc1cncc(-c2ccc3[nH]nc(-c4cc5c(N6CCCCC6)nccc5[nH]4)c3c2)c1] | 465 |
| 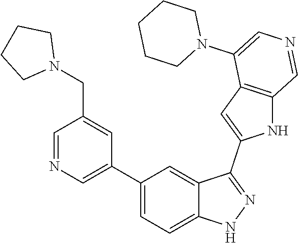 [SMILES: c1ncc(-c2ccc3[nH]nc(-c4cc5c(N6CCCCC6)cncc5[nH]4)c3c2)cc1CN1CCCC1] | 466 |
| 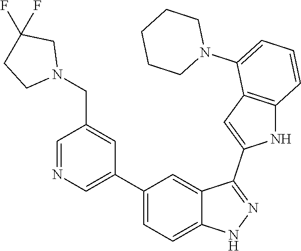 [SMILES: FC1(F)CCN(Cc2cncc(-c3ccc4[nH]nc(-c5cc6c(N7CCCCC7)cccc6[nH]5)c4c3)c2)C1] | 467 |
| 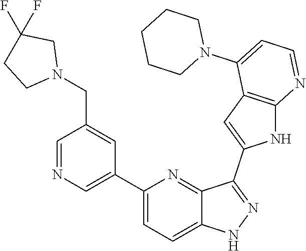 [SMILES: FC1(F)CCN(Cc2cncc(-c3ccc4[nH]nc(-c5cc6c(N7CCCCC7)ccnc6[nH]5)c4n3)c2)C1] | 468 |
| 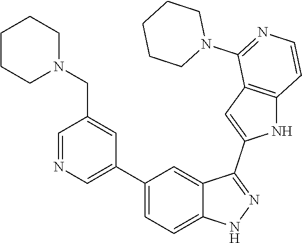 [SMILES: c1cc2[nH]c(-c3n[nH]c4ccc(-c5cncc(CN6CCCCC6)c5)cc34)cc2c(N2CCCCC2)n1] | 469 |
| 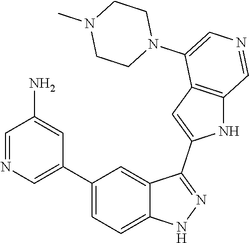 [SMILES: CN1CCN(c2cncc3[nH]c(-c4n[nH]c5ccc(-c6cncc(N)c6)cc45)cc23)CC1] | 470 |
| 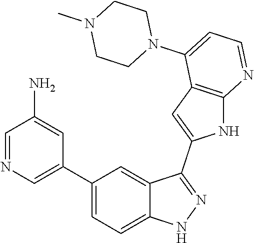 [SMILES: CN1CCN(c2ccnc3[nH]c(-c4n[nH]c5ccc(-c6cncc(N)c6)cc45)cc23)CC1] | 471 |
| 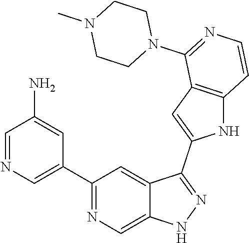 [SMILES: CN1CCN(c2nccc3[nH]c(-c4n[nH]c5cnc(-c6cncc(N)c6)cc45)cc23)CC1] | 472 |
| 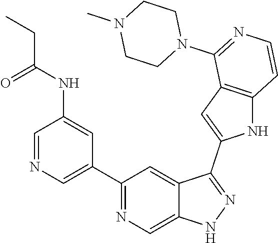 [SMILES: CCC(=O)Nc1cncc(-c2cc3c(-c4cc5c(N6CCN(C)CC6)nccc5[nH]4)n[nH]c3cn2)c1] | 473 |
| 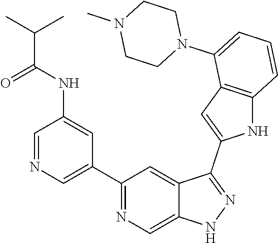 [SMILES: CC(C)C(=O)Nc1cncc(-c2cc3c(-c4cc5c(N6CCN(C)CC6)cccc5[nH]4)n[nH]c3cn2)c1] | 474 |
| 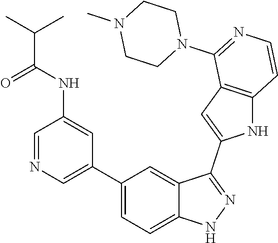 [SMILES: CC(C)C(=O)Nc1cncc(-c2ccc3[nH]nc(-c4cc5c(N6CCN(C)CC6)nccc5[nH]4)c3c2)c1] | 475 |
| 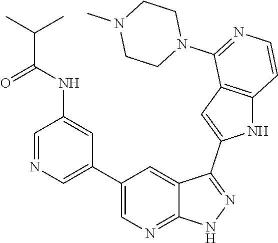 [SMILES: CC(C)C(=O)Nc1cncc(-c2cnc3[nH]nc(-c4cc5c(N6CCN(C)CC6)nccc5[nH]4)c3c2)c1] | 476 |
| 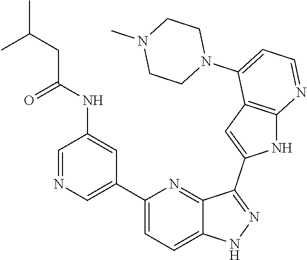 [SMILES: CC(C)CC(=O)Nc1cncc(-c2ccc3[nH]nc(-c4cc5c(N6CCN(C)CC6)ccnc5[nH]4)c3n2)c1] | 477 |
| 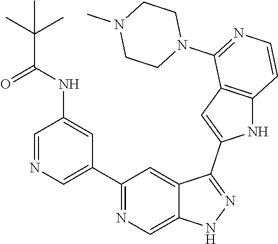 [SMILES: CN1CCN(c2nccc3[nH]c(-c4n[nH]c5cnc(-c6cncc(NC(=O)C(C)(C)C)c6)cc45)cc23)CC1] | 478 |
| 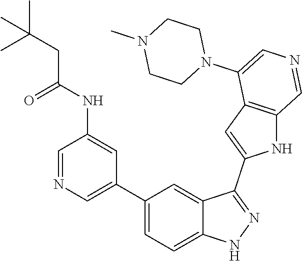 [SMILES: CN1CCN(c2cncc3[nH]c(-c4n[nH]c5ccc(-c6cncc(NC(=O)CC(C)(C)C)c6)cc45)cc23)CC1] | 479 |
| 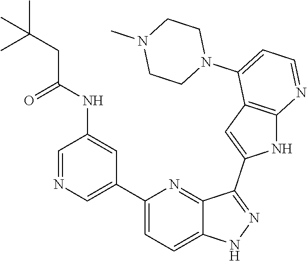 [SMILES: CN1CCN(c2ccnc3[nH]c(-c4n[nH]c5ccc(-c6cncc(NC(=O)CC(C)(C)C)c6)nc45)cc23)CC1] | 480 |
| 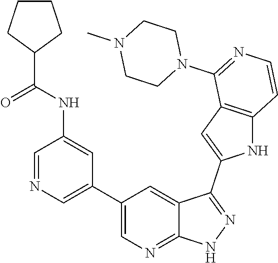 [SMILES: CN1CCN(c2nccc3[nH]c(-c4n[nH]c5ncc(-c6cncc(NC(=O)C7CCCC7)c6)cc45)cc23)CC1] | 481 |
| 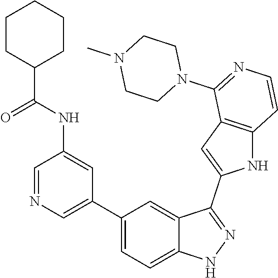 [SMILES: CN1CCN(c2nccc3[nH]c(-c4n[nH]c5ccc(-c6cncc(NC(=O)C7CCCCC7)c6)cc45)cc23)CC1] | 482 |
| 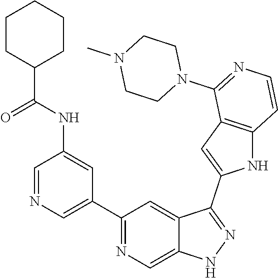 [SMILES: CN1CCN(c2nccc3[nH]c(-c4n[nH]c5cnc(-c6cncc(NC(=O)C7CCCCC7)c6)cc45)cc23)CC1] | 483 |
| 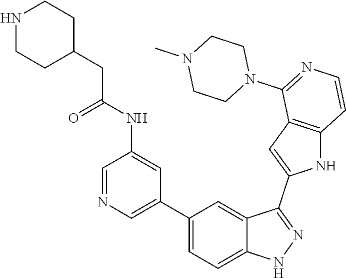 [SMILES: CN1CCN(c2nccc3[nH]c(-c4n[nH]c5ccc(-c6cncc(NC(=O)CC7CCNCC7)c6)cc45)cc23)CC1] | 484 |
| 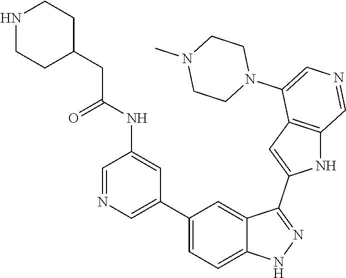 [SMILES: CN1CCN(c2cncc3[nH]c(-c4n[nH]c5ccc(-c6cncc(NC(=O)CC7CCNCC7)c6)cc45)cc23)CC1] | 485 |
| 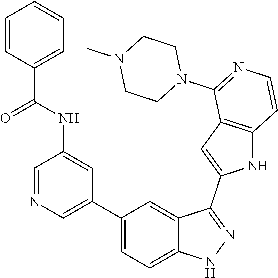 [SMILES: CN1CCN(c2nccc3[nH]c(-c4n[nH]c5ccc(-c6cncc(NC(=O)c7ccccc7)c6)cc45)cc23)CC1] | 486 |
| 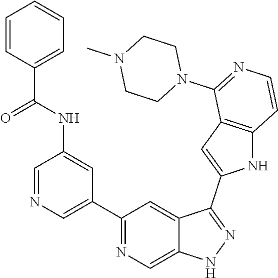 [SMILES: CN1CCN(c2nccc3[nH]c(-c4n[nH]c5cnc(-c6cncc(NC(=O)c7ccccc7)c6)cc45)cc23)CC1] | 487 |
| 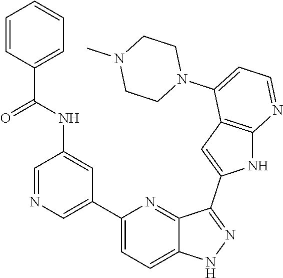 [SMILES: CN1CCN(c2ccnc3[nH]c(-c4n[nH]c5ccc(-c6cncc(NC(=O)c7ccccc7)c6)nc45)cc23)CC1] | 488 |
| 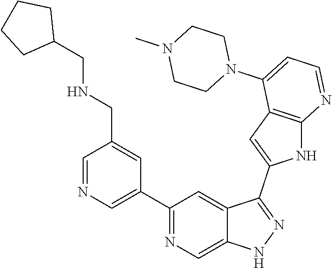 [SMILES: CN1CCN(c2ccnc3[nH]c(-c4n[nH]c5cnc(-c6cncc(CNCC7CCCC7)c6)cc45)cc23)CC1] | 489 |
| 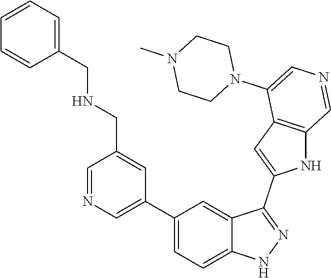 [SMILES: CN1CCN(c2cncc3[nH]c(-c4n[nH]c5ccc(-c6cncc(CNCc7ccccc7)c6)cc45)cc23)CC1] | 490 |
| 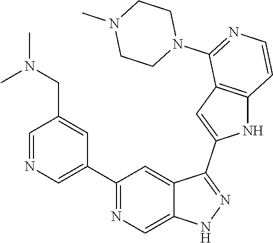 [SMILES: CN(C)Cc1cncc(-c2cc3c(-c4cc5c(N6CCN(C)CC6)nccc5[nH]4)n[nH]c3cn2)c1] | 491 |
| 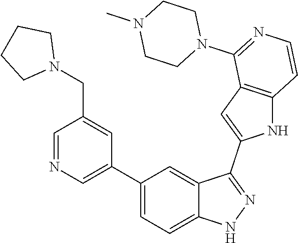 [SMILES: CN1CCN(c2nccc3[nH]c(-c4n[nH]c5ccc(-c6cncc(CN7CCCC7)c6)cc45)cc23)CC1] | 492 |
| 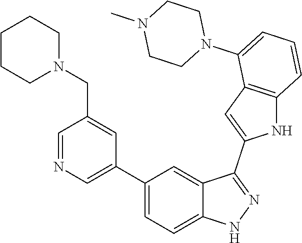 [SMILES: CN1CCN(c2cccc3[nH]c(-c4n[nH]c5ccc(-c6cncc(CN7CCCCC7)c6)cc45)cc23)CC1] | 493 |
| 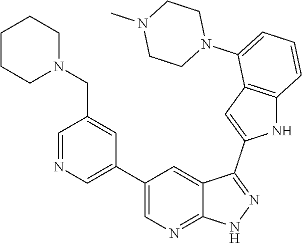 [SMILES: CN1CCN(c2cccc3[nH]c(-c4n[nH]c5ncc(-c6cncc(CN7CCCCC7)c6)cc45)cc23)CC1] | 494 |
| 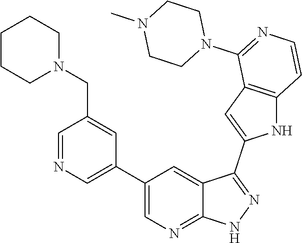 [SMILES: CN1CCN(c2nccc3[nH]c(-c4n[nH]c5ncc(-c6cncc(CN7CCCCC7)c6)cc45)cc23)CC1] | 495 |
| 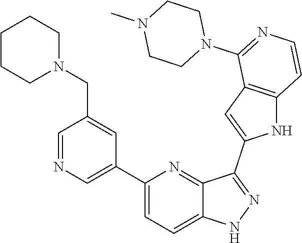 [SMILES: CN1CCN(c2nccc3[nH]c(-c4n[nH]c5ccc(-c6cncc(CN7CCCCC7)c6)nc45)cc23)CC1] | 496 |
| 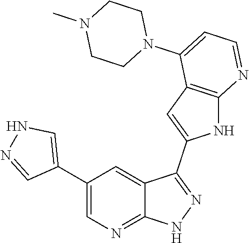 [SMILES: CN1CCN(c2ccnc3[nH]c(-c4n[nH]c5ncc(-c6cn[nH]c6)cc45)cc23)CC1] | 497 |
| 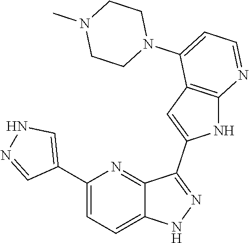 [SMILES: CN1CCN(c2ccnc3[nH]c(-c4n[nH]c5ccc(-c6cn[nH]c6)nc45)cc23)CC1] | 498 |
| 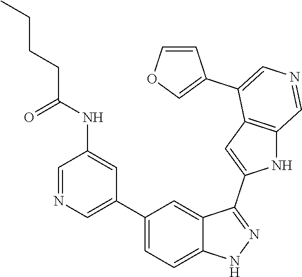 [SMILES: CCCCC(=O)Nc1cncc(-c2ccc3[nH]nc(-c4cc5c(-c6ccoc6)cncc5[nH]4)c3c2)c1] | 499 |
| 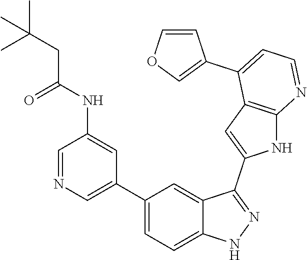 [SMILES: CC(C)(C)CC(=O)Nc1cncc(-c2ccc3[nH]nc(-c4cc5c(-c6ccoc6)ccnc5[nH]4)c3c2)c1] | 500 |
| 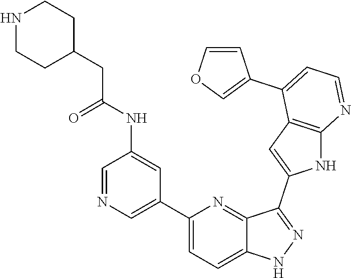 [SMILES: O=C(CC1CCNCC1)Nc1cncc(-c2ccc3[nH]nc(-c4cc5c(-c6ccoc6)ccnc5[nH]4)c3n2)c1] | 501 |
| 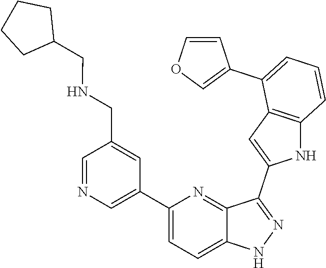 [SMILES: c1cc(-c2ccoc2)c2cc(-c3n[nH]c4ccc(-c5cncc(CNCC6CCCC6)c5)nc34)[nH]c2c1] | 502 |
| 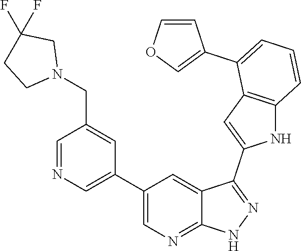 [SMILES: FC1(F)CCN(Cc2cncc(-c3cnc4[nH]nc(-c5cc6c(-c7ccoc7)cccc6[nH]5)c4c3)c2)C1] | 503 |
| 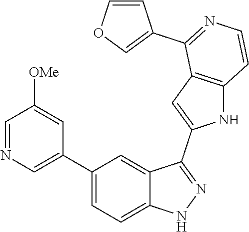 [SMILES: COc1cncc(-c2ccc3[nH]nc(-c4cc5c(-c6ccoc6)nccc5[nH]4)c3c2)c1] | 504 |
| 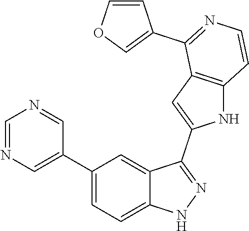 [SMILES: c1ncc(-c2ccc3[nH]nc(-c4cc5c(-c6ccoc6)nccc5[nH]4)c3c2)cn1] | 505 |
| 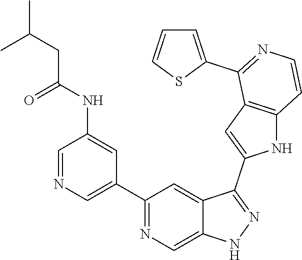 [SMILES: CC(C)CC(=O)Nc1cncc(-c2cc3c(-c4cc5c(-c6cccs6)nccc5[nH]4)n[nH]c3cn2)c1] | 506 |
| 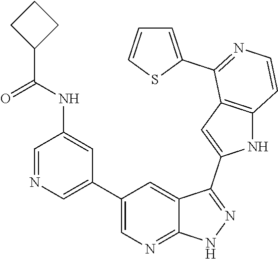 [SMILES: O=C(Nc1cncc(-c2cnc3[nH]nc(-c4cc5c(-c6cccs6)nccc5[nH]4)c3c2)c1)C1CCC1] | 507 |
| 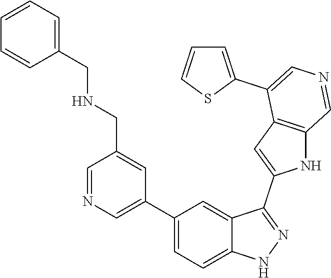 [SMILES: c1ccc(CNCc2cncc(-c3ccc4[nH]nc(-c5cc6c(-c7cccs7)cncc6[nH]5)c4c3)c2)cc1] | 508 |
| 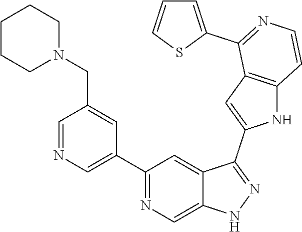 [SMILES: c1csc(-c2nccc3[nH]c(-c4n[nH]c5cnc(-c6cncc(CN7CCCCC7)c6)cc45)cc23)c1] | 509 |
| 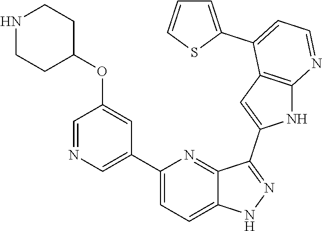 [SMILES: c1csc(-c2ccnc3[nH]c(-c4n[nH]c5ccc(-c6cncc(OC7CCNCC7)c6)nc45)cc23)c1] | 510 |
| 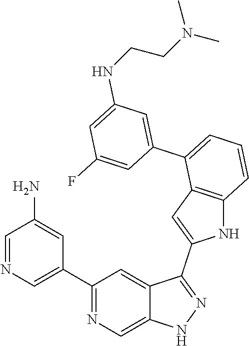 [SMILES: CN(C)CCNc1cc(F)cc(-c2cccc3[nH]c(-c4n[nH]c5cnc(-c6cncc(N)c6)cc45)cc23)c1] | 511 |
| 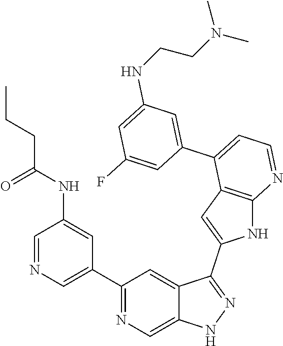 [SMILES: CCCC(=O)Nc1cncc(-c2cc3c(-c4cc5c(-c6cc(F)cc(NCCN(C)C)c6)ccnc5[nH]4)n[nH]c3cn2)c1] | 512 |
| 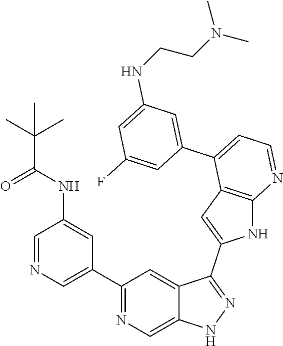 [SMILES: CN(C)CCNc1cc(F)cc(-c2ccnc3[nH]c(-c4n[nH]c5cnc(-c6cncc(NC(=O)C(C)(C)C)c6)cc45)cc23)c1] | 513 |
| 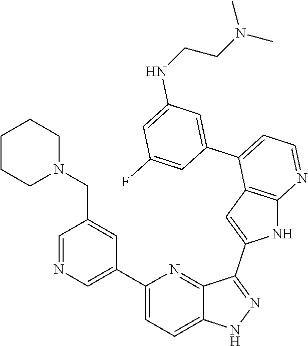 [SMILES: CN(C)CCNc1cc(F)cc(-c2ccnc3[nH]c(-c4n[nH]c5ccc(-c6cncc(CN7CCCCC7)c6)nc45)cc23)c1] | 514 |
| 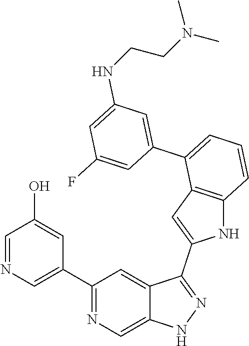 [SMILES: CN(C)CCNc1cc(F)cc(-c2cccc3[nH]c(-c4n[nH]c5cnc(-c6cncc(O)c6)cc45)cc23)c1] | 515 |
| 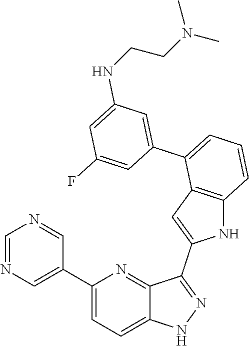 [SMILES: CN(C)CCNc1cc(F)cc(-c2cccc3[nH]c(-c4n[nH]c5ccc(-c6cncnc6)nc45)cc23)c1] | 516 |
| 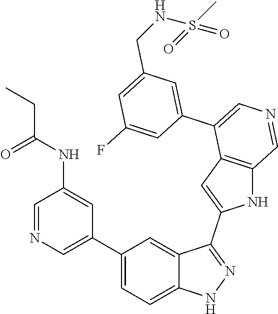 [SMILES: CCC(=O)Nc1cncc(-c2ccc3[nH]nc(-c4cc5c(-c6cc(F)cc(CNS(C)(=O)=O)c6)cncc5[nH]4)c3c2)c1] | 517 |
| 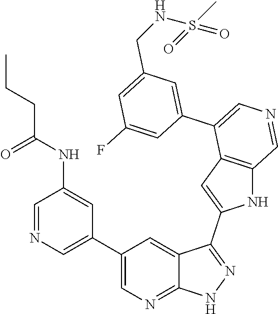 [SMILES: CCCC(=O)Nc1cncc(-c2cnc3[nH]nc(-c4cc5c(-c6cc(F)cc(CNS(C)(=O)=O)c6)cncc5[nH]4)c3c2)c1] | 518 |
| 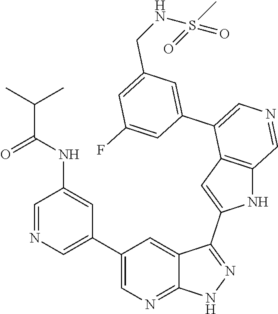 [SMILES: CC(C)C(=O)Nc1cncc(-c2cnc3[nH]nc(-c4cc5c(-c6cc(F)cc(CNS(C)(=O)=O)c6)cncc5[nH]4)c3c2)c1] | 519 |
| 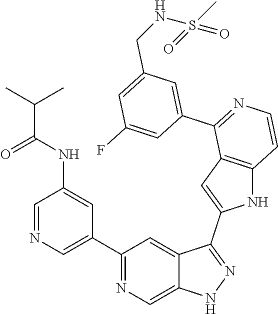 [SMILES: CC(C)C(=O)Nc1cncc(-c2cc3c(-c4cc5c(-c6cc(F)cc(CNS(C)(=O)=O)c6)nccc5[nH]4)n[nH]c3cn2)c1] | 520 |
| 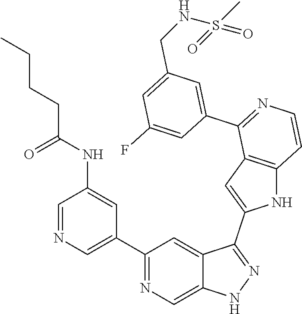 [SMILES: CCCCC(=O)Nc1cncc(-c2cc3c(-c4cc5c(-c6cc(F)cc(CNS(C)(=O)=O)c6)nccc5[nH]4)n[nH]c3cn2)c1] | 521 |
| 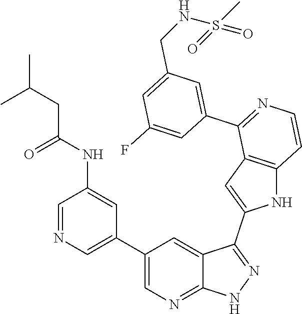 [SMILES: CC(C)CC(=O)Nc1cncc(-c2cnc3[nH]nc(-c4cc5c(-c6cc(F)cc(CNS(C)(=O)=O)c6)nccc5[nH]4)c3c2)c1] | 522 |
| 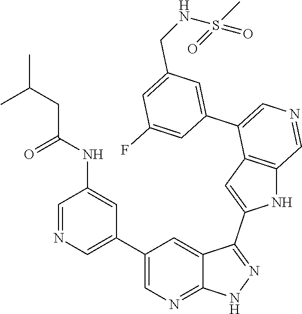 [SMILES: CC(C)CC(=O)Nc1cncc(-c2cnc3[nH]nc(-c4cc5c(-c6cc(F)cc(CNS(C)(=O)=O)c6)cncc5[nH]4)c3c2)c1] | 523 |
| 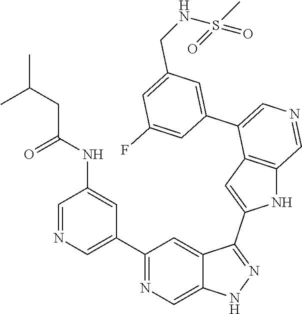 [SMILES: CC(C)CC(=O)Nc1cncc(-c2cc3c(-c4cc5c(-c6cc(F)cc(CNS(C)(=O)=O)c6)cncc5[nH]4)n[nH]c3cn2)c1] | 524 |
| 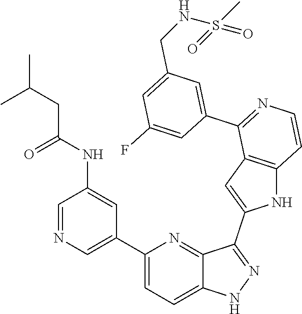 [SMILES: CC(C)CC(=O)Nc1cncc(-c2ccc3[nH]nc(-c4cc5c(-c6cc(F)cc(CNS(C)(=O)=O)c6)nccc5[nH]4)c3n2)c1] | 525 |
| 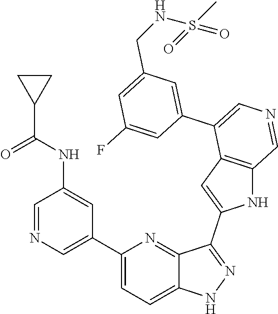 [SMILES: CS(=O)(=O)NCc1cc(F)cc(-c2cncc3[nH]c(-c4n[nH]c5ccc(-c6cncc(NC(=O)C7CC7)c6)nc45)cc23)c1] | 526 |
| 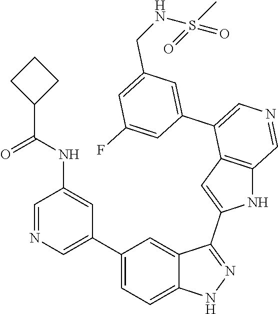 [SMILES: CS(=O)(=O)NCc1cc(F)cc(-c2cncc3[nH]c(-c4n[nH]c5ccc(-c6cncc(NC(=O)C7CCC7)c6)cc45)cc23)c1] | 527 |
| 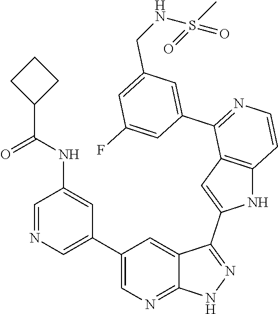 [SMILES: CS(=O)(=O)NCc1cc(F)cc(-c2nccc3[nH]c(-c4n[nH]c5ncc(-c6cncc(NC(=O)C7CCC7)c6)cc45)cc23)c1] | 528 |
| 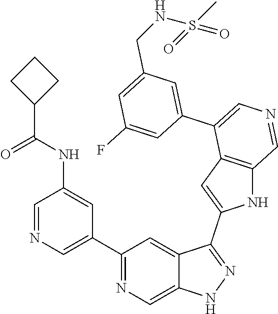 [SMILES: CS(=O)(=O)NCc1cc(F)cc(-c2cncc3[nH]c(-c4n[nH]c5cnc(-c6cncc(NC(=O)C7CCC7)c6)cc45)cc23)c1] | 529 |
| 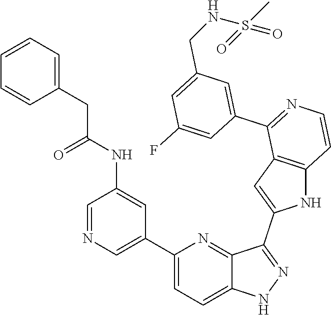 [SMILES: CS(=O)(=O)NCc1cc(F)cc(-c2nccc3[nH]c(-c4n[nH]c5ccc(-c6cncc(NC(=O)Cc7ccccc7)c6)nc45)cc23)c1] | 530 |
| 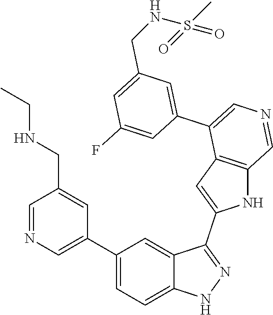 [SMILES: CCNCc1cncc(-c2ccc3[nH]nc(-c4cc5c(-c6cc(F)cc(CNS(C)(=O)=O)c6)cncc5[nH]4)c3c2)c1] | 531 |
| 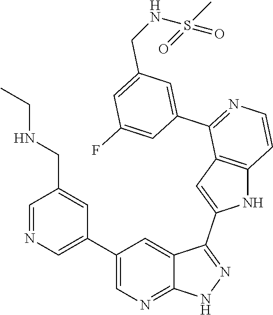 [SMILES: CCNCc1cncc(-c2cnc3[nH]nc(-c4cc5c(-c6cc(F)cc(CNS(C)(=O)=O)c6)nccc5[nH]4)c3c2)c1] | 532 |
| 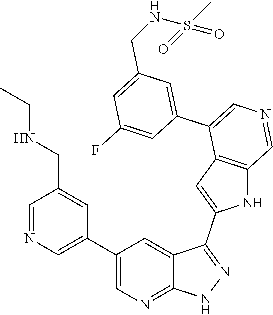 [SMILES: CCNCc1cncc(-c2cnc3[nH]nc(-c4cc5c(-c6cc(F)cc(CNS(C)(=O)=O)c6)cncc5[nH]4)c3c2)c1] | 533 |
| 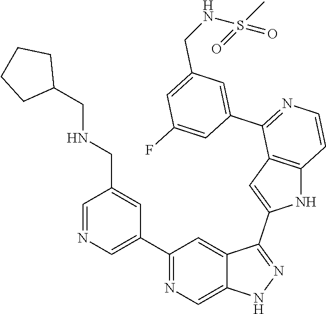 [SMILES: CS(=O)(=O)NCc1cc(F)cc(-c2nccc3[nH]c(-c4n[nH]c5cnc(-c6cncc(CNCC7CCCC7)c6)cc45)cc23)c1] | 534 |
| 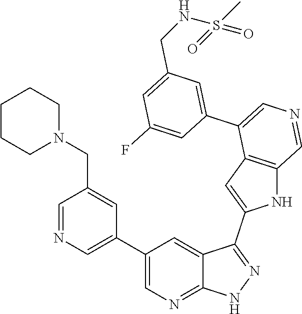 [SMILES: CS(=O)(=O)NCc1cc(F)cc(-c2cncc3[nH]c(-c4n[nH]c5ncc(-c6cncc(CN7CCCCC7)c6)cc45)cc23)c1] | 535 |
| 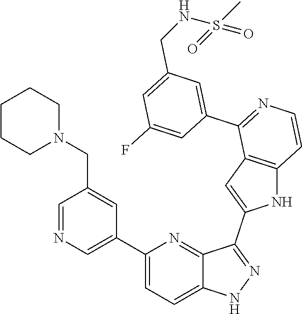 [SMILES: CS(=O)(=O)NCc1cc(F)cc(-c2nccc3[nH]c(-c4n[nH]c5ccc(-c6cncc(CN7CCCCC7)c6)nc45)cc23)c1] | 536 |
| 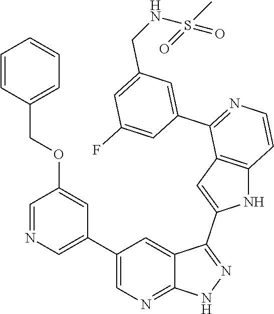 [SMILES: CS(=O)(=O)NCc1cc(F)cc(-c2nccc3[nH]c(-c4n[nH]c5ncc(-c6cncc(OCc7ccccc7)c6)cc45)cc23)c1] | 537 |
| 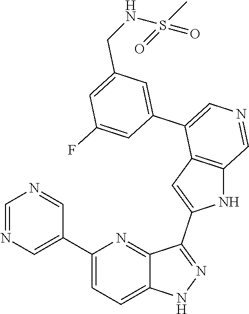 [SMILES: CS(=O)(=O)NCc1cc(F)cc(-c2cncc3[nH]c(-c4n[nH]c5ccc(-c6cncnc6)nc45)cc23)c1] | 538 |
| 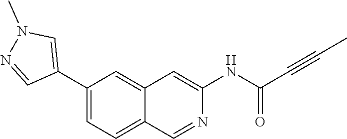 [SMILES: CC#CC(=O)Nc1cc2cc(-c3cnn(C)c3)ccc2cn1] | 539 |
| 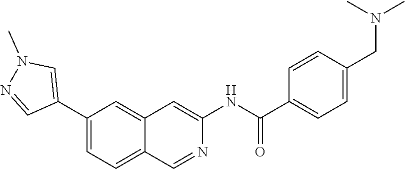 [SMILES: CN(C)Cc1ccc(C(=O)Nc2cc3cc(-c4cnn(C)c4)ccc3cn2)cc1] | 540 |
| 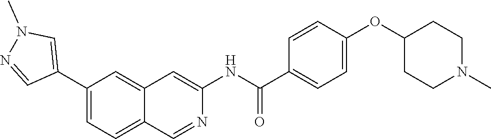 [SMILES: CN1CCC(Oc2ccc(C(=O)Nc3cc4cc(-c5cnn(C)c5)ccc4cn3)cc2)CC1] | 541 |
| 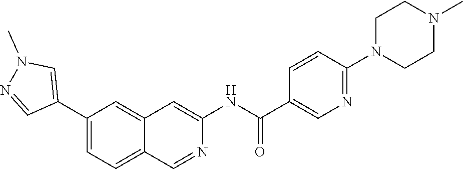 [SMILES: CN1CCN(c2ccc(C(=O)Nc3cc4cc(-c5cnn(C)c5)ccc4cn3)cn2)CC1] | 542 |
| 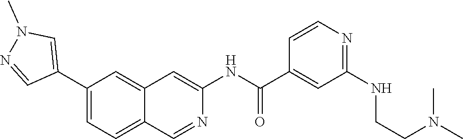 [SMILES: CN(C)CCNc1cc(C(=O)Nc2cc3cc(-c4cnn(C)c4)ccc3cn2)ccn1] | 543 |
| 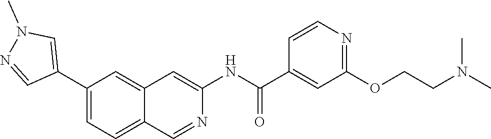 [SMILES: CN(C)CCOc1cc(C(=O)Nc2cc3cc(-c4cnn(C)c4)ccc3cn2)ccn1] | 544 |
| 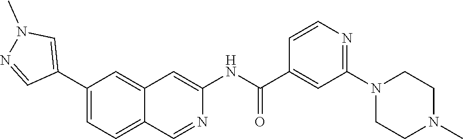 [SMILES: CN1CCN(c2cc(C(=O)Nc3cc4cc(-c5cnn(C)c5)ccc4cn3)ccn2)CC1] | 545 |
| 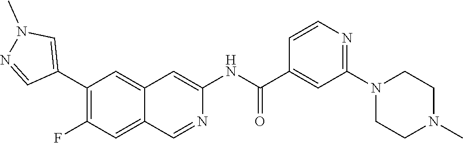 [SMILES: CN1CCN(c2cc(C(=O)Nc3cc4cc(-c5cnn(C)c5)c(F)cc4cn3)ccn2)CC1] | 546 |
| 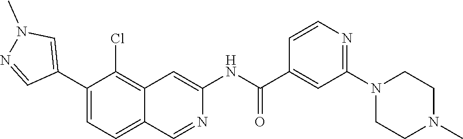 [SMILES: CN1CCN(c2cc(C(=O)Nc3cc4c(Cl)c(-c5cnn(C)c5)ccc4cn3)ccn2)CC1] | 547 |
| 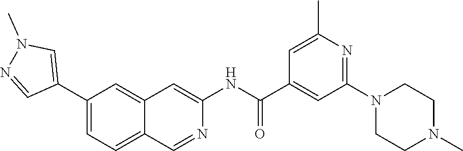 [SMILES: Cc1cc(C(=O)Nc2cc3cc(-c4cnn(C)c4)ccc3cn2)cc(N2CCN(C)CC2)n1] | 548 |
| 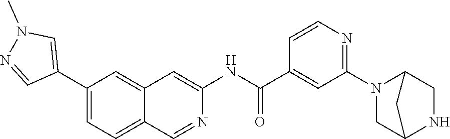 [SMILES: Cn1cc(-c2ccc3cnc(NC(=O)c4ccnc(N5CC6CC5CN6)c4)cc3c2)cn1] | 549 |
| 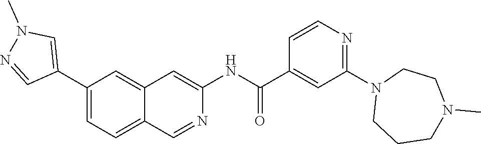 [SMILES: CN1CCCN(c2cc(C(=O)Nc3cc4cc(-c5cnn(C)c5)ccc4cn3)ccn2)CC1] | 550 |
| 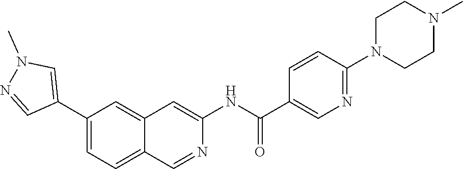 [SMILES: CN1CCN(c2ccc(C(=O)Nc3cc4cc(-c5cnn(C)c5)ccc4cn3)cn2)CC1] | 551 |
| 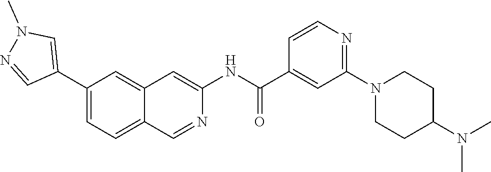 [SMILES: CN(C)C1CCN(c2cc(C(=O)Nc3cc4cc(-c5cnn(C)c5)ccc4cn3)ccn2)CC1] | 552 |
| 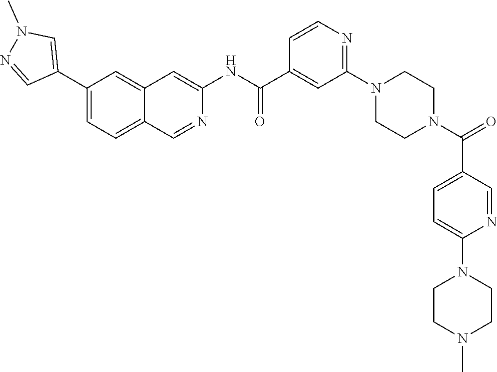 [SMILES: CN1CCN(c2ccc(C(=O)N3CCN(c4cc(C(=O)Nc5cc6cc(-c7cnn(C)c7)ccc6cn5)ccn4)CC3)cn2)CC1] | 553 |
| 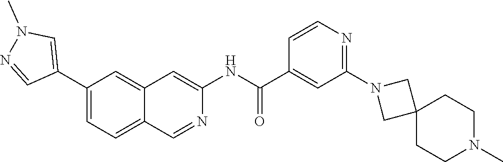 [SMILES: CN1CCC2(CC1)CN(c1cc(C(=O)Nc3cc4cc(-c5cnn(C)c5)ccc4cn3)ccn1)C2] | 554 |
| 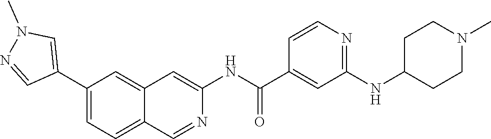 [SMILES: CN1CCC(Nc2cc(C(=O)Nc3cc4cc(-c5cnn(C)c5)ccc4cn3)ccn2)CC1] | 555 |
| 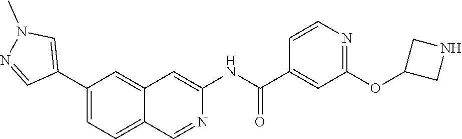 [SMILES: Cn1cc(-c2ccc3cnc(NC(=O)c4ccnc(OC5CNC5)c4)cc3c2)cn1] | 556 |
| 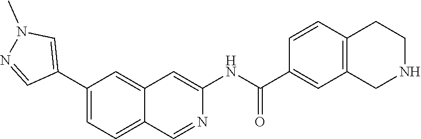 [SMILES: Cn1cc(-c2ccc3cnc(NC(=O)c4ccc5c(c4)CNCC5)cc3c2)cn1] | 557 |
| 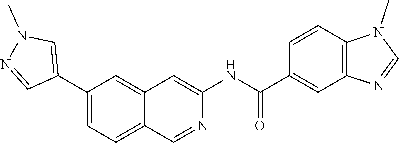 [SMILES: Cn1cc(-c2ccc3cnc(NC(=O)c4ccc5c(c4)ncn5C)cc3c2)cn1] | 558 |
| 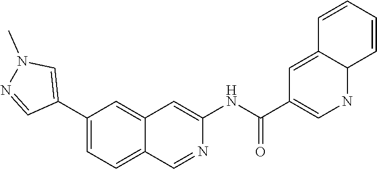 [SMILES: Cn1cc(-c2ccc3cnc(NC(=O)C4=CNC5C=CC=CC5=C4)cc3c2)cn1] | 559 |
| 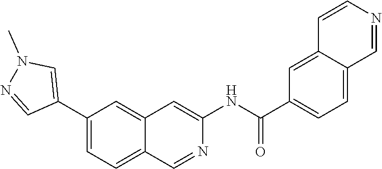 [SMILES: Cn1cc(-c2ccc3cnc(NC(=O)c4ccc5cnccc5c4)cc3c2)cn1] | 560 |
| 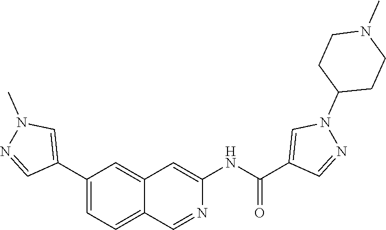 [SMILES: CN1CCC(n2cc(C(=O)Nc3cc4cc(-c5cnn(C)c5)ccc4cn3)cn2)CC1] | 561 |
| 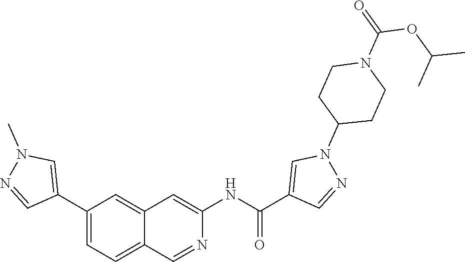 [SMILES: CC(C)OC(=O)N1CCC(n2cc(C(=O)Nc3cc4cc(-c5cnn(C)c5)ccc4cn3)cn2)CC1] | 562 |
| 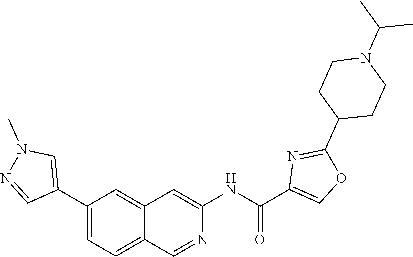 [SMILES: CC(C)N1CCC(c2nc(C(=O)Nc3cc4cc(-c5cnn(C)c5)ccc4cn3)co2)CC1] | 563 |
| 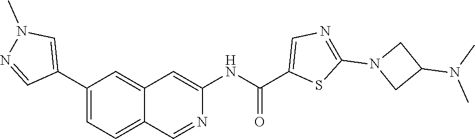 [SMILES: CN(C)C1CN(c2ncc(C(=O)Nc3cc4cc(-c5cnn(C)c5)ccc4cn3)s2)C1] | 564 |
| 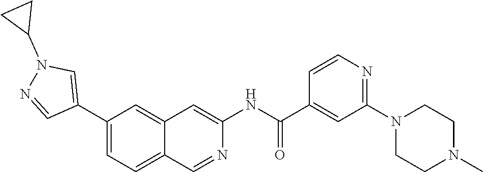 [SMILES: CN1CCN(c2cc(C(=O)Nc3cc4cc(-c5cnn(C6CC6)c5)ccc4cn3)ccn2)CC1] | 565 |
| 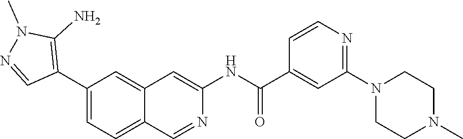 [SMILES: CN1CCN(c2cc(C(=O)Nc3cc4cc(-c5cnn(C)c5N)ccc4cn3)ccn2)CC1] | 566 |
| 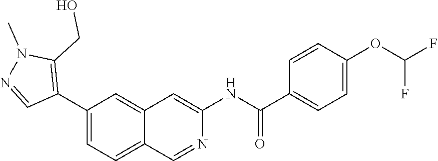 [SMILES: Cn1ncc(-c2ccc3cnc(NC(=O)c4ccc(OC(F)F)cc4)cc3c2)c1CO] | 567 |
| 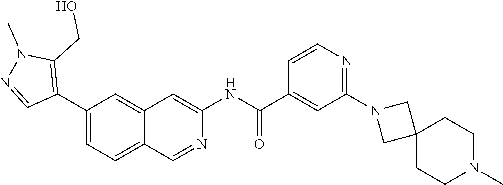 [SMILES: CN1CCC2(CC1)CN(c1cc(C(=O)Nc3cc4cc(-c5cnn(C)c5CO)ccc4cn3)ccn1)C2] | 568 |
| 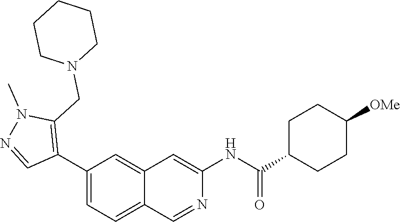 [SMILES: CO[C@H]1CC[C@H](C(=O)Nc2cc3cc(-c4cnn(C)c4CN4CCCCC4)ccc3cn2)CC1] | 569 |
| 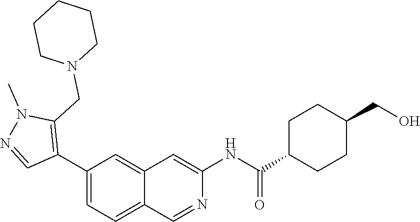 [SMILES: Cn1ncc(-c2ccc3cnc(NC(=O)[C@H]4CC[C@H](CO)CC4)cc3c2)c1CN1CCCCC1] | 570 |
| 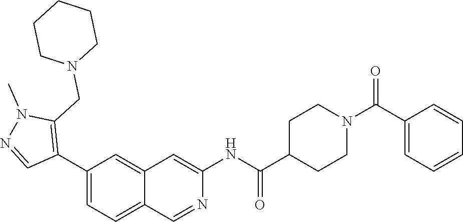 [SMILES: Cn1ncc(-c2ccc3cnc(NC(=O)C4CCN(C(=O)c5ccccc5)CC4)cc3c2)c1CN1CCCCC1] | 571 |
| 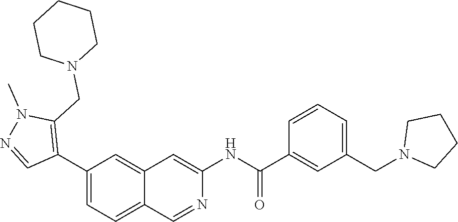 [SMILES: Cn1ncc(-c2ccc3cnc(NC(=O)c4cccc(CN5CCCC5)c4)cc3c2)c1CN1CCCCC1] | 572 |
| 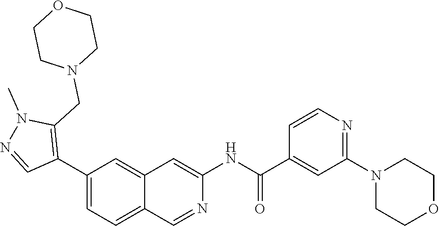 [SMILES: Cn1ncc(-c2ccc3cnc(NC(=O)c4ccnc(N5CCOCC5)c4)cc3c2)c1CN1CCOCC1] | 573 |
| 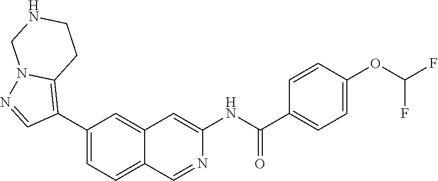 [SMILES: O=C(Nc1cc2cc(-c3cnn4c3CCNC4)ccc2cn1)c1ccc(OC(F)F)cc1] | 574 |
| 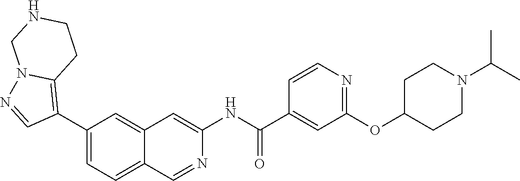 [SMILES: CC(C)N1CCC(Oc2cc(C(=O)Nc3cc4cc(-c5cnn6c5CCNC6)ccc4cn3)ccn2)CC1] | 575 |
| 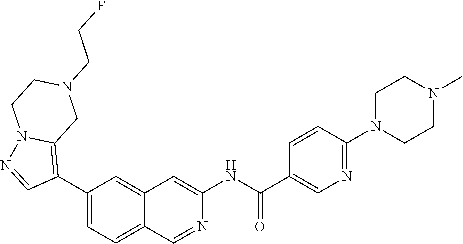 [SMILES: CN1CCN(c2ccc(C(=O)Nc3cc4cc(-c5cnn6c5CN(CCF)CC6)ccc4cn3)cn2)CC1] | 576 |
| 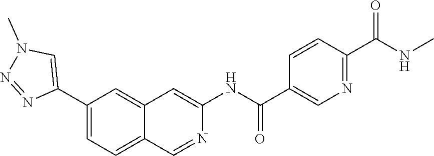 [SMILES: CNC(=O)c1ccc(C(=O)Nc2cc3cc(-c4cn(C)nn4)ccc3cn2)cn1] | 577 |
| 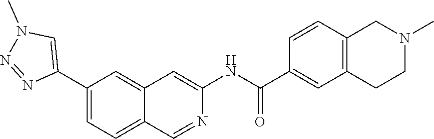 [SMILES: CN1CCc2cc(C(=O)Nc3cc4cc(-c5cn(C)nn5)ccc4cn3)ccc2C1] | 578 |
| 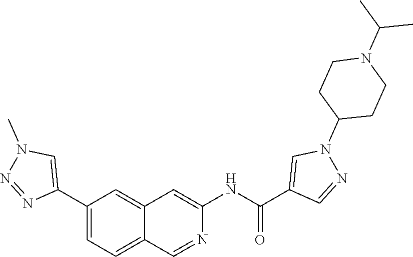 [SMILES: CC(C)N1CCC(n2cc(C(=O)Nc3cc4cc(-c5cn(C)nn5)ccc4cn3)cn2)CC1] | 579 |
| 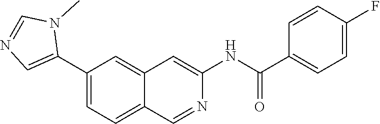 [SMILES: Cn1cncc1-c1ccc2cnc(NC(=O)c3ccc(F)cc3)cc2c1] | 580 |
| 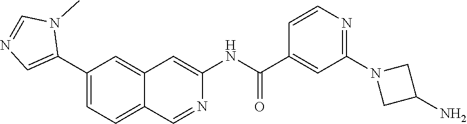 [SMILES: Cn1cncc1-c1ccc2cnc(NC(=O)c3ccnc(N4CC(N)C4)c3)cc2c1] | 581 |
| 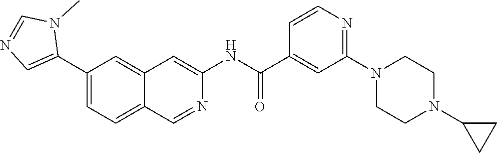 [SMILES: Cn1cncc1-c1ccc2cnc(NC(=O)c3ccnc(N4CCN(C5CC5)CC4)c3)cc2c1] | 582 |
| 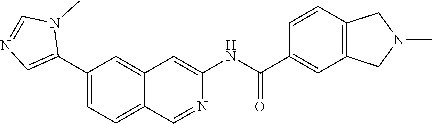 [SMILES: CN1Cc2ccc(C(=O)Nc3cc4cc(-c5cncn5C)ccc4cn3)cc2C1] | 583 |
| 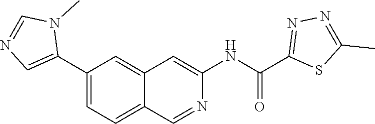 [SMILES: Cc1nnc(C(=O)Nc2cc3cc(-c4cncn4C)ccc3cn2)s1] | 574 |
| 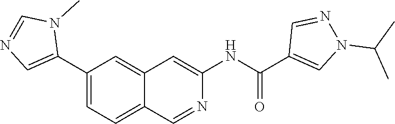 [SMILES: CC(C)n1cc(C(=O)Nc2cc3cc(-c4cncn4C)ccc3cn2)cn1] | 585 |
| 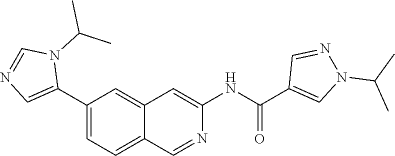 [SMILES: CC(C)n1cc(C(=O)Nc2cc3cc(-c4cncn4C(C)C)ccc3cn2)cn1] | 586 |
| 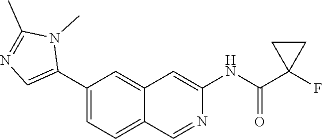 [SMILES: Cc1ncc(-c2ccc3cnc(NC(=O)C4(F)CC4)cc3c2)n1C] | 587 |
| 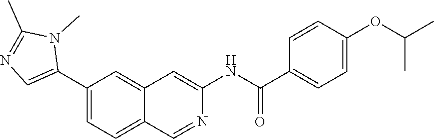 [SMILES: Cc1ncc(-c2ccc3cnc(NC(=O)c4ccc(OC(C)C)cc4)cc3c2)n1C] | 588 |
| 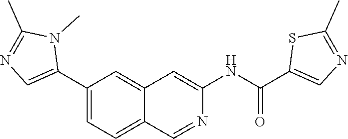 [SMILES: Cc1ncc(C(=O)Nc2cc3cc(-c4cnc(C)n4C)ccc3cn2)s1] | 589 |
| 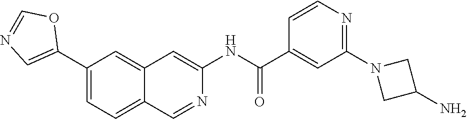 [SMILES: NC1CN(c2cc(C(=O)Nc3cc4cc(-c5cnco5)ccc4cn3)ccn2)C1] | 590 |
| 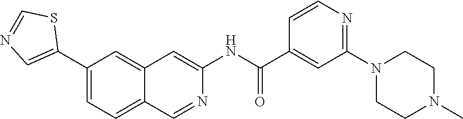 [SMILES: CN1CCN(c2cc(C(=O)Nc3cc4cc(-c5cncs5)ccc4cn3)ccn2)CC1] | 591 |
| 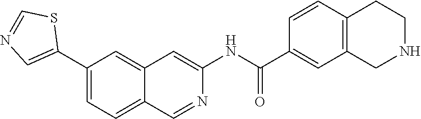 [SMILES: O=C(Nc1cc2cc(-c3cncs3)ccc2cn1)c1ccc2c(c1)CNCC2] | 592 |
| 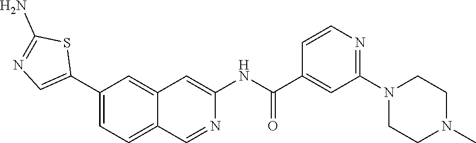 [SMILES: CN1CCN(c2cc(C(=O)Nc3cc4cc(-c5cnc(N)s5)ccc4cn3)ccn2)CC1] | 593 |
| 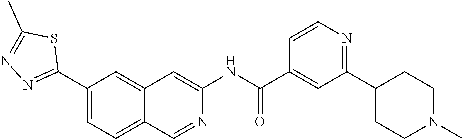 [SMILES: Cc1nnc(-c2ccc3cnc(NC(=O)c4ccnc(C5CCN(C)CC5)c4)cc3c2)s1] | 594 |
| 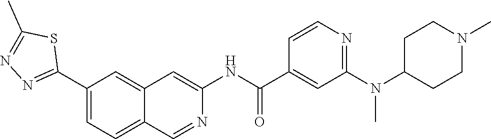 [SMILES: Cc1nnc(-c2ccc3cnc(NC(=O)c4ccnc(N(C)C5CCN(C)CC5)c4)cc3c2)s1] | 595 |
| 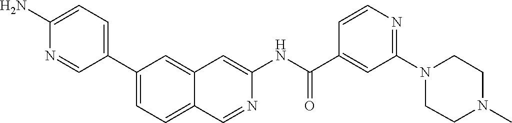 [SMILES: CN1CCN(c2cc(C(=O)Nc3cc4cc(-c5ccc(N)nc5)ccc4cn3)ccn2)CC1] | 596 |
| 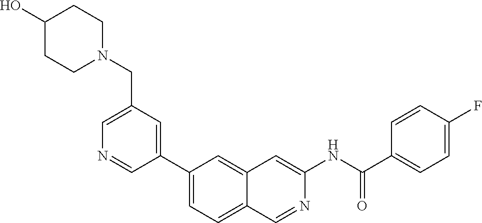 [SMILES: O=C(Nc1cc2cc(-c3cncc(CN4CCC(O)CC4)c3)ccc2cn1)c1ccc(F)cc1] | 597 |
| 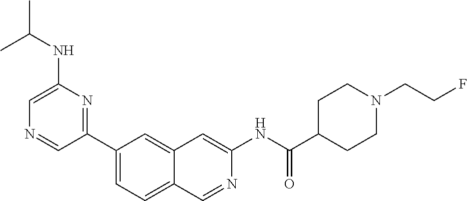 [SMILES: CC(C)Nc1cncc(-c2ccc3cnc(NC(=O)C4CCN(CCF)CC4)cc3c2)n1] | 598 |
| 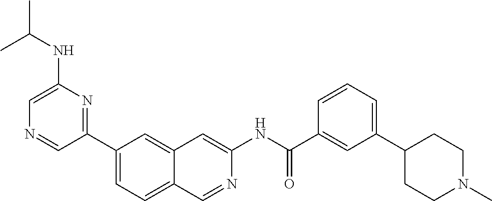 [SMILES: CC(C)Nc1cncc(-c2ccc3cnc(NC(=O)c4cccc(C5CCN(C)CC5)c4)cc3c2)n1] | 599 |
| 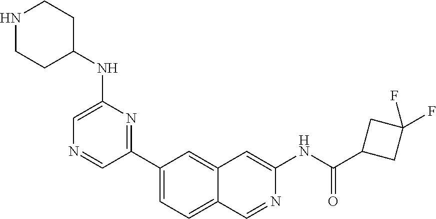 [SMILES: O=C(Nc1cc2cc(-c3cncc(NC4CCNCC4)n3)ccc2cn1)C1CC(F)(F)C1] | 600 |
| 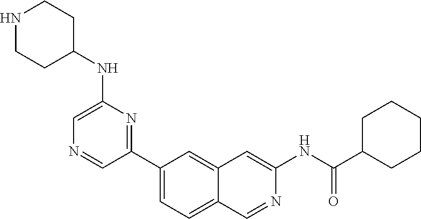 [SMILES: O=C(Nc1cc2cc(-c3cncc(NC4CCNCC4)n3)ccc2cn1)C1CCCCC1] | 601 |
| 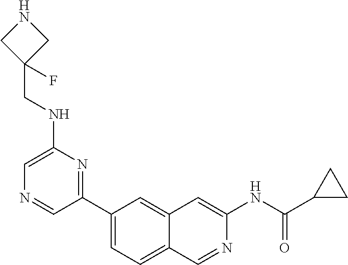 [SMILES: O=C(Nc1cc2cc(-c3cncc(NCC4(F)CNC4)n3)ccc2cn1)C1CC1] | 602 |
| 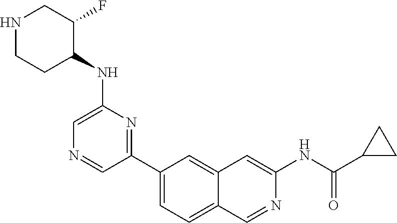 | 603 |
| 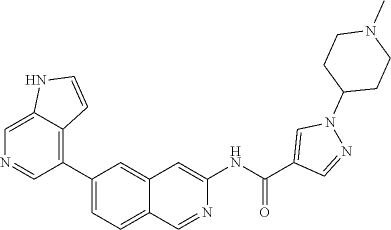 [SMILES: CN1CCC(n2cc(C(=O)Nc3cc4cc(-c5cncc6[nH]ccc56)ccc4cn3)cn2)CC1] | 604 |
| 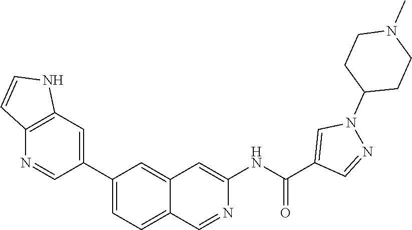 [SMILES: CN1CCC(n2cc(C(=O)Nc3cc4cc(-c5cnc6cc[nH]c6c5)ccc4cn3)cn2)CC1] | 605 |
| 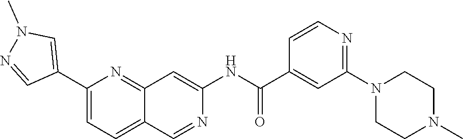 [SMILES: CN1CCN(c2cc(C(=O)Nc3cc4nc(-c5cnn(C)c5)ccc4cn3)ccn2)CC1] | 606 |
| 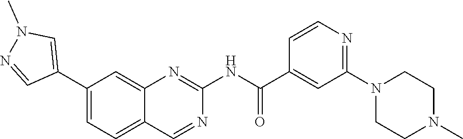 [SMILES: CN1CCN(c2cc(C(=O)Nc3ncc4ccc(-c5cnn(C)c5)cc4n3)ccn2)CC1] | 607 |
| 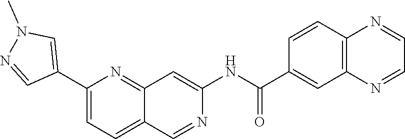 [SMILES: Cn1cc(-c2ccc3cnc(NC(=O)c4ccc5nccnc5c4)cc3n2)cn1] | 608 |
| 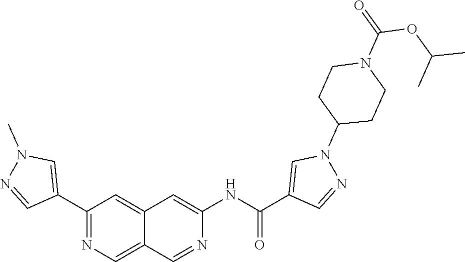 [SMILES: CC(C)OC(=O)N1CCC(n2cc(C(=O)Nc3cc4cc(-c5cnn(C)c5)ncc4cn3)cn2)CC1] | 609 |
| 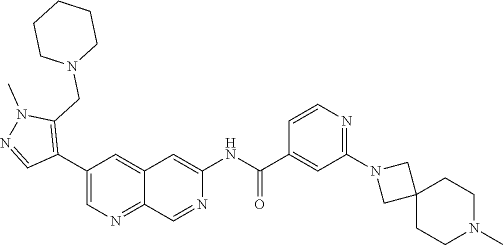 [SMILES: CN1CCC2(CC1)CN(c1cc(C(=O)Nc3cc4cc(-c5cnn(C)c5CN5CCCCC5)cnc4cn3)ccn1)C2] | 610 |
| 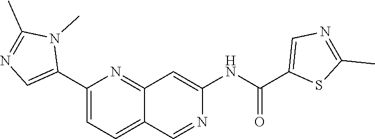 [SMILES: Cc1ncc(C(=O)Nc2cc3nc(-c4cnc(C)n4C)ccc3cn2)s1] | 611 |
| 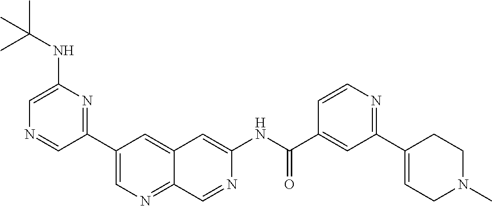 [SMILES: CN1CC=C(c2cc(C(=O)Nc3cc4cc(-c5cncc(NC(C)(C)C)n5)cnc4cn3)ccn2)CC1] | 612 |
| 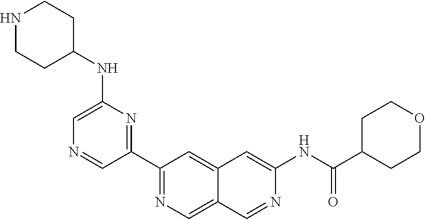 [SMILES: O=C(Nc1cc2cc(-c3cncc(NC4CCNCC4)n3)ncc2cn1)C1CCOCC1] | 613 |
| 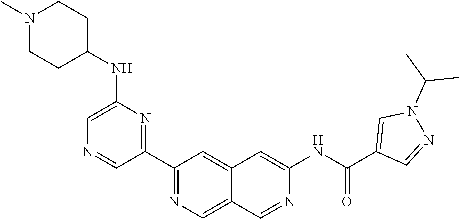 [SMILES: CC(C)n1cc(C(=O)Nc2cc3cc(-c4cncc(NC5CCN(C)CC5)n4)ncc3cn2)cn1] | 614 |
| 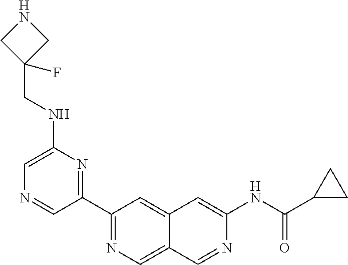 [SMILES: O=C(Nc1cc2cc(-c3cncc(NCC4(F)CNC4)n3)ncc2cn1)C1CC1] | 615 |
| 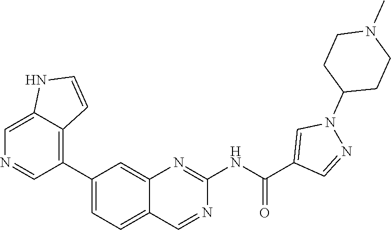 [SMILES: CN1CCC(n2cc(C(=O)Nc3ncc4ccc(-c5cncc6[nH]ccc56)cc4n3)cn2)CC1] | 616 |
| 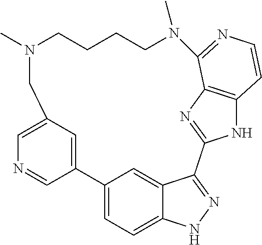 [SMILES: CN1CCCCN(C)c2nccc3[nH]c(nc23)-c2n[nH]c3ccc(cc23)-c2cncc(c2)C1] | 617 |
| 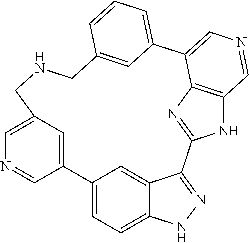 [SMILES: c1cc2cc(c1)-c1cncc3[nH]c(nc13)-c1n[nH]c3ccc(cc13)-c1cncc(c1)CNC2] | 618 |
| 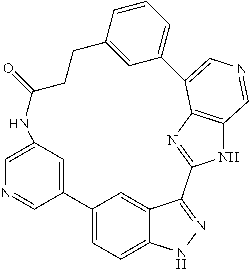 [SMILES: O=C1CCc2cccc(c2)-c2cncc3[nH]c(nc23)-c2n[nH]c3ccc(cc23)-c2cncc(c2)N1] | 619 |
| 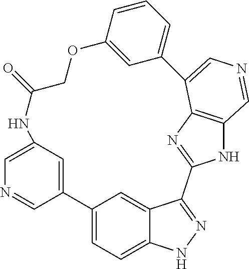 [SMILES: O=C1COc2cccc(c2)-c2cncc3[nH]c(nc23)-c2n[nH]c3ccc(cc23)-c2cncc(c2)N1] | 620 |
| 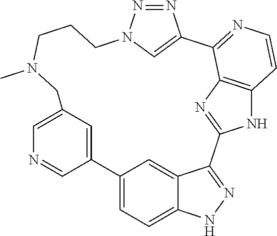 [SMILES: CN1CCCn2cc(nn2)-c2nccc3[nH]c(nc23)-c2n[nH]c3ccc(cc23)-c2cncc(c2)C1] | 621 |
| 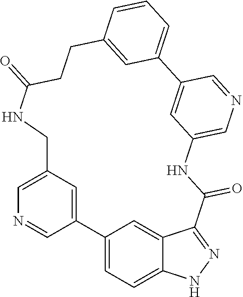 [SMILES: O=C1CCc2cccc(c2)-c2cncc(c2)NC(=O)c2n[nH]c3ccc(cc23)-c2cncc(c2)CN1] | 622 |
| 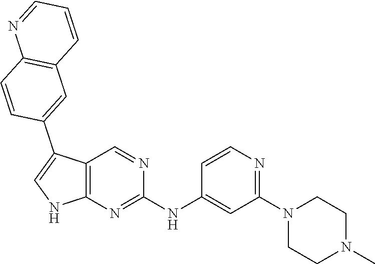 [SMILES: CN1CCN(c2cc(Nc3ncc4c(-c5ccc6ncccc6c5)c[nH]c4n3)ccn2)CC1] | 623 |
| 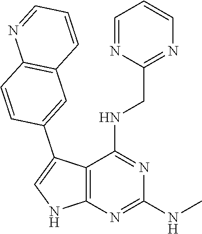 [SMILES: CNc1nc(NCc2ncccn2)c2c(-c3ccc4ncccc4c3)c[nH]c2n1] | 624 |
| 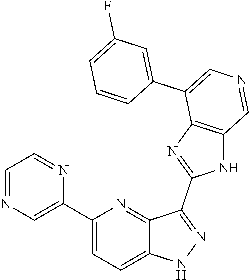 [SMILES: Fc1cccc(-c2cncc3[nH]c(-c4n[nH]c5ccc(-c6cnccn6)nc45)nc23)c1] | 625 |
| 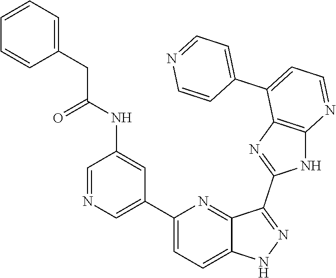 [SMILES: O=C(Cc1ccccc1)Nc1cncc(-c2ccc3[nH]nc(-c4nc5c(-c6ccncc6)ccnc5[nH]4)c3n2)c1] | 626 |
| 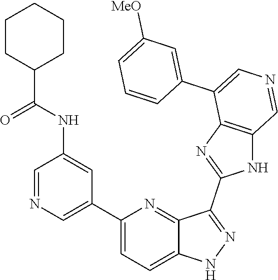 [SMILES: COc1cccc(-c2cncc3[nH]c(-c4n[nH]c5ccc(-c6cncc(NC(=O)C7CCCCC7)c6)nc45)nc23)c1] | 627 |
| 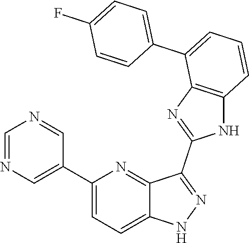 [SMILES: Fc1ccc(-c2cccc3[nH]c(-c4n[nH]c5ccc(-c6cncnc6)nc45)nc23)cc1] | 628 |
| 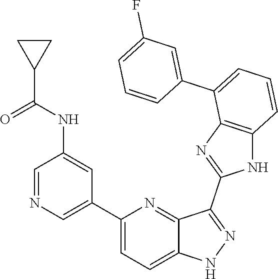 [SMILES: O=C(Nc1cncc(-c2ccc3[nH]nc(-c4nc5c(-c6cccc(F)c6)cccc5[nH]4)c3n2)c1)C1CC1] | 629 |
| 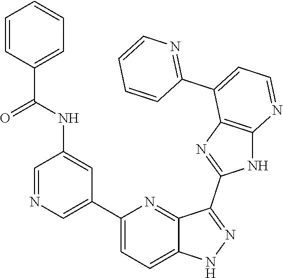 [SMILES: O=C(Nc1cncc(-c2ccc3[nH]nc(-c4nc5c(-c6ccccn6)ccnc5[nH]4)c3n2)c1)c1ccccc1] | 630 |
| 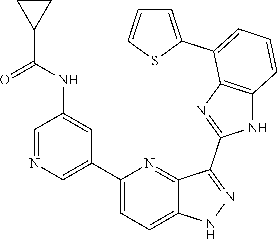 [SMILES: O=C(Nc1cncc(-c2ccc3[nH]nc(-c4nc5c(-c6cccs6)cccc5[nH]4)c3n2)c1)C1CC1] | 631 |
| 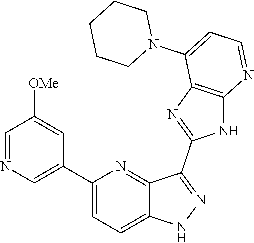 [SMILES: COc1cncc(-c2ccc3[nH]nc(-c4nc5c(N6CCCCC6)ccnc5[nH]4)c3n2)c1] | 632 |
| 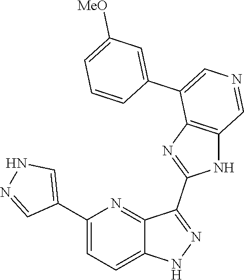 [SMILES: COc1cccc(-c2cncc3[nH]c(-c4n[nH]c5ccc(-c6cn[nH]c6)nc45)nc23)c1] | 633 |
| 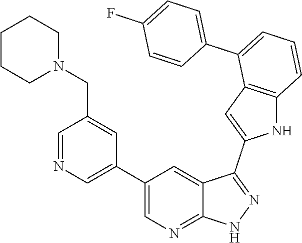 [SMILES: Fc1ccc(-c2cccc3[nH]c(-c4n[nH]c5ncc(-c6cncc(CN7CCCCC7)c6)cc45)cc23)cc1] | 634 |
| 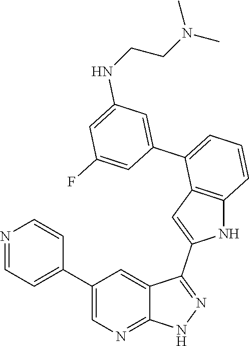 [SMILES: CN(C)CCNc1cc(F)cc(-c2cccc3[nH]c(-c4n[nH]c5ncc(-c6ccncc6)cc45)cc23)c1] | 635 |
| 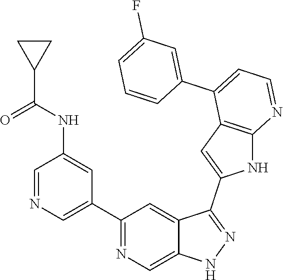 [SMILES: O=C(Nc1cncc(-c2cc3c(-c4cc5c(-c6cccc(F)c6)ccnc5[nH]4)n[nH]c3cn2)c1)C1CC1] | 636 |
| 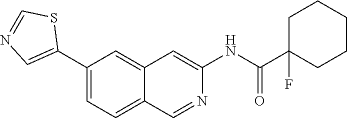 [SMILES: O=C(Nc1cc2cc(-c3cncs3)ccc2cn1)C1(F)CCCCC1] | 637 |
| 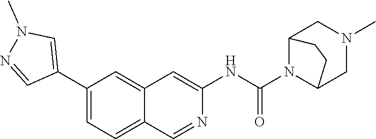 [SMILES: CN1CC2CCC(C1)N2C(=O)Nc1cc2cc(-c3cnn(C)c3)ccc2cn1] | 638 |
| 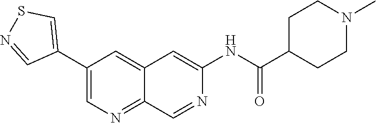 [SMILES: CN1CCC(C(=O)Nc2cc3cc(-c4cnsc4)cnc3cn2)CC1] | 639 |
| 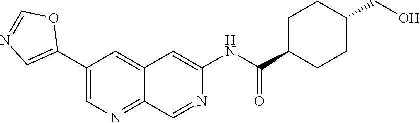 [SMILES: O=C(Nc1cc2cc(-c3cnco3)cnc2cn1)[C@H]1CC[C@H](CO)CC1] | 640 |
| 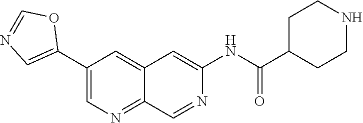 [SMILES: O=C(Nc1cc2cc(-c3cnco3)cnc2cn1)C1CCNCC1] | 641 |
| 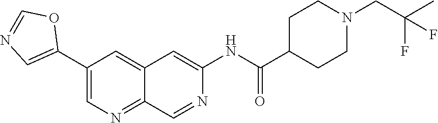 [SMILES: CC(F)(F)CN1CCC(C(=O)Nc2cc3cc(-c4cnco4)cnc3cn2)CC1] | 642 |

### Administration and Pharmaceutical Compositions

Also provided herein are compositions (e.g., pharmaceutical compositions) that include at least one dual CLK/DYRK inhibitor, or a pharmaceutically acceptable salt or solvate thereof, or a combination of a CLK inhibitor, or a pharmaceutically acceptable salt or solvate thereof, and DYRK inhibitor, or a pharmaceutically acceptable salt or solvate thereof, (e.g., any of the exemplary CLK or DYRK inhibitors described herein or known in the art) and instructions for performing any of the methods described herein. In some embodiments, the compositions (e.g., pharmaceutical compositions) can be disposed in a sterile vial or a pre-loaded syringe.

The term “administration” or “administering” refers to a method of providing a dosage of a compound or pharmaceutical composition to a vertebrate or invertebrate, including a mammal, a bird, a fish, or an amphibian, where the method is, e.g., orally, subcutaneously, intravenously, intralymphatically, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease. In some embodiments, the administration method includes oral or parenteral administration.

The term “subject” is defined herein to include animals such as mammals, including but not limited to, mice, rats, rabbits, dogs, cats, horses, goats, sheep, pigs, goats, cows, primates (e.g., humans), and the like. In some embodiments, the subject is a human. In some embodiments of any of the methods described herein, a subject may be referred to as a “patient.” In some embodiments of any of the methods described herein, the subject is 1 year old or older, 5 years old or older, 10 years old or older, 15 years old or older, 18 years old or older, 20 years old or older, 25 years old or older, 30 years old or older, 35 years old or older, 40 years old or older, 45 years old or older, 50 years old or older, 55 years old or older, 60 years old or older, 65 years old or older, 70 years old or older, 75 years old or older, 80 years old or older, 85 years old or older, 90 years old or older, 95 years old or older, 100 years old or older, or 105 years old or older.

In some embodiments, the compound(s) provided herein, for example, a first compound and a second compound, may be administered simultaneously or sequentially (in either order). The CLK inhibitor, or a pharmaceutically acceptable salt or solvate thereof, can be the first compound or the second compound. Likewise, the DYRK inhibitor, or a pharmaceutically acceptable salt or solvate thereof, can be the first compound or the second compound. For example, in some embodiments described herein, the CLK inhibitor, or a pharmaceutically acceptable salt or solvate thereof, may be administered first, followed by the DYRK inhibitor, or a pharmaceutically acceptable salt or solvate thereof. In other embodiments, the DYRK inhibitor, or a pharmaceutically acceptable salt or solvate thereof, may be administered first, followed by the CLK inhibitor, or a pharmaceutically acceptable salt or solvate thereof. Simultaneous administration refers to administration at substantially the same time. In some embodiments, the compound(s) provided herein, for example, a first compound and a second compound, are combined into a single formulation. Alternatively, the compound(s) provided herein, for example, a first compound and a second compound, may be formulated separately. In some embodiments, the compound(s) provided herein are administered parenterally, including intramuscularly, intraarticularly, periarticularly, intraspinally, intrasynovially, and epidurally. For example, the compound(s) can be injected locally at the site of the osteoarthritis (e.g., knee, hip, shoulder, etc.). Injections can occur at one or more locations surrounding the joint. In some embodiments, the injection is guided using an imaging method such as ultrasound. In some embodiments, administration (e.g., injection) of a first compound and second compound is preceded or combined with a local anesthetic. In some embodiments, administration (e.g., injection) of a single compound is preceded or combined with a local anesthetic.

Compounds provided herein intended for pharmaceutical use may be administered as crystalline or amorphous products. Pharmaceutically acceptable compositions may include solid, semi solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants and the like, for prolonged and/or timed, pulsed administration at a predetermined rate.

In some embodiments, the compositions (e.g., pharmaceutical compositions) are formulated for different routes of administration (e.g., intravenous, intramuscular, subcutaneous, or intracranial). In some embodiments, the compositions (e.g., pharmaceutical compositions) can include a pharmaceutically acceptable salt (e.g., phosphate buffered saline). In some embodiments, the compositions (e.g., pharmaceutical compositions) can include an enantiomer, a diastereoisomer, or a tautomer. Single or multiple administrations of any of the pharmaceutical compositions described herein can be given to a subject depending on, for example: the dosage and frequency as required and tolerated by the subject. A dosage of the pharmaceutical composition comprising a first compound, wherein the first compound is a CLK inhibitor and a second compound, wherein the second compound is a DYRK inhibitor or a single compound, wherein the single compound is a dual CLK/DYRK inhibitor, or pharmaceutically acceptable salt or solvate of the first, second, or single compounds, to effectively treat or ameliorate conditions, diseases, or symptoms of osteoarthritis.

The compounds can be administered either alone or in combination with a conventional pharmaceutical carrier, excipient or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

Injectables can be prepared in conventional forms, either as liquid solutions, colloid, liposomes, complexes, coacervate or suspensions, as emulsions, or in solid forms suitable for reconstitution in liquid prior to injection. In some embodiments, the compositions are provided in unit dosage forms suitable for a single administration. In some embodiments, the compositions are provided in unit dosage forms suitable for twice a day administration. In some embodiments, the compositions are provided in unit dosage forms suitable for three times a day administration.

In some embodiments, the compositions are provided in dosage forms suitable for continuous dosage by intravenous infusion over a period of about 1-96 hours. In some embodiments, the compositions are provided in dosage forms suitable for continuous dosage by intravenous infusion over a period of about 1-72 hours. In some embodiments, the compositions are provided in dosage forms suitable for continuous dosage by intravenous infusion over a period of about 1-48 hours. In some embodiments, the compositions are provided in dosage forms suitable for continuous dosage by intravenous infusion over a period of about 1-24 hours. In some embodiments, the compositions are provided in dosage forms suitable for continuous dosage by intravenous infusion over a period of about 1-12 hours. In some embodiments, the compositions are provided in dosage forms suitable for continuous dosage by intravenous infusion over a period of about 1-6 hours. In some embodiments, the compositions are provided in dosage forms suitable for continuous dosage by intravenous infusion over a period of about 1-4 hours. In some embodiments, the compositions are provided in dosage forms suitable for continuous dosage by intravenous infusion over a period of about 1-3 hours. In some embodiments, the compositions are provided in dosage forms suitable for continuous dosage by intravenous infusion over a period of about 1-2 hours. In some embodiments, the compositions are provided in dosage forms suitable for continuous dosage by intravenous infusion over a period of about 1 hour or less.

The percentage of a compound provided herein contained in such parenteral compositions is highly dependent on the specific nature thereof, as well as the activity of the compound and the needs of the subject. However, percentages of active ingredient of 0.01% to 10% in solution are employable and could be higher if the composition is a solid or suspension, which could be subsequently diluted to the above percentages. In some embodiments, the composition comprises about 0.1 to about 10% of the active agent in solution. In some embodiments, the composition comprises about 0.1 to about 5% of the active agent in solution. In some embodiments, the composition comprises about 0.1 to about 4% of the active agent in solution. In some embodiments, the composition comprises about 0.15 to about 3% of the active agent in solution. In some embodiments, the composition comprises about 0.2 to about 2% of the active agent in solution.

In some embodiments, these compositions are administered by intravenous infusion to humans at doses of about 5 mg/m² to about 300 mg/m². In some embodiments, these compositions are administered by intravenous infusion to humans at doses of about 5 mg/m² to about 200 mg/m². In some embodiments, these compositions are administered by intravenous infusion to humans at doses of about 5 mg/m² to about 100 mg/m². In some embodiments, these compositions are administered by intravenous infusion to humans at doses of about 10 mg/m² to about 50 mg/m². In some embodiments, these compositions are administered by intravenous infusion to humans at doses of about 50 mg/m² to about 200 mg/m². In some embodiments, these compositions are administered by intravenous infusion to humans at doses of about 75 mg/m² to about 175 mg/m². In some embodiments, these compositions are administered by intravenous infusion to humans at doses of about 100 mg/m² to about 150 mg/m².

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, the CLK inhibitor, DYRK inhibitor, dual CLK/DYRKA inhibitor, or a pharmaceutically acceptable salt or solvate of any of the foregoing disclosed herein can be formulated for respiratory delivery (either systemic or local), and can be administered as aqueous formulations, as non-aqueous solutions or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the CLK inhibitor, DYRK inhibitor, or dual CLK/DYRKA inhibitor that can be formulated for local or systemic respiratory delivery are compounds of Formulas (I)-(VIII), or a pharmaceutically acceptable salt or solvate thereof.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In some embodiments, the solid composition comprises multiple dosage units.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound. On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Also provided herein are kits that include one or more (e.g., 1, 2, 3, 4, 5, 6, 7, 8, 9, 10, 12, 14, 15, 16, 18, or 20) of any of the pharmaceutical compositions described herein that includes a therapeutically effective amount of any of the dual CLK/DYRKA inhibitors, or a pharmaceutically acceptable salt or solvate thereof, as described herein. Also provided herein are kits that include one or more (e.g., 1, 2, 3, 4, 5, 6, 7, 8, 9, 10, 12, 14, 15, 16, 18, or 20) of any of the pharmaceutical compositions described herein that includes a therapeutically effective amount of a combination of a CLK inhibitor, or a pharmaceutically acceptable salt or solvate thereof, and a DYRK inhibitor, or a pharmaceutically acceptable salt or solvate thereof. In some embodiments, the CLK inhibitor, DYRK inhibitor, or dual CLK/DYRKA inhibitor are independently selected from compounds of Formulas (I)-(VIII), described herein, or a pharmaceutically acceptable salt or solvate thereof.

In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with a cartilage disorder. In another embodiment, the kit can include a compound or composition as described herein and a label that indicates that the contents are to be administered to a patient with one or more of a particular cartilage disorders.

The kits described herein are not so limited; other variations will be apparent to one of ordinary skill in the art.

### EXAMPLES

The disclosure is further described in the following examples, which do not limit the scope of the disclosure described in the claims.

### General Procedures

Nuclear Speckles Assay

200,000 cells per well were plated on glass cover slips in 12-well plates and treated with the indicated concentrations of compound 123 (Table 3). After approximately 6 hrs, cells were fixed and stained with a phospho-SC35 antibody (Santa Cruz Biotechnology), followed by an Alexa-Fluor 488 secondary antibody (Thermo Fisher) and DAPI (Thermo Fisher). Cells were imaged at 100× magnification using the EVOS FL (Life Technologies, Carlsbad, Calif.).

Western Blots

Cells were plated at 300,000 cells per well in a 6-well plate and following an overnight incubation, treated with DMSO (vehicle control) or indicated compounds and incubated at 37° C. and 5% CO₂ for the indicated times. Following treatment, cells were trypsinized and pelleted by centrifugation and washed with PBS. Total protein from the cell pellet was extracted or for fractionation, protein was fractionated into cytoplasmic and nuclear fractions using a Nuclear and Cytoplasmic Extraction Reagents (NE-PER™) kit containing Halt™ protease and phosphatase inhibitors (Thermo Fisher). Protein concentrations were quantified using the Pierce Micro BCA protein assay kit (Thermo Fisher). 10-40 pg of reduced protein samples were resolved on NuPAGE 4-12% Bis-Tris gels and transferred onto nitrocellulose membranes (Thermo Fisher). Blots were blocked with non-fat dry milk. Primary antibodies were incubated overnight at 4° C. β-actin and Lamin B1 were used as loading controls (Supplementary Table S2 for primary antibodies). Mouse and rabbit horseradish peroxidase (HRP)-conjugated secondary antibodies were diluted in 5% blocking buffer in TBS-T. Protein-antibody complexes were detected by chemiluminescence using the SuperSignal West Femto Chemiluminescent Substrate (Thermo Fisher) and images were captured with a UVP ChemiDocIt2 camera system (Fisher Scientific, Hampton, N.H.).

qRT-PCR

Total RNA was isolated using a RNAeasy kit (Qiagen) as per the manufacturer's protocol. cDNA was synthesized using the iScript cDNA Synthesis Kit (Bio-Rad). Quantitative real-time PCR was performed using the CFX384 (Bio-Rad) using gene specific SYBR-green or TaqMan™ primers. Relative gene expression was determined by normalizing to housekeeping genes using the ΔΔCt method. Data was collected from at least 3 replicates per assay.

Nanostring Gene Expression Panel

Fifty nanograms of RNA was hybridized with Tagsets and probe pools from the nCounter® Vantage 3D™ Wnt Pathways Panel (NanoString Technologies) for 16 hrs at 67° C. Hybridized samples were ran on a nCounter® SPRINT Profiler (NanoString Technologies). Nanostring gene counts were normalized by the geometric mean of all housekeeping genes by nSolver (v3.0). P-values from normalized counts were calculated by an independent t-test and adjusted by the false discovery rate (FDR) method (Benjamini & Hochberg) to correct for multiple comparisons using R (v3.4.2). Data was plotted using R (v3.4.2).

siRNA Knockdowns

Reverse transfections were performed with siRNA (GE Dharmacon) control or a pool of hairpins targeting human CLK1, CLK2, CLK3, CLK4, SRSF5, SRSF6, DYRK1A mRNA using Lipofectamine RNAiMAX transfection reagent (ThermoFisher) (Supplementary Table 3 for list of siRNA) in serum- and antibiotic-free medium. 300,000 cells per well were plated in 6-well plates. Media was changed 24 hrs after transfection and cells were incubated for an additional 72 hrs at 37° C. and 5% CO2. Cells were collected by trypsinization and qRT-PCR, Nanostring gene expression, and Western blot analyses were performed as previously described.

In Vivo Animal Models

All animal housing and research procedures were performed at Samumed, LLC, 9381 Judicial Drive, Suite 160, San Diego, Calif. 92121, USA. The standards for animal husbandry and care followed were based on the U.S. Department of Agriculture's (USDA) Animal Welfare Act (9 CFR Parts 1, 2, and 3), the Guide for the Care and Use of Laboratory Animals, and approved Samumed, LLC Animal Committee protocols. The attending veterinarian was on-call during the live animal phase of this study.

Male Sprague Dawley (SD) rats (250 g±30 g) (Charles River, Wilmington, Mass.) were used for all animal studies. All animals were treatment naïve at the start of the studies. Procedures were performed during the hours of 8 am-5 pm on weekdays in a sterile hood in the vivarium. Rats were housed in individual ventilated cages (≤3 per cage) under the following conditions:

| | |
|---|---|
| Identification | Tail markings (lab marker) and cage cards |
| Acclimation | At least 48 hours |
| Caging | All animals were housed using Alternative Designs, MACS |
| | ventilated caging system. Quantity per cage: <3 |
| Environmental | Temperature was monitored and controlled between 68° F. and |
| Conditions | 73° F. Relative Humidity was controlled between 45% and 55%. |
| Photoperiod | 12-hour light/12-hour dark |
| Diet | Animals had ad libitum access to Certified Rodent Diet, Envigo |
| | T.2920X (irradiated). |
| Water | Animals had ad libitum access to RO water offered via 16 oz |
| | water bottles. |
| Bedding | All cages used Envigo Aspen Sani-chips bedding, T.7090A |
| | (¼″-½″ deep) |
| Sanitation | All cages were changed weekly, to include fresh bedding, feed, |
| | and water. Rooms and other equipment were sanitized weekly |
| | and/or more frequently if needed. |
| Welfare | Health Monitoring (HM) Plus with Helicobacter (Charles River) |
| assessments | tests were performed on sentinel rats for routine health |
| | surveillance. |
| | All study rats were monitored weekly by visual observation. No |
| | adverse events were observed and therefore no interventions |
| | were performed. |

Commonly used anesthetics such as isoflurane/O₂, disinfectants such as betadine or chlorhexidine solution, analgesics such as Buprenorphine (0.5 mg/kg) were used as necessary. All analysis where possible were performed blinded. Each treatment group was an experimental unit and no animals were excluded from any analysis.

Surgery-Induced OA Model (ACLT+pMMx)

For Nanostring analysis and qPCR: At 10 weeks postnatal age, 32 rats were subjected to severing of the anterior cruciate, medial collateral and medial meniscotibial ligaments (ACLT+pMMx). 8 rats (male, 10 weeks old) were untreated controls (Sham). Each rat was anesthetized with an isoflurane/O₂ mixture until the flexor withdrawal reflex was abolished. The surgeon wore a face mask, hair net, lab coat and shoe covers, and gloves that were disinfected with betadine or chlorhexidine. Surgery tools were sterilized with an autoclave or bead sterilizer and disinfected between animals using a bead sterilizer. After being shaved and disinfected with betadine or chlorhexidine solution, the right knee joint was exposed through a medial para-patellar approach. The medical collateral ligament (MCL) was transected with surgical scalpels. The patella was dislocated laterally, and the knee was placed in full flexion followed by anterior cruciate ligament (ACL) transection with surgical scalpels. The medial meniscus (MM) was also partially transected. After surgery, the joint surface was washed with sterile saline solution, and the capsule was sutured using Vicryl 4-0 (Ethicon, Edinburgh, UK) absorbable suture. The skin was closed with 9 mm auto clips (Mikron precision INC, Gardena Calif.). Buprenorphine (0.5 mg/kg) was administered immediately after the surgery and daily for one-week post-surgery. All the surgery, rats were allowed to move freely in plastic cages until their necropsies. One-week post-surgery, rats were randomized into each group by picking numbers from the envelop and given IA compound 123 (0.1 μg, 0.3 μg, 1 μg in 50 μL) or vehicle (n=8 rats per group). At the indicated timepoints, animals were sacrificed using Carbon dioxide (CO₂). Cartilage was isolated, flash frozen in liquid Nitrogen and stored at −80° C. until processing for RNA extraction.

For Western blot: At 10 weeks postnatal age, 16 rats were subjected to severing of the anterior cruciate, medial collateral and medial meniscotibial ligaments (ACLT+pMMx), randomized and treated with compound 123 or vehicle (n=4 rats per group), as described above. At the indicated timepoints, animals were sacrificed using Carbon dioxide (CO₂). Cartilage was isolated, flash frozen in liquid Nitrogen and stored at −80° C. until processing for protein extraction.

Monosodium Iodoacetate (MIA) Injection Induced OA Model

For cytokine, MMP, histology (H&E and Safranin O-Fast Green staining), OARSI scores and weight bearing measurements: At 10 weeks postnatal age, 20 rats were subjected to MIA injection (3 mg in 50 μL). Each rat was anesthetized with an isoflurane/O₂ mixture until the flexor withdrawal reflex was abolished. The surgeon wore a face mask, hair net, lab coat and shoe covers, and gloves that were disinfected with betadine or chlorhexidine between animals. All tools were sterilized with an autoclave. MIA was dissolved in 0.9% (w/v) saline (3 mg in 50 μL). The solution was filtrated with 0.22 μm membrane before administration. After being shaved and disinfected with betadine or chlorhexidine solution, the right knees were injected intra-articularly with 50 μL of MIA using 0.5-in 26-gauge needle, while the contralateral knees received an IA injection of 0.9% saline. After injection, the injected surface was washed with sterile saline solution. All the rats were allowed to move freely in plastic cages until their necropsies at different time points post-MIA injection.

10 rats (male, 10 weeks old) were untreated controls. MIA injected rats were randomized into each group by picking numbers from the envelop and were given IA compound 123 (0.3 μg in 50 μL) or vehicle (n=10 rats per group). At time points of day 1, 11, 28, knee joints were isolated for biochemical analysis or histology. For biochemical analysis, knee joints were frozen in liquid nitrogen.

For Western blot: At 10 weeks postnatal age, 20 rats were subjected to MIA injection (3 mg in 50 μL), randomized and treated with compound 123 (0.1 μg, 0.3 μg, 1.0 μg) or vehicle (n=5 rats per group), as described above. On day 11, animals were sacrificed using Carbon dioxide (CO₂). Cartilage was isolated, flash frozen in liquid Nitrogen and stored at −80° C. until processing for protein extraction.

Primary and Secondary Antibodies Used

| Antibody Against | Source | Catalog |
|---|---|---|
| CLK1 | Abcam | ab74044 |
| CLK2 | Abcam | ab65082 |
| CLK3 | Cell Signaling Technology | 3256 |
| CLK4 | Abcam | ab67936 |
| DYRK1A | Cell Signaling Technology | 8765 |
| HIPK2 | Cell Signaling Technology | 5091 |
| Anti-Phosphoepitope SR proteins | EMD Millipore | MABE50 |
| SRSF1 | Abcam | ab38017 |
| SRSF5 | Sigma Aldrich | HPA043484 |
| SRSF6 | LifeSpan BioSciences, Inc. | LS-C290327 |
| Phospho-NF-κB p65 (Ser468) | Cell Signaling Technology | 3039 |
| total NFkB p65 | Cell Signaling Technology | 8242S |
| Phospho-NF-κB p105 (Ser933) (18E6) | Cell Signaling Technology | 4806 |
| NF-κB1 p105/p50 (D4P4D) | Cell Signaling Technology | 13586 |
| phospho-cJun | Cell Signaling Technology | 2361S |
| Phospho-FoxO1 (Thr24)/FoxO3a (Thr32) | Cell Signaling Technology | 9464 |
| FOXO1 (C29H4) | Cell Signaling Technology | 2880 |
| Phospho-SAPK/JNK (Thr183/Tyr185) | Cell Signaling Technology | 9251 |
| SAPK/JNK Antibody | Cell Signaling Technology | 9252 |
| Phospho-SirT1 (Ser27) | Cell Signaling Technology | 2327 |
| Phospho-SirT1 (Ser47) | Cell Signaling Technology | 2314 |
| SirT1 Antibody | Cell Signaling Technology | 2310 |
| Phospho-Stat3 (Ser727) | Cell Signaling Technology | 9134 |
| Phospho-Stat3 (Tyr705) (D3A7) | Cell Signaling Technology | 9145 |
| Stat3 | Cell Signaling Technology | 9139 |
| Pp38/MAPK | Cell Signaling Technology | 9211S |
| Total p38/MAPk | Cell Signaling Technology | 9212S |
| GAPDH | Cell Signaling Technology | 8884 |
| Lamin B1 (D9V6H) | Cell Signaling Technology | 15068 |
| TATA binding protein (TBP) | Abcam | ab63766 |
| β-Actin (13E5) | Cell Signaling Technology | 5125 |
| β-Catenin (D10A8) | Cell Signaling Technology | 8480 |
| TCF7 | Cell Signaling Technology | 2203 |
| TCF4 | Cell Signaling Technology | 2569 |
| LEF1 | Cell Signaling Technology | 2230 |
| AXIN2 | Abcam | ab32197 |
| AXIN2 | Cell Signaling Technology | 2151 |
| AML1 (D33G6) (RUNX1) | Cell Signaling Technology | 4336 |
| COMP | R&D Systems | AF3134 |
| SOX9 | Abcam | ab85230 |
| TLR4 | Abcam | abl3867 |
| phospho-AKT | Cell Signaling Technology | 9271S |

siRNAs Used

| Gene | siRNA | Source | Catalog Number |
|---|---|---|---|
| Control | ON-TARGETplus Non-targeting Control Pool | Dharmacon | D-001810-10 |
| CLK1 | Individual: ON-TARGETplus CLK1 siRNA Targeted | Dharmacon | J-004800-07 |
| | Region: Non-Coding,ORF | | |
| CLK2 | MISSION ® siRNA Human Kinase CLK2 (siRNA6) | Sigma | SIHK0460 |
| | | Aldrich | |
| CLK2 | Individual: ON-TARGETplus CLK2 siRNA Targeted | Dharmacon | J-004801-10 |
| | Region: 5′UTR,Non-Coding,ORF | | |
| CLK3 | Individual: ON-TARGETplus CLK3 siRNA Targeted | Dharmacon | J-004802-09 |
| | Region: Non-Coding,ORF | | |
| CLK4 | SMARTpool: ON-TARGETplus CLK4 siRNA | Dharmacon | L-004803-00 |
| DYRK1A | SMARTpool: ON-TARGETplus DYRK1A siRNA | Dharmacon | L-004805-00 |
| CTNNB1 | SMARTpool: siGENOME CTNNB1 siRNA | Dharmacon | M-003482-00 |
| TCF7 | SMARTpool: ON-TARGETplus Tcf7 siRNA | Dharmacon | L-019735-00 |
| SRSF1 | SMARTpool: ON-TARGETplus SRSF1 siRNA | Dharmacon | L-018672-01 |
| SRSF4 | SMARTpool: ON-TARGETplus SRSF4 siRNA | Dharmacon | L-005151-01 |
| SRSF5 | SMARTpool: ON-TARGETplus SRSF5 | Dharmacon | L-007279-01 |
| SRSF6 | SMARTpool: ON-TARGETplus SRSF6 siRNA | Dharmacon | L-016067-01 |
| STAT3 | SMARTpool: ON-TARGETplus STAT3 siRNA | Dharmacon | L-003544-00 |
| RELB | SMARTpool: ON-TARGETplus RELB | Dharmacon | L-004767-00 |
| RELA | SMARTpool: ON-TARGETplus RELA | Dharmacon | L-003533-00 |
| HIPK2 | SMARTpool: ON-TARGETplus HIPK2 siRNA | Dharmacon | L-003266-00 |
| TCF4 | SMARTpool: ON-TARGETplus TCF7L2 siRNA | Dharmacon | L-003816-00 |
| LEF1 | SMARTpool: ON-TARGETplus LEF1 siRNA | Dharmacon | L-015396-00 |

### Example 1. Wnt Pathway Activity Screening Assay

The screening assay for Wnt pathway activity is described as follows. Reporter cell lines can be generated by stably transducing cancer cell lines (e.g., colon cancer) or primary cells (e.g., IEC-6 intestinal cells) with a lentiviral construct that includes a Wnt-responsive promoter driving expression of the firefly luciferase gene.

SW480 colon carcinoma cells were transduced with a lentiviral vector expressing luciferase with a human Sp5 promoter consisting of a sequence of eight TCF/LEF binding sites. SW480 cells stably expressing the Sp5-Luc reporter gene and a hygromycin resistance gene were selected by treatment with 150 μg/mL of hygromycin for 7 days. These stably transduced SW480 cells were expanded in cell culture and used for all further screening activities. Each compound was dissolved in DMSO as a 10 mM stock and used to prepare compound source plates. Serial dilution (1:3, 10-point dose-response curves starting from 10 μM) and compound transfer was performed using the ECHO 550 (Labcyte, Sunnyvale, Calif.) into 384-well white solid bottom assay plates (Greiner Bio-One) with appropriate DMSO backfill for a final DMSO concentration of 0.1%. For Sp5-Luc reporter gene assays, the cells were plated at 4,000 cells/well in 384-well plates with a DMEM medium containing 1% fetal bovine serum, and 1% Penicillin-Streptomycin and incubated for 36 to 48 hours at 37° C. and 5% CO₂. Following incubation, 15 μl of BriteLite Plus luminescence reagent (Perkin Elmer) was added to each well of the 384-well assay plates. The plates were placed on an orbital shaker for 2 min and then luminescence was quantified using the Envision (Perkin Elmer) plate reader. Readings were normalized to DMSO only treated cells, and normalized activities were utilized for EC₅₀ calculations using the dose-response log (inhibitor) vs. response-variable slope (four parameters) nonlinear regression feature available in GraphPad Prism 5.0 (or Dotmatics). For EC₅₀ of >10 μM, the percent inhibition at 10 μM is provided.

Table 4 shows the measured activity for representative compounds of Formulas (I)-(VIII) as described herein.

TABLE 4

| Compound | EC₅₀ (μM) |
|---|---|
| 1 | 0.0205 |
| 2 | 0.4790 |
| 3 | 0.1187 |
| 4 | 0.1229 |
| 5 | 0.0223 |
| 6 | 0.0057 |
| 7 | 0.6809 |
| 8 | 0.6132 |
| 9 | 0.5581 |
| 10 | 0.0903 |
| 11 | 0.0025 |
| 12 | 2.2197 |
| 13 | 0.6619 |
| 14 | 1.5970 |
| 15 | 0.0029 |
| 16 | 0.6790 |
| 17 | 0.9877 |
| 18 | 2.1485 |
| 19 | 1.8500 |
| 20 | 1.6480 |
| 21 | 0.9642 |
| 22 | >10 |
| 23 | 1.5059 |
| 24 | 1.4515 |
| 25 | 0.3250 |
| 26 | 0.0821 |
| 27 | >10 |
| 28 | 0.4089 |
| 29 | >10 |
| 30 | 3.5744 |
| 31 | 1.2060 |
| 32 | 1.2680 |
| 33 | 4.6397 |
| 34 | 1.6370 |
| 35 | 0.2615 |
| 36 | 3.5070 |
| 37 | 1.7320 |
| 38 | 1.4280 |
| 39 | 1.8980 |
| 40 | 2.4370 |
| 41 | 0.2562 |
| 42 | 0.0843 |
| 43 | 0.0950 |
| 44 | 0.6270 |
| 45 | 0.8339 |
| 46 | 0.6300 |
| 47 | 0.2266 |
| 48 | 0.7800 |
| 49 | 3.2460 |
| 50 | 2.8060 |
| 51 | 0.8088 |
| 52 | 0.2526 |
| 53 | 1.1517 |
| 54 | 0.1760 |
| 55 | 0.0800 |
| 56 | 1.2500 |
| 57 | 0.2260 |
| 58 | 0.4637 |
| 59 | 1.3942 |
| 60 | 1.0905 |
| 61 | 0.7950 |
| 62 | 0.0370 |
| 63 | 0.0680 |
| 64 | 0.1060 |
| 65 | 0.1963 |
| 66 | 0.4770 |
| 67 | 0.0966 |
| 68 | >10 |
| 69 | >10 |
| 70 | 0.3300 |
| 71 | 0.0500 |
| 72 | 0.0050 |
| 73 | 0.0040 |
| 74 | 1.9600 |
| 75 | 0.0471 |
| 76 | 1.0327 |
| 77 | 1.8160 |
| 78 | 0.0570 |
| 79 | 0.0590 |
| 80 | 0.0364 |
| 81 | 0.0150 |
| 82 | 0.0410 |
| 83 | 0.5000 |
| 84 | 0.0650 |
| 85 | 0.0250 |
| 86 | 0.1200 |
| 87 | 0.0130 |
| 88 | 0.0300 |
| 89 | 0.0270 |
| 90 | 1.5000 |
| 91 | 0.0490 |
| 92 | 0.0028 |
| 93 | 0.1010 |
| 94 | 0.0400 |
| 95 | 0.2890 |
| 96 | 0.0300 |
| 97 | 0.1250 |
| 98 | 0.0070 |
| 99 | 0.4300 |
| 100 | 0.2100 |
| 101 | 0.0670 |
| 102 | 0.0610 |
| 103 | 1.3066 |
| 104 | 1.6570 |
| 105 | 2.3990 |
| 106 | 0.0790 |
| 107 | 0.2420 |
| 108 | 0.3148 |
| 109 | 0.2300 |
| 110 | 0.1205 |
| 111 | 1.1493 |
| 112 | 0.7930 |
| 113 | 0.4514 |
| 114 | 0.0950 |
| 115 | 0.3350 |
| 116 | 0.6840 |
| 117 | 0.6451 |
| 118 | 0.8485 |
| 119 | 0.0060 |
| 120 | 0.0046 |
| 121 | >10 |
| 122 | 0.0188 |
| 123 | 0.0034 |
| 124 | 0.0014 |
| 125 | 0.0006 |
| 126 | 0.0110 |
| 127 | 0.0090 |
| 128 | 0.3450 |
| 129 | 0.0230 |
| 130 | 0.0100 |
| 131 | 0.0012 |
| 132 | 0.6380 |
| 133 | 0.6586 |
| 134 | 0.0409 |
| 135 | 0.0822 |
| 136 | 0.6373 |
| 137 | 0.0130 |
| 138 | 1.0548 |
| 139 | 1.0473 |
| 140 | 0.6427 |
| 141 | 0.0264 |
| 142 | 0.0191 |
| 143 | 0.0110 |
| 144 | 0.4600 |
| 145 | >10 |
| 146 | 0.3750 |
| 147 | 0.0313 |
| 148 | 0.1200 |
| 149 | 0.2400 |
| 150 | 0.2400 |
| 151 | 0.2529 |
| 152 | >10 |
| 153 | 0.0302 |
| 154 | >10 |
| 155 | 0.0111 |
| 156 | 0.0100 |
| 157 | 0.0700 |
| 158 | 0.1500 |
| 159 | 0.0125 |
| 160 | 0.0110 |
| 161 | 0.0040 |
| 162 | 2.7262 |
| 163 | 0.0570 |
| 164 | 0.0057 |
| 165 | 0.0088 |
| 166 | 1.0701 |
| 167 | 0.0020 |
| 168 | 0.0123 |
| 169 | >10 |
| 170 | >10 |
| 171 | >10 |
| 172 | 0.0080 |
| 173 | 0.0040 |
| 174 | >10 |
| 175 | 0.8230 |
| 176 | >10 |
| 177 | 0.0010 |
| 178 | 0.0350 |
| 179 | 0.0924 |
| 180 | 1.1205 |
| 181 | >10 |
| 182 | 0.0283 |
| 183 | >10 |
| 184 | >10 |
| 185 | 1.5068 |
| 186 | 0.0051 |
| 187 | >10 |
| 188 | 0.0854 |
| 189 | 0.0120 |
| 190 | 0.0233 |
| 191 | 0.0354 |
| 192 | >10 |
| 193 | 0.9810 |
| 194 | 1.2719 |
| 195 | 1.7510 |
| 196 | 0.9990 |
| 198 | 0.9990 |
| 199 | 2.1258 |
| 200 | 0.0201 |
| 201 | >10 |
| 202 | 0.0210 |
| 203 | >10 |
| 204 | 0.0080 |
| 205 | 0.3440 |
| 206 | >10 |
| 207 | >10 |
| 208 | 0.0420 |
| 209 | 0.0410 |
| 210 | 0.0390 |
| 211 | 0.0200 |
| 212 | 0.1163 |
| 213 | 0.0530 |
| 214 | >10 |
| 215 | >10 |
| 216 | >10 |
| 217 | 0.2681 |
| 218 | 0.0830 |
| 219 | 0.0857 |
| 220 | 0.0230 |
| 221 | >10 |
| 222 | >10 |
| 223 | >10 |
| 224 | 0.0104 |
| 225 | 4.5673 |
| 226 | 2.8972 |
| 227 | >10 |
| 228 | >10 |
| 229 | 0.0454 |
| 230 | 0.0630 |
| 231 | >10 |
| 232 | 0.0700 |
| 233 | 0.0553 |
| 234 | 0.0330 |
| 235 | 0.0510 |
| 236 | 0.0435 |
| 237 | 0.0526 |
| 238 | 0.2972 |
| 239 | 0.5969 |
| 240 | 0.2981 |
| 241 | 0.1621 |
| 242 | 0.0189 |
| 243 | 0.0100 |
| 244 | 0.0020 |
| 245 | 0.0077 |
| 246 | 0.0615 |
| 247 | 3.6404 |
| 248 | >10 |
| 249 | 0.0593 |
| 250 | 0.0580 |
| 251 | 0.0320 |
| 252 | 0.0570 |
| 253 | 0.0018 |
| 254 | 0.0120 |
| 255 | 0.0040 |
| 256 | 0.0001 |
| 257 | 0.0222 |
| 258 | 0.7095 |
| 259 | 0.0980 |
| 260 | 0.0480 |
| 261 | 3.1130 |
| 262 | 0.0167 |
| 263 | 0.0353 |
| 264 | 0.4700 |
| 265 | 0.5512 |
| 266 | 1.4820 |
| 267 | 0.8229 |
| 268 | 0.8002 |
| 269 | 0.3544 |
| 270 | 3.3289 |
| 271 | 0.0663 |
| 272 | 0.0019 |
| 273 | 0.0092 |
| 274 | 0.0109 |
| 275 | 0.0455 |
| 276 | 0.0407 |
| 277 | 0.0101 |
| 278 | 0.0210 |
| 279 | >10 |
| 280 | >10 |
| 281 | 0.0220 |
| 282 | 0.2019 |
| 283 | 0.1544 |
| 284 | 1.6613 |
| 285 | >10 |
| 286 | >10 |
| 287 | 0.6199 |
| 288 | 0.0304 |
| 289 | 0.1244 |
| 290 | 0.0966 |
| 291 | 0.1092 |
| 292 | 0.0050 |
| 293 | 0.0405 |
| 294 | 0.0210 |
| 295 | 0.3774 |
| 296 | >10 |
| 297 | 1.6066 |
| 298 | >10 |
| 299 | 0.0183 |
| 300 | 0.5725 |
| 301 | 1.3544 |
| 302 | 0.0450 |
| 303 | 0.0450 |
| 304 | 0.0132 |
| 305 | 0.2464 |
| 306 | 0.3050 |
| 307 | 1.7347 |
| 308 | 2.9450 |
| 309 | 1.2850 |
| 310 | 1.1759 |
| 311 | 1.8500 |
| 312 | 3.7479 |
| 313 | 3.1153 |
| 314 | 0.8438 |
| 315 | 3.6150 |
| 316 | 0.2912 |
| 317 | 3.2588 |
| 318 | 3.0035 |
| 319 | 0.5937 |
| 320 | 0.1314 |
| 321 | 0.1974 |
| 322 | 0.7452 |
| 323 | 0.2240 |
| 324 | 1.6304 |
| 325 | >10 |
| 326 | 0.6047 |
| 327 | 1.0300 |
| 328 | 0.3206 |
| 329 | 0.3903 |
| 330 | 0.6416 |
| 331 | 0.4180 |
| 332 | 0.4349 |
| 333 | >10 |
| 334 | 2.2950 |
| 335 | >10 |
| 336 | 2.4870 |
| 337 | 1.3682 |
| 338 | 1.9849 |
| 339 | 0.3746 |
| 340 | 0.0305 |
| 341 | 0.3100 |
| 342 | >10 |
| 343 | 1.3450 |
| 344 | >10 |
| 345 | 2.2496 |
| 346 | >10 |
| 347 | 0.1463 |
| 348 | 0.1802 |
| 349 | 0.2600 |
| 350 | 0.2750 |
| 351 | >10 |
| 352 | >10 |
| 353 | >10 |
| 354 | 0.3302 |
| 355 | 0.2017 |
| 356 | 1.3537 |
| 357 | 0.4214 |
| 358 | >10 |
| 359 | 0.1000 |
| 360 | 0.6700 |
| 361 | >10 |
| 362 | >10 |
| 363 | >10 |
| 364 | 0.2549 |
| 365 | >10 |
| 366 | >10 |
| 367 | 3.7284 |
| 368 | 0.4151 |
| 369 | >10 |
| 370 | 0.5226 |
| 371 | 3.8539 |
| 372 | >10 |
| 373 | >10 |
| 374 | >10 |
| 375 | 0.2500 |
| 376 | >10 |
| 377 | 0.6579 |
| 378 | >10 |
| 379 | >10 |
| 380 | >10 |
| 381 | >10 |
| 382 | >10 |
| 383 | >10 |
| 384 | 0.9990 |
| 385 | >10 |
| 386 | >10 |
| 387 | >10 |
| 388 | 0.8750 |
| 389 | 0.7106 |
| 390 | >10 |
| 391 | >10 |
| 392 | >10 |
| 393 | >10 |
| 394 | >10 |
| 395 | >10 |
| 396 | >10 |
| 397 | >10 |
| 398 | 1.0550 |
| 399 | >10 |
| 400 | >10 |
| 401 | 0.7078 |
| 402 | 1.5068 |
| 403 | >10 |
| 404 | >10 |
| 405 | >10 |
| 406 | >10 |
| 407 | 0.0224 |
| 408 | 1.4390 |
| 409 | 0.4322 |
| 410 | 0.0400 |
| 411 | 1.0650 |
| 412 | 0.1330 |
| 413 | >10 |
| 414 | >10 |
| 415 | 0.1911 |
| 416 | 0.3887 |
| 417 | 0.1017 |
| 418 | 0.9600 |
| 419 | 0.3986 |
| 420 | 0.3240 |
| 421 | 1.4690 |
| 422 | 3.5740 |
| 423 | >10 |
| 424 | 0.6050 |
| 425 | 1.8900 |
| 426 | 1.5362 |
| 427 | >10 |
| 428 | 0.0750 |
| 429 | 0.2710 |
| 430 | 2.6291 |
| 431 | 0.2850 |
| 432 | 1.2600 |
| 433 | 1.6250 |
| 434 | 1.7550 |
| 435 | >10 |
| 436 | >10 |
| 437 | 0.5800 |
| 438 | 0.3950 |
| 439 | 1.0350 |
| 440 | 3.7584 |
| 441 | 3.0583 |
| 442 | 1.1353 |
| 443 | >10 |
| 444 | 1.4123 |
| 445 | 1.3496 |
| 446 | 0.0450 |
| 447 | 0.1037 |
| 448 | 0.6850 |
| 449 | 0.1651 |
| 450 | 1.6714 |
| 451 | 1.5407 |
| 452 | 1.8451 |
| 453 | 3.2950 |
| 454 | >10 |
| 455 | 0.6039 |
| 456 | 2.9580 |
| 457 | 0.4450 |
| 458 | 0.5723 |
| 459 | 4.0486 |
| 460 | 3.8519 |
| 461 | 4.0743 |
| 462 | >10 |
| 463 | 3.4100 |
| 464 | 0.5750 |
| 465 | 4.7090 |
| 466 | 1.8375 |
| 467 | 0.7500 |
| 468 | 5.2741 |
| 469 | 4.2100 |
| 470 | 8.2523 |
| 471 | 1.1716 |
| 472 | >10 |
| 473 | >10 |
| 474 | 0.7000 |
| 475 | 7.6900 |
| 476 | >10 |
| 477 | 3.4472 |
| 478 | 1.8800 |
| 479 | 3.4771 |
| 480 | 3.8654 |
| 481 | >10 |
| 482 | 5.2700 |
| 483 | 3.4243 |
| 484 | >10 |
| 485 | >10 |
| 486 | 2.1350 |
| 487 | 2.8033 |
| 488 | 6.0953 |
| 489 | >10 |
| 490 | 3.2916 |
| 491 | 3.8928 |
| 492 | 9.3750 |
| 493 | 4.9690 |
| 494 | 4.7500 |
| 494 | >10 |
| 496 | 4.1638 |
| 497 | >10 |
| 498 | >10 |
| 499 | 0.9150 |
| 500 | 0.8900 |
| 501 | >10 |
| 502 | 3.6415 |
| 503 | 1.0041 |
| 504 | 0.6429 |
| 505 | 1.9107 |
| 506 | 0.5723 |
| 507 | 0.4733 |
| 508 | 0.5780 |
| 509 | 3.6848 |
| 510 | 3.5514 |
| 511 | 0.4073 |
| 512 | >10 |
| 513 | 0.3864 |
| 514 | 1.2732 |
| 515 | 0.4051 |
| 516 | 0.4197 |
| 517 | 0.2441 |
| 518 | 1.6829 |
| 519 | >10 |
| 520 | >10 |
| 521 | >10 |
| 522 | 1.5135 |
| 523 | 2.5118 |
| 524 | >10 |
| 525 | 0.4787 |
| 526 | 1.3039 |
| 527 | 0.5536 |
| 528 | 3.6395 |
| 529 | >10 |
| 530 | >10 |
| 531 | >10 |
| 532 | >10 |
| 533 | >10 |
| 534 | >10 |
| 535 | 1.8531 |
| 536 | 1.1044 |
| 537 | 3.9923 |
| 538 | 1.0263 |
| 539 | 0.5134 |
| 540 | 0.0439 |
| 541 | 0.1038 |
| 542 | 0.1530 |
| 543 | 0.1275 |
| 544 | 0.1084 |
| 545 | 0.0215 |
| 546 | 0.0585 |
| 547 | 2.1238 |
| 548 | 0.1309 |
| 549 | 0.1288 |
| 550 | 0.0935 |
| 551 | 0.1493 |
| 552 | 0.0182 |
| 553 | 0.1615 |
| 554 | 0.0363 |
| 555 | 0.0656 |
| 556 | 0.4223 |
| 557 | 0.1839 |
| 558 | 0.1439 |
| 559 | 0.2498 |
| 560 | 0.3652 |
| 561 | 0.0140 |
| 562 | 0.0545 |
| 563 | 0.0800 |
| 564 | 1.3194 |
| 565 | 0.4248 |
| 566 | 0.0466 |
| 567 | 0.2646 |
| 568 | 0.2695 |
| 569 | 0.4709 |
| 570 | 0.5935 |
| 571 | 0.7057 |
| 572 | 0.1892 |
| 573 | 0.0230 |
| 574 | 0.3751 |
| 575 | 0.1611 |
| 576 | 0.1885 |
| 577 | 0.2560 |
| 578 | 0.1168 |
| 579 | 0.0327 |
| 580 | 0.4717 |
| 581 | 0.0569 |
| 582 | 0.0926 |
| 583 | 0.1074 |
| 584 | 1.8905 |
| 585 | 0.0348 |
| 586 | 0.1146 |
| 587 | 2.2058 |
| 588 | 0.5724 |
| 589 | 0.1017 |
| 590 | 1.0194 |
| 591 | 0.0570 |
| 592 | 0.3463 |
| 593 | 0.0131 |
| 594 | 0.0409 |
| 595 | 0.0686 |
| 596 | 0.7096 |
| 597 | 0.3819 |
| 598 | 0.1779 |
| 599 | 0.1323 |
| 600 | 0.3809 |
| 601 | 0.3193 |
| 602 | 0.1186 |
| 603 | 0.0095 |
| 604 | 0.0133 |
| 605 | 0.0268 |
| 606 | 0.1015 |
| 607 | 0.4678 |
| 608 | 0.4469 |
| 609 | 0.8240 |
| 610 | 0.6067 |
| 611 | 0.1070 |
| 612 | 0.3571 |
| 613 | 3.6364 |
| 614 | 0.1645 |
| 615 | 0.3828 |
| 616 | 1.4015 |
| 617 | 0.5774 |
| 618 | 0.0418 |
| 619 | 0.0518 |
| 620 | 0.0868 |
| 621 | 0.3380 |
| 622 | >10 |
| 623 | 0.2845 |
| 624 | 0.0733 |
| 625 | >10 |
| 626 | >10 |
| 627 | >10 |
| 628 | >10 |
| 629 | 1.080 |
| 630 | >10 |
| 631 | 3.381 |
| 632 | >10 |
| 633 | >10 |
| 634 | 3.662 |
| 635 | 6.071 |
| 636 | 0.097 |
| 637 | >10 |
| 638 | >10 |
| 639 | >10 |
| 640 | >10 |
| 641 | >10 |
| 642 | >10 |

### Example 2. DYRK1A Kinase Activity Assay

Representative compounds were screened using the assay procedure for DYRK1A kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, Calif.) into 1536-well black-walled round bottom plates (Corning).

The DYRK1A kinase assay was run using the Ser/Thr 18 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant DYRK1A kinase, ATP and Ser/Thr peptide 18 were prepared in 1× Kinase buffer to final concentrations of 0.19 μg/mL, 30 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (1 μM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio×F100%)−C100%)/((C0%−C100%)+(Em ratio×(F100%−F0%)))]. Dose-response curves were generated and inhibitory concentration (IC₅₀) values were calculated using non-linear regression curve fit in the Dotmatics' Studies Software (Bishops Stortford, UK).

Table 5 shows the measured activity for representative compounds of Formulas (I)-(VII) as described herein.

TABLE 5

| Compound | EC₅₀ (μM) |
|---|---|
| 1 | 0.012 |
| 2 | 0.063 |
| 3 | 0.027 |
| 4 | 0.010 |
| 5 | 0.012 |
| 6 | 0.014 |
| 7 | 0.007 |
| 8 | 0.008 |
| 9 | 0.008 |
| 10 | 0.004 |
| 11 | 0.009 |
| 12 | 0.010 |
| 13 | 0.010 |
| 14 | 0.012 |
| 15 | 0.004 |
| 16 | 0.022 |
| 17 | 0.004 |
| 18 | 0.033 |
| 19 | 0.004 |
| 20 | 0.001 |
| 21 | 0.001 |
| 22 | 0.020 |
| 23 | 0.015 |
| 24 | 0.005 |
| 25 | 0.010 |
| 26 | 0.020 |
| 27 | 0.021 |
| 28 | 0.024 |
| 29 | 0.011 |
| 30 | 0.009 |
| 31 | 0.019 |
| 32 | 0.024 |
| 33 | 0.056 |
| 34 | 0.021 |
| 35 | 0.002 |
| 36 | 0.063 |
| 37 | 0.054 |
| 38 | 0.050 |
| 39 | 0.022 |
| 40 | 0.060 |
| 41 | 0.009 |
| 42 | 0.001 |
| 43 | 0.001 |
| 44 | 0.011 |
| 45 | 0.021 |
| 46 | 0.006 |
| 47 | 0.002 |
| 48 | 0.011 |
| 49 | 0.025 |
| 50 | 0.056 |
| 51 | 0.045 |
| 52 | 0.004 |
| 53 | 0.032 |
| 54 | 0.001 |
| 55 | 0.001 |
| 56 | 0.002 |
| 57 | 0.003 |
| 58 | 0.005 |
| 59 | 0.004 |
| 60 | 0.017 |
| 61 | 0.006 |
| 62 | 0.001 |
| 63 | 0.006 |
| 64 | 0.001 |
| 65 | 0.001 |
| 66 | 0.004 |
| 67 | 0.001 |
| 68 | 0.003 |
| 69 | 0.002 |
| 70 | 0.002 |
| 71 | 0.002 |
| 72 | 0.002 |
| 73 | 0.001 |
| 74 | 0.006 |
| 75 | 0.006 |
| 76 | 0.001 |
| 77 | 0.002 |
| 78 | 0.001 |
| 79 | 0.002 |
| 80 | 0.004 |
| 81 | 0.001 |
| 82 | 0.017 |
| 83 | 0.004 |
| 84 | 0.001 |
| 85 | 0.059 |
| 86 | 0.038 |
| 87 | 0.001 |
| 88 | 0.001 |
| 89 | 0.001 |
| 90 | 0.002 |
| 91 | 0.001 |
| 92 | 0.001 |
| 93 | 0.007 |
| 94 | 0.001 |
| 95 | 0.001 |
| 96 | 0.001 |
| 97 | 0.003 |
| 98 | 0.002 |
| 99 | 0.002 |
| 100 | 0.002 |
| 101 | 0.001 |
| 102 | 0.004 |
| 103 | 0.004 |
| 104 | 0.004 |
| 105 | 0.001 |
| 106 | 0.001 |
| 107 | 0.001 |
| 108 | 0.001 |
| 109 | 0.002 |
| 110 | 0.001 |
| 111 | 0.001 |
| 112 | 0.002 |
| 113 | 0.004 |
| 114 | 0.003 |
| 115 | 0.004 |
| 116 | 0.016 |
| 117 | 0.003 |
| 118 | 0.004 |
| 119 | 0.012 |
| 120 | 0.061 |
| 121 | 0.036 |
| 122 | 0.053 |
| 123 | 0.005 |
| 124 | 0.043 |
| 125 | 0.002 |
| 126 | 0.075 |
| 127 | 0.014 |
| 128 | 0.005 |
| 129 | 0.024 |
| 130 | 0.004 |
| 131 | 0.020 |
| 132 | 0.025 |
| 133 | 0.025 |
| 134 | 0.048 |
| 135 | 0.015 |
| 136 | 0.065 |
| 137 | 0.005 |
| 138 | 0.048 |
| 139 | 0.062 |
| 140 | 0.008 |
| 141 | 0.028 |
| 142 | 0.080 |
| 143 | 0.012 |
| 144 | 0.024 |
| 145 | 0.031 |
| 146 | 0.043 |
| 147 | 0.026 |
| 148 | 0.046 |
| 149 | 0.040 |
| 150 | 0.051 |
| 151 | 0.005 |
| 152 | 0.005 |
| 153 | 0.016 |
| 154 | 0.016 |
| 155 | 0.039 |
| 156 | 0.026 |
| 157 | 0.015 |
| 158 | 0.080 |
| 159 | 0.005 |
| 160 | 0.011 |
| 161 | 0.055 |
| 162 | 0.064 |
| 163 | 0.060 |
| 164 | 0.073 |
| 165 | 0.006 |
| 166 | 0.040 |
| 167 | 0.005 |
| 168 | 0.050 |
| 169 | 0.008 |
| 170 | 0.007 |
| 171 | 0.015 |
| 172 | 0.070 |
| 173 | 0.010 |
| 174 | 0.030 |
| 175 | 0.030 |
| 176 | 0.086 |
| 177 | 0.036 |
| 178 | 0.076 |
| 179 | 0.042 |
| 180 | 0.053 |
| 181 | 0.086 |
| 182 | 0.029 |
| 183 | 0.036 |
| 184 | 0.047 |
| 185 | 0.028 |
| 186 | 0.010 |
| 187 | 0.030 |
| 188 | 0.005 |
| 189 | 0.012 |
| 190 | 0.014 |
| 191 | 0.011 |
| 192 | 0.006 |
| 193 | 0.014 |
| 194 | 0.015 |
| 195 | 0.014 |
| 196 | 0.013 |
| 198 | 0.026 |
| 199 | 0.010 |
| 200 | 0.072 |
| 201 | 0.097 |
| 202 | 0.003 |
| 203 | 0.021 |
| 204 | 0.042 |
| 205 | 0.003 |
| 206 | 0.016 |
| 207 | 0.090 |
| 208 | 0.014 |
| 209 | 0.008 |
| 210 | 0.004 |
| 211 | 0.022 |
| 212 | 0.006 |
| 213 | 0.002 |
| 214 | 0.045 |
| 215 | 0.009 |
| 216 | 0.093 |
| 217 | 0.005 |
| 218 | 0.002 |
| 219 | 0.004 |
| 220 | 0.002 |
| 221 | 0.039 |
| 222 | 0.002 |
| 223 | 0.022 |
| 224 | 0.009 |
| 225 | 0.001 |
| 226 | 0.028 |
| 227 | 0.008 |
| 228 | 0.020 |
| 229 | 0.003 |
| 230 | 0.025 |
| 231 | 0.092 |
| 232 | 0.012 |
| 233 | 0.038 |
| 234 | 0.017 |
| 235 | 0.030 |
| 236 | 0.044 |
| 237 | 0.019 |
| 238 | 0.018 |
| 239 | 0.024 |
| 240 | 0.026 |
| 241 | 0.010 |
| 242 | 0.052 |
| 243 | 0.076 |
| 244 | 0.014 |
| 245 | 0.017 |
| 246 | 0.008 |
| 247 | 0.046 |
| 248 | 0.010 |
| 249 | 0.002 |
| 250 | 0.012 |
| 251 | 0.083 |
| 252 | 0.010 |
| 253 | 0.015 |
| 254 | 0.083 |
| 255 | 0.023 |
| 256 | 0.019 |
| 257 | 0.035 |
| 258 | 0.034 |
| 259 | 0.016 |
| 260 | 0.009 |
| 261 | 0.041 |
| 262 | 0.098 |
| 263 | 0.030 |
| 264 | 0.008 |
| 265 | 0.070 |
| 266 | 0.072 |
| 267 | 0.092 |
| 268 | 0.072 |
| 269 | 0.028 |
| 270 | 0.054 |
| 271 | 0.020 |
| 272 | 0.058 |
| 273 | 0.004 |
| 274 | 0.044 |
| 275 | 0.002 |
| 276 | 0.002 |
| 277 | 0.012 |
| 278 | 0.005 |
| 279 | 0.055 |
| 280 | 0.018 |
| 281 | 0.010 |
| 282 | 0.011 |
| 283 | 0.005 |
| 284 | 0.009 |
| 285 | 0.012 |
| 286 | 0.008 |
| 287 | 0.008 |
| 288 | 0.058 |
| 289 | 0.084 |
| 290 | 0.006 |
| 291 | 0.056 |
| 292 | 0.016 |
| 293 | 0.096 |
| 294 | 0.011 |
| 295 | 0.026 |
| 296 | 0.030 |
| 297 | 0.036 |
| 298 | 0.003 |
| 299 | 0.002 |
| 300 | 0.057 |
| 301 | 0.028 |
| 302 | 0.018 |
| 303 | 0.027 |
| 304 | 0.033 |
| 305 | 0.013 |
| 306 | 0.045 |
| 307 | 0.065 |
| 308 | 0.093 |
| 309 | 0.043 |
| 310 | 0.091 |
| 311 | 0.054 |
| 312 | 0.071 |
| 313 | 0.074 |
| 314 | 0.074 |
| 315 | 0.060 |
| 316 | 0.010 |
| 317 | 0.030 |
| 318 | 0.043 |
| 319 | 0.021 |
| 320 | 0.034 |
| 321 | 0.031 |
| 322 | 0.059 |
| 323 | 0.005 |
| 324 | 0.040 |
| 325 | 0.042 |
| 326 | 0.023 |
| 327 | 0.050 |
| 328 | 0.020 |
| 329 | 0.048 |
| 330 | 0.046 |
| 331 | 0.081 |
| 332 | 0.044 |
| 333 | 0.035 |
| 334 | 0.014 |
| 335 | 0.070 |
| 336 | 0.012 |
| 337 | 0.069 |
| 338 | 0.041 |
| 339 | 0.020 |
| 340 | 0.063 |
| 341 | 0.001 |
| 342 | 0.034 |
| 343 | 0.017 |
| 344 | 0.010 |
| 345 | 0.017 |
| 346 | 0.045 |
| 347 | 0.002 |
| 348 | 0.013 |
| 349 | 0.006 |
| 350 | 0.077 |
| 351 | 0.029 |
| 352 | 0.005 |
| 353 | 0.004 |
| 354 | 0.034 |
| 355 | 0.025 |
| 356 | 0.024 |
| 357 | 0.019 |
| 358 | 0.035 |
| 359 | 0.083 |
| 360 | 0.021 |
| 361 | 0.020 |
| 362 | 0.018 |
| 363 | 0.019 |
| 364 | 0.025 |
| 365 | 0.056 |
| 366 | 0.026 |
| 367 | 0.002 |
| 368 | 0.082 |
| 369 | 0.072 |
| 370 | 0.010 |
| 371 | 0.030 |
| 372 | 0.015 |
| 373 | 0.007 |
| 374 | 0.100 |
| 375 | 0.053 |
| 376 | 0.066 |
| 377 | 0.011 |
| 378 | 0.004 |
| 379 | 0.003 |
| 380 | 0.019 |
| 381 | 0.003 |
| 382 | 0.008 |
| 383 | 0.006 |
| 384 | 0.000 |
| 385 | 0.045 |
| 386 | 0.017 |
| 387 | 0.025 |
| 388 | 0.004 |
| 389 | 0.023 |
| 390 | 0.020 |
| 391 | 0.005 |
| 392 | 0.008 |
| 393 | 0.021 |
| 394 | 0.004 |
| 395 | 0.011 |
| 396 | 0.040 |
| 397 | 0.014 |
| 398 | 0.027 |
| 399 | 0.020 |
| 400 | 0.071 |
| 401 | 0.076 |
| 402 | 0.013 |
| 403 | 0.020 |
| 404 | 0.028 |
| 405 | 0.068 |
| 406 | 0.028 |
| 407 | 0.013 |
| 408 | 0.010 |
| 409 | 0.033 |
| 410 | 0.001 |
| 411 | 0.009 |
| 412 | 0.002 |
| 413 | 0.005 |
| 414 | 0.024 |
| 415 | 0.021 |
| 416 | 0.011 |
| 417 | 0.002 |
| 418 | 0.002 |
| 419 | 0.001 |
| 420 | 0.017 |
| 421 | 0.003 |
| 422 | 0.002 |
| 423 | 0.013 |
| 424 | 0.002 |
| 425 | 0.003 |
| 426 | 0.004 |
| 427 | 0.006 |
| 428 | 0.002 |
| 429 | 0.006 |
| 430 | 0.012 |
| 431 | 0.002 |
| 432 | 0.011 |
| 433 | 0.003 |
| 434 | 0.008 |
| 435 | 0.002 |
| 436 | 0.014 |
| 437 | 0.003 |
| 438 | 0.011 |
| 439 | 0.003 |
| 440 | 0.029 |
| 441 | 0.012 |
| 442 | 0.013 |
| 443 | 0.042 |
| 444 | 0.056 |
| 445 | 0.012 |
| 446 | 0.003 |
| 447 | 0.011 |
| 448 | 0.004 |
| 449 | 0.005 |
| 450 | 0.004 |
| 451 | 0.007 |
| 452 | 0.004 |
| 453 | 0.010 |
| 454 | 0.008 |
| 455 | 0.033 |
| 456 | 0.006 |
| 457 | 0.005 |
| 458 | 0.040 |
| 459 | 0.043 |
| 460 | 0.031 |
| 461 | 0.023 |
| 462 | 0.006 |
| 463 | 0.070 |
| 464 | 0.071 |
| 465 | 0.033 |
| 466 | 0.024 |
| 467 | 0.022 |
| 468 | 0.030 |
| 469 | 0.032 |
| 470 | 0.003 |
| 471 | 0.007 |
| 472 | 0.016 |
| 473 | 0.003 |
| 474 | 0.026 |
| 475 | 0.015 |
| 476 | 0.007 |
| 477 | 0.005 |
| 478 | 0.003 |
| 479 | 0.012 |
| 480 | 0.010 |
| 481 | 0.007 |
| 482 | 0.022 |
| 483 | 0.003 |
| 484 | 0.025 |
| 485 | 0.015 |
| 486 | 0.016 |
| 487 | 0.004 |
| 488 | 0.029 |
| 489 | 0.051 |
| 490 | 0.007 |
| 491 | 0.043 |
| 492 | 0.048 |
| 493 | 0.035 |
| 494 | 0.034 |
| 494 | 0.011 |
| 496 | 0.015 |
| 497 | 0.035 |
| 498 | 0.049 |
| 499 | 0.013 |
| 500 | 0.020 |
| 501 | 0.064 |
| 502 | 0.059 |
| 503 | 0.011 |
| 504 | 0.035 |
| 505 | 0.033 |
| 506 | 0.044 |
| 507 | 0.046 |
| 508 | 0.063 |
| 509 | 0.031 |
| 510 | 0.043 |
| 511 | 0.023 |
| 512 | 0.039 |
| 513 | 0.045 |
| 514 | 0.040 |
| 515 | 0.060 |
| 516 | 0.075 |
| 517 | 0.022 |
| 518 | 0.014 |
| 519 | 0.031 |
| 520 | 0.027 |
| 521 | 0.062 |
| 522 | 0.019 |
| 523 | 0.016 |
| 524 | 0.099 |
| 525 | 0.014 |
| 526 | 0.053 |
| 527 | 0.034 |
| 528 | 0.016 |
| 529 | 0.032 |
| 530 | 0.100 |
| 531 | 0.021 |
| 532 | 0.033 |
| 533 | 0.006 |
| 534 | 0.027 |
| 535 | 0.021 |
| 536 | 0.068 |
| 537 | 0.097 |
| 538 | 0.076 |
| 539 | 0.006 |
| 540 | 0.002 |
| 541 | 0.001 |
| 542 | 0.001 |
| 543 | 0.002 |
| 544 | 0.003 |
| 545 | 0.001 |
| 546 | 0.002 |
| 547 | 0.004 |
| 548 | 0.003 |
| 549 | 0.002 |
| 550 | 0.002 |
| 551 | 0.002 |
| 552 | 0.001 |
| 553 | 0.002 |
| 554 | 0.001 |
| 555 | 0.002 |
| 556 | 0.003 |
| 557 | 0.002 |
| 558 | 0.002 |
| 559 | 0.003 |
| 560 | 0.003 |
| 561 | 0.002 |
| 562 | 0.003 |
| 563 | 0.016 |
| 564 | 0.002 |
| 565 | 0.010 |
| 566 | 0.002 |
| 567 | 0.004 |
| 568 | 0.002 |
| 569 | 0.002 |
| 570 | 0.003 |
| 571 | 0.003 |
| 572 | 0.002 |
| 573 | 0.002 |
| 574 | 0.006 |
| 575 | 0.011 |
| 576 | 0.001 |
| 577 | 0.002 |
| 578 | 0.002 |
| 579 | 0.003 |
| 580 | 0.001 |
| 581 | 0.001 |
| 582 | 0.001 |
| 583 | 0.001 |
| 584 | 0.004 |
| 585 | 0.002 |
| 586 | 0.009 |
| 587 | 0.006 |
| 588 | 0.004 |
| 589 | 0.001 |
| 590 | 0.002 |
| 591 | 0.001 |
| 592 | 0.001 |
| 593 | 0.004 |
| 594 | 0.003 |
| 595 | 0.002 |
| 596 | 0.012 |
| 597 | 0.003 |
| 598 | 0.001 |
| 599 | 0.001 |
| 600 | 0.001 |
| 601 | 0.001 |
| 602 | 0.001 |
| 603 | 0.001 |
| 604 | 0.003 |
| 605 | 0.005 |
| 606 | 0.007 |
| 607 | 0.008 |
| 608 | 0.007 |
| 609 | 0.005 |
| 610 | 0.008 |
| 611 | 0.001 |
| 612 | 0.001 |
| 613 | 0.004 |
| 614 | 0.001 |
| 615 | 0.002 |
| 616 | 0.007 |
| 617 | 0.002 |
| 618 | 0.003 |
| 619 | 0.010 |
| 620 | 0.005 |
| 621 | 0.004 |
| 622 | 0.009 |
| 623 | 0.001 |
| 624 | 0.007 |
| 625 | >10 |
| 626 | >10 |
| 627 | >10 |
| 628 | >10 |
| 629 | >10 |
| 630 | >10 |
| 631 | >10 |
| 632 | >10 |
| 633 | >10 |
| 634 | >10 |
| 635 | >10 |
| 636 | >10 |
| 637 | 0.008 |
| 638 | 0.007 |
| 639 | 0.040 |
| 640 | 0.029 |
| 641 | 0.040 |
| 642 | 0.016 |

### Example 3. CLK2 Kinase Activity Assay

Representative compounds were screened using the assay procedure for CLK2 kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, Calif.) into 1536-well black-walled round bottom plates (Corning).

The CLK2 kinase assay was run using the Ser/Thr 6 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant CLK2 kinase, ATP and Ser/Thr peptide 6 were prepared in 1× Kinase buffer to final concentrations of 0.43 μg/mL, 60 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 6 served as control reactions. Additionally, an 11-point dose-response curve of Staurosporine (1 μM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio×F100%)−C100%)/((C0%−C100%)+(Em ratio×(F100%−F0%)))]. Dose-response curves were generated and inhibitory concentration (IC₅₀) values were calculated using non-linear regression curve fit in the Dotmatics' Studies Software (Bishops Stortford, UK).

Table 6 shows the activity of representative compounds of Formulas (I)-(VIII) as provided herein.

TABLE 6

| Compound | EC₅₀ (μM) |
|---|---|
| 1 | 0.023 |
| 2 | 0.072 |
| 3 | 0.024 |
| 4 | 0.057 |
| 5 | 0.020 |
| 6 | 0.015 |
| 7 | 0.014 |
| 8 | 0.027 |
| 9 | 0.045 |
| 10 | 0.006 |
| 11 | 0.009 |
| 12 | 0.006 |
| 13 | 0.014 |
| 14 | 0.010 |
| 15 | 0.002 |
| 16 | 0.095 |
| 17 | 0.017 |
| 18 | 0.020 |
| 19 | 0.006 |
| 20 | 0.005 |
| 21 | 0.004 |
| 22 | 0.007 |
| 23 | 0.027 |
| 24 | 0.026 |
| 25 | 0.001 |
| 26 | 0.001 |
| 27 | 0.032 |
| 28 | 0.046 |
| 29 | 0.025 |
| 30 | 0.018 |
| 31 | 0.028 |
| 32 | 0.038 |
| 33 | 0.003 |
| 34 | 0.014 |
| 35 | 0.002 |
| 36 | 0.070 |
| 37 | 0.043 |
| 38 | 0.035 |
| 39 | 0.025 |
| 40 | 0.045 |
| 41 | 0.001 |
| 42 | 0.001 |
| 43 | 0.001 |
| 44 | 0.001 |
| 45 | 0.005 |
| 46 | 0.001 |
| 47 | 0.003 |
| 48 | 0.003 |
| 49 | 0.006 |
| 50 | 0.027 |
| 51 | 0.037 |
| 52 | 0.002 |
| 53 | 0.019 |
| 54 | 0.001 |
| 55 | 0.001 |
| 56 | 0.004 |
| 57 | 0.001 |
| 58 | 0.010 |
| 59 | 0.010 |
| 60 | 0.002 |
| 61 | 0.008 |
| 62 | 0.001 |
| 63 | 0.001 |
| 64 | 0.001 |
| 65 | 0.001 |
| 66 | 0.001 |
| 67 | 0.001 |
| 68 | 0.001 |
| 69 | 0.002 |
| 70 | 0.001 |
| 71 | 0.002 |
| 72 | 0.002 |
| 73 | 0.001 |
| 74 | 0.012 |
| 75 | 0.013 |
| 76 | 0.008 |
| 77 | 0.006 |
| 78 | 0.001 |
| 79 | 0.001 |
| 80 | 0.003 |
| 81 | 0.001 |
| 82 | 0.013 |
| 83 | 0.005 |
| 84 | 0.001 |
| 85 | 0.007 |
| 86 | 0.003 |
| 87 | 0.001 |
| 88 | 0.001 |
| 89 | 0.001 |
| 90 | 0.001 |
| 91 | 0.001 |
| 92 | 0.001 |
| 93 | 0.005 |
| 94 | 0.001 |
| 95 | 0.001 |
| 96 | 0.001 |
| 97 | 0.001 |
| 98 | 0.001 |
| 99 | 0.002 |
| 100 | 0.001 |
| 101 | 0.001 |
| 102 | 0.002 |
| 103 | 0.002 |
| 104 | 0.002 |
| 105 | 0.001 |
| 106 | 0.001 |
| 107 | 0.001 |
| 108 | 0.001 |
| 109 | 0.001 |
| 110 | 0.001 |
| 111 | 0.001 |
| 112 | 0.001 |
| 113 | 0.001 |
| 114 | 0.001 |
| 115 | 0.001 |
| 116 | 0.001 |
| 117 | 0.002 |
| 118 | 0.008 |
| 119 | 0.005 |
| 120 | 0.023 |
| 121 | 0.003 |
| 122 | 0.011 |
| 123 | 0.006 |
| 124 | 0.037 |
| 125 | 0.001 |
| 126 | 0.012 |
| 127 | 0.006 |
| 128 | 0.002 |
| 129 | 0.053 |
| 130 | 0.003 |
| 131 | 0.013 |
| 132 | 0.001 |
| 133 | 0.002 |
| 134 | 0.006 |
| 135 | 0.004 |
| 136 | 0.002 |
| 137 | 0.001 |
| 138 | 0.001 |
| 139 | 0.003 |
| 140 | 0.002 |
| 141 | 0.009 |
| 142 | 0.021 |
| 143 | 0.017 |
| 144 | 0.005 |
| 145 | 0.005 |
| 146 | 0.005 |
| 147 | 0.009 |
| 148 | 0.004 |
| 149 | 0.002 |
| 150 | 0.003 |
| 151 | 0.001 |
| 152 | 0.001 |
| 153 | 0.011 |
| 154 | 0.003 |
| 155 | 0.014 |
| 156 | 0.010 |
| 157 | 0.002 |
| 158 | 0.003 |
| 159 | 0.001 |
| 160 | 0.003 |
| 161 | 0.017 |
| 162 | 0.008 |
| 163 | 0.032 |
| 164 | 0.028 |
| 165 | 0.002 |
| 166 | 0.003 |
| 167 | 0.004 |
| 168 | 0.033 |
| 169 | 0.001 |
| 170 | 0.001 |
| 171 | 0.001 |
| 172 | 0.007 |
| 173 | 0.004 |
| 174 | 0.004 |
| 175 | 0.005 |
| 176 | 0.006 |
| 177 | 0.006 |
| 178 | 0.005 |
| 179 | 0.011 |
| 180 | 0.002 |
| 181 | 0.015 |
| 182 | 0.011 |
| 183 | 0.001 |
| 184 | 0.001 |
| 185 | 0.001 |
| 186 | 0.004 |
| 187 | 0.002 |
| 188 | 0.003 |
| 189 | 0.008 |
| 190 | 0.005 |
| 191 | 0.003 |
| 192 | 0.005 |
| 193 | 0.002 |
| 194 | 0.002 |
| 195 | 0.001 |
| 196 | 0.005 |
| 198 | 0.011 |
| 199 | 0.002 |
| 200 | 0.022 |
| 201 | 0.015 |
| 202 | 0.002 |
| 203 | 0.011 |
| 204 | 0.027 |
| 205 | 0.001 |
| 206 | 0.012 |
| 207 | 0.003 |
| 208 | 0.004 |
| 209 | 0.011 |
| 210 | 0.007 |
| 211 | 0.008 |
| 212 | 0.005 |
| 213 | 0.001 |
| 214 | 0.002 |
| 215 | 0.005 |
| 216 | 0.002 |
| 217 | 0.003 |
| 218 | 0.001 |
| 219 | 0.001 |
| 220 | 0.001 |
| 221 | 0.003 |
| 222 | 0.003 |
| 223 | 0.003 |
| 224 | 0.012 |
| 225 | 0.002 |
| 226 | 0.004 |
| 227 | 0.010 |
| 228 | 0.004 |
| 229 | 0.002 |
| 230 | 0.004 |
| 231 | 0.003 |
| 232 | 0.005 |
| 233 | 0.008 |
| 234 | 0.006 |
| 235 | 0.008 |
| 236 | 0.019 |
| 237 | 0.007 |
| 238 | 0.002 |
| 239 | 0.002 |
| 240 | 0.002 |
| 241 | 0.004 |
| 242 | 0.015 |
| 243 | 0.005 |
| 244 | 0.016 |
| 245 | 0.034 |
| 246 | 0.003 |
| 247 | 0.002 |
| 248 | 0.001 |
| 249 | 0.001 |
| 250 | 0.004 |
| 251 | 0.000 |
| 252 | 0.002 |
| 253 | 0.007 |
| 254 | 0.003 |
| 255 | 0.003 |
| 256 | 0.009 |
| 257 | 0.007 |
| 258 | 0.002 |
| 259 | 0.004 |
| 260 | 0.001 |
| 261 | 0.002 |
| 262 | 0.085 |
| 263 | 0.018 |
| 264 | 0.002 |
| 265 | 0.008 |
| 266 | 0.015 |
| 267 | 0.008 |
| 268 | 0.012 |
| 269 | 0.012 |
| 270 | 0.004 |
| 271 | 0.002 |
| 272 | 0.005 |
| 273 | 0.004 |
| 274 | 0.011 |
| 275 | 0.001 |
| 276 | 0.002 |
| 277 | 0.006 |
| 278 | 0.005 |
| 279 | 0.002 |
| 280 | 0.002 |
| 281 | 0.006 |
| 282 | 0.004 |
| 283 | 0.004 |
| 284 | 0.001 |
| 285 | 0.001 |
| 286 | 0.003 |
| 287 | 0.004 |
| 288 | 0.011 |
| 289 | 0.004 |
| 290 | 0.002 |
| 291 | 0.032 |
| 292 | 0.009 |
| 293 | 0.016 |
| 294 | 0.001 |
| 295 | 0.002 |
| 296 | 0.001 |
| 297 | 0.001 |
| 298 | 0.001 |
| 299 | 0.001 |
| 300 | 0.008 |
| 301 | 0.008 |
| 302 | 0.006 |
| 303 | 0.015 |
| 304 | 0.016 |
| 305 | 0.003 |
| 306 | 0.008 |
| 307 | 0.013 |
| 308 | 0.007 |
| 309 | 0.003 |
| 310 | 0.004 |
| 311 | 0.003 |
| 312 | 0.004 |
| 313 | 0.018 |
| 314 | 0.010 |
| 315 | 0.003 |
| 316 | 0.007 |
| 317 | 0.016 |
| 318 | 0.019 |
| 319 | 0.006 |
| 320 | 0.009 |
| 321 | 0.007 |
| 322 | 0.006 |
| 323 | 0.002 |
| 324 | 0.006 |
| 325 | 0.004 |
| 326 | 0.010 |
| 327 | 0.010 |
| 328 | 0.006 |
| 329 | 0.008 |
| 330 | 0.012 |
| 331 | 0.015 |
| 332 | 0.006 |
| 333 | 0.020 |
| 334 | 0.002 |
| 335 | 0.019 |
| 336 | 0.003 |
| 337 | 0.002 |
| 338 | 0.005 |
| 339 | 0.007 |
| 340 | 0.010 |
| 341 | 0.000 |
| 342 | 0.004 |
| 343 | 0.003 |
| 344 | 0.003 |
| 345 | 0.003 |
| 346 | 0.004 |
| 347 | 0.002 |
| 348 | 0.003 |
| 349 | 0.002 |
| 350 | 0.011 |
| 351 | 0.004 |
| 352 | 0.003 |
| 353 | 0.003 |
| 354 | 0.012 |
| 355 | 0.008 |
| 356 | 0.006 |
| 357 | 0.003 |
| 358 | 0.007 |
| 359 | 0.006 |
| 360 | 0.004 |
| 361 | 0.005 |
| 362 | 0.004 |
| 363 | 0.008 |
| 364 | 0.010 |
| 365 | 0.013 |
| 366 | 0.004 |
| 367 | 0.002 |
| 368 | 0.004 |
| 369 | 0.009 |
| 370 | 0.006 |
| 371 | 0.011 |
| 372 | 0.002 |
| 373 | 0.004 |
| 374 | 0.019 |
| 375 | 0.006 |
| 376 | 0.011 |
| 377 | 0.007 |
| 378 | 0.002 |
| 379 | 0.004 |
| 380 | 0.005 |
| 381 | 0.003 |
| 382 | 0.004 |
| 383 | 0.003 |
| 384 | 0.000 |
| 385 | 0.012 |
| 386 | 0.008 |
| 387 | 0.005 |
| 388 | 0.004 |
| 389 | 0.009 |
| 390 | 0.011 |
| 391 | 0.004 |
| 392 | 0.009 |
| 393 | 0.006 |
| 394 | 0.003 |
| 395 | 0.005 |
| 396 | 0.012 |
| 397 | 0.003 |
| 398 | 0.003 |
| 399 | 0.004 |
| 400 | 0.012 |
| 401 | 0.015 |
| 402 | 0.005 |
| 403 | 0.006 |
| 404 | 0.005 |
| 405 | 0.003 |
| 406 | 0.003 |
| 407 | 0.003 |
| 408 | 0.002 |
| 409 | 0.005 |
| 410 | 0.001 |
| 411 | 0.005 |
| 412 | 0.002 |
| 413 | 0.004 |
| 414 | 0.003 |
| 415 | 0.001 |
| 416 | 0.003 |
| 417 | 0.003 |
| 418 | 0.002 |
| 419 | 0.001 |
| 420 | 0.011 |
| 421 | 0.003 |
| 422 | 0.003 |
| 423 | 0.021 |
| 424 | 0.002 |
| 425 | 0.003 |
| 426 | 0.005 |
| 427 | 0.009 |
| 428 | 0.002 |
| 429 | 0.009 |
| 430 | 0.007 |
| 431 | 0.001 |
| 432 | 0.017 |
| 433 | 0.003 |
| 434 | 0.005 |
| 435 | 0.002 |
| 436 | 0.008 |
| 437 | 0.002 |
| 438 | 0.004 |
| 439 | 0.002 |
| 440 | 0.003 |
| 441 | 0.005 |
| 442 | 0.003 |
| 443 | 0.007 |
| 444 | 0.009 |
| 445 | 0.005 |
| 446 | 0.001 |
| 447 | 0.002 |
| 448 | 0.003 |
| 449 | 0.002 |
| 450 | 0.003 |
| 451 | 0.005 |
| 452 | 0.002 |
| 453 | 0.002 |
| 454 | 0.003 |
| 455 | 0.005 |
| 456 | 0.002 |
| 457 | 0.001 |
| 458 | 0.020 |
| 459 | 0.013 |
| 460 | 0.008 |
| 461 | 0.019 |
| 462 | 0.002 |
| 463 | 0.013 |
| 464 | 0.013 |
| 465 | 0.002 |
| 466 | 0.003 |
| 467 | 0.012 |
| 468 | 0.005 |
| 469 | 0.006 |
| 470 | 0.002 |
| 471 | 0.003 |
| 472 | 0.010 |
| 473 | 0.003 |
| 474 | 0.017 |
| 475 | 0.011 |
| 476 | 0.007 |
| 477 | 0.003 |
| 478 | 0.009 |
| 479 | 0.004 |
| 480 | 0.007 |
| 481 | 0.009 |
| 482 | 0.014 |
| 483 | 0.008 |
| 484 | 0.006 |
| 485 | 0.004 |
| 486 | 0.006 |
| 487 | 0.005 |
| 488 | 0.003 |
| 489 | 0.019 |
| 490 | 0.003 |
| 491 | 0.020 |
| 492 | 0.012 |
| 493 | 0.006 |
| 494 | 0.012 |
| 494 | 0.009 |
| 496 | 0.006 |
| 497 | 0.005 |
| 498 | 0.003 |
| 499 | 0.003 |
| 500 | 0.012 |
| 501 | 0.011 |
| 502 | 0.014 |
| 503 | 0.010 |
| 504 | 0.006 |
| 505 | 0.004 |
| 506 | 0.008 |
| 507 | 0.010 |
| 508 | 0.007 |
| 509 | 0.006 |
| 510 | 0.016 |
| 511 | 0.006 |
| 512 | 0.014 |
| 513 | 0.006 |
| 514 | 0.004 |
| 515 | 0.005 |
| 516 | 0.005 |
| 517 | 0.008 |
| 518 | 0.007 |
| 519 | 0.018 |
| 520 | 0.003 |
| 521 | 0.009 |
| 522 | 0.008 |
| 523 | 0.009 |
| 524 | 0.015 |
| 525 | 0.008 |
| 526 | 0.004 |
| 527 | 0.011 |
| 528 | 0.005 |
| 529 | 0.008 |
| 530 | 0.017 |
| 531 | 0.005 |
| 532 | 0.003 |
| 533 | 0.002 |
| 534 | 0.004 |
| 535 | 0.006 |
| 536 | 0.005 |
| 537 | 0.013 |
| 538 | 0.002 |
| 539 | 0.007 |
| 540 | 0.003 |
| 541 | 0.001 |
| 542 | 0.001 |
| 543 | 0.003 |
| 544 | 0.002 |
| 545 | 0.001 |
| 546 | 0.002 |
| 547 | 0.003 |
| 548 | 0.002 |
| 549 | 0.003 |
| 550 | 0.002 |
| 551 | 0.010 |
| 552 | 0.001 |
| 553 | 0.002 |
| 554 | 0.001 |
| 555 | 0.001 |
| 556 | 0.002 |
| 557 | 0.002 |
| 558 | 0.003 |
| 559 | 0.004 |
| 560 | 0.004 |
| 561 | 0.001 |
| 562 | 0.001 |
| 563 | 0.005 |
| 564 | 0.002 |
| 565 | 0.006 |
| 566 | 0.002 |
| 567 | 0.008 |
| 568 | 0.003 |
| 569 | 0.008 |
| 570 | 0.009 |
| 571 | 0.007 |
| 572 | 0.002 |
| 573 | 0.002 |
| 574 | 0.002 |
| 575 | 0.002 |
| 576 | 0.002 |
| 577 | 0.003 |
| 578 | 0.002 |
| 579 | 0.002 |
| 580 | 0.001 |
| 581 | 0.002 |
| 582 | 0.002 |
| 583 | 0.002 |
| 584 | 0.006 |
| 585 | 0.001 |
| 586 | 0.007 |
| 587 | 0.008 |
| 588 | 0.002 |
| 589 | 0.002 |
| 590 | 0.004 |
| 591 | 0.001 |
| 592 | 0.002 |
| 593 | 0.003 |
| 594 | 0.002 |
| 595 | 0.002 |
| 596 | 0.001 |
| 597 | 0.009 |
| 598 | 0.008 |
| 599 | 0.002 |
| 600 | 0.003 |
| 601 | 0.002 |
| 602 | 0.002 |
| 603 | 0.001 |
| 604 | 0.002 |
| 605 | 0.002 |
| 606 | 0.004 |
| 607 | 0.006 |
| 608 | 0.008 |
| 609 | 0.006 |
| 610 | 0.009 |
| 611 | 0.003 |
| 612 | 0.005 |
| 613 | 0.007 |
| 614 | 0.007 |
| 615 | 0.007 |
| 616 | 0.006 |
| 617 | 0.002 |
| 618 | 0.002 |
| 619 | 0.002 |
| 620 | 0.003 |
| 621 | 0.003 |
| 622 | 0.005 |
| 623 | 0.002 |
| 624 | 0.010 |
| 625 | 0.005 |
| 626 | 0.007 |
| 627 | 0.007 |
| 628 | 0.005 |
| 629 | 0.009 |
| 630 | 0.008 |
| 631 | 0.010 |
| 632 | 0.003 |
| 633 | 0.003 |
| 634 | 0.008 |
| 635 | 0.010 |
| 636 | 0.009 |
| 637 | 1.902 |
| 638 | 1.133 |
| 639 | >10 |
| 640 | 7.429 |
| 641 | >10 |
| 642 | 9.777 |

### Example 4. CLK3 Kinase Activity Assay

Representative compounds were screened using the assay procedure for CLK3 kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, Calif.) into 1536-well black-walled round bottom plates (Corning).

The CLK3 kinase assay was run using the Ser/Thr 18 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant CLK3 kinase, ATP and Ser/Thr peptide 18 were prepared in 1× Kinase buffer to final concentrations of 1.5 μg/mL, 156 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (1 μM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio×F100%)−C100%)/((C0%−C100%)+(Em ratio×(F100%−F0%)))]. Dose-response curves were generated and inhibitory concentration (IC₅₀) values were calculated using non-linear regression curve fit in the Dotmatics' Studies Software (Bishops Stortford, UK).

Table 7 shows the activity of representative compounds of Formulas (I)-(VIII) as provided herein.

TABLE 7

| Compound | EC₅₀ (μM) |
|---|---|
| 1 | 5.302 |
| 2 | >10 |
| 3 | 3.700 |
| 4 | 6.607 |
| 5 | 4.927 |
| 6 | >10 |
| 7 | 2.355 |
| 8 | 6.159 |
| 9 | 6.088 |
| 10 | 1.779 |
| 11 | 5.154 |
| 12 | 6.020 |
| 13 | 3.503 |
| 14 | 4.305 |
| 15 | 0.485 |
| 16 | >10 |
| 17 | 2.671 |
| 18 | 3.989 |
| 19 | 0.967 |
| 20 | 0.563 |
| 21 | 0.733 |
| 22 | 2.328 |
| 23 | 0.627 |
| 24 | 1.252 |
| 25 | 0.326 |
| 26 | 0.492 |
| 27 | >10 |
| 28 | >10 |
| 29 | >10 |
| 30 | >10 |
| 31 | >10 |
| 32 | >10 |
| 33 | 0.148 |
| 34 | 5.248 |
| 35 | 0.084 |
| 36 | 3.400 |
| 37 | 5.500 |
| 38 | 8.008 |
| 39 | 4.261 |
| 40 | 2.380 |
| 41 | 1.420 |
| 42 | 0.424 |
| 43 | 0.309 |
| 44 | 1.720 |
| 45 | 1.760 |
| 46 | 1.300 |
| 47 | 0.313 |
| 48 | 1.100 |
| 49 | 2.204 |
| 50 | 5.731 |
| 51 | 3.950 |
| 52 | 0.361 |
| 53 | 3.493 |
| 54 | 0.020 |
| 55 | 0.013 |
| 56 | 0.047 |
| 57 | 0.081 |
| 58 | 9.838 |
| 59 | 6.145 |
| 60 | 0.581 |
| 61 | 0.916 |
| 62 | 0.034 |
| 63 | 0.343 |
| 64 | 0.021 |
| 65 | 0.047 |
| 66 | 0.049 |
| 67 | 0.041 |
| 68 | 0.139 |
| 69 | 0.062 |
| 70 | 0.036 |
| 71 | 0.109 |
| 72 | 0.022 |
| 73 | 0.011 |
| 74 | 0.498 |
| 75 | 4.024 |
| 76 | 0.717 |
| 77 | 1.674 |
| 78 | 0.044 |
| 79 | 0.352 |
| 80 | 0.093 |
| 81 | 0.032 |
| 82 | 0.616 |
| 83 | 0.073 |
| 84 | 0.014 |
| 85 | 1.805 |
| 86 | 1.273 |
| 87 | 0.011 |
| 88 | 0.012 |
| 89 | 0.014 |
| 90 | 0.039 |
| 91 | 0.022 |
| 92 | 0.089 |
| 93 | 0.071 |
| 94 | 0.011 |
| 95 | 0.046 |
| 96 | 0.017 |
| 97 | 0.060 |
| 98 | 0.086 |
| 99 | 0.135 |
| 100 | 0.037 |
| 101 | 0.044 |
| 102 | 0.088 |
| 103 | 1.434 |
| 104 | 0.378 |
| 105 | 2.440 |
| 106 | 0.055 |
| 107 | 0.090 |
| 108 | 0.101 |
| 109 | 0.079 |
| 110 | 0.048 |
| 111 | 0.089 |
| 112 | 0.116 |
| 113 | 0.133 |
| 114 | 0.048 |
| 115 | 0.099 |
| 116 | 0.391 |
| 117 | 0.147 |
| 118 | 0.601 |
| 119 | 0.048 |
| 120 | 0.061 |
| 121 | 0.030 |
| 122 | 6.956 |
| 123 | 0.056 |
| 124 | 0.218 |
| 125 | 0.057 |
| 126 | 7.330 |
| 127 | 0.042 |
| 128 | 0.028 |
| 129 | 0.164 |
| 130 | 0.018 |
| 131 | 0.160 |
| 132 | 0.012 |
| 133 | 0.037 |
| 134 | 0.076 |
| 135 | 0.041 |
| 136 | 0.109 |
| 137 | 0.007 |
| 138 | 0.197 |
| 139 | 0.079 |
| 140 | 0.022 |
| 141 | 0.033 |
| 142 | 0.090 |
| 143 | 0.045 |
| 144 | 0.093 |
| 145 | 0.074 |
| 146 | 0.113 |
| 147 | 0.493 |
| 148 | 0.124 |
| 149 | 0.065 |
| 150 | 0.089 |
| 151 | 0.055 |
| 152 | 0.033 |
| 153 | 0.093 |
| 154 | >10 |
| 155 | 0.138 |
| 156 | 1.800 |
| 157 | 0.416 |
| 158 | 0.083 |
| 159 | 0.034 |
| 160 | 0.037 |
| 161 | 0.105 |
| 162 | 9.838 |
| 163 | 0.075 |
| 164 | 0.087 |
| 165 | 0.020 |
| 166 | 0.209 |
| 167 | 0.019 |
| 168 | 0.076 |
| 169 | 0.050 |
| 170 | 0.012 |
| 171 | 0.034 |
| 172 | 0.117 |
| 173 | 0.024 |
| 174 | 9.838 |
| 175 | 0.180 |
| 176 | 0.149 |
| 177 | 0.209 |
| 178 | 0.207 |
| 179 | 0.969 |
| 180 | 0.296 |
| 181 | 0.017 |
| 182 | 9.838 |
| 183 | 0.105 |
| 184 | 0.177 |
| 185 | 0.308 |
| 186 | 0.037 |
| 187 | 0.132 |
| 188 | 0.023 |
| 189 | 0.026 |
| 190 | 0.039 |
| 191 | 0.049 |
| 192 | 0.017 |
| 193 | 0.322 |
| 194 | 0.213 |
| 195 | 0.280 |
| 196 | 0.995 |
| 198 | 0.984 |
| 199 | 0.136 |
| 200 | 0.209 |
| 201 | 2.269 |
| 202 | 0.065 |
| 203 | 0.558 |
| 204 | 0.206 |
| 205 | 0.182 |
| 206 | 0.213 |
| 207 | 1.292 |
| 208 | 0.058 |
| 209 | 0.094 |
| 210 | 0.054 |
| 211 | 0.107 |
| 212 | 0.089 |
| 213 | 0.051 |
| 214 | 0.344 |
| 215 | 0.500 |
| 216 | 0.843 |
| 217 | 0.040 |
| 218 | 0.032 |
| 219 | 0.026 |
| 220 | 0.061 |
| 221 | 0.188 |
| 222 | 0.263 |
| 223 | 0.123 |
| 224 | 0.056 |
| 225 | 0.016 |
| 226 | 0.116 |
| 227 | 1.305 |
| 228 | 0.106 |
| 229 | 0.044 |
| 230 | 0.087 |
| 231 | 0.361 |
| 232 | 0.143 |
| 233 | 0.521 |
| 234 | 0.120 |
| 235 | 2.629 |
| 236 | 0.205 |
| 237 | 0.134 |
| 238 | 0.230 |
| 239 | 0.087 |
| 240 | 0.113 |
| 241 | 0.034 |
| 242 | 0.076 |
| 243 | 0.062 |
| 244 | 0.064 |
| 245 | 9.838 |
| 246 | 0.021 |
| 247 | 0.048 |
| 248 | 0.066 |
| 249 | 0.030 |
| 250 | >10 |
| 251 | 0.067 |
| 252 | 0.118 |
| 253 | 0.026 |
| 254 | 0.043 |
| 255 | 0.062 |
| 256 | 0.062 |
| 257 | 0.115 |
| 258 | 0.083 |
| 259 | 0.167 |
| 260 | 0.128 |
| 261 | 0.150 |
| 262 | 0.754 |
| 263 | 0.358 |
| 264 | 0.035 |
| 265 | 0.091 |
| 266 | 0.067 |
| 267 | 9.838 |
| 268 | 0.145 |
| 269 | 0.183 |
| 270 | 0.088 |
| 271 | 0.034 |
| 272 | 0.052 |
| 273 | 0.029 |
| 274 | 0.203 |
| 275 | 0.020 |
| 276 | 0.009 |
| 277 | 0.029 |
| 278 | 0.036 |
| 279 | 0.093 |
| 280 | 0.079 |
| 281 | 0.025 |
| 282 | 0.050 |
| 283 | 0.033 |
| 284 | 0.025 |
| 285 | 0.090 |
| 286 | 0.082 |
| 287 | 0.044 |
| 288 | 0.037 |
| 289 | 0.039 |
| 290 | 0.017 |
| 291 | 0.054 |
| 292 | 0.060 |
| 293 | 0.186 |
| 294 | 0.025 |
| 295 | 0.096 |
| 296 | 0.055 |
| 297 | 0.084 |
| 298 | 0.013 |
| 299 | 0.029 |
| 300 | 0.122 |
| 301 | 0.105 |
| 302 | 0.046 |
| 303 | 0.085 |
| 304 | 0.021 |
| 305 | 0.033 |
| 306 | 0.138 |
| 307 | 0.307 |
| 308 | 0.083 |
| 309 | 0.037 |
| 310 | 0.074 |
| 311 | 0.218 |
| 312 | 0.252 |
| 313 | 0.295 |
| 314 | 0.202 |
| 315 | 0.139 |
| 316 | 0.417 |
| 317 | 9.838 |
| 318 | >10 |
| 319 | 0.817 |
| 320 | 0.033 |
| 321 | 0.067 |
| 322 | 0.066 |
| 323 | 0.059 |
| 324 | 0.205 |
| 325 | >10 |
| 326 | 0.144 |
| 327 | 0.414 |
| 328 | 0.047 |
| 329 | 0.042 |
| 330 | 0.109 |
| 331 | 0.158 |
| 332 | 0.196 |
| 333 | >10 |
| 334 | 0.089 |
| 335 | 2.313 |
| 336 | 0.107 |
| 337 | 0.109 |
| 338 | 0.222 |
| 339 | 0.099 |
| 340 | 0.785 |
| 341 | 0.003 |
| 342 | 0.090 |
| 343 | 0.468 |
| 344 | 9.838 |
| 345 | 0.107 |
| 346 | 0.133 |
| 347 | 0.018 |
| 348 | 0.538 |
| 349 | 0.037 |
| 350 | 1.905 |
| 351 | 9.838 |
| 352 | 9.838 |
| 353 | 0.085 |
| 354 | 0.537 |
| 355 | 0.151 |
| 356 | 0.078 |
| 357 | 0.043 |
| 358 | 0.071 |
| 359 | 0.962 |
| 360 | 0.330 |
| 361 | 0.164 |
| 362 | 0.292 |
| 363 | 9.838 |
| 364 | 0.139 |
| 365 | 0.443 |
| 366 | 0.074 |
| 367 | 0.214 |
| 368 | 0.102 |
| 369 | 0.102 |
| 370 | 0.101 |
| 371 | 0.408 |
| 372 | 0.069 |
| 373 | 0.156 |
| 374 | 9.838 |
| 375 | 0.100 |
| 376 | >10 |
| 377 | 0.113 |
| 378 | 0.050 |
| 379 | 0.069 |
| 380 | >10 |
| 381 | >10 |
| 382 | 0.063 |
| 383 | 0.046 |
| 384 | 0.100 |
| 385 | 0.389 |
| 386 | 0.201 |
| 387 | 0.132 |
| 388 | 0.029 |
| 389 | 9.914 |
| 390 | 0.137 |
| 391 | 0.062 |
| 392 | 0.092 |
| 393 | >10 |
| 394 | 0.069 |
| 395 | 0.030 |
| 396 | >10 |
| 397 | >10 |
| 398 | 0.056 |
| 399 | 0.188 |
| 400 | 0.180 |
| 401 | 0.895 |
| 402 | 0.110 |
| 403 | 0.306 |
| 404 | >10 |
| 405 | >10 |
| 406 | 0.227 |
| 407 | 0.019 |
| 408 | 0.032 |
| 409 | 0.142 |
| 410 | 0.015 |
| 411 | 0.351 |
| 412 | 0.088 |
| 413 | 5.488 |
| 414 | 1.360 |
| 415 | 0.031 |
| 416 | 0.061 |
| 417 | 0.073 |
| 418 | 0.041 |
| 419 | 0.016 |
| 420 | 0.344 |
| 421 | 0.083 |
| 422 | 0.054 |
| 423 | 6.212 |
| 424 | 0.047 |
| 425 | 0.056 |
| 426 | 0.088 |
| 427 | 9.838 |
| 428 | 0.019 |
| 429 | 0.365 |
| 430 | 0.147 |
| 431 | 0.018 |
| 432 | 2.971 |
| 433 | 0.062 |
| 434 | 0.189 |
| 435 | 0.046 |
| 436 | >10 |
| 437 | 0.062 |
| 438 | 0.269 |
| 439 | 0.049 |
| 440 | 0.562 |
| 441 | 0.538 |
| 442 | 0.101 |
| 443 | 0.566 |
| 444 | 0.362 |
| 445 | 0.203 |
| 446 | 0.023 |
| 447 | 0.037 |
| 448 | 0.028 |
| 449 | 0.019 |
| 450 | 0.085 |
| 451 | 0.053 |
| 452 | 0.068 |
| 453 | 0.246 |
| 454 | >10 |
| 455 | 0.110 |
| 456 | 0.094 |
| 457 | 0.052 |
| 458 | 2.192 |
| 459 | 0.269 |
| 460 | 0.231 |
| 461 | 0.342 |
| 462 | 0.048 |
| 463 | 1.277 |
| 464 | 3.662 |
| 465 | 0.293 |
| 466 | 0.064 |
| 467 | 0.279 |
| 468 | 0.166 |
| 469 | 0.470 |
| 470 | 0.040 |
| 471 | 0.089 |
| 472 | 1.082 |
| 473 | 0.285 |
| 474 | 2.039 |
| 475 | 0.569 |
| 476 | 0.800 |
| 477 | 0.204 |
| 478 | 0.137 |
| 479 | 0.145 |
| 480 | 0.186 |
| 481 | 0.741 |
| 482 | 2.378 |
| 483 | 0.303 |
| 484 | 0.428 |
| 485 | 0.303 |
| 486 | 0.291 |
| 487 | 0.302 |
| 488 | 2.528 |
| 489 | 0.340 |
| 490 | 0.142 |
| 491 | 1.938 |
| 492 | 0.885 |
| 493 | 0.912 |
| 494 | 0.973 |
| 494 | 1.901 |
| 496 | 0.524 |
| 497 | 0.678 |
| 498 | >10 |
| 499 | 0.042 |
| 500 | 0.117 |
| 501 | 0.418 |
| 502 | 0.336 |
| 503 | 0.104 |
| 504 | 0.117 |
| 505 | 0.217 |
| 506 | 0.392 |
| 507 | 0.060 |
| 508 | 0.068 |
| 509 | 0.140 |
| 510 | 1.867 |
| 511 | 0.050 |
| 512 | 0.094 |
| 513 | 0.049 |
| 514 | 0.049 |
| 515 | 0.109 |
| 516 | 0.063 |
| 517 | 0.216 |
| 518 | 0.039 |
| 519 | 0.236 |
| 520 | 0.075 |
| 521 | 0.776 |
| 522 | 0.048 |
| 523 | 0.081 |
| 524 | 0.198 |
| 525 | 0.038 |
| 526 | 0.140 |
| 527 | 0.066 |
| 528 | 0.059 |
| 529 | 0.188 |
| 530 | 0.151 |
| 531 | 0.052 |
| 532 | 0.305 |
| 533 | 0.051 |
| 534 | 0.471 |
| 535 | 0.053 |
| 536 | 0.079 |
| 537 | 0.329 |
| 538 | 0.058 |
| 539 | 0.123 |
| 540 | 0.041 |
| 541 | 0.014 |
| 542 | 0.036 |
| 543 | 0.060 |
| 544 | 0.031 |
| 545 | 0.012 |
| 546 | 0.021 |
| 547 | 0.043 |
| 548 | 0.026 |
| 549 | 0.026 |
| 550 | 0.028 |
| 551 | 0.078 |
| 552 | 0.009 |
| 553 | 0.280 |
| 554 | 0.016 |
| 555 | 0.021 |
| 556 | 0.038 |
| 557 | 0.039 |
| 558 | 0.049 |
| 559 | 0.066 |
| 560 | 0.085 |
| 561 | 0.006 |
| 562 | 0.020 |
| 563 | 0.026 |
| 564 | 0.037 |
| 565 | 0.071 |
| 566 | 0.034 |
| 567 | 0.261 |
| 568 | 0.044 |
| 569 | 0.164 |
| 570 | 0.143 |
| 571 | 0.318 |
| 572 | 0.015 |
| 573 | 0.008 |
| 574 | 0.200 |
| 575 | 0.026 |
| 576 | 0.081 |
| 577 | 0.056 |
| 578 | 0.048 |
| 579 | 0.019 |
| 580 | 0.033 |
| 581 | 0.011 |
| 582 | 0.018 |
| 583 | 0.027 |
| 584 | 0.177 |
| 585 | 0.013 |
| 586 | 0.023 |
| 587 | 0.678 |
| 588 | 0.070 |
| 589 | 0.031 |
| 590 | 0.040 |
| 591 | 0.020 |
| 592 | 0.043 |
| 593 | 0.022 |
| 594 | 0.014 |
| 595 | 0.019 |
| 596 | 0.042 |
| 597 | 0.094 |
| 598 | 0.052 |
| 599 | 0.011 |
| 600 | 0.078 |
| 601 | 0.059 |
| 602 | 0.018 |
| 603 | 0.011 |
| 604 | 0.007 |
| 605 | 0.009 |
| 606 | 0.060 |
| 607 | 0.135 |
| 608 | 0.141 |
| 609 | 0.061 |
| 610 | 0.031 |
| 611 | 0.031 |
| 612 | 0.012 |
| 613 | 0.133 |
| 614 | 0.097 |
| 615 | 0.053 |
| 616 | 0.019 |
| 617 | 0.012 |
| 618 | 0.012 |
| 619 | 0.029 |
| 620 | 0.016 |
| 621 | 0.026 |
| 622 | 0.036 |
| 623 | 0.010 |
| 624 | 0.026 |
| 625 | >10 |
| 626 | >10 |
| 627 | >10 |
| 628 | >10 |
| 629 | >10 |
| 630 | >10 |
| 631 | >10 |
| 632 | >10 |
| 633 | >10 |
| 634 | >10 |
| 635 | >10 |
| 636 | >10 |
| 637 | >10 |
| 638 | >10 |
| 639 | >10 |
| 640 | >10 |
| 641 | >10 |
| 642 | >10 |

### Example 5. Screening Compounds Using Primary Human Mesenchymal Stem Cells

Representative compounds were screened using primary human mesenchymal stem cells (hMSCs) to determine their ability to induce chondrogenesis (process by which cartilage is developed).

Human Mesenchymal Stem Cell Culture: Primary human mesenchymal stem cells (hMSCs) were purchased from Lonza (Walkersville, Md.) and expanded in Mesenchymal Stem Cell Growth Media (Lonza). Cells between passage 3 and 6 were used for the experiments.

Compound Screening: Each compound was dissolved in DMSO as a 10 mM stock and used to prepare compound source plates. For the 96 well assay, serial dilution (1:3, 6-point dose-response curves from 2700 nM to 10 nM) and compound transfer was performed using the ECHO 550 (Labcyte, Sunnyvale, Calif.) into 96-well clear bottom assay plates (Greiner Bio-One) with appropriate DMSO backfill for a final DMSO concentration of 0.03%. hMSCs were plated at 20,000 cells/well in 250 μL/well Incomplete Chondrogenic Induction Medium (Lonza; DMEM, dexamethasone, ascorbate, insulin-transferrin-selenium [ITS supplement], gentamycin-amphotericin [GA-1000], sodium pyruvate, proline and L-glutamine). TGF-β3 (10 ng/mL) was used as a positive control for differentiation while negative control wells were treated with 75 nL DMSO for normalization and calculating EC₅₀ values. For the 384 well assay, serial dilution (1:3, 8-point dose-response curves from 5000 nM to 2.2 nM) and compound transfer was performed using the ECHO 550 (Labcyte, Sunnyvale, Calif.) into 384-well clear bottom assay plates (Greiner Bio-One) with appropriate DMSO backfill for a final DMSO concentration of 0.03%. hMSCs were plated at 8,000 cells/well in 80 μL/well Incomplete Chondrogenic Induction Medium (Lonza; DMEM, dexamethasone, ascorbate, insulin-transferrin-selenium [ITS supplement], gentamycin-amphotericin [GA-1000], sodium pyruvate, proline and L-glutamine). TGF-β3 (10 ng/mL) was used as a positive control for differentiation while negative control wells were treated with 25 nL DMSO for normalization and calculating EC₅₀ values. Cells were incubated at 37° C. and 5% CO₂ for 6 days. To image chondrogenic nodules, the cells were fixed using 4% formaldehyde (Electron Microscopy Sciences), and stained with 2 μg/mL Rhodamine B (Sigma-Aldrich) and 20 μM Nile Red (Sigma-Aldrich) [Johnson K., et al, A Stem Cell-Based Approach to Cartilage Repair, Science, (2012), 336(6082), 717-721]. The nodules imaged (25 images per well for 96 well plates and 9 images per well for 384 well plates at 10× magnification) by excitation at 531 nm and emission at 625 nm and quantified using the CellInsight CX5 (Thermo Scientific). Area of nodules in each well was normalized to the average of 3 DMSO treated wells on the same plate using Excel (Microsoft Inc.). The normalized averages (fold change over DMSO) of 2 or 3 replicate wells for each compound concentration were calculated. Due to solubility limitations of some of the compounds, curve fitting was incomplete leading to inaccurate EC₅₀ determinations.

Using TGF-β3 as a positive control, the concentration of representative compounds required to induce 50% levels of chondrogenesis is reported. In addition, the maximum activity of each compound and the respective dose that each compound reached maximum chondrogenesis activity is reported.

Table 8 shows the activity of representative compounds of Formulas (I)-(VIII) as provided herein.

TABLE 8

| | | Max. Activity | Conc (nM) |
|---|---|---|---|
| | Conc (nM) | as % | of 50% |
| | of Max. | TGF-β3 | TGF-β3 |
| Compound | activity | activity | activity |
| 2 | 100.00 | 81.50 | NA |
| 5 | 10.00 | 105.94 | 10.00 |
| 6 | 900.00 | 123.63 | 300.00 |
| 7 | 10.00 | 90.66 | NA |
| 10 | 2700.00 | 139.59 | 2700.00 |
| 12 | 300.00 | 75.21 | NA |
| 14 | 30.00 | 89.51 | NA |
| 17 | 2700.00 | 193.68 | 900.00 |
| 23 | 900.00 | 23.35 | NA |
| 25 | 10.00 | 38.95 | NA |
| 26 | 2700.00 | 81.70 | 2700.00 |
| 28 | 30.00 | 68.85 | NA |
| 29 | 10.00 | 74.69 | NA |
| 42 | 30.00 | 25.59 | NA |
| 44 | 100.00 | 44.90 | 100.00 |
| 45 | 30.00 | 54.23 | 30.00 |
| 46 | 30.00 | 43.81 | NA |
| 47 | 300.00 | 82.14 | 300.00 |
| 52 | 100.00 | 52.14 | 30.00 |
| 58 | 100.00 | 92.55 | NA |
| 71 | 30.00 | 63.09 | 30.00 |
| 72 | 300.00 | 72.21 | 100.00 |
| 73 | 300.00 | 80.93 | 30.00 |
| 75 | 2700.00 | 103.95 | 2700.00 |
| 78 | 30.00 | 101.97 | 30.00 |
| 79 | 300.00 | 68.34 | 300.00 |
| 80 | 300.00 | 68.99 | 100.00 |
| 81 | 300.00 | 138.10 | 300.00 |
| 82 | 10.00 | 51.90 | 10.00 |
| 85 | 30.00 | 45.36 | NA |
| 86 | 30.00 | 53.74 | 30.00 |
| 92 | 2700.00 | 128.78 | 2700.00 |
| 98 | 300.00 | 94.43 | 30.00 |
| 101 | 900.00 | 121.01 | 900.00 |
| 102 | 10.00 | 75.71 | 30.00 |
| 106 | 30.00 | 23.57 | NA |
| 107 | 100.00 | 27.40 | NA |
| 119 | 300.00 | 156.71 | 100.00 |
| 120 | 30.00 | 138.52 | 30.00 |
| 121 | 61.73 | 29.30 | NA |
| 122 | 10.00 | 67.82 | 10.00 |
| 123 | 30.00 | 397.03 | 30.00 |
| 124 | 30.00 | 60.96 | NA |
| 126 | 300.00 | 148.02 | 10.00 |
| 127 | 300.00 | 96.85 | NA |
| 128 | 10.00 | 54.67 | NA |
| 129 | 100.00 | 124.50 | 100.00 |
| 130 | 900.00 | 68.09 | NA |
| 131 | 100.00 | 504.45 | 30.00 |
| 132 | 100.00 | 154.45 | 10.00 |
| 133 | 30.00 | 83.83 | 10.00 |
| 134 | 10.00 | 98.66 | NA |
| 135 | 300.00 | 311.36 | 100.00 |
| 136 | 30.00 | 84.58 | 30.00 |
| 137 | 30.00 | 182.87 | 30.00 |
| 138 | 10.00 | 75.63 | 30.00 |
| 139 | 10.00 | 16.68 | NA |
| 141 | 30.00 | 101.95 | 30.00 |
| 142 | 30.00 | 85.77 | 10.00 |
| 143 | 100.00 | 52.25 | NA |
| 144 | 185.19 | 56.69 | 6.86 |
| 145 | 20.58 | 53.57 | 20.58 |
| 146 | 20.58 | 59.30 | 2.29 |
| 147 | 300.00 | 140.43 | 300.00 |
| 148 | 20.58 | 60.38 | 20.58 |
| 149 | 61.73 | 55.53 | 20.58 |
| 150 | 61.73 | 46.95 | NA |
| 153 | 300.00 | 110.41 | 100.00 |
| 154 | 20.58 | 51.83 | 20.58 |
| 155 | 30.00 | 74.94 | 30.00 |
| 156 | 100.00 | 110.06 | 100.00 |
| 157 | 100.00 | 71.04 | 100.00 |
| 158 | 100.00 | 112.45 | 10.00 |
| 159 | 100.00 | 151.67 | 100.00 |
| 160 | 300.00 | 143.19 | 300.00 |
| 161 | 100.00 | 127.78 | 100.00 |
| 162 | 30.00 | 41.80 | NA |
| 163 | 100.00 | 125.28 | 100.00 |
| 164 | 300.00 | 202.71 | 100.00 |
| 165 | 100.00 | 61.91 | 100.00 |
| 166 | 10.00 | 81.84 | 30.00 |
| 167 | 30.00 | 112.83 | 30.00 |
| 168 | 300.00 | 59.89 | 300.00 |
| 169 | 300.00 | 107.78 | 10.00 |
| 170 | 300.00 | 98.17 | 10.00 |
| 171 | 100.00 | 127.12 | 10.00 |
| 172 | 300.00 | 82.80 | NA |
| 173 | 30.00 | 102.25 | 30.00 |
| 174 | 300.00 | 180.88 | 10.00 |
| 175 | 10.00 | 40.08 | NA |
| 176 | 10.00 | 73.98 | 10.00 |
| 177 | 10.00 | 56.78 | NA |
| 178 | 300.00 | 159.03 | 30.00 |
| 179 | 30.00 | 92.95 | 100.00 |
| 180 | 10.00 | 57.77 | 30.00 |
| 181 | 100.00 | 62.92 | NA |
| 182 | 10.00 | 44.51 | NA |
| 183 | 30.00 | 69.96 | 100.00 |
| 184 | 30.00 | 83.81 | 30.00 |
| 185 | 300.00 | 94.60 | 300.00 |
| 186 | 100.00 | 34.37 | NA |
| 187 | 61.73 | 26.05 | NA |
| 189 | 100.00 | 198.15 | 100.00 |
| 190 | 300.00 | 86.34 | NA |
| 191 | 300.00 | 130.24 | 300.00 |
| 193 | 10.00 | 54.36 | 10.00 |
| 194 | 10.00 | 64.61 | 30.00 |
| 195 | 100.00 | 113.50 | 10.00 |
| 196 | 185.19 | 30.96 | NA |
| 198 | 30.00 | 68.59 | 10.00 |
| 200 | 100.00 | 80.49 | 30.00 |
| 201 | 10.00 | 54.83 | 30.00 |
| 202 | 300.00 | 151.62 | 100.00 |
| 203 | 10.00 | 54.09 | 10.00 |
| 204 | 300.00 | 93.85 | NA |
| 206 | 300.00 | 69.83 | 900.00 |
| 207 | 20.58 | 38.34 | NA |
| 208 | 30.00 | 77.29 | NA |
| 209 | 100.00 | 113.89 | 100.00 |
| 210 | 30.00 | 106.19 | 30.00 |
| 211 | 100.00 | 80.09 | NA |
| 212 | 30.00 | 100.12 | 10.00 |
| 213 | 300.00 | 80.62 | NA |
| 214 | 30.00 | 134.58 | 100.00 |
| 215 | 10.00 | 64.62 | 30.00 |
| 216 | 10.00 | 46.94 | NA |
| 218 | 30.00 | 63.82 | NA |
| 219 | 30.00 | 112.69 | 30.00 |
| 220 | 30.00 | 90.42 | NA |
| 221 | 10.00 | 69.65 | 30.00 |
| 222 | 2700.00 | 149.58 | 100.00 |
| 223 | 100.00 | 70.15 | 10.00 |
| 224 | 30.00 | 73.66 | 30.00 |
| 226 | 100.00 | 69.27 | 30.00 |
| 227 | 100.00 | 47.21 | NA |
| 228 | 100.00 | 69.07 | 300.00 |
| 229 | 100.00 | 148.85 | 100.00 |
| 230 | 100.00 | 111.47 | 100.00 |
| 231 | 10.00 | 73.57 | 10.00 |
| 232 | 300.00 | 143.65 | 30.00 |
| 233 | 10.00 | 57.99 | NA |
| 234 | 30.00 | 101.69 | 30.00 |
| 235 | 100.00 | 109.62 | 100.00 |
| 236 | 300.00 | 90.17 | 30.00 |
| 237 | 30.00 | 72.03 | 30.00 |
| 238 | 30.00 | 76.17 | 30.00 |
| 239 | 30.00 | 75.32 | 30.00 |
| 240 | 100.00 | 64.24 | 100.00 |
| 242 | 300.00 | 155.71 | 10.00 |
| 243 | 100.00 | 166.67 | 100.00 |
| 244 | 30.00 | 202.56 | 30.00 |
| 245 | 30.00 | 72.64 | 30.00 |
| 246 | 30.00 | 42.02 | NA |
| 247 | 20.58 | 58.38 | 20.58 |
| 249 | 30.00 | 67.73 | 30.00 |
| 250 | 100.00 | 167.12 | 100.00 |
| 251 | 300.00 | 105.29 | 300.00 |
| 252 | 100.00 | 91.27 | NA |
| 253 | 100.00 | 81.26 | NA |
| 254 | 30.00 | 41.58 | NA |
| 255 | 30.00 | 69.78 | NA |
| 256 | 10.00 | 53.30 | NA |
| 257 | 10.00 | 68.35 | NA |
| 258 | 10.00 | 65.63 | 10.00 |
| 259 | 30.00 | 74.59 | NA |
| 260 | 30.00 | 145.63 | 30.00 |
| 261 | 100.00 | 142.24 | 10.00 |
| 262 | 300.00 | 120.09 | 30.00 |
| 263 | 300.00 | 40.87 | NA |
| 265 | 30.00 | 84.72 | 10.00 |
| 266 | 10.00 | 60.59 | 10.00 |
| 267 | 10.00 | 60.19 | 30.00 |
| 268 | 10.00 | 67.88 | 30.00 |
| 269 | 100.00 | 102.25 | 10.00 |
| 270 | 10.00 | 61.98 | 10.00 |
| 271 | 10.00 | 87.93 | 10.00 |
| 272 | 10.00 | 163.06 | 2700.00 |
| 273 | 30.00 | 258.86 | 10.00 |
| 274 | 100.00 | 161.88 | 100.00 |
| 275 | 10.00 | 72.56 | 10.00 |
| 276 | 100.00 | 79.43 | 30.00 |
| 277 | 100.00 | 230.76 | 100.00 |
| 278 | 300.00 | 129.40 | 300.00 |
| 279 | 10.00 | 64.71 | 10.00 |
| 280 | 10.00 | 71.42 | 10.00 |
| 281 | 10.00 | 82.05 | NA |
| 282 | 30.00 | 85.12 | 30.00 |
| 283 | 30.00 | 82.91 | 30.00 |
| 284 | 2700.00 | 96.85 | 300.00 |
| 285 | 61.73 | 35.60 | NA |
| 286 | 20.58 | 149.15 | 20.58 |
| 288 | 100.00 | 81.83 | 30.00 |
| 289 | 10.00 | 92.78 | 10.00 |
| 291 | 30.00 | 70.00 | 30.00 |
| 292 | 100.00 | 118.06 | 100.00 |
| 293 | 30.00 | 73.13 | 30.00 |
| 294 | 100.00 | 113.70 | 30.00 |
| 295 | 10.00 | 50.58 | 30.00 |
| 296 | 300.00 | 59.20 | 30.00 |
| 297 | 30.00 | 50.46 | 100.00 |
| 298 | 10.00 | 20.31 | NA |
| 300 | 100.00 | 35.62 | NA |
| 301 | 30.00 | 44.84 | NA |
| 302 | 10.00 | 16.19 | NA |
| 303 | 100.00 | 33.55 | NA |
| 304 | 100.00 | 166.66 | 100.00 |
| 305 | 100.00 | 112.53 | 10.00 |
| 306 | 30.00 | 50.78 | 30.00 |
| 307 | 300.00 | 42.25 | NA |
| 308 | 300.00 | 42.05 | NA |
| 309 | 10.00 | 192.93 | 10.00 |
| 310 | 10.00 | 109.95 | 10.00 |
| 311 | 10.00 | 59.96 | 30.00 |
| 312 | 100.00 | 48.05 | NA |
| 313 | 30.00 | 108.39 | 10.00 |
| 314 | 30.00 | 96.23 | 30.00 |
| 315 | 30.00 | 98.45 | 30.00 |
| 316 | 10.00 | 61.18 | 10.00 |
| 317 | 300.00 | 78.39 | 100.00 |
| 318 | 300.00 | 112.64 | 900.00 |
| 319 | 10.00 | 23.78 | NA |
| 320 | 300.00 | 23.53 | NA |
| 321 | 300.00 | 85.98 | 30.00 |
| 322 | 100.00 | 27.61 | NA |
| 323 | 300.00 | 117.15 | 10.00 |
| 324 | 300.00 | 15.54 | NA |
| 325 | 10.00 | 32.35 | NA |
| 326 | 300.00 | 36.29 | NA |
| 327 | 30.00 | 54.34 | 30.00 |
| 328 | 30.00 | 78.06 | 30.00 |
| 329 | 10.00 | 52.09 | 10.00 |
| 330 | 10.00 | 24.77 | NA |
| 331 | 30.00 | 70.81 | 10.00 |
| 332 | 10.00 | 75.80 | 10.00 |
| 333 | 10.00 | 81.62 | 10.00 |
| 334 | 100.00 | 37.32 | NA |
| 335 | 30.00 | 270.04 | 30.00 |
| 336 | 30.00 | 60.86 | 30.00 |
| 337 | 10.00 | 50.58 | 30.00 |
| 338 | 10.00 | 69.41 | 10.00 |
| 339 | 100.00 | 51.13 | 100.00 |
| 340 | 300.00 | 89.16 | 10.00 |
| 341 | 100.00 | 58.20 | 100.00 |
| 342 | 10.00 | 16.72 | NA |
| 343 | 100.00 | 76.37 | 100.00 |
| 344 | 30.00 | 59.25 | 100.00 |
| 345 | 300.00 | 17.37 | NA |
| 346 | 100.00 | 148.84 | 10.00 |
| 347 | 300.00 | 178.02 | 100.00 |
| 348 | 10.00 | 671.43 | 10.00 |
| 349 | 100.00 | 34.48 | NA |
| 350 | 10.00 | 200.61 | 10.00 |
| 351 | 10.00 | 52.76 | 10.00 |
| 352 | 10.00 | 43.53 | NA |
| 353 | 30.00 | 37.32 | NA |
| 354 | 100.00 | 307.85 | 100.00 |
| 355 | 300.00 | 41.31 | NA |
| 356 | 10.00 | 45.46 | NA |
| 357 | 300.00 | 19.45 | NA |
| 358 | 10.00 | 68.52 | 10.00 |
| 359 | 10.00 | 225.83 | 10.00 |
| 360 | 100.00 | 41.90 | NA |
| 361 | 300.00 | 45.50 | NA |
| 362 | 10.00 | 134.56 | 10.00 |
| 363 | 10.00 | 77.01 | 10.00 |
| 364 | 100.00 | 47.23 | NA |
| 365 | 30.00 | 28.72 | NA |
| 366 | 10.00 | 91.84 | 10.00 |
| 367 | 10.00 | 67.56 | 10.00 |
| 368 | 10.00 | 35.89 | NA |
| 369 | 300.00 | 16.70 | NA |
| 370 | 300.00 | 20.26 | NA |
| 371 | 300.00 | 23.29 | NA |
| 372 | 300.00 | 43.91 | NA |
| 373 | 300.00 | 24.15 | NA |
| 374 | 300.00 | 49.61 | NA |
| 375 | 100.00 | 65.19 | 30.00 |
| 376 | 300.00 | 56.47 | 300.00 |
| 377 | 100.00 | 76.76 | 100.00 |
| 378 | 30.00 | 53.50 | 30.00 |
| 379 | 100.00 | 31.53 | NA |
| 380 | 100.00 | 11.95 | NA |
| 381 | 30.00 | 52.05 | 10.00 |
| 382 | 30.00 | 26.87 | NA |
| 383 | 100.00 | 124.27 | 100.00 |
| 384 | 30.00 | 50.30 | 30.00 |
| 385 | 300.00 | 32.14 | NA |
| 386 | 100.00 | 70.43 | 10.00 |
| 387 | 30.00 | 95.48 | 30.00 |
| 388 | 30.00 | 70.15 | 30.00 |
| 389 | 100.00 | 71.37 | 30.00 |
| 390 | 100.00 | 44.44 | NA |
| 391 | 300.00 | 33.63 | NA |
| 392 | 30.00 | 30.42 | NA |
| 393 | 30.00 | 26.71 | NA |
| 394 | 30.00 | 43.48 | NA |
| 395 | 300.00 | 113.87 | 30.00 |
| 396 | 100.00 | 67.18 | 100.00 |
| 397 | 30.00 | 191.09 | 10.00 |
| 398 | 300.00 | 17.82 | NA |
| 399 | 30.00 | 51.72 | 30.00 |
| 400 | 300.00 | 33.36 | NA |
| 401 | 100.00 | 29.21 | NA |
| 402 | 300.00 | 36.55 | NA |
| 403 | 10.00 | 97.11 | 10.00 |
| 404 | 300.00 | 39.60 | NA |
| 405 | 100.00 | 17.28 | NA |
| 406 | 10.00 | 194.10 | 10.00 |
| 407 | 100.00 | 46.08 | NA |
| 408 | 30.00 | 8.03 | NA |
| 409 | 30.00 | 81.89 | 10.00 |
| 410 | 30.00 | 341.50 | 10.00 |
| 411 | 10.00 | 11.03 | NA |
| 412 | 10.00 | 62.01 | 30.00 |
| 413 | 30.00 | 27.60 | NA |
| 414 | 10.00 | 65.88 | 10.00 |
| 415 | 300.00 | 160.71 | 300.00 |
| 416 | 100.00 | 61.37 | 10.00 |
| 417 | 30.00 | 35.50 | NA |
| 418 | 300.00 | 16.91 | NA |
| 419 | 100.00 | 61.23 | 30.00 |
| 420 | 10.00 | 34.10 | NA |
| 421 | 300.00 | 30.79 | NA |
| 422 | 10.00 | 49.91 | NA |
| 423 | 100.00 | 40.37 | NA |
| 424 | 300.00 | 7.07 | NA |
| 425 | 100.00 | 58.81 | 10.00 |
| 426 | 30.00 | 58.48 | 30.00 |
| 427 | 30.00 | 17.69 | NA |
| 428 | 30.00 | 213.44 | 10.00 |
| 429 | 30.00 | 30.25 | NA |
| 430 | 30.00 | 50.32 | 30.00 |
| 431 | 2700.00 | 155.01 | 30.00 |
| 432 | 100.00 | 28.47 | NA |
| 433 | 10.00 | 59.37 | 30.00 |
| 434 | 10.00 | 119.14 | 10.00 |
| 435 | 30.00 | 69.86 | 30.00 |
| 436 | 10.00 | 21.83 | NA |
| 437 | 300.00 | 151.58 | 10.00 |
| 438 | 10.00 | 531.63 | 10.00 |
| 439 | 10.00 | 317.16 | 10.00 |
| 440 | 10.00 | 79.94 | 10.00 |
| 441 | 300.00 | 100.58 | 300.00 |
| 442 | 30.00 | 119.04 | 30.00 |
| 443 | 10.00 | 36.97 | NA |
| 444 | 10.00 | 97.58 | 10.00 |
| 445 | 10.00 | 45.33 | NA |
| 446 | 300.00 | 134.44 | 300.00 |
| 447 | 30.00 | 134.32 | 100.00 |
| 448 | 100.00 | 122.15 | 100.00 |
| 450 | 10.00 | 50.80 | 10.00 |
| 451 | 100.00 | 39.72 | NA |
| 452 | 30.00 | 44.64 | NA |
| 453 | 100.00 | 42.68 | NA |
| 454 | 300.00 | 51.70 | 300.00 |
| 455 | 30.00 | 129.63 | 30.00 |
| 456 | 100.00 | 93.54 | 30.00 |
| 457 | 30.00 | 243.99 | 10.00 |
| 458 | 100.00 | 68.63 | 100.00 |
| 459 | 30.00 | 54.49 | 30.00 |
| 460 | 300.00 | 42.48 | NA |
| 461 | 300.00 | 42.39 | NA |
| 462 | 30.00 | 21.83 | NA |
| 463 | 300.00 | 33.61 | NA |
| 464 | 300.00 | 204.88 | 10.00 |
| 465 | 30.00 | 220.08 | 10.00 |
| 466 | 300.00 | 24.71 | NA |
| 467 | 100.00 | 159.84 | 30.00 |
| 468 | 100.00 | 37.61 | NA |
| 469 | 100.00 | 48.11 | NA |
| 470 | 30.00 | 50.89 | 30.00 |
| 471 | 100.00 | 50.22 | 100.00 |
| 472 | 10.00 | 49.80 | NA |
| 473 | 300.00 | 70.02 | 30.00 |
| 474 | 10.00 | 36.43 | NA |
| 475 | 300.00 | 21.03 | NA |
| 476 | 30.00 | 61.59 | 30.00 |
| 477 | 10.00 | 49.47 | NA |
| 478 | 30.00 | 88.20 | 10.00 |
| 479 | 300.00 | 30.77 | NA |
| 480 | 300.00 | 30.28 | NA |
| 481 | 100.00 | 30.27 | NA |
| 482 | 100.00 | 37.84 | NA |
| 483 | 100.00 | 60.82 | 10.00 |
| 484 | 300.00 | 42.80 | NA |
| 485 | 100.00 | 288.64 | 100.00 |
| 486 | 100.00 | 34.64 | NA |
| 487 | 10.00 | 67.03 | 10.00 |
| 488 | 10.00 | 61.82 | 10.00 |
| 489 | 300.00 | 149.76 | 300.00 |
| 490 | 30.00 | 101.29 | 30.00 |
| 491 | 30.00 | 13.94 | NA |
| 492 | 300.00 | 31.04 | NA |
| 493 | 300.00 | 198.09 | 100.00 |
| 494 | 100.00 | 23.56 | NA |
| 494 | 300.00 | 50.74 | 300.00 |
| 496 | 300.00 | 70.79 | 10.00 |
| 497 | 300.00 | 20.08 | NA |
| 498 | 300.00 | 60.11 | 300.00 |
| 499 | 100.00 | 122.47 | 30.00 |
| 500 | 100.00 | 21.19 | NA |
| 501 | 10.00 | 101.61 | 10.00 |
| 502 | 10.00 | 25.30 | NA |
| 503 | 100.00 | 72.07 | 30.00 |
| 504 | 300.00 | 35.53 | NA |
| 505 | 100.00 | 40.25 | NA |
| 506 | 10.00 | 81.39 | 10.00 |
| 507 | 100.00 | 82.21 | 100.00 |
| 508 | 10.00 | 57.35 | 10.00 |
| 509 | 10.00 | 59.04 | 10.00 |
| 510 | 300.00 | 51.44 | 300.00 |
| 511 | 100.00 | 180.26 | 100.00 |
| 512 | 10.00 | 162.22 | 10.00 |
| 513 | 30.00 | 87.33 | 10.00 |
| 514 | 30.00 | 48.45 | NA |
| 515 | 10.00 | 66.42 | 10.00 |
| 516 | 10.00 | 181.12 | 10.00 |
| 517 | 100.00 | 29.35 | NA |
| 518 | 10.00 | 36.72 | NA |
| 519 | 30.00 | 59.64 | 30.00 |
| 520 | 10.00 | 79.53 | 30.00 |
| 521 | 10.00 | 110.65 | 10.00 |
| 522 | 300.00 | 52.03 | 300.00 |
| 523 | 300.00 | 45.54 | NA |
| 524 | 300.00 | 24.16 | NA |
| 525 | 10.00 | 33.13 | NA |
| 526 | 10.00 | 82.89 | 10.00 |
| 527 | 100.00 | 14.51 | NA |
| 528 | 300.00 | 51.59 | 300.00 |
| 529 | 10.00 | 15.49 | NA |
| 530 | 10.00 | 109.70 | 10.00 |
| 531 | 100.00 | 58.03 | 100.00 |
| 532 | 300.00 | 61.21 | 300.00 |
| 533 | 100.00 | 42.79 | NA |
| 534 | 10.00 | 52.33 | 30.00 |
| 535 | 10.00 | 22.73 | NA |
| 536 | 10.00 | 50.08 | 10.00 |
| 537 | 300.00 | 20.61 | NA |
| 538 | 30.00 | 106.38 | 10.00 |
| 541 | 10.00 | 25.44 | NA |
| 545 | 10.00 | 27.11 | NA |
| 572 | 30.00 | 27.60 | NA |
| 575 | 300.00 | 18.63 | NA |
| 580 | 2700.00 | 24.91 | NA |
| 591 | 30.00 | 22.26 | NA |
| 595 | 30.00 | 25.69 | NA |
| 603 | 2700.00 | 67.97 | 2700.00 |
| 617 | 5000.00 | 231.44 | 5000.00 |
| 618 | 1700.00 | 558.12 | 200.00 |
| 619 | 100.00 | 224.83 | 50.00 |
| 620 | 100.00 | 185.47 | 100.00 |
| 621 | 10.00 | 180.89 | 10.00 |
| 622 | 30.00 | 69.41 | 30.00 |
| 623 | 10.00 | 73.44 | 30.00 |

NA = Not applicable

### Example 6. Catabolism Prevention Assay

Representative compounds were screened using primary human chondrocytes to determine their ability to protect against catabolic breakdown by measuring levels of cartilage degradative protease enzymes-matrix metalloproteinases (MMPs).

Human Chondrocyte Cell Culture: Primary human chondrocytes were purchased from Lonza (Walkersville, Md.) and expanded in Chondrocyte Growth Media (CGM, Lonza). Cells between passage 2 and 5 were used for the experiments.

Chondrocyte Protection assay: Chondrocytes were plated at 300,000 cells/well in 2 mL/well Chondrocyte Growth Medium (Lonza; 500 mL Basal Medium, 25 mL FBS, 0.5 mL GA-1000, 1 mL R3-IGF, 2.5 mL bFGF, 0.5 mL Transferrin, and 1 mL Insulin). Cells were incubated at 37° C. and 5% CO₂ overnight. Media was replaced with 3 mL of Chondrocyte Growth Medium. Each compound was dissolved in DMSO as a 10 mM stock. Serial dilutions (300 nM, 100 nM, 30 nM) and compound transfer was performed manually with appropriate DMSO backfill for a final DMSO concentration of 0.1%. After 2 hours of incubation at 37° C. and 5% CO₂, IL1-β (Peprotech, Rocky Hill, N.J.) was added to a final concentration of 10 ng/mL. 1 well was left unstimulated. Cells were incubated at 37° C. and 5% CO₂ for 24 hr. Media was aspirated and washed twice with Phosphate Buffered Saline (ThermoFisher, Waltham, Mass.) then cells were frozen in the plate at −80° C. Total RNA was isolated from the frozen chondrocytes using the RNeasy Mini Kit (Qiagen, Valencia, Calif.). RNA concentrations were measured using the Cytation3 (Biotek Instruments Inc., Winooski, Vt.). cDNA was synthesized from 1 g of RNA using the QuantiTect Reverse Transcription kit (Qiagen, Valencia, Vt.) and a thermal cycler (Bio-Rad, Hercules, Calif.). qRT-PCR was performed with QuantiTect SYBR Green PCR Kit (Qiagen, Valencia, Calif.) and gene-specific primers for MMP-1, MMP-3 and MMP-13, using CFX384 (Bio-Rad, Hercules, Calif.). Transcripts were quantitated by comparative Ct method and normalized to endogenous controls, Glyceraldehyde 3-phosphate dehydrogenase (GAPDH) and 18S. Expression values were normalized to the expression of the unstimulated well on the same plate using Excel (Microsoft Inc.). The normalized averages (fold change over unstimulated) for each compound concentration were calculated.

Table 9 shows the expression values for MMP-1, MMP-3, MMP-13 of representative compounds of Formulas (I)-(VIII) as provided herein.

TABLE 9

| | Conc (nM) of Max. activity | | |
|---|---|---|---|
| Compound | MMP-1 | MMP-3 | MMP-13 |
| 23 | 18.25 | NA | NA |
| 25 | 40.33 | NA | NA |
| 26 | NA | NA | 59.49 |
| 42 | NA | NA | NA |
| 46 | NA | 58.60 | NA |
| 52 | NA | NA | NA |
| 106 | 76.01 | 65.63 | 83.32 |
| 107 | NA | NA | 63.03 |
| 123 | 13.78 | 37.44 | 44.97 |
| 219 | 92.23 | 80.34 | 63.39 |
| 298 | 86.58 | 46.89 | 50.55 |
| 541 | 90.64 | NA | 63.55 |
| 545 | 41.79 | 46.43 | NA |
| 572 | 77.86 | 58.87 | 75.49 |
| 575 | 19.31 | NA | 65.25 |
| 580 | 84.92 | 84.26 | 87.48 |
| 591 | 76.26 | 75.64 | 89.81 |
| 595 | 76.53 | 70.04 | 81.98 |
| 603 | 95.71 | 98.35 | 73.09 |
| 621 | 66.60 | NA | NA |
| 622 | NA | NA | NA |
| 623 | 48.59 | NA | NA |

NA = No inhibition

Chondrocyte Protection assay compound screening: Each compound was dissolved in DMSO as a 10 mM stock and used to prepare compound source plates. For the assay, serial dilution (1:3, 11-point dose-response curves from 3 μM to 0.05 nM) and compound transfer was performed using the ECHO 550 (Labcyte, Sunnyvale, Calif.) into 384-well assay plates (Greiner Bio-One) with appropriate DMSO backfill for a final DMSO concentration of 0.1%. Chondrocytes were plated at 8,000 cells/well in 40 μL/well Chondrocyte Differentiation Medium (Lonza; 500 mL Basal Medium, 25 mL FBS, 0.5 mL GA-1000, 1 mL R3-IGF, 2.5 mL bFGF, 0.5 mL Transferrin, and 1 mL Insulin). After 2 hours of incubation at 37° C. and 5% CO₂, IL1-β (Peprotech, Rocky Hill, N.J.) was added to a final concentration of 10 ng/mL. 11 wells in 1 row were left unstimulated for normalization and calculating EC₅₀ values. Cells were incubated at 37° C. and 5% CO₂ overnight. Assay plates are spun, 25 μl of supernatant media was collected using (S2 pipettor, Apricot Designs). Cells were lysed using 10 μL of (Cell Titer-Glo, Promega). Luminescent signal detected using an En Vision Multilabel reader (Perkin Elmer). MMP-3 levels were measured in supernatant. Supernatant diluted 1:7 in DMEM. MMP-3 levels in the supernatant were measured using MMP-3 LANCE Ultra detection kit (Perkin Elmer). Briefly, 5 μL of diluted supernatant was transferred to low-volume 384 well plates using (S2 pipettor, Apricot Designs). A standard curve was prepared using MMP-3 analyte (range 300,000 μg/mL to 1 μg/mL). 1.7 μL of 8× mix of Eu-labelled anti-MMP-3 antibody and ULight labelled anti-MMP-3 antibody was added to the plates using Mantis Liquid handler (Formulatrix). Plates were incubated overnight at room temperature. LANCE signal was detected using an EnVision Multilabel reader (Perkin Elmer), equipped with TR-FRET using excitation wavelength of 320 nm and two emission wavelengths of 615 nm and 665 nm. Data was analyzed according to the kit protocol, using the ratio of 615 nm/665 nm and the standard curve values were used to calculate the amount of MMP3 in the lysates (in pg/ml). The normalized averages (fold change over IL1-β stimulated) for each compound concentration were calculated. EC₅₀ and maximum % inhibition values were calculated using the dose-response nonlinear regression curve-fit in Dotmatics.

Table 10 shows the activity of representative compounds of Formulas (I)-(VIII) as provided herein.

TABLE 10

| | | Maximum % |
|---|---|---|
| Compound | EC₅₀ (μM) | Inhibition |
| 1 | 0.0389 | 100.0 |
| 2 | 0.0004 | 58.2 |
| 3 | 0.8107 | 23.7 |
| 4 | 2.9970 | 99.9 |
| 6 | 0.0022 | 42.1 |
| 8 | 0.3421 | 76.0 |
| 9 | 2.9970 | 65.6 |
| 10 | 0.0015 | 46.9 |
| 11 | 2.9970 | 80.0 |
| 12 | 0.2803 | 88.7 |
| 13 | 3.0000 | 71.0 |
| 14 | 0.3387 | 71.9 |
| 15 | 0.0765 | 100.0 |
| 16 | 0.0087 | 99.5 |
| 17 | 0.0037 | 64.7 |
| 18 | 1.0530 | 25.5 |
| 19 | 2.9970 | 31.1 |
| 20 | 0.0002 | 72.6 |
| 21 | 2.9970 | 92.6 |
| 22 | 0.0178 | 64.6 |
| 23 | 2.9970 | 51.7 |
| 24 | 3.0000 | 74.1 |
| 25 | 2.9970 | 91.9 |
| 26 | 0.0014 | 64.6 |
| 27 | 3.0000 | 88.5 |
| 30 | 2.9970 | 3.4 |
| 31 | 2.9970 | 2.4 |
| 32 | 0.0002 | 77.8 |
| 34 | 2.9970 | 12.9 |
| 35 | 2.9970 | 99.3 |
| 36 | 1.6120 | ND |
| 37 | 2.9970 | 55.1 |
| 38 | 2.9970 | 97.5 |
| 39 | 0.0478 | 60.8 |
| 43 | 2.9970 | 41.9 |
| 46 | 1.0535 | 100.0 |
| 48 | 2.2683 | 100.0 |
| 49 | 0.0035 | 66.1 |
| 50 | 2.9970 | 44.4 |
| 51 | 2.9970 | 48.1 |
| 53 | 3.0000 | 74.9 |
| 57 | 3.0000 | 54.9 |
| 58 | 0.0108 | 61.1 |
| 59 | 0.0016 | 99.2 |
| 61 | 0.0022 | 69.2 |
| 65 | 2.9970 | 98.1 |
| 66 | 0.0049 | 62.6 |
| 67 | 2.9970 | 48.7 |
| 68 | 2.9970 | 26.6 |
| 69 | 0.0697 | 12.3 |
| 74 | 0.0023 | 62.7 |
| 75 | 0.0080 | 95.1 |
| 76 | 0.0004 | 97.9 |
| 77 | 2.9970 | 84.8 |
| 83 | 0.3034 | ND |
| 87 | 2.2248 | 100.0 |
| 88 | 2.9970 | 100.0 |
| 89 | 0.0055 | 79.8 |
| 90 | 0.0067 | 29.7 |
| 96 | 0.1797 | 100.0 |
| 99 | 2.9970 | 39.6 |
| 100 | 0.6155 | 8.6 |
| 101 | 2.9970 | 45.7 |
| 102 | 0.0002 | 26.3 |
| 103 | 0.3071 | 36.3 |
| 104 | 0.0071 | 72.8 |
| 105 | 2.7374 | 53.1 |
| 106 | 0.8179 | 100.0 |
| 107 | 2.9970 | 22.9 |
| 108 | 2.9970 | 32.1 |
| 109 | 1.1565 | 100.0 |
| 110 | 1.0796 | 87.2 |
| 111 | 0.0126 | 76.2 |
| 112 | 1.1986 | 88.3 |
| 113 | 2.9970 | 57.0 |
| 114 | 2.9970 | 62.8 |
| 116 | 0.0130 | 61.2 |
| 117 | 0.0442 | 94.4 |
| 118 | 0.1378 | 21.1 |
| 125 | 2.9970 | ND |
| 130 | 0.0406 | 60.5 |
| 140 | 0.0008 | 76.4 |
| 152 | 1.7070 | 31.8 |
| 173 | 2.9970 | 90.2 |
| 188 | 1.0996 | 100.0 |
| 189 | 0.1107 | 100.0 |
| 192 | 0.0019 | 95.5 |
| 199 | 0.0024 | 80.9 |
| 205 | 0.0208 | 61.5 |
| 208 | 0.0205 | 39.0 |
| 219 | 0.0355 | 87.3 |
| 220 | 0.0136 | 100.0 |
| 222 | 1.1471 | 64.9 |
| 225 | 0.845 | 89.2 |
| 241 | 0.0001 | 87.1 |
| 248 | 2.9970 | 40.7 |
| 260 | 2.9970 | ND |
| 264 | 2.9970 | 81.0 |
| 275 | 2.9970 | 58.4 |
| 284 | 2.9970 | 32.9 |
| 287 | 0.0128 | 74.2 |
| 290 | 0.6042 | ND |
| 298 | 2.9970 | 23.2 |
| 299 | 0.1702 | 100.0 |
| 341 | 0.0011 | 58.2 |
| 367 | 2.9970 | 75.7 |
| 394 | 0.0179 | 90.4 |
| 431 | 0.0067 | 100.0 |
| 449 | 0.3188 | 100.0 |
| 462 | 1.4680 | 99.9 |
| 490 | 0.3289 | 80.2 |
| 533 | 2.9970 | 40.8 |
| 539 | 0.0328 | 42.3 |
| 540 | 0.0474 | 80.7 |
| 541 | 3.0000 | 95.6 |
| 542 | 0.0095 | 47.6 |
| 543 | 0.3059 | 81.8 |
| 544 | 1.0073 | 63.5 |
| 546 | 0.0004 | 97.3 |
| 547 | 0.8979 | 52.1 |
| 548 | 2.9970 | 62.4 |
| 549 | 2.9970 | 69.0 |
| 550 | 2.9970 | 66.9 |
| 551 | 1.0550 | 100.0 |
| 552 | 0.6971 | 83.2 |
| 553 | 0.3044 | 52.3 |
| 554 | 2.9970 | 53.7 |
| 555 | 2.9970 | 85.1 |
| 556 | 0.1537 | 100.0 |
| 557 | 1.6340 | 50.5 |
| 558 | 2.9970 | 46.9 |
| 559 | 0.0191 | 41.2 |
| 560 | 2.9970 | 63.6 |
| 562 | 0.0881 | 34.8 |
| 563 | 0.0573 | 72.4 |
| 564 | 0.0714 | 0.0 |
| 565 | 0.0122 | 76.6 |
| 566 | 0.0115 | 43.8 |
| 567 | 1.3710 | 69.8 |
| 568 | 2.9970 | 42.7 |
| 569 | 2.9970 | 0.2 |
| 570 | 2.9970 | 80.6 |
| 571 | 0.0350 | 51.0 |
| 572 | 1.189 | 96.5 |
| 573 | 2.9970 | 70.0 |
| 574 | 1.9190 | 59.5 |
| 575 | 0.0758 | 41.5 |
| 576 | 2.9970 | 62.6 |
| 578 | 2.9970 | 37.6 |
| 579 | 0.4920 | 91.0 |
| 580 | 2.9970 | 59.4 |
| 582 | 2.9970 | 52.2 |
| 583 | 2.9970 | 67.5 |
| 584 | 0.0715 | 38.6 |
| 585 | 1.4320 | 77.9 |
| 586 | 0.0046 | 41.7 |
| 587 | 0.0187 | ND |
| 588 | 0.0105 | 74.7 |
| 589 | 2.9970 | 43.5 |
| 590 | 2.9970 | 33.2 |
| 591 | 0.423 | 100.0 |
| 592 | 2.9970 | 29.7 |
| 593 | 0.0004 | 62.9 |
| 594 | 0.1434 | 35.3 |
| 595 | 0.0027 | 86.2 |
| 596 | 0.7976 | 39.9 |
| 597 | 0.0029 | 3.7 |
| 599 | 2.9970 | 66.4 |
| 601 | 2.9970 | 91.6 |
| 602 | 0.8974 | 99.5 |
| 603 | 3.0000 | 55.5 |
| 604 | 0.0401 | 57.4 |
| 605 | 2.9970 | 59.9 |
| 606 | 2.9970 | 76.1 |
| 607 | 0.4167 | 79.7 |
| 608 | 2.9970 | 18.3 |
| 609 | 0.1543 | 50.2 |
| 610 | 2.9970 | 30.4 |
| 611 | 0.0087 | 68.3 |
| 612 | 2.9970 | 38.6 |
| 613 | 0.0466 | 51.4 |
| 614 | 2.9970 | 41.1 |
| 616 | 2.9970 | 48.3 |
| 619 | 3.0000 | 71.5 |
| 620 | 0.0374 | 37.1 |
| 621 | 3.777 | 46.5 |
| 622 | 0.010 | 83.8 |
| 623 | 9.985 | 89.5 |
| 625 | 2.9970 | 70.9 |
| 627 | 2.9970 | 90.7 |
| 628 | 2.9970 | 54.3 |
| 629 | 0.0002 | 70.0 |
| 630 | 0.0051 | 67.0 |
| 631 | 0.0067 | 72.0 |
| 632 | 0.1233 | 72.2 |
| 634 | 0.0461 | 83.8 |
| 635 | 0.0040 | 78.5 |
| 636 | 0.2977 | 64.5 |
| 637 | 2.9970 | 39.1 |
| 639 | 2.9970 | 43.7 |
| 641 | 0.0358 | 48.4 |
| 642 | 2.9970 | 29.5 |

ND = No data

1. A method of treating osteoarthritis in a subject, the method comprising administering a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof, to a subject identified as having an elevated level of a biomarker associated with inflammation.

2. A method of selecting a subject for treatment, the method comprising:
(a) performing a diagnostic test on the subject to confirm osteoarthritis; and
(b) selecting the subject for treatment, wherein the treatment includes administration of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

3. The method of claim 2, wherein the diagnostic test comprises at least one of joint aspiration, X-ray, and MRI.

4. A method of selecting a subject for treatment, the method comprising selecting a subject identified as having an elevated level of a biomarker associated with inflammation for treatment with a therapeutically effective amount of a single compound, wherein the single compound is a dual DYRK1A/CLK2 and/or CLK3 inhibitor, which inhibits DYRK1A at an IC₅₀ value of less than 100 nM and CLK2 and/or CLK3 at an IC₅₀ value of less than 100 nM, or a pharmaceutically acceptable salt or solvate thereof.

5.-8. (canceled)

9. A method of modifying the progression of osteoarthritis in a subject in need thereof, the method comprising:
(a) identifying a subject, wherein the subject has an elevated level of Wnt pathway activity in a sample from the subject, as compared to a reference level; and
(b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

10. A method of treating osteoarthritis in a subject, the method comprising administering a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof, to a subject identified as having an elevated level of Wnt pathway activity in a sample from the subject, as compared to a reference level.

11. A method of selecting a subject for treatment, the method comprising:
(a) detecting an elevated level of Wnt pathway activity in a sample from the subject, as compared to a reference level; and
(b) selecting the subject for treatment, wherein the treatment includes administration of a single compound, wherein the single compound inhibits CLK2 and/or CLK3 at an IC₅₀ value of less than 100 nM and DYRK1A at an IC₅₀ value of less than 100 nM, or a pharmaceutically acceptable salt or solvate thereof.

12. A method of selecting a subject for treatment, the method comprising selecting a subject identified as having an elevated level of Wnt pathway activity in a sample from the subject as compared to a reference level, and treating the subject with a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

13. A method of inducing chondrogenesis in a subject in need thereof, the method comprising
(a) detecting an elevated level of Wnt pathway activity in a sample from the subject, as compared to a reference level; and
(b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof, wherein the subject exhibits chondrogenesis.

14. A method of treating osteoarthritis in a subject in need thereof, the method comprising:
(a) detecting an elevated level of a biomarker associated with inflammation in a sample of the subject; and
(b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

15. A method of treating osteoarthritis in a subject in need thereof, the method comprising:
(a) identifying a subject, wherein the subject is identified when a sample of the subject has an elevated level of a biomarker associated with inflammation; and
(b) administering to the subject a therapeutically effective amount of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

16. A method of selecting a subject for treatment, the method comprising:
(a) detecting an elevated level of a biomarker associated with inflammation in a sample of the subject; and
(b) selecting the subject for treatment, wherein the treatment includes administration of a single compound, wherein the single compound is a dual DYRK1A/CLK2 and/or CLK3 inhibitor, which inhibits CLK2 and/or CLK3 at an IC₅₀ value of less than 100 nM and DYRK1A at an IC₅₀ value of less than 100 nM, or a pharmaceutically acceptable salt or solvate thereof.

17.-41. (canceled)

42. The method of claim 1, wherein the method induces chondrogenesis.

43. The method of claim 1, wherein the method induces chondrocyte differentiation.

44. The method of claim 1, wherein the method increases chondrocyte function.

45. The method of claim 1, wherein the method prevents cartilage breakdown.

46. The method of claim 1, wherein the method prevents chondrocyte catabolic effects.

47. The method of claim 1, wherein the method induces cartilage growth.

48. (canceled)

49. The method of claim 1, wherein chondrogenesis is increased compared to the chondrogenesis observed prior to administration of the single compound or a pharmaceutically acceptable salt of solvate thereof.

50.-59. (canceled)

60. The method of claim 1, wherein the administration of the single compound or a pharmaceutically acceptable salt or solvate thereof, is parenteral.

61. The method of claim 1, wherein the administration of the single compound or a pharmaceutically acceptable salt or solvate there is intra-articular.

62. The method of claim 61, wherein the intra-articular administration is ultrasound-guided.

63. The method of claim 61, wherein the administration is directed to one or more joints.

64. The method of claim 63, wherein the one or more joints is selected from the group consisting of: shoulder, elbow, wrist, finger, sacroiliac, hip, knee, ankle, toe, and neck.

65. (canceled)

66. The method of claim 61, wherein the single compound or a pharmaceutically acceptable salt or solvate thereof, is administered once.

67. (canceled)

68. The method of claim 61, wherein the single compound or a pharmaceutically acceptable salt or solvate thereof, is administered more than once with each injection separated by at least 1 week.

69. (canceled)

70. The method of claim 61, wherein the single compound or a pharmaceutically acceptable salt or solvate thereof, is administered more than once with each injection separated by at least 3 months.

71. (canceled)

72. The method of claim 61, wherein the single compound or a pharmaceutically acceptable salt or solvate is administered more than once with each injection separated by 3 months to 60 months.

73. The method of claim 1, wherein the first compound, the second compound, and the dual DYRK1A/CLK2 and/or CLK3 inhibitor are each independently selected from a compound of Formula (I): 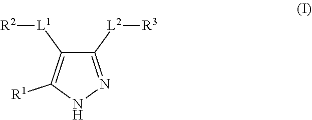
or a pharmaceutically acceptable salt or solvate thereof, wherein:
R¹ is selected from the group consisting of H, halide, and unsubstituted —(C₁₋₃ alkyl);
R² is selected from the group consisting of unsubstituted —(C₁₋₉ alkyl), unsubstituted —(C₂₋₉ alkenyl), unsubstituted —(C₁₋₉ haloalkyl), —(C₁₋₂ alkylene)ₚ(C₃₋₆ carbocyclyl) optionally substituted with 1-12 R⁴, -monocyclic heterocyclyl optionally substituted with 1-10 R⁵, -phenyl optionally substituted with 1-5 R⁶, -heteroaryl optionally substituted with 1-4 R⁷, —CO₂R⁸, —OR⁹, and —(C═O)R¹⁰; wherein —(C₁₋₄ alkylene) is optionally substituted with one or more substituents as defined anywhere herein;
R³ is selected from the group consisting of -heterocyclyl optionally substituted with 1-10 R¹¹, —(C₁₋₄ alkylene)ₚphenyl optionally substituted with 1-5 R¹², -heteroaryl optionally substituted with 1-4 R¹³, and —(C₁₋₄ alkylene)OR¹⁴; wherein each —(C₁₋₄ alkylene) is, independently, optionally substituted with one or more substituents as defined anywhere herein;
each R⁴ is halide;
each R⁵ is independently selected from the group consisting of halide and unsubstituted —(C₁₋₉ alkyl);
each R⁶ is independently selected from the group consisting of unsubstituted —(C₁₋₉ alkyl), unsubstituted —(C₁₋₉ haloalkyl), —OR¹⁵, and —(C₁₋₄ alkylene)ₚN(R¹⁶)₂; wherein —(C₁₋₄ alkylene) is optionally substituted with one or more substituents as defined anywhere herein;
each R⁷ is independently selected from the group consisting of halide, unsubstituted —(C₁₋₉ alkyl), unsubstituted —(C₁₋₉ haloalkyl), —OR¹⁵, —CO₂R¹⁷, —NR¹⁸(C═O)R¹⁹, —(C₁₋₄ alkylene)ₚheterocyclyl optionally substituted with 1-10 R²⁰, and —(C₁₋₄ alkylene)ₚN(R¹⁶)₂; wherein each —(C₁₋₄ alkylene) is, independently, optionally substituted with one or more substituents as defined anywhere herein;
R⁸ is unsubstituted —(C₁₋₉ alkyl);
R⁹ is unsubstituted —(C₁₋₉ alkyl);
R¹⁰ is -aryl optionally substituted with 1-5 R²¹;
each R¹¹ is independently selected from the group consisting of halide and unsubstituted —(C₁₋₉ alkyl);
each R¹² is independently selected from the group consisting of —(C₁₋₄ alkylene)ₚheterocyclyl optionally substituted with 1-10 R²⁰, -aryl optionally substituted with 1-5 R²², —(C₁₋₄ alkylene)N(R¹⁶)₂, and —OR²³; wherein each —(C₁₋₄ alkylene) is, independently, optionally substituted with one or more substituents as defined anywhere herein;
each R¹³ is independently selected from the group consisting of halide, unsubstituted —(C₁₋₉ alkyl), unsubstituted —(C₁₋₉ haloalkyl), —(C₁₋₄ alkylene)ₚN(R¹⁶)₂, —OR²³, —(C₁₋₄ alkylene)ₚheterocyclyl optionally substituted with 1-10 R²⁰, -aryl optionally substituted with 1-5 R²², and -heteroaryl optionally substituted with 1-4 R²⁴; wherein —(C₁₋₄ alkylene) is optionally substituted with one or more substituents as defined anywhere herein;
R¹⁴ is selected from the group consisting of unsubstituted —(C₁₋₄ alkyl) and -aryl optionally substituted with 1-5 R²²;
each R¹⁵ is independently selected from the group consisting of unsubstituted —(C₁₋₉ alkyl) and -heterocyclyl optionally substituted with 1-10 R²⁰;
each R¹⁶ is independently selected from the group consisting of H and unsubstituted —(C₁₋₉ alkyl);
each R¹⁷ is unsubstituted —(C₁₋₉ alkyl);
each R¹⁸ is independently selected from the group consisting of H and unsubstituted —(C₁₋₉ alkyl);
each R¹⁹ is unsubstituted —(C₁₋₉ alkyl);
each R²⁰ is independently selected from the group consisting of halide and unsubstituted —(C₁₋₉ alkyl);
each R²¹ is independently selected from the group consisting of halide and unsubstituted —(C₁₋₉ alkyl);
each R²² is independently selected from the group consisting of halide and unsubstituted —(C₁₋₉ alkyl);
each R²³ is independently selected from the group consisting of unsubstituted —(C₁₋₉ alkyl), —(C₁₋₄ alkylene)OR²⁵, and —(C₁₋₄ alkylene)ₚheterocyclyl optionally substituted with 1-10 R²⁰; wherein each —(C₁₋₄ alkylene) is, independently, optionally substituted with one or more substituents as defined anywhere herein;
each R²⁴ is independently selected from the group consisting of halide and unsubstituted —(C₁₋₉ alkyl);
each R²⁵ is independently selected from the group consisting of H and unsubstituted —(C₉ alkyl);
L¹ is selected from the group consisting of a bond, —CH═CH—, —C≡C—, —(CH₂)ₚNR¹⁸(C═O)—, —(C═O)NR¹⁸(CH₂)ₚ—, —NR¹⁸(C═O)NR¹⁸—, —NH(CH₂)ₚ—, and —(CH₂)ₚNH—;
L² is selected from the group consisting of a bond, —(C═O)NR¹⁸—, —NR¹⁸(C═O)—, —NHCH₂—, and —CH₂NH—; and
each p is independently an integer of 0 or 1.

74. The method of claim 1, wherein the first compound, the second compound, and the dual DYRK1A/CLK2 and/or CLK3 inhibitor are each independently selected from a compound of Formula (II): 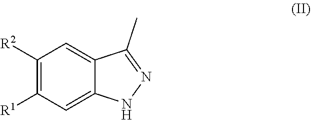
or a pharmaceutically acceptable salt or solvate thereof, wherein:
R¹ is selected from the group consisting of H and halide;
R² is a 6-membered -heteroaryl optionally substituted with 1-4 R³;
each R³ is selected from the group consisting of —OR⁴, —NHR⁵, and —(C₁₋₄ alkylene)ₚheterocyclyl optionally substituted with 1-10 R⁶; wherein —(C₁₋₄ alkylene) is optionally substituted with one or more substituents as defined anywhere herein;
each R⁴ is independently selected from the group consisting of -heterocyclyl optionally substituted with 1-10 R⁷ and —CH₂CH(R⁸)NH₂;
each R⁵ is independently selected from the group consisting of —(C₁₋₄ alkylene)ₚheterocyclyl optionally substituted with 1-10 R⁹ and -carbocyclyl optionally substituted with 1-12 R¹⁰; wherein —(C₁₋₄ alkylene) is optionally substituted with one or more substituents as defined anywhere herein;
each R⁶ is independently selected from the group consisting of halide, —NH₂, —OH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁷ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁸ is independently selected from the group consisting of —(C₁₋₄ alkylene)aryl optionally substituted with 1-5 R¹¹ and —(C₁₋₄ alkylene)heteroaryl optionally substituted with 1-4 R¹²; wherein each —(C₁₋₄ alkylene) is, independently, optionally substituted with one or more substituents as defined anywhere herein;
each R⁹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁰ is independently selected from the group consisting of halide, —OH, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹² is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); and
each p is independently 0 or 1.

75. The method of claim 1, wherein the first compound, the second compound, and the dual DYRK1A/CLK2 and/or CLK3 inhibitor are each independently selected from a compound of Formula (III): 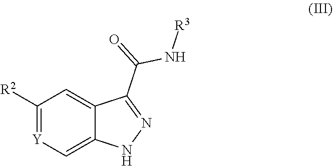
or a pharmaceutically acceptable salt or solvate thereof, wherein:
R¹ is selected from the group consisting of H, halide, and methyl;
R² is a -heteroaryl optionally substituted with 1-4 R⁴;
R³ is selected from the group consisting of H, -aryl optionally substituted with 1-5 R⁵, -heteroaryl optionally substituted with 1-4 R⁶, —C₁₋₆ alkyl optionally substituted with (i) phenyl optionally substituted with 1-5 R¹¹ or (ii) —OR¹⁵, and carbocyclyl optionally substituted with phenyl;
each R⁴ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚN(R⁷)(R⁸), —NHC(═O)R⁹, —(C₁₋₄ alkylene)ₚOR¹⁰, unsubstituted -carbocyclyl, —(C₁₋₄ alkylene)ₚheterocyclyl optionally substituted with 1-10 R¹⁴, —(C₁₋₄ alkylene)ₚaryl optionally substituted with 1-5 R¹¹, and —(C₁₋₄ alkylene)ₚheteroaryl optionally substituted with 1-4 R¹²; wherein each —(C₁₋₄ alkylene) is, independently, optionally substituted with one or more substituents as defined anywhere herein;
each R⁵ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;
each R⁶ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;
each R⁷ is independently selected from the group consisting of H, unsubstituted —(C₁₋₆ alkyl), unsubstituted —(C₂₋₆ alkenyl), and unsubstituted —(C₂₋₆ alkynyl);
each R⁸ is independently selected from the group consisting of H, unsubstituted —(C₁₋₆ alkyl), unsubstituted —(C₂₋₆ alkenyl), unsubstituted —(C₂₋₆ alkynyl), and -heterocyclyl optionally substituted with 1-10 R²¹;
alternatively, R⁷ and R⁸ are taken together to form a -heterocyclyl ring optionally substituted with 1-10 R²¹;
each R⁹ is independently selected from the group consisting of —N(R²²)₂, -carbocyclyl optionally substituted with 1-12 R²³, -heterocyclyl optionally substituted with 1-10 R²¹, and -aryl optionally substituted with 1-5 R²⁴;
each R¹⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R²¹;
each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹² is independently selected from the group consisting of halide, —(C₁₋₄ alkylene)ₚOH, unsubstituted —(C₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;
each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁴ is independently selected from the group consisting of halide, —(C₁₋₄ alkylene)ₚOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;
each R¹⁵ is independently selected from the group consisting of H, unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;
alternatively, two adjacent R¹⁵ are taken together to form a -heterocyclyl ring optionally substituted with 1-10 R²¹;
each R¹⁶ is independently selected from the group consisting of unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;
each R¹⁷ is independently selected from the group consisting of H, unsubstituted —(C₁₋₆ alkyl), unsubstituted —(C₂₋₆ alkenyl), and unsubstituted —(C₂₋₆ alkynyl);
each R¹⁸ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)N(CH₃)₂, and -heterocyclyl ring optionally substituted with 1-10 R²¹; wherein —(C₁₋₄ alkylene) is optionally substituted with one or more substituents as defined anywhere herein;
each R¹⁹ is independently selected from the group consisting of unsubstituted —(C₁₋₆ alkyl), unsubstituted —(C₂₋₆ alkenyl), and unsubstituted —(C₂₋₆ alkynyl).
each R²⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —CH(CH₂OH)₂, —(C₁₋₄ alkylene)ₚheterocyclyl ring optionally substituted with 1-10 R²¹, and -aryl optionally substituted with 1-5 R²⁴; wherein —(C₁₋₄ alkylene) is optionally substituted with one or more substituents as defined anywhere herein;
each R²¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²² is independently selected from the group consisting of unsubstituted —(C₁₋₆ alkyl), unsubstituted —(C₂₋₆ alkenyl), and unsubstituted —(C₂₋₆ alkynyl);
each R²³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆haloalkyl);
each R²⁴ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
Y is selected from the group consisting of —C(R¹)═ and —N═; and
each p is independently 0 or 1.

76. The method of claim 1, wherein the first compound, the second compound, and the dual DYRK1A/CLK2 and/or CLK3 inhibitor are each independently selected from a compound of Formula (IV): 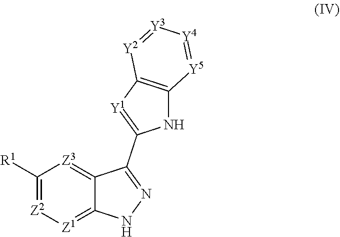
or a pharmaceutically acceptable salt or solvate thereof, wherein:
R¹ is a -heteroaryl optionally substituted with 1-2 R³;
R² is selected from the group consisting of H, halide, -aryl optionally substituted with 1-5 R⁴-heteroaryl optionally substituted with 1-4 R⁵, and -heterocyclyl ring optionally substituted with 1-10 R⁶;
each R³ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R⁷, —C(═O)N(R⁸)₂, —NHC(═O)R⁹, —(C₁₋₄ alkylene)ₚN(R¹⁰)(R¹¹), —(C₁₋₄ alkylene)ₚOR¹², and -carbocyclyl optionally substituted with 1-12 R¹³; wherein each —(C₁₋₄ alkylene) is, independently, optionally substituted with one or more substituents as defined anywhere herein;
each R⁴ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚNHSO₂R¹⁴, —NR¹⁵(C₁₋₄ alkylene)NR¹⁵R¹⁶, —(C₁₋₄ alkylene)ₚNR¹⁵R¹⁶, —OR¹⁷, and -heterocyclyl optionally substituted with 1-10 R¹⁹; wherein each —(C₁₋₄ alkylene) is, independently, optionally substituted with one or more substituents as defined anywhere herein;
each R⁵ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and —C(═O)R¹⁸;
each R⁶ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁷ is independently selected from the group consisting of halide, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁸ is independently selected from the group consisting of H, unsubstituted —(C₁₋₆ alkyl), unsubstituted —(C₂₋₆ alkenyl), unsubstituted —(C₂₋₆ alkynyl), -heterocyclyl optionally substituted with 1-10 R¹⁹, —(C₁₋₄ alkylene)ₚcarbocyclyl optionally substituted with 1-12 R²⁰; wherein —(C₁₋₄ alkylene) is optionally substituted with one or more substituents as defined anywhere herein;
each R⁹ is independently selected from the group consisting of unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²²)₂; wherein each —(C₁₋₄ alkylene) is, independently, optionally substituted with one or more substituents as defined anywhere herein;
each R¹⁰ is independently selected from the group consisting of H, unsubstituted —(C₁₋₆ alkyl), unsubstituted —(C₂₋₆ alkenyl), and unsubstituted —(C₂₋₆ alkynyl);
each R¹¹ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)ₚcarbocyclyl optionally substituted with 1-12 R²⁰; and —(C₁₋₄ alkylene)ₚaryl optionally substituted with 1-5 R²¹; wherein each —(C₁₋₄ alkylene) is, independently, optionally substituted with one or more substituents as defined anywhere herein;
each R¹² is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²²)₂; wherein each —(C₁₋₄ alkylene) is, independently, optionally substituted with one or more substituents as defined anywhere herein;
each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁴ is independently selected from the group consisting of unsubstituted —(C₁₋₆ alkyl), unsubstituted —(C₂₋₆ alkenyl), and unsubstituted —(C₂₋₆ alkynyl);
each R¹⁵ is independently selected from the group consisting of H, unsubstituted —(C₁₋₆ alkyl), unsubstituted —(C₂₋₆ alkenyl), and unsubstituted —(C₂₋₆ alkynyl);
each R¹⁶ is independently selected from the group consisting of H, unsubstituted —(C₁₋₆ alkyl), unsubstituted —(C₂₋₆ alkenyl), and unsubstituted —(C₂₋₆ alkynyl);
each R¹⁷ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, and, —(C₁₋₄ alkylene)ₚN(R²²)₂; wherein each —(C₁₋₄ alkylene) is, independently, optionally substituted with one or more substituents as defined anywhere herein;
each R¹⁸ is independently selected from the group consisting of unsubstituted —(C₁₋₆ alkyl), unsubstituted —(C₂₋₆ alkenyl), and unsubstituted —(C₂₋₆ alkynyl);
each R¹⁹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²⁰ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²² is independently selected from the group consisting of H, unsubstituted —(C₁₋₆ alkyl), unsubstituted —(C₂₋₆ alkenyl), and unsubstituted —(C₂₋₆ alkynyl);
each R²³ is independently selected from the group consisting of H and halide;
R²⁴ is selected from the group consisting of H, halide, and —OR¹⁷;
Y¹ is selected from the group consisting of —CH═ and —N═;
Y² is selected from the group consisting of —C(R²)═ and —N═;
with the proviso that when Y¹ is —N═ then Y² is —C(R²)═;
Y³ is selected from the group consisting of —C(R²⁴)═ and —N═;
Y⁴ and Y⁵ are independently selected from the group consisting of —C(R²³)═ and —N═;
Z¹, Z², and Z³ are independently selected from the group consisting of —C(R²³)═ and —N═;
if Y² is nitrogen then Y³, Y⁴, and Y⁵ are carbon, and R² is absent;
if Y³ is nitrogen then Y⁴ and Y⁵ are carbon;
if Y⁴ is nitrogen then Y³ and Y⁵ are carbon;
if Y⁵ is nitrogen then Y³ and Y⁴ are carbon;
if Z¹ is nitrogen then Z² and Z³ are carbon;
if Z² is nitrogen then Z¹ and Z³ are carbon;
if Z³ is nitrogen then Z¹ and Z² are carbon; and
each p is independently 0 or 1.

77. The method of claim 1, wherein the first compound, the second compound, and the dual DYRK1A/CLK2 and/or CLK3 inhibitor are each independently selected from a compound of Formula (V): 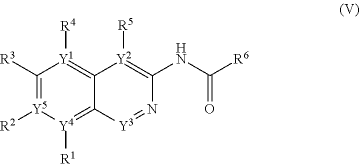
or a pharmaceutically acceptable salt or solvate thereof, wherein:
R¹, R², R⁴, and R⁵ are independently absent or selected from the group consisting of H, halide, unsubstituted —(C₁₋₃ haloalkyl), and unsubstituted —(C₁₋₃ alkyl);
R³ is selected from the group consisting of -aryl optionally substituted with 1-5 R⁷ and -heteroaryl optionally substituted with 1-4 R⁸;
R⁶ is selected from the group consisting of —(C₁₋₄ alkylene)ₚaryl optionally substituted with 1-5 R⁹, —(C₂₋₄ alkenylene)ₚaryl optionally substituted with 1-5 R⁹, —(C₁₋₄ alkylene)ₚheteroaryl optionally substituted with 1-6 R¹⁰; —(C₁₋₄ alkylene)ₚheterocyclyl optionally substituted with 1-10 R¹¹, —(C₁₋₄ alkylene)ₚcarbocyclyl optionally substituted with 1-12 R¹², —(C₁₋₄ alkylene)N(R¹³)(R¹⁴), —N(R¹⁵)(R¹⁶), —CF(C₁₋₉ alkyl)₂, —(C₁₋₄ alkylene)ₚO(C₃₋₉ alkyl), and —(C₂₋₉ alkynyl) optionally substituted with one or more halides; wherein each alkyl of —CF(C₁₋₉ alkyl)₂ is, independently, optionally substituted with one or more halides; wherein each —(C₁₋₄ alkylene) is, independently, optionally substituted with one or more substituents as defined anywhere herein; wherein —(C₁₋₄ alkenylene) is, optionally substituted with one or more substituents as defined anywhere herein;
R⁷ is selected from the group consisting of halide and —N(R¹⁷)₂;
each R⁸ is independently selected from the group consisting of H, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —N(R¹⁵)(R¹⁸), —(C₁₋₄ alkylene)ₚXR¹⁹, —C(═O)N(R¹⁸)₂, —(C₁₋₄ alkylene)ₚheterocyclyl optionally substituted with 1-10 R²⁰, and -carbocyclyl optionally substituted with 1-12 R²¹; wherein each —(C₁₋₄ alkylene) is, independently, optionally substituted with one or more substituents as defined anywhere herein;
alternatively, two adjacent R⁸ are taken together to form a ring which is selected from the group consisting of -heterocyclyl optionally substituted with 1-10 R²² and -carbocyclyl optionally substituted with 1-12 R²¹;
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —XR²³, —C(═O)N(R¹⁸)₂, —(C₁₋₄ alkylene)ₚN(R²⁴)₂, —(C₁₋₄ alkylene)ₚheterocyclyl optionally substituted with 1-10 R²², and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
each R¹⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —XR²³, —C(═O)N(R¹⁸)₂, —(C₁₋₄ alkylene)ₚN(R²⁴)₂, —(C₁₋₄ alkylene)ₚheterocyclyl optionally substituted with 1-10 R²², and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
each R¹¹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚOR¹⁹, —(C₁₋₄ alkylene)ₚheterocyclyl optionally substituted with 1-10 R²², —(C₁₋₄ alkylene)ₚcarbocyclyl optionally substituted with 1-12 R²¹, —N(R¹⁵)(R²⁵), —C(═O)(R²⁶), —(C₁₋₄ alkylene)C(═O)OR²⁷, —(C₁₋₄ alkylene)aryl optionally substituted with one or more halides, —(C₁₋₄ alkylene)ₚheteroaryl optionally substituted with one or more halides, and —SO₂(R²⁸); wherein each —(C₁₋₄ alkylene) is, independently, optionally substituted with one or more substituents as defined anywhere herein;
alternatively, two R¹¹ attached to the same carbon atom can together represent ═O to form a carbonyl group;
each R¹² is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚOR¹⁹, —N(R¹⁵)(R²⁹), —C(═O)(R²⁶), —C(═O)OR²⁷, —(C₁₋₄ alkylene)ₚheterocyclyl optionally substituted with 1-10 R²², and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
R¹³ is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚheterocyclyl optionally substituted with 1-10 R²⁰, and -carbocyclyl optionally substituted with 1-12 R²¹; wherein —(C₁₋₄ alkylene) is, optionally substituted with one or more substituents as defined anywhere herein;
R¹⁴ is selected from the group consisting of unsubstituted —(C₁₋₉ alkyl), unsubstituted —(C₂₋₉ alkenyl), unsubstituted —(C₂₋₉ alkynyl), unsubstituted —(C₁₋₉ haloalkyl), —(C₁₋₄ alkylene)ₚheterocyclyl optionally substituted with 1-10 R²⁰, and -carbocyclyl optionally substituted with 1-12 R²¹; wherein —(C₁₋₄ alkylene) is, optionally substituted with one or more substituents as defined anywhere herein;
each R¹⁵ is selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
R¹⁶ is selected from the group consisting of —(C₁₋₄ alkylene)ₚheterocyclyl optionally substituted with 1-10 R²⁰, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
R¹⁷ is independently selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
alternatively, two adjacent R¹⁷ are taken together to form a -heterocyclyl ring optionally substituted with 1-10 R²²;
R¹⁸ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C═O)R¹⁵, and —(C₁₋₄ alkylene)ₚheterocyclyl optionally substituted with one or more halides or one or more unsubstituted —(C₁₋₅ alkyl); wherein —(C₁₋₄ alkylene) is, independently, optionally substituted with one or more substituents as defined anywhere herein;
each R¹⁹ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
each R²⁰ independently is selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —N(R¹⁵)₂, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
each R²¹ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN;
each R²² is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —N(R¹⁵)₂, —C(═O)R³⁴, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
each R²³ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)N(R¹⁵)₂, —(C₁₋₄ alkylene)ₚaryl optionally substituted with 1-10 R³⁰, —(C₁₋₄ alkylene)ₚheterocyclyl optionally substituted with 1-12 R³¹, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
each R²⁴ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)N(R¹⁵)₂; wherein each —(C₁₋₄ alkylene) is, independently, optionally substituted with one or more substituents as defined anywhere herein;
each R²⁵ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with 1-10 R³², —(C₁₋₄ alkylene)ₚcarbocyclyl optionally substituted with 1-12 R²¹, —(C₁₋₄ alkylene)OR³³; wherein each —(C₁₋₄ alkylene) is, independently, optionally substituted with one or more substituents as defined anywhere herein;
R²⁶ is selected from the group consisting of H, unsubstituted —(C₃₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or unsubstituted —(C₁₋₅ alkyl), —(C₁₋₄ alkylene)ₚheteroaryl optionally substituted with one or more halides or one or more unsubstituted —(C₁₋₅ alkyl), and —(C₁₋₄ alkylene)ₚheterocyclyl optionally substituted with one or more halides or one or more unsubstituted —(C₁₋₅ alkyl); wherein each —(C₁₋₄ alkylene) is, independently, optionally substituted with one or more substituents as defined anywhere herein;
R²⁷ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or one or more unsubstituted —(C₁₋₅ alkyl), —(C₁₋₄ alkylene)ₚheteroaryl optionally substituted with one or more halides or unsubstituted —(C₁₋₅ alkyl), and —(C₁₋₄ alkylene)ₚheterocyclyl optionally substituted with one or more halides or one or more unsubstituted —(C₁₋₅ alkyl); wherein each —(C₁₋₄ alkylene) is, independently, optionally substituted with one or more substituents as defined anywhere herein;
R²⁸ is selected from the group consisting of unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚaryl optionally substituted with one or more halides or one or more unsubstituted —(C₁₋₅ alkyl), —(C₁₋₄ alkylene)ₚheteroaryl optionally substituted with one or more halides or one or more unsubstituted —(C₁₋₅ alkyl), and —(C₁₋₄ alkylene)ₚheterocyclyl optionally substituted with one or more halides or one or more unsubstituted —(C₁₋₅ alkyl); wherein —(C₁₋₄ alkylene) is, optionally substituted with one or more substituents as defined anywhere herein;
each R²⁹ is selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with 1-10 R³², —(C₁₋₄ alkylene)ₚcarbocyclyl optionally substituted with 1-12 R²¹, —(C₁₋₄ alkylene)OR³³, and —C(═O)O(C₁₋₅ alkyl); wherein each —(C₁₋₄ alkylene) is, independently, optionally substituted with one or more substituents as defined anywhere herein;
each R³⁰ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN;
each R³¹ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, —C(═O)R³⁴, —N(R²⁴)₂, and —(C₁₋₄ alkylene)ₚcarbocyclyl optionally substituted with 1-12 R²¹; wherein each —(C₁₋₄ alkylene) is, independently, optionally substituted with one or more substituents as defined anywhere herein;
each R³² is independently selected from the group consisting of halide and unsubstituted —(C₁₋₅ alkyl);
each R³³ is independently selected from the group consisting of H and unsubstituted —(C₁₋₅ alkyl);
each R³⁴ is independently selected from the group consisting of —O(C₁₋₅ alkyl) and a heteroaryl optionally substituted with 1-6 R³⁵;
each R³⁵ is a -heterocyclyl optionally substituted with one or more halides or one or more unsubstituted —(C₁₋₅ alkyl);
each X is selected from the group consisting of O and S;
Y³ is CH or nitrogen;
Y¹, Y², Y⁴, and Y⁵ are independently selected from the group consisting of CH and nitrogen;
wherein
if Y¹ is nitrogen then Y², Y⁴, and Y⁵ are carbon, Y³ is CH, and R⁴ is absent;
if Y² is nitrogen then Y¹, Y⁴, and Y⁵ are carbon, Y³ is CH, and R⁵ is absent;
if Y³ is nitrogen then Y¹, Y², Y⁴, and Y⁵ are carbon;
if Y⁴ is nitrogen then Y¹, Y², and Y⁵ are carbon, Y³ is CH, and R¹ is absent;
if Y⁵ is nitrogen then Y¹, Y², and Y⁴ are carbon, Y³ is CH, and R² is absent; and
each p is independently 0 or 1.

78. The method of claim 1, wherein the first compound, the second compound, and the dual DYRK1A/CLK2 and/or CLK3 inhibitor are each independently selected from a compound of Formula (VI): 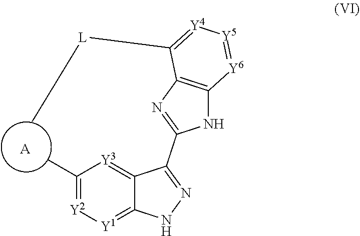
or a pharmaceutically acceptable salt or solvate thereof, wherein:
Ring A is a 5-6-membered heteroaryl optionally substituted with 1-4 R¹;
L is -L¹-L²-L³-L⁴-;
L¹ is selected from the group consisting of unsubstituted —(C₁₋₃ alkylene)-, —NR²—, —NR³(C═O)—, —(C═O)NR³—, and —O—;
L² is selected from the group consisting of unsubstituted —(C₁₋₆ alkylene)- and —NR²—;
L³ is selected from the group consisting of unsubstituted —(C₁₋₆ alkylene)-, —O—, and -carbocyclylene- optionally substituted with one or more halides;
L⁴ is selected from the group consisting of unsubstituted —(C₁₋₆ alkylene)-, —O—, —NR²—, —NR³(C═O)—, —(C═O)NR³—, -arylene- substituted with 1-5 R⁴, and -heteroarylene- optionally substituted with 1-4 R⁵;
with the proviso that —NR²— and —O— are not adjacent to each other;
with the proviso that two —NR²— and/or two —O— are not adjacent to each other;
with the proviso that two —NR³(C═O)— and/or —(C═O)NR³—, are not adjacent to each other;
each R¹ is selected from the group consisting of halide, unsubstituted —(C₁₋₃ alkyl), unsubstituted —(C₁₋₃haloalkyl), and —CN;
each R² is selected from the group consisting of H and unsubstituted —(C₁₋₆ alkyl);
each R³ is selected from the group consisting of H and unsubstituted —(C₁₋₆ alkyl);
each R⁴ is selected from the group consisting of halide, unsubstituted —(C₁₋₆ alkyl), unsubstituted —(C₁₋₆haloalkyl), and —CN;
each R⁵ is selected from the group consisting of halide, unsubstituted —(C₁₋₆ alkyl), unsubstituted —(C₁₋₆haloalkyl), and —CN;
Y¹, Y², Y³, Y⁴, Y⁵, and Y⁶ are independently selected from the group consisting of CH and nitrogen; wherein
if Y¹ is nitrogen then Y² and Y³ are CH;
if Y² is nitrogen then Y¹ and Y³ are CH;
if Y³ is nitrogen then Y¹ and Y² are CH;
if Y⁴ is nitrogen then Y⁵ and Y⁶ are CH;
if Y⁵ is nitrogen then Y⁴ and Y⁶ are CH; and
if Y⁶ is nitrogen then Y⁴ and Y⁵ are CH.

79. The method of claim 1, wherein the first compound, the second compound, and the dual DYRK1A/CLK2 and/or CLK3 inhibitor are each independently selected from a compound of Formula (VII): 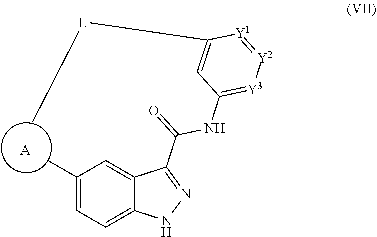
or a pharmaceutically acceptable salt or solvate thereof, wherein:
Ring A is a 5-6-membered heteroaryl optionally substituted with 1-3 R¹;
L is -L¹-L²-L³-L⁴-
L¹ is selected from the group consisting of unsubstituted —(C₁₋₃ alkylene)-, —NR²—, —NR³(C═O)—, —(C═O)NR³—, and —O—;
L² is selected from the group consisting of unsubstituted —(C₁₋₆ alkylene)-, —NR²—, —NR³(C═O)—, and —(C═O)NR³—;
L³ is selected from the group consisting of unsubstituted —(C₁₋₆ alkylene)-, —O—, and carbocyclylene optionally substituted with one or more halides;
L⁴ is selected from the group consisting of unsubstituted —(C₁₋₆ alkylene)-, —O—, —NR²—, —NR³(C═O)—, —(C═O)NR³—, -arylene substituted with 1-5 R⁴, and -heteroarylene optionally substituted with 1-4 R⁵;
with the proviso that —NR²— and —O— are not adjacent to each other;
with the proviso that two —NR²— and/or two —O— are not adjacent to each other;
with the proviso that two —NR³(C═O)— and/or —(C═O)NR³—, are not adjacent to each other;
each R¹ is selected from the group consisting of halide, unsubstituted —(C₁₋₃ alkyl), unsubstituted —(C₁₋₃haloalkyl), and —CN;
each R² is selected from the group consisting of H and unsubstituted —(C₁₋₆ alkyl);
each R³ is selected from the group consisting of H and unsubstituted —(C₁₋₆ alkyl);
each R⁴ is selected from the group consisting of halide, unsubstituted —(C₁₋₆ alkyl), unsubstituted —(C₁₋₆haloalkyl), and —CN;
each R⁵ is selected from the group consisting of halide, unsubstituted —(C₁₋₆ alkyl), unsubstituted —(C₁₋₆haloalkyl), and —CN;
Y¹, Y², and Y³ are independently selected from the group consisting of CH and nitrogen; wherein
if Y¹ is nitrogen then Y² and Y³ are CH;
if Y² is nitrogen then Y¹ and Y³ are CH; and
if Y³ is nitrogen then Y¹ and Y² are CH.

80. The method of claim 1, wherein the first compound, the second compound, and the dual DYRK1A/CLK2 and/or CLK3 inhibitor are each independently selected from a compound of Formula (VIII): 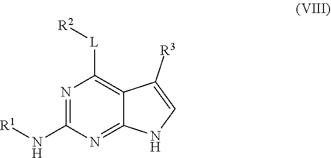
or a pharmaceutically acceptable salt or solvate thereof, wherein:
R¹ is selected from the group consisting of H, unsubstituted —(C₁₋₆ alkyl), unsubstituted —(C₂₋₆ alkenyl), unsubstituted —(C₂₋₆ alkynyl), and -heteroaryl optionally substituted with 1-4 R⁴, -aryl optionally substituted with 1-5 R⁵;
R² is selected from the group consisting of H, —(C₁₋₄ alkylene)ₚheteroaryl optionally substituted with 1-4 R⁶, —(C₁₋₄ alkylene)ₚheterocyclyl optionally substituted with 1-10 R⁷, and —(C₁₋₄ alkylene)ₚcarbocyclyl optionally substituted with 1-12 R⁸; wherein each —(C₁₋₄ alkylene) is, independently, optionally substituted with one or more substituents as defined anywhere herein;
R³ is selected from the group consisting of -heteroaryl optionally substituted with 1-4 R⁹ and -aryl optionally substituted with 1-5 R¹⁰;
each R⁴ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R¹⁴, and —(C₁₋₄ alkylene)ₚcarbocyclyl optionally substituted with 1-12 R¹⁵; wherein each —(C₁₋₄ alkylene) is, independently, optionally substituted with one or more substituents as defined anywhere herein;
each R⁵ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R¹⁴, and —(C₁₋₄ alkylene)ₚcarbocyclyl optionally substituted with 1-12 R¹⁵; wherein each —(C₁₋₄ alkylene) is, independently, optionally substituted with one or more substituents as defined anywhere herein;
each R⁶ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;
each R⁷ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁸ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁹ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;
each R¹⁰ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;
each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹² is independently selected from the group consisting of halide, unsubstituted —(C₁₋₆ alkyl), unsubstituted —(C₂₋₆ alkenyl), and unsubstituted —(C₂₋₆ alkynyl);
each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁴ is independently selected from the group consisting of halide, unsubstituted —(C₁₋₆ alkyl), unsubstituted —(C₂₋₆ alkenyl), and unsubstituted —(C₂₋₆ alkynyl);
each R¹⁵ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
L is selected from the group consisting of a bond, —O—, and —NH—; and
each p is independently 0 or 1.

81.-87. (canceled)

88. The method of claim 1, wherein the dual DYRK1A/CLK2 and/or CLK3 inhibitor is substantially present as a non-stoichiometric hydrate having between 1% and 20% by weight water.

89. The method of claim 1, wherein the single compound is selected from the group consisting of: a compound in Table 3, or a pharmaceutically acceptable salt or solvate thereof, and combinations thereof.

90. The method of claim 1, wherein the dual DYRK1A/CLK2 and/or CLK3 inhibitor is N-(5-(3-(7-(3-fluorophenyl)-3H-imidazo[4,5-c]pyridin-2-yl)-1H-indazol-5-yl)pyridin-3-yl)-3-methylbutanamide, or a pharmaceutically acceptable salt or solvate thereof.

91. The method of claim 90, wherein the N-(5-(3-(7-(3-fluorophenyl)-3H-imidazo[4,5-c]pyridin-2-yl)-1H-indazol-5-yl)pyridin-3-yl)-3-methylbutanamide is substantially present as a non-stoichiometric hydrate of Form 1 having between 1% and 20% by weight water.

92.-97. (canceled)

98. The method of claim 1, wherein the single compound or a pharmaceutically acceptable salt or solvate thereof, has an IC₅₀ between about 100 pM and about 10 μM for DYRK1A.

99. The method of claim 1, wherein the single compound or a pharmaceutically acceptable salt or solvate thereof, has an IC₅₀ between about 100 pM and about 10 μM for CLK2 and/or CLK3.

100. The method of claim 1, wherein the single compound or a pharmaceutically acceptable salt or solvate thereof, has an IC₅₀ between about 1 nM and about 1 μM for DYRK1A.

101. The method of claim 1, wherein the single compound or a pharmaceutically acceptable salt or solvate thereof, has an IC₅₀ between about 1 nM and about 1 μM for CLK2 and/or CLK3.

102. The method of claim 1, wherein the single compound or a pharmaceutically acceptable salt or solvate thereof, has an IC₅₀ between about 1 nM and about 100 nM for DYRK1A

103. The method of claim 1, wherein the single compound or a pharmaceutically acceptable salt or solvate thereof, has an IC₅₀ between about 1 nM and about 100 nM for CLK2 and/or CLK3.

104. The method of claim 9, wherein the level of Wnt pathway activity is the level of β-catenin expression.

105. The method of claim 9, wherein the Wnt pathway activity is detection of a mutation in a Wnt pathway gene comprising at least one of gain-of-function mutation in a β-catenin gene, a loss-of-function mutation in an AXIN gene, a loss-of-function mutation in an AXIN2 gene, a loss-of-function mutation in a APC gene, a loss-of-function mutation in a CTNNβ1 gene, a loss-of-function mutation in a Tsc1 gene, a loss-of-function mutation in a Tsc2 gene, a loss-of-function mutation GSK3β gene, a loss-of-function mutation in a SFRP3 gene, a loss-of-function mutation in a Wnt7b gene, a loss-of-function mutation in a WISP1 gene, a loss-of-function mutation in a DKK1 gene, a loss-of-function mutation in a DOTL1 gene, a loss-of-function mutation in a FZDB gene, a loss-of-function mutation in a LRP5 gene, and a loss-of-function mutation in a LRP6 gene.

106. The method of claim 9, wherein the Wnt pathway activity is detection of an elevated level of expression of one or more Wnt-upregulated genes.

107. The method of claim 106, wherein the one or more Wnt-upregulated genes are selected from the group consisting of: CCND1, CXCL12, LRP5, MMP7, MMP9, LEF1, AXIN2, MYC, TCF7L2, TCF7, LRP6, DVL2, BIRC, ERRB2, MAPK8, PKN1, ABCB1, ADAM10, ALEX1, ASCL2, BAMBI, BCL2L2, BIRC5, BMI1, BMP4, CCND1, CD44, CDKN2A, CDXI, CEBPD, CLDN1, COX2, DNMT1, EDN1, EFNB1, ENC1, EPHB2, EPHB3, FGF18, FGFBP, FRA1, FSCN1, FZD7, FZD8, GAST, HEF1, HES1, ID2, ITF2, JAG1, JUN, L1CAM, LAMC2, LGR5, MENA, MET, MMP14, MYB, MYCBP, NOS2, NOTCH2, NRCAM, PLAU, PLAUR, PPARD, S100A4, S100A6, SGK1, SMC3, SOX9, SP5, SRSF3, SUZ12, TCF1, TIAM1, TIMP-1, TN-C, VEGF, WNT-5a, WNT-5b, WNT11, and YAP.

108. The method of claim 1, wherein the biomarker associated with inflammation is selected from the group consisting of cytokines/chemokines, white blood cell count, modified Glasgow Prognostic Score, neutrophil to lymphocyte ratio, platelet to lymphocyte ratio, T17 lymphocytes, C reactive protein, serum amyloid A, reactive oxygen species, reactive nitrogen species, oxidatively/nitrossatively modified DNA or proteins, 3-Nitrotyrosine, 8-oxodg or 8-OHdG, 8-Iso-PGF2alpha, malonaldehyde, 4-Hydroxynonenal prostaglandin levels, COX2 expression, NF-κB activation, STAT3 activation, cartilage oligomeric matrix protein, ARGS, ADAMTS5, and combinations thereof.

109. The method of claim 108, wherein the biomarker is a cytokine.

110. The method of claim 109, wherein the cytokine is selected from the group consisting of IL-1α, IL-1β, IL-2, IL-4, IL-5, IL-6, IL-7, IL-8, IL-10, IL-12/IL23p40, IL-13, IL-15, IL-16, IL-17A, IL-17F, IL-21, IL-23, TNFα, TNF-β, IFN-γ, CXCL1, CD38, CD40, CD69, IgG, IP-10, L-17A, MCP-1, PGE2, sIL-2, sIL-6, and combinations thereof.

111.-112. (canceled)

113. The method of claim 1, wherein the single compound or a pharmaceutically acceptable salt of solvate thereof, has an EC₅₀ between about 100 pM and about 1 μM in an assay measuring Wnt pathway activity.

114. The method of claim 1, wherein the single compound or a pharmaceutically acceptable salt of solvate thereof, has at least 30-fold selectivity for CLK2 and/or CLK3 compared to a reference CLK2 and/or CLK3 inhibitor and at least 30-fold selectivity for DYRK1A compared to a reference DYRK1A inhibitor.

115. The method of claim 1, wherein the subject has a total WOMAC score of 36 to 72.

116. The method of claim 1, wherein the subject has a Kellgren-Lawrence grade of 2 or 3.

117. The method of claim 1, wherein the subject has a score of X on the Numeric Pain Rating Scale.

118. The method of claim 1, wherein the method increases a joint space width by at least 0.15 mm from the point of intra-articular administration.

119. The method of claim 1, wherein the method results in an increase in the cartilage thickness by at least 0.15 mm from the point of intra-articular administration.

120. The method of claim 119, wherein the subject has about 2 to about 4 mm of baseline cartilage prior to intra-articular administration.

121. (canceled)

122. The method of claim 1, wherein administration of the single compound or pharmaceutically acceptable salt or solvate thereof, decreases a subject's WOMAC total score, as compared to the subject's WOMAC total score prior to administration.

123. (canceled)

124. The method of claim 1, wherein administration of the single compound or pharmaceutically acceptable salt or solvate thereof, decreases a subject's WOMAC function score, as compared to the subject's WOMAC function score prior to administration.

125. (canceled)

126. The method of claim 1, wherein administration of the single compound or pharmaceutically acceptable salt or solvate thereof, decreases a subject's WOMAC pain score, as compared to the subject's WOMAC pain score prior to administration.

127. (canceled)

128. The method of claim 1, wherein administration of the single compound, or pharmaceutically acceptable salt or solvate thereof, decreases a subject's WOMAC stiffness score, as compared to the subject's WOMAC stiffness score prior to administration.

129. The method of claim 1, wherein administration of the single compound, or pharmaceutically acceptable salt or solvate thereof, decreases a subject's NRS score, as compared to the subject's NRS score prior to administration.

130.-131. (canceled)

132. A pharmaceutical composition comprising a therapeutically effective amount of a single compound, wherein the single compound inhibits CLK2 and/or CLK3 at an IC₅₀ value of less than 100 nM and DYRK1A at an IC₅₀ value of less than 100 nM, or a pharmaceutically acceptable salt or solvate thereof, and one or more pharmaceutical excipients

133. (canceled)

134. The pharmaceutical combination of claim 132, further comprising at least one pharmaceutically acceptable carrier.


SEQUENCE LISTING (49 sequences)

SEQ ID NO: 1 - protein, 484 aa, Artificial
  Human CLK1 protein isoform 1
MRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRRCKPLKEFMLSQDVEHERLFDLIQKMLEYDPAKRITLREALKHPFFDLLKKSI

SEQ ID NO: 2 - DNA, 1455 bp, Artificial
  Human CLK1 cDNA isoform 1
atgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatag

SEQ ID NO: 3 - protein, 526 aa, Artificial
  Human CLK1 protein isoform 2
MAAGRRPASALWPERRGSPLRGDLLGFQNVREPSSCGETLSGMRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRRCKPLKEFMLSQDVEHERLFDLIQKMLEYDPAKRITLREALKHPFFDLLKKSI

SEQ ID NO: 4 - DNA, 1581 bp, Artificial
  Human CLK1 cDNA isoform 2
atggcggctgggcggaggccggcttcggccctgtggccggaaaggcgaggctccccgttgaggggggatttgctggggttccagaatgtgcgtgagccaagcagctgtggggaaacgttgtctggaatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatag

SEQ ID NO: 5 - protein, 499 aa, Artificial
  Human CLK2 protein isoform 1
MPHPRRYHSSERGSRGSYREHYRSRKHKRRRSRSWSSSSDRTRRRRREDSYHVRSRSSYDDRSSDRRVYDRRYCGSYRRNDYSRDRGDAYYDTDYRHSYEYQRENSSYRSQRSSRRKHRRRRRRSRTFSRSSSQHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 6 - DNA, 1500 bp, Artificial
  Human CLK2 cDNA isoform 1
atgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcagcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 7 - protein, 498 aa, Artificial
  Human CLK2 protein isoform 2
MPHPRRYHSSERGSRGSYREHYRSRKHKRRRSRSWSSSSDRTRRRRREDSYHVRSRSSYDDRSSDRRVYDRRYCGSYRRNDYSRDRGDAYYDTDYRHSYEYQRENSSYRSQRSSRRKHRRRRRRSRTFSRSSSHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 8 - DNA, 1497 bp, Artificial
  Human CLK2 cDNA isoform 2
atgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 9 - protein, 271 aa, Artificial
  Human CLK2 protein isoform 3
MFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 10 - DNA, 816 bp, Artificial
  Human CLK2 cDNA isoform 3
atgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 11 - protein, 497 aa, Artificial
  Human CLK2 protein isoform 4
MPHPRRYHSSERGSRGSYREHYRSRKHKRRRSRSWSSSSDRTRRRRREDSYHVRSRSYDDRSSDRRVYDRRYCGSYRRNDYSRDRGDAYYDTDYRHSYEYQRENSSYRSQRSSRRKHRRRRRRSRTFSRSSSHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 12 - DNA, 1494 bp, Artificial
  Human CLK2 cDNA isoform 4
atgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 13 - protein, 638 aa, Artificial
  Human CLK3 protein isoform 1
MPVLSARRRELADHAGSGRRSGPSPTARSGPHLSALRAQPARAAHLSGRGTYVRRDTAGGGPGQARPLGPPGTSLLGRGARRSGEGWCPGAFESGARAARPPSRVEPRLATAASREGAGLPRAEVAAGSGRGARSGEWGLAAAGAWETMHHCKRYRSPEPDPYLSYRWKRRRSYSREHEGRLRYPSRREPPPRRSRSRSHDRLPYQRRYRERRDSDTYRCEERSPSFGEDYYGPSRSRHRRRSRERGPYRTRKHAHHCHKRRTRSCSSASSRSQQSSKRSSRSVEDDKEGHLVCRIGDWLQERYEIVGNLGEGTFGKVVECLDHARGKSQVALKIIRNVGKYREAARLEINVLKKIKEKDKENKFLCVLMSDWFNFHGHMCIAFELLGKNTFEFLKENNFQPYPLPHVRHMAYQLCHALRFLHENQLTHTDLKPENILFVNSEFETLYNEHKSCEEKSVKNTSIRVADFGSATFDHEHHTTIVATRHYRPPEVILELGWAQPCDVWSIGCILFEYYRGFTLFQTHENREHLVMMEKILGPIPSHMIHRTRKQKYFYKGGLVWDENSSDGRYVKENCKPLKSYMLQDSLEHVQLFDLMRRMLEFDPAQRITLAEALLHPFFAGLTPEERSFHTSRNPSR

SEQ ID NO: 14 - DNA, 1918 bp, Artificial
  Human CLK3 cDNA isoform 1
aatgcccgtcctctccgcgcgcaggagggagttggcggaccacgcggggtcggggcgacggagcgggcccagccccacggccaggtcggggccccacctctcggctctgagagcccagccggcccgggccgcgcacctgtcaggtcgggggacctacgtgcgccgcgacacggcgggaggcgggccgggccaggctcgtcccctcggccctcccggaactagtctcctaggccgcggcgcccgccggagcggagagggctggtgccccggagccttcgagtcgggggctagagcggccaggcctccgagccgggtcgagccgaggctggcgacggctgcgtcacgcgagggggcggggctgccacgggcggaggtcgcagccggaagcggaagaggcgctcggagcggggagtggggcctagctgcagccggagcctgggagacgatgcatcactgtaagcgataccgctcccctgaaccagacccgtacctgagctaccgatggaagaggaggaggtcctacagtcgggaacatgaagggagactgcgatacccgtcccgaagggagcctcccccacgaagatctcggtccagaagccatgaccgcctgccctaccagaggaggtaccgggagcgccgtgacagcgatacataccggtgtgaagagcggagcccatcctttggagaggactactatggaccttcacgttctcgtcatcgtcggcgatcgcgggagagggggccataccggacccgcaagcatgcccaccactgccacaaacgccgcaccaggtcttgtagcagcgcctcctcgagaagccaacagagcagtaagcgcagcagccggagtgtggaagatgacaaggagggtcacctggtgtgccggatcggcgattggctccaagagcgatatgagattgtggggaacctgggtgaaggcacctttggcaaggtggtggagtgcttggaccatgccagagggaagtctcaggttgccctgaagatcatccgcaacgtgggcaagtaccgggaggctgcccggctagaaatcaacgtgctcaaaaaaatcaaggagaaggacaaagaaaacaagttcctgtgtgtcttgatgtctgactggttcaacttccacggtcacatgtgcatcgcctttgagctcctgggcaagaacacctttgagttcctgaaggagaataacttccagccttaccccctaccacatgtccggcacatggcctaccagctctgccacgcccttagatttctgcatgagaatcagctgacccatacagacttgaaaccagagaacatcctgtttgtgaattctgagtttgaaaccctctacaatgagcacaagagctgtgaggagaagtcagtgaagaacaccagcatccgagtggctgactttggcagtgccacatttgaccatgagcaccacaccaccattgtggccacccgtcactatcgcccgcctgaggtgatccttgagctgggctgggcacagccctgtgacgtctggagcattggctgcattctctttgagtactaccggggcttcacactcttccagacccacgaaaaccgagagcacctggtgatgatggagaagatcctagggcccatcccatcacacatgatccaccgtaccaggaagcagaaatatttctacaaagggggcctagtttgggatgagaacagctctgacggccggtatgtgaaggagaactgcaaacctctgaagagttacatgctccaagactccctggagcacgtgcagctgtttgacctgatgaggaggatgttagaatttgaccctgcccagcgcatcacactggccgaggccctgctgcaccccttctttgctggcctgacccctgaggagcggtccttccacaccagccgcaacccaagcagatga

SEQ ID NO: 15 - protein, 490 aa, Artificial
  Human CLK3 protein isoform 2
MHHCKRYRSPEPDPYLSYRWKRRRSYSREHEGRLRYPSRREPPPRRSRSRSHDRLPYQRRYRERRDSDTYRCEERSPSFGEDYYGPSRSRHRRRSRERGPYRTRKHAHHCHKRRTRSCSSASSRSQQSSKRSSRSVEDDKEGHLVCRIGDWLQERYEIVGNLGEGTFGKVVECLDHARGKSQVALKIIRNVGKYREAARLEINVLKKIKEKDKENKFLCVLMSDWFNFHGHMCIAFELLGKNTFEFLKENNFQPYPLPHVRHMAYQLCHALRFLHENQLTHTDLKPENILFVNSEFETLYNEHKSCEEKSVKNTSIRVADFGSATFDHEHHTTIVATRHYRPPEVILELGWAQPCDVWSIGCILFEYYRGFTLFQTHENREHLVMMEKILGPIPSHMIHRTRKQKYFYKGGLVWDENSSDGRYVKENCKPLKSYMLQDSLEHVQLFDLMRRMLEFDPAQRITLAEALLHPFFAGLTPEERSFHTSRNPSR

SEQ ID NO: 16 - DNA, 1473 bp, Artificial
  Human CLK3 cDNA isoform 2
atgcatcactgtaagcgataccgctcccctgaaccagacccgtacctgagctaccgatggaagaggaggaggtcctacagtcgggaacatgaagggagactgcgatacccgtcccgaagggagcctcccccacgaagatctcggtccagaagccatgaccgcctgccctaccagaggaggtaccgggagcgccgtgacagcgatacataccggtgtgaagagcggagcccatcctttggagaggactactatggaccttcacgttctcgtcatcgtcggcgatcgcgggagagggggccataccggacccgcaagcatgcccaccactgccacaaacgccgcaccaggtcttgtagcagcgcctcctcgagaagccaacagagcagtaagcgcagcagccggagtgtggaagatgacaaggagggtcacctggtgtgccggatcggcgattggctccaagagcgatatgagattgtggggaacctgggtgaaggcacctttggcaaggtggtggagtgcttggaccatgccagagggaagtctcaggttgccctgaagatcatccgcaacgtgggcaagtaccgggaggctgcccggctagaaatcaacgtgctcaaaaaaatcaaggagaaggacaaagaaaacaagttcctgtgtgtcttgatgtctgactggttcaacttccacggtcacatgtgcatcgcctttgagctcctgggcaagaacacctttgagttcctgaaggagaataacttccagccttaccccctaccacatgtccggcacatggcctaccagctctgccacgcccttagatttctgcatgagaatcagctgacccatacagacttgaaaccagagaacatcctgtttgtgaattctgagtttgaaaccctctacaatgagcacaagagctgtgaggagaagtcagtgaagaacaccagcatccgagtggctgactttggcagtgccacatttgaccatgagcaccacaccaccattgtggccacccgtcactatcgcccgcctgaggtgatccttgagctgggctgggcacagccctgtgacgtctggagcattggctgcattctctttgagtactaccggggcttcacactcttccagacccacgaaaaccgagagcacctggtgatgatggagaagatcctagggcccatcccatcacacatgatccaccgtaccaggaagcagaaatatttctacaaagggggcctagtttgggatgagaacagctctgacggccggtatgtgaaggagaactgcaaacctctgaagagttacatgctccaagactccctggagcacgtgcagctgtttgacctgatgaggaggatgttagaatttgaccctgcccagcgcatcacactggccgaggccctgctgcaccccttctttgctggcctgacccctgaggagcggtccttccacaccagccgcaacccaagcagatga

SEQ ID NO: 17 - protein, 481 aa, Artificial
  Human CLK4 protein
MRHSKRTHCPDWDSRESWGHESYRGSHKRKRRSHSSTQENRHCKPHHQFKESDCHYLEARSLNERDYRDRRYVDEYRNDYCEGYVPRHYHRDIESGYRIHCSKSSVRSRRSSPKRKRNRHCSSHQSRSKSHRRKRSRSIEDDEEGHLICQSGDVLRARYEIVDTLGEGAFGKVVECIDHGMDGMHVAVKIVKNVGRYREAARSEIQVLEHLNSTDPNSVFRCVQMLEWFDHHGHVCIVFELLGLSTYDFIKENSFLPFQIDHIRQMAYQICQSINFLHHNKLTHTDLKPENILFVKSDYVVKYNSKMKRDERTLKNTDIKVVDFGSATYDDEHHSTLVSTRHYRAPEVILALGWSQPCDVWSIGCILIEYYLGFTVFQTHDSKEHLAMMERILGPIPQHMIQKTRKRKYFHHNQLDWDEHSSAGRYVRRRCKPLKEFMLCHDEEHEKLFDLVRRMLEYDPTQRITLDEALQHPFFDLLKKK

SEQ ID NO: 18 - DNA, 1446 bp, Artificial
  Human CLK4
atgcggcattccaaaagaactcactgtcctgattgggatagcagagaaagctggggacatgaaagctatcgtggaagtcacaagcggaagaggagatctcatagtagcacacaagagaacaggcattgtaaaccacatcaccagtttaaagaatctgattgtcattatttagaagcaaggtccttgaatgagcgagattatcgggaccggagatacgttgacgaatacaggaatgactactgtgaaggatatgttcctagacattatcacagagacattgaaagcgggtatcgaatccactgcagtaaatcttcagtccgcagcaggagaagcagtcctaaaaggaagcgcaatagacactgttcaagtcatcagtcacgttcgaagagccaccgaaggaaaagatccaggagtatagaggatgatgaggagggtcacctgatctgtcaaagtggagacgttctaagagcaagatatgaaatcgtggacactttgggtgaaggagcctttggcaaagttgtagagtgcattgatcatggcatggatggcatgcatgtagcagtgaaaatcgtaaaaaatgtaggccgttaccgtgaagcagctcgttcagaaatccaagtattagagcacttaaatagtactgatcccaatagtgtcttccgatgtgtccagatgctagaatggtttgatcatcatggtcatgtttgtattgtgtttgaactactgggacttagtacttacgatttcattaaagaaaacagctttctgccatttcaaattgaccacatcaggcagatggcgtatcagatctgccagtcaataaattttttacatcataataaattaacccatacagatctgaagcctgaaaatattttgtttgtgaagtctgactatgtagtcaaatataattctaaaatgaaacgtgatgaacgcacactgaaaaacacagatatcaaagttgttgactttggaagtgcaacgtatgatgatgaacatcacagtactttggtgtctacccggcactacagagctcccgaggtcattttggctttaggttggtctcagccttgtgatgtttggagcataggttgcattcttattgaatattaccttggtttcacagtctttcagactcatgatagtaaagagcacctggcaatgatggaacgaatattaggacccataccacaacacatgattcagaaaacaagaaaacgcaagtattttcaccataaccagctagattgggatgaacacagttctgctggtagatatgttaggagacgctgcaaaccgttgaaggaatttatgctttgtcatgatgaagaacatgagaaactgtttgacctggttcgaagaatgttagaatatgatccaactcaaagaattaccttggatgaagcattgcagcatcctttctttgacttattaaaaaagaaatga

SEQ ID NO: 19 - protein, 763 aa, Artificial
  Human DYRK1A protein long isoform
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFTAAVQAMDCETHSPQVRQQFPAPLGWSGTEAPTQVTVETHPVQETTFHVAPQQNALHHHHGNSSHHHHHHHHHHHHHGQQALGNRTRPRVYNSPTNSSSTQDSMEVGHSHHSMTSLSSSTTSSSTSSSSTGNQGNQAYQNRPVAANTLDFGQNGAMDVNLTVYSNPRQETGIAGHPTYQFSANTGPAHYMTEGHLTMRQGADREESPMTGVCVQQSPVASS

SEQ ID NO: 20 - DNA, 2292 bp, Artificial
  Human DYRK1A cDNA long isoform
atgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccaggtgatgcctgatattgtcatgttacagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtggctcatcggggacaagcaacagtgggagagcccggtcggatccgacgcaccagcatcggcacagtggtgggcacttcacagctgccgtgcaggccatggactgcgagacacacagtccccaggtgcgtcagcaatttcctgctcctcttggttggtcaggcactgaagctcctacacaggtcactgttgaaactcatcctgttcaagaaacaacctttcatgtagcccctcaacagaatgcattgcatcatcaccatggtaacagttcccatcaccatcaccaccaccaccaccatcaccaccaccatggacaacaagccttgggtaaccggaccaggccaagggtctacaattctccaacgaatagctcctctacccaagattctatggaggttggccacagtcaccactccatgacatccctgtcttcctcaacgacttcttcctcgacatcttcctcctctactggtaaccaaggcaatcaggcctaccagaatcgcccagtggctgctaataccttggactttggacagaatggagctatggacgttaatttgaccgtctactccaatccccgccaagagactggcatagctggacatccaacataccaattttctgctaatacaggtcctgcacattacatgactgaaggacatctgacaatgaggcaaggggctgatagagaagagtcccccatgacaggagtttgtgtgcaacagagtcctgtagctagctcgtga

SEQ ID NO: 21 - protein, 754 aa, Artificial
  Human DYRK1A protein isoform 1
MHTGGETSACKPSSVRLAPSFSFHAAGLQMAGQMPHSHQYSDRRQPNISDQQVSALSYSDQIQQPLTNQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFTAAVQAMDCETHSPQVRQQFPAPLGWSGTEAPTQVTVETHPVQETTFHVAPQQNALHHHHGNSSHHHHHHHHHHHHHGQQALGNRTRPRVYNSPTNSSSTQDSMEVGHSHHSMTSLSSSTTSSSTSSSSTGNQGNQAYQNRPVAANTLDFGQNGAMDVNLTVYSNPRQETGIAGHPTYQFSANTGPAHYMTEGHLTMRQGADREESPMTGVCVQQSPVASS

SEQ ID NO: 22 - DNA, 2265 bp, Artificial
  Human DYRK1A cDNA isoform 1
atgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtggctcatcggggacaagcaacagtgggagagcccggtcggatccgacgcaccagcatcggcacagtggtgggcacttcacagctgccgtgcaggccatggactgcgagacacacagtccccaggtgcgtcagcaatttcctgctcctcttggttggtcaggcactgaagctcctacacaggtcactgttgaaactcatcctgttcaagaaacaacctttcatgtagcccctcaacagaatgcattgcatcatcaccatggtaacagttcccatcaccatcaccaccaccaccaccatcaccaccaccatggacaacaagccttgggtaaccggaccaggccaagggtctacaattctccaacgaatagctcctctacccaagattctatggaggttggccacagtcaccactccatgacatccctgtcttcctcaacgacttcttcctcgacatcttcctcctctactggtaaccaaggcaatcaggcctaccagaatcgcccagtggctgctaataccttggactttggacagaatggagctatggacgttaatttgaccgtctactccaatccccgccaagagactggcatagctggacatccaacataccaattttctgctaatacaggtcctgcacattacatgactgaaggacatctgacaatgaggcaaggggctgatagagaagagtcccccatgacaggagtttgtgtgcaacagagtcctgtagctagctcgtga

SEQ ID NO: 23 - protein, 529 aa, Artificial
  Human DYRK1A protein isoform 2
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGASAISCSSWLVRH

SEQ ID NO: 24 - DNA, 1590 bp, Artificial
  Human DYRK1A cDNA isoform 2
atgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccaggtgatgcctgatattgtcatgttacagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtgcgtcagcaatttcctgctcctcttggttggtcaggcactga

SEQ ID NO: 25 - protein, 525 aa, Artificial
  Human DYRK1A protein isoform 3
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGAALDARCL

SEQ ID NO: 26 - DNA, 1578 bp, Artificial
  Human DYRK1A cDNA isoform 3
atgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccaggtgatgcctgatattgtcatgttacagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtggagcagcactggatgccaggtgcctttag

SEQ ID NO: 27 - protein, 584 aa, Artificial
  Human DYRK1A protein isoform 4
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFTAAVQAMDCETHSPQVSSHVVHLLVSPAILRWSSTGCQVPLE

SEQ ID NO: 28 - DNA, 1755 bp, Artificial
  Human DYRK1A cDNA isoform 4
atgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccaggtgatgcctgatattgtcatgttacagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtggctcatcggggacaagcaacagtgggagagcccggtcggatccgacgcaccagcatcggcacagtggtgggcacttcacagctgccgtgcaggccatggactgcgagacacacagtccccaggtgagctcgcacgtggttcatttgcttgtgtcacctgccattctcaggtggagcagcactggatgccaggtgcctttagaatga

SEQ ID NO: 29 - protein, 629 aa, Artificial
  Human DYRK1B protein isoform 1
MAVPPGHGPFSGFPGPQEHTQVLPDVRLLPRRLPLAFRDATSAPLRKLSVDLIKTYKHINEVYYAKKKRRAQQAPPQDSSNKKEKKVLNHGYDDDNHDYIVRSGERWLERYEIDSLIGKGSFGQVVKAYDHQTQELVAIKIIKNKKAFLNQAQIELRLLELMNQHDTEMKYYIVHLKRHFMFRNHLCLVFELLSYNLYDLLRNTHFRGVSLNLTRKLAQQLCTALLFLATPELSIIHCDLKPENILLCNPKRSAIKIVDFGSSCQLGQRIYQYIQSRFYRSPEVLLGTPYDLAIDMWSLGCILVEMHTGEPLFSGSNEVDQMNRIVEVLGIPPAAMLDQAPKARKYFERLPGGGWTLRRTKELRKDYQGPGTRRLQEVLGVQTGGPGGRRAGEPGHSPADYLRFQDLVLRMLEYEPAARISPLGALQHGFFRRTADEATNTGPAGSSASTSPAPLDTCPSSSTASSISSSGGSSGSSSDNRTYRYSNRYCGGPGPPITDCEMNSPQVPPSQPLRPWAGGDVPHKTHQAPASASSLPGTGAQLPPQPRYLGRPPSPTSPPPPELMDVSLVGGPADCSPPHPAPAPQHPAASALRTRMTGGRPPLPPPDDPATLGPHLGLRGVPQSTAASS

SEQ ID NO: 30 - DNA, 1890 bp, Artificial
  Human DYRK1B cDNA isoform 1
atggccgtcccaccgggccatggtcccttctctggcttcccagggccccaggagcacacgcaggtattgcctgatgtgcggctactgcctcggaggctgcccctggccttccgggatgcaacctcagccccgctgcgtaagctctctgtggacctcatcaagacctacaagcacatcaatgaggtatactatgcgaagaagaagcggcgggcccagcaggcgccaccccaggattcgagcaacaagaaggagaagaaggtcctgaaccatggttatgatgacgacaaccatgactacatcgtgcgcagtggcgagcgctggctggagcgctacgaaattgactcgctcattggcaaaggctcctttggccaggtggtgaaagcctatgatcatcagacccaggagcttgtggccatcaagatcatcaagaacaaaaaggctttcctgaaccaggcccagattgagctgcggctgctggagctgatgaaccagcatgacacggagatgaagtactatatagtacacctgaagcggcacttcatgttccggaaccacctgtgcctggtatttgagctgctgtcctacaacctgtacgacctcctgcgcaacacccacttccgcggcgtctcgctgaacctgacccggaagctggcgcagcagctctgcacggcactgctctttctggccacgcctgagctcagcatcattcactgcgacctcaagcccgaaaacatcttgctgtgcaaccccaagcgcagcgccatcaagattgtggacttcggcagctcctgccagcttggccagaggatctaccagtatatccagagccgcttctaccgctcacctgaggtgctcctgggcacaccctacgacctggccattgacatgtggtccctgggctgcatccttgtggagatgcacaccggagagcccctcttcagtggctccaatgaggtcgaccagatgaaccgcattgtggaggtgctgggcatcccaccggccgccatgctggaccaggcgcccaaggctcgcaagtactttgaacggctgcctgggggtggctggaccctacgaaggacgaaagaactcaggaaggattaccagggccccgggacacggcggctgcaggaggtgctgggcgtgcagacgggcgggcccgggggccggcgggcgggggagccgggccacagccccgccgactacctccgcttccaggacctggtgctgcgcatgctggagtatgagcccgccgcccgcatcagccccctgggggctctgcagcacggcttcttccgccgcacggccgacgaggccaccaacacgggcccggcaggcagcagtgcctccacctcgcccgcgcccctcgacacctgcccctcttccagcaccgccagctccatctccagttctggaggctccagtggctcctccagtgacaaccggacctaccgctacagcaaccgatattgtgggggccctgggccccctatcacagactgtgagatgaacagcccccaggtcccaccctcccagccgctgcggccctgggcagggggtgatgtgccccacaagacacatcaagcccctgcctctgcctcgtcactgcctgggaccggggcccagttacccccccagccccgataccttggtcgtcccccatcaccaacctcaccaccacccccggagctgatggatgtgagcctggtgggcggccctgctgactgctccccacctcacccagcgcctgccccccagcacccggctgcctcagccctccggactcggatgactggaggtcgtccacccctcccgcctcctgatgaccctgccactctggggcctcacctgggcctccgtggtgtaccccagagcacagcagccagctcgtga

SEQ ID NO: 31 - protein, 589 aa, Artificial
  Human DYRK1B protein isoform 2
MAVPPGHGPFSGFPGPQEHTQVLPDVRLLPRRLPLAFRDATSAPLRKLSVDLIKTYKHINEVYYAKKKRRAQQAPPQDSSNKKEKKVLNHGYDDDNHDYIVRSGERWLERYEIDSLIGKGSFGQVVKAYDHQTQELVAIKIIKNKKAFLNQAQIELRLLELMNQHDTEMKYYIVHLKRHFMFRNHLCLVFELLSYNLYDLLRNTHFRGVSLNLTRKLAQQLCTALLFLATPELSIIHCDLKPENILLCNPKRSAIKIVDFGSSCQLGQRIYQYIQSRFYRSPEVLLGTPYDLAIDMWSLGCILVEMHTGEPLFSGSNEVDQMNRIVEVLGIPPAAMLDQAPKARKYFERLPGGGWTLRRTKELRKDLVLRMLEYEPAARISPLGALQHGFFRRTADEATNTGPAGSSASTSPAPLDTCPSSSTASSISSSGGSSGSSSDNRTYRYSNRYCGGPGPPITDCEMNSPQVPPSQPLRPWAGGDVPHKTHQAPASASSLPGTGAQLPPQPRYLGRPPSPTSPPPPELMDVSLVGGPADCSPPHPAPAPQHPAASALRTRMTGGRPPLPPPDDPATLGPHLGLRGVPQSTAASS

SEQ ID NO: 32 - DNA, 1770 bp, Artificial
  Human DYRK1B cDNA isoform 2
atggccgtcccaccgggccatggtcccttctctggcttcccagggccccaggagcacacgcaggtattgcctgatgtgcggctactgcctcggaggctgcccctggccttccgggatgcaacctcagccccgctgcgtaagctctctgtggacctcatcaagacctacaagcacatcaatgaggtatactatgcgaagaagaagcggcgggcccagcaggcgccaccccaggattcgagcaacaagaaggagaagaaggtcctgaaccatggttatgatgacgacaaccatgactacatcgtgcgcagtggcgagcgctggctggagcgctacgaaattgactcgctcattggcaaaggctcctttggccaggtggtgaaagcctatgatcatcagacccaggagcttgtggccatcaagatcatcaagaacaaaaaggctttcctgaaccaggcccagattgagctgcggctgctggagctgatgaaccagcatgacacggagatgaagtactatatagtacacctgaagcggcacttcatgttccggaaccacctgtgcctggtatttgagctgctgtcctacaacctgtacgacctcctgcgcaacacccacttccgcggcgtctcgctgaacctgacccggaagctggcgcagcagctctgcacggcactgctctttctggccacgcctgagctcagcatcattcactgcgacctcaagcccgaaaacatcttgctgtgcaaccccaagcgcagcgccatcaagattgtggacttcggcagctcctgccagcttggccagaggatctaccagtatatccagagccgcttctaccgctcacctgaggtgctcctgggcacaccctacgacctggccattgacatgtggtccctgggctgcatccttgtggagatgcacaccggagagcccctcttcagtggctccaatgaggtcgaccagatgaaccgcattgtggaggtgctgggcatcccaccggccgccatgctggaccaggcgcccaaggctcgcaagtactttgaacggctgcctgggggtggctggaccctacgaaggacgaaagaactcaggaaggacctggtgctgcgcatgctggagtatgagcccgccgcccgcatcagccccctgggggctctgcagcacggcttcttccgccgcacggccgacgaggccaccaacacgggcccggcaggcagcagtgcctccacctcgcccgcgcccctcgacacctgcccctcttccagcaccgccagctccatctccagttctggaggctccagtggctcctccagtgacaaccggacctaccgctacagcaaccgatattgtgggggccctgggccccctatcacagactgtgagatgaacagcccccaggtcccaccctcccagccgctgcggccctgggcagggggtgatgtgccccacaagacacatcaagcccctgcctctgcctcgtcactgcctgggaccggggcccagttacccccccagccccgataccttggtcgtcccccatcaccaacctcaccaccacccccggagctgatggatgtgagcctggtgggcggccctgctgactgctccccacctcacccagcgcctgccccccagcacccggctgcctcagccctccggactcggatgactggaggtcgtccacccctcccgcctcctgatgaccctgccactctggggcctcacctgggcctccgtggtgtaccccagagcacagcagccagctcgtga

SEQ ID NO: 33 - protein, 601 aa, Artificial
  Human DYRK1B protein isoform 3
MAVPPGHGPFSGFPGPQEHTQVLPDVRLLPRRLPLAFRDATSAPLRKLSVDLIKTYKHINEVYYAKKKRRAQQAPPQDSSNKKEKKVLNHGYDDDNHDYIVRSGERWLERYEIDSLIGKGSFGQVVKAYDHQTQELVAIKIIKNKKAFLNQAQIELRLLELMNQHDTEMKYYIVHLKRHFMFRNHLCLVFELLSYNLYDLLRNTHFRGVSLNLTRKLAQQLCTALLFLATPELSIIHCDLKPENILLCNPKRSAIKIVDFGSSCQLGQRIYQYIQSRFYRSPEVLLGTPYDLAIDMWSLGCILVEMHTGEPLFSGSNEVDQMNRIVEVLGIPPAAMLDQAPKARKYFERLPGGGWTLRRTKELRKDYQGPGTRRLQEDLVLRMLEYEPAARISPLGALQHGFFRRTADEATNTGPAGSSASTSPAPLDTCPSSSTASSISSSGGSSGSSSDNRTYRYSNRYCGGPGPPITDCEMNSPQVPPSQPLRPWAGGDVPHKTHQAPASASSLPGTGAQLPPQPRYLGRPPSPTSPPPPELMDVSLVGGPADCSPPHPAPAPQHPAASALRTRMTGGRPPLPPPDDPATLGPHLGLRGVPQSTAASS

SEQ ID NO: 34 - DNA, 1806 bp, Artificial
  Human DYRK1B cDNA isoform 3
atggccgtcccaccgggccatggtcccttctctggcttcccagggccccaggagcacacgcaggtattgcctgatgtgcggctactgcctcggaggctgcccctggccttccgggatgcaacctcagccccgctgcgtaagctctctgtggacctcatcaagacctacaagcacatcaatgaggtatactatgcgaagaagaagcggcgggcccagcaggcgccaccccaggattcgagcaacaagaaggagaagaaggtcctgaaccatggttatgatgacgacaaccatgactacatcgtgcgcagtggcgagcgctggctggagcgctacgaaattgactcgctcattggcaaaggctcctttggccaggtggtgaaagcctatgatcatcagacccaggagcttgtggccatcaagatcatcaagaacaaaaaggctttcctgaaccaggcccagattgagctgcggctgctggagctgatgaaccagcatgacacggagatgaagtactatatagtacacctgaagcggcacttcatgttccggaaccacctgtgcctggtatttgagctgctgtcctacaacctgtacgacctcctgcgcaacacccacttccgcggcgtctcgctgaacctgacccggaagctggcgcagcagctctgcacggcactgctctttctggccacgcctgagctcagcatcattcactgcgacctcaagcccgaaaacatcttgctgtgcaaccccaagcgcagcgccatcaagattgtggacttcggcagctcctgccagcttggccagaggatctaccagtatatccagagccgcttctaccgctcacctgaggtgctcctgggcacaccctacgacctggccattgacatgtggtccctgggctgcatccttgtggagatgcacaccggagagcccctcttcagtggctccaatgaggtcgaccagatgaaccgcattgtggaggtgctgggcatcccaccggccgccatgctggaccaggcgcccaaggctcgcaagtactttgaacggctgcctgggggtggctggaccctacgaaggacgaaagaactcaggaaggattaccagggccccgggacacggcggctgcaggaggacctggtgctgcgcatgctggagtatgagcccgccgcccgcatcagccccctgggggctctgcagcacggcttcttccgccgcacggccgacgaggccaccaacacgggcccggcaggcagcagtgcctccacctcgcccgcgcccctcgacacctgcccctcttccagcaccgccagctccatctccagttctggaggctccagtggctcctccagtgacaaccggacctaccgctacagcaaccgatattgtgggggccctgggccccctatcacagactgtgagatgaacagcccccaggtcccaccctcccagccgctgcggccctgggcagggggtgatgtgccccacaagacacatcaagcccctgcctctgcctcgtcactgcctgggaccggggcccagttacccccccagccccgataccttggtcgtcccccatcaccaacctcaccaccacccccggagctgatggatgtgagcctggtgggcggccctgctgactgctccccacctcacccagcgcctgccccccagcacccggctgcctcagccctccggactcggatgactggaggtcgtccacccctcccgcctcctgatgaccctgccactctggggcctcacctgggcctccgtggtgtaccccagagcacagcagccagctcgtga

SEQ ID NO: 35 - protein, 601 aa, Artificial
  Human DYRK2 protein isoform 1
MLTRKPSAAAPAAYPTGRGGDSAVRQLQASPGLGAGATRSGVGTGPPSPIALPPLRASNAAAAAHTIGGSKHTMNDHLHVGSHAHGQIQVQQLFEDNSNKRTVLTTQPNGLTTVGKTGLPVVPERQLDSIHRRQGSSTSLKSMEGMGKVKATPMTPEQAMKQYMQKLTAFEHHEIFSYPEIYFLGLNAKKRQGMTGGPNNGGYDDDQGSYVQVPHDHVAYRYEVLKVIGKGSFGQVVKAYDHKVHQHVALKMVRNEKRFHRQAAEEIRILEHLRKQDKDNTMNVIHMLENFTFRNHICMTFELLSMNLYELIKKNKFQGFSLPLVRKFAHSILQCLDALHKNRIIHCDLKPENILLKQQGRSGIKVIDFGSSCYEHQRVYTYIQSRFYRAPEVILGARYGMPIDMWSLGCILAELLTGYPLLPGEDEGDQLACMIELLGMPSQKLLDASKRAKNFVSSKGYPRYCTVTTLSDGSVVLNGGRSRRGKLRGPPESREWGNALKGCDDPLFLDFLKQCLEWDPAVRMTPGQALRHPWLRRRLPKPPTGEKTSVKRITESTGAITSISKLPPPSSSASKLRTNLAQMTDANGNIQQRTVLPKLVS

SEQ ID NO: 36 - DNA, 1806 bp, Artificial
  Human DYRK2 cDNA isoform
atgttaaccaggaaaccttcggccgccgctcccgccgcctacccgaccggccgaggtggggacagcgccgttcgtcagcttcaggcttccccggggctcggtgcaggggccacccggagcggagtggggactggcccgccctcccccatcgccctgccgcctctccgggccagcaacgctgccgccgcagcccacacgattggcggcagtaagcacacaatgaatgatcacctgcatgtcggcagccacgctcacggacagatccaggttcaacagttgtttgaggataacagtaacaagcggacagtgctcacgacacaaccaaatgggcttacaacagtgggcaaaacgggcttgccagtggtgccagagcggcagctggacagcattcatagacggcaggggagctccacctctctaaagtccatggaaggcatggggaaggtgaaagccacccccatgacacctgaacaagcaatgaagcaatacatgcaaaaactcacagccttcgaacaccatgagattttcagctaccctgaaatatatttcttgggtctaaatgctaagaagcgccagggcatgacaggtgggcccaacaatggtggctatgatgatgaccagggatcatatgtgcaggtgccccacgatcacgtggcttacaggtatgaggtcctcaaggtcattgggaaggggagctttgggcaggtggtcaaggcctacgatcacaaagtccaccagcacgtggccctaaagatggtgcggaatgagaagcgcttccaccggcaagcagcggaggagatccgaatcctggaacacctgcggaagcaggacaaggataacacaatgaatgtcatccatatgctggagaatttcaccttccgcaaccacatctgcatgacgtttgagctgctgagcatgaacctctatgagctcatcaagaagaataaattccagggcttcagtctgcctttggttcgcaagtttgcccactcgattctgcagtgcttggatgctttgcacaaaaacagaataattcactgtgaccttaagcccgagaacattttgttaaagcagcagggtagaagcggtattaaagtaattgattttggctccagttgttacgagcatcagcgtgtctacacgtacatccagtcgcgtttttaccgggctccagaagtgatccttggggccaggtatggcatgcccattgatatgtggagcctgggctgcattttagcagagctcctgacgggttaccccctcttgcctggggaagatgaaggggaccagctggcctgtatgattgaactgttgggcatgccctcacagaaactgctggatgcatccaaacgagccaaaaattttgtgagctccaagggttatccccgttactgcactgtcacgactctctcagatggctctgtggtcctaaacggaggccgttcccggagggggaaactgaggggcccaccggagagcagagagtgggggaacgcgctgaaggggtgtgatgatccccttttccttgacttcttaaaacagtgtttagagtgggatcctgcagtgcgcatgaccccaggccaggctttgcggcacccctggctgaggaggcggttgccaaagcctcccaccggggagaaaacgtcagtgaaaaggataactgagagcaccggtgctatcacatctatatccaagttacctccaccttctagctcagcttccaaactgaggactaatttggcgcagatgacagatgccaatgggaatattcagcagaggacagtgttgccaaaacttgttagctga

SEQ ID NO: 37 - protein, 528 aa, Artificial
  Human DYRK2 protein isoform 2
MNDHLHVGSHAHGQIQVQQLFEDNSNKRTVLTTQPNGLTTVGKTGLPVVPERQLDSIHRRQGSSTSLKSMEGMGKVKATPMTPEQAMKQYMQKLTAFEHHEIFSYPEIYFLGLNAKKRQGMTGGPNNGGYDDDQGSYVQVPHDHVAYRYEVLKVIGKGSFGQVVKAYDHKVHQHVALKMVRNEKRFHRQAAEEIRILEHLRKQDKDNTMNVIHMLENFTFRNHICMTFELLSMNLYELIKKNKFQGFSLPLVRKFAHSILQCLDALHKNRIIHCDLKPENILLKQQGRSGIKVIDFGSSCYEHQRVYTYIQSRFYRAPEVILGARYGMPIDMWSLGCILAELLTGYPLLPGEDEGDQLACMIELLGMPSQKLLDASKRAKNFVSSKGYPRYCTVTTLSDGSVVLNGGRSRRGKLRGPPESREWGNALKGCDDPLFLDFLKQCLEWDPAVRMTPGQALRHPWLRRRLPKPPTGEKTSVKRITESTGAITSISKLPPPSSSASKLRTNLAQMTDANGNIQQRTVLPKLVS

SEQ ID NO: 38 - DNA, 1586 bp, Artificial
  Human DYRK2 cDNA isoform 2
atgaatgatcacctgcatgtcggcagccacgctcacggacagatccaggttcaacagttgtttgaggataacagtaacaagcggacagtgctcacgacacaaccaaatgggcttacaacagtgggcaaaacgggcttgccagtggtgccagagcggcagctggacagcattcatagacggcaggggagctccacctctctaaagtccatggaaggcatggggaaggtgaaagccacccccatgacacctgaacaagcaatgaagcaatacatgcaaaaactcacagccttcgaacaccatgagattttcagctaccctgaaatatatttcttgggtctaaatgctaagaagcgccagggcatgacaggtgggcccaacaatggtggctatgatgatgaccagggatcatatgtgcaggtgccccacgatcacgtggcttacaggtatgaggtcctcaaggtcattgggaaggggagctttgggcaggtggtcaaggcctacgatcacaaagtccaccagcacgtggccctaaagatggtgcggaatgagaagcgcttccaccggcaagcagcggaggagatccgaatcctggaacacctgcggaagcaggacaaggataacacaatgaatgtcatccatatgctggagatttcaccttccgcaaccacatctgcatgacgtttgagctgctgagcatgaacctctatgagctcatcaagaagaataaattccagggcttcagtctgcctttggttcgcaagtttgcccactcgattctgcagtgcttggatgctttgcacaaaaacagaataattcactgtgaccttaagcccgagaacattttgttaaagcagcagggtagaagcggtattaaagtaattgattttggctccagttgttacgagcatcagcgtgtctacacgtacatccagtcgcgtttttaccgggctccagaagtgatccttggggccaggtatggcatgcccattgatatgtggagcctgggctgcattttagcagagctcctgacgggttaccccctcttgcctggggaagatgaaggggaccagctggcctgtatgattgaactgttgggcatgccctcacagaaactgctggatgcatccaaacgagccaaaaattttgtgagctccaagggttatccccgttactgcactgtcacgactctctcagatggctctgtggtcctaaacggaggccgttcccggagggggaaactgaggggcccaccggagagcagagagtgggggaacgcgctgaaggggtgtgatgatccccttttccttgacttcttaaaacagtgtttagagtgggatcctgcagtgcgcatgaccccaggccaggctttgcggcacccctggctgaggaggcggttgccaaagcctcccaccggggagaaaacgtcagtgaaaaggataactgagagcaccggtgctatcacatctatatccaagttacctccaccttctagctcagcttccaaactgaggactaatttggcgcagatgacagatgccaatgggaatattcagcagaggacagtgttgccaaaacttgttagctga

SEQ ID NO: 39 - protein, 588 aa, Artificial
  Human DYRK3 protein isoform 1
MGGTARGPGRKDAGPPGAGLPPQQRRLGDGVYDTFMMIDETKCPPCSNVLCNPSEPPPPRRLNMTTEQFTGDHTQHFLDGGEMKVEQLFQEFGNRKSNTIQSDGISDSEKCSPTVSQGKSSDCLNTVKSNSSSKAPKVVPLTPEQALKQYKHHLTAYEKLEIINYPEIYFVGPNAKKRHGVIGGPNNGGYDDADGAYIHVPRDHLAYRYEVLKIIGKGSFGQVARVYDHKLRQYVALKMVRNEKRFHRQAAEEIRILEHLKKQDKTGSMNVIHMLESFTFRNHVCMAFELLSIDLYELIKKNKFQGFSVQLVRKFAQSILQSLDALHKNKIIHCDLKPENILLKHHGRSSTKVIDFGSSCFEYQKLYTYIQSRFYRAPEIILGSRYSTPIDIWSFGCILAELLTGQPLFPGEDEGDQLACMMELLGMPPPKLLEQSKRAKYFINSKGIPRYCSVTTQADGRVVLVGGRSRRGKKRGPPGSKDWGTALKGCDDYLFIEFLKRCLHWDPSARLTPAQALRHPWISKSVPRPLTTIDKVSGKRVVNPASAFQGLGSKLPPVVGIANKLKANLMSETNGSIPLCSVLPKLIS

SEQ ID NO: 40 - DNA, 1767 bp, Artificial
  Human DYRK3 cDNA isoform 1
atgggaggcacagctcgtgggcctgggcggaaggatgcggggccgcctggggccgggctcccgccccagcagcggaggttgggggatggtgtctatgacaccttcatgatgatagatgaaaccaaatgtcccccctgttcaaatgtactctgcaatccttctgaaccacctccacccagaagactaaatatgaccactgagcagtttacaggagatcatactcagcactttttggatggaggtgagatgaaggtagaacagctgtttcaagaatttggcaacagaaaatccaatactattcagtcagatggcatcagtgactctgaaaaatgctctcctactgtttctcagggtaaaagttcagattgcttgaatacagtaaaatccaacagttcatccaaggcacccaaagtggtgcctctgactccagaacaagccctgaagcaatataaacaccacctcactgcctatgagaaactggaaataattaattatccagaaatttactttgtaggtccaaatgccaagaaaagacatggagttattggtggtcccaataatggagggtatgatgatgcagatggggcctatattcatgtacctcgagaccatctagcttatcgatatgaggtgctgaaaattattggcaaggggagttttgggcaggtggccagggtctatgatcacaaacttcgacagtacgtggccctaaaaatggtgcgcaatgagaagcgctttcatcgtcaagcagctgaggagatccggattttggagcatcttaagaaacaggataaaactggtagtatgaacgttatccacatgctggaaagtttcacattccggaaccatgtttgcatggcctttgaattgctgagcatagacctttatgagctgattaaaaaaaataagtttcagggttttagcgtccagttggtacgcaagtttgcccagtccatcttgcaatctttggatgccctccacaaaaataagattattcactgcgatctgaagccagaaaacattctcctgaaacaccacgggcgcagttcaaccaaggtcattgactttgggtccagctgtttcgagtaccagaagctctacacatatatccagtctcggttctacagagctccagaaatcatcttaggaagccgctacagcacaccaattgacatatggagttttggctgcatccttgcagaacttttaacaggacagcctctcttccctggagaggatgaaggagaccagttggcctgcatgatggagcttctagggatgccaccaccaaaacttctggagcaatccaaacgtgccaagtactttattaattccaagggcataccccgctactgctctgtgactacccaggcagatgggagggttgtgcttgtggggggtcgctcacgtaggggtaaaaagcggggtcccccaggcagcaaagactgggggacagcactgaaagggtgtgatgactacttgtttatagagttcttgaaaaggtgtcttcactgggacccctctgcccgcttgaccccagctcaagcattaagacacccttggattagcaagtctgtccccagacctctcaccaccatagacaaggtgtcagggaaacgggtagttaatcctgcaagtgctttccagggattgggttctaagctgcctccagttgttggaatagccaataagcttaaagctaacttaatgtcagaaaccaatggtagtatacccctatgcagtgtattgccaaaactgattagctag

SEQ ID NO: 41 - protein, 568 aa, Artificial
  Human DYRK3 protein isoform 2
MKWKEKLGDGVYDTFMMIDETKCPPCSNVLCNPSEPPPPRRLNMTTEQFTGDHTQHFLDGGEMKVEQLFQEFGNRKSNTIQSDGISDSEKCSPTVSQGKSSDCLNTVKSNSSSKAPKVVPLTPEQALKQYKHHLTAYEKLEIINYPEIYFVGPNAKKRHGVIGGPNNGGYDDADGAYIHVPRDHLAYRYEVLKIIGKGSFGQVARVYDHKLRQYVALKMVRNEKRFHRQAAEEIRILEHLKKQDKTGSMNVIHMLESFTFRNHVCMAFELLSIDLYELIKKNKFQGFSVQLVRKFAQSILQSLDALHKNKIIHCDLKPENILLKHHGRSSTKVIDFGSSCFEYQKLYTYIQSRFYRAPEIILGSRYSTPIDIWSFGCILAELLTGQPLFPGEDEGDQLACMMELLGMPPPKLLEQSKRAKYFINSKGIPRYCSVTTQADGRVVLVGGRSRRGKKRGPPGSKDWGTALKGCDDYLFIEFLKRCLHWDPSARLTPAQALRHPWISKSVPRPLTTIDKVSGKRVVNPASAFQGLGSKLPPVVGIANKLKANLMSETNGSIPLCSVLPKLIS

SEQ ID NO: 42 - DNA, 1707 bp, Artificial
  Human DYRK3 cDNA isoform 2
atgaagtggaaagagaagttgggggatggtgtctatgacaccttcatgatgatagatgaaaccaaatgtcccccctgttcaaatgtactctgcaatccttctgaaccacctccacccagaagactaaatatgaccactgagcagtttacaggagatcatactcagcactttttggatggaggtgagatgaaggtagaacagctgtttcaagaatttggcaacagaaaatccaatactattcagtcagatggcatcagtgactctgaaaaatgctctcctactgtttctcagggtaaaagttcagattgcttgaatacagtaaaatccaacagttcatccaaggcacccaaagtggtgcctctgactccagaacaagccctgaagcaatataaacaccacctcactgcctatgagaaactggaaataattaattatccagaaatttactttgtaggtccaaatgccaagaaaagacatggagttattggtggtcccaataatggagggtatgatgatgcagatggggcctatattcatgtacctcgagaccatctagcttatcgatatgaggtgctgaaaattattggcaaggggagttttgggcaggtggccagggtctatgatcacaaacttcgacagtacgtggccctaaaaatggtgcgcaatgagaagcgctttcatcgtcaagcagctgaggagatccggattttggagcatcttaagaaacaggataaaactggtagtatgaacgttatccacatgctggaaagtttcacattccggaaccatgtttgcatggcctttgaattgctgagcatagacctttatgagctgattaaaaaaaataagtttcagggttttagcgtccagttggtacgcaagtttgcccagtccatcttgcaatctttggatgccctccacaaaaataagattattcactgcgatctgaagccagaaaacattctcctgaaacaccacgggcgcagttcaaccaaggtcattgactttgggtccagctgtttcgagtaccagaagctctacacatatatccagtctcggttctacagagctccagaaatcatcttaggaagccgctacagcacaccaattgacatatggagttttggctgcatccttgcagaacttttaacaggacagcctctcttccctggagaggatgaaggagaccagttggcctgcatgatggagcttctagggatgccaccaccaaaacttctggagcaatccaaacgtgccaagtactttattaattccaagggcataccccgctactgctctgtgactacccaggcagatgggagggttgtgcttgtggggggtcgctcacgtaggggtaaaaagcggggtcccccaggcagcaaagactgggggacagcactgaaagggtgtgatgactacttgtttatagagttcttgaaaaggtgtcttcactgggacccctctgcccgcttgaccccagctcaagcattaagacacccttggattagcaagtctgtccccagacctctcaccaccatagacaaggtgtcagggaaacgggtagttaatcctgcaagtgctttccagggattgggttctaagctgcctccagttgttggaatagccaataagcttaaagctaacttaatgtcagaaaccaatggtagtatacccctatgcagtgtattgccaaaactgattagctag

SEQ ID NO: 43 - protein, 520 aa, Artificial
  Human DYRK4 protein isoform 1
MPASELKASEIPFHPSIKTQDPKAEEKSPKKQKVTLTAAEALKLFKNQLSPYEQSEILGYAELWFLGLEAKKLDTAPEKFSKTSFDDEHGFYLKVLHDHIAYRYEVLETIGKGSFGQVAKCLDHKNNELVALKIIRNKKRFHQQALMELKILEALRKKDKDNTYNVVHMKDFFYFRNHFCITFELLGINLYELMKNNNFQGFSLSIVRRFTLSVLKCLQMLSVEKIIHCDLKPENIVLYQKGQASVKVIDFGSSCYEHQKVYTYIQSRFYRSPEVILGHPYDVAIDMWSLGCITAELYTGYPLFPGENEVEQLACIMEVLGLPPAGFIQTASRRQTFFDSKGFPKNITNNRGKKRYPDSKDLTMVLKTYDTSFLDFLRRCLVWEPSLRMTPDQALKHAWIHQSRNLKPQPRPQTLRKSNSFFPSETRKDKVQGCHHSSRKADEITKETTEKTKDSPTKHVQHSGDQQDCLQHGADTVQLPQLVDAPKKSEAAVGAEVSMTSPGQSKNFSLKNTNVLPPIV

SEQ ID NO: 44 - DNA, 1563 bp, Artificial
  Human DYRK4 cDNA isoform 1
atgccggcctcagagctcaaggcttcagaaatacctttccaccctagcattaaaacccaggatcccaaggcagaggagaagtcaccaaagaagcaaaaggtgactctgacagcggcagaggccctaaagctttttaagaaccagctgtctccatatgaacaaagtgaaatcctgggctacgcggagctgtggttcctgggtcttgaagccaagaagctcgacacggctcctgagaaatttagcaagacgagttttgatgatgagcatggcttctatctgaaggtcctgcatgatcacattgcctaccgctatgaagttctggagacaatcgggaaggggtcctttggacaggtggccaagtgcttggatcacaaaaacaatgagctggtggccctgaaaatcatcaggaacaagaagaggtttcaccagcaggccctgatggagctgaagatcctggaagctctcagaaagaaggacaaagacaacacctacaatgtggtgcatatgaaggactttttctactttcgcaatcacttctgcatcacctttgagctcctgggaatcaacttgtatgagttgatgaagaataacaactttcaaggcttcagtctgtccatagttcggcgcttcactctctctgttttgaagtgcttgcagatgctttcggtagagaaaatcattcactgtgatctcaagcccgaaaatatagtgctataccaaaagggccaagcctctgttaaagtcattgactttggatcaagctgttatgaacaccagaaagtatacacgtacatccaaagccggttctaccgatccccagaagtgatcctgggccacccctacgacgtggccattgacatgtggagcctgggctgcatcacggcggagttgtacacgggctaccccctgttccccggggagaatgaggtggagcagctggcctgcatcatggaggtgctgggtctgccgccagccggcttcattcagacagcctccaggagacagacattctttgattccaaaggttttcctaaaaatataaccaacaacagggggaaaaaaagatacccagattccaaggacctcacgatggtgctgaaaacctatgacaccagcttcctggactttctcagaaggtgtttggtatgggaaccttctcttcgcatgaccccggaccaggccctcaagcatgcttggattcatcagtctcggaacctcaagccacagcccaggccccagaccctgaggaaatccaattcctttttcccctctgagacaaggaaggacaaggttcaaggctgtcatcactcgagcagaaaagcagatgagatcaccaaagagactacagagaaaacaaaagatagccccacgaagcatgttcagcattcaggtgatcagcaggactgtctccagcacggagctgacactgttcagctgcctcaactggtagacgctcccaagaagtcagaggcagctgtcggggcggaggtgtccatgacctccccaggacagagcaaaaacttctccctcaagaacacaaacgttttaccccctattgtatga

SEQ ID NO: 45 - protein, 519 aa, Artificial
  Human DYRK4 protein isoform 2
MPASELKASEIPFHPSIKTQDPKAEEKSPKKQKVTLTAAEALKLFKNQLSPYEQSEILGYAELWFLGLEAKKLDTAPEKFSKTSFDDEHGFYLKVLHDHIAYRYEVLETIGKGSFGQVAKCLDHKNNELVALKIIRNKKRFHQQALMELKILEALRKKDKDNTYNVVHMKDFFYFRNHFCITFELLGINLYELMKNNNFQGFSLSIVRRFTLSVLKCLQMLSVEKIIHCDLKPENIVLYQKGQASVKVIDFGSSCYEHQKVYTYIQSRFYRSPEVILGHPYDVAIDMWSLGCITAELYTGYPLFPGENEVEQLACIMEVLGLPPAGFIQTASRRQTFFDSKGFPKNITNNRGKKRYPDSKDLTMVLKTYDTSFLDFLRRCLVWEPSLRMTPDQALKHAWIHQSRNLKPQPRPQTLRKSNSFFPSETRKDKVQGCHHSSRKDEITKETTEKTKDSPTKHVQHSGDQQDCLQHGADTVQLPQLVDAPKKSEAAVGAEVSMTSPGQSKNFSLKNTNVLPPIV

SEQ ID NO: 46 - DNA, 1560 bp, Artificial
  Human DYRK4 cDNA isoform 2
atgccggcctcagagctcaaggcttcagaaatacctttccaccctagcattaaaacccaggatcccaaggcagaggagaagtcaccaaagaagcaaaaggtgactctgacagcggcagaggccctaaagctttttaagaaccagctgtctccatatgaacaaagtgaaatcctgggctacgcggagctgtggttcctgggtcttgaagccaagaagctcgacacggctcctgagaaatttagcaagacgagttttgatgatgagcatggcttctatctgaaggtcctgcatgatcacattgcctaccgctatgaagttctggagacaatcgggaaggggtcctttggacaggtggccaagtgcttggatcacaaaaacaatgagctggtggccctgaaaatcatcaggaacaagaagaggtttcaccagcaggccctgatggagctgaagatcctggaagctctcagaaagaaggacaaagacaacacctacaatgtggtgcatatgaaggactttttctactttcgcaatcacttctgcatcacctttgagctcctgggaatcaacttgtatgagttgatgaagaataacaactttcaaggcttcagtctgtccatagttcggcgcttcactctctctgttttgaagtgcttgcagatgctttcggtagagaaaatcattcactgtgatctcaagcccgaaaatatagtgctataccaaaagggccaagcctctgttaaagtcattgactttggatcaagctgttatgaacaccagaaagtatacacgtacatccaaagccggttctaccgatccccagaagtgatcctgggccacccctacgacgtggccattgacatgtggagcctgggctgcatcacggcggagttgtacacgggctaccccctgttccccggggagaatgaggtggagcagctggcctgcatcatggaggtgctgggtctgccgccagccggcttcattcagacagcctccaggagacagacattctttgattccaaaggttttcctaaaaatataaccaacaacagggggaaaaaaagatacccagattccaaggacctcacgatggtgctgaaaacctatgacaccagcttcctggactttctcagaaggtgtttggtatgggaaccttctcttcgcatgaccccggaccaggccctcaagcatgcttggattcatcagtctcggaacctcaagccacagcccaggccccagaccctgaggaaatccaattcctttttcccctctgagacaaggaaggacaaggttcaaggctgtcatcactcgagcagaaaagatgagatcaccaaagagactacagagaaaacaaaagatagccccacgaagcatgttcagcattcaggtgatcagcaggactgtctccagcacggagctgacactgttcagctgcctcaactggtagacgctcccaagaagtcagaggcagctgtcggggcggaggtgtccatgacctccccaggacagagcaaaaacttctccctcaagaacacaaacgttttaccccctattgtatga

SEQ ID NO: 47 - protein, 635 aa, Artificial
  Human DYRK4 protein isoform 3
MQLLPPPIRTGTKTQMDAKKPRKCDLTPFLVLKARKKQKFTSAKVGSKLSVQIQKPPSNIKNSRMTQVFHKNTSVTSLPFVDTKGKKNTVSFPHISKKVLLKSSLLYQENQAHNQMPASELKASEIPFHPSIKTQDPKAEEKSPKKQKVTLTAAEALKLFKNQLSPYEQSEILGYAELWFLGLEAKKLDTAPEKFSKTSFDDEHGFYLKVLHDHIAYRYEVLETIGKGSFGQVAKCLDHKNNELVALKIIRNKKRFHQQALMELKILEALRKKDKDNTYNVVHMKDFFYFRNHFCITFELLGINLYELMKNNNFQGFSLSIVRRFTLSVLKCLQMLSVEKIIHCDLKPENIVLYQKGQASVKVIDFGSSCYEHQKVYTYIQSRFYRSPEVILGHPYDVAIDMWSLGCITAELYTGYPLFPGENEVEQLACIMEVLGLPPAGFIQTASRRQTFFDSKGFPKNITNNRGKKRYPDSKDLTMVLKTYDTSFLDFLRRCLVWEPSLRMTPDQALKHAWIHQSRNLKPQPRPQTLRKSNSFFPSETRKDKVQGCHHSSRKADEITKETTEKTKDSPTKHVQHSGDQQDCLQHGADTVQLPQLVDAPKKSEAAVGAEVSMTSPGQSKNFSLKNTNVLPPIV

SEQ ID NO: 48 - protein, 644 aa, Artificial
  Human DYRK4 protein isoform 4
MQLLPPPIRTGTKTQMDAKKPRKCDLTPFLVLKARKKQKFTSAKGPTLSEIYMVGSKLSVQIQKPPSNIKNSRMTQVFHKNTSVTSLPFVDTKGKKNTVSFPHISKKVLLKSSLLYQENQAHNQMPASELKASEIPFHPSIKTQDPKAEEKSPKKQKVTLTAAEALKLFKNQLSPYEQSEILGYAELWFLGLEAKKLDTAPEKFSKTSFDDEHGFYLKVLHDHIAYRYEVLETIGKGSFGQVAKCLDHKNNELVALKIIRNKKRFHQQALMELKILEALRKKDKDNTYNVVHMKDFFYFRNHFCITFELLGINLYELMKNNNFQGFSLSIVRRFTLSVLKCLQMLSVEKIIHCDLKPENIVLYQKGQASVKVIDFGSSCYEHQKVYTYIQSRFYRSPEVILGHPYDVAIDMWSLGCITAELYTGYPLFPGENEVEQLACIMEVLGLPPAGFIQTASRRQTFFDSKGFPKNITNNRGKKRYPDSKDLTMVLKTYDTSFLDFLRRCLVWEPSLRMTPDQALKHAWIHQSRNLKPQPRPQTLRKSNSFFPSETRKDKVQGCHHSSRKADEITKETTEKTKDSPTKHVQHSGDQQDCLQHGADTVQLPQLVDAPKKSEAAVGAEVSMTSPGQSKNFSLKNTNVLPPIV

SEQ ID NO: 49 - protein, 517 aa, Artificial
  Human DYRK4 protein isoform 5
MPASELKASEIPFHPSIKTQDPKAEEKSPKKQKVTLTAAEALKLFKNQLSPYEQSEILGYAELWFLGLEAKKLDTAPEKFSKTSFDDEHGFYLKVLHDHIAYRYEVLETIGKGSFGQVAKCLDHKNNELVALKIIRNKKRFHQQALMELKILEALRKKDKDNTYNVVHMKDFFYFRNHFCITFELLGINLYELMKNNNFQGFSLSIVRRFTLSVLKCLQMLSVEKIIHCDLKPENIVLYQKGQASVKVIDFGSSCYEHQKVYTYIQSRFYRSPEVILGHPYDVAIDMWSLGCITAELYTGYPLFPGENEVEQLACIMEVLGLPPAGFIQTASRRQTFFDSKGFPKNITNNRGKKRYPDSKDLTMVLKTYDTSFLDFLRRWEPSLRMTPDQALKHAWIHQSRNLKPQPRPQTLRKSNSFFPSETRKDKVQGCHHSSRKADEITKETTEKTKDSPTKHVQHSGDQQDCLQHGADTVQLPQLVDAPKKSEAAVGAEVSMTSPGQSKNFSLKNTNVLPPIV